(19) United States
(12) Patent Application Publication
(10) Pub. No.: US 2026/0242818 A1
(43) Pub. Date: Aug. 20, 2026

(54) HYBRID SOYBEAN

(71) Applicants: Donald Danforth Plant Science Center, St Louis, MO (US); THE CURATORS OF THE UNIVERSITY OF MISSOURI, Columbia, MO (US); Cornell University, Ithaca, NY (US)

(72) Inventors: Blake Meyers, Davis, CA (US); MARGARET FRANK, Ithaca, NY (US); Nicole SZELUGA, Ithaca, NY (US)

(21) Appl. No.: 19/161,919

(22) PCT Filed: Apr. 19, 2024
(86) PCT No.: PCT/US2024/025451

§ 371 Date: Sep. 3, 2025

(51) Int. Cl. C12N 15/82; C07K 14/32; C07K 14/415; C12N 9/22; C12N 15/11

(52) U.S. Cl. CPC C12N 15/8289; C07K 14/32; C07K 14/415; C12N 9/226; C12N 15/11; C12N 15/827; C12N 2310/20

(57) ABSTRACT

Genetically modified legume plants comprising a reversibly-sterile phenotype or an improved plant-pollinator phenotype are disclosed. Methods of using the plants to produce hybrid legume seed are also disclosed.

### CROSS-REFERENCE TO RELATED APPLICATIONS

This application claims priority from U.S. Provisional patent application No. 63/497,129, filed Apr. 19, 2023 and U.S. Provisional Patent Application No. 63/614,331 filed on Dec. 22, 2023. The contents of each of the aforementioned applications is hereby incorporated by reference in its entirety.

### INCORPORATION OF SEQUENCE LISTING

The present application contains a Sequence Listing that has been submitted in .XML format via PatentCenter and is hereby incorporated herein by reference in its entirety. Said .XML was created on Apr. 16, 2024, 2024, is named DDPSC0128-401-PCT, and is 443 kilobytes in size.

### FIELD OF THE INVENTION

The present disclosure relates generally to genetically modified legume plants comprising a reversibly sterile phenotype and/or an improved plant-pollinator floral phenotype and methods of using the plants to produce hybrid seed.

### BACKGROUND OF THE INVENTION

Hybrid vigor, also known as heterosis, is the phenomenon in which offspring outperform both of their parents and has been used for over a century to increase crop yields, improve abiotic and biotic stress tolerance, and enhance the nutritional quality of seeds. Unlike crops that have benefited from hybrid vigor, such as maize, soybean has yet to reap the benefits of a large hybrid breeding program. Based on previous small-scale trials, soybean hybrids made through laborious hand crossing techniques can produce a 10-20% increase in yield relative to inbred parents. Due to the challenging nature of producing soybean hybrids, these trials are limited to a small number of genotype combinations, indicating that the potential for yield improvement through heterosis is far from exhausted. To capture the benefits of hybrid breeding, the production of high-yielding and commercially available hybrid soybean seeds must be efficient, affordable, and compatible with existing farming practices.

One approach to increase outcrossing without emasculating flowers is to block self-fertilization using male-sterility (MS). The combination of nuclear male-sterility (NMS), cytoplasmic male-sterility (CMS), and photoperiod/temperature-sensitive genic male-sterility systems with fertility restorer genes have produced promising results in small trials of soybean hybrids. However, multiple drawbacks exist in these systems, making them near impractical for commercial hybrid soybean breeding applications. For example, the three-line breeding system requires a maintainer line, results in low genetic diversity occurring from inbreeding CMS lines and requires restrictive growth conditions in the case of photoperiod/temperature-sensitive male-sterility systems. Most importantly, many of these male-sterile lines are not 100% effective at blocking self-fertilization and often fail to exhibit a full rescue of fertility in the F1 generation, making these existing solutions for hybrid breeding commercially inviable.

Accordingly, there is a need for effective hybrid seed production, and methods for controlled male sterility in legumes such as soybean for effective production of hybrid seed in these economically essential plants.

### SUMMARY OF THE INVENTION

In some aspects, the disclosure encompasses a genetically modified legume plant (Fabaceae) cell, legume plant, part thereof, or legume plant seed thereof comprising a reversible sterility phenotype, the genetically modified plant comprising: (a) a sterility expression construct comprising a promoter operably linked to a sterility polynucleotide encoding a sterility factor; (b) a fertility restorer expression construct comprising a promoter operably linked to a fertility restorer polynucleotide encoding a fertility restorer; or (a) and (b); wherein expression of the sterility factor in the plant causes the plant to be sterile and wherein expression of the fertility restorer in the plant of (b) neutralizes, blocks, offsets, prevents, or inhibits the sterility factor causing the plant to be fertile.

In some aspects, crossing a sterile plant to a fertility restorer plant produces fertile progeny plants. In some aspects, the sterile plant is male-sterile.

In some aspects, the sterility factor is barnase protein and wherein the fertility restorer is barstar protein. In some aspects, the sterility expression construct comprises a tapetum-specific promoter operably linked to a polynucleotide encoding barnase, wherein expression of the barnase in a stamen cell of the plant, kills or disables the stamen cell; wherein the fertility restorer expression construct comprises a tapetum-specific promoter operably linked to a polynucleotide encoding barstar; wherein crossing the sterile genetically modified plant to the fertility restorer genetically modified plant produces progeny comprising the sterility expression construct and the fertility restorer expression construct; and wherein said progeny are fertile due to neutralization, blocking, prevention, or inhibition of barnase activity by barstar. In certain aspects, the tapetum-specific promoter comprises a nucleic acid sequence comprising at least about 75% or more, at least about 85% or more, at least about 95% or more, or 100% sequence identity with a nucleic acid sequence of SEQ ID NO: 38 or SEQ ID NO: 39. In some aspects, the barnase comprises an amino acid sequence comprising at least about 75% or more, at least about 85% or more, at least about 95% or more, or 100% sequence identity with an amino acid sequence of SEQ ID NO: 40. In some aspects, barstar comprises an amino acid sequence comprising at least about 75% or more, at least about 85% or more, at least about 95% or more, or 100% sequence identity with an amino acid sequence of SEQ ID NO: 41.

In further aspects, provided herein are one or more expression constructs for generating a legume plant, plant part, or plant cell thereof, comprising a reversible sterility phenotype, the one or more expression constructs comprising: a sterility expression construct comprising a promoter operably linked to a sterility polynucleotide encoding a sterility factor; and a fertility restorer expression construct comprising a promoter operably linked to a fertility restorer polynucleotide encoding a fertility restorer. In some aspects, the sterility factor is barnase protein and wherein the fertility restorer is barstar.

In certain aspects, the one or more expression constructs disclosed herein, comprises a sterility expression construct comprising a tapetum-specific promoter operably linked to a polynucleotide encoding barnase and wherein the fertility restorer expression construct comprises a tapetum-specific promoter operably linked to a polynucleotide encoding barstar. In some aspects, the sterility expression construct comprises a nucleic acid sequence comprising at least about 75% or more, at least about 85% or more, at least about 95% or more, or 100% sequence identity with a nucleic acid sequence of SEQ ID NO: 42. In some aspects, the fertility restorer expression construct comprises a nucleic acid sequence comprising at least about 75% or more, at least about 85% or more, at least about 95% or more, or 100% sequence identity with a nucleic acid sequence of SEQ ID NO: 43.

The disclosure further encompasses one or more genetically modified plants, plant cells, or parts thereof comprising one or more nucleic acid constructs disclosed herein.

In some aspects, provided herein is a genetically modified legume plant (Fabaceae), part thereof, or plant cell thereof, comprising an improved plant-pollinator floral phenotype, the genetically modified plant comprising a genetic modification of at least one target nucleic acid sequence in a polynucleotide encoding a floral trait, wherein the genetic modification modifies expression of the floral trait, wherein the modified expression of the floral trait improves a plant-pollinator floral phenotype when compared to a plant that does not comprise the genetic modification, wherein the improved plant-pollinator floral phenotype increases rate of cross-pollination of the genetically modified plant with a second plant when compared to the rate of cross-pollination between wild type plants.

In some aspects, the floral trait is flower shape, flower size, flower color, flower odor, nectar composition, nectar volume, or any combination thereof. In some aspects, the floral trait is purple petal color, large petals, sweet nectar, or any combination thereof. In some aspects, the genetic modification modifies expression of a polynucleotide encoding a large petal phenotype.

In some aspects of the genetically modified plant, the polynucleotide encoding a large petal phenotype encodes an AP2-like ethylene-responsive transcription factor (ANT). In some aspects, the genetic modification increases expression of ANT. In some aspects, the genetic modification comprises a polynucleotide encoding a programmable transcription regulation system comprising a programmable targeting domain and a transcription factor domain for transcriptional regulation of the polynucleotide encoding ANT. In some aspects, the programmable transcription regulation system comprises a CRISPR/Cas nuclease system modified for transcriptional regulation of a locus, wherein the CRISPR/Cas nuclease system comprises an engineered protein comprising a nuclease-deficient Cas9 protein (dCAS9 protein) fused to a transcriptional activator, and a guide RNA (gRNA). In some aspects, the engineered protein comprises dCas9 fused at its C terminus to a polypeptide comprising six copies of TALE TAD (Tail-TV CT engineered protein) fused at its C terminus to VP128 (dCas9-TV) and the gRNA comprises a sequence which binds to a polynucleotide sequence in a promoter region of the polynucleotide encoding ANT, to thereby target the engineered protein to the polynucleotide encoding ANT.

In some aspects, the dCas9 protein comprises an amino acid sequence comprising at least about 75% or more, at least about 85% or more, at least about 95% or more, or 100% sequence identity with an amino acid sequence of SEQ ID NO: 24 and the polypeptide comprising six copies of TALE TAD (TAL) fused at its C terminus to VP128 (dCas9-TV) protein comprises at least about 75% or more, at least about 85% or more, at least about 95% or more, or 100% sequence identity with an amino acid sequence of SEQ ID NO: 48.

In some aspects, the genetically modified plant disclosed herein comprises a programmable transcription regulation system which is expressed in flower cells. In some aspects, the polynucleotide encoding the programmable transcription regulation system comprises: an expression construct for expressing dCas9-TV, wherein the expression construct comprises an AP3 promoter (pAP3; SEQ ID NO: 36) comprising a nucleic acid sequence comprising at least about 75% or more, at least about 85% or more, at least about 95% or more, or 100% sequence identity with a nucleic acid sequence of SEQ ID NO: 36, operably linked to a polynucleotide encoding dCas9-TV, wherein the polynucleotide encoding dCas9-TV comprises at least about 75% or more, at least about 85% or more, at least about 95% or more, or 100% sequence identity with a nucleic acid sequence of SEQ ID NO: 66; and one or more expression constructs for expressing the gRNA wherein the one or more expression constructs comprise a pU10 promoter comprising a nucleic acid sequence comprising at least about 75% or more, at least about 85% or more, at least about 95% or more, or 100% sequence identity with a nucleic acid sequence of SEQ ID NO: 30, and a polynucleotide encoding a gRNA which binds to a polynucleotide sequence in a promoter region of the polynucleotide encoding ANT, wherein the polynucleotide encoding the gRNA comprises: a nucleic acid sequence comprising at least about 75% or more, at least about 85% or more, at least about 95% or more, or 100% sequence identity with a nucleic acid sequence of SEQ ID NOs: 83; a nucleic acid sequence comprising at least about 75% or more, at least about 85% or more, at least about 95% or more, or 100% sequence identity with a nucleic acid sequence of SEQ ID NO: 76; or a combination thereof.

In some aspects, the gRNA comprises: a nucleic acid sequence of SEQ ID NO: 1, SEQ ID NO: 2, SEQ ID NO: 3; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 4; a nucleic acid sequence of SEQ ID NO: 5, SEQ ID NO: 6, or SEQ ID NO: 7, or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 8; or a nucleic acid sequence of any combination of SEQ ID NO: 1, SEQ ID NO: 2, SEQ ID NO: 3, SEQ ID NO: 5, SEQ ID NO: 6, and SEQ ID NO: 7, or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 4 or SEQ ID NO: 8.

In some aspects, the polynucleotide encoding the gRNA comprises: an expression construct for expressing gRNA, wherein the expression construct comprises (a) a pU10 promoter comprising a nucleic acid sequence comprising at least about 75% or more, at least about 85% or more, at least about 95% or more, or 100% sequence identity with a nucleic acid sequence of SEQ ID NO: 30, operably linked to a polynucleotide encoding the gRNA comprising SEQ ID NO: 1, SEQ ID NO: 2, SEQ ID NO: 3, wherein the expression construct encoding the gRNA comprises at least about 75% or more, at least about 85% or more, at least about 95% or more, or 100% sequence identity with a nucleic acid sequence of SEQ ID NO: 65; (b) an expression construct for expressing gRNA, wherein the expression construct comprises a pU10 promoter comprising a nucleic acid sequence comprising at least about 75% or more, at least about 85% or more, at least about 95% or more, or 100% sequence identity with a nucleic acid sequence of SEQ ID NO: 30, operably linked to a polynucleotide encoding the gRNA comprising SEQ ID NO: 5, SEQ ID NO: 6, or SEQ ID NO: 7, wherein the expression construct encoding the gRNA comprises at least about 75% or more, at least about 85% or more, at least about 95% or more, or 100% sequence identity with a nucleic acid sequence of SEQ ID NO: 67; or (a) and (b).

In some aspects, the polynucleotide encoding a large petal phenotype encodes a polynucleotide encoding a BIGPETALp (BPEp) protein. In some aspects, the genetic modification reduces expression of BPEp. In some aspects, the genetic modification comprises a polynucleotide encoding a programmable transcription regulation system comprising programmable transcription regulation system comprising a programmable targeting domain and a transcription repressor domain for reducing expression of the polynucleotide encoding BPEp. In some aspects, the programmable transcription regulation system comprises a CRISPR/Cas nuclease system modified for transcriptional regulation of a locus, wherein the CRISPR/Cas nuclease system comprises an engineered protein comprising a nuclease-deficient CAS9 protein (dCAS9) fused to a transcriptional repressor and a guide RNA (gRNA).

In some aspects, the engineered protein comprises a dCAS9 protein fused at its C terminus to a polypeptide comprising an EAR-repression domain (SRDX; dCas9-SRDX) and the gRNA comprises a sequence which binds to a polynucleotide sequence in a promoter region of the polynucleotide encoding BPEp, to thereby target the engineered protein to the polynucleotide encoding BPEp. In some aspects, the dCas9 protein comprises an amino acid sequence comprising at least about 75% or more, at least about 85% or more, at least about 95% or more, or 100% sequence identity with an amino acid sequence of SEQ ID NO: 24 and the polypeptide comprising an EAR-repression domain comprises at least about 75% or more, at least about 85% or more, at least about 95% or more, or 100% sequence identity with an amino acid sequence of SEQ ID NO: 46.

In some aspects, the gRNA comprises a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 17, SEQ ID NO: 18, or both.

In some aspects, the genetically modified plant disclosed herein comprises a programmable transcription regulation system is expressed in flower cells. In some aspects, an expression construct encoding the programmable transcription regulation system comprises: an expression construct for expressing dCas9-SRDX, wherein the expression construct comprises an AP3 promoter (pAP3) comprising a nucleic acid sequence comprising at least about 75% or more, at least about 85% or more, at least about 95% or more, or 100% sequence identity with a nucleic acid sequence of SEQ ID NO: 36, operably linked to a polynucleotide encoding dCas9-SRDX, wherein the polynucleotide encoding dCas9-SRDX comprises at least about 75% or more, at least about 85% or more, at least about 95% or more, or 100% sequence identity with a nucleic acid sequence of SEQ ID NO: 68; and one or more expression constructs for expressing the gRNA wherein the one or more expression constructs comprise a pU10 promoter comprising a nucleic acid sequence comprising at least about 75% or more, at least about 85% or more, at least about 95% or more, or 100% sequence identity with a nucleic acid sequence of SEQ ID NO: 30, and a polynucleotide encoding a gRNA which binds to a polynucleotide sequence in a promoter region of the polynucleotide encoding BPEp, wherein the polynucleotide encoding the gRNA comprises a nucleic acid sequence comprising at least about 75% or more, at least about 85% or more, at least about 95% or more, or 100% sequence identity with a nucleic acid sequence of SEQ ID NO: 74.

In some aspects, the genetically modified plant disclosed herein comprises a genetic modification in a polynucleotide encoding a sweet nectar phenotype. In some aspects, the polynucleotide encoding a sweet nectar phenotype encodes a sucrose-proton symporter 9 (SUC9) sucrose transporter protein. In some aspects, the genetic modification increases expression of SUC9. In some aspects, the genetic modification comprises a polynucleotide encoding a programmable transcription regulation system comprising a programmable transcription regulation system comprising a programmable targeting domain and a transcription factor domain for transcriptional regulation of the polynucleotide encoding SUC9. In some aspects, the programmable transcription regulation system comprises a CRISPR/Cas nuclease system modified for transcriptional regulation of a locus, wherein the CRISPR/Cas nuclease system comprises an engineered protein comprising a nuclease-deficient CAS9 protein (dCAS9) fused to a transcriptional activator and a guide RNA (gRNA).

In some aspects, the engineered protein comprises dCAS9-TV and the gRNA comprises a sequence which binds to a polynucleotide sequence in a promoter region of a polynucleotide encoding SUC9, to thereby target the engineered protein to the polynucleotide encoding SUC9. In some aspects, the programmable transcription regulation system is expressed in flower cells. In some aspects, the gRNA comprises: a nucleic acid sequence of SEQ ID NO: 20, SEQ ID NO: 21, SEQ ID NO: 22, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 26; a nucleic acid sequence of SEQ ID NO: 27, SEQ ID NO: 28, or SEQ ID NO: 29, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 33; or any combination of SEQ ID NO: 20, SEQ ID NO: 21, SEQ ID NO: 22, SEQ ID NO: 27, SEQ ID NO: 28, or SEQ ID NO: 29.

In some aspects, the genetically modified plant of any one of claims 41 to 44, wherein an expression construct encoding the programmable transcription regulation system comprises: an expression construct for expressing dCas9-TV, wherein the expression construct comprises an AGL1 promoter (pAGL1) comprising a nucleic acid sequence comprising at least about 75% or more, at least about 85% or more, at least about 95% or more, or 100% sequence identity with a nucleic acid sequence of SEQ ID NO: 19, operably linked to a polynucleotide encoding dCas9-TV, wherein the polynucleotide encoding dCas9-TV comprises at least about 75% or more, at least about 85% or more, at least about 95% or more, or 100% sequence identity with a nucleic acid sequence of SEQ ID NO: 66; and one or more expression constructs for expressing the gRNA wherein the one or more expression constructs comprise a pU10 promoter comprising a nucleic acid sequence comprising at least about 75% or more, at least about 85% or more, at least about 95% or more, or 100% sequence identity with a nucleic acid sequence of SEQ ID NO: 30, and a polynucleotide encoding a gRNA which binds to a polynucleotide sequence in a promoter region of the polynucleotide encoding SUC9, wherein the polynucleotide encoding the gRNA comprises: (i) a nucleic acid sequence comprising at least about 75% or more, at least about 85% or more, at least about 95% or more, or 100% sequence identity with a nucleic acid sequence of SEQ ID NO: 77, (ii) a nucleic acid sequence comprising at least about 75% or more, at least about 85% or more, at least about 95% or more, or 100% sequence identity with a nucleic acid sequence of SEQ ID NO: 78; or (i) and (ii).

In some aspects, the genetically modified plant disclosed herein comprises a genetic modification which modifies expression of a polynucleotide encoding a purple petal phenotype. In some aspects, the polynucleotide encoding a purple petal phenotype encodes an allele of flower color gene flavonoid 3′5′-hydroxylase (W1) encoding a purple flower (w1) protein. In some aspects, the genetic modification comprises a nucleic acid expression construct comprising a white flower W1 promoter operably linked to a polynucleotide encoding the purple flower w1 protein. In some aspects, the white flower W1 promoter comprises a nucleic acid sequence comprising at least about 75% or more, at least about 85% or more, at least about 95% or more, or 100% sequence identity with a nucleic acid sequence of SEQ ID NO: 34, and wherein the polynucleotide encoding the w1 protein comprises at least about 75% or more, at least about 85% or more, at least about 95% or more, or 100% sequence identity with the nucleic acid sequence of SEQ ID NO: 35.

Further provided herein is an engineered nucleic acid modification system for introducing in a plant a genetic modification in a polynucleotide encoding a plant-pollinator floral phenotype, the system comprising a nucleic acid expression construct comprising: (a) a programmable transcription regulation system targeted to a nucleic acid sequence in a polynucleotide encoding the plant-pollinator floral phenotype; (b) an expression construct comprising a promoter operably linked to a polynucleotide encoding a plant-pollinator floral phenotype operably linked to a promoter; or (a) and (b); wherein the programmable transcription regulation system, the expression construct, or both modify expression of a polynucleotide encoding a plant-pollinator floral phenotype, thereby increasing attraction of a pollinator to a plant, thereby resulting in an increased rate of cross-pollination. In some aspects, the programmable transcription regulation system comprises a programmable targeting domain and a transcription factor domain transcriptional regulation of a locus. In some aspects, the system modified for transcriptional regulation of a locus, wherein the CRISPR/Cas nuclease system comprises an engineered protein comprising a nuclease-deficient CAS9 protein (dCAS9) fused to a transcriptional activator and a guide RNA (gRNA). In some aspects, the engineered protein comprises dCas9 fused at its C terminus to a polypeptide comprising six copies of TALE TAD (TAL) fused at its C terminus to VP128 (dCas9-TV) and the gRNA comprises a sequence which binds to a polynucleotide sequence in a promoter region of a polynucleotide encoding ANT, to thereby target the engineered protein to the polynucleotide encoding ANT.

In some aspects, the engineered nucleic acid modification system disclosed herein comprises: the dCas9-TV protein comprising an amino acid sequence comprising at least about 75% or more, at least about 85% or more, at least about 95% or more, or 100% sequence identity with an amino acid sequence of SEQ ID NO: 9; and the gRNA comprising: a nucleic acid sequence of SEQ ID NO: 1, SEQ ID NO: 2, SEQ ID NO: 3; or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 4; a nucleic acid sequence of SEQ ID NO: 5, SEQ ID NO: 6, SEQ ID NO: 7, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 8; or any combination of SEQ ID NO: 1, SEQ ID NO: 2, SEQ ID NO: 3, SEQ ID NO: 5, SEQ ID NO: 6, and SEQ ID NO: 7.

In some aspects, the engineered protein comprises dCAS9 fused at its C terminus to a polypeptide comprising an EAR-repression domain (SRDX; dCas9-SRDX) and the gRNA comprises a sequence which binds to a polynucleotide sequence in a promoter region of a polynucleotide encoding BPEp, to thereby target the engineered protein to the polynucleotide encoding BPEp. In some aspects, the engineered nucleic acid modification system disclosed herein comprises a dCas9-SRDX protein comprising an amino acid sequence comprising at least about 75% or more, at least about 85% or more, at least about 95% or more, or 100% sequence identity with an amino acid sequence of SEQ ID NO: 10; and a gRNA comprising a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 17, SEQ ID NO: 18, or both.

In some aspects, the engineered protein comprises dCas9 fused at its C terminus to a polypeptide comprising six copies of TALE TAD (TAL) fused at its C terminus to VP128 (dCas9-TV) and the gRNA comprises a sequence which binds to a polynucleotide sequence in a promoter region of the polynucleotide encoding a sucrose-proton symporter 9 (SUC9) sucrose transporter, to thereby target the engineered protein to the polynucleotide encoding the SUC9 sucrose transporter. In some aspects, the dCas9-TV protein comprises an amino acid sequence comprising at least about 75% or more, at least about 85% or more, at least about 95% or more, or 100% sequence identity with an amino acid sequence of SEQ ID NO: 9; and the gRNA comprises: a nucleic acid sequence of SEQ ID NO: 20, SEQ ID NO: 21, SEQ ID NO: 22; or any combination thereof, and wherein the target is a nucleic acid sequence in a polynucleotide in a polynucleotide comprising at least about 75% or more, at least about 85% or more, at least about 95% or more, or 100% sequence identity with a nucleic acid sequence of SEQ ID NO: 26; a nucleic acid sequence of SEQ ID NO: 27, SEQ ID NO: 28, or SEQ ID NO: 29, or any combination thereof and wherein the target is a nucleic acid sequence in a polynucleotide in a polynucleotide comprising at least about 75% or more, at least about 85% or more, at least about 95% or more, or 100% sequence identity with a nucleic acid sequence of SEQ ID NO: 33; or any combination of SEQ ID NO: 20, SEQ ID NO: 21, SEQ ID NO: 22, SEQ ID NO: 27, SEQ ID NO: 28, SEQ ID NO: 29, or any combination thereof.

In some aspects, the genetic modification comprises a nucleic acid expression construct comprising a white flower W1 promoter operably linked to a polynucleotide encoding a purple flower W1 protein. In some aspects, the W1 promoter comprises a nucleic acid sequence comprising at least about 75% or more, at least about 85% or more, at least about 95% or more, or 100% sequence identity with a nucleic acid sequence of SEQ ID NO: 34, and wherein the polynucleotide encoding the W1 protein comprises at least about 75% or more, at least about 85% or more, at least about 95% or more, or 100% sequence identity with the nucleic acid sequence of SEQ ID NO: 35.

Further provided herein are one or more expression constructs for introducing a genetic modification into a legume plant (Fabaceae) of at least one target nucleic acid sequence in a polynucleotide encoding a floral trait, wherein the genetic modification modifies expression of a polynucleotide encoding a plant-pollinator floral phenotype, wherein the modified expression of the floral trait improves a plant-pollinator floral phenotype when compared to a plant that does not comprise the genetic modification, wherein the improved plant-pollinator floral phenotype increases rate of cross-pollination of the genetically modified plant with a second plant when compared to the rate of cross-pollination between wild type plants. In some aspects, the genetic modification comprises a polynucleotide encoding a programmable transcription regulation system comprising a programmable targeting protein modified for transcriptional regulation of a locus. In some aspects, the programmable transcription regulation system comprises a programmable targeting protein modified for transcriptional regulation of a polynucleotide encoding ANT.

In some aspects, the programmable transcription regulation system comprises a CRISPR/Cas nuclease system modified for transcriptional regulation of the polynucleotide encoding ANT, wherein the CRISPR/Cas nuclease system comprises an engineered protein comprising a nuclease-deficient CAS9 protein (dCAS9) fused at its C terminus to a polypeptide comprising six copies of TALE TAD (Tail-TV CT engineered protein) fused at its C terminus to VP128 (dCas9-TV), and a guide RNA (gRNA) comprising a sequence which binds to a polynucleotide sequence in a promoter region of the polynucleotide encoding ANT, to thereby target the engineered protein to the polynucleotide encoding ANT. In some aspects, the expression construct for expressing dCas9-TV (pAP3::dCas9-TV) comprises an AP3 promoter operably linked to a polynucleotide encoding dCas9-TV, wherein the expression construct comprises a nucleic acid sequence comprising at least about 75% or more, at least about 85% or more, at least about 95% or more, or 100% sequence identity with a nucleic acid sequence of SEQ ID NO: 62. In some aspects, the one or more expression constructs for expressing the gRNA comprises an pU10 promoter operably linked to a polynucleotide encoding the gRNA, and wherein the one or more constructs comprise one or more expression constructs for expressing the gRNA and wherein the one or more constructs comprise: (a) a nucleic acid sequence comprising at least about 75% or more, at least about 85% or more, at least about 95% or more, or 100% sequence identity with a nucleic acid sequence of SEQ ID NO: 65; (b) a nucleic acid sequence comprising at least about 75% or more, at least about 85% or more, at least about 95% or more, or 100% sequence identity with a nucleic acid sequence of SEQ ID NO: 67; or (a) and (b).

In some aspects, the programmable transcription regulation system comprises a programmable targeting protein modified for transcriptional regulation of a polynucleotide encoding BPEp. In some aspects, the programmable transcription regulation system comprises a CRISPR/Cas nuclease system modified for transcriptional regulation of the polynucleotide encoding BPEp, wherein the CRISPR/Cas nuclease system comprises an engineered protein comprising a nuclease-deficient CAS9 protein (dCAS9) fused at its C terminus to a polypeptide comprising an EAR-repression domain (SRDX; dCas9-SRDX), and a guide RNA (gRNA) comprising a sequence which binds to a polynucleotide sequence in a promoter region of the polynucleotide encoding BPEp, to thereby target the engineered protein to the polynucleotide encoding BPEp. In some aspects, the expression construct for expressing dCas9-SRDX (pAP3::dCas9-SRDX) comprises an AP3 promoter operably linked to a polynucleotide encoding dCas9-SRDX, wherein the expression construct comprises a nucleic acid sequence comprising at least about 75% or more, at least about 85% or more, at least about 95% or more, or 100% sequence identity with a nucleic acid sequence of SEQ ID NO: 64. In some aspects, the expression construct for expressing the gRNA comprises an pU10 promoter operably linked to a polynucleotide encoding the gRNA, and wherein the expression construct for expressing the gRNA comprises a nucleic acid sequence comprising at least about 75% or more, at least about 85% or more, at least about 95% or more, or 100% sequence identity with a nucleic acid sequence of SEQ ID NO: 70.

In some aspects, the programmable transcription regulation system comprises a programmable targeting protein modified for transcriptional regulation of a polynucleotide encoding SUC9. In some aspects, the programmable transcription regulation system comprises a CRISPR/Cas nuclease system modified for transcriptional regulation of the polynucleotide encoding SUC9, wherein the CRISPR/Cas nuclease system comprises an engineered protein comprising a nuclease-deficient CAS9 protein (dCAS9) fused at its C terminus to a polypeptide comprising six copies of TALE TAD (Tail-TV CT engineered protein) fused at its C terminus to VP128 (dCas9-TV), and a guide RNA (gRNA) comprising a sequence which binds to a polynucleotide sequence in a promoter region of the polynucleotide encoding SUC9, to thereby target the engineered protein to the polynucleotide encoding SUC9. In some aspects, the expression construct for expressing dCas9-TV (pAGL1::dCas9-TV) is under the control of a pAGL1 promoter comprises a nucleic acid sequence comprising at least about 75% or more, at least about 85% or more, at least about 95% or more, or 100% sequence identity with a nucleic acid sequence of SEQ ID NO: 63. In some aspects, the one or more expression constructs for expressing the gRNA comprise a pU10 promoter operably linked to a polynucleotide encoding the gRNA, and wherein the comprise one or more constructs comprise: (a) a nucleic acid sequence comprising at least about 75% or more, at least about 85% or more, at least about 95% or more, or 100% sequence identity with a nucleic acid sequence of SEQ ID NO: 71; (b) a nucleic acid sequence comprising at least about 75% or more, at least about 85% or more, at least about 95% or more, or 100% sequence identity with a nucleic acid sequence of SEQ ID NO: 72; or (a) and (b).

In some aspects, the introduced genetic modification comprises a nucleic acid expression construct comprising a white flower W1 promoter operably linked to a polynucleotide encoding a purple flower W1 protein. In some aspects, the nucleic acid expression construct comprising a white flower W1 promoter operably linked to a polynucleotide encoding the purple flower W1 protein comprises a nucleic acid sequence comprising at least about 75% or more, at least about 85% or more, at least about 95% or more, or 100% sequence identity with a nucleic acid sequence of SEQ ID NO: 73.

The instant disclosure further encompasses one or more genetically modified plant, plant part, or cell thereof comprising one or more nucleic acid constructs disclosed herein.

A genetically modified legume plant (Fabaceae), part thereof, or plant cell thereof, the genetically modified plant comprising a genetic modification in a polynucleotide encoding a floral trait selected from the group consisting of a purple petal phenotype, a large petal phenotype, a sweet nectar phenotype, and any combination thereof; wherein the genetic modification modifies expression of a floral trait, wherein the modified expression of the floral trait improves a plant-pollinator floral phenotype when compared to a plant that does not comprise the genetic modification, wherein the improved plant-pollinator floral phenotype increases rate of cross-pollination of the genetically modified plant with a second plant when compared to the rate of cross-pollination between wild type plants is further disclosed herein.

In some aspects, the disclosure encompasses a genetically modified legume plant (Fabaceae), part thereof, or plant cell thereof, comprising an improved plant-pollinator floral phenotype and a reversible sterility phenotype, the genetically modified plant comprising: an introduced genetic modification in a polynucleotide encoding a plant-pollinator floral phenotype selected from the group consisting of a purple petal phenotype, a large petal phenotype, a sweet nectar phenotype, and any combination thereof and a reversible sterility phenotype, wherein the plant further comprises: (i): a sterility expression construct comprising a promoter operably linked to a sterility polynucleotide encoding a sterility factor; (ii): a fertility restorer expression construct comprising a promoter operably linked to a fertility restorer polynucleotide encoding a fertility restorer; or (iii): (i) and (ii); wherein the genetic modification of (a) increases rate of cross-pollination of the genetically modified plant with a second plant when compared to the rate of cross-pollination between wild type plants; wherein expression of the sterility factor in the plant causes the plant to be sterile and wherein expression of the fertility restorer in the plant of (i) or (iii) neutralizes, blocks, offsets, prevents, or inhibits the sterility factor causing the plant to be fertile; and wherein the combination of the improved plant-pollinator floral phenotype and the reversible sterility phenotype create a synergistic cross pollination phenotype.

Further provided is a method of producing hybrid seed of a legume plant, the method comprising: planting seeds of a sterile genetically modified plant disclosed herein and seeds of a fertility restorer genetically modified plant disclosed herein; allowing the seeds to germinate and grow into plants; and allowing the fertility restorer plants to pollinate the sterile plants to thereby produce the hybrid seed on the sterile plant.

In some aspects, provided herein is a method of producing hybrid seed of a legume plant, the method comprising: planting seeds of genetically modified plants comprising an improved plant-pollinator floral phenotype; and allowing the plants to pollinate to thereby produce the hybrid seed.

In some aspects, disclosed herein is a method of synergistically producing hybrid seed of a legume plant, the method comprising: planting seeds of a sterile genetically modified plant comprising reversible sterility phenotype and an improved plant-pollinator floral phenotype and seeds of a fertility restorer genetically modified plant comprising an improved plant-pollinator floral phenotype; allowing the seeds to germinate and grow into plants; and allowing the fertility restorer plants to pollinate the sterile plants to thereby produce the hybrid seed on the sterile plant; wherein a combination of the improved plant-pollinator floral phenotype and the reversible sterility phenotype create a synergistic cross pollination phenotype.

The disclosure further encompasses a hybrid seed of a legume plant produced using a method disclosed herein.

In some aspects, further provided is a kit for generating a genetically modified legume plant comprising a reversible sterility phenotype, a genetically modified legume plant comprising an improved plant-pollinator phenotype, a genetically modified plant comprising a reversible sterility phenotype and an improved plant-pollinator phenotype, or for producing hybrid seed of the legume plant, the kit comprising: (a) one or more genetically modified plant disclosed herein; (b) one or more expression constructs disclosed herein: (c) one or more genetically modified plants, plant cells, or parts thereof disclosed herein; (d) hybrid seed of a legume plant disclosed herein; or any combination of (a) to (d).

### BRIEF DESCRIPTION OF THE FIGURES

The following drawings form part of the present specification and are included to further demonstrate certain embodiments of the present disclosure. Certain embodiments can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1A is a graphical depiction of a construct designed for tapetum-specific expression of barnase to generate stable and spatially accurate gene expression in soybean anthers. Tapetum-specific expression of barnase driven by the weaker promoter, TA29b. aadA1 is a resistance gene for kanamycin selection.

FIG. 1B is a graphical depiction of a construct designed for tapetum-specific expression of barstar to generate stable and spatially accurate gene expression in soybean anthers. Tapetum-specific expression of barstar driven by the weaker promoter, TA29b. aadA1 is a resistance gene for kanamycin selection.

FIG. 1C. is a graphical depiction of a construct designed for tapetum-specific expression of barstar under stronger promoter, TA29a. This construct design also includes constitutive expression of fluorophore, td_Tomato.

FIG. 1D. Construct design for TA29a/b control line.

FIG. 1E. Anther images of TA29a/b control line were cleared and imaged with confocal microscopy. Red and Green Channels were altered in the Fiji software system to project magenta and yellow, respectively. White scale bar is 100 μm.

FIG. 2 depicts photographs of soybean plants expressing narnase and barstar. no physiological stressors are acting upon TA29b::barnase and TA29a::barstar transformation events by providing top-view images of Wildtype, TA29b::barnase, and TA29a::barstar lines captured with NPEC's CropReporter™. Panels (a) to (c) are color spectrum images pairing with Fv/Fm images in panels (d) to (f) to demonstrate no physiological stress is present in the plant by the appearance of bright green on the Fv/Fm scale. Plants were grown under greenhouse conditions at the Danforth Plant Science Center. Orange scale bar is 100 mm.

FIG. 3 depicts paralogs of TA29 being differentially expressed in flower buds. RNAseq data published on JGI Plant Gene Atlas compares fragments per kilobase of exon per million mapped fragments (FPKM) for TA29a and TA29b.

FIG. 4 depicts images of genetically modified soybean flowers. TA29b::barnase transformation events result in cleistogamous flowers, sterile anthers, and curved carpels. Panels (a) to (i)I? are flower dissection images of four additional positive transformation events of TA29b::barnase. The scale bars are as follows: yellow bar is 1 mm and pink bar is 500 μm.

FIG. 5 depicts images of flower dissection of TA29b::barnase displays cleistogamous flowers, male-sterile anthers, and curved carpels compared to wildtype, TA29a::barstar, and TA29b::barstar. Panels (a), (g), (m), and(s) show flowers dissected and imaged under a dissecting microscope. Panels (b), (h), (n), and (t) show stamen dissected and imaged under a dissecting microscope. Panels (d), (j), (p), and (v) show carpel images dissected and imaged under a dissecting microscope. Whole flower images were captured at peak maturity. Panels (c), (i), (o), and (p) show anther images stained with PI and imagined using confocal microscopy. Panels (e), (k), (g), and (w) show carpel images obtained by staining with PI and imaged using confocal microscopy. Panels (f), (l), (r), and (x) show soybean plants grown under growth chamber conditions and images 90 days post germination. Scale bars for the figure are as follows: blue bar is 1 mm; pink bar is 500 μm; white bar is 100 μm; orange bar is 100 mm.

FIG. 6 depicts male-sterile barnase lines produce parthenocarpic pods. At maturity, wildtype pods elongate (shown in Panels. (a) and (c) and develop seeds (shown in Panel (e). Parthenocarpic pods (shown in Panels (b) and (d) remain short and the ovules never develop into embryos (shown in Panel (f). Panels (b) and (d)-(f) were taken images on a dissecting microscope. The scale bars are as follows: green bar is 10 mm and yellow bar is 1 mm.

FIG. 7 depicts TA29b::barnase male-sterile carpels being receptive to pollination and restore fertility to F1 generation when crossed with TA29a::barstar. Panels (a)-(c) are Aniline blue images capture pollen tube growth through the stile of receptive carpels. Panels (d) and (e) are plants grown in growth chamber conditions and imaged 90 days post germination. Panels (f) and (g) are flower buds imaged at peak reproductive maturity. Panels (h) and (i) are PI-stained anthers dissected from flowers and imaged with a confocal microscope. Scale bars for the following figure are as follows: white bar is 100 μm; orange bar is 100 mm; blue bar is 1 mm.

FIG. 8 depict TA29b::barstar being unable to rescue fertility in the F1 generation. Panels (a) and (b) show additional PI-stained anther images captured by confocal microscopy of failed rescues display consistent male-sterility regardless of TA29b::barnase transformation line. Panels (c) and (d) show PI-stained images of successful rescues highlight the existence of pollen granules in varying barnase/barstar transformation line pairings. Panel (e) shows PI-stained anther of partial rescue develops pollen which clumps together and away from the anther wall. White scale bar is 100 μm.

FIG. 9 depicts partial rescue producing significantly fewer seeds compared to wildtype. Boxplot graphs the significant increase in number of seeds per plant for wildtype compared to partial rescues grown in greenhouse growing conditions (n=22). Statistics were done with Student's t-test.

FIG. 10A is a boxplot comparing qRT-PCR data of barnase and barstar genes from the successful rescue line. barnase and barstar expression are normalized relative to the control, UKN1. Bars extending from the boxplot indicate maximum and minimum and the middle line in the box marks the median data point.

FIG. 10B is a boxplot comparing qRT-PCR data of barnase and barstar genes from the failed rescue line.

FIG. 10C is a boxplot comparing the percent of pollen alive per image taken during a pollen viability assay. The plot compared wildtype to the successful rescue (n=36).

FIG. 10D is a boxplot of total seeds collected per plant taken from wildtype and the failed rescue. Statistics were calculated with the student's t-test.

FIG. 11A depicts images showing that pollen viability assay stains living cells with FDA, emitting green fluorescence at 488 nm emission, and stains dead cells with PI, emitting red fluorescence at 561 nm emission by laser lines on the confocal microscope. In this figure, red color has been converted to magenta. Panels (a)-(c) show wildtype assay images having a significantly higher number of pollen granules per 9-tile frame than successful rescue assay images shown in Panels (d)-(f).

FIG. 11B is a boxplot quantifying pollen viability shown in FIG. 11A (n=37). Statistics were done using Student's t-test. Light pink scale bar is 200 μm.

FIG. 12 depicts diagrammatic representation of the effects of TA29b::barnase, TA29b::barnase×TA29b::barstar (failed rescue), and TA29b::barnase×TA29a::barstar (successful rescue), respectively. Top row Panels (a)-(c) depict barnase, barstar, and mRNA interaction inside the nucleus of a single cell in the tapetum. The tapetal cells are enlarged from a digital drawing of soybean anthers containing four pollen sacs. The diagram outlines the development of the anthers under the influence of barnase and barstar expression. Panel (c) shows the successful rescue mimics wildtype anther growth as it progresses from early anther development, microsporogenesis, and anther maturity. Panels (a)-(b) show pollen development halted prematurely when barnase enzymes are uninhibited and the tapetum is destroyed.

FIG. 13A. Soybean flowers are small and fail to benefit from hybrid breeding. This is partly because soybean produces small, inconspicuous flowers that fail to benefit from hybrid breeding.

FIG. 13B. Soybean flowers are small and fail to benefit from hybrid breeding. This is partly because soybean produces small, inconspicuous flowers that fail to benefit from hybrid breeding.

FIG. 14A. Gene selection and implementation of transcriptional activation/repression to modify floral phenotypes. Target genes for transcriptional activation/repression (highlighted in red) were selected based on their expression in developing floral buds and sequence homology with Arabidopsis genes.

FIG. 14B. Gene selection and implementation of transcriptional activation/repression to modify floral phenotypes. Transcriptional activation/repression and W1 complementation transgenic lines targeting candidates from FIG. 14A will be tested for increased pollinator attraction.

FIG. 14C. Gene selection and implementation of transcriptional activation/repression to modify floral phenotypes. Schematic showing how guide RNAs direct deactivated Cas9 “dCas9” carrying either an activator (VP64) or a repressor (SRDX) to the promoters of target genes to modify gene expression.

FIG. 15A. Field study showing that bee pollinators visit soybean flowers, albeit infrequently. Panel (A) Picture from a field site. Male sterile mutant genotypes (L75, NE1900, and T274H) were interspersed with purple- and white-flowered fertile genotypes. Uncovered male sterile lines could be open-pollinated, while covered male sterile lines were protected from bees and thus all “covered” seed set is assumed to be the product of self-fertilization. Panel (B) Image of a honeybee visiting one of the soybean plants. Infrequent pollinator visitation during the field season was observed.

FIG. 15B. Pilot field study showing that bee pollinators visit soybean flowers, albeit infrequently. Increased seed yield on the open-pollinated male sterile lines relative to the covered lines indicates pollen delivery from the surrounding fertile lines.

FIG. 15C. Pilot field study showing that bee pollinators visit soybean flowers, albeit infrequently. Genotype data for offspring collected from male-sterile lines provides evidence for outcrossing with surrounding male-fertile lines.

FIG. 15D. Pilot field study showing that bee pollinators visit soybean flowers, albeit infrequently. Genotyping for 100 offspring from male sterile mothers, shows that most offspring still form from self-fertilization (64%) because male sterility is not 100% effective in these mutants, with an additional 34% of offspring coming from a purple-flowered pollen donor and 2% from a white-flowered pollen donor.

FIG. 16 Images showing the purple color flowers in plants expressing the purple flower trait.

### DETAILED DESCRIPTION

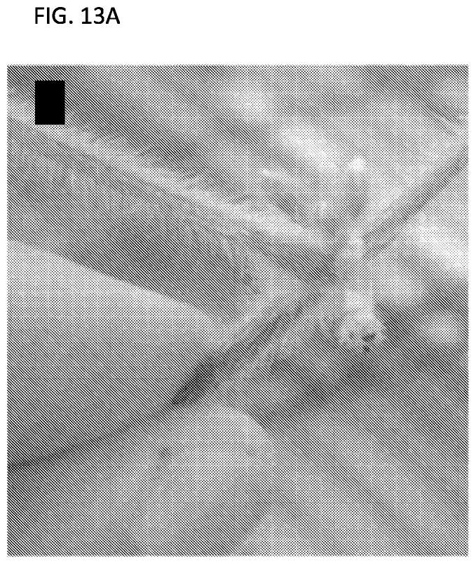

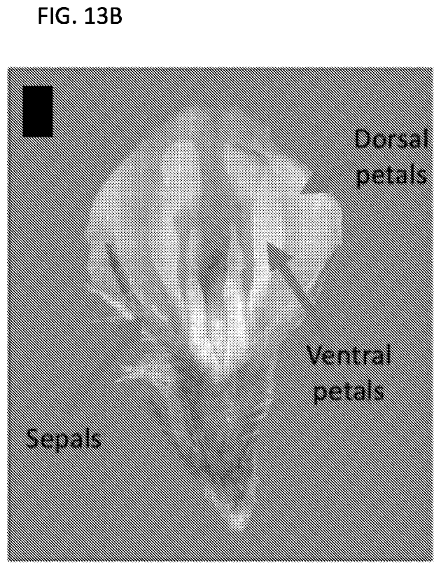

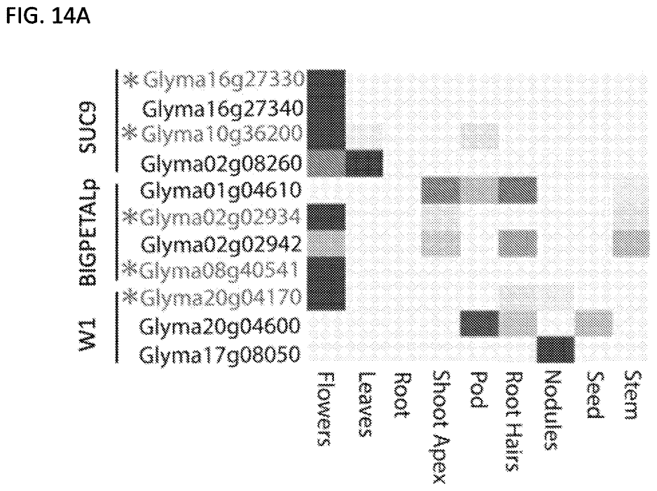

The present disclosure is based in part on the surprising demonstration of reversible male-sterility in legume (Fabaceae) plants where no viable methods of producing hybrid seed exist including in self-pollinating legume plants. The disclosure is also based in part on the discovery by the inventors that changing flower phenotypes (morphology and/or color) in legume plants recruits pollinators to the flower, thereby improving chances of cross pollination. This is especially important in self-fertilizing legume plants that have inconspicuous flowers such as soybeans. Soybean makes inconspicuous flowers that self-fertilize before opening, and thus are not readily amenable to outcrossing using manual methods or (FIG. 13A). When combined in soybeans, the reversible sterility phenotype and the morphology/color phenotype synergistically improve cross pollination and consequently make production of hybrid seed possible and even commercially inviable.

### I. Reversible Sterility

One aspect of the instant disclosure encompasses a genetically modified legume plant (Fabaceae) cell, legume plant, part thereof, or legume seed cell thereof comprising a reversible-sterility phenotype for improving hybrid seed production. As used herein, the term “reversible sterility” is used to refer to a plant comprising reproductive organs that can temporarily lose the ability to produce viable gametes (pollen or eggs) or fail to achieve fertilization, resulting in reduced or no seed production, wherein sterility of the plant can be reversed, or fertility can be restored, by expressing a fertility restorer in the sterile plant. The plants thus cease to self-fertilize for a generation and promote hybrid breeding in legumes, even in self-fertilizing legumes such as soybeans.

The inventors demonstrated that a reversible sterility phenotype of the instant disclosure can be obtained in legumes using a sterility/fertility restorer system, wherein expression of a sterility factor in the plant causes the plant to be reversibly male sterile and wherein expression of a fertility restorer in the sterile plant neutralizes, blocks, offsets, prevents, or inhibits the sterility factor causing the plant to be fertile. Fertility can be restored in the F1 generation of sterile lines when they are crossed with pollen from fertility restorer plants that express a fertility restorer. Accordingly, the genetically modified plant of the instant disclosure can be a sterile plant comprising a polynucleotide encoding a sterility factor; a plant comprising a polynucleotide encoding a fertility restorer; or a plant comprising a polynucleotide encoding a sterility factor and a polynucleotide encoding a fertility restorer.

Legume plants can be as described in Section VI (a). In some aspects, a legume plant of the instant disclosure is a self-fertilizing legume plant. In some aspects, a legume plant of the instant disclosure is soybean.

In some aspects, a sterile genetically modified plant of the instant disclosure comprises a polynucleotide encoding a sterility factor. As used herein, the term “sterility factor” refers to any DNA, RNA, protein, or peptide that, when expressed in the plant disrupts development and/or function of flowers, male or female gametes, embryo, or seeds, causing the flower, male or female gametes, embryo, or seeds to die or be non-functional.

Any sterility factor can be used in a genetically modified plant of the instant disclosure provided the sterility factor can be neutralized, blocked, offset, overcome, prevented, or inhibited, by a fertility restorer that, when expressed in the plant expressing the sterility factor can neutralize, block, offset, prevent, or inhibit the sterility factor to thereby cause the plant to be fertile. Sterility factors are known to individual of skill in the art and include, without limitation, RNAses, proteases, DNAses, or any other molecule that can disrupt the function of a molecule or pathway essential for development of viable gametes (pollen or eggs) or fail to achieve fertilization in response to triggering conditions. Non-limiting examples of sterility factors that can be expressed in a genetically modified plant of the instant disclosure include barnase which degrades RNA molecules by hydrolyzing the bond after any guanine residue; EcoRI endonuclease which digests DNA; or papain such as papain zymogen and papain active protein); enzymes which catalyze the synthesis of phytohormones, such as isopentenyl transferase which is an enzyme that catalyzes the first step in cytokinin biosynthesis glucanases; lipases such as phospholipase A2; lipid peroxidases; plant cell wall inhibitors; and proteins toxic to plants cells, such as a bacterial toxin (e.g., the A-fragment of diphtheria toxin or botulin); as well as the antisense DNAs of the sterility DNAs encoding natural self-incompatibility genes.

In other aspects, the genetically modified plant comprises a polynucleotide encoding a fertility restorer. As used herein, the term “fertility restorer” refers to any DNA, RNA, protein, or peptide that, when expressed in a plant that expresses a sterility factor can neutralize, block, offset, overcome, prevent, or inhibit the activity of the sterility factor to thereby prevent the sterility factor from causing the plant to be sterile and thereby restoring fertility of the plant.

In some aspects, the fertility restorer corresponds and specifically blocks the sterility factor as a sterility/fertility restorer system. Accordingly, if a sterility factor is an RNAse, the fertility restorer can be an RNAse inhibitor. Similarly, if a sterility factor is a protease, the fertility restorer can be a protease inhibitor.

Non-limiting examples of sterility/fertility restorer systems comprising fertility restorers corresponding to sterility factors include the barnase/barstar system, wherein barstar neutralizes the activity of barnase; EcoRI methylase/EcoRI endonuclease system wherein the EcoRI methylase prevents the activity of the EcoRI endonuclease; and a papain/glycosylation factor wherein the glycosylation factor glycosylates papain to inactivate the protease.

Other fertility restorers can include any molecule that can disrupt or neutralize expression of a sterility factor, including nucleic acid modification systems. For instance, the restorer can be an RNAi that targets barnase; EcoRI; glucanases; lipases; plant cell wall inhibitors; and proteins toxic to plant cells. Nucleic acid modification systems can be as described in Section IV herein below.

The genetically modified reversibly sterile plants can be male-sterile or female sterile, wherein male-sterile plants can comprise a sterility expression construct comprising a male organ- or tissue-specific promoter operably linked to a sterility factor and female-sterile plants can comprise a sterility expression construct comprising a female organ- or tissue-specific promoter operably linked to a sterility factor. In some aspects, the fertility restorer construct expresses the fertility restorer in at least those specific female or male organ- or tissue-specific cells of a sterile plant in which the sterility expression construct is expressed. In some aspects, the sterility construct and the fertility restorer construct comprise the same promoter. Promoters including promoters that direct expression on nucleic acid sequences in male or female reproductive organs or tissues can be as described in Section V herein below.

In some aspects, sterile plants of the instant disclosure are male-sterile. In some aspects, male-sterile plants comprise an expression construct for expressing a sterility factor in a male organ or tissue. In some aspects, expressing a sterility factor in a male organ or tissue comprises a male-organ- or tissue-specific promoter operably linked to a polynucleotide encoding a sterility factor. In some aspects, the sterility expression construct comprises a tapetum-specific promoter operably linked to a polynucleotide encoding a sterility factor, wherein expression of the sterility factor in the stamen cell of the plant, kills or disables the stamen cell to thereby produce a male-sterile plant. In wildtype post-meiotic anthers, each pollen sac is composed of a ring of endothecium cells, a middle layer, and a tapetal layer that surrounds developing pollen grains. The role of the tapetum is to provide nutrients and precursor material to build the outer pollen coat for developing grains. Targeted expression of a sterility factor in the tapetum destroys this cell layer, causing male pollen infertility. Fertility is restored in the second generation by crossing sterile plants with pollen carrying a transgene for the tapetum-specific expression of a corresponding fertility restorer.

In some aspects, the male organ- or tissue-specific promoter is a pTA29a version of the pTA29 promoter. Importantly, the inventors unexpectedly discovered that the pTA29b version of the pTA29 promoter was not able to restore fertility of sterile plants expressing barstar under the control of the pTA29b promoter, whereas expressing barstar under the control of a pTA29a promoter was successful restore fertility of sterile plants expressing barstar under the control of the pTA29b promoter.

In some aspects, the pTA29a promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 38. In some aspects, the pTA29a promoter comprises a nucleic acid sequence comprising at least about 75% or more, at least about 85% or more, at least about 95% or more, or 100% sequence identity with a nucleic acid sequence of SEQ ID NO: 38.

In some aspects, the pTA29b promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 39. In some aspects, the pTA29b promoter comprises a nucleic acid sequence comprising at least about 75% or more, at least about 85% or more, at least about 95% or more, or 100% sequence identity with a nucleic acid sequence of SEQ ID NO: 39.

In some aspects, a genetically modified plant of the instant disclosure comprises a genetically modified plant expressing a sterility factor. Accordingly, in some aspects, a genetically modified plant of the instant disclosure comprises a sterility expression construct comprising a promoter operably linked to a sterility polynucleotide encoding a sterility factor.

In some aspects, the genetically modified plant is female-sterile. In some aspects, a genetically modified plant of the instant disclosure comprises a sterility expression construct for expressing the sterility factor in a female organ, wherein the expression construct comprises a promoter operably linked to a sterility polynucleotide encoding a sterility factor. In some aspects, the sterility expression construct comprises a female organ- or tissue-specific promoter operably linked to a polynucleotide encoding a sterility factor, wherein expression of the sterility factor in the female organ or tissue of the plant, kills or disables the female organ to thereby produce a female-sterile plant.

In some aspects, the genetically modified plant is male-sterile. In some aspects, a genetically modified plant of the instant disclosure comprises a sterility expression construct for expressing the sterility factor in a male organ or tissue, wherein the expression construct comprises a promoter operably linked to a sterility polynucleotide encoding a sterility factor. In some aspects, the sterility expression construct comprises a male organ- or tissue-specific promoter operably linked to a polynucleotide encoding a sterility factor, wherein expression of the sterility factor in the male organ or tissue of the plant, kills or disables the female organ to thereby produce a female-sterile plant. In some aspects, the sterility expression construct comprises a tapetum-specific promoter operably linked to a polynucleotide encoding a sterility factor, wherein expression of the sterility factor in the tapetum cell of the plant, kills or disables the stamen cell to thereby produce a female-sterile plant.

In some aspects, a genetically modified plant of the instant disclosure comprises a genetically modified plant expressing a fertility restorer. Accordingly, in some aspects, a genetically modified plant of the instant disclosure comprises a fertility restorer expression construct comprising a promoter operably linked to a fertility restorer polynucleotide encoding a fertility restorer. In some aspects, fertility of the reversibly sterile plants can be restored by crossing the sterile plant with the plant expressing a fertility restorer to produce fertile progeny plants. In some aspects, the fertility restorer is expressed in the same cells as the sterility factors of the corresponding plant. In some aspects, the fertility restorer expression construct comprises a male organ- or tissue-specific promoter operably linked to a polynucleotide encoding a fertility restorer, wherein expression of the fertility restorer in the male organ or tissue of the plant, kills or disables the female organ to thereby produce a female-sterile plant. In some aspects, the fertility restorer expression construct comprises a tapetum-specific promoter operably linked to a polynucleotide encoding a fertility restorer, wherein expression of the fertility restorer in the tapetum cell of the plant, kills or disables the stamen cell to thereby produce a female-sterile plant.

In some aspects, the sterility factor is barnase and the fertility restorer is barstar. The barnase/barstar system is a genetic system that involves the use of two genes, barnase and barstar, originally derived from the bacterium Bacillus amyloliquefaciens. These genes encode for a ribonuclease enzyme (barnase) and its specific inhibitor (barstar). When these genes are introduced into plants, they can be used to create male- or female-sterile plants by disrupting gamete development.

The present disclosure demonstrates that the barnase/barstar sterility/fertility restorer system can be used in legumes, including soybean, to produce hybrid seed. More specifically, the inventors demonstrated that expressing the cytotoxic ribonuclease, barnase, under a tapetum-specific promoter in soybean anthers, it was able to completely block pollen maturation, creating male-sterile plants. It was shown that fertility can be rescued in the F1 generation of these barnase-expressing lines when they are crossed with pollen from plants that express the barnase inhibitor, barstar. Importantly, it was found that successful rescue of male-fertility is dependent on the relative dosage of barnase and barstar. When barnase and barstar were expressed under the same tapetum-specific promoter, the F1 offspring remained male-sterile. When barstar was expressed under a relatively stronger promoter than the promoter used for expressing barnase, it was able to achieve successful rescue of male-fertility in the F1 generation.

Accordingly, in some aspects, the instant disclosure encompasses a genetically modified plant comprising an expression construct for expressing barnase, a genetically modified plant comprising an expression construct for expressing barstar, or a genetically modified plant comprising the expression construct for expressing barnase and the expression construct for expressing barstar.

In some aspects, the sterility expression construct comprises a tapetum-specific promoter operably linked to a polynucleotide encoding barnase, wherein expression of the barnase in a stamen cell of the plant, kills or disables the stamen cell; wherein the fertility restorer expression construct comprises a tapetum-specific promoter operably linked to a polynucleotide encoding barstar; wherein crossing the sterile genetically modified plant to the fertility restorer genetically modified plant produces progeny comprising the sterility expression construct and the fertility restorer expression construct; and wherein said progeny are fertile due to neutralization, blocking, prevention, or inhibition of barnase activity by barstar.

In some aspects, the barnase protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 40. In some aspects, the barnase protein comprises an amino acid sequence comprising at least about 75% or more, at least about 85% or more, at least about 95% or more, or 100% sequence identity with an amino acid sequence of SEQ ID NO: 40.

In some aspects, barstar comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 41. In some aspects, barstar comprises an amino acid sequence comprising at least about 75% or more, at least about 85% or more, at least about 95% or more, or 100% sequence identity with an amino acid sequence of SEQ ID NO: 41.

The sterility expression construct comprises a tapetum-specific promoter operably linked to a polynucleotide encoding barnase which, when directed by the tapetum-specific promoter, is produced or overproduced in a stamen cell of the plant, kills or disables the stamen cell; wherein the fertility restorer expression construct comprises a tapetum-specific promoter operably linked to a polynucleotide encoding barstar; wherein crossing the sterile genetically modified plant to the fertility restorer genetically modified plant produces progeny comprising the sterility expression construct and the fertility restorer expression construct; wherein said progeny are fertile due to neutralization, blocking, prevention, or inhibition of the activity of barnase by barstar.

In some aspects, the sterility expression construct comprises a pTA29b promoter operably linked to a polynucleotide encoding barnase. The sterility expression construct can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 42. The sterility expression construct can comprise a nucleic acid sequence comprising at least about 75% or more, at least about 85% or more, at least about 95% or more, or 100% sequence identity with a nucleic acid sequence of SEQ ID NO: 42.

In some aspects, the fertility restorer expression construct comprises a pTA29a promoter operably linked to a polynucleotide encoding barstar. The fertility restorer expression construct can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 43. The fertility restorer expression construct can comprise a nucleic acid sequence comprising at least about 75% or more, at least about 85% or more, at least about 95% or more, or 100% sequence identity with a nucleic acid sequence of SEQ ID NO: 43.

As explained herein above, the inventors unexpectedly discovered that the pTA29b version of the pTA29 promoter cannot be used in a fertility restorer system of the instant disclosure. In some aspects, the expression construct comprising the pTA29b promoter which was found to be insufficient for restoring fertility comprises a nucleic acid sequencing comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 44. In some aspects, the expression construct comprising the pTA29b promoter comprises a nucleic acid sequencing comprising at least about 75% or more, at least about 85% or more, at least about 95% or more, or 100% sequence identity with a nucleic acid sequence of SEQ ID NO: 44.

### II. Improved Plant-Pollinator Phenotype

Another aspect of the instant disclosure encompasses a genetically modified legume plant (Fabaceae) cell, legume plant, part thereof, or legume plant seed thereof comprising an improved plant-pollinator phenotype. The genetically modified plant comprises a genetic modification of at least one target site in a nucleic acid sequence in a polynucleotide encoding a floral trait. The genetic modification modifies the expression of the flower factor, thereby improving the plant-pollinator floral phenotype when compared to a plant that does not comprise the genetic modification. The improved plant-pollinator floral phenotype improves attraction of pollinators to the flowers, thereby increasing the rate of cross-pollination of the genetically modified plant when compared to the rate of cross-pollination in plants that do not comprise the genetic modification. The rate of cross-pollination is increased by an increase in pollinator visits to the genetically modified plant, attracted by the improved plant-pollinator floral phenotype. Legume plants and pollinators can be as described in Section VI (a) herein below. In some aspects, a legume plant of the instant disclosure is a self-fertilizing legume plant. In some aspects, a legume plant of the instant disclosure is soybean. In some aspects, a pollinator is bumblebees.

Plants use a combination of floral traits or phenotypes to attract pollinators. Non-limiting examples of floral traits that can contribute to attraction of a pollinator to a flower include flower shape, flower size, flower color, flower odor, nectar composition, nectar volume, pollen rewards, timing and duration of flowering, or any combination thereof. These factors can vary depending on the species of the flower and the pollinator intended to be attracted, as well as the environmental conditions in which they interact.

The very complexity of pollinator preference, flower morphology, and associated endless possible combinations of phenotypes makes it impossible to predict which floral trait or combinations of floral traits can be manipulated to improve attraction of pollinators. This is especially true when the plant is self-fertilizing, thus providing no guidance for flower morphology attractive to pollinators. In fact, it is not possible to predict whether any change in a floral trait normally associated with improved pollinator attraction to a flower could also improve pollinator attraction to flowers of a self-pollinating plant. Additionally, although a number of genes that determine flower phenotypes have been identified in many plant species, mainly Arabidopsis sp., the genes and mechanisms that determine flower morphology have yet to be identified in legumes. Accordingly, there is a risk that the genetic mechanisms underlying flower morphology have sufficiently diverged between Arabidopsis and legumes, which necessitates extensive experimentation for identifying and confirming the functions of legume genes of flower morphology.

According to the instant disclosure, the inventors identified floral traits and combinations of floral traits that improve the rate of cross-pollination of legume plants by attracting pollinators. The inventors also identified and generated plants comprising the genetic modifications in nucleic acids encoding the floral traits and demonstrated that the identified floral phenotypes can be used to improve pollinator attraction to flowers of legume plants, including self-pollinating legume plants such as soybean, and thus improve rates of cross-pollination. For this purpose, the inventors first identified previously unknown genes controlling legume floral traits, then modified the genes that control expression of these genes and demonstrated that the modified floral traits increase pollinator attraction to flowers that contain the modified floral traits and enhance cross-pollination of plants. Importantly, the identified floral phenotypes and the genetic modifications identified by the inventors are also capable of improving the rate of cross pollination of self-fertilizing plants. In some aspects, the floral trait is purple petal color, large petals, sweet nectar, or any combination thereof.

### (a) Petal Size and Shape

In some aspects, genetically modified plants of the instant disclosure comprise a genetic modification in a nucleic acid sequence encoding a petal phenotype. Accordingly, in some aspects, a genetically modified plant of the instant disclosure comprises a genetic modification that modifies expression of a polynucleotide encoding a petal phenotype. The size and shape of flower petals can influence visual attraction of pollinators. The size and shape of flower petals can also play a role in flower-pollinator interactions. For instance, flowers with shallow, flat petals are often easier for pollinators with short proboscises, such as bees, to access, while flowers with longer, tubular petals may be better suited for pollinators with longer proboscises, such as hummingbirds or butterflies. Petal size and shape can also affect the landing and feeding behavior of pollinators, influencing their ability to obtain nectar or pollen from the flower. In some aspects, a genetically modified plant of the instant disclosure comprises a genetic modification that modifies expression of a polynucleotide encoding a large petal phenotype.

The size and shape of petals in plants can be determined by various genetic factors that regulate cell division, cell expansion, and organ growth. During flower development, many proteins interact as components of the molecular networks that control biological processes such as the floral meristem emergence and the acquisition of flower organ identity. Once a plant has been induced to flower, the floral organs (sepals, petals, stamens and carpels) develop sequentially on the flanks of the floral meristem under the combinatory action of four classes of organ identity genes (A, B, C and E) following the ABCE model. In this model, A+E specify sepals, A+B+E petals, B+C+E stamens and C+E carpels. These homeotic genes are master regulatory genes that trigger the developmental programs required for flower organogenesis. In Arabidopsis thaliana, these genes are APETALA1 (AP1) and APETALA2 (AP2) for the A class, PISTILLATA (PI) and APETALA3 (AP3) for the B class; AGAMOUS (AG), the only known class C gene and SEPALLATA 1, 2, 3, 4 (SEP1, SEP2, SEP3, SEP4) for the E class. All of these genes (except for AP2) encode MADS-box transcription factors (TFs) that have been proposed to interact and form four different tetrameric complexes according to the ‘quartet model’. This directly links the action of these four different tetrameric complexes to floral organ identity. AP3, PI, SEP3 and either AP1 or AG have been shown to interact to form multimeric protein complexes required to specify petal (in whorl 2) or stamen (in whorl 3) identity, respectively.

### A. ANT Gene

In some aspects, the polynucleotide encoding a large petal phenotype encodes an organ identity genes (A, B, C and E) of the ABCE model, or any combination thereof. In some aspects, the polynucleotide encoding a large petal phenotype encodes an APETALA1 (AP1) protein, an APETALA2 (AP2) protein, a PISTILLATA (PI) protein, and APETALA3 (AP3) protein, an AGAMOUS (AG) protein, a SEPALLATA 1, 2, 3, or 4 (SEP1, SEP2, SEP3, or SEP4) protein, or any combination thereof.

In some aspects, the polynucleotide encoding a large petal phenotype encodes an APETALA2 (APT2)-like ethylene-responsive transcription factor (ANT) protein. ANT protein is a transcription factor involved in the regulation of gene expression in response to ethylene, a plant hormone that plays a critical role in plant growth, development, and stress responses. The ANT protein belongs to the AP2/ERF (APETALA2/ethylene-responsive element binding factor) family of transcription factors, which are known to be involved in regulating gene expression in response to various environmental stimuli, including ethylene. The AP2/ERF family is one of the largest transcription factor families in plants, and members of this family have diverse roles in plant development and stress responses. The ANT protein specifically functions in the regulation of plant growth and development, including embryogenesis, organ development, and flowering. ANT is known to play a role in controlling cell proliferation and differentiation, as well as modulating hormone signaling pathways, such as auxin and gibberellin. In addition to its developmental roles, ANT also plays a role in plant stress responses. It has been implicated in regulating plant responses to various abiotic stresses, such as drought, cold, and salt stress. Its diverse functions make it a key player in the complex regulatory network that governs plant responses to environmental cues.

In some aspects, the introduced genetic modification increases expression of ANT. In some aspects, the genetic modification comprises a nucleic acid sequence in a polynucleotide encoding a programmable transcription regulation system comprising a programmable targeting protein modified for transcriptional regulation of the polynucleotide encoding ANT. Programmable transcription regulation systems are recognized in the art and can be as described in Section IV herein below. In some aspects, the programmable transcription regulation system comprises a CRISPR/Cas nuclease system modified for transcriptional regulation of a locus. In some aspects, the system comprising dCAS9 fused to a transcriptional activator, and a guide RNA (gRNA). In some aspects, the engineered protein comprises dCas9 fused at its C terminus to a polypeptide comprising six copies of TALE TAD (Tail-TV CT engineered protein) fused at its C terminus to VP128 (dCas9-TV) and the gRNA comprises a sequence which binds to a polynucleotide sequence in a promoter region of the polynucleotide encoding ANT, to thereby target the engineered protein to target sequence in the polynucleotide encoding ANT.

In some aspects, the dCas9 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24. In some aspects, the dCas9 protein comprises an amino acid sequence comprising at least about 75% or more, at least about 85% or more, at least about 95% or more, or 100% sequence identity with an amino acid sequence of SEQ ID NO: 24.

In some aspects, the polypeptide comprising six copies of TALE TAD (TAL) comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 48. In some aspects, the polypeptide comprising six copies of TALE TAD (TAL) comprises at least about 75% or more, at least about 85% or more, at least about 95% or more, or 100% sequence identity with an amino acid sequence of SEQ ID NO: 48.

In some aspects, the dCas9-TV protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 9. In some aspects, the dCas9-TV protein comprises an amino acid sequence comprising at least about 75% or more, at least about 85% or more, at least about 95% or more, or 100% sequence identity with an amino acid sequence of SEQ ID NO: 9.

In some aspects, the gRNA comprises a nucleic acid sequence of SEQ ID NO: 1, SEQ ID NO: 2, SEQ ID NO: 3; or any combination thereof. In some aspects, the target of the gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4. In some aspects, the target of the gRNA is a nucleic acid sequence in a polynucleotide comprising at least about 75% or more, at least about 85% or more, at least about 95% or more, or 100% sequence identity with a nucleic acid sequence of SEQ ID NO: 4.

In other aspects, gRNA comprises a nucleic acid sequence of SEQ ID NO: 5, SEQ ID NO: 6, SEQ ID NO: 7, or any combination thereof. In some aspects, the target of the gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 8. In some aspects, the target of the gRNA is a nucleic acid sequence in a polynucleotide comprising at least about 75% or more, at least about 85% or more, at least about 95% or more, or 100% sequence identity with a nucleic acid sequence of SEQ ID NO: 8.

In yet other aspects, the gRNA comprises a nucleic acid sequence of SEQ ID NO: 1, SEQ ID NO: 2, SEQ ID NO: 3, SEQ ID NO: 5, SEQ ID NO: 6, SEQ ID NO: 7, or any combination thereof. In some aspects, the target of the gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4 or SEQ ID NO: 8. In some aspects, the target of the gRNA is a nucleic acid sequence in a polynucleotide comprising at least about 75% or more, at least about 85% or more, at least about 95% or more, or 100% sequence identity with a nucleic acid sequence of SEQ ID NO: 4 or SEQ ID NO: 8.

In some aspects, the polynucleotide encoding the genetic modification of the at least one target site in a polynucleotide encoding ANT comprises a promoter operably linked to a polynucleotide encoding the programmable transcription regulation system. Promoters can be as described in Section V herein below. In some aspects, the promoter directs expression of the polynucleotide encoding ANT in flower cells. In some aspects, the promoter is an AP3 promoter. In some aspect, the promoter is a pU10 promoter.

In some aspects, the AP3 promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 36. In some aspects, the AP3 promoter comprising a nucleic acid sequence comprising at least about 75% or more, at least about 85% or more, at least about 95% or more, or 100% sequence identity with a nucleic acid sequence of SEQ ID NO: 36.

In some aspects, the pU10 promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 30. In some aspects, the pU10 promoter comprising a nucleic acid sequence comprising at least about 75% or more, at least about 85% or more, at least about 95% or more, or 100% sequence identity with a nucleic acid sequence of SEQ ID NO: 30.

In some aspects, the expression construct comprises an AP3 promoter operably linked to a polynucleotide encoding dCas9-TV. In some aspects, the polynucleotide encoding dCas9-TV comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 66. In some aspects, the polynucleotide encoding dCas9-TV comprises at least about 75% or more, at least about 85% or more, at least about 95% or more, or 100% sequence identity with a nucleic acid sequence of SEQ ID NO: 66.

In some aspects, the expression construct comprises an AP3 promoter operably linked to a polynucleotide encoding dCas9-TV. In some aspects, the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 62. In some aspects, the expression construct comprises a nucleic acid sequence comprising at least about 75% or more, at least about 85% or more, at least about 95% or more, or 100% sequence identity with a nucleic acid sequence of SEQ ID NO: 62.

In some aspects, the polynucleotide encoding the programmable transcription regulation system comprises an expression construct for expressing the gRNA.

In some aspects, the polynucleotide encoding the programmable transcription regulation system comprises one or more expression constructs for expressing the gRNA in flower cells. In some aspects, the one or more expression constructs for expressing a gRNA in flower cells comprises a constitutive promoter operably linked to a polynucleotide encoding the gRNA. In some aspects, the expression construct comprises a pU10 promoter operably linked to a polynucleotide encoding the gRNA.

In some aspects, a gRNA comprising a sequence which binds to a polynucleotide sequence in a promoter region of the polynucleotide encoding ANT comprises a nucleic acid sequence of SEQ ID NO: 1, SEQ ID NO: 2, SEQ ID NO: 3; or any combination thereof. In some aspects, a gRNA comprising a sequence which binds to a nucleic acid sequence in a polynucleotide sequence in a promoter region of the polynucleotide encoding ANT comprises a nucleic acid sequence of SEQ ID NO: 5, SEQ ID NO: 6, or SEQ ID NO: 7, or any combination thereof. In some aspects, a gRNA comprising a sequence which binds to a polynucleotide sequence in a promoter region of the polynucleotide encoding ANT comprises a nucleic acid sequence of SEQ ID NO: 1, SEQ ID NO: 2, SEQ ID NO: 3, SEQ ID NO: 5, SEQ ID NO: 6, and SEQ ID NO: 7, or any combination thereof.

In some aspects, the target of a gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4. In some aspects, the target is a nucleic acid sequence in a polynucleotide comprising at least about 75% or more, at least about 85% or more, at least about 95% or more, or 100% sequence identity with a nucleic acid sequence of SEQ ID NO: 4.

In some aspects, the target of a gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 8. In some aspects, the target is a nucleic acid sequence in a polynucleotide comprising at least about 75% or more, at least about 85% or more, at least about 95% or more, or 100% sequence identity with a nucleic acid sequence of SEQ ID NO: 8.

In some aspects, the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4 or SEQ ID NO: 8. In some aspects, the target is a nucleic acid sequence in a polynucleotide comprising at least about 75% or more, at least about 85% or more, at least about 95% or more, or 100% sequence identity with a nucleic acid sequence of SEQ ID NO: 4 or SEQ ID NO: 8.

In some aspects, the gRNA comprises a nucleic acid sequence of SEQ ID NO: 1, SEQ ID NO: 2, SEQ ID NO: 3; or any combination thereof, wherein the target is a nucleic acid sequence in a polynucleotide comprising at least about 75%, 76%, 77%, 93%, 94%, 95%, 96%, 97%, 98%, 99%, or 100% sequence identity with a nucleic acid sequence of SEQ ID NO: 4. In some aspects, the gRNA comprises a nucleic acid sequence of SEQ ID NO: 1, SEQ ID NO: 2, SEQ ID NO: 3; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 4.

In some aspects, the gRNA comprises a nucleic acid sequence of SEQ ID NO: 5, SEQ ID NO: 6, or SEQ ID NO: 7; or any combination thereof, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 8. In some aspects, the gRNA comprises a nucleic acid sequence of SEQ ID NO: 5, SEQ ID NO: 6, or SEQ ID NO: 7; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 8.

In some aspects, the gRNA comprises a nucleic acid sequence of SEQ ID NO: 1, SEQ ID NO: 2, SEQ ID NO: 3, SEQ ID NO: 5, SEQ ID NO: 6, and SEQ ID NO: 7; or any combination thereof, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4 or SEQ ID NO: 8. In some aspects, the gRNA comprises a nucleic acid sequence of SEQ ID NO: 1, SEQ ID NO: 2, SEQ ID NO: 3, SEQ ID NO: 5, SEQ ID NO: 6, and SEQ ID NO: 7; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 4 or SEQ ID NO: 8.

In some aspects, a polynucleotide encoding the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 76. In some aspects, a polynucleotide encoding the gRNA comprises a nucleic acid sequence comprising at least about 75% or more, at least about 85% or more, at least about 95% or more, or 100% sequence identity with a nucleic acid sequence of SEQ ID NO: 76.

In some aspects, an expression construct expressing the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 65. In some aspects, an expression construct expressing the gRNA comprises a nucleic acid sequence comprising at least about 75% or more, at least about 85% or more, at least about 95% or more, or 100% sequence identity with a nucleic acid sequence of SEQ ID NO: 65.

In some aspects, an expression construct comprising a polynucleotide encoding the gRNA comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 67. In some aspects, a polynucleotide encoding the gRNA comprises at least about 75% or more, at least about 85% or more, at least about 95% or more, or 100% sequence identity with a nucleic acid sequence of SEQ ID NO: 67.

In some aspects, the genetically modified plant comprises an expression construct comprising an AP3 promoter operably linked to a polynucleotide encoding dCas9-TV, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 62; an expression construct comprising a pU10 promoter operably linked to the gRNA, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 65 and an expression construct comprising a pU10 promoter operably linked to the gRNA, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 67.

In some aspects, the genetically modified plant comprises an expression construct comprising an AP3 promoter operably linked to a polynucleotide encoding dCas9-TV, wherein the expression construct comprises at least about 75% or more, at least about 85% or more, at least about 95% or more, or 100% sequence identity with a nucleic acid sequence of SEQ ID NO: 62; an expression construct comprising a pU10 promoter operably linked to the gRNA, wherein the expression construct comprises at least about 75% or more, at least about 85% or more, at least about 95% or more, or 100% sequence identity with a nucleic acid sequence of SEQ ID NO: 65 and an expression construct comprising a pU10 promoter operably linked to the gRNA, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 67.

### B. BIGPETALp (BPEp) Protein

In some aspects, the polynucleotide encoding a large petal phenotype is encoded by a realizator protein. In whorl 2, PI, AP3, AP1 and SEP can regulate a set of downstream structural genes (so called ‘realizators’) that encode proteins required for the cell division and differentiation events that lead to petal organogenesis. These downstream proteins must function in a timely, coordinated way, leading to a constant final organ shape and size. Non-limiting examples of proteins encoded by realizator genes include NAP, whose expression is controlled by AP3/PI, has been suggested to function in the transition between growth by cell division and cell expansion in petals and stamens; RABBIT EARS, whose expression is under the control of AP1, is involved in second whorl organ development; PETAL LOSS has been shown to be involved in petal organ initiation and orientation; BIG BROTHER (BB), an E3 ubiquitin ligase-encoding gene, has been shown to limit plant organ size by controlling cell proliferation; BIGPETAL or BPEp; or any combination thereof.

In some aspects, the polynucleotide encoding a large petal phenotype encodes a BIGPETALp protein (BPEp). BPEp is a basic helix-loop-helix encoding gene (BIGPETAL, BPE) involved in the control of petal size in Arabidopsis thalian. In A. thaliana, BPE is expressed via two mRNAs derived from an alternative splicing event. The BPEub (AT1G59640.1) transcript is expressed ubiquitously, whereas the BPEp (AT1G59640.2) transcript is preferentially expressed in petals. Plants that lack the petal-expressed variant BPEp have larger petals as a result of increased cell size. BPEp is positively regulated downstream of APETALA3, PISTILLATA, APETALA1 and PISTILLATA3 and is negatively regulated downstream of AGAMOUS.

In some aspects, the introduced genetic modification reduces expression of BPEp. In some aspects, the genetic modification comprises a polynucleotide encoding a programmable transcription regulation system comprising a programmable targeting protein modified for transcriptional regulation of the polynucleotide encoding BPEp. Programmable transcription regulation systems are recognized in the art and can be as described in Section IV herein below. In some aspects, the programmable transcription regulation system comprises a CRISPR/Cas nuclease system modified for transcriptional regulation of a locus. In some aspects, the programmable transcription regulation system comprises a CRISPR/Cas nuclease system comprising dCAS9 fused to a transcriptional repressor, and a guide RNA (gRNA). In some aspects, the engineered protein comprises dCas9 fused at its C terminus to a polypeptide comprising an EAR-repression domain (SRDX) (dCas9-SRDX) and the gRNA comprises a sequence which binds to a polynucleotide sequence in a promoter region of the polynucleotide encoding BPEp, to thereby target the engineered protein to target sequence in the polynucleotide encoding BPEp, to thereby target the engineered protein to target sequence in the polynucleotide encoding BPEp. In some aspects, the dCas9 protein can be as described in Section II (a) herein above.

In some aspects, the polypeptide comprising an EAR-repression domain (SRDX) comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 46. In some aspects, the polypeptide comprising an EAR-repression domain (SRDX) comprises at least about 75% or more, at least about 85% or more, at least about 95% or more, or 100% sequence identity with an amino acid sequence of SEQ ID NO: 46.

In some aspects, the dCas9 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24 and SRDX comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 46. In some aspects, the dCas9 protein comprises an amino acid sequence comprising at least about 75% or more, at least about 85% or more, at least about 95% or more, or 100% sequence identity with an amino acid sequence of SEQ ID NO: 24 and SRDX comprises at least about 75% or more, at least about 85% or more, at least about 95% or more, or 100% sequence identity with an amino acid sequence of SEQ ID NO: 46.

In some aspects, the engineered protein comprising dCas9 fused at its C terminus to a polypeptide comprising an EAR-repression domain (SRDX) (dCas9-SRDX) comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 10. In some aspects, the engineered protein comprising dCas9 fused at its C terminus to a polypeptide comprising an EAR-repression domain (SRDX) (dCas9-SRDX) comprises an amino acid sequence comprising at least about 75% or more, at least about 85% or more, at least about 95% or more, or 100% sequence identity with an amino acid sequence of SEQ ID NO: 10.

In some aspects, the engineered protein comprising dCas9 fused at its C terminus to a polypeptide comprising an EAR-repression domain (SRDX) (dCas9-SRDX) comprises an amino acid sequence encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 68. In some aspects, the engineered protein comprising dCas9 fused at its C terminus to a polypeptide comprising an EAR-repression domain (SRDX) (dCas9-SRDX) comprises an amino acid sequence encoded by a nucleic acid sequence comprising at least about 75% or more, at least about 85% or more, at least about 95% or more, or 100% sequence identity with a nucleic acid sequence of SEQ ID NO: 68.

In some aspects, the gRNA comprises a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof. In some aspects, the target of the gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 17 or SEQ ID NO: 18. In some aspects, the target of the gRNA is a nucleic acid sequence in a polynucleotide comprising at least about 75% or more, at least about 85% or more, at least about 95% or more, or 100% sequence identity with a nucleic acid sequence of SEQ ID NO: 17 or SEQ ID NO: 18.

In some aspects, the polynucleotide encoding the genetic modification of the at least one target site in a polynucleotide encoding BPEp comprises a promoter operably linked to a polynucleotide encoding the programmable transcription regulation system. In some aspects, the promoter directs expression of the polynucleotide encoding the programmable transcription regulation system in flower cells. Promoters can be as described in Section V herein below. In some aspects, the promoter is an AP3 promoter. In some aspect, the promoter is a pU10 promoter. pAP3 and pU10 can be as described in Section II (a) herein above.

In some aspects, the polynucleotide encoding the programmable transcription regulation system comprises an expression construct for expressing dCas9-SRDX in flower cells and one or more expression constructs for expressing the gRNA in flower cells. In some aspects, the expression construct for expressing dCas9-SRDX in flower cells comprises a flower cell-specific promoter operably linked to a polynucleotide encoding dCas9-SRDX. In some aspects, the expression construct comprises an AP3 promoter operably linked to a polynucleotide encoding dCas9-SRDX. dCas9 can be as described in Section II (a) herein above.

In some aspects, the polynucleotide encoding SRDX comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 45. In some aspects, the polynucleotide encoding dCas9-SRDX comprises at least about 75% or more, at least about 85% or more, at least about 95% or more, or 100% sequence identity with a nucleic acid sequence of SEQ ID NO: 45.

In some aspects, the expression construct comprises an AP3 promoter operably linked to a polynucleotide encoding dCas9-SRDX comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 64. In some aspects, the expression construct an AP3 promoter operably linked to a polynucleotide encoding dCas9-SRDX comprises at least about 75% or more, at least about 85% or more, at least about 95% or more, or 100% sequence identity with a nucleic acid sequence of SEQ ID NO: 64.

In some aspects, the polynucleotide encoding the programmable transcription regulation system comprises an expression construct for expressing the gRNA.

In some aspects, the polynucleotide encoding the programmable transcription regulation system comprises one or more expression constructs for expressing the gRNA in flower cells. In some aspects, the one or more expression constructs for expressing a gRNA in flower cells comprises a constitutive promoter operably linked to a polynucleotide encoding the gRNA. In some aspects, the expression construct comprises a pU10 promoter operably linked to a polynucleotide encoding the gRNA. The pU10 promoter can be as described in Section II (a) herein above.

In some aspects, a gRNA comprising a sequence which binds to a polynucleotide sequence in a promoter region of the polynucleotide encoding BPEp comprises a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof.

In some aspects, the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 17 or SEQ ID NO: 18. In some aspects, the target is a nucleic acid sequence in a polynucleotide comprising at least about 75% or more, at least about 85% or more, at least about 95% or more, or 100% sequence identity with a nucleic acid sequence of SEQ ID NO: 17 or SEQ ID NO: 18.

In some aspects, the gRNA comprises a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 17 or SEQ ID NO: 18. In some aspects, the gRNA comprises a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 17 or SEQ ID NO: 18.

In some aspects, a polynucleotide encoding the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 74. In some aspects, a polynucleotide encoding the gRNA comprises a nucleic acid sequence comprising at least about 75% or more, at least about 85% or more, at least about 95% or more, or 100% sequence identity with a nucleic acid sequence of SEQ ID NO: 74.

In some aspects, the genetically modified plant comprises an expression construct comprising an AP3 promoter operably linked to a polynucleotide encoding dCas9-SRDX, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 64; an expression construct comprising a pU10 promoter operably linked to the gRNA, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 70. In some aspects, the genetically modified plant comprises an expression construct comprising an AP3 promoter operably linked to a polynucleotide encoding dCas9-SRDX, wherein the expression construct comprises at least about 75% or more, at least about 85% or more, at least about 95% or more, or 100% sequence identity with a nucleic acid sequence of SEQ ID NO: 64 and an expression construct comprising a pU10 promoter operably linked to the gRNA, wherein the expression construct comprises at least about 75% or more, at least about 85% or more, at least about 95% or more, or 100% sequence identity with a nucleic acid sequence of SEQ ID NO: 70.

### C. ANT and BPEp

In some aspects, a genetically modified plant of the instant disclosure comprises a genetic modification in a nucleic acid encoding a large petal phenotype, wherein the nucleic acid encoding a large petal phenotype encodes an ANT protein and a BPEp protein. The genetic modifications can be as described in Sections II(b)(A) and II(a)(B).

### (b) Nectar

In some aspects, genetically modified plants of the instant disclosure comprise a genetic modification in a polynucleotide encoding a nectar production phenotype, a nectar quality phenotype, or both. Nectar production and quality in plants involve complex genetic regulation to attract pollinators and ensure successful pollination.

In some aspects, genetically modified plants of the instant disclosure comprise a genetic modification in a polynucleotide encoding a nectar quality phenotype. Nectar quality can include nectar odor, sugar content, other nutrients, or any combination thereof. Specific genes and mechanisms governing nectar production and quality can and will vary among plant species and include genes encoding sucrose transporter proteins; sucrose synthase (SUS) proteins; invertase proteins-responsible for the hydrolysis of sugars where different forms of invertases, such as cell-wall invertases and vacuolar invertases, have been implicated in regulating the sweetness of nectar by affecting the balance between glucose and fructose levels in nectar; phenylalanine ammonia lyase proteins (PAL)-involved in the biosynthesis of phenolic compounds, which are often found in nectar and can affect nectar quality; aromatic amino acid decarboxylase (AAAD)-involved in the biosynthesis of volatile compounds, which can contribute to the aroma and flavor of nectar; and pathogenesis-related genes (PR) genes-involved in plant defense against pathogens, and have been shown to be upregulated in nectar-secreting tissues.

In some aspects, genetically modified plants of the instant disclosure comprise a genetic modification in a polynucleotide encoding a sweet nectar phenotype. The sweetness of nectar in plants is primarily determined by the types and concentrations of sugars present in the nectar controlled by genes encoding sugar transporters, sucrose synthases, and invertases, among other functions. In some aspects, genetically modified plants of the instant disclosure comprise a genetic modification in a polynucleotide encoding a sugar transporter. These transporters are responsible for moving sucrose, the main sugar found in nectar, into and out of nectar-secreting cells. Sucrose transporters can regulate the sugar composition and concentration of nectar, which can affect nectar quality and attractiveness to pollinators. Non-limiting examples of sugar transporters include SUS proteins, invertases, SWEET (Sugars Will Eventually be Exported Transporters); SUT (Sucrose Transporter); TMT (Tonoplast Monosaccharide Transporter); and sucrose-proton symporter proteins.

In some aspects, the polynucleotide encoding a sweet nectar phenotype encodes a sucrose-proton symporter 9 (SUC9) sucrose transporter. In some aspects, the introduced genetic modification increases expression of SUC9. In some aspects, the genetic modification comprises a polynucleotide encoding a programmable transcription regulation system comprising a programmable targeting protein modified for transcriptional regulation of the polynucleotide encoding SUC9. Programmable transcription regulation systems are recognized in the art and can be as described in Section IV herein below. In some aspects, the programmable transcription regulation system comprises a CRISPR/Cas nuclease system modified for transcriptional regulation of a locus. In some aspects, the programmable transcription regulation system comprises a CRISPR/Cas nuclease system comprising dCAS9 fused to a transcriptional activator, and a guide RNA (gRNA). In some aspects, the engineered protein comprises dCas9-TV and the gRNA comprises a sequence which binds to a polynucleotide sequence in a promoter region of the polynucleotide encoding SUC9, to thereby target the engineered protein to target sequence in the polynucleotide encoding SUC9. A dCas9-TV protein can be as described in Section II (a) herein above.

In some aspects, the gRNA comprises a nucleic acid sequence of SEQ ID NO: 20, SEQ ID NO: 21, SEQ ID NO: 22, or any combination thereof. In some aspects, the target of the gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 26. In some aspects, the target of the gRNA is a nucleic acid sequence in a polynucleotide comprising at least about 75% or more, at least about 85% or more, at least about 95% or more, or 100% sequence identity with a nucleic acid sequence of SEQ ID NO: 26.

In other aspects, gRNA comprises a nucleic acid sequence of SEQ ID NO: 27, SEQ ID NO: 28, SEQ ID NO: 29, or any combination thereof. In some aspects, the target of the gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 33. In some aspects, the target of the gRNA is a nucleic acid sequence in a polynucleotide comprising at least about 75% or more, at least about 85% or more, at least about 95% or more, or 100% sequence identity with a nucleic acid sequence of SEQ ID NO: 33.

In yet other aspects, the gRNA comprises a nucleic acid sequence of any combination of SEQ ID NO: 20, SEQ ID NO: 21, SEQ ID NO: 22, SEQ ID NO: 27, SEQ ID NO: 28, or SEQ ID NO: 29. In some aspects, the target of the gRNA is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 26 or SEQ ID NO: 33. In some aspects, the target of the gRNA is a nucleic acid sequence in a polynucleotide at least about 75% or more, at least about 85% or more, at least about 95% or more, or 100% sequence identity with a nucleic acid sequence of SEQ ID NO: 26 or SEQ ID NO: 33.

In some aspects, the polynucleotide encoding the genetic modification of the at least one target site in a polynucleotide encoding SUC9 comprises a promoter operably linked to a polynucleotide encoding the programmable transcription regulation system. Promoters can be as described in Section V herein below. In some aspects, the promoter directs expression of the polynucleotide encoding SUC9 in flower cells. In some aspects, the promoter is an pAGL1 promoter. In some aspect, the promoter is a pU10 promoter. A pU10 promoter can be as described in Section II (a) herein above.

In some aspects, the pAGL1 promoter comprising a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 19. In some aspects, the pAGL1 promoter comprising a nucleic acid sequence comprising at least about 75% or more, at least about 85% or more, at least about 95% or more, or 100% sequence identity with a nucleic acid sequence of SEQ ID NO: 19.

In some aspects, the expression construct comprising a pAGL1 promoter operably linked to a polynucleotide encoding dCas9-TV comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 63. In some aspects, the expression construct an pAGL1 promoter operably linked to a polynucleotide encoding dCas9-TV comprises at least about 75% or more, at least about 85% or more, at least about 95% or more, or 100% sequence identity with a nucleic acid sequence of SEQ ID NO: 63.

In some aspects, the polynucleotide encoding the programmable transcription regulation system comprises one or more expression constructs for expressing the gRNA in flower cells. In some aspects, the one or more expression constructs for expressing a gRNA in flower cells comprise a constitutive promoter operably linked to a polynucleotide encoding the gRNA. In some aspects, the expression construct comprises a pU10 promoter operably linked to a polynucleotide encoding the gRNA. In some aspects, the pU10 can be as described in Section II (a) herein above.

In some aspects, a gRNA comprising a sequence which binds to a polynucleotide sequence in a promoter region of the polynucleotide encoding SUC9 comprises a nucleic acid sequence of SEQ ID NO: 20, SEQ ID NO: 21, SEQ ID NO: 22, or any combination thereof. In some aspects, a gRNA comprising a sequence which binds to a polynucleotide sequence in a promoter region of the polynucleotide encoding ANT comprises a nucleic acid sequence of SEQ ID NO: 27, SEQ ID NO: 28, or SEQ ID NO: 29, or any combination thereof. In some aspects, a gRNA comprising a sequence which binds to a polynucleotide sequence in a promoter region of the polynucleotide encoding ANT comprises a nucleic acid sequence of SEQ ID NO: 20, SEQ ID NO: 21, SEQ ID NO: 22, SEQ ID NO: 27, SEQ ID NO: 28, or SEQ ID NO: 29.

In some aspects, the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 26. In some aspects, the target is a nucleic acid sequence in a polynucleotide comprising at least about 75% or more, at least about 85% or more, at least about 95% or more, or 100% sequence identity with a nucleic acid sequence of SEQ ID NO: 26.

In some aspects, the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 33. In some aspects, the target is a nucleic acid sequence in a polynucleotide comprising at least about 75% or more, at least about 85% or more, at least about 95% or more, or 100% sequence identity with a nucleic acid sequence of SEQ ID NO: 33.

In some aspects, the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 26 or SEQ ID NO: 33. In some aspects, the target is a nucleic acid sequence in a polynucleotide comprising at least about 75% or more, at least about 85% or more, at least about 95% or more, or 100% sequence identity with a nucleic acid sequence of SEQ ID NO: 26 or SEQ ID NO: 33.

In some aspects, the gRNA comprises a nucleic acid sequence of SEQ ID NO: 20, SEQ ID NO: 21, SEQ ID NO: 22, or any combination thereof, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 26. In some aspects, the gRNA comprises a nucleic acid sequence of SEQ ID NO: 20, SEQ ID NO: 21, SEQ ID NO: 22, or any combination thereof; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 26.

In some aspects, the gRNA comprises a nucleic acid sequence of SEQ ID NO: 27, SEQ ID NO: 28, or SEQ ID NO: 29, or any combination thereof, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 33. In some aspects, the gRNA comprises a nucleic acid sequence of SEQ ID NO: 27, SEQ ID NO: 28, or SEQ ID NO: 29, or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 33.

In some aspects, the gRNA comprises a nucleic acid sequence of SEQ ID NO: 20, SEQ ID NO: 21, SEQ ID NO: 22, SEQ ID NO: 27, SEQ ID NO: 28, or SEQ ID NO: 29, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 26 or SEQ ID NO: 33. In some aspects, the gRNA comprises a nucleic acid sequence of SEQ ID NO: 20, SEQ ID NO: 21, SEQ ID NO: 22, SEQ ID NO: 27, SEQ ID NO: 28, or SEQ ID NO: 29, wherein the target is a nucleic acid sequence in a polynucleotide comprising at least about 75% or more, at least about 85% or more, at least about 95% or more, or 100% sequence identity with a nucleic acid sequence of SEQ ID NO: 26 or SEQ ID NO: 33.

In some aspects, a polynucleotide encoding the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 77. In some aspects, a polynucleotide encoding the gRNA comprises a nucleic acid sequence comprising at least about 75% or more, at least about 85% or more, at least about 95% or more, or 100% sequence identity with a nucleic acid sequence of SEQ ID NO: 77.

In some aspects, a polynucleotide encoding the gRNA comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 78. In some aspects, a polynucleotide encoding the gRNA comprises a nucleic acid sequence comprising at least about 75% or more, at least about 85% or more, at least about 95% or more, or 100% sequence identity with a nucleic acid sequence of SEQ ID NO: 78.

In some aspects, the genetically modified plant comprises an expression construct comprising a pU10 promoter operably linked to a polynucleotide encoding a gRNA, wherein the expression construct comprises at least about 75%, 76%, 77%, 93%, 94%, 95%, 96%, 97%, 98%, 99%, or 100% sequence identity with a nucleic acid sequence of SEQ ID NO: 71. In some aspects, an expression construct comprising a polynucleotide encoding the gRNA comprises at least about 75% or more, at least about 85% or more, at least about 95% or more, or 100% sequence identity with a nucleic acid sequence of SEQ ID NO: 71.

In some aspects, the genetically modified plant comprises an expression construct comprising a pU10 promoter operably linked to a polynucleotide encoding a gRNA, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 72. In some aspects, a polynucleotide encoding the gRNA comprises at least about 75% or more, at least about 85% or more, at least about 95% or more, or 100% sequence identity with a nucleic acid sequence of SEQ ID NO: 72.

In some aspects, the genetically modified plant comprises an expression construct comprising a pAGL1 promoter operably linked to a polynucleotide encoding dCas9-TV, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 63; an expression construct comprising a pU10 promoter operably linked to the gRNA, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 71; and an expression construct comprising a pU10 promoter operably linked to the gRNA, wherein the expression construct comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 72. In some aspects, the genetically modified plant comprises an expression construct comprising an AP3 promoter operably linked to a polynucleotide encoding dCas9-TV, wherein the expression construct comprises at least about 75% or more, at least about 85% or more, at least about 95% or more, or 100% sequence identity with a nucleic acid sequence of SEQ ID NO: 63; an expression construct comprising a pU10 promoter operably linked to the gRNA, wherein the expression construct comprises at least about 75% or more, at least about 85% or more, at least about 95% or more, or 100% sequence identity with a nucleic acid sequence of SEQ ID NO: 71 and an expression construct comprising a pU10 promoter operably linked to the gRNA, wherein the expression construct comprises at least about 75% or more, at least about 85% or more, at least about 95% or more, or 100% sequence identity with a nucleic acid sequence of SEQ ID NO: 72.

### (c) Flower Color

In some aspects, genetically modified plants of the instant disclosure comprise a genetic modification in a nucleic acid encoding a flower color phenotype. Non-limiting examples of genes encoding flower pigment that can affect flower color include CHALCONE SYNTHASE (CHS)—A gene that is involved in the production of flavonoid pigments, which are responsible for flower color in many plant species. It catalyzes the first step in the biosynthesis of flavonoids, including anthocyanins, which are responsible for red, purple, and blue colors in flowers; DIHYDROFLAVONOL 4-REDUCTASE (DFR)—A gene that is involved in the biosynthesis of anthocyanins, which are responsible for red, purple, and blue colors in flowers. DFR catalyzes a step in the pathway that converts dihydroflavonols to anthocyanins; ANTHOCYANIDIN SYNTHASE (ANS)—A gene that is involved in the biosynthesis of anthocyanins, which are responsible for red, purple, and blue colors in flowers. ANS catalyzes a step in the pathway that converts leucoanthocyanidins to anthocyanidins; UDP-GLUCOSE: FLAVONOID 3-O-GLUCOSYLTRANSFERASE (UFGT)—A gene that is involved in the final step of anthocyanin biosynthesis, catalyzing the transfer of a glucose molecule to anthocyanidins, which contributes to the stability and color of anthocyanins in flowers; FLAVONOID 3′-HYDROXYLASE (F3′H)—A gene that is involved in the modification of flavonoids, including anthocyanins, by adding a hydroxyl group to the B-ring, resulting in changes in flower coloration; PHYTOENE SYNTHASE (PSY)—A gene that is involved in the biosynthesis of carotenoids, which are responsible for yellow, orange, and red colors in flowers. PSY catalyzes the first committed step in the synthesis of carotenoids; MYB TRANSCRIPTION FACTORS-A group of genes that are involved in regulating the expression of genes involved in flower coloration. MYB transcription factors can activate or repress the expression of genes involved in the biosynthesis of pigments, including anthocyanins and carotenoids, and thus play a key role in determining flower color.

In some aspects, the nucleic acid encoding a flower color phenotype encodes a flavonoid 3′5′-hydroxylase (W1) protein. In some aspects, genetically modified plants of the instant disclosure comprise a purple petal phenotype. In some aspects, the genetically modified plant comprises an engineered polynucleotide encoding an allele of flower color gene W1 encoding a purple flower flavonoid 3′5′-hydroxylase (w1). In some aspects, the introduced genetic modification comprises a nucleic acid expression construct comprising a white flower W1 promoter operably linked to a polynucleotide encoding the purple flower w1 protein

In some aspects, the polynucleotide encoding the allele of flower color gene W1 encoding a purple flower flavonoid 3′5′-hydroxylase (w1) comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 35. In some aspects, the polynucleotide encoding the allele of flower color gene W1 encoding a purple flower flavonoid 3′5′-hydroxylase (w1) comprises at least about 75% or more, at least about 85% or more, at least about 95% or more, or 100% sequence identity with the nucleic acid sequence of SEQ ID NO: 35.

In some aspects, the white flower W1 promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 34. In some aspects, the white flower W1 promoter comprises a nucleic acid sequence comprising at least about 75% or more, at least about 85% or more, at least about 95% or more, or 100% sequence identity with the nucleic acid sequence of SEQ ID NO: 34.

In some aspects, an expression construct comprising a white flower W1 promoter operably linked to a polynucleotide encoding polynucleotide encoding the allele of flower color gene W1 encoding a purple flower flavonoid 3′5′-hydroxylase (W1) comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 73. In some aspects, an expression construct comprising a white flower W1 promoter operably linked to a polynucleotide encoding polynucleotide encoding the allele of flower color gene W1 encoding a purple flower flavonoid 3′5′-hydroxylase (W1) comprises at least about 75% or more, at least about 85% or more, at least about 95% or more, or 100% sequence identity with a nucleic acid sequence of SEQ ID NO: 73.

(d) Aspects, of Plants with Improved Plant-Pollinator Phenotype

Yet another aspect of the instant disclosure encompasses a genetically modified legume plant (Fabaceae), part thereof, or plant cell thereof, comprising two or more introduced genetic modifications in polynucleotides encoding plant-pollinator floral phenotypes selected from the group consisting of a purple petal phenotype, a large petal phenotype, a sweet nectar phenotype, and any combination thereof. The genetic modifications can be as described in Sections II(a), II(b), and II(c).

### III. Reversibly Sterile Plants with Improved Plant Pollinator Phenotype (Hybrid System)

One aspect of the instant disclosure encompasses a genetically modified legume plant (Fabaceae) cell, legume plant, part thereof, or legume plant seed thereof, comprising a reversible-sterility phenotype and an improved plant-pollinator floral phenotype that improves attraction of pollinators to the flowers, thereby increasing the rate of cross-pollination of the genetically modified plant. Together, reversible sterility and improved attraction of pollinators to the flowers create a system that blocks self-fertilization while synergistically recruiting bee pollinators to carry out hybrid crosses and produce a scalable hybrid seed production strategy. Reversibly sterile plants can be as described in Section I and plants with improved attraction of pollinators to the flowers can be as described in Section II herein above.

In some aspects, a genetically modified legume plant (Fabaceae), part thereof, or plant cell thereof, of the instant disclosure comprises an introduced genetic modification in a polynucleotide encoding a plant-pollinator floral phenotype selected from the group consisting of a purple petal phenotype, a large petal phenotype, a sweet nectar phenotype, and any combination thereof and a reversible sterility phenotype. In addition to the introduced genetic modification in a polynucleotide encoding a plant-pollinator floral phenotype, the plant comprises a sterility expression construct comprising a promoter operably linked to a sterility polynucleotide encoding a sterility factor; a fertility restorer expression construct comprising a promoter operably linked to a fertility restorer polynucleotide encoding a fertility restorer; or a sterility expression construct and a fertility restorer expression construct. The genetic modification in a polynucleotide encoding a plant-pollinator floral phenotype increases rate of cross-pollination of the genetically modified plant with a second plant when compared to the rate of cross-pollination between wild type plants and expression of the sterility factor in the plant causes the plant to be sterile. Expression of the fertility restorer in the sterile plant comprising the improved floral phenotype neutralizes, blocks, offsets, prevents, or inhibits the sterility factor causing the plant to be fertile. The combination of the improved plant-pollinator floral phenotype and the reversible sterility phenotype create a synergistic cross pollination phenotype.

### IV. Engineered Nucleic Acid Expression Modification System

One aspect of the present disclosure encompasses an engineered nucleic acid expression modification system for modifying expression of a polynucleotide encoding a plant-pollinator floral phenotype. Non-limiting examples of suitable nucleic acid expression modifications system include programmable nucleic acid modification systems, an expression constructs comprising a promoter operably linked to a polynucleotide encoding a polypeptide or polynucleotide, or any combination thereof.

In some aspects, the nucleic acid modification system is an expression construct comprising a promoter operably linked to a polynucleotide encoding a plant-pollinator floral phenotype. Expression constructs can be as described in Section V (b) herein below. In other aspects, the nucleic acid expression modification system is a programmable nucleic acid modification system targeted to a nucleic acid sequence in a polynucleotide encoding the plant-pollinator floral phenotype.

### (a) Programmable Nucleic Acid Modification Systems

As used herein, a “programmable nucleic acid modification system” is a system capable of targeting a nucleic acid sequence in a polynucleotide and modifying the nucleic acid sequence of the polynucleotide or modifying the expression of the polynucleotide. Accordingly, a programmable nucleic acid modification system can be used to alter a polynucleotide or to alter a protein encoded by the nucleic acid sequence. A programmable nucleic acid modification system can also be used to modify the expression of a nucleic acid sequence encoded by the polynucleotide or modify the expression of a polypeptide encoded by the polynucleotide. In the context of a genetic modification of a polynucleotide encoding a floral trait a plant-pollinator phenotype, the programmable nucleic acid modification system specifically targets a nucleic acid sequence in a polynucleotide encoding the plant-pollinator floral phenotype and modifies expression of the floral trait. The programmable nucleic acid modification system can comprise an interfering nucleic acid molecule or a nucleic acid editing system.

### D. RNAi

In some aspects, the programmable expression modification system comprises an interfering nucleic acid (RNAi) molecule having a nucleotide sequence complementary to a target sequence within a gene encoding the polypeptide or polynucleotide used to inhibit expression of the polypeptide or polynucleotide. RNAi molecules generally act by forming a heteroduplex with a target RNA molecule, which is selectively degraded or “knocked down,” hence inactivating the target RNA. Under some conditions, an interfering RNA molecule can also inactivate a target transcript by repressing transcript translation and/or inhibiting transcription. An interfering RNA is more generally said to be “targeted against” a biologically relevant target, such as a protein, when it is targeted against the nucleic acid encoding the target. For example, an interfering RNA molecule has a nucleotide (nt) sequence which is complementary to an endogenous mRNA of a target gene sequence. Thus, given a target gene sequence, an interfering RNA molecule can be prepared which has a nucleotide sequence at least a portion of which is complementary to a target gene sequence. When introduced into cells, the interfering RNA binds to the target mRNA, thereby functionally inactivating the target mRNA and/or leading to degradation of the target mRNA.

Interfering RNA molecules include, inter alia, small interfering RNA (siRNA), microRNA (miRNA), piwi-interacting RNA (piRNA), long non-coding RNAs (long ncRNAs or lncRNAs), and small hairpin RNAs (shRNA). lncRNAs are widely expressed and have key roles in gene regulation. Depending on their localization and their specific interactions with DNA, RNA and proteins, lncRNAs can modulate chromatin function, regulate the assembly and function of membraneless nuclear bodies, alter the stability and translation of cytoplasmic mRNAs, and interfere with signaling pathways. Piwi-interacting RNA (piRNA) is the largest class of small non-coding RNA molecules expressed in animal cells. piRNAs regulate gene expression through interactions with piwi-subfamily Argonaute proteins. SiRNA are double-stranded RNA molecules, preferably about 19-25 nucleotides in length. When transfected into cells, siRNA inhibit the target mRNA transiently until they are also degraded within the cell. MiRNA and siRNA are biochemically and functionally indistinguishable. Both are about the same in nucleotide length with 5′-phosphate and 3′-hydroxyl ends, and assemble into an RNA-induced silencing complex (RISC) to silence specific gene expression. siRNA and miRNA are distinguished based on origin. siRNA is obtained from long double-stranded RNA (dsRNA), while miRNA is derived from the double-stranded region of a 60-70 nt RNA hairpin precursor. Small hairpin RNAs (shRNA) are sequences of RNA, typically about 50-80 base pairs, or about 50, 55, 60, 65, 70, 75, or about 80 base pairs in length, that include a region of internal hybridization forming a stem loop structure consisting of a base-pair region of about 19-29 base pairs of double-strand RNA (the stem) bridged by a region of single-strand RNA (the loop) and a short 3′ overhang. shRNA molecules are processed within the cell to form siRNA which in turn knock down target gene expression. shRNA can be incorporated into plasmid vectors and integrated into genomic DNA for longer-term or stable expression, and thus longer knockdown of the target mRNA.

Interfering nucleic acid molecules can contain RNA bases, non-RNA bases, or a mixture of RNA bases and non-RNA bases. For example, interfering nucleic acid molecules provided herein can be primarily composed of RNA bases but also contain DNA bases or non-naturally occurring nucleotides. The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone.

### E. Programmable Nucleic Acid Editing Systems

In some aspects, the programmable nucleic acid modification system is a programmable nucleic acid editing system. Editing systems generally comprise a programmable nucleic acid binding domain and a nuclease domain to generate a programmable nucleic acid nuclease system or a transcription factor domain to create a programmable transcription regulation system. A programmable nucleic acid binding domain of an editing system is capable of targeting a nucleic acid sequence in a polynucleotide, a nuclease is capable of facilitating modification of the nucleic acid sequence of the polynucleotide, and a transcription factor is capable of regulating transcription and thus expression of the polynucleotide.

Programmable nucleic acid binding domains rely for specificity on the delivery of exogenous protein(s), and/or a guide RNA (gRNA), deadRNA (dRNA), or single guide RNA (sgRNA) having a sequence which binds specifically to a gene sequence of interest. Non-limiting examples of programmable nucleic acid binding domains that can be used in a programmable nucleic acid editing system include, without limit, a programmable nucleic acid domain of RNA-guided clustered regularly interspersed short palindromic repeats (CRISPR)/CRISPR-associated (Cas) (CRISPR/Cas) nuclease systems, a CRISPR/Cpf1 nuclease system, a zinc finger nuclease (ZFN), a transcription activator-like effector (TALE), a meganuclease, and a ribozyme. Other suitable programmable nucleic acid binding domains will be recognized by individuals skilled in the art.

When the programmable nucleic acid modification system comprises more than one component, such as a binding protein, a nuclease, a transcription factor or a guide nucleic acid, the multi-component modification system can be modular, in that the different components can optionally be distributed among two or more nucleic acid constructs as described herein. The system components can be delivered by a plasmid or viral vector or as a synthetic oligonucleotide. More detailed descriptions of non-limiting examples of programmable nucleic acid editing systems can be as described further below. All the systems described herein below can be modified to comprise a transcription factor, a nuclease, or both.

i. CRISPR Nuclease Systems.

The programmable targeting nuclease can be an RNA-guided CRISPR endonuclease system. The CRISPR system comprises a guide RNA or sgRNA to a target sequence at which a protein of the system introduces a double-stranded break in a target nucleic acid sequence, and a CRISPR-associated endonuclease. The gRNA is a short synthetic RNA comprising a sequence necessary for endonuclease binding, and a preselected ~20 nucleotide spacer sequence targeting the sequence of interest in a genomic target. Non-limiting examples of endonucleases include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

The CRISPR nuclease system can be derived from any type of CRISPR system, including a type I (i.e., IA, IB, IC, ID, IE, or IF), type II (i.e., IIA, IIB, or IIC), type III (i.e., IIIA or IIIB), or type V CRISPR system. The CRISPR/Cas system can be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system can be a type II Cas9 protein, a type V Cpf1 protein, or a derivative thereof. In some aspects, the CRISPR/Cas nuclease is Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises an RNA recognition and/or RNA binding domain, which interacts with the guide RNA. A protein of the CRISPR system also comprises at least one nuclease domain having endonuclease activity. For example, a Cas9 protein can comprise a RuvC-like nuclease domain and an HNH-like nuclease domain, and a Cpf1 protein can comprise a RuvC-like domain. A protein of the CRISPR system can also comprise DNA binding domains, helicase domains, RNase domains, protein-protein interaction domains, dimerization domains, as well as other domains.

A protein of the CRISPR system can be associated with guide RNAs (gRNA). The guide RNA can be a single guide RNA (i.e., sgRNA), or can comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA can comprise GN17-20GG). The gRNA can also comprise a scaffold sequence that forms a stem loop structure and a single-stranded region. The scaffold region can be the same in every gRNA. In some aspects, the gRNA can be a single molecule (i.e., sgRNA). In other aspects, the gRNA can be two separate molecules. Those skilled in the art are familiar with gRNA design and construction, e.g., gRNA design tools are available on the internet or from commercial sources.

A CRISPR system can comprise one or more nucleic acid binding domains associated with one or more, or two or more selected guide RNAs used to direct the CRISPR system to one or more, or two or more selected target nucleic acid loci. For instance, a nucleic acid binding domain can be associated with one or more, or two or more selected guide RNAs, each selected guide RNA, when complexed with a nucleic acid binding domain, causing the CRISPR system to localize to the target of the guide RNA.

The programmable targeting nuclease can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, can create a single-stranded break or nick in the target nucleic acid sequence. Alternatively, a CRISPR nickase in combination with a pair of offset gRNAs can create a double-stranded break in the nucleic acid sequence.

A CRISPR nuclease of the system can be converted to a nickase by one or more mutations and/or deletions. For example, a Cas9 nickase can comprise one or more mutations in one of the nuclease domains, wherein the one or more mutations can be D10A, E762A, and/or D986A in the RuvC-like domain, or the one or more mutations can be H840A (or H839A), N854A and/or N863A in the HNH-like domain.

A nuclease of a CRISPR nuclease system can be inactivated to obtain a programmable targeting protein that can be associated with a transcription factor capable of regulating expression of a polynucleotide. For instance, a CRISPR/Cas system can comprise a nuclease-deficient CAS9 protein (dCAS9) fused to a transcriptional activator and a guide RNA (gRNA).

ii. ssDNA-Guided Argonaute Systems.

Alternatively, the programmable targeting nuclease can comprise a single-stranded DNA-guided Argonaute (Ago) protein. Ago proteins are ubiquitously expressed and bind to siRNAs or miRNAs to guide post-transcriptional gene silencing either by destabilization of the mRNA or by translational repression.

Some prokaryotic Agos use single-stranded guide DNAs and create double-stranded breaks in nucleic acid sequences. The ssDNA-guided Ago endonuclease can be associated with a single-stranded guide DNA.

The Ago programmable protein can be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago protein can be Natronobacterium gregoryi Ago (NgAgo). Alternatively, the Ago protein can be Thermus thermophilus Ago (TtAgo). The Ago protein can also be Pyrococcus furiosus (PfAgo).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA can comprise a 5′ phosphate group. Those skilled in the art are familiar with ssDNA oligonucleotide design and construction.

iii. Zinc Finger Programmable Targeting Proteins.

A ZFN programmable nucleic acid binding protein comprises a DNA-binding zinc finger region that can comprise from about two to seven zinc fingers, for example, about four to six zinc fingers, wherein each zinc finger binds three nucleotides. The zinc finger region can be engineered to recognize and bind to any DNA sequence. Zinc finger design tools or algorithms are available on the internet or from commercial sources. The zinc fingers can be linked together using suitable linker sequences. ZFN programmable nucleic acid binding proteins can be linked to a nuclease or a transcription factor to generate a programmable nucleic acid modification system comprising targeting a specific nucleic acid sequence.

iv. Transcription Activator-Like Effector Nuclease Systems.

The programmable targeting protein can also be a transcription activator-like effector (TALE) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays can be engineered via modular protein design to target any DNA sequence of interest. Other transcription activator-like effector nuclease systems can comprise, but are not limited to, the repetitive sequence, transcription activator like effector (RipTAL) system from the bacterial plant pathogenic Ralstonia solanacearum species complex (Rssc). The nuclease domain of TALEs can be any nuclease domain as described above in Section IV (a) (i).

### V. Meganucleases or Rare-Cutting Endonuclease Systems

The programmable targeting nuclease can also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that can be suitable for the instant disclosure include I-SceI, I-Crel, I-DmoI, or variants and combinations thereof. A meganuclease can be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease can recognize a 7-nucleotide sequence, an 8-nucleotide sequence, or longer recognition sequence. Non-limiting examples of rare-cutting endonucleases include NotI, AscI, PacI, AsiSI, SbfI, and FseI.

vi. Endonuclease Domains

Non-limiting examples of endonucleases include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

vii. Transcription Factor Domains

Non-limiting examples of suitable transcription transcriptional activation domains include, without limit, herpes simplex virus VP16 domain, VP64 (which is a tetrameric derivative of VP16), VP160 (i.e., 10×VP16), VP128, p65 activation domain from NFκB, p53 activation domains 1 and 2, heat-shock factor 1 (HSF1) activation domain, MyoD1 activation domain, GCN4 peptide, 10×GCN4, viral R transactivator (Rta), VPR (a fusion of VP64-p65-Rta), p53 activation domains 1 and 2, CREB (CAMP response element binding protein) activation domains, E2A activation domains, activation domains from human heat-shock factor 1 (HSF1), NFAT (nuclear factor of activated T-cells) activation domains, a histone acetyltransferase, activation domains from the Arabidopsis thaliana MYB46, HAM1, HAM2, MYB112, WRKY11, ERF6, or any combination thereof. Engineered transcription activation systems may comprise one transcription activation domain, two transcription activation domains, three transcription activation domains, or more than three transcription activation domains.

Non-limiting examples of transcription repressor domains include Kruppel-associated Box (KRAB), EAR-repression domain (SRDX), and BRD.

viii. Optional Additional Domains.

The programmable targeting nuclease can further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS can be located at the N-terminus, the C-terminal, or in an internal location of the fusion protein.

A cell-penetrating domain can be a cell-penetrating peptide sequence derived from the HIV-1 TAT protein. The cell-penetrating domain can be located at the N-terminus, the C-terminal, or in an internal location of the fusion protein.

A programmable targeting nuclease can further comprise at least one linker. For example, the programmable targeting nuclease, the nuclease domain of the targeting nuclease, and other optional domains can be linked via one or more linkers. The linker can be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13 (5): 3096-312). In alternate aspects, the programmable targeting nuclease, the cell cycle regulated protein, and other optional domains can be linked directly.

A programmable targeting nuclease can further comprise an organelle localization or targeting signal that directs a molecule to a specific organelle. A signal can be a polynucleotide or polypeptide signal, or can be an organic or inorganic compound sufficient to direct an attached molecule to a desired organelle. Organelle localization signals can be as described in U.S. Patent Publication No. 20070196334, the disclosure of which is incorporated herein in its entirety.

### (b) Aspects of Engineered Nucleic Acid Modification Systems

In some aspects, when the engineered nucleic acid modification system is a programmable nucleic acid modification system. In some aspects, the programmable nucleic acid modification system is modified for transcriptional regulation of a locus.

In some aspects, the programmable nucleic acid modification system is a CRISPR/Cas system comprising a Cas9 nuclease and a guide RNA (gRNA) comprising a sequence complementary to a target sequence within a nucleic acid sequence in a polynucleotide encoding the plant-pollinator floral phenotype. In some aspects, the CRISPR/Cas system is modified for transcriptional regulation of a locus. In some aspects, the CRISPR/Cas system comprises a CRISPR/Cas nuclease system, the system comprising a nuclease-deficient CAS9 protein (dCAS9) fused to a transcriptional activator and a guide RNA (gRNA).

The engineered system is a transcription inhibitor. In some aspects, the programmable nucleic acid modification system comprises an engineered protein comprising dCas9 fused at its C terminus to a polypeptide comprising six copies of TALE TAD (TAL) fused at its C terminus to VP128 (dCas9-TV). In some aspects, the dCas9 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24 and the polypeptide comprising six copies of TALE TAD (TAL) fused at its C terminus to VP128 (dCas9-TV) protein comprises at least about 75% or more, at least about 85% or more, at least about 95% or more, or 100% sequence identity with an amino acid sequence of SEQ ID NO: 48. In some aspects, the dCas9 protein comprises an amino acid sequence comprising at least about 75% or more, at least about 85% or more, at least about 95% or more, or 100% sequence identity with an amino acid sequence of SEQ ID NO: 24 and the polypeptide comprising six copies of TALE TAD (TAL) fused at its C terminus to VP128 (dCas9-TV) protein comprises at least about 75% or more, at least about 85% or more, at least about 95% or more, or 100% sequence identity with an amino acid sequence of SEQ ID NO: 48.

The engineered system is a transcription repressor. In some aspects, the programmable nucleic acid modification system comprises an engineered protein comprising dCAS9 fused at its C terminus to a polypeptide comprising an EAR-repression domain (SRDX; dCas9-SRDX). In some aspects, the dCas9 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24 and the polypeptide comprising a polypeptide comprising a polypeptide comprising an EAR-repression domain comprises at least about 75% or more, at least about 85% or more, at least about 95% or more, or 100% sequence identity with an amino acid sequence of SEQ ID NO: 46. In some aspects, the dCas9 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24 and the polypeptide comprising a polypeptide comprising an EAR-repression domain comprises at least about 75% or more, at least about 85% or more, at least about 95% or more, or 100% sequence identity with an amino acid sequence of SEQ ID NO: 46. In some aspects, the dCas9 protein comprises an amino acid sequence comprising at least about 75% or more, at least about 85% or more, at least about 95% or more, or 100% sequence identity with an amino acid sequence of SEQ ID NO: 24 and the polypeptide comprising a polypeptide comprising an EAR-repression domain comprises at least about 75% or more, at least about 85% or more, at least about 95% or more, or 100% sequence identity with an amino acid sequence of SEQ ID NO: 46.

In some aspects, the programmable nucleic acid modification system targets a promoter region of a polynucleotide encoding an AP2-like ethylene-responsive transcription factor (ANT). In some aspects, the promoter region of the polynucleotide encoding an ANT protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4. In some aspects, the promoter region of the polynucleotide encoding an ANT protein comprises a nucleic acid sequence comprising at least about 75% or more, at least about 85% or more, at least about 95% or more, or 100% sequence identity with a nucleic acid sequence of SEQ ID NO: 4. In some aspects, the promoter region of a polynucleotide encoding an ANT protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 8. In some aspects, the promoter region of a polynucleotide encoding an ANT protein comprises a nucleic acid sequence comprising at least about 75% or more, at least about 85% or more, at least about 95% or more, or 100% sequence identity with a nucleic acid sequence of SEQ ID NO: 8.

When the programmable nucleic acid modification system is a nuclease-deficient CAS9 protein (dCAS9) fused to a transcriptional activator and a guide RNA (gRNA) that targets the engineered protein to a nucleic acid sequence in a promoter region of the polynucleotide encoding ANT, the gRNA can comprise a nucleic acid sequence of SEQ ID NO: 1, SEQ ID NO: 2, SEQ ID NO: 3; or any combination thereof, a nucleic acid sequence of SEQ ID NO: 5, SEQ ID NO: 6, or SEQ ID NO: 7, or any combination of SEQ ID NO: 1, SEQ ID NO: 2, SEQ ID NO: 3, SEQ ID NO: 5, SEQ ID NO: 6, and SEQ ID NO: 7.

In some aspects, the nucleic acid modification system comprises a dCas9 protein comprising an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24 and a polypeptide comprising six copies of TALE TAD (TAL) fused at its C terminus to VP128 (dCas9-TV) protein comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 48; and gRNAs comprising a nucleic acid sequence of SEQ ID NO: 1, SEQ ID NO: 2, SEQ ID NO: 3; or any combination thereof, and wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4; a nucleic acid sequence of SEQ ID NO: 5, SEQ ID NO: 6, SEQ ID NO: 7, or any combination thereof, and wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 8; or any combination of SEQ ID NO: 1, SEQ ID NO: 2, SEQ ID NO: 3, SEQ ID NO: 5, SEQ ID NO: 6, and SEQ ID NO: 7. In some aspects, the nucleic acid modification system comprises a dCas9 protein comprising an amino acid sequence comprising at least about 75% or more, at least about 85% or more, at least about 95% or more, or 100% sequence identity with an amino acid sequence of SEQ ID NO: 24 and a polypeptide comprising six copies of TALE TAD (TAL) fused at its C terminus to VP128 (dCas9-TV) protein comprising at least about 75% or more, at least about 85% or more, at least about 95% or more, or 100% sequence identity with an amino acid sequence of SEQ ID NO: 48; and gRNAs comprising a nucleic acid sequence of SEQ ID NO: 1, SEQ ID NO: 2, SEQ ID NO: 3; or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 4; a nucleic acid sequence of SEQ ID NO: 5, SEQ ID NO: 6, SEQ ID NO: 7, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 8; or any combination of SEQ ID NO: 1, SEQ ID NO: 2, SEQ ID NO: 3, SEQ ID NO: 5, SEQ ID NO: 6, and SEQ ID NO: 7.

In some aspects, the programmable nucleic acid modification system targets a nucleic acid sequence in a promoter region of a polynucleotide encoding BIGPETALp (BPEp) protein to thereby target the engineered protein to the polynucleotide encoding BPEp. In some aspects, the promoter region of the polynucleotide encoding BIGPETALp (BPEp) comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 17. In some aspects, the promoter region of the polynucleotide encoding BIGPETALp (BPEp) comprises at least about 75% or more, at least about 85% or more, at least about 95% or more, or 100% sequence identity with a nucleic acid sequence of SEQ ID NO: 17. In some aspects, the promoter region of the polynucleotide encoding BIGPETALp (BPEp) comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 18. In some aspects, the promoter region of the polynucleotide encoding BIGPETALp (BPEp) comprises at least about 75% or more, at least about 85% or more, at least about 95% or more, or 100% sequence identity with a nucleic acid sequence of SEQ ID NO: 18.

When the programmable nucleic acid modification system is a nuclease-deficient CAS9 protein (dCAS9) fused to a transcriptional activator and a guide RNA (gRNA) that targets the engineered protein to a nucleic acid sequence in a promoter region of the polynucleotide encoding BIGPETALp (BPEp), the gRNA comprises a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof.

In some aspects, the nucleic acid modification system comprises a dCas9 protein comprising an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24 and a polypeptide comprising a polypeptide comprising an EAR-repression domain comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 46; and a gRNA comprising a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof.

In some aspects, the nucleic acid modification system comprises a dCas9 protein comprising an amino acid sequence comprising at least about 75% or more, at least about 85% or more, at least about 95% or more, or 100% sequence identity with an amino acid sequence of SEQ ID NO: 24 and a polypeptide comprising a polypeptide comprising an EAR-repression domain comprises at least about 75% or more, at least about 85% or more, at least about 95% or more, or 100% sequence identity with an amino acid sequence of SEQ ID NO: 46; and a gRNA comprising a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof.

In some aspects, the nucleic acid modification system comprises an engineered protein comprising dCAS9 fused at its C terminus to a polypeptide comprising an EAR-repression domain (SRDX; dCas9-SRDX). In some aspects, the dCas9 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24 and the polypeptide comprising a polypeptide comprising an EAR-repression domain comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 46; and gRNAs comprising a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof, and wherein the target is a nucleic acid sequence in a promoter region of the polynucleotide encoding BIGPETALp (BPEp), wherein the promoter region comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 17; at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 18.

In some aspects, the dCas9 protein comprises an amino acid sequence comprising at least about 75% or more, at least about 85% or more, at least about 95% or more, or 100% sequence identity with an amino acid sequence of SEQ ID NO: 24 and the polypeptide comprising a polypeptide comprising an EAR-repression domain comprises at least about 75% or more, at least about 85% or more, at least about 95% or more, or 100% sequence identity with an amino acid sequence of SEQ ID NO: 46; and gRNAs comprising a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof, and wherein the target is a nucleic acid sequence in a promoter region of the polynucleotide encoding BIGPETALp (BPEp), wherein the promoter region comprises at least about 75% or more, at least about 85% or more, at least about 95% or more, or 100% sequence identity with a nucleic acid sequence of SEQ ID NO: 17; at least about 75% or more, at least about 85% or more, at least about 95% or more, or 100% sequence identity with a nucleic acid sequence of SEQ ID NO: 18.

In some aspects, the programmable nucleic acid modification system targets a nucleic acid sequence in a promoter region of a polynucleotide encoding a sucrose-proton symporter 9 (SUC9) sucrose transporter. In some aspects, the promoter region of the polynucleotide encoding a sucrose-proton symporter 9 (SUC9) sucrose transporter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 26. In some aspects, the promoter region of the polynucleotide encoding a sucrose-proton symporter 9 (SUC9) sucrose transporter comprises a nucleic acid sequence comprising at least about 75% or more, at least about 85% or more, at least about 95% or more, or 100% sequence identity with a nucleic acid sequence of SEQ ID NO: 26. In some aspects, the promoter region of the polynucleotide encoding a sucrose-proton symporter 9 (SUC9) sucrose transporter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 33. In some aspects, the promoter region of the polynucleotide encoding a sucrose-proton symporter 9 (SUC9) sucrose transporter comprises a nucleic acid sequence comprising at least about 75% or more, at least about 85% or more, at least about 95% or more, or 100% sequence identity with a nucleic acid sequence of SEQ ID NO: 33.

In some aspects, the programmable nucleic acid modification system comprises an engineered protein comprising dCAS9 fused to TV and the gRNA comprises a sequence which binds to a nucleic acid sequence in a promoter region of a polynucleotide encoding a sucrose-proton symporter 9 (SUC9) sucrose transporter, to thereby target the engineered protein to a nucleic acid sequence in a polynucleotide encoding the SUC9 sucrose transporter.

When the programmable nucleic acid modification system is a nuclease-deficient CAS9 protein (dCAS9) fused to a transcriptional activator and the guide RNA (gRNA) that targets the engineered protein to a nucleic acid sequence in a promoter region of a polynucleotide encoding a sucrose-proton symporter 9 (SUC9) sucrose transporter, the gRNA can comprise a nucleic acid sequence of SEQ ID NO: 20, SEQ ID NO: 21, SEQ ID NO: 22, or any combination thereof, a nucleic acid sequence of SEQ ID NO: 27, SEQ ID NO: 28, or SEQ ID NO: 29, or any combination of SEQ ID NO: 20, SEQ ID NO: 21, SEQ ID NO: 22, SEQ ID NO: 27, SEQ ID NO: 28, and SEQ ID NO: 29.

In some aspects, the nucleic acid modification system comprises an engineered protein comprising dCAS9 fused at its C terminus to a polypeptide comprising an EAR-repression domain (SRDX; dCas9-SRDX). In some aspects, the dCas9 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 24 and the a polypeptide comprising six copies of TALE TAD (TAL) fused at its C terminus to VP128 (dCas9-TV) protein comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 48; the gRNA can comprise a nucleic acid sequence of SEQ ID NO: 20, SEQ ID NO: 21, SEQ ID NO: 22, or any combination thereof, a nucleic acid sequence of SEQ ID NO: 27, SEQ ID NO: 28, or SEQ ID NO: 29, or any combination of SEQ ID NO: 20, SEQ ID NO: 21, SEQ ID NO: 22, SEQ ID NO: 27, SEQ ID NO: 28, or SEQ ID NO: 29, or any combination thereof, and wherein the target is a nucleic acid sequence in a promoter region of the polynucleotide encoding sucrose-proton symporter 9 (SUC9) sucrose transporter, wherein the promoter region comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 26; at least about 75% or more, at least about 85% or more, at least about 95% or more, or 100% sequence identity with a nucleic acid sequence of SEQ ID NO: 33.

In some aspects, the dCas9 protein comprises an amino acid sequence comprising at least about 75% or more, at least about 85% or more, at least about 95% or more, or 100% sequence identity with an amino acid sequence of SEQ ID NO: 24 and the a polypeptide comprising six copies of TALE TAD (TAL) fused at its C terminus to VP128 (dCas9-TV) protein comprising at least about 75% or more, at least about 85% or more, at least about 95% or more, or 100% sequence identity with an amino acid sequence of SEQ ID NO: 48; the gRNA can comprise a nucleic acid sequence of SEQ ID NO: 20, SEQ ID NO: 21, SEQ ID NO: 22, or any combination thereof, a nucleic acid sequence of SEQ ID NO: 27, SEQ ID NO: 28, or SEQ ID NO: 29, or any combination of SEQ ID NO: 20, SEQ ID NO: 21, SEQ ID NO: 22, SEQ ID NO: 27, SEQ ID NO: 28, or SEQ ID NO: 29, or any combination thereof, and wherein the target is a nucleic acid sequence in a promoter region of the polynucleotide encoding sucrose-proton symporter 9 (SUC9) sucrose transporter, wherein the promoter region comprises at least about 75% or more, at least about 85% or more, at least about 95% or more, or 100% sequence identity with a nucleic acid sequence of SEQ ID NO: 26; at least about 75% or more, at least about 85% or more, at least about 95% or more, or 100% sequence identity with a nucleic acid sequence of SEQ ID NO: 33.

In some aspects, the genetic modification is in a polynucleotide encoding a purple petal phenotype. In some aspects, the genetically modified plant comprises a genetic modification in a polynucleotide encoding an allele of flower color gene W1 encoding a purple flower flavonoid 3′5′-hydroxylase (W1). In some aspects, the genetic modification comprises a nucleic acid expression construct comprising a white flower W1 promoter operably linked to a polynucleotide encoding the purple flower W1 protein.

The W1 promoter can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 34. The W1 promoter can comprise a nucleic acid sequence comprising at least about 75% or more, at least about 85% or more, at least about 95% or more, or 100% sequence identity with the nucleic acid sequence of SEQ ID NO: 34. In some aspects, the polynucleotide encoding the W1 protein comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 35. In some aspects, the polynucleotide encoding the W1 protein comprises at least about 75% or more, at least about 85% or more, at least about 95% or more, or 100% sequence identity with the nucleic acid sequence of SEQ ID NO: 35. In some aspects, the W1 promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 34; and the polynucleotide encoding the W1 protein comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 35. In some aspects, the W1 promoter comprises a nucleic acid sequence comprising at least about 75% or more, at least about 85% or more, at least about 95% or more, or 100% sequence identity with the nucleic acid sequence of SEQ ID NO: 34; and the polynucleotide encoding the W1 protein comprises at least about 75% or more, at least about 85% or more, at least about 95% or more, or 100% sequence identity with the nucleic acid sequence of SEQ ID NO: 35

### V. Nucleic Acid Constructs

A further aspect of the present disclosure provides a system of one or more nucleic acid constructs encoding the components of the engineered nucleic acid modification system described in Section II herein above.

Any of the multi-component systems described herein are to be considered modular, in that the different components can optionally be distributed among two or more nucleic acid constructs as described herein. The nucleic acid constructs can be DNA or RNA, linear or circular, single-stranded or double-stranded, or any combination thereof. The nucleic acid constructs can be codon-optimized for efficient translation into protein, and possibly for transcription into an RNA donor polynucleotide transcript in the cell of interest. Codon optimization programs are available as freeware or from commercial sources.

The nucleic acid constructs can be used to express one or more components of the system for later introduction into a cell to be genetically modified. Alternatively, the nucleic acid constructs can be introduced into the cell to be genetically modified for expression of the components of the system in the cell. In some aspects, the nucleic acid constructs transiently express the various components of the system. Transiently expressing the system in a plant overcomes the cumbersome regulatory hurdles required for traditionally genetically modified crops. In some aspects, the engineered nucleic acid modification system is expressed in male reproductive tissues, modifies expression of various factors described herein above in male reproductive tissues, or both.

Expression constructs generally comprise DNA coding sequences operably linked to at least one promoter control sequence for expression in a cell of interest. Promoter control sequences can control expression of the transposase, the programmable targeting nuclease, the donor polynucleotide, or combinations thereof in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. As explained above, methylation of the MeSWEET10a gene can be targeted in leaves by specifically expressing the system in leaves using a leaf-specific promoter, allowing for fine-tuning pathogen resistance and normal plant growth and development.

Suitable eukaryotic constitutive promoter control sequences include, but are not limited to, pU10, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIlb promoter, ICAM-2 promoter, INF-β promoter, Mb promoter, NphsI promoter, OG-2 promoter, SP-B promoter, SYN1 promoter, and WASP promoter.

Promoters can also be plant-specific promoters, or promoters that can be used in plants. A wide variety of plant promoters are known to those of ordinary skill in the art, as are other regulatory elements that can be used alone or in combination with promoters.

Promoters can be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604,121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter can be a promoter which is induced by one or more, but not limited to one of the following: abiotic stresses such as wounding, cold, desiccation, ultraviolet-B, heat shock or other heat stress, drought stress or water stress. The promoter can further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize Ivr2 gene promoter; heat-inducible promoters such as heat tomato hsp80-promoter from tomato.

Tissue-specific promoters can include, but are not limited to, fiber-specific, green tissue-specific, root-specific, stem-specific, flower-specific, callus-specific, pollen-specific, egg-specific, promoters specific to male or female reproductive tissues, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262:12202, 1987; Baszczynski et al., Plant Mol. Biol. 14:633, 1990), Brazil Nut albumin (Pearson et al., Plant Mol. Biol. 18:235-245, 1992), legumin (Ellis et al., Plant Mol. Biol. 10:203-214, 1988), Glutelin (rice) (Takaiwa et al., Mol. Gen. Genet. 208:15-22, 1986; Takaiwa et al., FEBS Letts. 221:43-47, 1987), Zein (Matzke et al., Plant Mol Biol, 143:323-32, 1990), napA (Stalberg et al., Planta 199:515-519, 1996), Wheat SPA (Albanietal, Plant Cell, 9:171-184, 1997), sunflower oleosin (Cummins et al., Plant Mol. Biol. 19:873-876, 1992)], endosperm specific promoters [e.g., wheat LMW and HMW, glutenin-1 (Mol Gen Genet 216:81-90, 1989; NAR 17:461-2), wheat a, b, and g gliadins (EMBO3: 1409-15, 1984), Barley Itrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116 (1): 53-62, 1998), Biz2 (EP99106056.7), Synthetic promoter (Vicente-Carbajosa et al., Plant J. 13:629-640, 1998), rice prolamin NRP33, rice-globulin Glb-1 (Wu et al., Plant Cell Physiology 39 (8) 885-889, 1998), rice alpha-globulin REB/OHP-1 (Nakase et al., Plant Mol. Biol. 33:513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93:8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3].

The reversibly sterile plants of the instant disclosure can be male-sterile or female-sterile. In some aspects, the genetically modified plants of the instant disclosure are male-sterile. In some aspects, male-sterile plants comprise a sterility expression construct comprising a male organ- or tissue-specific promoter operably linked to a sterility factor. Non-limiting examples of male organ- or tissue-specific promoters and include the pTA29 promoter, the pTA26 promoter and the pTA13 promoter, as described in U.S. Pat. No. 5,652,354, the disclosure of which is incorporated herein in its entirety, which have been isolated from tobacco and are tapetum-specific promoters; as well as any promoter of a gene encoding a tapetum-specific mRNA hybridizable to the genes TA29, TA26 or TA13, from which genes the pTA29, pTA26 and pTA13 promoters have been isolated. In some aspects, the male organ- or tissue-specific promoter is a pTA29a version of the pTA29 promoter. Importantly, the inventors unexpectedly discovered that the pTA29b version of the pTA29 promoter cannot be used in a sterility/fertility restorer system of the instant disclosure.

In some aspects, the genetically modified plants of the instant disclosure are female-sterile. In some aspects, female-sterile plants comprise a sterility expression construct comprising a female organ- or tissue-specific promoter operably linked to a sterility factor. Non-limiting examples of female organ- or tissue-specific first promoters and sterility promoters are: the style and/or stigma-specific promoters, such as PSTMG07, PSTMG08, PSTMG4B12 and PSTMG3C9, and the ovule-specific promoter corresponding to the cDNA clone pMON9608 as described in U.S. Pat. No. 5,633,441; as well as a promoter of a gene encoding i) a style-stigma specific or ii) an ovule-specific mRNA hybridizable respectively to i) a STMG-type style-stigma specific gene or ii) CDNA clone pMON9608 of U.S. Pat. No. 5,633,441.

Any of the promoter sequences can be wild type or can be modified for more efficient or efficacious expression. The DNA coding sequence also can be linked to a polyadenylation signal (e.g., SV40 polyA signal, bovine growth hormone (BGH) polyA signal, etc.) and/or at least one transcriptional termination sequence. In some situations, the complex or fusion protein can be purified from the bacterial or eukaryotic cells.

Nucleic acids encoding one or more components of an engineered DNA methylation system and/or transcription activation system can be present in a construct. Suitable constructs include plasmid constructs, viral constructs, and self-replicating RNA (Yoshioka et al., Cell Stem Cell, 2013, 13:246-254). For instance, the nucleic acid encoding one or more components of an engineered DNA methylation system and/or transcription activation system can be present in a plasmid construct.

Non-limiting examples of suitable plasmid constructs include pUC, pBR322, pET, pBluescript, and variants thereof. Alternatively, the nucleic acid encoding one or more components of an engineered DNA methylation system and/or transcription activation system can be part of a viral vector (e.g., lentiviral vectors, adeno-associated viral vectors, adenoviral vectors, and so forth).

The plasmid or viral vector can comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasmid or viral vector can further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector can further comprise sequences for expression of Csy4 RNAse to process the gRNA transcript. Additional information about vectors and use thereof can be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

The plasmid or viral vector can also comprise a transit peptide for targeting of a protein product, particularly to a chloroplast, leucoplast or other plastid organelle or vacuole or an extracellular location. For descriptions of the use of chloroplast transit peptides, see U.S. Pat. Nos. 5,188,642 and 5,728,925, herein incorporated by reference in their entirety. Many chloroplast-localized proteins are expressed from nuclear genes as precursors and are targeted to the chloroplast by a chloroplast transit peptide (CTP). Examples of other such isolated chloroplast proteins include, but are not limited to those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS) and transit peptides described in U.S. Pat. No. 7,193,133, herein incorporated by reference. It has been demonstrated in vivo and in vitro that non-chloroplast proteins can be targeted to the chloroplast by use of protein fusions with a heterologous CTP and that the CTP is sufficient to target a protein to the chloroplast. Incorporation of a suitable chloroplast transit peptide, such as, the Arabidopsis thaliana EPSPS CTP (CTP2, Klee et al., Mol. Gen. Genet. 210:437-442), and the Petunia hybrida EPSPS CTP (CTP4, della-Cioppa et al., Proc. Natl. Acad. Sci. USA 83:6873-6877) has been show to target heterologous EPSPS protein sequences to chloroplasts in transgenic plants. The production of glyphosate tolerant plants by expression of a fusion protein comprising an amino-terminal CTP with a glyphosate resistant EPSPS enzyme is well known by those skilled in the art, (U.S. Pat. Nos. 5,627,061, 5,633,435, 5,312,910, EP 0218571, EP 189707, EP 508909, and EP 924299).

In some aspects, when a sterility expression construct comprises a pTA29b promoter operably linked to a polynucleotide encoding barnase, the sterility expression construct can be incorporated into a plasmid vector. In some aspects, a plasmid vector comprising the sterility construct can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 59 or SEQ ID NO: 102. In some aspects, a plasmid vector comprising the sterility construct can comprise a nucleic acid sequence comprising at least about 75% or more, at least about 85% or more, at least about 95% or more, or 100% sequence identity with a nucleic acid sequence of SEQ ID NO: 59 or SEQ ID NO: 102.

In some aspects, when a fertility restorer expression construct comprises a pTA29a promoter operably linked to a polynucleotide encoding barstar, the fertility restorer expression construct can be incorporated into a plasmid vector. In some aspects, a plasmid vector comprising the fertility restorer construct can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 60 or SEQ ID NO: 101. In some aspects, a plasmid vector comprising the fertility restorer construct can comprise a nucleic acid sequence comprising at least about 75% or more, at least about 85% or more, at least about 95% or more, or 100% sequence identity with a nucleic acid sequence of SEQ ID NO: 60 or SEQ ID NO: 101.

In some aspects, when a fertility restorer expression construct comprises a pTA29b promoter operably linked to a polynucleotide encoding barstar, the fertility restorer expression construct can be incorporated into a plasmid vector. In some aspects, a plasmid vector comprising the fertility restorer construct can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 61 or SEQ ID NO: 100. In some aspects, a plasmid vector comprising the fertility restorer construct can comprise a nucleic acid sequence comprising at least about 75% or more, at least about 85% or more, at least about 95% or more, or 100% sequence identity with a nucleic acid sequence of SEQ ID NO: 60 or SEQ ID NO: 101.

In some aspects, when an expression construct for expressing dCas9-TV comprises an AP3 promoter operably linked to a polynucleotide encoding dCas9-TV, the expression construct can be incorporated into a plasmid vector. In some aspects, a plasmid vector comprising the expression construct for expressing dCas9-TV can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 99. In some aspects, a plasmid vector comprising the expression construct for expressing dCas9-TV can comprise a nucleic acid sequence comprising at least about 75% or more, at least about 85% or more, at least about 95% or more, or 100% sequence identity with a nucleic acid sequence of 99.

In some aspects, when an expression construct for expressing dCas9-TV comprises an AGL1 promoter operably linked to a polynucleotide encoding dCas9-TV, the expression construct can be incorporated into a plasmid vector. In some aspects, a plasmid vector comprising the expression construct for expressing dCas9-TV can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 69. In some aspects, a plasmid vector comprising the expression construct for expressing dCas9-TV can comprise a nucleic acid sequence comprising at least about 75% or more, at least about 85% or more, at least about 95% or more, or 100% sequence identity with a nucleic acid sequence of SEQ ID NO: 69.

In some aspects, when an expression construct for expressing gRNA comprises a pU10 promoter operably linked to a polynucleotide encoding the gRNAs comprising SEQ ID NO: 1, SEQ ID NO: 2, and SEQ ID NO: 3, the expression construct can be incorporated into a plasmid vector. In some aspects, a plasmid vector comprising the expression construct for expressing the gRNAs can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 75. In some aspects, the plasmid vector comprising the expression construct for expressing the gRNAs can comprise a nucleic acid sequence comprising at least about 75% or more, at least about 85% or more, at least about 95% or more, or 100% sequence identity with a nucleic acid sequence of SEQ ID NO: 75.

In some aspects, when an expression construct for expressing gRNA comprises a pU10 promoter operably linked to a polynucleotide encoding the gRNAs comprising SEQ ID NO: 5, SEQ ID NO: 6, or SEQ ID NO: 7, the expression construct can be incorporated into a plasmid vector. In some aspects, a plasmid vector comprising the expression construct for expressing the gRNAs can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 76. In some aspects, the plasmid vector comprising the expression construct for expressing the gRNAs can comprise a nucleic acid sequence comprising at least about 75% or more, at least about 85% or more, at least about 95% or more, or 100% sequence identity with a nucleic acid sequence of SEQ ID NO: 76.

In some aspects, when an expression construct for expressing W1 comprises a white flower W1 promoter operably linked to a polynucleotide encoding polynucleotide encoding the allele of flower color gene W1 encoding a purple flower flavonoid 3′5′-hydroxylase (W1), the expression construct can be incorporated into a plasmid vector. In some aspects, a plasmid vector comprising the expression construct for expressing W1 can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 103. In some aspects, the plasmid vector comprising the expression construct for expressing W1 can comprise a nucleic acid sequence comprising at least about 75% or more, at least about 85% or more, at least about 95% or more, or 100% sequence identity with a nucleic acid sequence of SEQ ID NO: 103.

In some aspects, when a programmable nucleic acid modification system targets a nucleic acid sequence in a promoter region of a polynucleotide encoding a sucrose-proton symporter 9 (SUC9) sucrose transporter and when the programmable nucleic acid modification system is a dCAS9-TV, an expression construct for expressing dCas9-TV and an expression construct for expressing gRNA that targets dCas9-TV to the nucleic acid sequence in the promoter region of a polynucleotide encoding SUC9 sucrose transporter can be incorporated into a plasmid vector. The plasmid vector comprising the expression construct for expressing dCas9-TV and the expression construct for expressing the gRNAs can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 104 or SEQ ID NO: 105. In some aspects, the plasmid vector comprising the expression construct for expressing dCas9-TV and the expression construct for expressing the gRNAs can comprise a nucleic acid sequence comprising at least about 75% or more, at least about 85% or more, at least about 95% or more, or 100% sequence identity with a nucleic acid sequence of SEQ ID NO: 104 or SEQ ID NO: 105.

In some aspects, when a programmable nucleic acid modification system targets a nucleic acid sequence in a promoter region of a polynucleotide encoding ANT and when the programmable nucleic acid modification system is a dCAS9-TV, an expression construct for expressing dCas9-TV and an expression construct for expressing gRNA that targets dCas9-TV to the nucleic acid sequence in the promoter region of ANT can be incorporated into a plasmid vector. The plasmid vector comprising the expression construct for expressing dCas9-TV and the expression construct for expressing the gRNAs can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 106 or SEQ ID NO: 107. In some aspects, the plasmid vector comprising the expression construct for expressing dCas9-TV and the expression construct for expressing the gRNAs can comprise a nucleic acid sequence comprising at least about 75% or more, at least about 85% or more, at least about 95% or more, or 100% sequence identity with a nucleic acid sequence of SEQ ID NO: 106 or SEQ ID NO: 107.

In some aspects, when a programmable nucleic acid modification system targets a nucleic acid sequence in a promoter region of a polynucleotide encoding BPEpa and BPEpb and when the programmable nucleic acid modification system is a dCAS9-SRDX, an expression construct for expressing dCas9-SRDX and an expression construct for expressing gRNA that targets dCas9-SRDX to the nucleic acid sequence in the promoter region of BPEpa and BPEpb can be incorporated into a plasmid vector. The plasmid vector comprising the expression construct for expressing dCas9-SRDX and the expression construct for expressing the gRNAs can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 108. In some aspects, the plasmid vector comprising the expression construct for expressing dCas9-SRDX and the expression construct for expressing the gRNAs can comprise a nucleic acid sequence comprising at least about 75% or more, at least about 85% or more, at least about 95% or more, or 100% sequence identity with a nucleic acid sequence of SEQ ID NO: 108.

### (a) Reversible Sterility Constructs

One aspect of the instant disclosure encompasses one or more expression constructs for expressing a reversible sterility/fertility restorer system in a legume plant (Fabaceae), part thereof, or plant cell. The one or more expression constructs comprise a sterility expression construct comprising a promoter operably linked to a sterility polynucleotide encoding a sterility factor and a fertility restorer expression construct comprising a promoter operably linked to a fertility restorer polynucleotide encoding a fertility restorer. reversible sterility/fertility restorer systems, sterility factors, and fertility restorers can be as described in Section I herein above.

As explained in Section I herein above, the genetically modified reversible sterility plants can be male-sterile or female sterile, wherein male-sterile plants can comprise a sterility expression construct comprising a male organ- or tissue-specific promoter operably linked to a sterility factor and female-sterile plants can comprise a sterility expression construct comprising a female organ- or tissue-specific promoter operably linked to a sterility factor. In some aspects, the fertility restorer construct expresses the fertility restorer in at least those specific cells of a sterile plant in which the sterility expression construct is expressed. In some aspects, the sterility construct and the fertility restorer construct comprise the same promoter. In some aspects, the male organ- or tissue-specific promoter is a pTA29a version of the pTA29 promoter. Importantly, the inventors unexpectedly discovered that the pTA29b version of the pTA29 promoter cannot be used in a sterility/fertility restorer system of the instant disclosure.

In some aspects, the pTA29a promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 38. In some aspects, the pTA29a promoter comprises a nucleic acid sequence comprising at least about 75% or more, at least about 85% or more, at least about 95% or more, or 100% sequence identity with a nucleic acid sequence of SEQ ID NO: 38.

In some aspects, the pTA29b promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 39. In some aspects, the pTA29a promoter comprises a nucleic acid sequence comprising at least about 75% or more, at least about 85% or more, at least about 95% or more, or 100% sequence identity with a nucleic acid sequence of SEQ ID NO: 39.

In some aspects, the sterility factor is barnase protein and the fertility restorer is barstar. When the sterility function is barstar, the sterility expression construct can comprise a tapetum-specific promoter operably linked to a polynucleotide encoding barnase which, when directed by the tapetum-specific promoter, is produced or overproduced in a stamen cell of the plant, kills or disables the stamen cell; wherein the fertility restorer expression construct comprises a tapetum-specific promoter operably linked to a polynucleotide encoding barstar; wherein crossing the sterile genetically modified plant to the fertility restorer genetically modified plant produces progeny comprising the sterility expression construct and the fertility restorer expression construct; wherein said progeny are fertile due to neutralization, blocking, prevention, or inhibition of the activity of barnase by barstar. In some aspects, the tapetum-specific promoter comprises a pTA29a promoter.

In some aspects, the sterility expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 43. In some aspects, the sterility expression construct comprises a nucleic acid sequence comprising at least about 75% or more, at least about 85% or more, at least about 95% or more, or 100% sequence identity with a nucleic acid sequence of SEQ ID NO: 43.

In some aspects, the fertility restorer expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 43. In some aspects, the fertility restorer expression construct comprises a nucleic acid sequence comprising at least about 75% or more, at least about 85% or more, at least about 95% or more, or 100% sequence identity with a nucleic acid sequence of SEQ ID NO: 43.

### (b) Plant Pollinator Phenotype

Another aspect of the instant disclosure encompasses one or more expression constructs for introducing a genetic modification into a legume plant (Fabacea), wherein the genetic modification modifies expression of a polynucleotide encoding a plant-pollinator floral phenotype. Modified expression of the polynucleotide encoding a plant-pollinator floral phenotype increases attraction of the pollinator to the plant, thereby resulting in an increased rate of cross-pollination. In some aspects, the genetic modification comprises a polynucleotide encoding a programmable transcription regulation system comprising a programmable targeting protein modified for transcriptional regulation of a locus. In some aspects, the programmable transcription regulation system comprises a programmable targeting protein modified for transcriptional regulation of a locus.

In some aspects, the polynucleotide encoding the programmable transcription regulation system comprises an expression construct for expressing dCas9-TV in flower cells and one or more expression constructs for expressing the gRNA in flower cells. In some aspects, the expression construct for expressing dCas9-TV in flower cells comprises a flower cell-specific promoter operably linked to a polynucleotide encoding dCas9-TV. In some aspects, the expression construct comprises an AP3 promoter operably linked to a polynucleotide encoding dCas9-TV. In some aspects, the expression construct for expressing the gRNA in flower cells comprises a constitutive promoter operably linked to a polynucleotide encoding the gRNA. In some aspects, the expression construct comprises an pU10 promoter operably linked to a polynucleotide encoding the gRNA.

In some aspects, the AP3 promoter comprising a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 36. In some aspects, the AP3 promoter comprises a nucleic acid sequence comprising at least about 75% or more, at least about 85% or more, at least about 95% or more, or 100% sequence identity with a nucleic acid sequence of SEQ ID NO: 36.

In some aspects, the polynucleotide encoding dCas9-TV comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 66. In some aspects, the polynucleotide encoding dCas9-TV comprises at least about 75% or more, at least about 85% or more, at least about 95% or more, or 100% sequence identity with a nucleic acid sequence of SEQ ID NO: 66.

In some aspects, the expression construct comprises an AP3 promoter operably linked to a polynucleotide encoding dCas9-TV comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 62. In some aspects, the expression construct comprises an AP3 promoter operably linked to a polynucleotide encoding dCas9-TV comprises at least about 75% or more, at least about 85% or more, at least about 95% or more, or 100% sequence identity with a nucleic acid sequence of SEQ ID NO: 62.

In some aspects, the pU10 promoter comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 30. In some aspects, the pU10 promoter comprises a nucleic acid sequence comprising at least about 75% or more, at least about 85% or more, at least about 95% or more, or 100% sequence identity with a nucleic acid sequence of SEQ ID NO: 30.

In some aspects, a gRNA comprises a sequence which binds to a polynucleotide sequence in a promoter region of the polynucleotide encoding ANT comprises a nucleic acid sequence of SEQ ID NO: 1, SEQ ID NO: 2, SEQ ID NO: 3; or any combination thereof. In some aspects, a gRNA comprising a sequence which binds to a polynucleotide sequence in a promoter region of the polynucleotide encoding ANT comprises a nucleic acid sequence of SEQ ID NO: 5, SEQ ID NO: 6, or SEQ ID NO: 7, or any combination thereof. In some aspects, a gRNA comprising a sequence which binds to a polynucleotide sequence in a promoter region of the polynucleotide encoding ANT comprises a nucleic acid sequence of SEQ ID NO: 1, SEQ ID NO: 2, SEQ ID NO: 3, SEQ ID NO: 5, SEQ ID NO: 6, and SEQ ID NO: 7, or any combination thereof.

In some aspects, the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4. In some aspects, the target is a nucleic acid sequence in a polynucleotide comprising at least about 75% or more, at least about 85% or more, at least about 95% or more, or 100% sequence identity with a nucleic acid sequence of SEQ ID NO: 4.

In some aspects, the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 8. In some aspects, the target is a nucleic acid sequence in a polynucleotide comprising at least about 75% or more, at least about 85% or more, at least about 95% or more, or 100% sequence identity with a nucleic acid sequence of SEQ ID NO: 8.

In some aspects, the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4 or SEQ ID NO: 8. In some aspects, the target is a nucleic acid sequence in a polynucleotide comprising at least about 75% or more, at least about 85% or more, at least about 95% or more, or 100% sequence identity with a nucleic acid sequence of SEQ ID NO: 4 or SEQ ID NO: 8.

In some aspects, the gRNA comprises a nucleic acid sequence of SEQ ID NO: 1, SEQ ID NO: 2, SEQ ID NO: 3; or any combination thereof, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4. In some aspects, the gRNA comprises a nucleic acid sequence of SEQ ID NO: 1, SEQ ID NO: 2, SEQ ID NO: 3; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 4.

In some aspects, the gRNA comprises a nucleic acid sequence of SEQ ID NO: 5, SEQ ID NO: 6, or SEQ ID NO: 7; or any combination thereof, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 8. In some aspects, the gRNA comprises a nucleic acid sequence of SEQ ID NO: 5, SEQ ID NO: 6, or SEQ ID NO: 7; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 8.

In some aspects, the gRNA comprises a nucleic acid sequence of SEQ ID NO: 1, SEQ ID NO: 2, SEQ ID NO: 3, SEQ ID NO: 5, SEQ ID NO: 6, and SEQ ID NO: 7; or any combination thereof, wherein the target is a nucleic acid sequence in a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4 or SEQ ID NO: 8. In some aspects, the gRNA comprises a nucleic acid sequence of SEQ ID NO: 1, SEQ ID NO: 2, SEQ ID NO: 3, SEQ ID NO: 5, SEQ ID NO: 6, and SEQ ID NO: 7; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 4 or SEQ ID NO: 8.

In some aspects, a polynucleotide encoding the gRNA comprises an expression construct comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 65. In some aspects, an expression construct comprising a polynucleotide encoding the gRNA comprises at least about 75% or more, at least about 85% or more, at least about 95% or more, or 100% sequence identity with a nucleic acid sequence of SEQ ID NO: 65.

### VI. Methods

A further aspect of the present disclosure encompasses a method of generating a reversibly sterile genetically modified legume plant (Fabaceae) cell, legume plant, part thereof, or legume plant seed thereof. The method comprises generating a genetically modified plant comprising a sterility expression construct comprising a promoter operably linked to a sterility polynucleotide encoding a sterility factor. Expression of the sterility factor in the plant causes the plant to be sterile and wherein expression of the fertility restorer in the plant of (a) neutralizes, blocks, offsets, prevents, or inhibits the sterility factor causing the plant to be fertile. In some aspects, a method of the instant disclosure further comprises generating a genetically modified plant comprising a fertility restorer expression construct comprising a promoter operably linked to a fertility restorer polynucleotide encoding a fertility restorer. Crossing a plant comprising a sterility expression construct to a plant comprising a fertility restorer expression construct produces fertile progeny plants. Genetically modified plants generated using methods of the instant disclosure can be as described in Section I herein above.

The method comprises introducing a sterility expression construct comprising a promoter operably linked to a sterility polynucleotide encoding a sterility factor into a legume plant or plant cell. The plant or plant cell is then grown under conditions whereby the nucleic acid expression construct expresses the sterility factor. In some aspects, the method can further comprise introducing a fertility restorer expression construct comprising a promoter operably linked to a fertility restorer polynucleotide encoding a fertility restorer into a legume plant or plant cell. Nucleic acid constructs expressing a sterility factor and a fertility restorer can be as described in Section III herein above.

The expression constructs can comprise an exogenous nucleic acid molecule such as a chimeric nucleic acid of the disclosure. The term “exogenous” as used herein refers to a nucleic acid molecule originating from outside the plant cell. An exogenous nucleic acid molecule can be, for example, the coding sequence of a sterility factor or a fertility restorer. An exogenous nucleic acid molecule can have a naturally occurring or non-naturally occurring nucleotide sequence and can be a heterologous nucleic acid molecule derived from a different organism or a different plant species than the plant cell into which the nucleic acid molecule is introduced or can be a nucleic acid molecule derived from the same plant species as the plant cell into which it is introduced. The exogenous nucleic acid can or cannot be integrated in the plant cell's genome. When said exogenous nucleic acid/gene is not integrated, transient expression of the nucleic acid/gene occurs in the plant cell.

Non-limiting examples of methods of introducing genetic modifications in a plant cell can be transposon insertion mutagenesis, T-DNA insertion mutagenesis, T-DNA activation tagging, chemically or radio-induced mutagenesis site-directed mutagenesis, directed evolution, homologous recombination, introducing and expressing in a plant a nucleic acid encoding a sterility factor or a fertility restorer.

A further aspect of the present disclosure encompasses a method of generating a genetically modified legume plant (Fabaceae), part thereof, or plant cell thereof, the genetically modified plant comprising an improved a plant-pollinator floral phenotype. The method comprises generating a genetically modified plant comprising a genetic modification of at least one target nucleic acid sequence in a polynucleotide encoding a floral trait. The genetic modification can be an expression construct encoding a floral trait, a nucleic acid modification in the polynucleotide encoding a floral trait, or any combination thereof. Genetically modified plants generated using methods of the instant disclosure can be as described in Section I herein above.

The method comprises introducing one or more nucleic acid expression constructs for expressing a floral trait or one or more nucleic acid expression constructs for expressing an engineered nucleic acid modification system into a legume plant or plant cell. The engineered nucleic acid modification system can be a programmable transcription regulation system. The plant or plant cell is then grown under conditions whereby the nucleic acid expression construct expresses floral trait or the programmable nucleic acid modification system. Expressing the programmable nucleic acid modification system introduces a nucleic acid modification in the polynucleotide encoding floral trait, thereby generating a genetically modified plant comprising an improved plant-pollinator floral phenotype. The genetically modified plant can be as described in Section II herein above. The expression constructs for expressing the floral phenotype and the engineered nucleic acid modification system for introducing the nucleic acid modification can be as described in Section V herein below.

The expression constructs modify the expression of a polynucleotide encoding the floral trait. The plant or plant cell is then grown under conditions whereby the polynucleotide encoding the floral trait or the engineered nucleic acid modification system in the plant or plant cell. Expressing the floral phenotype of the engineered nucleic acid modification system modifies the expression of the polynucleotide encoding the floral phenotype in the plant.

### (a) Legume Plants and Pollinators

A legume is a plant in the family Fabaceae (or Leguminosae), or the fruit or seed of such a plant. When used as a dry grain, the seed is also called a pulse. Farmed legumes can belong to many agricultural classes, including forage, grain, blooms, pharmaceutical/industrial, soil-enhancing fallow/green manure, and timber species. Well-known legumes include beans, locust bean, fenugreek, soybean (Glycine), chickpeas, peanuts, garden beans, cowpea, mungbean, lentils, lupins, mesquite, carob, guar, tamarind, alfalfa, lima bean, fava bean, lentils, chickpea, crown vetch (Vicia sp.), hairy vetch, stylo (stylosanthes), adzuki bean, Leucaena, Albizia, trifolium, common bean (Phaseolus sp.), field bean (Pisum sp.), clover (Melilotus sp.), Lotus, trefoil, lens, and false indigo. Legumes are notable in that most of them have symbiotic nitrogen-fixing bacteria in structures called root nodules. For that reason, they play a key role in crop rotation.

Legumes can be self-pollinated or cross-pollinated. Many legumes such as peas, lima beans, snap beans, subterranean clover, garden beans, soybeans, peas and peanuts likewise largely self-pollinate. Often, pollen is shed onto the stigma while the flower is still closed, early in the morning before bees are present. So, bees aren't absolutely necessary, but the flowers are still fertile and occasionally use the pollen brought by bees. Non-limiting examples of closely self-pollinated include Macroptilium atropurpureum ‘Siratro’, Macroptilium lathyroides, Centrosema pubescens, Neonotonia wightii, and Lotononis bainesii. In some aspects, a legume plant of the instant disclosure is soybean.

In some aspects, the plant is a soybean (Glycine sp.). Non-limiting examples of Glycine sp. include Glycine hispida, Glycine max, and Glycine soja. In another aspect, the plant is Glycine hispida. In some aspects, the soybean plant is a domesticated soybean plant. In one aspect, the plant is Glycine max.

Pollinators can be any animal that intentionally or accidentally pollinate a flower, including insects, arachnids, bats, and other mammals. Commonly seen pollinators include honeybees, alkali bees, leafcutter bees, and bumblebees. Although honeybees are usually abundant, they are rather inefficient as pollinators when compared with alkali bees. Their large numbers tend to offset this deficiency. Among the Alkali bees, Nomia melanderia is very common. Leafcutter bees (Megachile spp.) are usually present but in low numbers. Bumblebees (Bombus spp.) occur locally in moderate abundance. In some aspects, a pollinator of the instant disclosure is a bumblebee. In other aspects, a pollinator of the instant disclosure is a honeybee.

### (b) Producing Hybrid Seed

Yet another aspect of the present disclosure encompasses a method of producing hybrid seed of a legume plant. The method comprises planting seeds of a sterile genetically modified plant comprising a reversible sterility phenotype and seeds of a genetically modified fertility restorer plant and allowing the seeds to germinate and grow into plants. The method further comprises allowing the fertility restorer plants to pollinate the sterile plants to thereby produce the hybrid seed on the sterile plant. The sterile genetically modified plant and the genetically modified fertility restorer plant can be as described in Sections I and III herein above.

An additional aspect of the instant disclosure encompasses a method of producing hybrid seed of a legume plant. The method comprises planting seeds of genetically modified plants comprising an improved plant-pollinator floral phenotype and allowing the plants to pollinate to thereby produce the hybrid seed.

Another aspect of the instant disclosure encompasses a method of synergistically producing hybrid seed of a legume plant. The method comprises planting seeds of a sterile genetically modified plant comprising an improved plant-pollinator floral phenotype and seeds of a fertility restorer genetically modified plant comprising an improved plant-pollinator floral phenotype. The method further comprises allowing the seeds to germinate and grow into plants and allowing the fertility restorer plants to pollinate the sterile plants to thereby produce the hybrid seed on the sterile plant. The combination of the improved plant-pollinator floral phenotype and the reversible sterility phenotype create a synergistic cross pollination phenotype

(c) Introduction into the Cell

The method comprises introducing a nucleic acid construct expressing one or more proteins or nucleic acid sequences into a cell of interest. As explained above, the one or more proteins or nucleic acid sequences can be encoded on one or more than one nucleic acid sequence. The one or more nucleic acid constructs can be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, viral vectors, magnetofection, lipofection, impalefection, optical transfection, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

### (d) Culturing a Cell

The method further comprises culturing a cell under conditions suitable for expressing the engineered protein. Methods of culturing cells are known in the art. In some aspects, the cell is from an animal, fungi, oomycete or prokaryote. In some aspects, the cell is a plant cell, plant, or plant part. When the cell is in tissue ex vivo, or in vivo within a plant or within a plant part, the plant part and/or plant can also be maintained under appropriate conditions for insertion of the donor polynucleotide. In general, the plant, plant part, or plant cell is maintained under conditions appropriate for cell growth and/or maintenance. Those of skill in the art appreciate that methods for culturing plant cells are known in the art and can and will vary depending on the cell type. Routine optimization can be used, in all cases, to determine the best techniques for a particular cell type. See for example, in Santiago et al. (2008) PNAS 105:5809-5814; Moehle et al. (2007) PNAS 104:3055-3060; Urnov et al. (2005) Nature 435:646-651; Lombardo et al. (2007) Nat. Biotechnology 25:1298-1306; and Taylor et al. (2012) Tropical Plant Biology 5:127-139.

### VII. Kits

A further aspect of the present disclosure provides kits for generating a genetically modified legume plant (Fabaceae) cell, legume plant, part thereof, or legume plant seed thereof comprising a reversible sterility phenotype, a genetically modified legume plant comprising an improved plant-pollinator phenotype, a genetically modified plant comprising a reversible sterility phenotype and an improved plant-pollinator phenotype, or for producing hybrid seed of a legume plant.

The kit can comprise one or more expression constructs for expressing a reversible sterility system, one or more expression constructs for introducing an improved plant-pollinator phenotype into a legume plant, or any combination thereof. The kit can also comprise one or more genetically modified legume plants comprising a reversible sterility phenotype, one or more genetically modified legume plants comprising an improved plant-pollinator phenotype, or one or more genetically modified plant comprising a reversible sterility phenotype and an improved plant-pollinator phenotype.

The genetically modified plants can be as described in Sections I, II, and Ill herein above. The constructs can be as described in Sections II and V herein above.

The kits can further comprise transfection reagents, cell growth media, selection media, in vitro transcription reagents, nucleic acid purification reagents, protein purification reagents, buffers, and the like. The kits provided herein generally include instructions for carrying out the methods detailed below. Instructions included in the kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

A “genetically modified” plant refers to a plant in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, the term “engineered” when applied to a targeting protein refers to targeting proteins modified to specifically recognize and bind to a nucleic acid sequence at or near a target nucleic acid locus. A “genetically modified” plant refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

The term “nucleic acid modification” refers to processes by which a specific nucleic acid sequence in a polynucleotide is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the calcium channel activity; and glycosylation and/or other modifications of the translation product, if required for proper expression and function. The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

The term “allele” as used herein refers to one of two or more different nucleotide sequences that occur at a specific locus.

“Backcrossing” refers to the process whereby hybrid progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular marker Data, pp. 41-43. The initial cross gives rise to the F1 generation: the term “BC1” then refers to the second use of the recurrent parent; “BC2” refers to the third use of the recurrent parent, and so on.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, an “elite line” is any line that has resulted from breeding and selection for superior agronomic performance.

A “favorable allele” is the allele at a particular locus that confers, or contributes to, a desirable phenotype, e.g., increased GS tolerance, or alternatively, is an allele that allows the identification of plants with decreased GS tolerance that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

“Genome” refers to the total DNA, or the entire set of genes, carried by a chromosome or chromosome set.

The terms “phenotype”, or “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple led, or, more generally, the term genotype can be used to refer to an individual's genetic make-up for all the genes in its genome.

The term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA that is too common to be due merely to new mutation. A polymorphism must have a frequency of at least 1% in a population. A polymorphism can be a single nucleotide polymorphism, or SNP, or an insertion/deletion polymorphism, also referred to herein as an “indel”.

The term “progeny” refers to the offspring generated from a cross.

A “progeny plant” is generated from a cross between two plants.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences may also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs may be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Introduction of Barnase Barstar in Soybean Produces a Rescuable Male Sterility System for Hybrid Breeding

In this study, barnase/barstar male-sterility/male-rescue technology were integrated into soybean to test the potential for this biotechnological approach to enable large-scale hybrid breeding in soybean. barnase expressing lines were generated that consistently exhibited complete male-sterility, and two different rescue lines were generated that expressed barstar at equal or higher levels than barnase. It was shown that a higher dose of barstar relative to barnase is essential for producing a successful rescue of male-fertility in the hybrid generation of barnase male-sterility lines. This present study opened up new resources for investigating heterosis and advancing the future of breeding in soybean.

### Construct Design and Synthesis

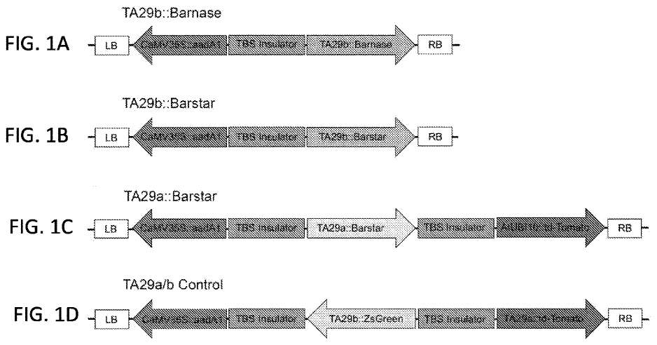

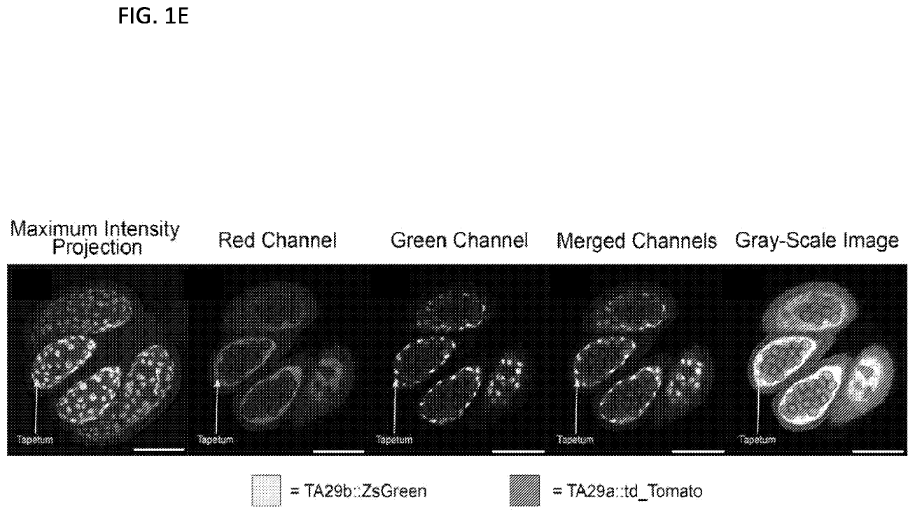

Constructs for tapetum-specific expression of barnase/barstar proteins were designed based on sequences from Bacillus amyloliquefaciens. Two soybean paralogs for TA29 were identified using a BLAST search with the Arabidopsis promoter for TA29 (At5g07230) as a query sequence. This query uncovered TA29a (Glyma.09G144000) and TA29b (Glyma. 16G197100). Expression data for the two soybean TA29 paralogs was downloaded from the JGI Plant Gene Atlas to ensure full rescue of barnase with the barstar rescue lines, a relatively weaker expressing TA29b::barnase construct (SEQ ID NO: 42), and two versions of barstar constructs, TA29a::barstar, and TA29b::barstar were generated (FIG. 1A-1C, and SEQ ID NO: 43 and SEQ ID NO: 44). A control construct was also generated (SEQ ID NO: 58). In addition, to confirm that TA29a/TA29b are expressed in tapetal cells, a dual reporter construct expressing TA29a::td-Tomato and TA29b::ZsGreen in opposite directions with an insulator (Transformation booster sequence (TBS) from petunia) between the two reporters was generated (FIG. 1D). Cloning was achieved by the Wisconsin Crop Innovation Center (WCIC) using the Golden Gate MoClo Plant Tool Kit and final vector backbone pAGM4673 provided in the kit.

### Plant Transformation

Transgenic soybean lines were generated at the WCIC using a proprietary protocol that involves Agrobacterium tumefaciens-mediated transformation of cultured shoot meristems. Positive transformants were selected based on kanamycin resistance and confirmed with transgene-specific primers. At least 8 independent transformants were generated for each construct.

### Genotyping Transformants

All T1 plants used in this study were genotyped for transgene inheritance using primers targeted to the aadA1 antibiotic resistance gene, which was present in all of the constructs, and construct-specific targets: barnase, barstar, ZsGreen, and td-Tomato (Primers, PCR conditions, and construct details are in Table 1). Genotyping PCRs were performed using Promega Hotstart Green MasterMix following the recommended conditions from the manufacturer (Promega, Madison, WI, U.S.A.).

TABLE 1
Primers, PCR conditions and construct details

| Primer Name | Primer Sequence | Annealing Temperature |
|---|---|---|
| aadA1_Forward | CTCCATCAACAGCAGATCCA | 63° C. |
| SEQ ID NO: 79 | | |
| aadA1 Reverse | CGCACAATCCCACTATCCTT | 63° C. |
| SEQ ID NO: 80 | | |
| barstar Forward | GAAAGAACTCGCTCTGCCAG | 63° C. |
| SEQ ID NO: 81 | | |
| barstar_Reverse | TGCAAAACACTTTCAGCTCCA | 63° C. |
| SEQ ID NO: 82 | | |
| barnase_Forward | TTCGACGGTGTTGCAGACTA | 63° C. |
| SEQ ID NO: 83 | | |
| barnase_Reverse | CGGTTACTAAAGATGTCGCCC | 63° C. |
| SEQ ID NO: 84 | | |
| ZsGreen_Forward | CCAGTTATCGGTCATCTTCTTC | 60° C. |
| SEQ ID NO: 85 | | |
| ZsGreen Reverse | TACCACGAGTCCAAGTTCTAC | 60° C. |
| SEQ ID NO: 86 | | |
| td-Tomato Forward | TGGCTACTACTACGTCGATAC | 60° C. |
| SEQ ID NO: 87 | | |
| td-Tomato_Reverse | GTGACGTCCTTCTGATCTTTC | 60° C. |
| SEQ ID NO: 88 | | |

### Growth Chamber and Greenhouse Conditions

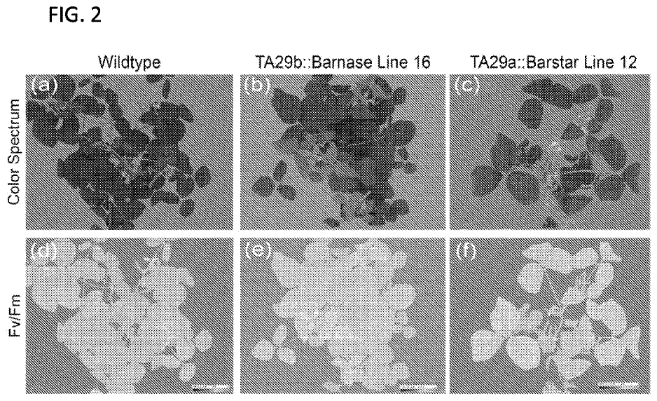

Plants were grown in greenhouse (daytime temp: 23.9° C. in heating and 26.7° C. in cooling; nighttime temp: 20° C. in heating and 22.8° C. in cooling, light for 14 hours) and growth chamber (23° C., 100% light for 16 hours, R/H at 60%) facilities at Cornell University. Plants in FIG. 2, panels (a)-(f) were grown in greenhouse conditions at the Donald Danforth Plant Science Center (daytime temp: 25° C. day/23° C. night; 35% minimum humidity; light for 14 hours (supplemental light when sunlight is below 400 W/m2 and shade curtain pulls down to 50% when sunlight is over 900 W/m2 and pulls down to 100% when sunlight is over 1000 W/m²).

Soybean Crosses with TA29b::barnase Lines

Mature TA29b::barnase flowers were identified based on the emergence of petals from under the sepal whorl. At this stage in normal flower development, the pollen is viable and the stigma is receptive to pollination. To uncover the stigma, sepals and petals were manually removed from TA29b::barnase flowers and exposed anthers from pollen donors were rubbed directly onto the stigma surface. To increase the probability of fertilization, multiple crosses were made from the same pollen donor onto each stigma.

### Staining and Imaging Samples

To obtain light microscopy images of whole flowers and individual whorls, soybean flowers were dissected under a Leica M205 FCA fluorescent stereo microscope and imaged with a DMC6200 camera.

For propidium iodide staining, samples were fixed in fresh FAA (50% Ethanol, 10%, 37% Formaldehyde, 5% glacial acetic acid) in small glass vials. Samples were vacuum infiltrated until the majority of the samples sunk to the bottom of the vial. The flower samples were transferred to cuvettes and placed in closed cups containing fresh FAA and incubated overnight at 4° C. The samples were transferred to 50% ethanol at room temperature, and then gradually dehydrated through an ethanol series for 20 minutes each, followed by an incubation in 2×100% ethanol for an hour each, and gradually rehydrated (20 minutes each) to 100% water. Rehydrated samples were rinsed with sterile, autoclaved water twice, and then stained with Propidium Iodide (2 mL of 1 mg/ml PI stock in 100 mL+0.02% DMSO) for one hour. Samples were rinsed 2×20 minutes in water, gradually dehydrated to 100% ethanol, and then transferred into 50:50 EtOH: Methyl Salicylate for 2 hours, followed by 100% methyl salicylate for 1-2 weeks at 4° C. for clearing. Completely cleared samples were determined based on their translucent appearance and imaged on an LSM880 Confocal multiphoton inverted-1880 Zeiss microscope. Samples were imaged using either 514 nm or 561 nm excitation, collecting in the 566-718 nm emission range, with laser power ranging from 0.02-0.024%.

To image TA29a::td-Tomato/TA29b::ZsGreen dual reporter lines, flowers were cleared following a previously published ClearSee protocol to reduce background autofluorescence. Young soybean flower buds (approximately 3-4 mm in length) were fixed with 4% (w/v) paraformaldehyde for 2 hours in PBS under a vacuum at room temperature. The fixed tissue was washed twice with PBS for 1 minute each and cleared with ClearSee solution (10% Xylitol (w/v), 15% Sodium deoxycholate (w/v), 25% Urea (w/v)) at room temperature for 4 weeks under gentle shaking (120 rpm). The clearing solution was changed every other day. After confirming that the tissue was translucent, young buds were imaged using an LSM880 Confocal multiphoton inverted—i880 Zeiss microscope. ZsGreen fluorescence was imaged with a 488 nm laser, collecting in the 493-556 nm emission range, with laser power at 0.025%. td-Tomato fluorescence was imaged with a 561 nm laser, collecting in the 566-691 nm emission range, with laser power at 0.02%. The FIJI software was used to merge image data from td-Tomato and ZsGreen.

### Pollen Viability and Pollen Tube Growth Assays

Pollen viability for barstar, WT, and barnase/barstar rescue crosses was quantified using a previously published protocol. Briefly, anthers were dissected during anthesis and placed in a 15-ml conical tube. Pollen was released from the anthers by vortexing. The released pollen was resuspended in pollen viability solution [PVS, 290 mM sucrose, 1.27 mM Ca (NO3) 2, 0.16 mM boric acid, 1 mM KNO3) containing 0.001% (wt/vol) fluorescein diacetate, and 10 μM propidium iodide (stock solutions: 1% (wt/vol) fluorescein diacetate in acetone; 2 mM PI in water]. The pollen was stained for 15 min at 28° C. and then centrifuged. The PVS containing FDA and PI was replaced with PVS alone and the stained pollen was placed on a microscope slide, and then imaged by confocal microscopy on a Zeiss 880 LSCM microscope. The FDA was excited with a 488 nm laser and its signal collected at 493-584 nm. The PI was excited with a 561 nm laser and its signal collected at 584-718 nm. Pollen was hand-counted within the FIJI software using the cell counter plugin.

To highlight pollen tube development, a modified protocol for using aniline blue staining was followed. Hand-crossed and self-pollinated flower buds were harvested and fixed in Farmer's Solution (3:1 100% ethanol: glacial acetic acid) 24 hrs post-pollination. Buds were incubated at room temperature for 5 minutes, and then transferred into 5 M NaOH. Samples were incubated at 65° C. for 30 minutes to soften the plant tissue, the buds were carefully rinsed three times in distilled water, and then placed in decolorized aniline blue solution [0.1% (w/v) aniline blue dissolved in 0.1 M K3PO4 and decolorized by incubating overnight at 37° C. in the dark]. After 30 minutes, the carpel was dissected, the trichomes were removed, and the sample was arranged in a drop of aniline blue solution on the microscope slide. The cover slip was added, and pressure was gently applied to squash the tissue. Slides were sealed with clear topcoat nail polish to avoid drying, and examined with an LSM880 Confocal multiphoton inverted-1880 Zeiss microscope, exciting with a 405 nm diode laser and collecting in the 415-735 nm emission range, or on an Olympus upright Metamorph microscope using UV fluorescence and a DAPI filter.

### CropReporter™ Imaging

Plants were imaged over the course of multiple days at late vegetative and flowering stages using the CropReporter™ imaging box (Netherlands Plant Eco-phenotyping Centre (NPEC), Wageningen University & Research and Utrecht University). From a top view, the CropReporter™ collected dFv/Fm, FO, Fm, RGB, NIR, CHL, and dCHL measurements in dark and light adapted plants to calculate the Anthocyanin-index (Anthocyanin: (R550)-1-(R700)-1), the Chlorophyll-index (Chlorophyll: (R700)-1-(RNIR)-1), and the Fv/FM (Fv/Fm=Fm-F0/Fm). Fv/Fm was used to determine if a stressor was affecting photosystem II efficiency. The Phenovation Data Analysis software v548 was used to separate the plant from its background and segregate collected data into individual heatmaps for each type of information collected. The heatmaps visualized regions of high anthocyanin pigmentation, high photosystem II efficiency, and overall plant stress.

### Molecular Characterization

For genotyping, DNA was extracted using a modified protocol. 400 μl of Edward's Buffer [40% (v/v) 5 M NaCl and 60% (v/v) extraction buffer (200 mM Tris/HCL pH 7.5, 250 mM NaCl, 25 mM EDTA, 0.5% SDS)] was added to a 1.5 ml microcentrifuge tube containing a young trifoliate leaf, ground up with a micropestle and incubated at 60° C. for 30 minutes. After incubation, samples were centrifuged at 9,000×g for 5 minutes. 270 ul of the supernatant was transferred to a new tube and gently mixed with 270 ul of isopropanol. The new tubes were incubated at −20° C. for 15 minutes, followed by centrifugation at full speed for 5 minutes. Following centrifugation, the supernatant was removed, and the pellet was washed with 70% EtOH, centrifuged at full speed for 5 minutes, and decanted. Residual EtOH at the bottom of the tube was removed via micropipette and the tube was left open to dry at 60° C. for 10 minutes. Once all the ethanol was removed, sterile water was added and the tube was incubated at 60° C. for a final 30 minutes.

### Quantifying Barnase/Barstar Expression

Gene expression was quantified using qRT-PCR. RNA was extracted from young soybean buds using TRIzol (Ambion, Austin, TX, U.S.A), following the manufacturer's instructions for plant extraction. GlycoBlue™ Blue Coprecipitant (Invitrogen, Waltham, MA, U.S.A) was added to each sample during the RNA precipitation step to facilitate recovery of the RNA pellet. The concentration and quality of the RNA was checked using a DeNovix DS-11 FX+Spectrophotometer/Fluorometer. RNA samples with 260/280 ratios ranging from 1.7-2.2 were converted to cDNA using QuantiTect™ Reverse Transcriptase (Qiagen, Hilden, Germany). cDNA concentrations were quantified using a nanodrop and diluted to 1-10 ng of cDNA, and 1 ul of diluted cDNA was added to Power Up™ SYBR Green Master Mix (Applied Biosystems, Waltham, MA, U.S.A) along with gene-specific primers targeting ZsGreen, td-Tomato, barnase, barstar, and a housekeeping gene, UKN1 (Table 2). The qRT-PCR housekeeping gene, UKN1 was selected based on a literature review of stable targets used for soybean gene expression experiments. Samples were run on a Bio-Rad CFX96 Optics Module with a C1000 Touch Base set at 50° C. for 2 minutes followed by 95° C. for 2 minutes for predenaturation. For 39 cycles, the machine was set at 95° C. for 10 seconds and 60° C. for 30 seconds to denature, anneal, and elongate. Downstream data analysis was performed with an excel spreadsheet to compute the delta Ct value (normalized to housekeeping gene, UKN1) and Student's t-test for significance.

TABLE 2
Primer Name, Primer Sequence, Anneal Temperature and Target Gene

| qRT-PCR Primer | | Annealing | |
|---|---|---|---|
| Name | Primer Sequence | Temp | Target Gene |
| barnase_qRT- | GGGTAGCTTCAAAGGGTAAC | 60° C. | N/A |
| PCR_Forward | | | |
| SEQ ID NO: 89 | | | |
| barnase_qRT- | TCCAGGCAATTTACCTTCAC | 60° C. | N/A |
| PCR_Reverse | | | |
| SEQ ID NO: 90 | | | |
| barstar_qRT- | TGGAGAACAGATCAGGAGTATC | 60° C. | N/A |
| PCR_Forward | | | |
| SEQ ID NO: 91 | | | |
| barstar_qRT- | CAGTCCCAAAGAGCATCAAG | 60° C. | N/A |
| PCR_Reverse | | | |
| SEQ ID NO: 92 | | | |
| ZsGreen_qRT- | CCAGTTATCGGTCATCTTCTTC | 60° C. | N/A |
| PCR_Forward | | | |
| SEQ ID NO: 93 | | | |
| ZsGreen_qRT- | TACCACGAGTCCAAGTTCTAC | 60° C. | N/A |
| PCR_Reverse | | | |
| SEQ ID NO: 94 | | | |
| td-Tomato_qRT- | TGGCTACTACTACGTCGATAC | 60° C. | N/A |
| PCR_Forward | | | |
| SEQ ID NO: 95 | | | |
| td-Tomato_qRT- | GTGACGTCCTTCTGATCTTTC | 60° C. | N/A |
| PCR_Reverse | | | |
| SEQ ID NO: 96 | | | |
| UKN1_qRT- | CTGGATCTGGCACTCTTATTC | 60° C. | Glyma.12g02310.1 |
| PCR_Forward | | | |
| SEQ ID NO: 97 | | | |
| UKN1_qRT- | GAGCTGAGTTTGCATGTTTG | 60° C. | Glyma.12g02310.1 |
| PCR Reverse | | | |
| SEQ ID NO: 98 | | | |

### Designing Tapetum-Specific Promoters to Develop a Rescuable Male-Sterility System in Soybean

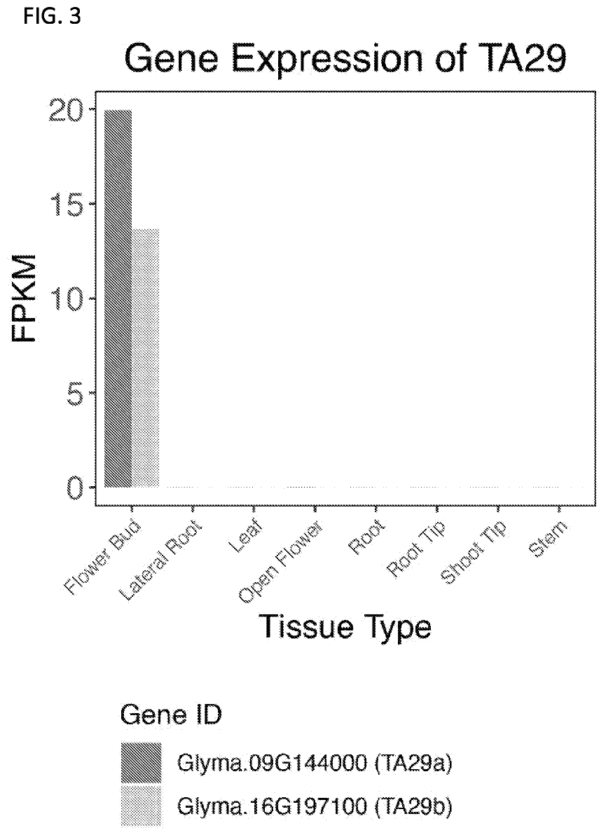

To isolate the cytotoxic ribonuclease activity of barnase and rescue activity of barstar to the tapetal cell layer of soybean anthers, two soybean paralogs were identified for the previously characterized tapetum-specific Arabidopsis gene TA29 (Atg07230). TA29a (Glyma.09G144000) and TA29b (Glyma. 16G197100) are specifically expressed in unopened soybean flowers with TA29a exhibiting higher expression than TA29b (FIG. 3). To confirm that TA29a/TA29b are specifically expressed in tapetal cells, a dual reporter line was generated with TA29a::tdTomato and TA29b::ZsGreen (FIG. 1D). In this reporter line, tapetum-specific expression of both fluorophores was observed at early stages of microspore development (FIG. 1D), indicating that these TA29 paralogs are appropriate for directing barnase and barstar expression in soybean.

Since barnase is a potent ribonuclease, it was decided to take advantage of the differential expression of TA29a/TA29b to create a rescue system in which barstar dosage is consistently higher than barnase. It was hypothesized that this differential dosage would limit the presence of uninhibited barnase that could negatively impact male-fertility in the F1 rescues. To test this hypothesis, one TA29b::barnase expressing construct, and two TA29a::barstar and TA29b: barstar rescue constructs were designed. In addition, TA29a::barstar with a AtUBI10::td-Tomato fluorophore was tagged to facilitate quick identification of successful crosses onto TA29b::barnase pre-genotyping (FIG. 1A-1C).

### Tapetum-Specific Expression of Barnase Produces Complete Male-Sterility

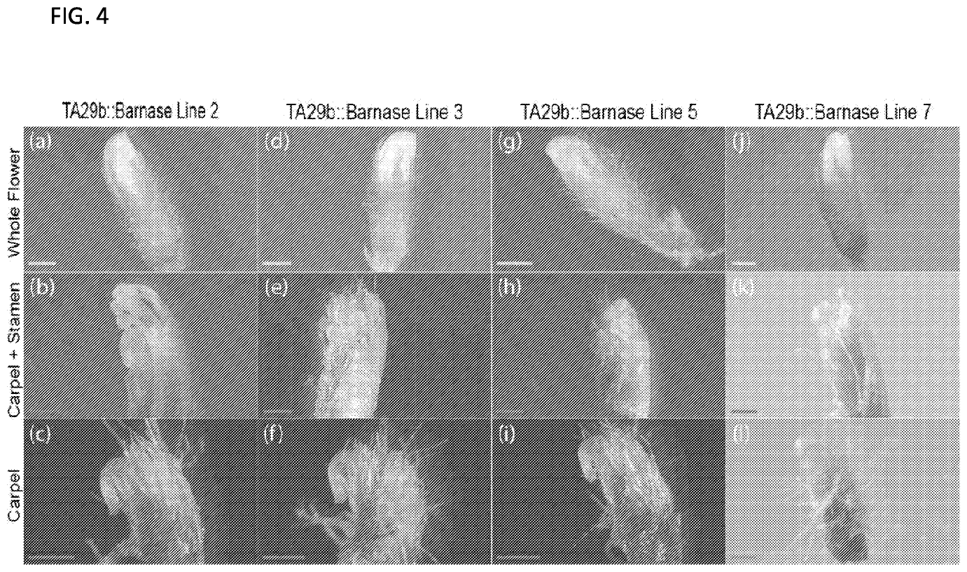

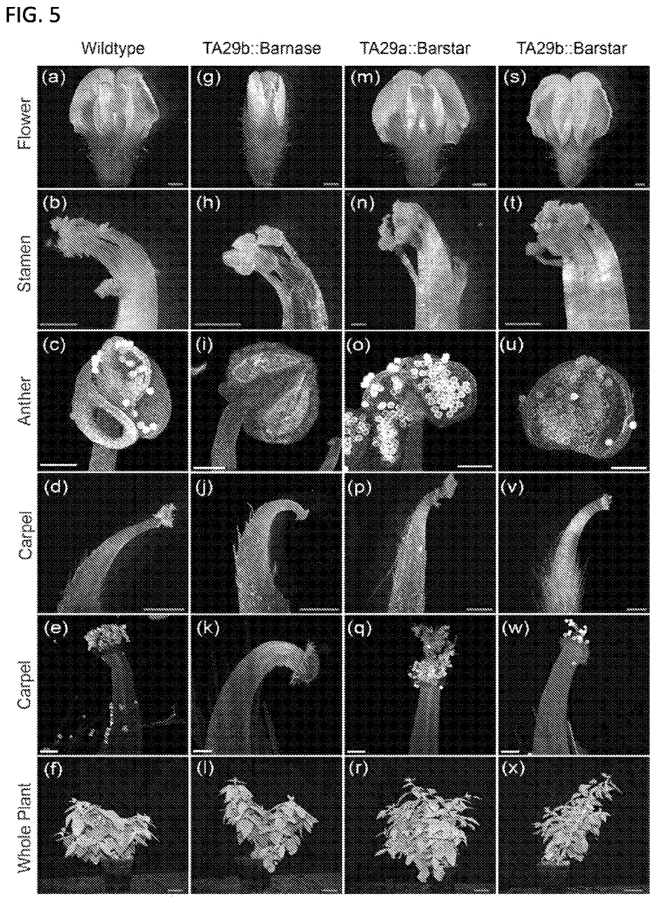

Of the 13 independent TA29b::barnase lines that we generated, 12 exhibited consistent male-sterility phenotypes (FIG. 4). Wildtype soybean plants produce both cleistogamous (closed) and chasmogamous (open) flowers at maturity. Interestingly, while there were no other alterations to overall flower morphology and size, it was noticed that all 12 of the male-sterile barnase lines only produced cleistogamous flowers (FIG. 5, Panel (g)). To evaluate the effect of barnase expression on stamen and carpel formation, flowers at peak reproductive maturity were dissected and imaged (FIG. 5, Panels (g)-(k)). All of the TA29b::barnase male-sterile lines formed anthers that were misshapen, either opaque white/light yellow or semi-translucent in appearance, and failed to form pollen grains (FIG. 5, Panels (h)-(k), FIG. 4, Panels (b), (e), (h), (k)). In contrast, wildtype anthers were golden and shedding pollen at maturity (FIG. 5, Panels (b) and (c)). propidium iodide (PI) staining and confocal imaging were used to further investigate the impact of barnase on internal anther organization and identify the cellular basis for male-sterility in these lines. In WT post-meiotic anthers, it was observed pollen grain release from mature pollen sacs, whereas, in barnase-expressing plants, the cavity of the anther lacked microspores and instead exhibited degenerating cellular debris that fluoresced brightly with PI staining (FIG. 5, Panels (c) and (i)).

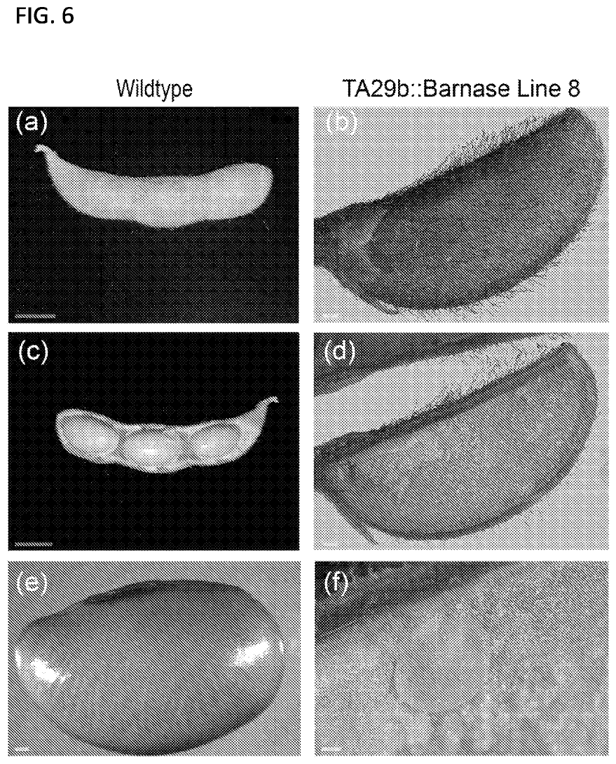

To investigate whether TA29b::barnase impacted carpel formation, overall carpel shape and papillar cell morphology were imaged using a dissecting microscope and confocal imaging, respectively. When compared to WT, it was noticed that mature carpels in barnase expressing plants tended to curve inward, positioning the stigma towards the center of the flower where the free stamen is located (FIG. 5, Panels (d), (e), (j), (k)). It was found this phenotype to be consistent across independent TA29b::barnase transformants (FIG. 4, Panels (c), (f), (i), and (l)). WT carpels also exhibited slight curvature, however, the stigma tended to face upward in these flowers. Congruent with our findings that the barnase plants were completely male-sterile, zero fertilized pods were found on any of these lines (FIG. 6). Interestingly, in lieu of fertilized pods, we did observe parthenocarpic pod formation on the TA29b::barnase (FIG. 6). These stubby pods were formed as a result of ovule development in the absence of fertilization, and could easily be distinguished from viable pods based on their length, presence of unfertilized ovules in place of seeds, and greatly reduced size (FIG. 6).

Since barstar is an inhibitor of barnase, It was not expected that TA29a::barstar and TA29b::barstar to differ in phenotype from WT. In line with this hypothesis, the petals, carpels, and stamens for these two transgenic lines were indistinguishable from those formed on WT flowers (FIG. 5, Panels (a)-(e), (m)-(q), (s)-(w)). Furthermore, the two barstar lines produced numerous fertile pods similar to the WT controls. To test whether barnase and barstar constructs impacted the overall phenotype of WT, we collected a spectrum of image-based measurements that relate to vegetative health and photosynthetic capacity using a CropReporter™ system (Netherlands Plant Ecophenotyping Center) and corresponding Phenovation Data Analysis software v548. We did not identify any noticeable change in the vegetative health of either the barnase or barstar transgenic lines relative to WT plants (FIG. 2). This data demonstrates that the barstar constructs have no measurable impact on plant phenotype, and the barnase construct primarily impacts male reproductive development.

### Rescuing Male-Fertility to Generate F1 Hybrids

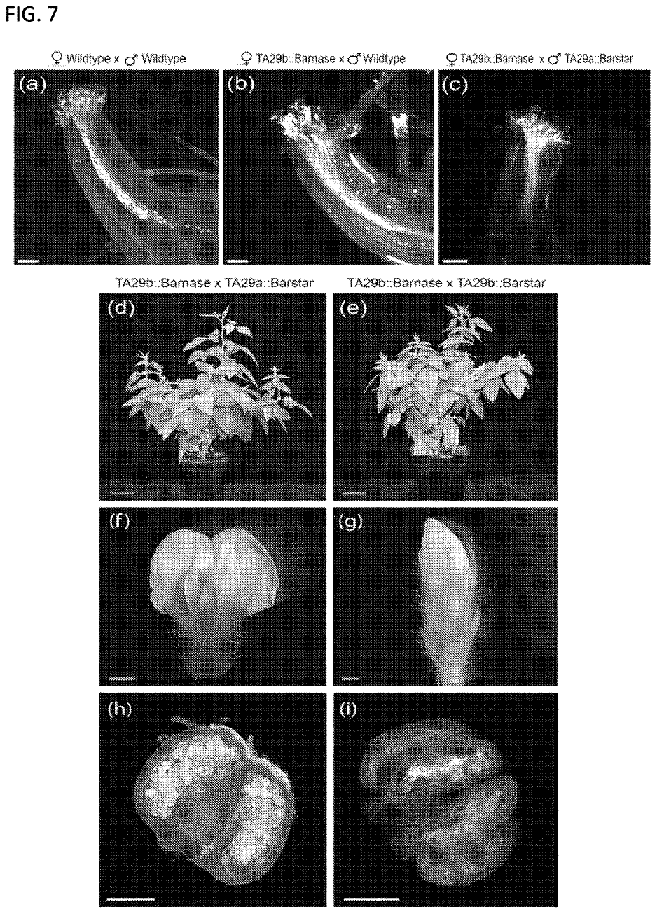

To test whether barnase-expressing carpels are receptive to pollen, WT and TA29a::barstar pollen were crossed onto TA29b::barnase stigmas, and used aniline blue staining to track pollen tube elongation. Relative to the WT control, it was observed unaltered pollen grain germination and growth on the TA29b: barnase carpels (FIG. 7, Panels (a)-(c)). The confocal images show that pollen grains were able to adhere to the surface of the stigma, and regardless of the pollen and/or carpel genotypes, we observed comparable levels of pollen tube growth at 24 hours post-pollination (FIG. 7, Panels (a)-(c)). This data demonstrates that TA29b::barnase carpels are reproductively viable.

As mentioned above, two barstar rescue lines were constructed to test whether a higher dosage of barstar relative to barnase is sufficient to fully rescue male-fertility in F1 hybrids. three different TA29a::barstar and two different TA29b::barstar lines were crossed onto TA29b::barnase plants, genotyped F1 seedlings for the presence of barnase and barstar transgenes (Table 1), and examined male-fertility in these F1 hybrids. In line with our findings for the parental transgenic lines (TA29b::barnase, TA29a::barstar, and TA29b::barstar), the presence of both transgenes had no effect on overall vegetative growth and morphology (FIG. 7, Panels (d) and (e).

At reproductive maturity, it was noticed that, like the TA29b::barnase parental lines, all of the TA29b::barnase×TA29b::barstar F1 flowers were cleistogamous (i.e. closed at maturity), while TA29b::barnase×TA29a::barstar F1 plants developed both cleistogamous and chasmogamous flowers, similar to WT plants (FIG. 7, Panels (f), (g)). To test whether pollen formation was rescued by crossing with either barstar construct, PI-stained anthers were examined from our F1 plants using confocal microscopy. While the anther sacs from the TA29b::barnase×TA29a::barstar crosses were full of pollen, similar to WT anthers, it was found that TA29b::barnase×TA29b::barstar anthers formed zero pollen grains (FIG. 7h,7i). Instead, flowers from TA29b::barnase×TA29b::barstar F1s exhibited brightly stained cellular debris inside of the anther sacs, similar to the phenotype that we characterized for TA29b::barnase flowers (FIG. 7, Panel (i) and FIG. 5, Panel (i)). Moreover, the only pods that were formed on the TA29b::barnase×TA29b::barstar crosses were parthenocarpic, indicating that the plants were completely male-sterile. Therefore, the crosses with TA29b::barstar was referred to as failed and those with TA29a::barstar as successful rescues.

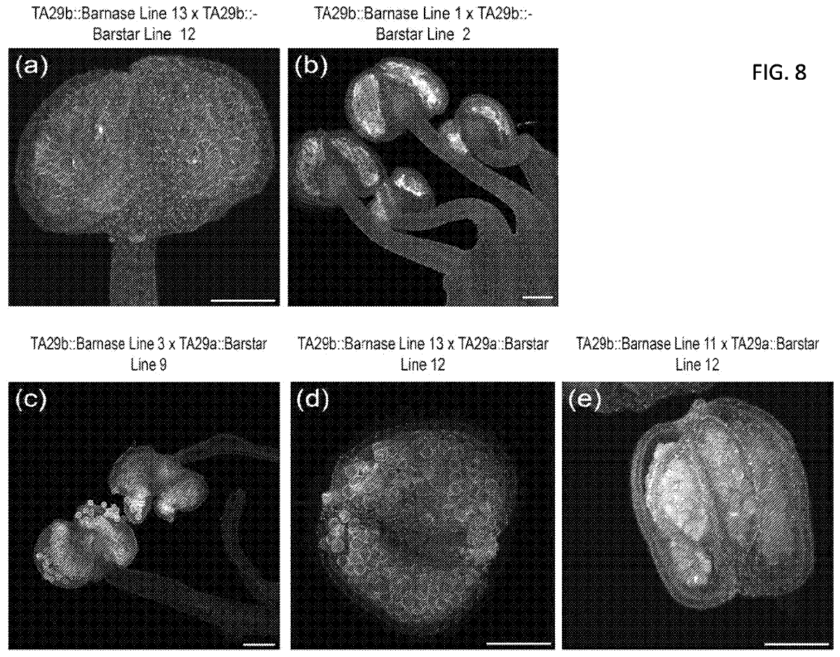

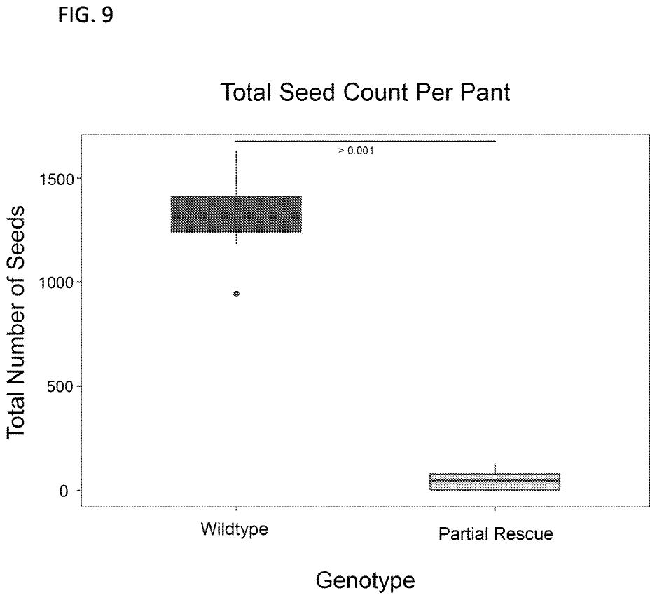

Not all TA29b::barnase×TA29a::barstar crosses rescued pollen formation to the same degree. It was observed one barnase/barstar combination, TA29b::barnase-line 11×TA29a::barstar-line 12, that produced clumpy pollen grains that failed to efficiently release from the anthers (FIG. 8, Panel (e)). This partial rescue exhibited both cleistogamous and chasmogamous flowers (similar to the full rescue plants); however, aggravated shaking was required to dehisce and release clumpy pollen from the anthers for imaging (FIG. 8, Panel (e)). These partial-rescue plants set a mix of reproductive and parthenocarpic pods, however, they formed significantly fewer seeds overall than WT plants (FIG. 9). These results indicate that further tuning is needed across the independent barnase/barstar lines to ensure consistent rescue phenotypes.

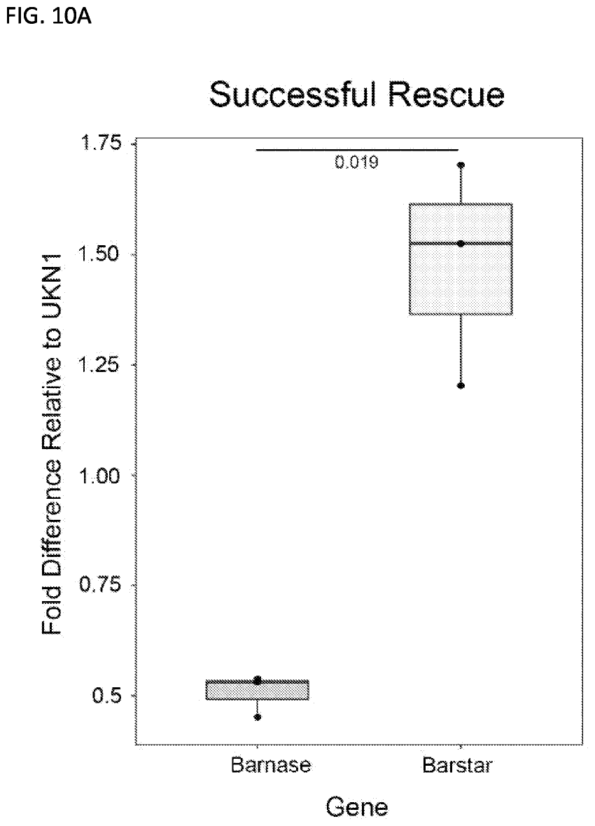

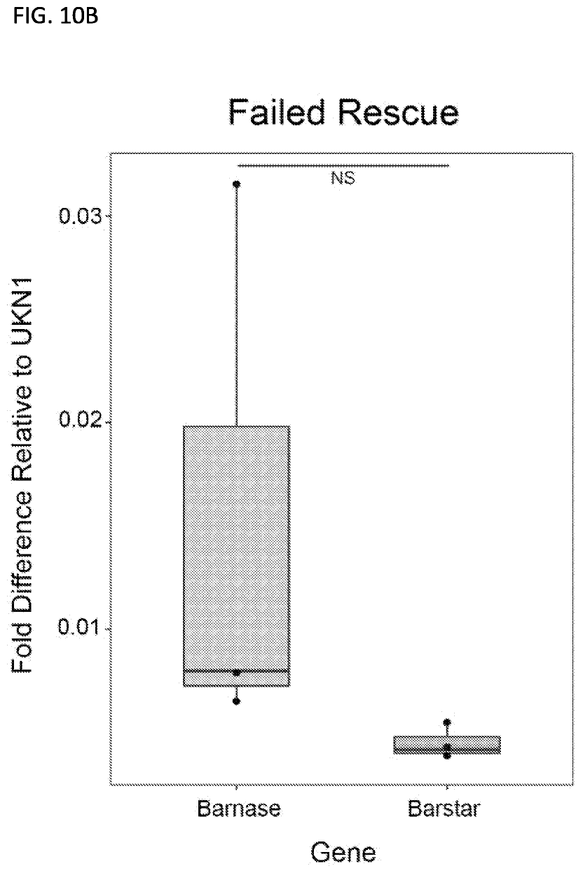

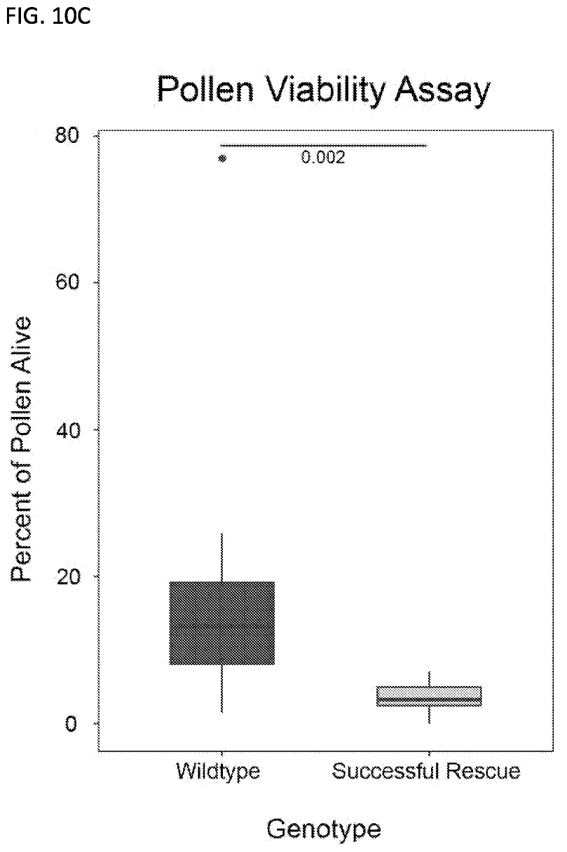

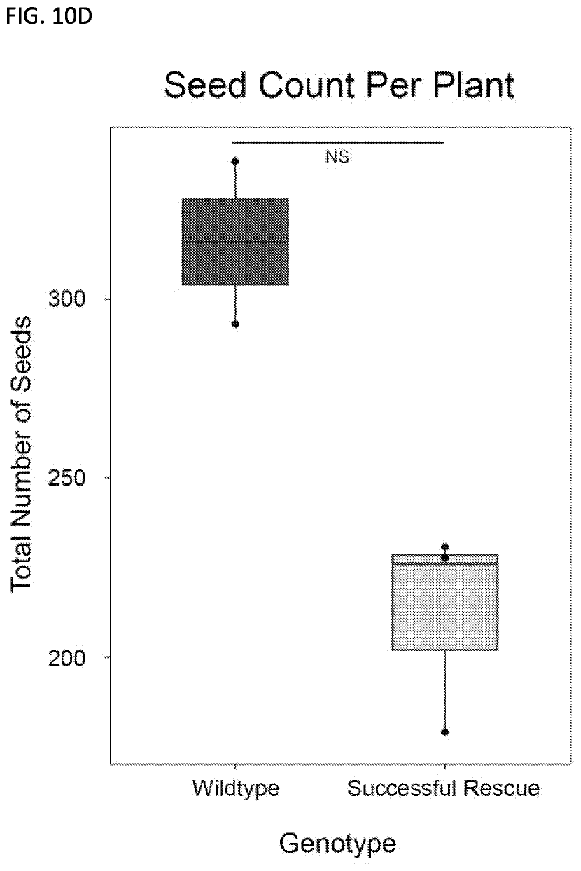

To examine whether successful rescue of pollen formation in TA29b::barnase×TA29a::barstar plants can be attributed to higher barstar expression relative to barnase, qRT-PCR was performed for barnase and barstar on flowers from failed versus successful rescues. It was found that F1 plants with rescued fertility exhibited significantly higher barstar expression relative to barnase, while failed rescue flowers showed no significant differences between barnase and barstar expression (FIG. 10, Panels (a), (b)). This data supports a model in which higher expression of barstar relative to barnase is necessary to rescue pollen formation in F1 hybrids.

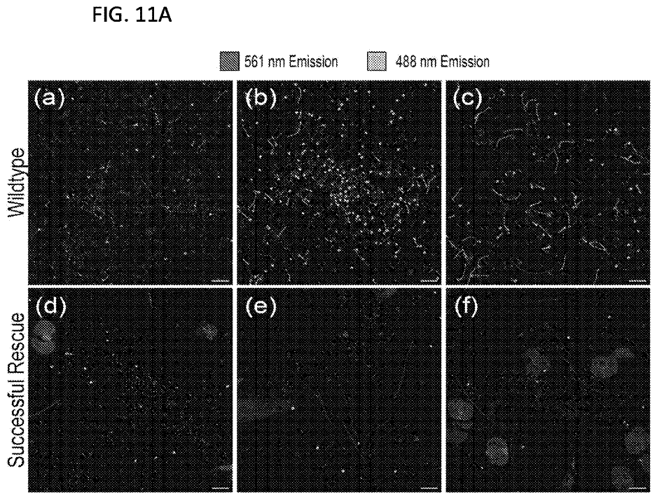

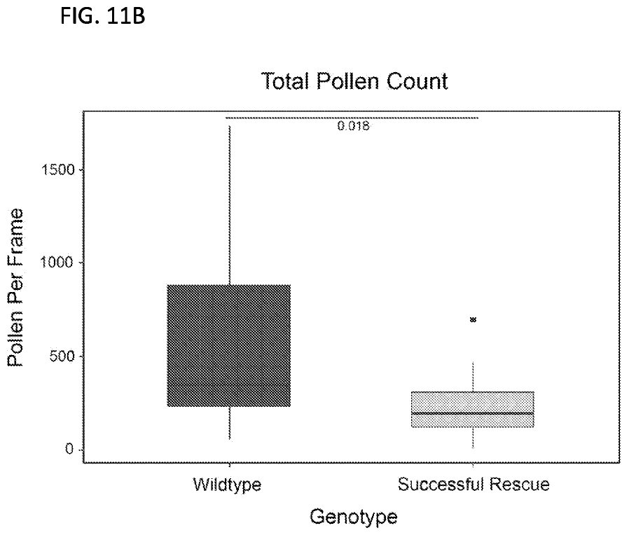

Next, it was tested whether the pollen formed in successful rescue flowers is alive, using a fluorescein diacetate (FDA)/PI pollen viability assay (FIG. 10, Panel (c), FIG. 11, Panels (a)-(f)). It was found that all of the successful rescue crosses produced viable pollen; however, overall viability counts for these lines were significantly lower (3.6% viability on average) than in WT flowers (15.7% on average). Moreover, it was noticed that WT anthers released substantially more pollen than the successful rescue anthers (FIG. 11, Panel (g)). Despite these differences in pollen counts and percent viability, we found that the rescue crosses produced sufficient levels of viable pollen to promote self-fertilization and the formation of reproductive seed pods (FIG. 10, Panels (c), (d)). When comparing WT plants to successful rescue plants (TA29b: barnase-line 13×TA29a::barstar-line 12) in growth chamber conditions, it was found no significant difference in total seed count. These results demonstrated that crossing with TA29a::barstar can restore reproductive fitness to previously sterile plants containing TA29b::barnase (FIG. 10, Panel (d)).

### Discussion

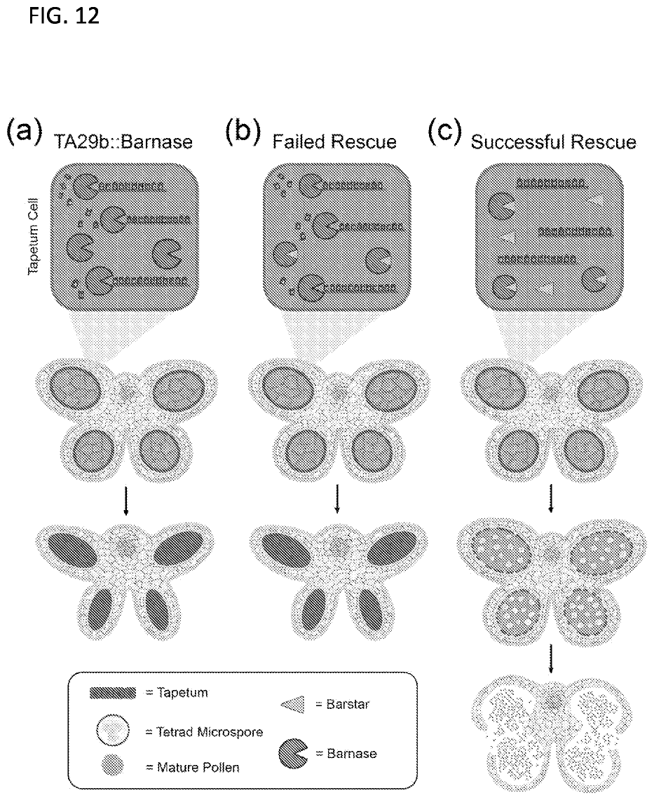

The present disclosure demonstrated that barnase and barstar can be effectively used to create an obligate outcrossing breeding system in soybean. Notably, it was shown that a higher dosage of barstar relative to barnase is required to produce successful male-rescue in the F1 generation (FIG. 12, Panels (a)-(c)). barnase is a potent ribonuclease; when expressed in the tapetum it destroys this cell layer and inhibits microspore maturation. The present disclosure showed that cytotoxic ablation of the tapetum using barnase is sufficient to produce complete male-sterility (FIG. 12, Panel (a)). It was also demonstrated that male-fertility can be rescued in a dosage-dependent manner by expressing the barnase-inhibitor, barstar. While equal dosage of barstar to barnase failed to rescue fertility (FIG. 12, Panel (b)), when barstar was expressed at significantly higher levels than barnase, it was able to recover self-fertile F1 hybrids (FIG. 12, Panel (c)).

Due to the potent cytotoxic nature of barnase, a small fraction of uninhibited ribonuclease is sufficient to induce male-sterility. In the present model, the dosage of barstar inhibitor must exceed that of the ribonuclease to ensure complete inactivation of barnase, restoring male-fertility (FIG. 12, Panel (c)). Even with this successful rescue, it was observed some TA29b::barnase×TA29a::barstar F1 hybrids that only exhibited partial rescue phenotypes with reduced fertility. These partial rescues indicated that further optimization of the relative dosage of barnase to barstar is needed in order to ensure full rescue and heightened seed set in the F1 hybrids. Tuning of barnase and barstar dosage could be achieved by modifying the relative strength of the TA29 promoters through the addition of cis regulatory elements, or identifying new tapetum specific promoters that can be used to regulate barnase/barstar expression. Relative to other major crops, reproductive development in soybean is insufficiently studied. Soybean currently lacks key enabling resources such as a detailed floral organ expression atlas, and cell type specific expression profiling. Such resources are essential for achieving a hybrid breeding system through optimized reproductive rewiring.

To fully realize the potential for hybrid breeding, programmed male-sterility/male-rescue is only part of the solution; it would also need to recruit insect vectors to facilitate outcrossing. Pollinator ecology studies clearly indicate that honeybees are the primary pollinators in soybean fields. These studies show that a mutually beneficial interaction exists between bees and soybean fields. Soybean fields provide a source of nectar in midwestern agricultural landscapes, and in the reverse direction, significant increases in crop yield are associated with the presence of nearby apiaries. The present disclosure strongly indicated that honeybees could serve as an ecologically beneficial choice for facilitating soybean outcrossing. Building a better understanding of the floral phenotypes that impact honeybee preferences in soybean is essential to effectively incorporate honeybees as vectors for hybrid breeding.

Furthermore, a clear framework was currently lacking for predicting strong heterotic combinations in soybean, which precludes our ability to project the overall benefits that could be derived from applying hybrid breeding to this major crop. This is in large part due to the technical challenges of generating hybrid seed for this species. Indeed, almost all hybrid soybean seed is generated by making crosses by hand, which means that research on heterosis in this crop has been restricted to a small number of crosses with low sample sizes. Though information is limited, existing studies show that particular combinations of inbred parents can lead to significant yield boosts in the F1 generation. However, to date, these studies are limited to narrow geographic regions due to the challenges of generating hybrid seed. Given that soybean is grown globally across a broad range of environments, current knowledge on hybrid vigor in soybean does not necessarily apply to this crop's expansive geographic range, emphasizing the need for efficient hybrid breeding. Obligate outcrossing with the barnase/barstar lines that we present in this paper provides a new resource that can be used to amplify hybrid seed sets, enabling large-scale trials for heterosis in this major crop.

Hybrid breeding in soybean has the potential to increase the productivity of one of the most planted and consumed crops in the Americas, yet it has remained largely unexplored. This is in part due to the limitations of current approaches, which have failed to produce reliable obligate outcrossing in soybean. The present disclosure provides a key enabling technology to produce obligate outcrossing in soybean.

### Example 2. Flower Morphology

Hybrid breeding for increased vigor is a major driver of increased crop yield. Male-sterile legume lines can significantly benefit from improved plant-pollinator floral phenotype for efficient pollen delivery through pollinator attraction. Although the potential for hybrid breeding to promote heterosis in soybean has not been exhaustively studied, small field trials demonstrate that heterotic combinations do exist in soybean, and thus serve as an untapped source for increased yield. In this study, the floral phenotype of soybean plants were modified and shown to improve recruitment of pollinators to serve as vectors for hybrid pollination. The plants provide a transformative strategy for hybrid breeding in soybean. This technological advance could have a tremendous impact on multiple sectors of agricultural crop production.

Pollinators are attracted to larger floral displays in the violet-blue range and prefer nectar with higher sucrose content. This study focuses on gene candidates for these phenotypes in soybean. The biochemical basis of purple versus white petal color has been genetically characterized directly in soybean, and while regulators for petal size and sucrose nectar content have yet to be characterized in soybean; gene candidates for these phenotypes have been identified in Arabidopsis. For example, larger petals can be achieved by increasing expression of AINTEGUMENTA (ANT) and decreasing expression of BIGPETALp (BPEp), and higher sucrose nectar content could potentially be achieved by increasing the expression of the sucrose transporter SUC9.

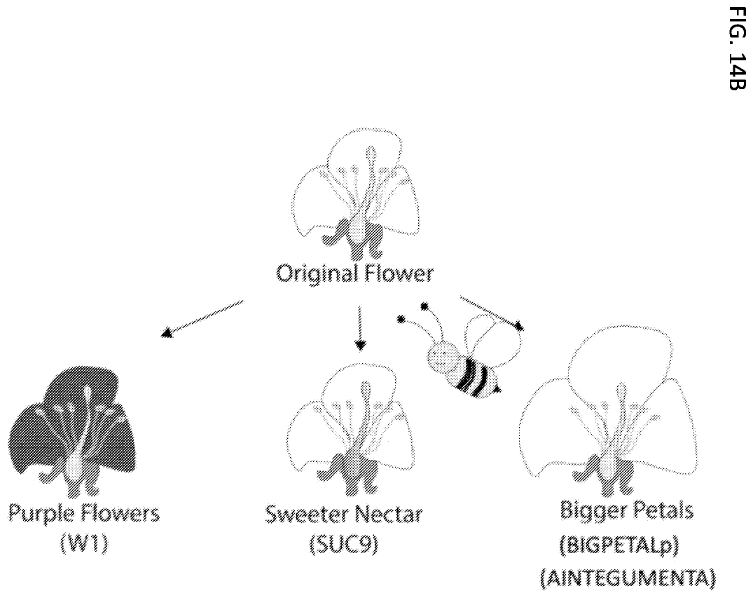

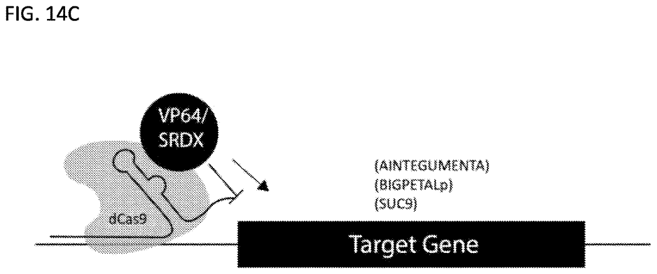

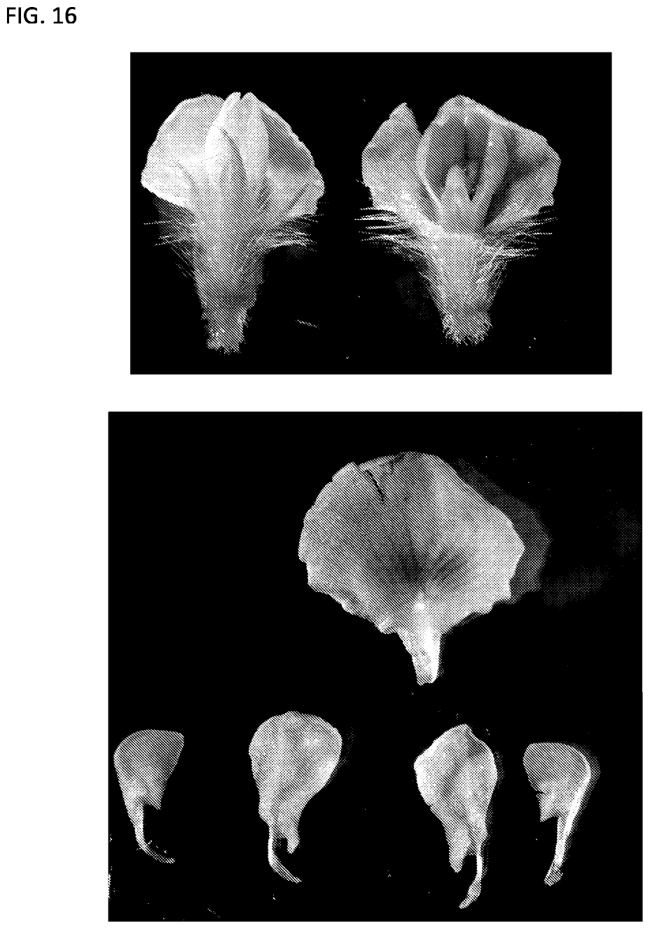

Target genes for transcriptional activation/repression (highlighted in red) were selected based on their expression in developing floral buds and sequence homology with Arabidopsis genes (FIG. 14A). Soybean is a diploidizing tetraploid, and thus contains two paralogous genes for every Arabidopsis candidate (e.g. ANTa and ANTb). A series of transgenic lines were generated to test whether modified W1, ANTa/ANTb, BPEpa/BPEpb, and SUC9a/SUC9b transgenic lines can produce enhanced, pollinator-attracting floral phenotypes in soybean as explained below. Specifically, A W1 complementation line that transfers the functional, purple-flowered W1 allele into a white-flowered background, and six lines that target ANTa/ANTb, BPEpa/BPEpb, and SUC9a/SUC9b paralogs for transcriptional activation (for ANT and SUC9) or repression (for BPEp) were generated using a deactivated Cas9 (dCas9) that recruits transcriptional activators or repressors (respectively) to targeted promoters (FIG. 14B). For modifying the expression of genes affecting petal size and sweet nectar phenotypes guide RNA were used to direct deactivated Cas9 “dCas9” carrying either an activator (VP64) or a repressor (SRDX) to the promoters of target genes to modify gene expression (FIG. 14C). Purple petals were obtained by expressing a purple petal allele of the W1 gene under the control of the white petal W1 allele of the gene (FIG. 16).

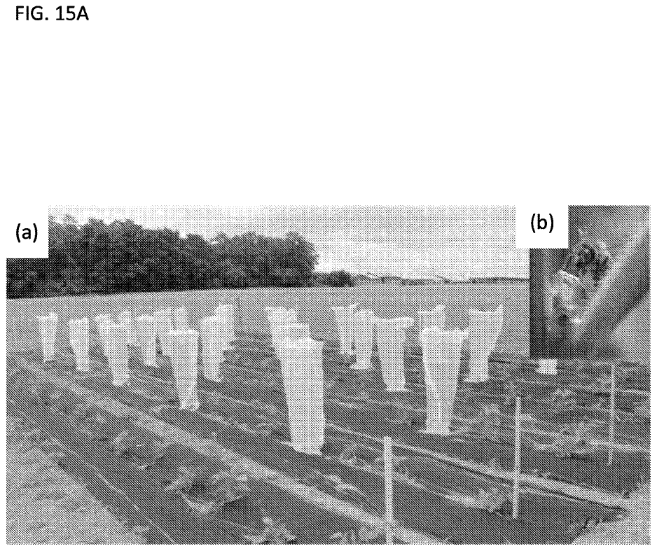

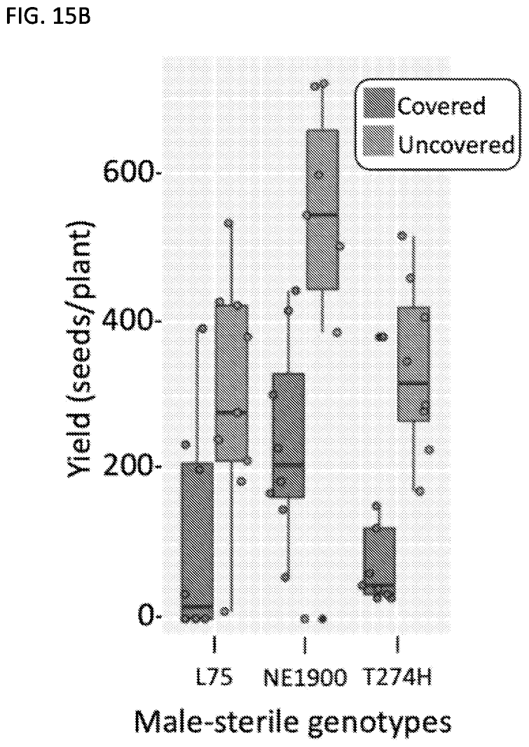

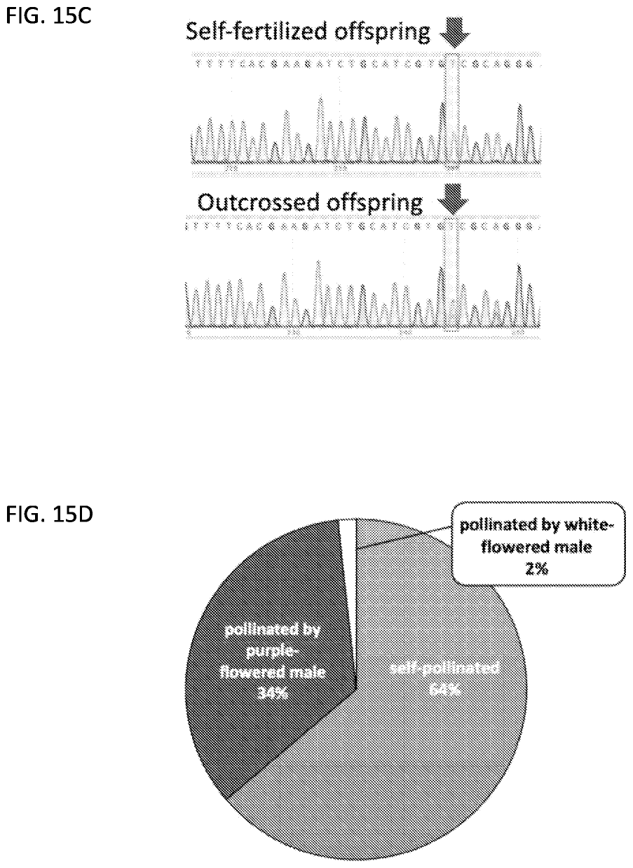

Field studies (FIG. 15A, Panel (a)) using genetically modified plants with improved flower phenotypes generated in this study in combination with male-sterile plants showed that bee pollinators (FIG. 15A Panel (b)) visit flowers of the genetically modified plants more frequently that flowers of wild type plants (FIG. 15B). Genotype data for offspring collected from male-sterile lines provides hard evidence for outcrossing with surrounding male-fertile lines (FIG. 15C). Genotyping for 100 offspring from male sterile mothers, shows that the majority of offspring still form from self-fertilization (64%), with an additional 34% of offspring coming from a purple-flowered pollen donor and 2% from a white-flowered pollen donor (FIG. 15D). It is noted that the male-sterile genotypes used in this study were male-sterile soybean lines available before the inventors developed the male sterile lines described in the instant disclosure and Example 1. For that reason, a high level of self-fertilization was observed as the male sterility in these genotypes is not 100% effective.

### Example 3. Integration of Barnase/Barstar System with the Floral Phenotype Modifications

In this study, the barnase/barstar system developed as described in Example 1 is integrated with the most attractive floral phenotype modifications generated as described in Example 2. Results show a significant improvement of cross-pollination when compared to results obtained using male-sterile soybean lines available before the inventors developed the barnase/barstar male sterile lines described in the instant disclosure and Example 1.

TABLE 3
Sequence listing

| SEQ. ID | | |
|---|---|---|
| NO. | Sequence | Notes |
| 1 | AAAAAATGTGAATGTGGATGAGG | gRNA 3 of |
| | | ANTa |
| 2 | AAATTTATAACATTTACATCTGG | gRNA 2 of |
| | | ANTa |
| 3 | TGATGTCTTTAGACCAGACAAGG | gRNA 1 of |
| | | ANTa |
| 4 | ATATTATAAAAAAAATATTGTTAATAATTAAAGATTAAGATAGATATAAT | Promoter |
| | TTAATATAATTGCAAATTAGATACTATTTTATGTCTATACTTACAAGGAG | Region |
| | ATGAAAACAGATTAACACTTGAATTAATTAATGTGATGTTTCAATTTAAT | Sequence |
| | TAAATGTCTCAAATTTAAATTTCGACTCCTTAACTACATTAAATAGTTAA | + 5′ UTR |
| | GAAAACTTTTGTAGAAGATCTTACTCAATCAATACTTGTGATGTCTTTAG | of ANTa |
| | ACCAGACAAGGAGATAAAAAAAAAAGAAAAATAAGAGATTGATATTTTGA | |
| | TAACGTAACATATATAAAAAAAAGAGAGAAATCTAAAGTGTTGATAAATT | |
| | GTTTGTAATTAATAAGCATCCAAAAATTATTATTATATTTTAAAAGTCAA | |
| | TTAATATGTGTATATGTATATTTCAATTTGGAAATAAAAGTGATATAATT | |
| | TAGTTTATTGATCCAGATGTAAATGTTATAAATTTTTGATATCTTATTAA | |
| | ATTTATATATTTTAAAAAAATGTGAATGTGGATGAGGTAAAAGTATAAAA | |
| | TATAATAACCCAAAAATATATATAAAATATTAAATAAACTTTTTTGGCTG | |
| | ATTTCTGAATCTTTTCCCAAATCCCCTCACCGTCTATATCTTGGTCATTT | |
| | GCGCTCACTAGATTCACCTACTAGCATTCAAGTTTTCAGAAACTGACCAA | |
| | AAAAAGTTCATCTCACCATTACCATTTTGGGCTCTTTCTTCTTATCCACT | |
| | TTGAAAAATTAGATTCTGGGTTTCACATAGGGTTTAAGAAAAAGCTAAGT | |
| | CTAAAGAGGGGTGGCAGAGTTTTCTCTCTTTTTTTTTTTATAAAAAAATA | |
| | AGAGAAAAAAAAAAAGCAAAAGACTTTGTGTGTATATATACACATAAATA | |
| | CACAGATCCCTATGGAATGAAGTGGGTGCTCCCAATATCACTTGCTAGTA | |
| | ACACTTATTCCTTCTGCTTAGTTTAAAGAGACCAGTTTTATTATGCTCGA | |
| | GTGAAGAAAGAGAGAGAGAAAAGAGAGAGTTTGGTTTGCTTGTTTTGTTG | |
| | GTGTGAGAAAGAAAAACCTTGCTGTAGCTAAACAACAAAAACCAAGTCTT | |
| | CATTGGTAACAAGAAGATTATTATTTTTATATGATTTGTTTATTTATCAC | |
| | CCAATGATTGACTTTGCCTAGCTGCTGCTGCTACGAGAGAAGATACTGCT | |
| | GGTGGTGGTGCTAGCAATAGCAAGTTTAAAGTTCAAACCTTTTTCAAGTA | |
| | ATTTATAAGTTGAGAAAGAAAAGAAAAAACCAAGAAAAAAAGAAGCAAAG | |
| 5 | TTTTGTATCTCTCTATATCTTGG | gRNA 4 of |
| | | ANTb |
| 6 | TTTCTTTGTGTCTCAGCTCAAGG | gRNA 2 of |
| | | ANTb |
| 7 | TGGGAAAAGATTCAGAACTCAGG | gRNA 1 of |
| | | ANTb |
| 8 | CTTGCCCGTCAAATCTATGGCCCCCTTCCACTGCACTGCTTGCAACCTTG | Promoter |
| | TCACCGTAAAAACAAAAATTAAATTAATAAACCCCATTTCTGTGGGGTAT | Region |
| | ATTTCTTTTTCAAAAACATTTCATGTACAATCGTTATACTCCAAGATATA | Sequence |
| | GAGAGATACAAAAAAATATAAGATTTAATAACATTTAAAATATGATAAGA | + 5′ UTR |
| | AAAGAGAAACGAGAGATGTTGGTCAGATATTTAAAATAAGTATTTTTATT | of ANTb |
| | TTGATGATCAAAAGTTTTAATAATTTGATTATTATTATTATTATTATTTA | |
| | TTTCTTTTCTTATCATCCTATATTATTAATGAAATCAATTATTTATTTCT | |
| | TATTTTTTTTTCTTTGTGTCTCAGCTCAAGGATTATTAAAAAAAATATTT | |
| | TTGTAGTGTAAATATAATATCATGAAAATATAAAAAGAATATTAAATAAA | |
| | CTTTTTTGGCTACCTGAGTTCTGAATCTTTTCCCAAATCCCCTCACCGTC | |
| | TATATCTTGGTCATTTGCTCACTAGATTCACCTACTAGCATTCAAGTTTT | |
| | CAGAAAATGACCAAAAAAAAGTTGATCTCACCGTTCCCATTTTGGGCTCT | |
| | TTCTTCTTATCCACTTTGAAATTAGATTCTGGGTTTAGCATAGGGTTTAA | |
| | GAAAAAGCTAAGTCTAGCTGGTGGGGGTGGTTTCTCTTTTCTTATTTTCT | |
| | TTATAAAAAAAGAAAAGAAAGAAGGCGAAAGACTTGTATTGTGAGTATAT | |
| | ATGCACACACATACACAGATCCCTATGAAATGAAGTGGGTGCTCCCAGCA | |
| | TCACTTGCTAGTAACACTTATTCCTTCTGCTTAGTTTAAAGAGACCAGTT | |
| | TTATTATGCTCGAGTGAAGAAAGAGAGAGAAAGAGAAGAAAGAGTTTTGG | |
| | TTGGTTGTTTTTCTTGTGTGAGAAAGAAAAAAGAAACAAAAAAACCTTCC | |
| | TGTAGCTAAACAACAAAAACCAAGTCTTCATTGGCAACAAGAAGATTATT | |
| | ATTTTGATATGATTTGTTTATTTGCCACCCAATGATTGACTTTGCCTAGC | |
| | TGCTGCTACGAGAGACGATATTCTGCTGGTGGTGGTGCTAGCAATATGCA | |
| | TCGGAGATATTTGTTTTTTTTTGCGTTGTTGTTCAATTAGAATTGGAGTT | |
| | TTACTTCGATAATTTATAATACATATTTAATATATTGTTGTAATGATACT | |
| | TCGATTCTGATTGAAAAACAACAAGAAAAAAAGTACATGCTAAACTGCAA | |
| | TTTTTTGTTTTAGTGCTTTTTTTTTCTTGTTTTGCAATTTGCAATATTAT | |
| | GGATCTTTAAGAGTGTTGTTCTTGTTGTGGCCTTATTGTTGAAGTG | |
| 9 | MDKKYSIGLAIGTNSVGWAVITDEYKVPSKKFKVLGNTDRHSIKKNLIGA | Engineered |
| | LLFDSGETAEATRLKRTARRRYTRRKNRICYLQEIFSNEMAKVDDSFFHR | protein |
| | LEESFLVEEDKKHERHPIFGNIVDEVAYHEKYPTIYHLRKKLVDSTDKAD | dCas9-TV: |
| | LRLIYLALAHMIKFRGHFLIEGDLNPDNSDVDKLFIQLVQTYNQLFEENP | SEQ ID |
| | INASGVDAKAILSARLSKSRRLENLIAQLPGEKKNGLFGNLIALSLGLTP | NO: 24 + |
| | NFKSNFDLAEDAKLQLSKDTYDDDLDNLLAQIGDQYADLFLAAKNLSDAI | SEQ ID |
| | LLSDILRVNTEITKAPLSASMIKRYDEHHQDLTLLKALVRQQLPEKYKEI | NO: 48 |
| | FFDQSKNGYAGYIDGGASQEEFYKFIKPILEKMDGTEELLVKLNREDLLR | |
| | KQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMTNFDK | |
| | NLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQ | |
| | LKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHP | |
| | VENTQLQNEKLYLYYLQNGRDMYVDQELDINRLSDYDVDAIVPQSFLKDD | |
| | SIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLI | |
| | REVKVITLKSKLVSDFRKDFQFYKVREINNYHHAHDAYLNAVVGTALIKK | |
| | YPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPE | |
| | DNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQ | |
| | SITGLYETRIDLSQLGGDKRPAATKKAGQAKKKKPRGGSGGLLDPGTPMD | |
| | ADLVASSTVVWEQDADPFAGTADDFPAFNEEELAWLMELLPQGGSGGLLD | |
| | PGTPMDADLVASSTVVWEQDADPFAGTADDFPAFNEEELAWLMELLPQAR | |
| | GGSGGLLDPGTPMDADLVASSTVVWEQDADPFAGTADDFPAFNEEELAWL | |
| | MELLPQGGSGGLLDPGTPMDADLVASSTVVWEQDADPFAGTADDFPAFNE | |
| | EELAWLMELLPQARGGSGGLLDPGTPMDADLVASSTVVWEQDADPFAGTA | |
| | DDFPAFNEEELAWLMELLPQGGSGGLLDPGTPMDADLVASSTVVWEQDAD | |
| | PFAGTADDFPAFNEEELAWLMELLPQARGGSGGGGSGGDALDDFDLDMLG | |
| | SDALDDFDLDMLGSDALDDFDLDMLGSDALDDFDLDMLARGSDALDDFDL | |
| | DMLGSDALDDFDLDMLGSDALDDFDLDMLGSDALDDFDLDML. | |
| 10 | MDKKYSIGLAIGTNSVGWAVITDEYKVPSKKFKVLGNTDRHSIKKNLIGA | Engineered |
| | LLFDSGETAEATRLKRTARRRYTRRKNRICYLQEIFSNEMAKVDDSFFHR | protein |
| | LEESFLVEEDKKHERHPIFGNIVDEVAYHEKYPTIYHLRKKLVDSTDKAD | for BPEp. |
| | LRLIYLALAHMIKFRGHFLIEGDLNPDNSDVDKLFIQLVQTYNQLFEENP | SEQ ID |
| | INASGVDAKAILSARLSKSRRLENLIAQLPGEKKNGLFGNLIALSLGLTP | NO: 24 + |
| | NFKSNFDLAEDAKLQLSKDTYDDDLDNLLAQIGDQYADLFLAAKNLSDAI | SEQ ID |
| | LLSDILRVNTEITKAPLSASMIKRYDEHHQDLTLLKALVRQQLPEKYKEI | NO: 46 |
| | FFDQSKNGYAGYIDGGASQEEFYKFIKPILEKMDGTEELLVKLNREDLLR | dCAS9 |
| | KQRTFDNGSIPHQIHLGELHAILRRQEDFYPFLKDNREKIEKILTFRIPY | (SEQ ID |
| | YVGPLARGNSRFAWMTRKSEETITPWNFEEVVDKGASAQSFIERMTNFDK | NO: 24) |
| | NLPNEKVLPKHSLLYEYFTVYNELTKVKYVTEGMRKPAFLSGEQKKAIVD | fused at |
| | LLFKTNRKVTVKQLKEDYFKKIECFDSVEISGVEDRFNASLGTYHDLLKI | its C |
| | IKDKDFLDNEENEDILEDIVLTLTLFEDREMIEERLKTYAHLFDDKVMKQ | terminus |
| | LKRRRYTGWGRLSRKLINGIRDKQSGKTILDFLKSDGFANRNFMQLIHDD | to an |
| | SLTFKEDIQKAQVSGQGDSLHEHIANLAGSPAIKKGILQTVKVVDELVKV | EAR- |
| | MGRHKPENIVIEMARENQTTQKGQKNSRERMKRIEEGIKELGSQILKEHP | repression |
| | VENTQLQNEKLYLYYLQNGRDMYVDQELDINRLSDYDVDAIVPQSFLKDD | domain |
| | SIDNKVLTRSDKNRGKSDNVPSEEVVKKMKNYWRQLLNAKLITQRKFDNL | (SRDX; |
| | TKAERGGLSELDKAGFIKRQLVETRQITKHVAQILDSRMNTKYDENDKLI | SEQ ID |
| | REVKVITLKSKLVSDFRKDFQFYKVREINNYHHAHDAYLNAVVGTALIKK | NO: 46; |
| | YPKLESEFVYGDYKVYDVRKMIAKSEQEIGKATAKYFFYSNIMNFFKTEI | dCas9- |
| | TLANGEIRKRPLIETNGETGEIVWDKGRDFATVRKVLSMPQVNIVKKTEV | SRDX) |
| | QTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPE | |
| | DNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQ | |
| | SITGLYETRIDLSQLGGDKRPAATKKAGQAKKKKGDGSGSGSGSGSVDLD | |
| | LDLEELRLGFALDLDLELRLGFALDLDLELRLGFA | |
| 11 | TGGAGAATGGTGCGCAGTGGG | gRNA 6 of |
| | | BPEp a |
| | | and b |
| 12 | AAGAGGAGTGACTTGGTTTTTGG | gRNA 4 of |
| | | BPEp a |
| | | and b |
| 13 | ACTCTCTCTACAAGTGGTAAAGG | gRNA 5 of |
| | | BPEp a |
| | | and b |
| 14 | CATTAGATGAGTGATATAAAAGG | gRNA4 of |
| | | BPEp a |
| | | and b |
| 15 | AGTTTCCGTGAATCAAAAATAGG | gRNA 4 of |
| | | BPEp a |
| | | and b |
| 16 | CACATGTTCTATTGTAAGATTGG | gRNA 2 of |
| | | BPEp a |
| 17 | CTTTTCGTAACCCAACCGCACTTGCTCCGAGTCTCAGATTCCGAACACAT | BPEpa |
| | TAAAACCCTTCTTACGTGGAACCATCCCATTGGCCAAACCAAATCAACGG | Promoter |
| | TCAAGATCTCGTAGAGTATGAGACAGGGTAATGCAATCGTACAGCAACGA | Region |
| | AGAGGGGACCACCAAAACCTCAGTGGAAACGACTTCCTACAATGCCACCT | Sequence + |
| | AGGTTCGTAGAACAAAAGTATATATATATATATATATATATATATATTTT | 5′ UTR |
| | TTTCCTTCTAAATCATGGCATTAGAATTACTATGTAAAAATTAAACTCTT | |
| | TCCAAATATTTAAGATAATTTCACTTACGTCCATATAACAGTAATATAAG | |
| | CGAAGAAAAATATAATATTTTTTTTTTAAAAAATACTCATACATTTTCTT | |
| | GTAATACTTTTAAAAATTTGTTAAAAATTATCAATATTATGAATCTCACT | |
| | CTTCTTAATTTAAATATAAGATCCATCAAAATAAATAATTTTGATGAATC | |
| | ATAAAAATTAAAAAGAACAAAAAAAAAAGGTTAGAAATAGTATGACTAGA | |
| | ATTCTTATTTTACTTTATTACCGTATTATTATTATTATTAGTATGTAACA | |
| | TTCATCAAACTTGTCAAAAGCTAATTTCTTCATCAGTAGAAAAACACAAC | |
| | TAATCTCTGATTCTTTCAGTCACCTATTTTTGATTCACGGAAACTTGAAC | |
| | TCGAAAAATTATTAAATAAGAACTCCTTTACCACTTGTAGAGAGAGTTAT | |
| | ACTATAAAGTATAAACCCACTGCGCGCACCATTCTCCACTGTCCAAGGAG | |
| | ATCAAGCACCAAACCCAACACACACCTCTCTCTCTCTCTCTCTCTCTCTT | |
| | GTGCTGTTGTTCTATTTCTCATTCTTTCGCAAGTGAATGTTGAACTGACT | |
| | CTGCGGTTCGGTTCGGTTTTTTTTTTTTCTGTTT | |
| 18 | ATAATTACAATAATGATATCATTATTGTCATGAATCTCTTAAAATAAATT | BPEpb 5′ |
| | GATGATAATTTAATGATTTAGTTATACAAGTACATAATATTAAAATTATT | UTR |
| | TTTGTTTTATCTTAAAATCTTAGTTATAAATAATTTTATTTCACTTAAAC | |
| | ACATTTTATTTTAAAATTTGTATTAAAAAATTAAACACTAAAAAGGATAC | |
| | AACCACCTCAAGTAGACTCTTAGAATTCTCATGTTAAAGTACTTGTTTGA | |
| | AGTAAAGAAGAATTAAATTTGATAAAATTAATTTCATAATGCTAATATTT | |
| | AATAAAAAATGCATATAAATTAATAGTAGTATCAATTACCAATCTTACAA | |
| | TAGAACATGTGTACATCAATTATCCAATAAAAAAAGACTTATAGATTTTA | |
| | AATGGGTTAAAAACTTAAAATACATTAGATGAGTGATATAAAAGGAAGAA | |
| | AAGAAAAAAGAGGAGTGACTTGGTTTTTGGGAGTCCCGAGGTGGGTGTCT | |
| | CTAAAACAGTGAGAAATTGACAGTAATGGTGGACAGACAAATACAGATAC | |
| | TACTATTCTTATTCTACTACTAGTTTCCAACGAAAGGAGATCACTATCCA | |
| | AACCATTCTCACTTTCTCCCTTCAATGCCACTCTTCTTCCTTTCTCTTTG | |
| | AGAGACTGCACACAAATTTCTACTGTTTCCAGAGCGACA | |
| 19 | atatttgaatatacttaatgttgatgttaaatattatttcatccatctta | DNA |
| | aaataagtgtcattctatttattttatatagacaaaaaaaagttaataaa | sequence |
| | ttaataaaaaaacagtaattttataaaactaaccttagaattaatatttc | of pAGL1 |
| | attaaaaaactaaaattacacttattagaaatattagaaaaaaaatatca | promoter |
| | ctactaaaaatattattttttatgacgaacattctaaaatggttatatga | |
| | aatcattttagaatgtcatgcgatgacaattgtaaatacaagattgttta | |
| | acaaagacgattttacaaaaattgtcttaaaaattattgttttatgacgg | |
| | tttgatgaaaaccgtcttaaaatgattgtcattctaaaacgattttcgta | |
| | aaatcctcttaaaagttagtgcttttaatttttatgtctccctctcctct | |
| | ctcatttctctccctctcactctctactacttcttccgttccaaaataat | |
| | tgttgtattaggttattttacataggtgaagtaataaatagatgaaatat | |
| | aatgataattttagaaaattaatcttatatcatcatttaatacatttata | |
| | tatagttcatcattaataaataaaaaaataattaatgttacatttaaaaa | |
| | ctaaaatgataaaagtaattaatatacattaagaaactaaaatgacatat | |
| | ttttcttatacgacaattataacacgatgagggagtaatgaatattagtt | |
| | aaaattagtttttgatcacctaatttataattttgattcaatttagtcct | |
| | ttagttattttgggttcaatttaatcctctaatttttaaaattatttttt | |
| | ttactttccattccatcacaagttccaaatgaatttgaatttaatcaact | |
| | tatggtagtatatatagaactttgaaagattattatctattgtaaaaaaa | |
| | ttcaatcttaagattttttttgcataacatgattcctaaactactcatca | |
| | ggtgtcttaaagatcagttactagtgtaatggagctagctacttggctca | |
| | catttcaggtattttacgtaaaagtccttacatgcactgtcgccctaaca | |
| | ctttgtttatgctaaccttgtcatcaaaatcaaaatgcagaaaagggaaa | |
| | agaaagaaaaaattcaaactggctttcccatccccataaagacttgctaa | |
| | atataaggagaagaagtgaagattatgatcagacatcacgtgggaaccct | |
| | tcttagttaaaattgtaataattcaagagagagggtcaagagtcactgga | |
| | aactctcaggtttcaattttcaactgtacaaaataaaaattaaagagatg | |
| | caggggtcagttaaaggaaataaatcttgtagatataaatatctatatat | |
| | agattatggttggataactttttatagaagaagaaaagaaaaacttgtct | |
| | tataaatttaaatcatcttacacatgcatgcacttgaattttatagaaac | |
| | tcttataagctaaatatataaattaattttaatttatgaaaatatatagt | |
| | ttgcttcattttactatttatttacttttgttataagtgtaaacaaggcc | |
| | atattcgaagtaaaaggccaaaaactcaaaatccaagaagcaaaatggtt | |
| | tctttattttttttatgctttaaaaatttgaagcttcggaattttgcgtg | |
| | caaacttttatgtgcatgtccctctcattgaaattgaatcaaaccccaca | |
| | ttaacaaattaaagtcactttcgaaaagaccctttgattatctccctctc | |
| | ctttttoccatacatgaagaaatggatggtttatggagcatgaaaggtga | |
| | acctcaagtctctctctctctcttctctctcatgcttaaattctccaaca | |
| | cgagagtaaattttagagtgagtggtaattgtggagggtctatttttctg | |
| | tcataaatacctgtctgctaggccaacccacttccatattttgcatcctt | |
| | tgtacctcagttccaggttaacac | |
| 20 | TATCGTATAGTAATTAGCTGTGG | gRNA 4 of |
| | | SUC9a |
| 21 | CATCATTTACGTAAACGTTTTGG | gRNA 6 of |
| | | SUC9a |
| 22 | AGGGTTGAAGAGTTTATATATGG | gRNA 3 of |
| | | SUC9a |
| 23 | atggataagaagtactccatcggactggctatcggaactaactccgtggg | dCas9 |
| | atgggctgtgatcactgatgagtacaaggtgccatccaagaagttcaagg | Sequence |
| | tgctgggaaacactgatagacactccatcaagaagaacctgatcggagct | + Soltu |
| | ctgctgttcgattccggagagactgctgaggctactagactgaagagaac | IV2 Intron |
| | tgctagaagaagatacactagaagaaagaacagaatctgctacctgcaag | |
| | agatcttctccaacgagatggctaaggtggatgattccttcttccacaga | |
| | ctggaggagtccttcctggtggaggaggataagaagcacgagagacaccc | |
| | aatcttcggaaacatgtggatgaggtggcttaccacgagaagtacccaac | |
| | tatctaccacctgagaaagaagctggtggattccactgataaggctgatc | |
| | tgagactgatctacctggctctggctcacatgatcaagttcagaggacac | |
| | ttcctgatcgagggagatctgaacccagataactccgatgtggataagct | |
| | gttcatccaactggtgcaaacttacaaccaactgttcgaggagaacccaa | |
| | tcaacgcttccggagtggatgctaaggctatcctgtccgctagactgtcc | |
| | aagtccaggagactggagaacctgatcgctcaactgccaggagagaagaa | |
| | gaacggactgttcggaaacctgatcgctctgtccctgggactgactccaa | |
| | acttcaagtccaacttcgatctggctgaggatgctaagctgcaactgtcc | |
| | aaggatacttacgatgatgatctggataacctgctggctcaaatcggaga | |
| | tcaatacgctgatctgttcctggctgctaagaacctgtccgatgctatcc | |
| | tgctgtccgatatcctgagagtgaacactgagatcactaaggctccactg | |
| | tccgcttccatgatcaagagatacgatgagcaccaccaagatctgactct | |
| | gctgaaggctctggtgagacaacaactgccagagaagtacaaggagatct | |
| | tcttcgatcaatccaagaacggatacgctggatacatcgatggaggagct | |
| | toccaagaggagttctacaagttcatcaagccaatcctggagaagatgga | |
| | tggaactgaggagctgctggtgaagctgaacagagaggatctgctgagaa | |
| | agcaaagaactttcgataacggatccatcccacaccaaatccacctggga | |
| | gagctgcacgctatcctgaggagacaagaggatttctacccattcctgaa | |
| | ggataacagagagaagatcgagaagatcctgactttcagaatcccatact | |
| | acgtgggaccactggctagaggaaactccagattcgcttggatgactaga | |
| | aagtccgaggagactatcactccatggaacttcgaggaggtaagtttctg | |
| | cttctacctttgatatatatataataattatcattaattagtagtaatat | |
| | aatatttcaaatatttttttcaaaataaaagaatgtagtatatagcaatt | |
| | gcttttctgtagtttataagtgtgtatattttaatttataacttttctaa | |
| | tatatgaccaaaatttgttgatgtgcaggtggtggataagggagcttccg | |
| | ctcaatccttcatcgagagaatgactaacttcgataagaacctgccaaac | |
| | gagaaggtgctgccaaagcactccctgctgtacgagtacttcactgtgta | |
| | caacgagctgactaaggtgaagtacgtgactgagggaatgagaaagccag | |
| | ctttcctgtccggagagcaaaagaaggctatcgtggatctgctgttcaag | |
| | actaacagaaaggtgactgtgaagcaactgaaggaggattacttcaagaa | |
| | gatcgagtgcttcgattccgtggagatctccggagtggaggatagattca | |
| | acgcttccctgggaacttaccacgatctgctgaagatcatcaaggataag | |
| | gatttcctggataacgaggagaacgaggatatcctggaggatatcgtgct | |
| | gactctgactctgttcgaggatagagagatgatcgaggagagacttaaga | |
| | cttacgctcacctgttcgatgataaggtgatgaagcaactgaagagaaga | |
| | agatacactggatggggtagactgtccagaaagctgatcaacggaatcag | |
| | agataagcaatccggaaagactatcctggatttcctgaagtccgatggat | |
| | tcgctaacagaaacttcatgcaactgatccacgatgattccctgactttc | |
| | aaggaggatatccaaaaggctcaagtgtccggacaaggagattccctgca | |
| | cgagcacatcgctaacctggctggatccccagctatcaagaagggaatcc | |
| | tgcaaactgtgaaggtggtggatgagctggtgaaggtgatgggtagacac | |
| | aagccagagaacatcgtgatcgagatggctagagagaaccaaactactca | |
| | aaagggacaaaagaactccagagagagaatgaagagaatcgaggagggaa | |
| | tcaaggagctgggatcccaaatcctgaaggagcacccagtggagaacact | |
| | caactgcaaaacgagaagctgtacctgtactacctgcaaaacggaagaga | |
| | tatgtacgtggatcaagagctggatatcaacagactgtccgattacgatg | |
| | tggatgcaatcgtgccacaatccttcctgaaggatgattccatcgataac | |
| | aaggtgctgactagatccgataagaacagaggaaagtccgataacgtgcc | |
| | atccgaggaggtggtgaagaagatgaagaactactggagacaactgctga | |
| | acgctaagctgatcactcaaagaaagttcgataacctgactaaggctgag | |
| | agaggaggactgtccgagctggataaggctggattcatcaagagacaact | |
| | ggtggagactagacaaatcactaagcacgtggctcaaatcctggattcca | |
| | gaatgaacactaagtacgatgagaacgataagctgatcagagaggtgaag | |
| | gtgatcactctgaagtccaagctggtgtccgatttcagaaaggatttcca | |
| | attctacaaggtgagagagatcaacaactaccaccacgctcacgatgctt | |
| | acctgaacgctgtggtgggaactgctctgatcaagaagtacccaaagctg | |
| | gagtccgagttcgtgtacggagattacaaggtgtacgatgtgagaaagat | |
| | gatcgctaagtccgagcaagagatcggaaaggctactgctaagtacttct | |
| | tctactccaacatcatgaacttcttcaagactgagatcactctggctaac | |
| | ggagagatcagaaaaagaccactgatcgagactaacggagagactggaga | |
| | gatcgtgtgggataagggaagagatttcgctactgtgagaaaggtgctgt | |
| | ccatgccacaagtgaacatcgtgaaaaagactgaggtgcaaactggagga | |
| | ttctccaaggagtccatcctgccaaagagaaactccgataagctgatcgc | |
| | tagaaagaaggattgggatccaaagaagtacggaggattcgattccccaa | |
| | ctgtggcttactccgtgctggtggtggctaaggtggagaagggaaagtcc | |
| | aagaagctgaagtccgtgaaggagctgctgggaatcactatcatggagag | |
| | atcctccttcgagaagaacccaatcgatttcctggaggctaagggataca | |
| | aggaggtgaagaaggatctgatcatcaagctgccaaagtactccctgttc | |
| | gagctggagaacggaagaaagagaatgctggcttccgctggagagctgca | |
| | aaagggaaacgagctggctctgccatccaagtacgtgaacttcctgtacc | |
| | tggcttcccactacgagaagctgaagggatccccagaggataacgagcaa | |
| | aagcaactgttcgtggagcaacacaagcactacctggatgagatcatcga | |
| | gcaaatctccgagttctccaagagagtgatcctggctgatgctaacctgg | |
| | ataaggtgctgtccgcttacaacaagcacagagataagccaatcagagag | |
| | caagctgagaacatcatccacctgttcactctgactaacctgggagctcc | |
| | agctgctttcaagtacttcgatactactatcgatagaaagagatacactt | |
| | ccactaaggaggtgctggatgctactctgatccaccaatccatcactgga | |
| | ctgtacgagactagaatcgatctgtcccaactgggaggagat | |
| 24 | MDKKYSIGLAIGTNSVGWAVITDEYKVPSKKFKVLGNTDRHSIKKNLIGA | Engineered |
| | LLFDSGETAEATRLKRTARRRYTRRKNRICYLQEIFSNEMAKVDDSFFHR | Protein |
| | LEESFLVEEDKKHERHPIFGNIVDEVAYHEKYPTIYHLRKKLVDSTDKAD | for dCas9 |
| | LRLIYLALAHMIKFRGHFLIEGDLNPDNSDVDKLFIQLVQTYNQLFEENP | |
| | INASGVDAKAILSARLSKSRRLENLIAQLPGEKKNGLFGNLIALSLGLTP | |
| | NFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEI | |
| | FFDQSKNGYAGYIDGGASQEEFYKFIKPILEKMDGTEELLVKLNREDLLR | |
| | KQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMTNFDK | |
| | NLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQ | |
| | LKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHP | |
| | VENTQLQNEKLYLYYLQNGRDMYVDQELDINRLSDYDVDAIVPQSFLKDD | |
| | SIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLI | |
| | REVKVITLKSKLVSDFRKDFQFYKVREINNYHHAHDAYLNAVVGTALIKK | |
| | YPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPE | |
| | DNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQ | |
| | SITGLYETRIDLSQLGGD | |
| 25 | gtaagtttctgcttctacctttgatatatatataataattatcattaatt | Soltu IV2 |
| | agtagtaatataatatttcaaatatttttttcaaaataaaagaatgtagt | Intron |
| | atatagcaattgcttttctgtagtttataagtgtgtatattttaatttat | |
| | aacttttctaatatatgaccaaaatttgttgatgtgcag | |
| 26 | CTCTCCCTCTGCTGCGAGTCAAATAAAGTGAGTTTTCTTGATTAAATTTG | Promoter |
| | ACACCATAGGCCCCAATCCCATGGTTGCCAATTGCTGTGCATAAACGAAC | Region |
| | GAAGGAAAAAGAAACGGAGAAAAAGGCACCTTTTCACCAAAGTAGTCGAC | Sequence |
| | ACAAAATATACTTCCTCTCTTCTTCTCCTAATACACTCCAAAATTTTGCC | + 5′ UTR |
| | TCCCTCAATCGTCATTGCTTCTAGAACATATACTTCCTATCTCTTTATCC | of SUC9a |
| | CCTGAAACAATCCCAAATTTTCTCCATATAATTTAGCCTAGGTTTCTAAT | |
| | TCAAGATAAATCAAATCCAACTCAACTATTAGAATCACTATATATATATA | |
| | TATATATATATTATCCAAAACGTTTACGTAAATGATGACATCCACAAAGA | |
| | TTAAATTTAGTAAAATGACCACAGTATGGGTTAGAATTAGGATACAAAAA | |
| | ATGCAGAAAGTAAACCCAACTTCGCCAATGGATAAACATAGCCATTCATA | |
| | ATTATCAATTAGTTATCCTTAAAGAACACATGCAACGTAAAAAATGGGTA | |
| | GTTTTTGCCACAGCTAATTACTATACGATACAAATCCTATTATTATGTTA | |
| | AATTTTGAACCACTCATCCCAATGTTAAAAGCCATATATAAACTCTTCAA | |
| | CCCTCTCACATTTACACACACTCCTCCTCCTCCTCCTCCTCGAGGCCTTC | |
| | ACAGAGAGAAAGAGAAAACATTTCAAAAAAAATAAAAAAAAATAAGCCTC | |
| | TTTCTCTCTCTGAATTTCTAAGCCTCTCTCACAACA | |
| 27 | TACGTTGCATGTGTTCTTTAAGG | gRNA 7 of |
| | | SUC9b |
| 28 | GGAGTGTGTGTAAATGTGAGAGG | gRNA 9 of |
| | | SUC9b |
| 29 | CCTAATTCTAACCGATATTGTGG | gRNA 12 |
| | | of SUC9b |
| 30 | gacgtcgttgtggttggtgctttccttacattctgagcctctttccttct | AtUBI10 |
| | aatccactcatctgcatcttcttgtgtccttactaatacctcattggttc | Promoter |
| | caaattccctccctttaagcaccagctcgtttctgttcttccacagcctc | Sequence |
| | ccaagtatccaagggactaaagcctccacattcttcagatcaggatattc | |
| | ttgtttaagatgttgaactctatggaggtttgtatgaactgatgatctag | |
| | gaccggataagttcccttcttcatagcgaacttattcaaagaatgttttg | |
| | tgtatcattcttgttacattgttattaatgaaaaaatattattggtcatt | |
| | ggactgaacacgagtgttaaatatggaccaggccccaaataagatccatt | |
| | gatatatgaattaaataacaagaataaatcgagtcaccaaaccacttgcc | |
| | ttttttaacgagacttgttcaccaacttgatacaaaagtcattatcctat | |
| | gcaaatcaataatcatacaaaaatatccaataacactaaaaaaattaaaa | |
| | gaaatggataatttcacaatatgttatacgataaagaagttacttttcca | |
| | agaaattcactgattttataagcccacttgcattagataaatggcaaaaa | |
| | aaaacaaaaaggaaaagaaataaagcacgaagaattctagaaaatacgaa | |
| | atacgcttcaatgcagtgggacccacggttcaattattgccaattttcag | |
| | ctccaccgtatatttaaaaaataaaacgataatgctaaaaaaatataaat | |
| | cgtaacgatcgttaaatctcaacggctggatcttatgacgaccgttaagg | |
| | aaattgtggttgtcggacgaagtccagtaataaacggcgtcaaagtggtt | |
| | gcagccggcacacacgagtcgtgtttatcaactcaaagcacaaatacttt | |
| | tcctcaacctaaaaataaggcaattagccaaaaacaactttgcgtgtaaa | |
| | caacgctcaatacacgtgtcattttattattagctattgcttcaccgcct | |
| | tagctttctcgtgacctagtcgtcctcgtcttttcttcttcttcttctat | |
| | aaaacaatacc | |
| 31 | ttcctaacacctggagaaccttttatgtacttcacaaccctcaatgctgc | TBS |
| | ttccaggtgtgactgtttgggttgctgcatgaattgactcagcacctgca | Insulator |
| | ctgcaaagctgatatctggccttgtgattgtcaagtagagaagctttccc | |
| | accagtctctgataggatcccacatectctaactctgcatcatcagtctt | |
| | tcctaagtgcttgtcatattctacagtagtcagcctttggttctgttcca | |
| | ttggggttgacactggtctgcagcctoccagaccaacacctgatatcagt | |
| | tccagtgcatacttcctctggttcagtaggattcccttttctgatctcag | |
| | cacctcaatgcctaagaagtattttagttctcccaaatctttcatcttga | |
| | aatgctgatgcagggttgcctttgcttctgaaatcaaaacattgctgctg | |
| | cctgttattaacagatcatccacataaatcaggattatgacaaggtcagt | |
| | cccctctcttttggtaaacaaggagtgatcataagcactttgcataaaac | |
| | cagcctgcataaggacagtggtaagcttgatgttccactgccttgatgct | |
| | tgctttaaaccatagagggattcaacagcctgcacactttggtttcccct | |
| | tggctgtgaaaaccctgaggcagagacatataaacttcttccatgaggtc | |
| | accttgtagaaaagcattgttgacatccatctggaaaaggaaccagccct | |
| | tggaagcagcaacagatatgacagcttttacagtgaccattitggccact | |
| | ggagaaaaagtttcatggtagtcaaggccttcttgctgagtgtatccctt | |
| | ggccactagccttgccttaaacctgtcaacttcaccattagctttgtatt | |
| | taattttgtacacccatttggaccctataggctgtttaccagggggtaaa | |
| | gggacaatctcccaggtgttattatcctcaagagcctgtatctcaaggga | |
| | catggcctccatccatttctcatcttgagctgcttctttgaaggatttag | |
| | gttcagtataagtggagaaagcactcaaataagcttgatagtgaacaggc | |
| | aagtgatcataggaaacatagttggctatagggtatggaacatccctaga | |
| | gcctttgttcagtgtcacaaagtccttgagccagatgggaggacctgcat | |
| | tgcgtttaggtctagtgtgcaggtttgctggaactagagagggatcagct | |
| | acagcagtatggtgctcaaattcagcatttgctaggtcaggctcagctga | |
| | ctcaactgactctgcaggagcatgcaggtggcctgaaggtgcagcatcag | |
| | ctgaagtgatatgattttgggtagaatgagtccccaaagtgatatcctca | |
| | ttggcctctacaatgtcaggaataagaactggagtatcatcatcatcagt | |
| | atgaaagctggaaggaaaaatatcattatacactagctgcaaagctgtat | |
| | cttcagagctaagtgaacctgctcgagtgaacatatcaggttcatgggag | |
| | atggactctttgaaagggaactgaaactctctgaaaactacatccctgct | |
| | cactatgatcaccttattatccaagtcatacaacctgtaacccttttgag | |
| | tctcagaataaccaatgaagatggttcgcttggctctaggtgcaagtttg | |
| | tcacctttgggcagtgttgctgcaaaagcaagacacccaaacactctcaa | |
| | gtgatcaagcttggcatgtttctgatagagtagttcatatggacatttgc | |
| | cttgtaagattggagtagggagtctattgatcaggtatacaacagtcttg | |
| | acacagtctccccaaaacctggtaggtacaccactctgaaacttaagtgc | |
| | ccttgccatctcaaggatgtgtctgtgctttctctccacaacaccattct | |
| | gttgtggtgtgtagggacagctactttgatgaacaatcccaagagaggcc | |
| | agcaactcattacaactt | |
| 32 | catctccactgacgtaagggatgacgcacaatcccactatccttcgcaag | 35S |
| | acccttcctctatataaggaagttcatttcatttggagag | CaMV 35 |
| | | S |
| | | Promoter |
| 33 | TAGTCGACACAAAATATACTTCCTCTCTTCTTCTCCTAATACACTCCAAA | Promoter |
| | ATTTTGCCTCCCTCAATCGTCATTGCTTCTAGAACATATACTTCCTATCT | Region |
| | CTTTATCCCCTGAAACAATCCCAAATTTTCTCCATATAATTTAGCCTAGG | Sequence |
| | TTTCTAATTCAAGATAAATCAAATCCAACTCAACTATTAGAATCACTATA | + 5′ UTR |
| | TATATATATATTATCCAAAACTTTTACGTAAATGATGACATCCACAAAGA | of SUC9b |
| | TTAAATTTAGTAAAATGACCACAATATCGGTTAGAATTAGGATACAAAAA | |
| | ATGCAGAAAGTAAACCCAACTTCGCCAATGGATAAACATAGCCATTCATA | |
| | ATTATCAATTAGTTATCCTTAAAGAACACATGCAACGTAAAAAATGGGTA | |
| | GTTTTTGCCACCATTAGCTAATTACTATACGATACAAATCCTATTATTAT | |
| | CTTAAATTTTGAACCACTCATCCCAATGTTAAAAGTCATATATAAACTCT | |
| | TCAACCCTCTCACATTTACACACACTCCTCCTCCTCCTTGAGGCTTTCAC | |
| | AGAGAGAAAGAGAAAACATTTCAAAAAAAATAAAAAAAAATAAGCCTCTT | |
| | TCTCTCTCTGAATTTCTAAGCCTCTCTCACAACA | |
| 34 | CACATGTCACACTCTGGTGCACATGGTGTTGGTAGAAAGACCAAATGTCA | W1 |
| | CGCCCATAACTATCAATCGTTGTCAGTATTGCTATATTCCTTGGGGATTT | promoter |
| | ACGTCTCATTGACAACCAGAGTGGTTTTAAGACAACTGTTTTTATCTAGC | |
| | TATGGAAGATTCTCACGATGCTCAAGAATTTGAAGTGATCAATGGGGTTT | |
| | GATGCAAGAGGTAATGTCCTGAGTTGGAGATTTGAGTTATTACGTCTCGT | |
| | ATAGGTCATTGAGGATTGAGGAGATCACACCGATATGATGCCCGAGGAGG | |
| | AGGAAGTGCATCATGAGCTTGTACGAGATTTAGAGCCTTCATCTTAGGAG | |
| | GAGGATTGATTACCTTCCTGTGTTTATACCTTAGGCGTAATTCAATACTT | |
| | TTAGTCGGGTAGTGATCTGCTCTGACCCTATATCCTTTTTTATATTTGAT | |
| | GTATTTGAGGGGTGAGATCTTCATTGAAACTGTTTTCTTTATGTAACAGA | |
| | TGTGTATCGATAATTTAATTACTTTTACACTTTGGGTTGTATCCAAATTT | |
| | CTTCTATTGTGACACTATTTATTTGATTTATTTACATATAAAGGTTTCAT | |
| | ATTATAAATTTGCGTTCATTTATTTACATATTTACTTACGACGTGAAAAC | |
| | TTGATGTTTCTTAAGCAATGCATTTATTTTATTTTATCTTACTTATACAA | |
| | AAAAAATGATGAGATATTTTTTTAACTTAAAAATAGAAATTAAAAGTTAA | |
| | TGACACGTATTCCACAATATTTATTATTCAAACCATTTTATGTAATATTT | |
| | AAAAAAAAAGTATATGAGATGTTACATAAAGAGACCATAAGTTGGAATAC | |
| | CTCCCACTAACAAAAAGTAACAAATAAATTACTAGGATTGGCCTTAAAAA | |
| | AATCGCGAGTTGTTAATTAATTTACTCCTTAATATTATAATAGTCAATTA | |
| | ATTAAATTGGTCCCTAAAATTAATTGTTTTCTTAAAAAAAATAAGTAAAA | |
| | CATGAGAATCCAAATCAATTTTAGTTGGTGGTTGGTTTGAGTATTTTTTT | |
| | TTCATTTTTAAAGAAAAATAAAATAAAAGTGAAAATACATTTTCTTAAAA | |
| | TTTAGAAAATATACATAAAAACAGATCAATAGTAAAGCAACGTTTTCAAT | |
| | CTTTTCTTAAGAAGTAAAAATACGGTGACACTTTGACATTTTTTCTCAAT | |
| | ATATTTTTTAAATCTTAAAAATTTAAAATAAAAAATATTTTTAAAAAATA | |
| | AAAATAAAAATAAAAAATAAACACTCAAACTAAAATAAGTAAAAGTTTTA | |
| | ATTTTCACAATGTGGCGCATTAAGACTCTCGAAATGCTATTTCCAAAAAG | |
| | AATGGTAGCTGACTAGATGCAATCTCTCGTAGGCGTTAGGTTGGCAATGG | |
| | AAGGTTTGGTTGCACGCTTGCACGCCCCATGTCAGCTGATAACCTACTTT | |
| | CTTAGAGTAACCCACTTTCCATATTCACAAATGATTAATGGTCAATCTTC | |
| | TAATTCACGCACTACTCACCCCAACCATCCATTAGGATATTCCGAGTCTA | |
| | ATGGCTCCATTATTAAAAAAAAAAAATCTTTTTATTTAAATTTTAACTTT | |
| | TCATTTTTATATATTTATTCTTAAACGAAAAATCTAAACTACTATATTTG | |
| | ATTCATATTCTTTATTTATTTTTTAAAATTTCCCCATTGTTTAAATTTTT | |
| | TAAATCAAAATCAAAATCATTTAAGTTTACTTAATTTTAATATTGTATGA | |
| | AATAATTAAAATTAAAAAAAAATGTTTTTTTAACAAAGTAAAAAGAAATT | |
| | ATAATGGTGATAGAACCACGGGAAAGTATTTGTGCAAAATAATTGAAATT | |
| | ACAAGTATTATTTTCTATTACAATTTTTTTAATTGTTTCTTTGCTTTTTT | |
| | TTTTTTTTAACTTCTATTTTGCAAGCATGTATTTTATATTTAATGTGTGT | |
| | GGAGGGGGACGGAGTACAAACTCTTTTTTGTCTATATTCAGTAAAATGTT | |
| | GGTGGAGACGTGCCTTGCGTGATGAACTACCATTTCAAACACCACACGAC | |
| | ACCACACATCCATTTTAAATATAACCCCATCATAGATATCCCGAATCATC | |
| | AAATTATTACTTCATAGCAACTAGCAAATTAATTAGCTTCACC | |
| 35 | ATGGACTCATTGTTACTTCTAAAAGAAATTGCCACTTCCATTTTGATCTT | W1 CDS |
| | CTTGATCACTCGTCTCTCCATTCAAACATTCCTCAAAAGCTATCGCCAGA | |
| | AACTCCCACCGGGGCCAAAAGGGTGGCCAGTTGTGGGTGCACTCCCTCTC | |
| | ATGGGAAGCATGCCTCATGTCACCTTAGCAAAGATGGCAAAAAAATATGG | |
| | ACCTATAATGTACCTCAAAATGGGCACTAACAACATGGTTGTGGCCTCTA | |
| | CTCCAGCTGCTGCTCGTGCCTTCCTCAAAACCCTTGATCAAAACTTTTCA | |
| | AACCGGCCCTCCAATGCTGGTGCAACCCATTTGGCTTATGATGCACGGGA | |
| | TATGGTGTTTGCTCATTACGGATCACGGTGGAAGTTGCTAAGAAAACTAA | |
| | GTAACTTGCACATGCTTGGAGGAAAGGCACTTGATGATTGGGCCCAAATT | |
| | CGAGATGAAGAGATGGGGCACATGCTTGGTGCAATGTACGATTGTAACAA | |
| | GAGGGATGAGGCTGTGGTGGTGGCGGAGATGTTGACATATTCAATGGCCA | |
| | ACATGATTGGCCAAGTTATATTGAGTCGTCGAGTGTTTGAGACAAAGGGT | |
| | TCGGAGTCTAACGAGTTCAAGGACATGGTGGTTGAGCTCATGACCGTTGC | |
| | TGGTTACTTCAACATTGGTGACTTCATACCCTTTTTGGCCAAGTTGGACT | |
| | TGCAAGGCATAGAGCGTGGCATGAAGAAGTTGCACAAGAAGTTTGATGCG | |
| | TTGTTAACGAGCATGATTGAGGAGCATGTTGCTTCTAGTCACAAGAGAAA | |
| | GGGCAAGCCCGATTTCTTAGACATGGTAATGGCTCATCATAGTGAGAACT | |
| | CCGATGGGGAGGAACTATCGCTCACCAACATCAAGGCACTACTCTTGAAC | |
| | CTATTCACCGCAGGCACCGATACATCTTCAAGTATAATAGAGTGGTCCTT | |
| | AGCCGAGATGTTGAAGAAGCCCAGCATAATGAAGAAGGCTCATGAAGAAA | |
| | TGGACCAAGTCATAGGAAGGGATCGCCGTCTCAAAGAATCTGACATACCA | |
| | AAGCTTCCCTACTTCCAAGCCATTTGCAAAGAGACCTATAGAAAGCACCC | |
| | TTCAACACCCCTAAACCTGCCTCGAATCTCATCTGAACCGTGCCAAGTGA | |
| | ATGGTTACTACATTCCCGAGAACACTAGGCTGAATGTGAACATTTGGGCC | |
| | ATAGGAAGAGACCCTGATGTGTGGAACAATCCTTTGGAGTTTATGCCCGA | |
| | GAGGTTTTTGAGTGGGAAGAATGCCAAAATTGACCCACGTGGGAATGATT | |
| | TTGAGCTTATTCCATTTGGTGCTGGGAGGAGGATTTGTGCAGGGACTAGG | |
| | ATGGGGATTGTGTTGGTTCACTACATTTTGGGCACTTTGGTGCATTCGTT | |
| | TGATTGGAAGCTACCCAATGGGGTGAGGGAGTTAGACATGGAGGAGTCCT | |
| | TTGGGCTTGCCTTGCAAAAAAAGGTTCCACTTGCTGCTTTGGTTACCCCT | |
| | AGGTTGAACCCAAGTGCTTACATTTCTTAGAATTGGTTGGGTTCGAATAT | |
| | TCACCAGCTATGTTCTCTAGCCTTATTTTGTTGTCCAATGATTTTGTGGC | |
| | TGTGGCTACATAAATAAGTAATGTTTGGGTTGCACAACCTATTTGTATTT | |
| | GTAAGGTTCTATGTTACTTGGAAATCCGTTACCCCACCACCTGCAAGGCT | |
| | TAGATTTTTATTCTTCATGGATCTCTAATTTAGCTGTCTTGTTTTGGTTT | |
| | AATTATATTTTTAATTCTCACTCA | |
| 36 | TGTTCAAAAATAAAAAGTGATTTTGAGATATAATATACATACGACAATTA | AP3 |
| | ATACAATATTATTTTTGTGTCTATCCCTCTCTTAGACCCTATTATTTATC | Promoter |
| | ACATTTGAAATTCAGAATTTTCTTTTTCTCTCGTTGTTGTACAAATATCT | Region |
| | AATCGTTAATTATTATTAATGCATTAAATATTTAAAATATAATTAATGAT | Sequence |
| | ATTAATTTTCCTAGCGTAAGACTAATAATAAATATCGACATTATGCGAGA | + 5′ UTR |
| | TTATGACATGATGTTATGTTAATAGTACAGAATAAAGTAAAAAAATATAT | |
| | ATAACAATAATAATTTAAAATATTTAAAATGACATATAAAGTAGTTTTCT | |
| | AAAACTTCAGTGATTAGTTCAATTTATTATTTCGCAACAATTATGATGGT | |
| | TAATTAAGGTGAATTAGAAGAAGAACTGAAAATTAAGATAATTAAGGAGA | |
| | ACAAATGAGCACTTCAAAATTTGCTATTAAGCCGTCTCTGCTAGCCATGC | |
| | ATGTTGGATGTAACTTCACCCGATTGATGGGTCCTTAATTATAATTTTTC | |
| | TTATGATAAAAAATAAATTGCTACTAAGGCGTACTGAACTATTCGAAATG | |
| | ACAACACTACAAGGCCTCGACAACGTTAACTTTTTTCCAATTTAGTGGGG | |
| | CGTTACAAGCTAAAGCTTTTTTATTTTTGAACTTTAAATTGCTCTTTCAC | |
| | TTTCATTTAATAACGCCTAAACCTAGCTACGAGCAATATAACCTGGGTGT | |
| | ATATAGCTATATATATCATATGCAGAGCAATCTGTAACCCCAAGGATAAA | |
| | ACGTTGTGTTGCTTATTATAGAAATCACAGTTTTAAATGGAACGAGTGAC | |
| | AGAGTTTGTTTGTTTCTAAATAGGATAGGAGGCATTTAATCGGAAATTGG | |
| | ACAGGAGAAAGTTGTTTTCTTTCTGCAAATTATGCCTCCAGAACTTGTTT | |
| | TACGGTAAGAAAATTTACACCAAATTTACAAGTATTGGTTCTGAACGCTA | |
| | CTTGCGGAAATTTTATGCCGCCAGTGACTTCTTGGCACTTTAAATAAGTT | |
| | AATTAGTTAATCATTTGAAAATATATTAACAAGAAGAATTTTTTTCTAGT | |
| | GTAATCAAGAGGTATTTTAAGCTTCTATCTTACACTCAATTAATCTTCTA | |
| | TTCTCCTATTGATTGGTCTATTTTAAAGTAAAGTAATATATTCAATTACG | |
| | ACTTCATGCTAAATTCAAAGATTATAACAGTAAAAACAATTTTTATTCAC | |
| | CTCTATTCAAACTGCCAGTTTAGCGCAAGTGTTTATTTTTCTGATAAACT | |
| | TGCGGCCGCACAAAACCCAGCATTGCTTCAATGTGCATAAGCATGGGTAC | |
| | GGTGGTGGTGTTTCTCCAACCTCCATTTCAGATTGTCTTGATTTGTAATT | |
| | CAAACACACACTTAGTCTTAAAATCATATTTTCTCATATCTCAATTGTGA | |
| | TTTTTCCAAATGCGTTGTGTGTAGGCGAAGCTAAGTTTAGGTTATTGCTC | |
| | AAGTGCCACAAGTTTGAGAGGGTTTTACTGTCTTTTTTATTTAATCTAGA | |
| | AATATGATTTTTGGACACGTCTACTTGTTACATTCCGTTTACTTATGGTA | |
| | GTATTTGACTAATAAAGACATTAAGATTATAGTAGCATTTGACTAATAAA | |
| | GACACGCGTACGTAGGTCACTATTTTATTGGCTAAGTAAAAAAAAAAAAA | |
| | AACACGAAAAAACTTTCAAACAAAGGGGAAGCGGAGAAATCAGCCTAACT | |
| | TGATATTAGCTGTACAAAATACATAAATAGCTGTCGGAAATAATTATCTC | |
| | TTGCAGTATATATATAGAAAGTAGCAGACTCTAAAATATAACATGAAGAT | |
| | GTGATTAAGCATTTATTCCATTATATGGCCCATAAACATGCATGATGCAA | |
| | ATAGAAGGATGTAGGGTAAAGTTGCCCTAAGAAACGAATTGAACTGCGGT | |
| | CGTTGCGGTATGAGAAAGAGAGAGAGAAAAGTGAGTGAAGGAAGGTGGGA | |
| | GAATGGTTTCCGTGACGGAGTAGGACGGAAAGTAAGGGTCCCCACAGTGC | |
| | ACATGGGAGCGGATTTGACGATGAGACGAGAAAGAGCACAAGACGTTTAT | |
| | GACAAAGCCTGCAATAGCTTTCGTTAATTTGGCAAAAGACCCCTACCATT | |
| | TGCGGCAATGCACCTCCCCGTTCTATTATTTCTCTTTCCCTCATTCATTA | |
| | TATCCCCTATTCTCTGCCCCAGATCGATCGAACAAGTGAATTAGCTAGAA | |
| | GCATTGAAGGACGTAGCTGCT | |
| 37 | CAAACAACACATATAGTGCAGATTCATAAGGTATAGACTTTGTTAATTTG | AGL 1 |
| | CACCCTCTCTTACCCTAAATCTTGTGTCTATTGTATTTTATCTCTTTCCA | Promoter |
| | TATTTGAATATACTTAATGTTGATGTTAAATATTATTTCATCCATCTTAA | (5′ UTR |
| | AATAAGTGTCATTCTATTTATTTTATATAGACAAAAAAAAGTTAATAAAT | Incl) |
| | TAATAAAAAAACAGTAATTTTATAAAACTAACCTTAGAATTAATATTTCA | |
| | TTAAAAAACTAAAATTACACTTATTAGAAATATTAGAAAAAAAATATCAC | |
| | TACTAAAAATATTATTTTTTATGACGAACATTCTAAAATGGTTATATGAA | |
| | ATCATTTTAGAATGTCATGCGATGACAATTGTAAATACAAGATTGTTTAA | |
| | CAAAGACGATTTTACAAAAATTGTCTTAAAAATTATTGTTTTATGACGGT | |
| | TTGATGAAAACCGTCTTAAAATGATTGTCATTCTAAAACGATTTTCGTAA | |
| | AATCCTCTTAAAAGTTAGTGCTTTTAATTTTTATGTCTCCCTCTCCTCTC | |
| | TCATTTCTCTCCCTCTCACTCTCTACTACTTCTTCCGTTCCAAAATAATT | |
| | GTTGTATTAGGTTATTTTACATAGGTGAAGTAATAAATAGATGAAATATA | |
| | ATGATAATTTTAGAAAATTAATCTTATATCATCATTTAATACATTTATAT | |
| | ATAGTTCATCATTAATAAATAAAAAAATAATTAATGTTACATTTAAAAAC | |
| | TAAAATGATAAAAGTAATTAATATACATTAAGAAACTAAAATGACATATT | |
| | TTTCTTATACGACAATTATAACACGATGAGGGAGTAATGAATATTAGTTA | |
| | AAATTAGTTTTTGATCACCTAATTTATAATTTTGATTCAATTTAGTCCTT | |
| | TAGTTATTTTGGGTTCAATTTAATCCTCTAATTTTTAAAATTATTTTTTT | |
| | TACTTTCCATTCCATCACAAGTTCCAAATGAATTTGAATTTAATCAACTT | |
| | ATGGTAGTATATATAGAACTTTGAAAGATTATTATCTATTGTAAAAAAAT | |
| | TCAATCTTAAGATTTTTTTTGCATAACATGATTCCTAAACTACTCATCAG | |
| | GTGTCTTAAAGATCAGTTACTAGTGTAATGGAGCTAGCTACTTGGCTCAC | |
| | ATTTCAGGTATTTTACGTAAAAGTCCTTACATGCACTGTCGCCCTAACAC | |
| | TTTGTTTATGCTAACCTTGTCATCAAAATCAAAATGCAGAAAAGGGAAAA | |
| | GAAAGAAAAAATTCAAACTGGCTTTCCCATCCCCATAAAGACTTGCTAAA | |
| | TATAAGGAGAAGAAGTGAAGATTATGATCAGACATCACGTGGGAACCCTT | |
| | CTTAGTTAAAATTGTAATAATTCAAGAGAGAGGGTCAAGAGTCACTGGAA | |
| | ACTCTCAGGTTTCAATTTTCAACTGTACAAAATAAAAATTAAAGAGATGC | |
| | AGGGGTCAGTTAAAGGAAATAAATCTTGTAGATATAAATATCTATATATA | |
| | GATTATGGTTGGATAACTTTTTATAGAAGAAGAAAAGAAAAACTTGTCTT | |
| | ATAAATTTAAATCATCTTACACATGCATGCACTTGAATTTTATAGAAACT | |
| | CTTATAAGCTAAATATATAAATTAATTTTAATTTATGAAAATATATAGTT | |
| | TGCTTCATTTTACTATTTATTTACTTTTGTTATAAGTGTAAACAAGGCCA | |
| | TATTCGAAGTAAAAGGCCAAAAACTCAAAATCCAAGAAGCAAAATGGTTT | |
| | CTTTATTTTTTTTATGCTTTAAAAATTTGAAGCTTCGGAATTTTGCGTGC | |
| | AAACTTTTATGTGCATGTCCCTCTCATTGAAATTGAATCAAACCCCACAT | |
| | TAACAAATTAAAGTCACTTTCGAAAAGACCCTTTGATTATCTCCCTCTCC | |
| | TTTTTCCCATACATGAAGAAATGGATGGTTTATGGAGCATGAAAGGTGAA | |
| | CCTCAAGTCTCTCTCTCTCTCTTCTCTCTCATGCTTAAATTCTCCAACAC | |
| | GAGAGTAAATTTTAGAGTGAGTGGTAATTGTGGAGGGTCTATTTTTCTGT | |
| | CATAAATACCTGTCTGCTAGGCCAACCCACTTCCATATTTTGCATCCTTT | |
| | GTACCTCAGTTCCAGGTTAACAC | |
| 38 | GAATTCGTTTCCATAGCCTAAGGCCACGAGTGCACGTGTTCATAGGCTAG | TA29a |
| | TTGTGTTAACCTTGGGGAGCAGTCGACAATTTGGATTGCATCGAGGTCTT | promoter |
| | GCCAAATTTTACTTTCAAGACAGTGATTTCGACGGCACAATAAACTTCCA | |
| | ATTAGTTATCTTCTCTTTTGTTCTGTATTGTGTTTTTTTCTATTTTTCTA | |
| | ACAATGGAAATAGTATATACCGGCCAACAGTGTTGCACTGTTGTCTGGAC | |
| | CTTGTCAAAGGTTCGACAGTGCATAGTTTGGTCTCGATGTGGCATATGGG | |
| | GGTTCTGATAAGTGATGAGATTATGAAAATAATTTAATTTTAGCTATTAA | |
| | AAATACAATACAACTTTCAAAAGCGGGAAACTTGTTTGATTATGATCTAG | |
| | GATTCTAGGTCTTTTTTTTTAAGTTCGAGACAGAAAACCAAAAACAAAAC | |
| | AGCTAATAAGAAGAAGAAAAACAATACAGCAATATTCCCACAAAAATAAA | |
| | TTTGTTCTTTTGCGTACTAGCCATAGGATTTAATTATATATATGGGCCAG | |
| | TCCAGAAGCATTGTCACTGATAGATCATAGACTAGTAAAAAAGAATGAAC | |
| | CACTTCAGAACACTAACTATATATATAAAGATAGATTCATATCTATTTAG | |
| | ATCGTGATTATTAATACAAGAAAATATTTGTATTTCTTAATTTTTTGTTT | |
| | TTAAATTTATGTAAATATATTGACATCTTTTTTAACGAAAATTCAAATTT | |
| | TAATTCATTATATTACAAGAAATTAACACATTTTACTAGACCAACTCTCA | |
| | TGTTACAACACTATATATGTTGAACATTACAAGAATGCCACTTTATCTCA | |
| | CCCCTTTAATTAGGAAAAGCAAAAATCTATTTGCATATAATTTTGTACCA | |
| | AAAAATTATACAATAAAAAAGAAAAAGAGAAAAGTAAAAGATAAGACGAC | |
| | TGATGTAATAAAAAGAGAGAAAAAATAATACTAAAAAATAATATAAATAA | |
| | CATAACTCTTAAAAAAAAAAGGTAACACTGTTGAGTATTTTTTTTATTAC | |
| | TTTTCTCTAAAAAGGTATCATGGTTTCACTAATGTCCAGGTTTTTTTTTT | |
| | CCAAATAAACTCCTTTCCGGTGGTAGGTATCGTTTCACACTTGAATACTT | |
| | TTTTGCAATATTTTTCGTAACACTTTATTTCTCTCCATTACATAATTTTA | |
| | CCCTTGCCTATGTCTTTATTCTGCCACATATACCCTAAGAGTGAGCAAAT | |
| | GTGTGCACGCATATAACCTGATTGGACTATATATCGGACTTGATACAAAT | |
| | GTGTGCTCACATGAAGAGAAAAAGGATTGAACCCATGATCATCATCATGT | |
| | CACATGCAGATGCCATGCATTCAACACCAAAGTTATGATCAGAAAACCTC | |
| | TCAAGGTAGGTGACTCACCAAATGAACAAGCCAATTTTTTGCTTCCCAAA | |
| | TTTCACCATAAATAGGGACACTACTCCATCCAAACCATACACGACCACAT | |
| | ACAGTGGGATTAGTAAGTAAACAAATTAATTAATAACCAAAAGTTATAAT | |
| | TTCAATTGAATTTAGTC | |
| 39 | TTTGTAATATTATGATTTACGTAAAAATCTATCTAATGATTTTATTTAAA | TA29b |
| | CTATAGTTTTGAACTTTTCCAACTGAAATTTCAACACAGTAATTATGGTT | promoter |
| | GCCTGTAACTCATATTTTGCAAGCCCCACTTCAATTATGCAGCCTAACCC | |
| | AAAAGTGGGGATATGTGATTTCAAAGGATATATGCATCAATCATTTCTCC | |
| | AAATTAGGAAAGTAATTTATATAAAATTAACTTGACTTTAAGTTAGAACT | |
| | GCATTTTACTGAATAAAAGTTATAATTATTAATAAATAAATGGTTAAGAT | |
| | TTTAACGTTATAATATATTCATACAGTACAAATTCAAGTTTTTATTCTTG | |
| | AAATAAAATTTTTATTTTAGGTATTTCATTTTTATGCAGTCATGTGTCTT | |
| | TTTTTTATTTTTGAAACATATTTTATGGTCATTTATATCAATTCAAATTA | |
| | GAATTTTCTTCCTCTCTAACTAATTATTCAACAACAAAAAAAAACTATTT | |
| | TTTCAAGTTTTTATTTTACTCAGTGTTTTAATCAATATTAAGCATAATGA | |
| | GTATTTATAAATCATTCTCAAAATAATATTTTACATGTTAAATAATTTTT | |
| | AAGTATTTTTAAGAGTGTGTTTAATTGTGTTTTAATTTCTTAGTCTATTT | |
| | TTTAAAAACTATTTTATGAAACATTTTTCATAAACTGATGTTTAGAGAGT | |
| | AAAAGTGGAAAAGTAAGATGTGAAAATACAAGGAAGAACATGAATAATAG | |
| | AAAATTAAAAGTAAAAAACCCAAACATCGATCATAGAGATGTTATAGTTT | |
| | TTTGGTTTTGAAAAAACTAAAAAAAGAGATAGTAGTTTTAAAAACAGTTT | |
| | TCGAAACTATTTTTATCAAACAACATAATTTTTTTAGCTTTTAAATAAAA | |
| | ACAGCTTTAAAAAATGTCCAGGTTTTTTTCCAAATAAATTCAATTCCATA | |
| | TTTTAATAAGTAATGGTTAATCCAGTGGTAGGTATATATTGTTTAACACT | |
| | TGAAATTTTTTTCCATTCAATTTTTTTAATATTTTTCATAACACTTAATT | |
| | TATTTATTACATATATCATATGTTATATTATTATTTTTCTCTATTCTCTT | |
| | CCTTTCTCTCTAGTTATTTGTAAAAAAAAAAACATATAATTTATTTCCAT | |
| | TGCATAAATTTTATCCATGTCTATCCATGCCTATGTTTTTATTCTGCCAC | |
| | ATATACCCTAAGTGTTAGCAAATATGTGCACACATTAACCTAATTAAACT | |
| | ATACTAGAACTATATATCGGACTTGATAGAAAGTGGAGATAAGCCAAGGA | |
| | AGACAAGAAAAAGGATTGAACACATGATCATCCTCATGTCACATGCAGTT | |
| | GCCATGGATCCAACACCAAAGTTATGATCAGAAAACCTCTCAAGGCAGGT | |
| | GACTCACGAAATGAACAAGCCAATTTGCTTCCAAAATTCACCATAAATAG | |
| | GGACACTACTCAATGCAAACCATGCATACACAATCATATACAGTGGGATT | |
| | AGTAAACAAATTAATTAATAACCAAAAGTTATTTAAATTGAAGTC | |
| 40 | MMKMEGIALKKRLSWISVCLLVLVSAAGMLFSTAAKTETSSHKAHTEAQV | Barnase |
| | INTFDGVADYLQTYHKLPDNYITKSEAQALGWVASKGNLADVAPGKSIGG | |
| | DIFSNREGKLPGKSGRTWREADINYTSGFRNSDRILYSSDWLIYKTTDHY | |
| | QTFTKIR | |
| 41 | MKKAVINGEQIRSISDLHQTLKKELALPEYYGENLDALWDCLTGWVEYPL | Barstar |
| | VLEWRQFEQSKQLTENGAESVLQVFREAKAEGCDITIILS | |
| 42 | TTTGTAATATTATGATTTACGTAAAAATCTATCTAATGATTTTATTTAAA | Barnase |
| | CTATAGTTTTGAACTTTTCCAACTGAAATTTCAACACAGTAATTATGGTT | expression |
| | GCCTGTAACTCATATTTTGCAAGCCCCACTTCAATTATGCAGCCTAACCC | construct; |
| | AAAAGTGGGGATATGTGATTTCAAAGGATATATGCATCAATCATTTCTCC | TA29b |
| | AAATTAGGAAAGTAATTTATATAAAATTAACTTGACTTTAAGTTAGAACT | promoter |
| | GCATTTTACTGAATAAAAGTTATAATTATTAATAAATAAATGGTTAAGAT | with |
| | TTTAACGTTATAATATATTCATACAGTACAAATTCAAGTTTTTATTCTTG | Barnase |
| | AAATAAAATTTTTATTTTAGGTATTTCATTTTTATGCAGTCATGTGTCTT | CDS. |
| | TTTTTTATTTTTGAAACATATTTTATGGTCATTTATATCAATTCAAATTA | In plasmid |
| | GAATTTTCTTCCTCTCTAACTAATTATTCAACAACAAAAAAAAACTATTT | in SEQ ID |
| | TTTCAAGTTTTTATTTTACTCAGTGTTTTAATCAATATTAAGCATAATGA | NO: 59 |
| | GTATTTATAAATCATTCTCAAAATAATATTTTACATGTTAAATAATTTTT | and SEQ |
| | AAGTATTTTTAAGAGTGTGTTTAATTGTGTTTTAATTTCTTAGTCTATTT | ID NO: |
| | TTTAAAAACTATTTTATGAAACATTTTTCATAAACTGATGTTTAGAGAGT | 102. |
| | AAAAGTGGAAAAGTAAGATGTGAAAATACAAGGAAGAACATGAATAATAG | These are |
| | AAAATTAAAAGTAAAAAACCCAAACATCGATCATAGAGATGTTATAGTTT | likely the |
| | TTTGGTTTTGAAAAAACTAAAAAAAGAGATAGTAGTTTTAAAAACAGTTT | same |
| | TCGAAACTATTTTTATCAAACAACATAATTTTTTTAGCTTTTAAATAAAA | plasmid. |
| | ACAGCTTTAAAAAATGTCCAGGTTTTTTTCCAAATAAATTCAATTCCATA | |
| | TTTTAATAAGTAATGGTTAATCCAGTGGTAGGTATATATTGTTTAACACT | |
| | TGAAATTTTTTTCCATTCAATTTTTTTAATATTTTTCATAACACTTAATT | |
| | TATTTATTACATATATCATATGTTATATTATTATTTTTCTCTATTCTCTT | |
| | CCTTTCTCTCTAGTTATTTGTAAAAAAAAAAACATATAATTTATTTCCAT | |
| | TGCATAAATTTTATCCATGTCTATCCATGCCTATGTTTTTATTCTGCCAC | |
| | ATATACCCTAAGTGTTAGCAAATATGTGCACACATTAACCTAATTAAACT | |
| | ATACTAGAACTATATATCGGACTTGATAGAAAGTGGAGATAAGCCAAGGA | |
| | AGACAAGAAAAAGGATTGAACACATGATCATCCTCATGTCACATGCAGTT | |
| | GCCATGGATCCAACACCAAAGTTATGATCAGAAAACCTCTCAAGGCAGGT | |
| | GACTCACGAAATGAACAAGCCAATTTGCTTCCAAAATTCACCATAAATAG | |
| | GGACACTACTCAATGCAAACCATGCATACACAATCATATACAGTGGGATT | |
| | AGTAAACAAATTAATTAATAACCAAAAGTTATTTAAATTGAAGTCTAATT | |
| | TCAATTGAATTTAGTCATGATGAAAATGGAGGGCATAGCTTTGAAGAAGC | |
| | GTCTGTCTTGGATTAGCGTATGTTTGCTCGTATTGGTGTCTGCCGCAGGA | |
| | ATGCTGTTCAGCACTGCTGCTAAAACAGAGACCTCATCCCATAAAGCTCA | |
| | TACTGAGGCTCAAGTGATAAATACCTTCGATGGGGTCGCTGACTACCTTC | |
| | AGACCTACCACAAATTGCCCGACAATTACATTACCAAGAGCGAGGCCCAA | |
| | GCTCTGGGGTGGGTTGCCTCTAAGGGAAATCTCGCAGATGTGGCCCCTGG | |
| | AAAATCAATTGGCGGCGACATATTTTCTAACAGGGAAGGCAAACTGCCCG | |
| | GTAAGAGTGGGCGTACATGGCGTGAAGCAGATATTAACTATACATCAGGC | |
| | TTTAGAAATAGTGACCGTATTCTTTATTCTTCAGACTGGTTGATTTACAA | |
| | GACCACTGATCATTATCAAACATTCACAAAGATCCGT | |
| 43 | GAATTCGTTTCCATAGCCTAAGGCCACGAGTGCACGTGTTCATAGGCTAG | Barstar |
| | TTGTGTTAACCTTGGGGAGCAGTCGACAATTTGGATTGCATCGAGGTCTT | expression |
| | GCCAAATTTTACTTTCAAGACAGTGATTTCGACGGCACAATAAACTTCCA | construct |
| | ATTAGTTATCTTCTCTTTTGTTCTGTATTGTGTTTTTTTCTATTTTTCTA | 1; TA29a |
| | ACAATGGAAATAGTATATACCGGCCAACAGTGTTGCACTGTTGTCTGGAC | promoter |
| | CTTGTCAAAGGTTCGACAGTGCATAGTTTGGTCTCGATGTGGCATATGGG | with |
| | GGTTCTGATAAGTGATGAGATTATGAAAATAATTTAATTTTAGCTATTAA | Barstar |
| | AAATACAATACAACTTTCAAAAGCGGGAAACTTGTTTGATTATGATCTAG | CDS |
| | GATTCTAGGTCTTTTTTTTTAAGTTCGAGACAGAAAACCAAAAAAAAACA | In plasmid |
| | GCTAATAAGAAGAAGAAAAACAATACAGCAATATTCCCACAAAAATAAAT | in SEQ ID |
| | TTGTTCTTTTGCGTACTAGCCATAGGATTTAATTATATATATGGGCCAGT | NO: 60 |
| | CCAGAAGCATTGTCACTGATAGATCATAGACTAGTAAAAAAGAATGAACC | and SEQ |
| | ACTTCAGAACACTAACTATATATATAAAGATAGATTCATATCTATTTAGA | ID NO: |
| | TCGTGATTATTAATACAAGAAAATATTTGTATTTCTTAATTTTTTGTTTT | 101. |
| | TAAATTTATGTAAATATATTGACATCTTTTTTAACGAAAATTCAAATTTT | These are |
| | AATTCATTATATTACAAGAAATTAACACATTTTACTAGACCAACTCTCAT | likely the |
| | GTTACAACACTATATATGTTGAACATTACAAGAATGCCACTTTATCTCAC | same |
| | CCCTTTAATTAGGAAAAGCAAAAATCTATTTGCATATAATTTTGTACCAA | plasmid. |
| | AAAATTATACAATAAAAAAGAAAAAGAGAAAAGTAAAAGATAAGACGACT | |
| | GATGTAATAAAAAGAGAGAAAAAATAATACTAAAAAATAATATAAATAAC | |
| | ATAACTCTTAAAAAAAAAAGGTAACACTGTTGAGTATTTTTTTTATTACT | |
| | TTTCTCTAAAAAGGTATCATGGTTTCACTAATGTCCAGGTTTTTTTTTTC | |
| | CAAATAAACTCCTTTCCGGTGGTAGGTATCGTTTCACACTTGAATACTTT | |
| | TTTGCAATATTTTTCGTAACACTTTATTTCTCTCCATTACATAATTTTAC | |
| | CCTTGCCTATGTCTTTATTCTGCCACATATACCCTAAGAGTGAGCAAATG | |
| | TGTGCACGCATATAACCTGATTGGACTATATATCGGACTTGATACAAATG | |
| | TGTGCTCACATGAAGAGAAAAAGGATTGAACCCATGATCATCATCATGTC | |
| | ACATGCAGATGCCATGCATTCAACACCAAAGTTATGATCAGAAAACCTCT | |
| | CAAGGTAGGTGACTCACCAAATGAACAAGCCAATTTTTTGCTTCCCAAAT | |
| | TTCACCATAAATAGGGACACTACTCCATCCAAACCATACACGACCACATA | |
| | CAGTGGGATTAGTAAGTAAACAAATTAATTAATAACCAAAAGTTATAATT | |
| | TCAATTGAATTTAGTCATGAAAAAAGCTGTAATAAACGGTGAACAAATAC | |
| | GCAGCATATCTGACCTCCACCAGACTCTCAAGAAAGAGCTTGCTTTGCCC | |
| | GAGTATTATGGTGAGAATCTGGACGCATTGTGGGACTGCCTCACAGGATG | |
| | GGTGGAGTACCCATTGGTCTTGGAGTGGCGTCAGTTTGAACAGAGCAAAC | |
| | AGTTGACTGAGAACGGTGCCGAGTCCGTTCTTCAAGTATTTCGCGAGGCC | |
| | AAGGCCGAAGGCTGCGACATAACCATCATCCTCTCT | |
| 44 | TTTGTAATATTATGATTTACGTAAAAATCTATCTAATGATTTTATTTAAA | Barstar |
| | CTATAGTTTTGAACTTTTCCAACTGAAATTTCAACACAGTAATTATGGTT | construct |
| | GCCTGTAACTCATATTTTGCAAGCCCCACTTCAATTATGCAGCCTAACCC | driven by |
| | AAAAGTGGGGATATGTGATTTCAAAGGATATATGCATCAATCATTTCTCC | TA29b |
| | AAATTAGGAAAGTAATTTATATAAAATTAACTTGACTTTAAGTTAGAACT | promoter. |
| | GCATTTTACTGAATAAAAGTTATAATTATTAATAAATAAATGGTTAAGAT | In plasmid |
| | TTTAACGTTATAATATATTCATACAGTACAAATTCAAGTTTTTATTCTTG | in SEQ ID |
| | AAATAAAATTTTTATTTTAGGTATTTCATTTTTATGCAGTCATGTGTCTT | NO: 61 |
| | TTTTTTATTTTTGAAACATATTTTATGGTCATTTATATCAATTCAAATTA | and SEQ |
| | GAATTTTCTTCCTCTCTAACTAATTATTCAACAACAAAAAAAAACTATTT | ID NO: |
| | TTTCAAGTTTTTATTTTACTCAGTGTTTTAATCAATATTAAGCATAATGA | 100. |
| | GTATTTATAAATCATTCTCAAAATAATATTTTACATGTTAAATAATTTTT | These are |
| | AAGTATTTTTAAGAGTGTGTTTAATTGTGTTTTAATTTCTTAGTCTATTT | likely the |
| | TTTAAAAACTATTTTATGAAACATTTTTCATAAACTGATGTTTAGAGAGT | same |
| | AAAAGTGGAAAAGTAAGATGTGAAAATACAAGGAAGAACATGAATAATAG | plasmid. |
| | AAAATTAAAAGTAAAAAACCCAAACATCGATCATAGAGATGTTATAGTTT | |
| | TTTGGTTTTGAAAAAACTAAAAAAAGAGATAGTAGTTTTAAAAACAGTTT | |
| | TCGAAACTATTTTTATCAAACAACATAATTTTTTTAGCTTTTAAATAAAA | |
| | ACAGCTTTAAAAAATGTCCAGGTTTTTTTCCAAATAAATTCAATTCCATA | |
| | TTTTAATAAGTAATGGTTAATCCAGTGGTAGGTATATATTGTTTAACACT | |
| | TGAAATTTTTTTCCATTCAATTTTTTTAATATTTTTCATAACACTTAATT | |
| | TATTTATTACATATATCATATGTTATATTATTATTTTTCTCTATTCTCTT | |
| | CCTTTCTCTCTAGTTATTTGTAAAAAAAAAAACATATAATTTATTTCCAT | |
| | TGCATAAATTTTATCCATGTCTATCCATGCCTATGTTTTTATTCTGCCAC | |
| | ATATACCCTAAGTGTTAGCAAATATGTGCACACATTAACCTAATTAAACT | |
| | ATACTAGAACTATATATCGGACTTGATAGAAAGTGGAGATAAGCCAAGGA | |
| | AGACAAGAAAAAGGATTGAACACATGATCATCCTCATGTCACATGCAGTT | |
| | GCCATGGATCCAACACCAAAGTTATGATCAGAAAACCTCTCAAGGCAGGT | |
| | GACTCACGAAATGAACAAGCCAATTTGCTTCCAAAATTCACCATAAATAG | |
| | GGACACTACTCAATGCAAACCATGCATACACAATCATATACAGTGGGATT | |
| | AGTAAACAAATTAATTAATAACCAAAAGTTATTTAAATTGAAGTCATGAA | |
| | AAAAGCTGTAATAAACGGTGAACAAATACGCAGCATATCTGACCTCCACC | |
| | AGACTCTCAAGAAAGAGCTTGCTTTGCCCGAGTATTATGGTGAGAATCTG | |
| | GACGCATTGTGGGACTGCCTCACAGGATGGGTGGAGTACCCATTGGTCTT | |
| | GGAGTGGCGTCAGTTTGAACAGAGCAAACAGTTGACTGAGAACGGTGCCG | |
| | AGTCCGTTCTTCAAGTATTTCGCGAGGCCAAGGCCGAAGGCTGCGACATA | |
| | ACCATCATCCTCTCT | |
| 45 | aagcgcccagccgctaccaaaaaagctggacaggctaaaaaaaagaaggg | Tail-SRDX |
| | tgacggcagcgggtccggctccgggtccggttccgtcgatctcgatttgg | CT |
| | atctggaacttcgcctggggttcgcactcgatctcgaccttgaactgcgt | (Nucleoplasmin |
| | cttggctttgctctcgatcttgatctggagttgaggcttggattcgctta | NLS |
| | g | + linker + |
| | | 3x SRDX) |
| | | WITHOUT |
| | | Cas9 |
| 46 | KRPAATKKAGQAKKKKGDGSGSGSGSGSVDLDLDLEELRLGFALDLDLEL | Engineered SRDX |
| | RLGFALDLDLELRLGFA | Protein |
| | | WITHOUT |
| | | Cas9 |
| 47 | aagaggcctgccgccacaaagaaagccggccaggcaaagaaaaaaaaacc | Tail-TV |
| | caggggagggagtggtggactcctcgaccctggcactccaatggacgccg | CT |
| | atctcgtagcatcatctaccgtcgtgtgggagcaggacgccgaccctttt | WITHOUT |
| | gctggcacagctgatgatttcccagcttttaatgaggaagaattggcttg | Cas9 |
| | gctgatggagcttctcccacaaggtggtagcggcgggctcctcgatcccg | |
| | gtactcccatggacgcagatcttgttgctagctccactgttgtatgggag | |
| | caggacgccgatcccttcgctggaaccgccgatgatttcccagcattcaa | |
| | cgaggaagaacttgcatggctcatggagctcttgccccaagctcgcggtg | |
| | gatctgggggtttgttggatcccggaaccccaatggacgcagacctcgtg | |
| | gcttctagtaccgtagtatgggaacaggacgctgaccctttcgccggcac | |
| | cgcagatgacttcccagcattcaacgaggaggaactggcctggctgatgg | |
| | aacttctgcctcagggtggatccggaggcctcttggaccccggtacccct | |
| | atggacgctgacctggtcgcatcttctaccgtggtttgggagcaagatgc | |
| | cgacccattcgcaggaacagctgatgacttccccgcattcaatgaagaag | |
| | agctcgcatggctgatggaattgcttccacaagccagaggcggctctgga | |
| | gggcttcttgatcccggaacacccatggatgccgacctcgtggcctctag | |
| | caccgtggtatgggaacaagacgcagatccattcgcagggaccgcagacg | |
| | atttccccgcttttaacgaggaggagcttgcttggctcatggagctgctg | |
| | cctcagggtggatccgggggacttctcgacccaggcacacccatggatgc | |
| | cgatctcgtcgccagttctaccgttgtctgggagcaagacgcagaccctt | |
| | tcgccggaactgcagacgacttccctgcctttaatgaagaagaactggct | |
| | tggctcatggaactgctcccccaggctagagggggtagcggcgggggcgg | |
| | cagcggaggtgatgcacttgacgacttcgatcttgatatgcttggcagcg | |
| | acgcacttgatgacttcgatttggatatgctggggtccgatgctcttgac | |
| | gattttgatttggacatgttgggttcagacgcactcgatgatttcgattt | |
| | ggacatgctggcacgtggaagcgatgcccttgatgactttgacctggata | |
| | tgctcgggtccgatgctttggacgacttcgacctcgacatgttgggaagt | |
| | gatgctcttgatgatttcgatcttgatatgcttgggtccgacgctctcga | |
| | tgattttgacctcgatatgctctga | |
| 48 | KRPAATKKAGQAKKKKPRGGSGGLLDPGTPMDADLVASSTVVWEQDADPF | Tail - TV |
| | AGTADDFPAFNEEELAWLMELLPQGGSGGLLDPGTPMDADLVASSTVVWE | CT |
| | QDADPFAGTADDFPAFNEEELAWLMELLPQARGGSGGLLDPGTPMDADLV | Engineered |
| | ASSTVVWEQDADPFAGTADDFPAFNEEELAWLMELLPQGGSGGLLDPGTP | protein |
| | MDADLVASSTVVWEQDADPFAGTADDFPAFNEEELAWLMELLPQARGGSG | WITHOUT |
| | GLLDPGTPMDADLVASSTVVWEQDADPFAGTADDFPAFNEEELAWLMELL | Cas9 |
| | PQGGSGGLLDPGTPMDADLVASSTVVWEQDADPFAGTADDFPAFNEEELA | |
| | WLMELLPQARGGSGGGGSGGDALDDFDLDMLGSDALDDFDLDMLGSDALD | |
| | DFDLDMLGSDALDDFDLDMLARGSDALDDFDLDMLGSDALDDFDLDMLGS | |
| | DALDDFDLDMLGSDALDDFDLDML | |
| 49 | atggtgtccaagggcgaagaggtgattaaagagttcatgaggttcaaagt | Td_Tomato |
| | gagaatggagggcagtatgaacggccatgagttcgagatcgaaggtgaag | Fluorotag |
| | gcgaaggtagaccatacgagggcacccagacggcgaaactgaaagtgacg | CDS |
| | aagggcggcccactgcccttcgcctgggacattctgagtccacaatttat | |
| | gtacggttccaaggcatacgttaagcaccccgccgacatccctgattaca | |
| | aaaagctgtcgttcccggaggggtttaaatgggaaagagtgatgaatttt | |
| | gaggacggagggctcgtcactgtgactcaagactectcacttcaagacgg | |
| | caccctaatttacaaggtgaagatgaggggaacaaatttcccccctgatg | |
| | gccctgttatgcagaaaaaaacaatgggatgggaggcatccaccgagaga | |
| | ctttacccgcgcgacggagtgctcaaaggcgaaatccatcaggcactgaa | |
| | actaaaggacggcggccactacctggttgagttcaaaactatatatatgg | |
| | ccaagaaacccgtccagttgccgggatattactatgtggacacaaaactg | |
| | gatataacgtcccacaacgaagattacaccatagtggaacagtatgagcg | |
| | cagtgagggtcgtcaccatctattcctcggacatggcacgggttccacgg | |
| | gctcgggttccagcggcacggcaagctcggaggacaacaacatggcggtg | |
| | atcaaagagttcatgcggtttaaagtccgtatggagggcagcatgaatgg | |
| | gcacgaatttgaaattgagggagaaggcgagggccgaccatatgagggga | |
| | cacaaaccgcgaaactaaaagtcaccaaaggtgggccgcttccttttgcc | |
| | tgggacattctctctcctcagttcatgtatggctccaaggcctacgttaa | |
| | acatccggctgatatccccgattataagaaattgtctttcccggaggggt | |
| | ttaaatgggagcgggttatgaatttcgaggatggtggactggttactgtt | |
| | acacaagattcgagtctgcaggatggcacattgatatacaaagtcaaaat | |
| | gaggggtacgaattttcccccggacggcccggtgatgcagaaaaaaacta | |
| | tgggctgggaggccagcacggagaggctgtacccgagggacggggttttg | |
| | aaaggggagatacaccaggcgcttaaactgaaggacggggggcactatct | |
| | ggtggaatttaagacgatatatatggcgaaaaagcccgtgcagctacctg | |
| | gctactactacgtcgataccaaactcgatattacctctcataacgaggac | |
| | tacactatcgtggagcaatatgaaagatcagaaggacgtcaccacctttt | |
| | tctatacggaatggacgagctttacaaa | |
| 50 | MVSKGEEVIKEFMRFKVRMEGSMNGHEFEIEGEGEGRPYEGTQTAKLKVT | Engineered |
| | KGGPLPFAWDILSPQFMYGSKAYVKHPADIPDYKKLSFPEGFKWERVMNF | td_Tomato |
| | EDGGLVTVTQDSSLQDGTLIYKVKMRGTNFPPDGPVMQKKTMGWEASTER | Fluorrotag |
| | LYPRDGVLKGEIHQALKLKDGGHYLVEFKTIYMAKKPVQLPGYYYVDTKL | Protein |
| | DITSHNEDYTIVEQYERSEGRHHLFLGHGTGSTGSGSSGTASSEDNNMAV | |
| | IKEFMRFKVRMEGSMNGHEFEIEGEGEGRPYEGTQTAKLKVTKGGPLPFA | |
| | WDILSPQFMYGSKAYVKHPADIPDYKKLSFPEGFKWERVMNFEDGGLVTV | |
| | TQDSSLQDGTLIYKVKMRGTNFPPDGPVMQKKTMGWEASTERLYPRDGVL | |
| | KGEIHQALKLKDGGHYLVEFKTIYMAKKPVQLPGYYYVDTKLDITSHNED | |
| | YTIVEQYERSEGRHHLFLYGMDELYK | |
| 51 | atggctcagtccaagcacggccttaccaaggagatgaccatgaagtacag | ZsGreen |
| | gatggagggctgcgtggatggccacaagttcgtgatcaccggcgagggca | Fluorotag |
| | tcggctacccattcaagggcaaacaggctatcaacctttgcgtggtggag | CDS |
| | ggcggcccacttccattcgctgaggatatcctttccgctgctttcatgta | |
| | cggcaacagggtgttcaccgagtacccacaggatatcgctgattacttca | |
| | agaactcctgcccagctggctacacctgggataggtccttccttttcgag | |
| | gatggcgctgtgtgcatctgcaacgctgatatcaccgtgtccgtggagga | |
| | gaactgcatgtaccacgagtccaagttctacggcgtgaacttcccagctg | |
| | atggcccagtgatgaagaagatgaccgataactgggagccatcctgcgag | |
| | aagatcatcccagtgccaaagcagggcatccttaagggcgatgtgtccat | |
| | gtaccttcttcttaaggatggcggcaggcttaggtgccagttcgataccg | |
| | tgtacaaggctaagtccgtgccaaggaagatgccagattggcacttcatc | |
| | cagcacaagcttaccagggaggataggtccgatgctaagaaccagaagtg | |
| | gcaccttaccgagcacgctatcgcttccggctccgcccttcca | |
| 52 | MAQSKHGLTKEMTMKYRMEGCVDGHKFVITGEGIGYPFKGKQAINLCVVE | Engineered |
| | GGPLPFAEDILSAAFMYGNRVFTEYPQDIADYFKNSCPAGYTWDRSFLFE | ZsGreen |
| | DGAVCICNADITVSVEENCMYHESKFYGVNFPADGPVMKKMTDNWEPSCE | Fluorotag |
| | KIIPVPKQGILKGDVSMYLLLKDGGRLRCQFDTVYKAKSVPRKMPDWHFI | Protein |
| | QHKLTREDRSDAKNQKWHLTEHAIASGSALP | |
| 53 | atgggtcagtcccttatgttacgtcctgtagaaaccccaacccgtgaaat | GUS DNA |
| | caaaaaactcgacggcctgtgggcattcagtctggatcgcgaaaactgtg | Sequence |
| | gaattgatcagcgttggtgggaaagcgcgttacaagaaagccgggcaatt | (Includes |
| | gctgtgccaggcagttttaacgatcagttcgccgatgcagatattcgtaa | Introns) |
| | ttatgcgggcaacgtctggtatcagcgcgaagtctttataccgaaaggta | |
| | agtcttactctctcttttttggtctgtatttttaattttttgaagtatac | |
| | tatttgtactgacgctaataatcttttttcaggttgggcaggccagcgta | |
| | tcgtgctgcgtttcgatgcggtcactcattacggcaaagtgtgggtcaat | |
| | aatcaggaagtgatggagcatcagggcggctatacgccatttgaagccga | |
| | tgtcacgccgtatgttattgccgggaaaagtgtacgtatcaccgtttgtg | |
| | tgaacaacgaactgaactggcagactatcccgccgggaatggtgattacc | |
| | gacgaaaacggcaagaaaaagcagtcttacttccatgatttctttaacta | |
| | tgccggaatccatcgcagcgtaatgctctacaccacgccgaacacctggg | |
| | tggacgatatcaccgtggtgacgcatgtcgcgcaagactgtaaccacgcg | |
| | tctgttgactggcaggtacttcatgcttcaacgtgtaacttaagagatac | |
| | tgtgtgaaattttatatttccatacatttgcttgacctttgctttttgtc | |
| | aatttttttccccttacaggtggtggccaatggtgatgtcagcgttgaac | |
| | tgcgtgatgcggatcaacaggtggttgcaactggacaaggcactagcggg | |
| | actttgcaagtggtgaatccgcacctctggcaaccgggtgaaggttatct | |
| | ctatgaactgtgcgtcacagccaaaagccagacagagtgtgatatctacc | |
| | cgcttcgcgtcggcatccggtcagtggcagtgaagggcgaacagttcctg | |
| | attaaccacaaaccgttctactttactggctttggtcgtcatgaagatgc | |
| | ggacttgcgtggcaaaggattcgataacgtgctgatggtgcacgaccacg | |
| | cattaatggactggattggggccaactcctaccgtacctcgcattaccct | |
| | tacgctgaagagatgctcgactgggcagatgaacatggcatcgtggtgat | |
| | tgatgaaactgctgctgtcggctttaacctctctttaggcattggtttcg | |
| | aagcgggcaacaagccgaaagaactgtacagcgaagaggcagtcaacggg | |
| | gaaactcagcaagcgcacttacaggcgattaaagagctgatagcgcgtga | |
| | caaaaaccacccaagcgtggtgatgtggagtattgccaacgaaccggata | |
| | cccgtccgcaaggtgcacgggaatatttcgcgccactggcggaagcaacg | |
| | cgtaaactcgacccgacgcgtccgatcacctgcgtcaatgtaatgttctg | |
| | cgacgctcacaccgataccatcagcgatctctttgatgtgctgtgcctga | |
| | accgttattacggatggtatgtccaaagcggcgatttggaaacggcagag | |
| | aaggtactggaaaaagaacttctggcctggcaggagaaactgcatcagcc | |
| | gattatcatcaccgaatacggcgtggatacgttagccgggctgcactcaa | |
| | tgtacaccgacatgtggagtgaagagtatcagtgtgcatggctggatatg | |
| | tatcaccgcgtctttgatcgcgtcagcgccgtcgtcggtgaacaggtatg | |
| | gaatttcgccgattttgcgacctcgcaaggcatattgcgcgttggcggta | |
| | acaagaaagggatcttcactcgcgaccgcaaaccgaagtcggcggctttt | |
| | ctgctgcaaaaacgctggactggcatgaacttcggtgaaaaaccgcagca | |
| | gggaggcaaacaagg | |
| 54 | atgggtcagtcccttatgttacgtcctgtagaaaccccaacccgtgaaat | GUS CDS |
| | caaaaaactcgacggcctgtgggcattcagtctggatcgcgaaaactgtg | |
| | gaattgatcagcgttggtgggaaagcgcgttacaagaaagccgggcaatt | |
| | gctgtgccaggcagttttaacgatcagttcgccgatgcagatattcgtaa | |
| | ttatgcgggcaacgtctggtatcagcgcgaagtctttataccgaaaggtt | |
| | gggcaggccagcgtatcgtgctgcgtttcgatgcggtcactcattacggc | |
| | aaagtgtgggtcaataatcaggaagtgatggagcatcagggcggctatac | |
| | gccatttgaagccgatgtcacgccgtatgttattgccgggaaaagtgtac | |
| | gtatcaccgtttgtgtgaacaacgaactgaactggcagactatcccgccg | |
| | ggaatggtgattaccgacgaaaacggcaagaaaaagcagtcttacttcca | |
| | tgatttctttaactatgccggaatccatcgcagegtaatgctctacacca | |
| | cgccgaacacctgggtggacgatatcaccgtggtgacgcatgtcgcgcaa | |
| | gactgtaaccacgcgtctgttgactggcaggtggtggccaatggtgatgt | |
| | cagcgttgaactgcgtgatgcggatcaacaggtggttgcaactggacaag | |
| | gcactagcgggactttgcaagtggtgaatccgcacctctggcaaccgggt | |
| | gaaggttatctctatgaactgtgcgtcacagccaaaagccagacagagtg | |
| | tgatatctacccgcttcgcgtcggcatccggtcagtggcagtgaagggcg | |
| | aacagttcctgattaaccacaaaccgttctactttactggctttggtcgt | |
| | catgaagatgcggacttgcgtggcaaaggattcgataacgtgctgatggt | |
| | gcacgaccacgcattaatggactggattggggccaactcctaccgtacct | |
| | cgcattacccttacgctgaagagatgctcgactgggcagatgaacatggc | |
| | atcgtggtgattgatgaaactgctgctgtcggctttaacctctctttagg | |
| | cattggtttcgaagcgggcaacaagccgaaagaactgtacagcgaagagg | |
| | cagtcaacggggaaactcagcaagcgcacttacaggcgattaaagagctg | |
| | atagcgcgtgacaaaaaccacccaagcgtggtgatgtggagtattgccaa | |
| | cgaaccggatacccgtccgcaaggtgcacgggaatatttcgcgccactgg | |
| | cggaagcaacgcgtaaactegacccgacgcgtccgatcacctgcgtcaat | |
| | gtaatgttctgcgacgctcacaccgataccatcagcgatctctttgatgt | |
| | gctgtgcctgaaccgttattacggatggtatgtccaaagcggcgatttgg | |
| | aaacggcagagaaggtactggaaaaagaacttctggcctggcaggagaaa | |
| | ctgcatcagccgattatcatcaccgaatacggcgtggatacgttagccgg | |
| | gctgcactcaatgtacaccgacatgtggagtgaagagtatcagtgtgcat | |
| | ggctggatatgtatcaccgcgtctttgatcgcgtcagcgccgtcgtcggt | |
| | gaacaggtatggaatttcgccgattttgcgacctcgcaaggcatattgcg | |
| | cgttggcggtaacaagaaagggatcttcactcgcgaccgcaaaccgaagt | |
| | cggcggcttttctgctgcaaaaacgctggactggcatgaacttcggtgaa | |
| | aaaccgcagcagggaggcaaacaagg | |
| 55 | MGQSLMLRPVETPTREIKKLDGLWAFSLDRENCGIDQRWWESALQESRAI | Engineered Gus |
| | AVPGSFNDQFADADIRNYAGNVWYQREVFIPKGWAGQRIVLRFDAVTHYG | Protein |
| | KVWVNNQEVMEHQGGYTPFEADVTPYVIAGKSVRITVCVNNELNWQTIPP | |
| | GMVITDENGKKKQSYFHDFFNYAGIHRSVMLYTTPNTWVDDITVVTHVAQ | |
| | DCNHASVDWQVVANGDVSVELRDADQQVVATGQGTSGTLQVVNPHLWQPG | |
| | EGYLYELCVTAKSQTECDIYPLRVGIRSVAVKGEQFLINHKPFYFTGFGR | |
| | HEDADLRGKGFDNVLMVHDHALMDWIGANSYRTSHYPYAEEMLDWADEHG | |
| | IVVIDETAAVGFNLSLGIGFEAGNKPKELYSEEAVNGETQQAHLQAIKEL | |
| | IARDKNHPSVVMWSIANEPDTRPQGAREYFAPLAEATRKLDPTRPITCVN | |
| | VMFCDAHTDTISDLFDVLCLNRYYGWYVQSGDLETAEKVLEKELLAWQEK | |
| | LHQPIIITEYGVDTLAGLHSMYTDMWSEEYQCAWLDMYHRVFDRVSAVVG | |
| | EQVWNFADFATSQGILRVGGNKKGIFTRDRKPKSAAFLLQKRWTGMNFGE | |
| | KPQQGGKQ | |
| 56 | ccaaagaagaagcggaaggtctaa | SV40 NLS |
| 57 | PKKKRKV | Engineered NLS |
| | | Protein |
| 58 | gtgccgaattcggatccagcgatctggattttagtactggattttggttt | pTA29a/b- |
| | taggaattagaaattttattgatagaagtattttacaaatacaaatacat | Control |
| | actaagggtttcttatatgctcaacacatgagcgaaaccctataggaacc | Likely |
| | ctaattcccttatctgggaactactcacacattattatggagaaactcga | same as |
| | gcttgtcgatcgactctagctagagaagctcactttccaacaaccttagt | SEQ ID |
| | aatttctcccttaacataatgaacaaattcttcaagttgatcagctctag | NO: 109 |
| | aagcaagtctatcttcttcttgtccaagataagcttgtctagcttcaaga | |
| | ataactggttgatattgagctggaagtctttccatagcccaatcagcagc | |
| | aacatcctttggagcaatctttccagtaacagcagaataccaaattctag | |
| | aaagagtaagaacaacatttctttcatctccagcccaatctggtggagaa | |
| | ttccaaagagtaagagtttcattaagagcttcaaaaagatcttgttctgg | |
| | aactggatcaaaaagttcttcagcagctggtccaacaagagcaacagaat | |
| | gttctctagccttagtaagaagaatagcaagatcaatatcaatagtagct | |
| | ggttcaaaaattccagcaagaatatcatttctttgccattctccaaattg | |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattitggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcagcgtcgatctag | |
| | taacatagatgacaccgcgcgcgataatttatcctagtttgcgcgctata | |
| | ttttgttttctatcgcgtattaaatgtataattgcgggactctaatcata | |
| | aaaacccatctcataaataacgtcatgcattacatgttaattattacatg | |
| | cttaacgtaattcaacagaaattatatgataatcatcgcaagaccggcaa | |
| | caggattcaatcttaagaaactttattgccaaatgtttgaacgatctgct | |
| | tgacaagcttagaccttccgcttcttctttggcgaacttggaagggcgga | |
| | gccggaagcgatagcgtgctcggtaaggtgccacttctggttcttagcat | |
| | cggacctatcctccctggtaagcttgtgctggatgaagtgccaatctggc | |
| | atcttccttggcacggacttagccttgtacacggtatcgaactggcacct | |
| | aagcctgccgccatccttaagaagaaggtacatggacacatcgcccttaa | |
| | ggatgccctgctttggcactgggatgatcttctcgcaggatggctcccag | |
| | ttatcggtcatcttcttcatcactgggccatcagctgggaagttcacgcc | |
| | gtagaacttggactcgtggtacatgcagttctcctccacggacacggtga | |
| | tatcagcgttgcagatgcacacagcgccatcctcgaaaaggaaggaccta | |
| | tcccaggtgtagccagctgggcaggagttcttgaagtaatcagcgatatc | |
| | ctgtgggtactcggtgaacaccctgttgccgtacatgaaagcagcggaaa | |
| | ggatatcctcagcgaatggaagtgggccgccctccaccacgcaaaggttg | |
| | atagcctgtttgcccttgaatgggtagccgatgccctcgccggtgatcac | |
| | gaacttgtggccatccacgcagccctccatcctgtacttcatggtcatct | |
| | ccttggtaaggccgtgcttggactgagccattgacttcaatttaaataac | |
| | ttttggttattaattaatttgtttactaatcccactgtatatgattgtgt | |
| | atgcatggtttgcattgagtagtgtccctatttatggtgaattttggaag | |
| | caaattggcttgttcatttcgtgagtcacctgccttgagaggttttctga | |
| | tcataactttggtgttggatccatggcaactgcatgtgacatgaggatga | |
| | tcatgtgttcaatcctttttcttgtctaccttggcttatctccactttct | |
| | atcaagtccgatatatagttctagtatagtttaattaggttaatgtgtgc | |
| | acatatttgctaacacttagggtatatgtggcagaataaaaacataggca | |
| | tggatagacatggataaaatttatgcaatggaaataaattatatgttttt | |
| | ttttttacaaataactagagagaaaggaagagaatagagaaaaataataa | |
| | tataacatatgatatatgtaataaataaattaagtgttatgaaaaatatt | |
| | aaaaaaattgaatggaaaaaaatttcaagtgttaaacaatatatacctac | |
| | cactggattaaccattacttattaaaatatggaattgaatttatttggaa | |
| | aaaaacctggacattttttaaagctgtttttatttaaaagctaaaaaaat | |
| | tatgttgtttgataaaaatagtttcgaaaactgtttttaaaactactatc | |
| | tctttttttagttttttcaaaaccaaaaaactataacatctctatgatcg | |
| | atgtttgggttttttacttttaattttctattattcatgttcttccttgt | |
| | attttcacatcttacttttccacttttactctctaaacatcagtttatga | |
| | aaaatgtttcataaaatagtttttaaaaaatagactaagaaattaaaaca | |
| | caattaaacacactcttaaaaatacttaaaaattatttaacatgtaaaat | |
| | attattttgagaatgatttataaatactcattatgcttaatattgattaa | |
| | aacactgagtaaaataaaaacttgaaaaaatagtttttttttgttgttga | |
| | ataattagttagagaggaagaaaattctaatttgaattgatataaatgac | |
| | cataaaatatgtttcaaaaaaaaaaaaagacacatgactgcataaaaatg | |
| | aaatacctaaaataaaaattttatttcaagaataaaaacttgaatttgta | |
| | ctgtatgaatatattataacgttaaaatcttaaccatttatttattaata | |
| | attataacttttattcagtaaaatgcagttctaacttaaagtcaagttaa | |
| | ttttatataaattactttcctaatttggagaaatgattgatgcatatatc | |
| | ctttgaaatcacatatccccacttttgggttaggctgcataattgaagtg | |
| | gggcttgcaaaatatgagttacaggcaaccataattactgtgttgaaatt | |
| | tcagttggaaaagttcaaaactatagtttaaataaaatcattagatagat | |
| | ttttacgtaaatcataatattacaaactccttacgaattcccatggagcg | |
| | tccctcggtcacatgtgcatcctctagtggagaagttgtaatgagttgct | |
| | ggcctctcttgggattgttcatcaaagtagctgtccctacacaccacaac | |
| | agaatggtgttgtggagagaaagcacagacacatccttgagatggcaagg | |
| | gcacttaagtttcagagtggtgtacctaccaggttttggggagactgtgt | |
| | caagactgttgtatacctgatcaatagactccctactccaatcttacaag | |
| | gcaaatgtccatatgaactactctatcagaaacatgccaagcttgatcac | |
| | ttgagagtgtttgggtgtcttgcttttgcagcaacactgcccaaaggtga | |
| | caaacttgcacctagagccaagcgaaccatcttcattggttattctgaga | |
| | ctcaaaagggttacaggttgtatgacttggataataaggtgatcatagtg | |
| | agcagggatgtagttttcagagagtttcagttccctttcaaagagtccat | |
| | ctcccatgaacctgatatgttcactcgagcaggttcacttagctctgaag | |
| | atacagctttgcagctagtgtataatgatatttttccttccagctttcat | |
| | actgatgatgatgatactccagttcttattcctgacattgtagaggccaa | |
| | tgaggatatcactttggggactcattctacccaaaatcatatcacttcag | |
| | ctgatgctgcaccttcaggccacctgcatgctcctgcagagtcagttgag | |
| | tcagctgagcctgacctagcaaatgctgaatttgagcaccatactgctgt | |
| | agctgatccctctctagttccagcaaacctgcacactagacctaaacgca | |
| | atgcaggtcctcccatctggctcaaggactttgtgacactgaacaaaggc | |
| | tctagggatgttccataccctatagccaactatgtttcctatgatcactt | |
| | gcctgttcactatcaagcttatttgagtgctttctccacttatactgaac | |
| | ctaaatccttcaaagaagcagctcaagatgagaaatggatggaggccatg | |
| | tcccttgagatacaggctcttgaggataataacacctgggagattgtccc | |
| | tttaccccctggtaaacagcctatagggtccaaatgggtgtacaaaatta | |
| | aatacaaagctaatggtgaagttgacaggtttaaggcaaggctagtggcc | |
| | aagggatacactcagcaagaaggccttgactaccatgaaactttttctcc | |
| | agtggccaaaatggtcactgtaaaagctgtcatatctgttgctgcttcca | |
| | agggctggttccttttccagatggatgtcaacaatgcttttctacaaggt | |
| | gacctcatggaagaagtttatatgtctctgcctcagggttttcacagcca | |
| | aggggaaaccaaagtgtgcaggctgttgaatccctctatggtttaaagca | |
| | agcatcaaggcagtggaacatcaagcttaccactgtccttatgcaggctg | |
| | gttttatgcaaagtgcttatgatcactccttgtttaccaaaagagagggg | |
| | actgaccttgtcataatcctgatttatgtggatgatctgttaataacagg | |
| | cagcagcaatgttttgatttcagaagcaaaggcaaccctgcatcagcatt | |
| | tcaagatgaaagatttgggagaactaaaatacttcttaggcattgaggtg | |
| | ctgagatcagaaaagggaatcctactgaaccagaggaagtatgcactgga | |
| | actgatatcaggtgttggtctgggaggctgcagaccagtgtcaaccccaa | |
| | tggaacagaaccaaaggctgactactgtagaatatgacaagcacttagga | |
| | aagactgatgatgcagagttagaggatgtgggatcctatcagagactggt | |
| | gggaaagcttctctacttgacaatcacaaggccagatatcagctttgcag | |
| | tgcaggtgctgagtcaattcatgcagcaacccaaacagtcacacctggaa | |
| | gcagcattgagggttgtgaagtacataaaaggttctccaggtgttaggaa | |
| | cgctaagcttgaattcttgcggaggtttccatagcctaaggccacgagtg | |
| | cacgtgttcataggctagttgtgttaaccttggggagcagtcgacaattt | |
| | ggattgcatcgaggtcttgccaaattttactttcaagacagtgatttcga | |
| | cggcacaataaacttccaattagttatcttctcttttgttctgtattgtg | |
| | tttttttctatttttctaacaatggaaatagtatataccggccaacagtg | |
| | ttgcactgttgtctggaccttgtcaaaggttcgacagtgcatagtttgga | |
| | ctcgatgtggcatatgggggttctgataagtgatgagattatgaaaataa | |
| | tttaattttagctattaaaaatacaatacaactttcaaaagcgggaaact | |
| | tgtttgattatgatctaggattctaggtctttttttttaagttcgagaca | |
| | gaaaaccaaaaacaaaacagctaataagaagaagaaaaacaatacagcaa | |
| | tattcccacaaaaataaatttgttcttttgcgtactagccataggattta | |
| | attatatatatgggccagtccagaagcattgtcactgatagatcatagac | |
| | tagtaaaaaagaatgaaccacttcagaacactaactatatatataaagat | |
| | agattcatatctatttagatcgtgattattaatacaagaaaatatttgta | |
| | tttcttaattttttgtttttaaatttatgtaaatatattgacatcttttt | |
| | taacgaaaattcaaattttaattcattatattacaagaaattaacacatt | |
| | ttactagaccaactctcatgttacaacactatatatgttgaacattacaa | |
| | gaatgccactttatctcacccctttaattaggaaaagcaaaaatctattt | |
| | gcatataattttgtaccaaaaaattatacaataaaaaagaaaaagagaaa | |
| | agtaaaagataagacgactgatgtaataaaaagagagaaaaaataatact | |
| | aaaaaataatataaataacataactcttaaaaaaaaaaggtaacactgtt | |
| | gagtattttttttattacttttctctaaaaaggtatcatggtttcactaa | |
| | tgtccaggttttttttttccaaataaactectttccggtggtaggtatcg | |
| | tttcacacttgaatacttttttgcaatatttttcgtaacactttatttct | |
| | ctccattacataattttacccttgcctatgtctttattctgccacatata | |
| | ccctaagagtgagcaaatgtgtgcacgcatataacctgattggactatat | |
| | atcggacttgatacaaatgtgtgctcacatgaagagaaaaaggattgaac | |
| | ccatgatcatcatcatgtcacatgcagatgccatgcattcaacaccaaag | |
| | ttatgatcagaaaacctctcaaggtaggtgactcaccaaatgaacaagcc | |
| | aattttttgcttcccaaatttcaccataaatagggacactactccatcca | |
| | aaccatacacgaccacatacagtgggattagtaagtaaacaaattaatta | |
| | ataaccaaaagttataatttcaattgaatttagtcaatggtgtccaaggg | |
| | cgaagaggtgattaaagagttcatgaggttcaaagtgagaatggagggca | |
| | gtatgaacggccatgagttcgagatcgaaggtgaaggcgaaggtagacca | |
| | tacgagggcacccagacggcgaaactgaaagtgacgaagggcggcccact | |
| | gcccttcgcctgggacattctgagtccacaatttatgtacggttccaagg | |
| | catacgttaagcaccccgccgacatccctgattacaaaaagctgtcgttc | |
| | ccggaggggtttaaatgggaaagagtgatgaattttgaggacggagggct | |
| | cgtcactgtgactcaagactcctcacttcaagacggcaccctaatttaca | |
| | aggtgaagatgaggggaacaaatttcccccctgatggccctgttatgcag | |
| | aaaaaaacaatgggatgggaggcatccaccgagagactttacccgcgcga | |
| | cggagtgctcaaaggcgaaatccatcaggcactgaaactaaaggacggcg | |
| | gccactacctggttgagttcaaaactatatatatggccaagaaacccgtc | |
| | cagttgccgggatattactatgtggacacaaaactggatataacgtccca | |
| | caacgaagattacaccatagtggaacagtatgagcgcagtgagggtcgtc | |
| | accatctattcctcggacatggcacgggttccacgggctcgggttccagc | |
| | ggcacggcaagctcggaggacaacaacatggcggtgatcaaagagttcat | |
| | gcggtttaaagtccgtatggagggcagcatgaatgggcacgaatttgaaa | |
| | ttgagggagaaggcgagggccgaccatatgaggggacacaaaccgcgaaa | |
| | ctaaaagtcaccaaaggtgggccgcttccttttgcctgggacattctctc | |
| | tcctcagttcatgtatggctccaaggcctacgttaaacatccggctgata | |
| | tccccgattataagaaattgtctttcccggaggggtttaaatgggagcgg | |
| | gttatgaatttcgaggatggtggactggttactgttacacaagattcgag | |
| | tctgcaggatggcacattgatatacaaagtcaaaatgaggggtacgaatt | |
| | ttcccccggacggcccggtgatgcagaaaaaaactatgggctgggaggcc | |
| | agcacggagaggctgtacccgagggacggggttttgaaaggggagataca | |
| | ccaggcgcttaaactgaaggacggggggcactatctggtggaatttaaga | |
| | cgatatatatggcgaaaaagcccgtgcagctacctggctactactacgtc | |
| | gataccaaactcgatattacctctcataacgaggactacactatcgtgga | |
| | gcaatatgaaagatcagaaggacgtcaccacctttttctatacggaatgg | |
| | acgagctttacaaaagttcgccaaagaagaagcggaaggtctaagcttgt | |
| | gtgtcttgtcttatctggttcgtggtggtgagtttgttacaaaaaaatct | |
| | attttccctagttgagatgggaattgaactatctgttgttatgtggattt | |
| | tattttcttttttctctttagaaccttatggttgtgtcaagaagtcttgt | |
| | gtactttagttttatatctctgttttatctcttctattttctttaggatg | |
| | cttgtgatgatgctgtttttttttgtccctaagcaaaaaaatatcatatt | |
| | atatttggtccttggttcatttttttggtttttttttatcttcacatata | |
| | aatattgtttgaatatcttcaatcttttatttgtatgagacaattattta | |
| | agtatcgggtgacaatgcagctattatgtattgtcgattgttcgctggat | |
| | ccgaattcggcactcccagagaggatgcacatgtgaccgagggacacgaa | |
| | gtgatccgtttaaactatcagtgtttgacaggatatattggcgggtaaac | |
| | ctaagagaaaagagcgtttattagaataatcggatatttaaaagggcgtg | |
| | aaaaggtttatccgttcgtccatttgtatgtgccagccgcctttgcgacg | |
| | ctcaccgggctggttgccctcgccgctgggctggcggccgtctatggccc | |
| | tgcaaacgcgccagaaacgccgtcgaagccgtgtgcgagacaccgcggcc | |
| | gccggcgttgtggatacctcgcggaaaacttggccctcactgacagatga | |
| | ggggcggacgttgacacttgaggggccgactcacccggcgcggcgttgac | |
| | agatgaggggcaggctcgatttcggccggcgacgtggagctggccagcct | |
| | cgcaaatcggcgaaaacgcctgattttacgcgagtttcccacagatgatg | |
| | tggacaagcctggggataagtgccctgcggtattgacacttgaggggcgc | |
| | gactactgacagatgaggggcgcgatccttgacacttgaggggcagagtg | |
| | ctgacagatgaggggcgcacctattgacatttgaggggctgtccacaggc | |
| | agaaaatccagcatttgcaagggtttccgcccgtttttcggccaccgcta | |
| | acctgtcttttaacctgcttttaaaccaatatttataaaccttgttttta | |
| | accagggctgcgccctgtgcgcgtgaccgcgcacgccgaaggggggtgcc | |
| | cccccttctcgaaccctcccggcccgctaacgcgggcctcccatcccccc | |
| | aggggctgcgcccctcggccgcgaacggcctcaccccaaaaatggcagcg | |
| | ctggccaattcccgagtgcgcggaacccctatttgtttatttttctaaat | |
| | acattcaaatatgtatccgctcatgagacaataaccctgataaatgcttc | |
| | aataatattgaaaaaggaagagtatggctaaaatgagaatatcaccggaa | |
| | ttgaaaaaactgatcgaaaaataccgctgcgtaaaagatacggaaggaat | |
| | gtctcctgctaaggtatataagctggtgggagaaaatgaaaacctatatt | |
| | taaaaatgacggacagccggtataaagggaccacctatgatgtggaacgg | |
| | gaaaaggacatgatgctatggctggaaggaaagctgcctgttccaaaggt | |
| | cctgcactttgaacggcatgatggctggagcaatctgctcatgagtgagg | |
| | ccgatggcgtcctttgctcggaagagtatgaagatgaacaaagccctgaa | |
| | aagattatcgagctgtatgcggagtgcatcaggctctttcactccatcga | |
| | catatcggattgtccctatacgaatagcttagacagccgcttagccgaat | |
| | tggattacttactgaataacgatctggccgatgtggattgcgaaaactgg | |
| | gaagaggacactccatttaaagatccgcgcgagctgtatgattttttaaa | |
| | gacggaaaagcccgaagaggaacttgtcttttcccacggcgacctgggag | |
| | acagcaacatctttgtgaaagatggcaaagtaagtggctttattgatctt | |
| | gggagaagcggcagggcggacaagtggtatgacattgccttctgcgtccg | |
| | gtcgctcagggaggatatcggggaagaacagtatgtcgagctattttttg | |
| | acttactggggatcaagcctgattgggagaaaataaaatattatatttta | |
| | ctggatgaattgttttagctgtcagaccaagtttactcatatatacttta | |
| | gattgatttaaaacttcatttttaatttaaaaggatctaggtgaagatcc | |
| | tttttgataatctcatgaccaaaatcccttaacgtgagttttcgttccac | |
| | tgagcgtcagaccccgtagaaaagatcaaaggatcttcttgagatccttt | |
| | ttttctgcgcgtaatctgctgcttgcaaacaaaaaaaccaccgctaccag | |
| | cggtggtttgtttgccggatcaagagctaccaactctttttccgaaggta | |
| | actggcttcagcagagcgcagataccaaatactgtccttctagtgtagcc | |
| | gtagttaggccaccacttcaagaactctgtagcaccgcctacatacctcg | |
| | ctctgctaatcctgttaccagtggctgctgccagtggcgataagtcgtgt | |
| | cttaccgggttggactcaagacgatagttaccggataaggcgcagcggtc | |
| | gggctgaacggggggttcgtgcacacagcccagcttggagcgaacgacct | |
| | acaccgaactgagatacctacagcgtgagctatgagaaagcgccacgctt | |
| | cccgaagggagaaaggcggacaggtatccggtaagcggcagggtcggaac | |
| | aggagagcgcacgagggagcttccagggggaaacgcctggtatctttata | |
| | gtcctgtcgggtttcgccacctctgacttgagcgtcgatttttgtgatgc | |
| | tcgtcaggggggcggagcctatggaaaaacgccagcaacgcggccttttt | |
| | acggttcctggcagatcctagatgtggcgcaacgatgccggcgacaagca | |
| | ggagcgcaccgacttcttccgcatcaagtgttttggctctcaggccgagg | |
| | cccacggcaagtatttgggcaaggggtcgctggtattcgtgcagggcaag | |
| | attcggaataccaagtacgagaaggacggccagacggtctacgggaccga | |
| | cttcattgccgataaggtggattatctggacaccaaggcaccaggcgggt | |
| | caaatcaggaataagggcacattgccccggcgtgagtcggggcaatcccg | |
| | caaggagggtgaatgaatcggacgtttgaccggaaggcatacaggcaaga | |
| | actgatcgacgcggggttttccgccgaggatgccgaaaccatcgcaagcc | |
| | gcaccgtcatgcgtgcgccccgcgaaaccttccagtccgtcggctcgatg | |
| | gtccagcaagctacggccaagatcgagcgcgacagcgtgcaactggctcc | |
| | ccctgccctgcccgcgccatcggccgccgtggagcgttcgcgtcgtcttg | |
| | aacaggaggcggcaggtttggcgaagtcgatgaccatcgacacgcgagga | |
| | actatgacgaccaagaagcgaaaaaccgccggcgaggacctggcaaaaca | |
| | ggtcagcgaggccaagcaggccgcgttgctgaaacacacgaagcagcaga | |
| | tcaaggaaatgcagctttccttgttcgatattgcgccgtggccggacacg | |
| | atgcgagcgatgccaaacg | |
| 59 | acacggcccgctctgccctgttcaccacgcgcaacaagaaaatcccgcgc | pTA29b- |
| | gaggcgctgcaaaacaaggtcattttccacgtcaacaaggacgtgaagat | BARNASE |
| | cacctacaccggcgtcgagctgcgggccgacgatgacgaactggtgtggc | CONSTRUCT |
| | agcaggtgttggagtacgcgaagcgcacccctatcggcgagccgatcacc | incorporated |
| | ttcacgttctacgagctttgccaggacctgggctggtcgatcaatggccg | into |
| | gtattacacgaaggccgaggaatgcctgtcgcgcctacaggcgacggcga | plasmid |
| | tgggcttcacgtccgaccgcgttgggcacctggaatcggtgtcgctgctg | vector |
| | caccgcttccgcgtcctggaccgtggcaagaaaacgtcccgttgccaggt | Plasmid |
| | cctgatcgacgaggaaatcgtcgtgctgtttgctggcgaccactacacga | comprising |
| | aattcatatgggagaagtaccgcaagctgtcgccgacggcccgacggatg | construct |
| | ttcgactatttcagctcgcaccgggagccgtacccgctcaagctggaaac | of SEQ ID |
| | cttccgcctcatgtgcggatcggattccacccgcgtgaagaagtggcgcg | NO: 42. |
| | agcaggtcggcgaagcctgcgaagagttgcgaggcagcggcctggtggaa | This one |
| | cacgcctgggtcaatgatgacctggtgcattgcaaacgctagggccttgt | is likely |
| | ggggtcagttccggctgggggttcagcagcccctgctcggatctgttgga | same |
| | ccggacagtagtcatggttgatgggctgcctgtatcgagtggtgattttg | plasmid as |
| | tgccgagctgccggtcggggagctgttggctggctggtggcaggatatat | SEQ ID |
| | tgtggtgtaaacaaattgacgcttagacaacttaataacacattgcggac | NO: 102 |
| | gtttttaatgtactggggttgaacactctgtgccgaattcggatccagcg | |
| | atctggattttagtactggattttggttttaggaattagaaattttattg | |
| | atagaagtattttacaaatacaaatacatactaagggtttcttatatgct | |
| | caacacatgagcgaaaccctataggaaccctaattcccttatctgggaac | |
| | tactcacacattattatggagaaactcgagcttgtcgatcgactctagct | |
| | agagaagctcactttccaacaaccttagtaatttctcccttaacataatg | |
| | aacaaattcttcaagttgatcagctctagaagcaagtctatcttcttctt | |
| | gtccaagataagcttgtctagcttcaagaataactggttgatattgagct | |
| | ggaagtctttccatagcccaatcagcagcaacatcctttggagcaatctt | |
| | tccagtaacagcagaataccaaattctagaaagagtaagaacaacatttc | |
| | tttcatctccagcccaatctggtggagaattccaaagagtaagagtttca | |
| | ttaagagcttcaaaaagatcttgttctggaactggatcaaaaagttcttc | |
| | agcagctggtccaacaagagcaacagaatgttctctagccttagtaagaa | |
| | gaatagcaagatcaatatcaatagtagctggttcaaaaattccagcaaga | |
| | atatcatttctttgccattctccaaattgaagttctctcttagctggata | |
| | tctccatggaataatatcatcatgaacaacaatagtaacttcaacagctc | |
| | taagaatttcagattctcctggagaagcagaagtttcaagaagatcatta | |
| | ataagagctcttctagtagtttcatcaagtctaacagtaacagtaacaag | |
| | aagatcaatatcagaatgtggcttaagtcctccatcaacagcagatccat | |
| | aaagatgaacagcaagaagagtaggttcaagatgtctttcaataactcca | |
| | acaacttcagaaagttgagtagaaacttcagcaataacagcttctctcat | |
| | gcaagcagtagcaacagaagcagaaattctgaaagaacagaacttttgca | |
| | tgaaaatagaatccttcttaagaacaagcatagaattagcagagttctta | |
| | agcttcttagatccgaaaacaagaaaagaagatgacttaggaacttgtgg | |
| | cttatggaaattagagttaggattaagagtttgaattccttgagccatat | |
| | tgttaatttgagccattgtatcgataattgtaaatgtaattgtaatgttg | |
| | tttgttgtttgttgttgttggtaattgttgtaaaaatgagctcttatact | |
| | cgagcgtgtcctctccaaatgaaatgaacttccttatatagaggaagggt | |
| | cttgcgaaggatagtgggattgtgcgtcatcccttacgtcagtggagatg | |
| | tcacatcaatccacttgctttgtagacgtggttggaacctcttctttttc | |
| | cacgatgctcctcgtggggggggtccatctttgggaccactgtcggcaga | |
| | gagatcttgaatgatagcctttcctttatcgcaatgatggcatttgtagg | |
| | agccaccttccttttctactgtcctttcgatgaagtgacagatagctggg | |
| | caatggaatccgaggaggtttcccgaaattatcctttgttgaaaagtctc | |
| | aatagccctttgatcttctgagactgtatctttgacatttttggagtaga | |
| | ccagagtgtcgtgctccaccatgttatcacatcaatccacttgctttgta | |
| | gacgtggttggaacctcttctttttccacgatgctcctcgtggggggggt | |
| | ccatctttgggaccactgtcggcagagagatcttgaatgatagcctttcc | |
| | tttatcgcaatgatggcatttgtaggagccaccttccttttctactgtcc | |
| | tttcgatgaagtgacagatagctgggcaatggaatccgaggaggtttccc | |
| | gaaattatcctttgttgaaaagtctcaatagccctttgatcttctgagac | |
| | tgtatctttgacatttttggagtagaccagagtgtcgtgctccaccatgt | |
| | tgacctccgcaagaattcaagcttagcgttcctaacacctggagaacctt | |
| | ttatgtacttcacaaccctcaatgctgcttccaggtgtgactgtttgggt | |
| | tgctgcatgaattgactcagcacctgcactgcaaagctgatatctggcct | |
| | tgtgattgtcaagtagagaagctttcccaccagtctctgataggatccca | |
| | catcctctaactctgcatcatcagtctttcctaagtgcttgtcatattct | |
| | acagtagtcagcctttggttctgttccattggggttgacactggtctgca | |
| | gcctcccagaccaacacctgatatcagttccagtgcatacttcctctggt | |
| | tcagtaggattcccttttctgatctcagcacctcaatgcctaagaagtat | |
| | tttagttctcccaaatctttcatcttgaaatgctgatgcagggttgcctt | |
| | tgcttctgaaatcaaaacattgctgctgcctgttattaacagatcatcca | |
| | cataaatcaggattatgacaaggtcagtcccctctcttttggtaaacaag | |
| | gagtgatcataagcactttgcataaaaccagcctgcataaggacagtggt | |
| | aagcttgatgttccactgccttgatgcttgctttaaaccatagagggatt | |
| | caacagcctgcacactttggtttccccttggctgtgaaaaccctgaggca | |
| | gagacatataaacttcttccatgaggtcaccttgtagaaaagcattgttg | |
| | acatccatctggaaaaggaaccagcccttggaagcagcaacagatatgac | |
| | agcttttacagtgaccattttggccactggagaaaaagtttcatggtagt | |
| | caaggccttcttgctgagtgtatcccttggccactagccttgccttaaac | |
| | ctgtcaacttcaccattagctttgtatttaattttgtacacccatttgga | |
| | ccctataggctgtttaccagggggtaaagggacaatctcccaggtgttat | |
| | tatcctcaagagcctgtatctcaagggacatggcctccatccatttctca | |
| | tcttgagctgcttctttgaaggatttaggttcagtataagtggagaaagc | |
| | actcaaataagcttgatagtgaacaggcaagtgatcataggaaacatagt | |
| | tggctatagggtatggaacatccctagagcctttgttcagtgtcacaaag | |
| | tccttgagccagatgggaggacctgcattgcgtttaggtctagtgtgcag | |
| | gtttgctggaactagagagggatcagctacagcagtatggtgctcaaatt | |
| | cagcatttgctaggtcaggctcagctgactcaactgactctgcaggagca | |
| | tgcaggtggcctgaaggtgcagcatcagctgaagtgatatgattttgggt | |
| | agaatgagtccccaaagtgatatcctcattggcctctacaatgtcaggaa | |
| | taagaactggagtatcatcatcatcagtatgaaagctggaaggaaaaata | |
| | tcattatacactagctgcaaagctgtatcttcagagctaagtgaacctgc | |
| | tcgagtgaacatatcaggttcatgggagatggactctttgaaagggaact | |
| | gaaactctctgaaaactacatccctgctcactatgatcaccttattatcc | |
| | aagtcatacaacctgtaacccttttgagtctcagaataaccaatgaagat | |
| | ggttcgcttggctctaggtgcaagtttgtcacctttgggcagtgttgctg | |
| | caaaagcaagacacccaaacactctcaagtgatcaagcttggcatgtttc | |
| | tgatagagtagttcatatggacatttgccttgtaagattggagtagggag | |
| | tctattgatcaggtatacaacagtcttgacacagtctccccaaaacctgg | |
| | taggtacaccactctgaaacttaagtgcccttgccatctcaaggatgtgt | |
| | ctgtgctttctctccacaacaccattctgttgtggtgtgtagggacagct | |
| | actttgatgaacaatcccaagagaggccagcaactcattacaacttctcc | |
| | actagaattcgagctcggagtttgtaatattatgatttacgtaaaaatct | |
| | atctaatgattttatttaaactatagttttgaacttttccaactgaaatt | |
| | tcaacacagtaattatggttgcctgtaactcatattttgcaagccccact | |
| | tcaattatgcagcctaacccaaaagtggggatatgtgatttcaaaggata | |
| | tatgcatcaatcatttctccaaattaggaaagtaatttatataaaattaa | |
| | cttgactttaagttagaactgcattttactgaataaaagttataattatt | |
| | aataaataaatggttaagattttaacgttataatatattcatacagtaca | |
| | aattcaagtttttattcttgaaataaaatttttattttaggtatttcatt | |
| | tttatgcagtcatgtgtcttttttttatttttgaaacatattttatggtc | |
| | atttatatcaattcaaattagaattttcttcctctctaactaattattca | |
| | acaacaaaaaaaaactattttttcaagtttttattttactcagtgtttta | |
| | atcaatattaagcataatgagtatttataaatcattctcaaaataatatt | |
| | ttacatgttaaataatttttaagtatttttaagagtgtgtttaattgtgt | |
| | tttaatttcttagtctattttttaaaaactattttatgaaacatttttca | |
| | taaactgatgtttagagagtaaaagtggaaaagtaagatgtgaaaataca | |
| | aggaagaacatgaataatagaaaattaaaagtaaaaaacccaaacatcga | |
| | tcatagagatgttatagttttttggttttgaaaaaactaaaaaaagagat | |
| | agtagttttaaaaacagttttcgaaactatttttatcaaacaacataatt | |
| | tttttagcttttaaataaaaacagctttaaaaaatgtccaggtttttttc | |
| | caaataaattcaattccatattttaataagtaatggttaatccagtggta | |
| | ggtatatattgtttaacacttgaaatttttttccattcaatttttttaat | |
| | atttttcataacacttaatttatttattacatatatcatatgttatatta | |
| | ttatttttctctattctcttcctttctctctagttatttgtaaaaaaaaa | |
| | aacatataatttatttccattgcataaattttatccatgtctatccatgc | |
| | ctatgtttttattctgccacatataccctaagtgttagcaaatatgtgca | |
| | cacattaacctaattaaactatactagaactatatatcggacttgataga | |
| | aagtggagataagccaaggtagacaagaaaaaggattgaacacatgatca | |
| | tcctcatgtcacatgcagttgccatggatccaacaccaaagttatgatca | |
| | gaaaacctctcaaggcaggtgactcacgaaatgaacaagccaatttgctt | |
| | ccaaaattcaccataaatagggacactactcaatgcaaaccatgcataca | |
| | caatcatatacagtgggattagtaaacaaattaattaataaccaaaagtt | |
| | atttaaattgaagtcaatggctcaagttattaatactttcgacggtgttg | |
| | cagactaccttcaaacttatcacaagctgccagataactatatcacaaaa | |
| | tctgaagctcaggccctcggggggtagcttcaaagggtaacctcgccgac | |
| | gttgcacctggaaagagtattgggggcgacatctttagtaaccggtaagt | |
| | ttctgcttctacctttgatatatatataataattatcattaattagtagt | |
| | aatataatatttcaaatatttttttcaaaataaaagaatgtagtatatag | |
| | caattgcttttctgtagtttataagtgtgtatattttaatttataacttt | |
| | tctaatatatgaccaaaatttgttgatgtgcagtgaaggtaaattgcctg | |
| | gaaaatctggacgtacatggcgcgaggccgatataaattacacaagcgga | |
| | ttccgtaactctgacagaatactttattcttcagactggctgatatataa | |
| | aacaacagatcattatcaaacatttactaaaatacgttaggcttgtcaag | |
| | cagatcgttcaaacatttggcaataaagtttcttaagattgaatcctgtt | |
| | gccggtcttgcgatgattatcatataatttctgttgaattacgttaagca | |
| | tgtaataattaacatgtaatgcatgacgttatttatgagatgggttttta | |
| | tgattagagtcccgcaattatacatttaatacgcgatagaaaacaaaata | |
| | tagcgcgcaaactaggataaattatcgcgcgcggtgtcatctatgttact | |
| | agatcgacgctttacgaggatgcacatgtgaccgagggacacgaagtgat | |
| | ccgtttaaactatcagtgtttgacaggatatattggcgggtaaacctaag | |
| | agaaaagagcgtttattagaataatcggatatttaaaagggcgtgaaaag | |
| | gtttatccgttcgtccatttgtatgtgccagccgcctttgcgacgctcac | |
| | cgggctggttgccctcgccgctgggctggcggccgtctatggccctgcaa | |
| | acgcgccagaaacgccgtcgaagccgtgtgcgagacaccgcggccgccgg | |
| | cgttgtggatacctcgcggaaaacttggccctcactgacagatgaggggc | |
| | ggacgttgacacttgaggggccgactcacccggcgcggcgttgacagatg | |
| | aggggcaggctcgatttcggccggcgacgtggagctggccagcctcgcaa | |
| | atcggcgaaaacgcctgattttacgcgagtttcccacagatgatgtggac | |
| | aagcctggggataagtgccctgcggtattgacacttgaggggcgcgacta | |
| | ctgacagatgaggggcgcgatccttgacacttgaggggcagagtgctgac | |
| | agatgaggggcgcacctattgacatttgaggggctgtccacaggcagaaa | |
| | atccagcatttgcaagggtttccgcccgtttttcggccaccgctaacctg | |
| | tcttttaacctgcttttaaaccaatatttataaaccttgtttttaaccag | |
| | ggctgcgccctgtgcgcgtgaccgcgcacgccgaaggggggtgccccccc | |
| | ttctcgaaccctcccggcccgctaacgegggcctcccatccccccagggg | |
| | ctgcgcccctcggccgcgaacggcctcaccccaaaaatggcagcgctggc | |
| | caattcccgagtgcgcggaacccctatttgtttatttttctaaatacatt | |
| | caaatatgtatccgctcatgagacaataaccctgataaatgcttcaataa | |
| | tattgaaaaaggaagagtatggctaaaatgagaatatcaccggaattgaa | |
| | aaaactgatcgaaaaataccgctgcgtaaaagatacggaaggaatgtctc | |
| | ctgctaaggtatataagctggtgggagaaaatgaaaacctatatttaaaa | |
| | atgacggacagccggtataaagggaccacctatgatgtggaacgggaaaa | |
| | ggacatgatgctatggctggaaggaaagctgcctgttccaaaggtcctgc | |
| | actttgaacggcatgatggctggagcaatctgctcatgagtgaggccgat | |
| | ggcgtcctttgctcggaagagtatgaagatgaacaaagccctgaaaagat | |
| | tatcgagctgtatgcggagtgcatcaggctctttcactccatcgacatat | |
| | cggattgtccctatacgaatagcttagacagccgcttagccgaattggat | |
| | tacttactgaataacgatctggccgatgtggattgcgaaaactgggaaga | |
| | ggacactccatttaaagatccgcgcgagctgtatgattttttaaagacgg | |
| | aaaagcccgaagaggaacttgtcttttcccacggcgacctgggagacagc | |
| | aacatctttgtgaaagatggcaaagtaagtggctttattgatcttgggag | |
| | aagcggcagggggacaagtggtatgacattgccttctgcgtccggtcgct | |
| | cagggaggatatcggggaagaacagtatgtcgagctattttttgacttac | |
| | tggggatcaagcctgattgggagaaaataaaatattatattttactggat | |
| | gaattgttttagctgtcagaccaagtttactcatatatactttagattga | |
| | tttaaaacttcatttttaatttaaaaggatctaggtgaagatcctttttg | |
| | ataatctcatgaccaaaatcccttaacgtgagttttcgttccactgagcg | |
| | tcagaccccgtagaaaagatcaaaggatcttcttgagatcctttttttct | |
| | gcgcgtaatctgctgcttgcaaacaaaaaaaccaccgctaccagcggtgg | |
| | tttgtttgccggatcaagagctaccaactctttttccgaaggtaactggc | |
| | ttcagcagagcgcagataccaaatactgtccttctagtgtagccgtagtt | |
| | aggccaccacttcaagaactctgtagcaccgcctacatacctcgctctgc | |
| | taatcctgttaccagtggctgctgccagtggcgataagtcgtgtcttacc | |
| | gggttggactcaagacgatagttaccggataaggcgcagcggtcgggctg | |
| | aacggggggttcgtgcacacagcccagcttggagcgaacgacctacaccg | |
| | aactgagatacctacagcgtgagctatgagaaagcgccacgcttcccgaa | |
| | gggagaaaggcggacaggtatccggtaagcggcagggtcggaacaggaga | |
| | gcgcacgagggagcttccagggggaaacgcctggtatctttatagtcctg | |
| | tcgggtttcgccacctctgacttgagcgtcgatttttgtgatgctcgtca | |
| | ggggggcggagcctatggaaaaacgccagcaacgcggcctttttacggtt | |
| | cctggcagatcctagatgtggcgcaacgatgccggcgacaagcaggagcg | |
| | caccgacttcttccgcatcaagtgttttggctctcaggccgaggcccacg | |
| | gcaagtatttgggcaaggggtcgctggtattcgtgcagggcaagattcgg | |
| | aataccaagtacgagaaggacggccagacggtctacgggaccgacttcat | |
| | tgccgataaggtggattatctggacaccaaggcaccaggcgggtcaaatc | |
| | aggaataagggcacattgccccggcgtgagtcggggcaatcccgcaagga | |
| | gggtgaatgaatcggacgtttgaccggaaggcatacaggcaagaactgat | |
| | cgacgcggggttttccgccgaggatgccgaaaccatcgcaagccgcaccg | |
| | tcatgcgtgcgccccgcgaaaccttccagtccgtcggctcgatggtccag | |
| | caagctacggccaagatcgagcgcgacagcgtgcaactggctccccctgc | |
| | cctgcccgcgccatcggccgccgtggagcgttcgcgtcgtcttgaacagg | |
| | aggggcaggtttggcgaagtcgatgaccatcgacacgcgaggaactatga | |
| | cgaccaagaagcgaaaaaccgccggcgaggacctggcaaaacaggtcagc | |
| | gaggccaagcaggccgcgttgctgaaacacacgaagcagcagatcaagga | |
| | aatgcagctttccttgttcgatattgcgccgtggccggacacgatgcgag | |
| | cgatgccaaacgacacggcccgctctgccctgttcaccacgcgcaacaag | |
| | aaaatcccgcgcgaggcgctgcaaaacaaggtcattttccacgtcaacaa | |
| | ggacgtgaagatcacctacaccggcgtcgagctgcgggccgacgatgacg | |
| | aactggtgtggcagcaggtgttggagtacgcgaagcgcacccctatcggc | |
| | gagccgatcaccttcacgttctacgagctttgccaggacctgggctggtc | |
| | gatcaatggccggtattacacgaaggccgaggaatgcctgtcgcgcctac | |
| | aggcgacggcgatgggcttcacgtccgaccgcgttgggcacctggaatcg | |
| | gtgtcgctgctgcaccgcttccgcgtcctggaccgtggcaagaaaacgtc | |
| | ccgttgccaggtcctgatcgacgaggaaatcgtcgtgctgtttgctggcg | |
| | accactacacgaaattcatatgggagaagtaccgcaagctgtcgccgacg | |
| | gcccgacggatgttcgactatttcagctcgcaccgggagccgtacccgct | |
| | caagctggaaaccttccgcctcatgtgcggatcggattccacccgcgtga | |
| | agaagtggcgcgagcaggtcggcgaagcctgcgaagagttgcgaggcagc | |
| | ggcctggtggaacacgcctgggtcaatgatgacctggtgcattgcaaacg | |
| | ctagggccttgtggggtcagttccggctgggggttcagcagcccctgctc | |
| | ggatctgttggaccggacagtagtcatggttgatgggctgcctgtatcga | |
| | gtggtgattttgtgccgagctgccggtcggggagctgttggctggctggt | |
| | ggcaggatatattgtggtgtaaacaaattgacgcttagacaacttaataa | |
| | cacattgcggacgtttttaatgtactggggttgaacactct | |
| 60 | gtgccgaattcggatccagcgatctggattttagtactggattttggttt | pTA29a:: |
| | taggaattagaaattttattgatagaagtattttacaaatacaaatacat | BARSTAR |
| | actaagggtttcttatatgctcaacacatgagcgaaaccctataggaacc | Plasmid |
| | ctaattcccttatctgggaactactcacacattattatggagaaactcga | comprising |
| | gcttgtcgatcgactctagctagagaagctcactttccaacaaccttagt | construct |
| | aatttctcccttaacataatgaacaaattcttcaagttgatcagctctag | of SEQ ID |
| | aagcaagtctatcttcttcttgtccaagataagcttgtctagcttcaaga | NO: 43. |
| | ataactggttgatattgagctggaagtctttccatagcccaatcagcagc | This one |
| | aacatcctttggagcaatctttccagtaacagcagaataccaaattctag | is likely |
| | aaagagtaagaacaacatttctttcatctccagcccaatctggtggagaa | same |
| | ttccaaagagtaagagtttcattaagagcttcaaaaagatcttgttctgg | plasmid as |
| | aactggatcaaaaagttcttcagcagctggtccaacaagagcaacagaat | SEQ ID |
| | gttctctagccttagtaagaagaatagcaagatcaatatcaatagtagct | NO: 101 |
| | ggttcaaaaattccagcaagaatatcatttctttgccattctccaaattg | |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggaggtttccata | |
| | gcctaaggccacgagtgcacgtgttcataggctagttgtgttaaccttgg | |
| | ggagcagtcgacaatttggattgcatcgaggtcttgccaaattttacttt | |
| | caagacagtgatttcgacggcacaataaacttccaattagttatcttctc | |
| | ttttgttctgtattgtgtttttttctatttttctaacaatggaaatagta | |
| | tataccggccaacagtgttgcactgttgtctggaccttgtcaaaggttcg | |
| | acagtgcatagtttggactcgatgtggcatatgggggttctgataagtga | |
| | tgagattatgaaaataatttaattttagctattaaaaatacaatacaact | |
| | ttcaaaagcgggaaacttgtttgattatgatctaggattctaggtctttt | |
| | tttttaagttcgagacagaaaaccaaaaacaaaacagctaataagaagaa | |
| | gaaaaacaatacagcaatattcccacaaaaataaatttgttcttttgcgt | |
| | actagccataggatttaattatatatatgggccagtccagaagcattgtc | |
| | actgatagatcatagactagtaaaaaagaatgaaccacttcagaacacta | |
| | actatatatataaagatagattcatatctatttagatcgtgattattaat | |
| | acaagaaaatatttgtatttcttaattttttgtttttaaatttatgtaaa | |
| | tatattgacatcttttttaacgaaaattcaaattttaattcattatatta | |
| | caagaaattaacacattttactagaccaactctcatgttacaacactata | |
| | tatgttgaacattacaagaatgccactttatctcacccctttaattagga | |
| | aaagcaaaaatctatttgcatataattttgtaccaaaaaattatacaata | |
| | aaaaagaaaaagagaaaagtaaaagataagacgactgatgtaataaaaag | |
| | agagaaaaaataatactaaaaaataatataaataacataactcttaaaaa | |
| | aaaaaggtaacactgttgagtattttttttattacttttctctaaaaagg | |
| | tatcatggtttcactaatgtccaggttttttttttccaaataaactcctt | |
| | tccggtggtaggtatcgtttcacacttgaatacttttttgcaatattttt | |
| | cgtaacactttatttctctccattacataattttacccttgcctatgtct | |
| | ttattctgccacatataccctaagagtgagcaaatgtgtgcacgcatata | |
| | acctgattggactatatatcggacttgatacaaatgtgtgctcacatgaa | |
| | gagaaaaaggattgaacccatgatcatcatcatgtcacatgcagatgcca | |
| | tgcattcaacaccaaagttatgatcagaaaacctctcaaggtaggtgact | |
| | caccaaatgaacaagccaattttttgcttcccaaatttcaccataaatag | |
| | ggacactactccatccaaaccatacacgaccacatacagtgggattagta | |
| | agtaaacaaattaattaataaccaaaagttataatttcaattgaatttag | |
| | tcaatgaagaaggccgtcataaatggagaacagatcaggagtatcagcga | |
| | tctccatcaaactctgaagaaagaactcgctctgccagagtattacggtg | |
| | aaaaccttgatgctctttgggactgcctcactggttgggtcgagtatcca | |
| | ctcgtcctggaatggagacagttcgaacaatccaaacagctcaccgagaa | |
| | tggagctgaaagtgttttgcaagtctttagggaagcaaaagccgaaggct | |
| | gtgacatcaccattatactctcttaggcttgtcaagcagatcgttcaaac | |
| | atttggcaataaagtttcttaagattgaatcctgttgccggtcttgcgat | |
| | gattatcatataatttctgttgaattacgttaagcatgtaataattaaca | |
| | tgtaatgcatgacgttatttatgagatgggtttttatgattagagtcccg | |
| | caattatacatttaatacgcgatagaaaacaaaatatagcgcgcaaacta | |
| | ggataaattatcgcgcgcggtgtcatctatgttactagatcgacgcttta | |
| | cgaattcccatggagcgtccctcggtcacatgtgcatcctctagtggaga | |
| | agttgtaatgagttgctggcctctcttgggattgttcatcaaagtagctg | |
| | tccctacacaccacaacagaatggtgttgtggagagaaagcacagacaca | |
| | tccttgagatggcaagggcacttaagtttcagagtggtgtacctaccagg | |
| | ttttggggagactgtgtcaagactgttgtatacctgatcaatagactccc | |
| | tactccaatcttacaaggcaaatgtccatatgaactactctatcagaaac | |
| | atgccaagcttgatcacttgagagtgtttgggtgtcttgcttttgcagca | |
| | acactgcccaaaggtgacaaacttgcacctagagccaagcgaaccatctt | |
| | cattggttattctgagactcaaaagggttacaggttgtatgacttggata | |
| | ataaggtgatcatagtgagcagggatgtagttttcagagagtttcagttc | |
| | cctttcaaagagtccatctcccatgaacctgatatgttcactcgagcagg | |
| | ttcacttagctctgaagatacagctttgcagctagtgtataatgatattt | |
| | ttccttccagctttcatactgatgatgatgatactccagttcttattcct | |
| | gacattgtagaggccaatgaggatatcactttggggactcattctaccca | |
| | aaatcatatcacttcagctgatgctgcaccttcaggccacctgcatgctc | |
| | ctgcagagtcagttgagtcagctgagcctgacctagcaaatgctgaattt | |
| | gagcaccatactgctgtagctgatccctctctagttccagcaaacctgca | |
| | cactagacctaaacgcaatgcaggtcctcccatctggctcaaggactttg | |
| | tgacactgaacaaaggctctagggatgttccataccctatagccaactat | |
| | gtttcctatgatcacttgcctgttcactatcaagcttatttgagtgcttt | |
| | ctccacttatactgaacctaaatccttcaaagaagcagctcaagatgaga | |
| | aatggatggaggccatgtcccttgagatacaggctcttgaggataataac | |
| | acctgggagattgtccctttaccccctggtaaacagcctatagggtccaa | |
| | atgggtgtacaaaattaaatacaaagctaatggtgaagttgacaggttta | |
| | aggcaaggctagtggccaagggatacactcagcaagaaggccttgactac | |
| | catgaaactttttctccagtggccaaaatggtcactgtaaaagctgtcat | |
| | atctgttgctgcttccaagggctggttccttttccagatggatgtcaaca | |
| | atgcttttctacaaggtgacctcatggaagaagtttatatgtctctgcct | |
| | cagggttttcacagccaaggggaaaccaaagtgtgcaggctgttgaatcc | |
| | ctctatggtttaaagcaagcatcaaggcagtggaacatcaagcttaccac | |
| | tgtccttatgcaggctggttttatgcaaagtgcttatgatcactccttgt | |
| | ttaccaaaagagaggggactgaccttgtcataatcctgatttatgtggat | |
| | gatctgttaataacaggcagcagcaatgttttgatttcagaagcaaaggc | |
| | aaccctgcatcagcatttcaagatgaaagatttgggagaactaaaatact | |
| | tcttaggcattgaggtgctgagatcagaaaagggaatcctactgaaccag | |
| | aggaagtatgcactggaactgatatcaggtgttggtctgggaggctgcag | |
| | accagtgtcaaccccaatggaacagaaccaaaggctgactactgtagaat | |
| | atgacaagcacttaggaaagactgatgatgcagagttagaggatgtggga | |
| | tcctatcagagactggtgggaaagcttctctacttgacaatcacaaggcc | |
| | agatatcagctttgcagtgcaggtgctgagtcaattcatgcagcaaccca | |
| | aacagtcacacctggaagcagcattgagggttgtgaagtacataaaaggt | |
| | tctccaggtgttaggaacgctaagcttgaattcttgcggaggacgtcgtt | |
| | gtggttggtgctttccttacattctgagcctctttccttctaatccactc | |
| | atctgcatcttcttgtgtccttactaatacctcattggttccaaattccc | |
| | tccctttaagcaccagctcgtttctgttcttccacagcctcccaagtatc | |
| | caagggactaaagcctccacattcttcagatcaggatattcttgtttaag | |
| | atgttgaactctatggaggtttgtatgaactgatgatctaggaccggata | |
| | agttcccttcttcatagcgaacttattcaaagaatgttttgtgtatcatt | |
| | cttgttacattgttattaatgaaaaaatattattggtcattggactgaac | |
| | acgagtgttaaatatggaccaggccccaaataagatccattgatatatga | |
| | attaaataacaagaataaatcgagtcaccaaaccacttgccttttttaac | |
| | gagacttgttcaccaacttgatacaaaagtcattatcctatgcaaatcaa | |
| | taatcatacaaaaatatccaataacactaaaaaaattaaaagaaatggat | |
| | aatttcacaatatgttatacgataaagaagttacttttccaagaaattca | |
| | ctgattttataagcccacttgcattagataaatggcaaaaaaaaacaaaa | |
| | aggaaaagaaataaagcacgaagaattctagaaaatacgaaatacgcttc | |
| | aatgcagtgggacccacggttcaattattgccaattttcagctccaccgt | |
| | atatttaaaaaataaaacgataatgctaaaaaaatataaatcgtaacgat | |
| | cgttaaatctcaacggctggatcttatgacgaccgttaaggaaattgtgg | |
| | ttgtcggacgaagtccagtaataaacggcgtcaaagtggttgcagccggc | |
| | acacacgagtcgtgtttatcaactcaaagcacaaatacttttcctcaacc | |
| | taaaaataaggcaattagccaaaaacaactttgcgtgtaaacaacgctca | |
| | atacacgtgtcattttattattagctattgcttcaccgccttagctttct | |
| | cgtgacctagtcgtcctcgtcttttcttcttcttcttctataaaacaata | |
| | cccaaagagctcttcttcttcacaattcagatttcaatttctcaaaatct | |
| | taaaaactttctctcaattctctctaccgtgatcaaggtaaatttctgtg | |
| | ttccttattctctcaaaatcttcgattttgttttcgttcgatcccaattt | |
| | cgtatatgttctttggtttagattctgttaatcttagatcgtagacgatt | |
| | ttctgggtttgatcgttagatatcatcttaattctcgattagggtttcat | |
| | agatatcatccgatttgttcaaataatttgagttttgtcgaataattact | |
| | cttcgatttgtgatttctatctagatctggtgttagtttctagtttgtgc | |
| | gatcgaatttgtcgattaatctgagtttttctgattaacagatgcagatc | |
| | ttaatggtcaagactaatcttttccttttcttcatcttctcgcttctcct | |
| | ctcactttccagcgcggacatggtgtccaagggcgaagaggtgattaaag | |
| | agttcatgaggttcaaagtgagaatggagggcagtatgaacggccatgag | |
| | ttcgagatcgaaggtgaaggcgaaggtagaccatacgagggcacccagac | |
| | ggcgaaactgaaagtgacgaagggcggcccactgcccttcgcctgggaca | |
| | ttctgagtccacaatttatgtacggttccaaggcatacgttaagcacccc | |
| | gccgacatccctgattacaaaaagctgtcgttcccggaggggtttaaatg | |
| | ggaaagagtgatgaattttgaggacggagggctcgtcactgtgactcaag | |
| | actcctcacttcaagacggcaccctaatttacaaggtgaagatgagggga | |
| | acaaatttcccccctgatggccctgttatgcagaaaaaaacaatgggatg | |
| | ggaggcatccaccgagagactttacccgcgcgacggagtgctcaaaggcg | |
| | aaatccatcaggcactgaaactaaaggacggcggccactacctggttgag | |
| | ttcaaaactatatatatggccaagaaacccgtccagttgccgggatatta | |
| | ctatgtggacacaaaactggatataacgtcccacaacgaagattacacca | |
| | tagtggaacagtatgagcgcagtgagggtcgtcaccatctattcctcgga | |
| | catggcacgggttccacgggctcgggttccageggcacggcaagctcgga | |
| | ggacaacaacatggcggtgatcaaagagttcatgcggtttaaagtccgta | |
| | tggagggcagcatgaatgggcacgaatttgaaattgagggagaaggcgag | |
| | ggccgaccatatgaggggacacaaaccgcgaaactaaaagtcaccaaagg | |
| | tgggccgcttccttttgcctgggacattctctctcctcagttcatgtatg | |
| | gctccaaggcctacgttaaacatccggctgatatccccgattataagaaa | |
| | ttgtctttcccggaggggtttaaatgggagcgggttatgaatttcgagga | |
| | tggtggactggttactgttacacaagattcgagtctgcaggatggcacat | |
| | tgatatacaaagtcaaaatgaggggtacgaattttcccccggacggcccg | |
| | gtgatgcagaaaaaaactatgggctgggaggccagcacggagaggctgta | |
| | cccgagggacggggttttgaaaggggagatacaccaggcgcttaaactga | |
| | aggacggggggcactatctggtggaatttaagacgatatatatggcgaaa | |
| | aagcccgtgcagctacctggctactactacgtcgataccaaactcgatat | |
| | tacctctcataacgaggactacactatcgtggagcaatatgaaagatcag | |
| | aaggacgtcaccacctttttctatacggaatggacgagctttacaaaaag | |
| | gacgagttgtaagcttgtgtgtcttgtcttatctggttcgtggtggtgag | |
| | tttgttacaaaaaaatctattttccctagttgagatgggaattgaactat | |
| | ctgttgttatgtggattttattttcttttttctctttagaaccttatggt | |
| | tgtgtcaagaagtcttgtgtactttagttttatatctctgttttatctct | |
| | tctattttctttaggatgcttgtgatgatgctgtttttttttgtccctaa | |
| | gcaaaaaaatatcatattatatttggtccttggttcatttttttggtttt | |
| | tttttatcttcacatataaatattgtttgaatatcttcaatcttttattt | |
| | gtatgagacaattatttaagtatcgggtgacaatgcagctattatgtatt | |
| | gtcgattgttcgctggatccgaattcggcactcccagagaggatgcacat | |
| | gtgaccgagggacacgaagtgatccgtttaaactatcagtgtttgacagg | |
| | atatattggcgggtaaacctaagagaaaagagcgtttattagaataatcg | |
| | gatatttaaaagggcgtgaaaaggtttatccgttcgtccatttgtatgtg | |
| | ccagccgcctttgcgacgctcaccgggctggttgccctcgccgctgggct | |
| | ggeggccgtctatggccctgcaaacgcgccagaaacgccgtcgaagccgt | |
| | gtgcgagacaccgcggccgccggcgttgtggatacctcgcggaaaacttg | |
| | gccctcactgacagatgaggggcggacgttgacacttgaggggccgactc | |
| | acccggcgcggcgttgacagatgaggggcaggctcgatttcggccggcga | |
| | cgtggagctggccagcctcgcaaatcggcgaaaacgcctgattttacgcg | |
| | agtttcccacagatgatgtggacaagcctggggataagtgccctgcggta | |
| | ttgacacttgaggggcgcgactactgacagatgaggggcgcgatccttga | |
| | cacttgaggggcagagtgctgacagatgaggggcgcacctattgacattt | |
| | gaggggctgtccacaggcagaaaatccagcatttgcaagggtttccgccc | |
| | gttttteggccaccgctaacctgtcttttaacctgcttttaaaccaatat | |
| | ttataaaccttgtttttaaccagggctgcgccctgtgcgcgtgaccgcgc | |
| | acgccgaaggggggtgcccccccttctcgaaccctcccggcccgctaacg | |
| | cgggcctcccatccccccaggggctgcgcccctcggccgcgaacggcctc | |
| | accccaaaaatggcagcgctggccaattcccgagtgcgcggaacccctat | |
| | ttgtttatttttctaaatacattcaaatatgtatccgctcatgagacaat | |
| | aaccctgataaatgcttcaataatattgaaaaaggaagagtatggctaaa | |
| | atgagaatatcaccggaattgaaaaaactgatcgaaaaataccgctgcgt | |
| | aaaagatacggaaggaatgtctcctgctaaggtatataagctggtgggag | |
| | aaaatgaaaacctatatttaaaaatgacggacagccggtataaagggacc | |
| | acctatgatgtggaacgggaaaaggacatgatgctatggctggaaggaaa | |
| | gctgcctgttccaaaggtcctgcactttgaacggcatgatggctggagca | |
| | atctgctcatgagtgaggccgatggcgtcctttgctcggaagagtatgaa | |
| | gatgaacaaagccctgaaaagattatcgagctgtatgcggagtgcatcag | |
| | gctctttcactccatcgacatatcggattgtccctatacgaatagcttag | |
| | acagccgcttagccgaattggattacttactgaataacgatctggccgat | |
| | gtggattgcgaaaactgggaagaggacactccatttaaagatccgcgcga | |
| | gctgtatgattttttaaagacggaaaagcccgaagaggaacttgtctttt | |
| | cccacggcgacctgggagacagcaacatctttgtgaaagatggcaaagta | |
| | agtggctttattgatcttgggagaagcggcagggcggacaagtggtatga | |
| | cattgccttctgcgtccggtcgctcagggaggatatcggggaagaacagt | |
| | atgtcgagctattttttgacttactggggatcaagcctgattgggagaaa | |
| | ataaaatattatattttactggatgaattgttttagctgtcagaccaagt | |
| | ttactcatatatactttagattgatttaaaacttcatttttaatttaaaa | |
| | ggatctaggtgaagatcctttttgataatctcatgaccaaaatcccttaa | |
| | cgtgagttttcgttccactgagcgtcagaccccgtagaaaagatcaaagg | |
| | atcttcttgagatcctttttttctgcgcgtaatctgctgcttgcaaacaa | |
| | aaaaaccaccgctaccagcggtggtttgtttgccggatcaagagctacca | |
| | actctttttccgaaggtaactggcttcagcagagcgcagataccaaatac | |
| | tgtccttctagtgtagccgtagttaggccaccacttcaagaactctgtag | |
| | caccgcctacatacctcgctctgctaatcctgttaccagtggctgctgcc | |
| | agtggcgataagtcgtgtcttaccgggttggactcaagacgatagttacc | |
| | ggataaggcgcagcggtcgggctgaacggggggttcgtgcacacagccca | |
| | gcttggagcgaacgacctacaccgaactgagatacctacagcgtgagcta | |
| | tgagaaagcgccacgcttcccgaagggagaaaggcggacaggtatccggt | |
| | aagcggcagggtcggaacaggagagcgcacgagggagcttccagggggaa | |
| | acgcctggtatctttatagtcctgtcgggtttcgccacctctgacttgag | |
| | cgtcgatttttgtgatgctcgtcaggggggcggagcctatggaaaaacgc | |
| | cagcaacgcggcctttttacggttcctggcagatcctagatgtggcgcaa | |
| | cgatgccggcgacaagcaggagcgcaccgacttcttccgcatcaagtgtt | |
| | ttggctctcaggccgaggcccacggcaagtatttgggcaaggggtcgctg | |
| | gtattcgtgcagggcaagattcggaataccaagtacgagaaggacggcca | |
| | gacggtctacgggaccgacttcattgccgataaggtggattatctggaca | |
| | ccaaggcaccaggcgggtcaaatcaggaataagggcacattgccccggcg | |
| | tgagtcggggcaatcccgcaaggagggtgaatgaatcggacgtttgaccg | |
| | gaaggcatacaggcaagaactgatcgacgcggggttttccgccgaggatg | |
| | ccgaaaccatcgcaagccgcaccgtcatgegtgcgccccgcgaaaccttc | |
| | cagtccgtcggctcgatggtccagcaagctacggccaagatcgagcgcga | |
| | cagcgtgcaactggetccccctgccctgcccgcgccatcggccgccgtgg | |
| | agcgttcgcgtcgtcttgaacaggaggcggcaggtttggcgaagtcgatg | |
| | accatcgacacgcgaggaactatgacgaccaagaagcgaaaaaccgccgg | |
| | cgaggacctggcaaaacaggtcagcgaggccaagcaggccgcgttgctga | |
| | aacacacgaagcagcagatcaaggaaatgcagctttccttgttcgatatt | |
| | gcgccgtggccggacacgatgcgagcgatgccaaacgacacggcccgctc | |
| | tgccctgttcaccacgcgcaacaagaaaatcccgcgcgaggcgctgcaaa | |
| | acaaggtcattttccacgtcaacaaggacgtgaagatcacctacaccggc | |
| | gtcgagctgcgggccgacgatgacgaactggtgtggcagcaggtgttgga | |
| | gtacgcgaagcgcacccctatcggcgagccgatcaccttcacgttctacg | |
| | agctttgccaggacctgggctggtcgatcaatggccggtattacacgaag | |
| | gccgaggaatgcctgtcgcgcctacaggcgacggcgatgggcttcacgtc | |
| | cgaccgcgttgggcacctggaatcggtgtcgctgctgcaccgcttccgcg | |
| | tcctggaccgtggcaagaaaacgtcccgttgccaggtcctgatcgacgag | |
| | gaaatcgtcgtgctgtttgctggcgaccactacacgaaattcatatggga | |
| | gaagtaccgcaagctgtcgccgacggcccgacggatgttcgactatttca | |
| | gctcgcaccgggagccgtacccgctcaagctggaaaccttccgcctcatg | |
| | tgcggatcggattccacccgcgtgaagaagtggcgcgagcaggtcggcga | |
| | agcctgcgaagagttgcgaggcagcggcctggtggaacacgcctgggtca | |
| | atgatgacctggtgcattgcaaacgctagggccttgtggggtcagttccg | |
| | gctgggggttcagcagcccctgctcggatctgttggaccggacagtagtc | |
| | atggttgatgggctgcctgtatcgagtggtgattttgtgccgagctgccg | |
| | gtcggggagctgttggctggctggtggcaggatatattgtggtgtaaaca | |
| | aattgacgcttagacaacttaataacacattgcggacgtttttaatgtac | |
| | tggggttgaacactct | |
| 61 | gtgccgaattcggatccagcgatctggattttagtactggattttggttt | pTA29b:: |
| | taggaattagaaattttattgatagaagtattttacaaatacaaatacat | BARSTAR |
| | actaagggtttcttatatgctcaacacatgagcgaaaccctataggaacc | Plasmid |
| | ctaattcccttatctgggaactactcacacattattatggagaaactcga | comprising |
| | gcttgtcgatcgactctagctagagaagctcactttccaacaaccttagt | construct |
| | aatttctcccttaacataatgaacaaattcttcaagttgatcagctctag | of SEQ ID |
| | aagcaagtctatcttcttcttgtccaagataagcttgtctagcttcaaga | NO: 44. |
| | ataactggttgatattgagctggaagtctttccatagcccaatcagcagc | This one |
| | aacatcctttggagcaatctttccagtaacagcagaataccaaattctag | is likely |
| | aaagagtaagaacaacatttctttcatctccagcccaatctggtggagaa | same |
| | ttccaaagagtaagagtttcattaagagcttcaaaaagatcttgttctgg | plasmid as |
| | aactggatcaaaaagttcttcagcagctggtccaacaagagcaacagaat | SEQ ID |
| | gttctctagccttagtaagaagaatagcaagatcaatatcaatagtagct | NO: 100 |
| | ggttcaaaaattccagcaagaatatcatttctttgccattctccaaattg | |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtectttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagtttgtaata | |
| | ttatgatttacgtaaaaatctatctaatgattttatttaaactatagttt | |
| | tgaacttttccaactgaaatttcaacacagtaattatggttgcctgtaac | |
| | tcatattttgcaagccccacttcaattatgcagcctaacccaaaagtggg | |
| | gatatgtgatttcaaaggatatatgcatcaatcatttctccaaattagga | |
| | aagtaatttatataaaattaacttgactttaagttagaactgcattttac | |
| | tgaataaaagttataattattaataaataaatggttaagattttaacgtt | |
| | ataatatattcatacagtacaaattcaagtttttattcttgaaataaaat | |
| | ttttattttaggtatttcatttttatgcagtcatgtgtcttttttttatt | |
| | tttgaaacatattttatggtcatttatatcaattcaaattagaattttct | |
| | tcctctctaactaattattcaacaacaaaaaaaaactattttttcaagtt | |
| | tttattttactcagtgttttaatcaatattaagcataatgagtatttata | |
| | aatcattctcaaaataatattttacatgttaaataatttttaagtatttt | |
| | taagagtgtgtttaattgtgttttaatttcttagtctattttttaaaaac | |
| | tattttatgaaacatttttcataaactgatgtttagagagtaaaagtgga | |
| | aaagtaagatgtgaaaatacaaggaagaacatgaataatagaaaattaaa | |
| | agtaaaaaacccaaacatcgatcatagagatgttatagttttttggtttt | |
| | gaaaaaactaaaaaaagagatagtagttttaaaaacagttttcgaaacta | |
| | tttttatcaaacaacataatttttttagcttttaaataaaaacagcttta | |
| | aaaaatgtccaggtttttttccaaataaattcaattccatattttaataa | |
| | gtaatggttaatccagtggtaggtatatattgtttaacacttgaaatttt | |
| | tttccattcaatttttttaatatttttcataacacttaatttatttatta | |
| | catatatcatatgttatattattatttttctctattctcttcctttctct | |
| | ctagttatttgtaaaaaaaaaaacatataatttatttccattgcataaat | |
| | tttatccatgtctatccatgcctatgtttttattctgccacatataccct | |
| | aagtgttagcaaatatgtgcacacattaacctaattaaactatactagaa | |
| | ctatatatcggacttgatagaaagtggagataagccaaggtagacaagaa | |
| | aaaggattgaacacatgatcatcctcatgtcacatgcagttgccatggat | |
| | ccaacaccaaagttatgatcagaaaacctctcaaggcaggtgactcacga | |
| | aatgaacaagccaatttgcttccaaaattcaccataaatagggacactac | |
| | tcaatgcaaaccatgcatacacaatcatatacagtgggattagtaaacaa | |
| | attaattaataaccaaaagttatttaaattgaagtcaatgaagaaggccg | |
| | tcataaatggagaacagatcaggagtatcagcgatctccatcaaactctg | |
| | aagaaagaactcgctctgccagagtattacggtgaaaaccttgatgctct | |
| | ttgggactgcctcactggttgggtcgagtatccactcgtcctggaatgga | |
| | gacagttcgaacaatccaaacagctcaccgagaatggagctgaaagtgtt | |
| | ttgcaagtctttagggaagcaaaagccgaaggctgtgacatcaccattat | |
| | actctcttaggcttgtcaagcagatcgttcaaacatttggcaataaagtt | |
| | tcttaagattgaatcctgttgccggtcttgcgatgattatcatataattt | |
| | ctgttgaattacgttaagcatgtaataattaacatgtaatgcatgacgtt | |
| | atttatgagatgggtttttatgattagagtcccgcaattatacatttaat | |
| | acgcgatagaaaacaaaatatagcgcgcaaactaggataaattatcgcgc | |
| | gcggtgtcatctatgttactagatcgacgctttacgaggatgcacatgtg | |
| | accgagggacacgaagtgatccgtttaaactatcagtgtttgacaggata | |
| | tattggcgggtaaacctaagagaaaagagcgtttattagaataatcggat | |
| | atttaaaagggcgtgaaaaggtttatccgttcgtccatttgtatgtgcca | |
| | gccgcctttgcgacgctcaccgggctggttgccctcgccgctgggctggc | |
| | ggccgtctatggccctgcaaacgcgccagaaacgccgtcgaagccgtgtg | |
| | cgagacaccgcggccgccggcgttgtggatacctcgcggaaaacttggcc | |
| | ctcactgacagatgaggggcggacgttgacacttgaggggccgactcacc | |
| | cggcgcggcgttgacagatgaggggcaggctcgatttcggccggcgacgt | |
| | ggagctggccagcctcgcaaatcggcgaaaacgcctgattttacgcgagt | |
| | ttcccacagatgatgtggacaagcctggggataagtgccctgcggtattg | |
| | acacttgaggggcgcgactactgacagatgaggggcgcgatccttgacac | |
| | ttgaggggcagagtgctgacagatgaggggcgcacctattgacatttgag | |
| | gggctgtccacaggcagaaaatccagcatttgcaagggtttccgcccgtt | |
| | tttcggccaccgctaacctgtcttttaacctgcttttaaaccaatattta | |
| | taaaccttgtttttaaccagggctgcgccctgtgcgcgtgaccgcgcacg | |
| | ccgaaggggggtgcccccccttctegaaccctcccggcccgctaacgcgg | |
| | gcctcccatccccccaggggctgcgcccctcggccgcgaacggcctcacc | |
| | ccaaaaatggcagcgctggccaattcccgagtgcgcggaacccctatttg | |
| | tttatttttctaaatacattcaaatatgtatccgctcatgagacaataac | |
| | cctgataaatgcttcaataatattgaaaaaggaagagtatggctaaaatg | |
| | agaatatcaccggaattgaaaaaactgatcgaaaaataccgctgcgtaaa | |
| | agatacggaaggaatgtctcctgctaaggtatataagctggtgggagaaa | |
| | atgaaaacctatatttaaaaatgacggacagccggtataaagggaccacc | |
| | tatgatgtggaacgggaaaaggacatgatgctatggctggaaggaaagct | |
| | gcctgttccaaaggtcctgcactttgaacggcatgatggctggagcaatc | |
| | tgctcatgagtgaggccgatggcgtcctttgctcggaagagtatgaagat | |
| | gaacaaagccctgaaaagattatcgagctgtatgcggagtgcatcaggct | |
| | ctttcactccatcgacatatcggattgtccctatacgaatagcttagaca | |
| | gccgcttagccgaattggattacttactgaataacgatctggccgatgtg | |
| | gattgcgaaaactgggaagaggacactccatttaaagatccgcgcgagct | |
| | gtatgattttttaaagacggaaaagcccgaagaggaacttgtcttttccc | |
| | acggcgacctgggagacagcaacatctttgtgaaagatggcaaagtaagt | |
| | ggctttattgatcttgggagaagcggcagggcggacaagtggtatgacat | |
| | tgccttctgcgtccggtcgctcagggaggatatcggggaagaacagtatg | |
| | tcgagctattttttgacttactggggatcaagcctgattgggagaaaata | |
| | aaatattatattttactggatgaattgttttagctgtcagaccaagttta | |
| | ctcatatatactttagattgatttaaaacttcatttttaatttaaaagga | |
| | tctaggtgaagatcctttttgataatctcatgaccaaaatcccttaacgt | |
| | gagttttcgttccactgagcgtcagaccccgtagaaaagatcaaaggatc | |
| | ttcttgagatcctttttttctgcgcgtaatctgctgcttgcaaacaaaaa | |
| | aaccaccgctaccagcggtggtttgtttgccggatcaagagctaccaact | |
| | ctttttccgaaggtaactggcttcagcagagcgcagataccaaatactgt | |
| | ccttctagtgtagccgtagttaggccaccacttcaagaactctgtagcac | |
| | cgcctacatacctcgctctgctaatcctgttaccagtggctgctgccagt | |
| | ggcgataagtcgtgtcttaccgggttggactcaagacgatagttaccgga | |
| | taaggcgcagcggtcgggctgaacggggggttcgtgcacacagcccagct | |
| | tggagcgaacgacctacaccgaactgagatacctacagcgtgagctatga | |
| | gaaagcgccacgcttcccgaagggagaaaggcggacaggtatccggtaag | |
| | cggcagggtcggaacaggagagcgcacgagggagcttccagggggaaacg | |
| | cctggtatctttatagtcctgtcgggtttcgccacctctgacttgagcgt | |
| | cgatttttgtgatgctcgtcaggggggcggagcctatggaaaaacgccag | |
| | caacgcggcctttttacggttcctggcagatcctagatgtggcgcaacga | |
| | tgccggcgacaagcaggagcgcaccgacttcttccgcatcaagtgttttg | |
| | gctctcaggccgaggcccacggcaagtatttgggcaaggggtcgctggta | |
| | ttcgtgcagggcaagattcggaataccaagtacgagaaggacggccagac | |
| | ggtctacgggaccgacttcattgccgataaggtggattatctggacacca | |
| | aggcaccagggggtcaaatcaggaataagggcacattgccccggcgtgag | |
| | tcggggcaatcccgcaaggagggtgaatgaatcggacgtttgaccggaag | |
| | gcatacaggcaagaactgatcgacgcggggttttccgccgaggatgccga | |
| | aaccatcgcaagccgcaccgtcatgcgtgcgccccgcgaaaccttccagt | |
| | ccgtcggctcgatggtccagcaagctacggccaagatcgagcgcgacagc | |
| | gtgcaactggctccccctgccctgcccgcgccateggccgccgtggagcg | |
| | ttcgcgtcgtcttgaacaggaggcggcaggtttggcgaagtcgatgacca | |
| | tcgacacgcgaggaactatgacgaccaagaagcgaaaaaccgccggcgag | |
| | gacctggcaaaacaggtcagcgaggccaagcaggccgcgttgctgaaaca | |
| | cacgaagcagcagatcaaggaaatgcagctttccttgttcgatattgcgc | |
| | cgtggccggacacgatgcgagcgatgccaaacgacacggcccgctctgcc | |
| | ctgttcaccacgcgcaacaagaaaatcccgcgcgaggcgctgcaaaacaa | |
| | ggtcattttccacgtcaacaaggacgtgaagatcacctacaccggcgtcg | |
| | agctgcgggccgacgatgacgaactggtgtggcagcaggtgttggagtac | |
| | gcgaagcgcacccctatcggcgagccgatcaccttcacgttctacgagct | |
| | ttgccaggacctgggctggtcgatcaatggccggtattacacgaaggccg | |
| | aggaatgcctgtcgcgcctacaggcgacggcgatgggcttcacgtccgac | |
| | cgcgttgggcacctggaatcggtgtcgctgctgcaccgcttccgcgtcct | |
| | ggaccgtggcaagaaaacgtcccgttgccaggtcctgatcgacgaggaaa | |
| | tcgtcgtgctgtttgctggcgaccactacacgaaattcatatgggagaag | |
| | taccgcaagctgtcgccgacggcccgacggatgttcgactatttcagctc | |
| | gcaccgggagccgtacccgctcaagctggaaaccttccgcctcatgtgcg | |
| | gatcggattccacccgcgtgaagaagtggcgcgagcaggtcggcgaagcc | |
| | tgcgaagagttgcgaggcagcggcctggtggaacacgcctgggtcaatga | |
| | tgacctggtgcattgcaaacgctagggccttgtggggtcagttccggctg | |
| | ggggttcagcagcccctgctcggatctgttggaccggacagtagtcatgg | |
| | ttgatgggctgcctgtatcgagtggtgattttgtgccgagctgccggtcg | |
| | gggagctgttggctggctggtggcaggatatattgtggtgtaaacaaatt | |
| | gacgcttagacaacttaataacacattgcggacgtttttaatgtactggg | |
| | gttgaacactct | |
| 62 | tgttcaaaaaaaaaagtgattttgagatataatatacatacgacaattaa | PAP3::dC |
| | tacaatattatttttgtgtctatccctctcttagaccctattatttatca | as9-TV |
| | catttgaaattcagaattttctttttctctcgttgttgtacaaatatcta | In plasmid |
| | atcgttaattattattaatgcattaaatatttaaaatataattaatgata | in SEQ ID |
| | ttaattttcctagcgtaagactaataataaatatcgacattatgcgagat | NO: 99. |
| | tatgacatgatgttatgttaatagtacagaataaagtaaaaaaatatata | |
| | taacaataataatttaaaatatttaaaatgacatataaagtagttttcta | |
| | aaacttcagtgattagttcaatttattatttcgcaacaattatgatggtt | |
| | aattaaggtgaattagaagaagaactgaaaattaagataattaaggagaa | |
| | caaatgagcacttcaaaatttgctattaagccgactctgctagccatgca | |
| | tgttggatgtaacttcacccgattgatgggtccttaattataatttttct | |
| | tatgataaaaaataaattgctactaaggcgtactgaactattcgaaatga | |
| | caacactacaaggcctcgacaacgttaacttttttccaatttagtggggc | |
| | gttacaagctaaagcttttttatttttgaactttaaattgctctttcact | |
| | ttcatttaataacgcctaaacctagctacgagcaatataacctgggtgta | |
| | tatagctatatatatcatatgcagagcaatctgtaaccccaaggataaaa | |
| | cgttgtgttgcttattatagaaatcacagttttaaatggaacgagtgaca | |
| | gagtttgtttgtttctaaataggataggaggcatttaatcggaaattgga | |
| | caggagaaagttgttttctttctgcaaattatgcctccagaacttgtttt | |
| | acggtaagaaaatttacaccaaatttacaagtattggttctgaacgctac | |
| | ttgcggaaattttatgccgccagtgacttcttggcactttaaataagtta | |
| | attagttaatcatttgaaaatatattaacaagaagaatttttttctagtg | |
| | taatcaagaggtattttaagcttctatcttacactcaattaatcttctat | |
| | tctcctattgattggtctattttaaagtaaagtaatatattcaattacga | |
| | cttcatgctaaattcaaagattataacagtaaaaacaatttttattcacc | |
| | tctattcaaactgccagtttagcgcaagtgtttatttttctgataaactt | |
| | gcggccgcacaaaacccagcattgcttcaatgtgcataagcatgggtacg | |
| | gtggtggtgtttctccaacctccatttcagattgtcttgatttgtaattc | |
| | aaacacacacttagtcttaaaatcatattttctcatatctcaattgtgat | |
| | ttttccaaatgcgttgtgtgtaggcgaagctaagtttaggttattgctca | |
| | agtgccacaagtttgagagggttttactgtcttttttatttaatctagaa | |
| | atatgatttttggacacgtctacttgttacattccgtttacttatggtag | |
| | tatttgactaataaagacattaagattatagtagcatttgactaataaag | |
| | acacgcgtacgtaggtcactattttattggctaagtaaaaaaaaaaaaaa | |
| | acacgaaaaaactttcaaacaaaggggaagcggagaaatcagcctaactt | |
| | gatattagctgtacaaaatacataaatagctgtcggaaataattatctct | |
| | tgcagtatatatatagaaagtagcagactctaaaatataacatgaagatg | |
| | tgattaagcatttattccattatatggcccataaacatgcatgatgcaaa | |
| | tagaaggatgtagggtaaagttgccctaagaaacgaattgaactgcggtc | |
| | gttgcggtatgagaaagagagagagaaaagtgagtgaaggaaggtgggag | |
| | aatggtttccgtgacggagtaggacggaaagtaagggtccccacagtgca | |
| | catgggagcggatttgacgatgagtcgagaaagagcacaagacgtttatg | |
| | acaaagcctgcaatagctttcgttaatttggcaaaagacccctaccattt | |
| | gcggcaatgcacctccccgttctattatttctctttccctcattcattat | |
| | atcccctattctctgccccagatogatcgaacaagtgaattagctagaag | |
| | cattgaaggacgtagctgctccatggattacaaagatgacgatgataaag | |
| | actacaaggacgacgacgataaaatggcccctaaaaagaagcgcaaggtg | |
| | ggtattcatggggtgcctgcagctggaatggataagaagtactccatcgg | |
| | actggctatcggaactaactccgtgggatgggctgtgatcactgatgagt | |
| | acaaggtgccatccaagaagttcaaggtgctgggaaacactgatagacac | |
| | tccatcaagaagaacctgatcggagctctgctgttcgattccggagagac | |
| | tgctgaggctactagactgaagagaactgctagaagaagatacactagaa | |
| | gaaagaacagaatctgctacctgcaagagatcttctccaacgagatggct | |
| | aaggtggatgattccttcttccacagactggaggagtccttcctggtgga | |
| | ggaggataagaagcacgagagacacccaatcttcggaaacatcgtggatg | |
| | aggtggcttaccacgagaagtacccaactatctaccacctgagaaagaag | |
| | ctggtggattccactgataaggctgatctgagactgatctacctggctct | |
| | ggctcacatgatcaagttcagaggacacttcctgatcgagggagatctga | |
| | acccagataactccgatgtggataagctgttcatccaactggtgcaaact | |
| | tacaaccaactgttcgaggagaacccaatcaacgcttccggagtggatgc | |
| | taaggctatcctgtccgctagactgtccaagtccaggagactggagaacc | |
| | tgatcgctcaactgccaggagagaagaagaacggactgttcggaaacctg | |
| | atcgctctgtccctgggactgactccaaacttcaagtccaacttcgatct | |
| | ggctgaggatgctaagctgcaactgtccaaggatacttacgatgatgatc | |
| | tggataacctgctggctcaaatcggagatcaatacgctgatctgttcctg | |
| | gctgctaagaacctgtccgatgctatcctgctgtccgatatcctgagagt | |
| | gaacactgagatcactaaggctccactgtccgcttccatgatcaagagat | |
| | acgatgagcaccaccaagatctgactctgctgaaggctctggtgagacaa | |
| | caactgccagagaagtacaaggagatcttcttcgatcaatccaagaacgg | |
| | atacgctggatacatcgatggaggagcttcccaagaggagttctacaagt | |
| | tcatcaagccaatcctggagaagatggatggaactgaggagctgctggtg | |
| | aagctgaacagagaggatctgctgagaaagcaaagaactttcgataacgg | |
| | atccatcccacaccaaatccacctgggagagctgcacgctatcctgagga | |
| | gacaagaggatttctacccattcctgaaggataacagagagaagatcgag | |
| | aagatcctgactttcagaatcccatactacgtgggaccactggctagagg | |
| | aaactccagattcgcttggatgactagaaagtccgaggagactatcactc | |
| | catggaacttcgaggaggtaagtttctgcttctacctttgatatatatat | |
| | aataattatcattaattagtagtaatataatatttcaaatatttttttca | |
| | aaataaaagaatgtagtatatagcaattgcttttctgtagtttataagtg | |
| | tgtatattttaatttataacttttctaatatatgaccaaaatttgttgat | |
| | gtgcaggtggtggataagggagcttccgctcaatccttcatcgagagaat | |
| | gactaacttcgataagaacctgccaaacgagaaggtgctgccaaagcact | |
| | ccctgctgtacgagtacttcactgtgtacaacgagctgactaaggtgaag | |
| | tacgtgactgagggaatgagaaagccagctttcctgtccggagagcaaaa | |
| | gaaggctatcgtggatctgctgttcaagactaacagaaaggtgactgtga | |
| | agcaactgaaggaggattacttcaagaagatcgagtgcttcgattccgtg | |
| | gagatctccggagtggaggatagattcaacgcttccctgggaacttacca | |
| | cgatctgctgaagatcatcaaggataaggatttcctggataacgaggaga | |
| | acgaggatatcctggaggatatcgtgctgactctgactctgttcgaggat | |
| | agagagatgatcgaggagagacttaagacttacgctcacctgttcgatga | |
| | taaggtgatgaagcaactgaagagaagaagatacactggatggggtagac | |
| | tgtccagaaagctgatcaacggaatcagagataagcaatccggaaagact | |
| | atcctggatttcctgaagtccgatggattcgctaacagaaacttcatgca | |
| | actgatccacgatgattccctgactttcaaggaggatatccaaaaggctc | |
| | aagtgtccggacaaggagattccctgcacgagcacatcgctaacctggct | |
| | ggatccccagctatcaagaagggaatcctgcaaactgtgaaggtggtgga | |
| | tgagctggtgaaggtgatgggtagacacaagccagagaacatcgtgatcg | |
| | agatggctagagagaaccaaactactcaaaagggacaaaagaactccaga | |
| | gagagaatgaagagaatcgaggagggaatcaaggagctgggatcccaaat | |
| | cctgaaggagcacccagtggagaacactcaactgcaaaacgagaagctgt | |
| | acctgtactacctgcaaaacggaagagatatgtacgtggatcaagagctg | |
| | gatatcaacagactgtccgattacgatgtggatgcaatcgtgccacaatc | |
| | cttcctgaaggatgattccatcgataacaaggtgctgactagatccgata | |
| | agaacagaggaaagtccgataacgtgccatccgaggaggtggtgaagaag | |
| | atgaagaactactggagacaactgctgaacgctaagctgatcactcaaag | |
| | aaagttcgataacctgactaaggctgagagaggaggactgtccgagctgg | |
| | ataaggctggattcatcaagagacaactggtggagactagacaaatcact | |
| | aagcacgtggctcaaatcctggattccagaatgaacactaagtacgatga | |
| | gaacgataagctgatcagagaggtgaaggtgatcactctgaagtccaagc | |
| | tggtgtccgatttcagaaaggatttccaattctacaaggtgagagagatc | |
| | aacaactaccaccacgctcacgatgcttacctgaacgctgtggtgggaac | |
| | tgctctgatcaagaagtacccaaagctggagtccgagttcgtgtacggag | |
| | attacaaggtgtacgatgtgagaaagatgatcgctaagtccgagcaagag | |
| | atcggaaaggctactgctaagtacttcttctactccaacatcatgaactt | |
| | cttcaagactgagatcactctggctaacggagagatcagaaaaagaccac | |
| | tgatcgagactaacggagagactggagagatcgtgtgggataagggaaga | |
| | gatttcgctactgtgagaaaggtgctgtccatgccacaagtgaacatcgt | |
| | gaaaaagactgaggtgcaaactggaggattctccaaggagtccatcctgc | |
| | caaagagaaactccgataagctgatcgctagaaagaaggattgggatcca | |
| | aagaagtacggaggattcgattccccaactgtggcttactccgtgctggt | |
| | ggtggctaaggtggagaagggaaagtccaagaagctgaagtccgtgaagg | |
| | agctgctgggaatcactatcatggagagatcctccttcgagaagaaccca | |
| | atcgatttcctggaggctaagggatacaaggaggtgaagaaggatctgat | |
| | catcaagctgccaaagtactccctgttcgagctggagaacggaagaaaga | |
| | gaatgctggcttccgctggagagctgcaaaagggaaacgagctggctctg | |
| | ccatccaagtacgtgaacttcctgtacctggcttcccactacgagaagct | |
| | gaagggatccccagaggataacgagcaaaagcaactgttcgtggagcaac | |
| | acaagcactacctggatgagatcatcgagcaaatctccgagttctccaag | |
| | agagtgatcctggctgatgctaacctggataaggtgctgtccgcttacaa | |
| | caagcacagagataagccaatcagagagcaagctgagaacatcatccacc | |
| | tgttcactctgactaacctgggagctccagctgctttcaagtacttcgat | |
| | actactatcgatagaaagagatacacttccactaaggaggtgctggatgc | |
| | tactctgatccaccaatccatcactggactgtacgagactagaatcgatc | |
| | tgtcccaactgggaggagatagttcgaagaggcctgccgccacaaagaaa | |
| | gccggccaggcaaagaaaaaaaaacccaggggagggagtggtggactect | |
| | cgaccctggcactccaatggacgccgatctcgtagcatcatctaccgtcg | |
| | tgtgggagcaggacgccgacccttttgctggcacagctgatgatttccca | |
| | gcttttaatgaggaagaattggcttggctgatggagcttctcccacaagg | |
| | tggtagcggcgggctcctcgatcccggtactcccatggacgcagatcttg | |
| | ttgctagctccactgttgtatgggagcaggacgccgatcccttcgctgga | |
| | accgccgatgatttcccagcattcaacgaggaagaacttgcatggctcat | |
| | ggagctcttgccccaagctcgcggtggatctgggggtttgttggatcccg | |
| | gaaccccaatggacgcagacctcgtggcttctagtaccgtagtatgggaa | |
| | caggacgctgaccctttcgccggcaccgcagatgacttcccagcattcaa | |
| | cgaggaggaactggcctggctgatggaacttctgcctcagggggatccgg | |
| | aggcctcttggaccccggtacccctatggacgctgacctggtcgcatctt | |
| | ctaccgtggtttgggagcaagatgccgacccattcgcaggaacagctgat | |
| | gacttccccgcattcaatgaagaagagctcgcatggctgatggaattgct | |
| | tccacaagccagaggcggctctggagggcttcttgatcccggaacaccca | |
| | tggatgccgacctcgtggcctctagcaccgtggtatgggaacaagacgca | |
| | gatccattcgcagggaccgcagacgatttccccgcttttaacgaggagga | |
| | gcttgcttggctcatggagctgctgcctcagggtggatccgggggacttc | |
| | tcgacccaggcacacccatggatgccgatctcgtcgccagttctaccgtt | |
| | gtctgggagcaagacgcagaccctttcgccggaactgcagacgacttccc | |
| | tgcctttaatgaagaagaactggcttggctcatggaactgctcccccagg | |
| | ctagagggggtagcggcgggggcggcagcggaggtgatgcacttgacgac | |
| | ttcgatcttgatatgcttggcagcgacgcacttgatgacttcgatttgga | |
| | tatgctggggtccgatgctcttgacgattttgatttggacatgttgggtt | |
| | cagacgcactcgatgatttcgatttggacatgctggcacgtggaagcgat | |
| | gcccttgatgactttgacctggatatgctcgggtccgatgctttggacga | |
| | cttcgacctcgacatgttgggaagtgatgctcttgatgatttcgatcttg | |
| | atatgcttgggtccgacgctctcgatgattttgacctcgatatgctctga | |
| 63 | atatttgaatatacttaatgttgatgttaaatattatttcatccatctta | pAGL1:: |
| | aaataagtgtcattctatttattttatatagacaaaaaaaagttaataaa | dCas9-TV |
| | ttaataaaaaaacagtaattttataaaactaaccttagaattaatatttc | In plasmid |
| | attaaaaaactaaaattacacttattagaaatattagaaaaaaaatatca | in SEQ ID |
| | ctactaaaaatattattttttatgacgaacattctaaaatggttatatga | NO: 69. |
| | aatcattttagaatgtcatgcgatgacaattgtaaatacaagattgttta | |
| | acaaagacgattttacaaaaattgtcttaaaaattattgttttatgacgg | |
| | tttgatgaaaaccgtcttaaaatgattgtcattctaaaacgattttcgta | |
| | aaatcctcttaaaagttagtgcttttaatttttatgtctccctctcctct | |
| | ctcatttctctccctctcactctctactacttcttccgttccaaaataat | |
| | tgttgtattaggttattttacataggtgaagtaataaatagatgaaatat | |
| | aatgataattttagaaaattaatcttatatcatcatttaatacatttata | |
| | tatagttcatcattaataaataaaaaaataattaatgttacatttaaaaa | |
| | ctaaaatgataaaagtaattaatatacattaagaaactaaaatgacatat | |
| | ttttcttatacgacaattataacacgatgagggagtaatgaatattagtt | |
| | aaaattagtttttgatcacctaatttataattttgattcaatttagtcct | |
| | ttagttattttgggttcaatttaatcctctaatttttaaaattatttttt | |
| | ttactttccattccatcacaagttccaaatgaatttgaatttaatcaact | |
| | tatggtagtatatatagaactttgaaagattattatctattgtaaaaaaa | |
| | ttcaatcttaagattttttttgcataacatgattcctaaactactcatca | |
| | ggtgtcttaaagatcagttactagtgtaatggagctagctacttggctca | |
| | catttcaggtattttacgtaaaagtccttacatgcactgtcgccctaaca | |
| | ctttgtttatgctaaccttgtcatcaaaatcaaaatgcagaaaagggaaa | |
| | agaaagaaaaaattcaaactggctttcccatccccataaagacttgctaa | |
| | atataaggagaagaagtgaagattatgatcagacatcacgtgggaaccct | |
| | tcttagttaaaattgtaataattcaagagagagggtcaagagtcactgga | |
| | aactctcaggtttcaattttcaactgtacaaaataaaaattaaagagatg | |
| | caggggtcagttaaaggaaataaatcttgtagatataaatatctatatat | |
| | agattatggttggataactttttatagaagaagaaaagaaaaacttgtct | |
| | tataaatttaaatcatcttacacatgcatgcacttgaattttatagaaac | |
| | tcttataagctaaatatataaattaattttaatttatgaaaatatatagt | |
| | ttgcttcattttactatttatttacttttgttataagtgtaaacaaggcc | |
| | atattcgaagtaaaaggccaaaaactcaaaatccaagaagcaaaatggtt | |
| | tctttattttttttatgctttaaaaatttgaagcttcggaattttgcgtg | |
| | caaacttttatgtgcatgtccctctcattgaaattgaatcaaaccccaca | |
| | ttaacaaattaaagtcactttcgaaaagaccctttgattatctccctctc | |
| | ctttttoccatacatgaagaaatggatggtttatggagcatgaaaggtga | |
| | acctcaagtctctctctctctcttctctctcatgcttaaattctccaaca | |
| | cgagagtaaattttagagtgagtggtaattgtggagggtctatttttctg | |
| | tcataaatacctgtctgctaggccaacccacttccatattttgcatcctt | |
| | tgtacctcagttccaggttaacaccaaacaacacatatagtgcagattca | |
| | taaggtatagactttgttaatttgcaccctctcttaccctaaatcttgtg | |
| | tctattgtattttatctctttccccatggattacaaagatgacgatgata | |
| | aagactacaaggacgacgacgataaaatggcccctaaaaagaagcgcaag | |
| | gtgggtattcatggggtgcctgcagctggaatggataagaagtactccat | |
| | cggactggctatcggaactaactccgtgggatgggctgtgatcactgatg | |
| | agtacaaggtgccatccaagaagttcaaggtgctgggaaacactgataga | |
| | cactccatcaagaagaacctgatcggagctctgctgttcgattccggaga | |
| | gactgctgaggctactagactgaagagaactgctagaagaagatacacta | |
| | gaagaaagaacagaatctgctacctgcaagagatcttctccaacgagatg | |
| | gctaaggtggatgattccttcttccacagactggaggagtccttcctggt | |
| | ggaggaggataagaagcacgagagacacccaatcttcggaaacatcgtgg | |
| | atgaggtggcttaccacgagaagtacccaactatctaccacctgagaaag | |
| | aagctggtggattccactgataaggctgatctgagactgatctacctggc | |
| | tctggctcacatgatcaagttcagaggacacttcctgatcgagggagatc | |
| | tgaacccagataactccgatgtggataagctgttcatccaactggtgcaa | |
| | acttacaaccaactgttcgaggagaacccaatcaacgcttccggagtgga | |
| | tgctaaggctatcctgtccgctagactgtccaagtccaggagactggaga | |
| | acctgatcgctcaactgccaggagagaagaagaacggactgttcggaaac | |
| | ctgatcgctctgtccctgggactgactccaaacttcaagtccaacttcga | |
| | tctggctgaggatgctaagctgcaactgtccaaggatacttacgatgatg | |
| | atctggataacctgctggctcaaatcggagatcaatacgctgatctgttc | |
| | ctggctgctaagaacctgtccgatgctatcctgctgtccgatatcctgag | |
| | agtgaacactgagatcactaaggctccactgtccgcttccatgatcaaga | |
| | gatacgatgagcaccaccaagatctgactctgctgaaggctctggtgaga | |
| | caacaactgccagagaagtacaaggagatcttcttcgatcaatccaagaa | |
| | cggatacgctggatacatcgatggaggagcttcccaagaggagttctaca | |
| | agttcatcaagccaatcctggagaagatggatggaactgaggagctgctg | |
| | gtgaagctgaacagagaggatctgctgagaaagcaaagaactttcgataa | |
| | cggatccatcccacaccaaatccacctgggagagctgcacgctatcctga | |
| | ggagacaagaggatttctacccattcctgaaggataacagagagaagatc | |
| | gagaagatcctgactttcagaatcccatactacgtgggaccactggctag | |
| | aggaaactccagattcgcttggatgactagaaagtccgaggagactatca | |
| | ctccatggaacttcgaggaggtaagtttctgcttctacctttgatatata | |
| | tataataattatcattaattagtagtaatataatatttcaaatatttttt | |
| | tcaaaataaaagaatgtagtatatagcaattgcttttctgtagtttataa | |
| | gtgtgtatattttaatttataacttttctaatatatgaccaaaatttgtt | |
| | gatgtgcaggtggtggataagggagcttccgctcaatccttcatcgagag | |
| | aatgactaacttcgataagaacctgccaaacgagaaggtgctgccaaagc | |
| | actccctgctgtacgagtacttcactgtgtacaacgagctgactaaggtg | |
| | aagtacgtgactgagggaatgagaaagccagctttcctgtccggagagca | |
| | aaagaaggctatcgtggatctgctgttcaagactaacagaaaggtgactg | |
| | tgaagcaactgaaggaggattacttcaagaagatcgagtgcttcgattcc | |
| | gtggagatctccggagtggaggatagattcaacgcttccctgggaactta | |
| | ccacgatctgctgaagatcatcaaggataaggatttcctggataacgagg | |
| | agaacgaggatatcctggaggatatcgtgctgactctgactctgttcgag | |
| | gatagagagatgatcgaggagagacttaagacttacgctcacctgttcga | |
| | tgataaggtgatgaagcaactgaagagaagaagatacactggatggggta | |
| | gactgtccagaaagctgatcaacggaatcagagataagcaatccggaaag | |
| | actatcctggatttcctgaagtccgatggattcgctaacagaaacttcat | |
| | gcaactgatccacgatgattccctgactttcaaggaggatatccaaaagg | |
| | ctcaagtgtccggacaaggagattccctgcacgagcacatcgctaacctg | |
| | gctggatccccagctatcaagaagggaatcctgcaaactgtgaaggtggt | |
| | ggatgagctggtgaaggtgatgggtagacacaagccagagaacatcgtga | |
| | tcgagatggctagagagaaccaaactactcaaaagggacaaaagaactcc | |
| | agagagagaatgaagagaatcgaggagggaatcaaggagctgggatccca | |
| | aatcctgaaggagcacccagtggagaacactcaactgcaaaacgagaagc | |
| | tgtacctgtactacctgcaaaacggaagagatatgtacgtggatcaagag | |
| | ctggatatcaacagactgtccgattacgatgtggatgcaatcgtgccaca | |
| | atccttcctgaaggatgattccatcgataacaaggtgctgactagatccg | |
| | ataagaacagaggaaagtccgataacgtgccatccgaggaggtggtgaag | |
| | aagatgaagaactactggagacaactgctgaacgctaagctgatcactca | |
| | aagaaagttcgataacctgactaaggctgagagaggaggactgtccgagc | |
| | tggataaggctggattcatcaagagacaactggtggagactagacaaatc | |
| | actaagcacgtggctcaaatcctggattccagaatgaacactaagtacga | |
| | tgagaacgataagctgatcagagaggtgaaggtgatcactctgaagtcca | |
| | agctggtgtccgatttcagaaaggatttccaattctacaaggtgagagag | |
| | atcaacaactaccaccacgctcacgatgcttacctgaacgctgtggtggg | |
| | aactgctctgatcaagaagtacccaaagctggagtccgagttcgtgtacg | |
| | gagattacaaggtgtacgatgtgagaaagatgatcgctaagtccgagcaa | |
| | gagatcggaaaggctactgctaagtacttcttctactccaacatcatgaa | |
| | cttcttcaagactgagatcactctggctaacggagagatcagaaaaagac | |
| | cactgatcgagactaacggagagactggagagatcgtgtgggataaggga | |
| | agagatttcgctactgtgagaaaggtgctgtccatgccacaagtgaacat | |
| | cgtgaaaaagactgaggtgcaaactggaggattctccaaggagtccatcc | |
| | tgccaaagagaaactccgataagctgatcgctagaaagaaggattgggat | |
| | ccaaagaagtacggaggattcgattccccaactgtggcttactccgtgct | |
| | ggtggtggctaaggtggagaagggaaagtccaagaagctgaagtccgtga | |
| | aggagctgctgggaatcactatcatggagagatcctccttcgagaagaac | |
| | ccaatcgatttcctggaggctaagggatacaaggaggtgaagaaggatct | |
| | gatcatcaagctgccaaagtactccctgttcgagctggagaacggaagaa | |
| | agagaatgctggcttccgctggagagctgcaaaagggaaacgagctggct | |
| | ctgccatccaagtacgtgaacttcctgtacctggcttcccactacgagaa | |
| | gctgaagggatccccagaggataacgagcaaaagcaactgttcgtggagc | |
| | aacacaagcactacctggatgagatcatcgagcaaatctccgagttctcc | |
| | aagagagtgatcctggctgatgctaacctggataaggtgctgtccgctta | |
| | caacaagcacagagataagccaatcagagagcaagctgagaacatcatcc | |
| | acctgttcactctgactaacctgggagctccagctgctttcaagtacttc | |
| | gatactactatcgatagaaagagatacacttccactaaggaggtgctgga | |
| | tgctactctgatccaccaatccatcactggactgtacgagactagaatcg | |
| | atctgtcccaactgggaggagatagttcgaagaggcctgccgccacaaag | |
| | aaagccggccaggcaaagaaaaaaaaacccaggggagggagtggtggact | |
| | cctcgaccctggcactccaatggacgccgatctcgtagcatcatctaccg | |
| | tcgtgtgggagcaggacgccgacccttttgctggcacagctgatgatttc | |
| | ccagcttttaatgaggaagaattggcttggctgatggagcttctcccaca | |
| | aggtggtagcggcgggctcctcgatcccggtactcccatggacgcagatc | |
| | ttgttgctagctccactgttgtatgggagcaggacgccgatcccttcgct | |
| | ggaaccgccgatgatttcccagcattcaacgaggaagaacttgcatggct | |
| | catggagctcttgccccaagctcgcggtggatctgggggtttgttggatc | |
| | ccggaaccccaatggacgcagacctcgtggcttctagtaccgtagtatgg | |
| | gaacaggacgctgaccctttcgccggcaccgcagatgacttcccagcatt | |
| | caacgaggaggaactggcctggctgatggaacttctgcctcagggtggat | |
| | ccggaggcctcttggaccccggtacccctatggacgctgacctggtcgca | |
| | tcttctaccgtggtttgggagcaagatgccgacccattcgcaggaacagc | |
| | tgatgacttccccgcattcaatgaagaagagctcgcatggctgatggaat | |
| | tgcttccacaagccagaggeggctctggagggcttcttgatcccggaaca | |
| | cccatggatgccgacctcgtggcctctagcaccgtggtatgggaacaaga | |
| | cgcagatccattcgcagggaccgcagacgatttccccgcttttaacgagg | |
| | aggagcttgcttggctcatggagctgctgcctcagggtggatccggggga | |
| | cttctcgacccaggcacacccatggatgccgatctcgtcgccagttctac | |
| | cgttgtctgggagcaagacgcagaccctttcgccggaactgcagacgact | |
| | tccctgcctttaatgaagaagaactggcttggctcatggaactgctcccc | |
| | caggctagagggggtagcggcgggggcggcagcggaggtgatgcacttga | |
| | cgacttcgatcttgatatgcttggcagcgacgcacttgatgacttcgatt | |
| | tggatatgctggggtccgatgctcttgacgattttgatttggacatgttg | |
| | ggttcagacgcactcgatgatttcgatttggacatgctggcacgtggaag | |
| | cgatgcccttgatgactttgacctggatatgctcgggtccgatgctttgg | |
| | acgacttcgacctcgacatgttgggaagtgatgctcttgatgatttcgat | |
| | cttgatatgcttgggtccgacgctctcgatgattttgacctcgatatgct | |
| | ctga | |
| 64 | tgttcaaaaataaaaagtgattttgagatataatatacatacgacaatta | pAP3::dC |
| | atacaatattatttttgtgtctatccctctcttagaccctattatttatc | as9-SRDX |
| | acatttgaaattcagaattttctttttctctcgttgttgtacaaatatct | |
| | aatcgttaattattattaatgcattaaatatttaaaatataattaatgat | |
| | attaattttcctagcgtaagactaataataaatatcgacattatgcgaga | |
| | ttatgacatgatgttatgttaatagtacagaataaagtaaaaaaatatat | |
| | ataacaataataatttaaaatatttaaaatgacatataaagtagttttct | |
| | aaaacttcagtgattagttcaatttattatttcgcaacaattatgatggt | |
| | taattaaggtgaattagaagaagaactgaaaattaagataattaaggaga | |
| | acaaatgagcacttcaaaatttgctattaagccgactctgctagccatgc | |
| | atgttggatgtaacttcacccgattgatgggtccttaattataatttttc | |
| | ttatgataaaaaataaattgctactaaggcgtactgaactattcgaaatg | |
| | acaacactacaaggcctcgacaacgttaacttttttccaatttagtgggg | |
| | cgttacaagctaaagcttttttatttttgaactttaaattgctctttcac | |
| | tttcatttaataacgcctaaacctagctacgagcaatataacctgggtgt | |
| | atatagctatatatatcatatgcagagcaatctgtaaccccaaggataaa | |
| | acgttgtgttgcttattatagaaatcacagttttaaatggaacgagtgac | |
| | agagtttgtttgtttctaaataggataggaggcatttaatcggaaattgg | |
| | acaggagaaagttgttttctttctgcaaattatgcctccagaacttgttt | |
| | tacggtaagaaaatttacaccaaatttacaagtattggttctgaacgcta | |
| | cttgcggaaattttatgccgccagtgacttcttggcactttaaataagtt | |
| | aattagttaatcatttgaaaatatattaacaagaagaatttttttctagt | |
| | gtaatcaagaggtattttaagcttctatcttacactcaattaatcttcta | |
| | ttctcctattgattggtctattttaaagtaaagtaatatattcaattacg | |
| | acttcatgctaaattcaaagattataacagtaaaaacaatttttattcac | |
| | ctctattcaaactgccagtttagcgcaagtgtttatttttctgataaact | |
| | tgcggccgcacaaaacccagcattgcttcaatgtgcataagcatgggtac | |
| | ggtggtggtgtttctccaacctccatttcagattgtcttgatttgtaatt | |
| | caaacacacacttagtcttaaaatcatattttctcatatctcaattgtga | |
| | tttttccaaatgcgttgtgtgtaggcgaagctaagtttaggttattgctc | |
| | aagtgccacaagtttgagagggttttactgtcttttttatttaatctaga | |
| | aatatgatttttggacacgtctacttgttacattccgtttacttatggta | |
| | gtatttgactaataaagacattaagattatagtagcatttgactaataaa | |
| | gacacgcgtacgtaggtcactattttattggctaagtaaaaaaaaaaaaa | |
| | aacacgaaaaaactttcaaacaaaggggaagcggagaaatcagcctaact | |
| | tgatattagctgtacaaaatacataaatagctgtcggaaataattatctc | |
| | ttgcagtatatatatagaaagtagcagactctaaaatataacatgaagat | |
| | gtgattaagcatttattccattatatggcccataaacatgcatgatgcaa | |
| | atagaaggatgtagggtaaagttgccctaagaaacgaattgaactgcggt | |
| | cgttgcggtatgagaaagagagagagaaaagtgagtgaaggaaggtggga | |
| | gaatggtttccgtgacggagtaggacggaaagtaagggtccccacagtgc | |
| | acatgggagcggatttgacgatgagtcgagaaagagcacaagacgtttat | |
| | gacaaagcctgcaatagctttcgttaatttggcaaaagacccctaccatt | |
| | tgcggcaatgcacctccccgttctattatttctctttccctcattcatta | |
| | tatcccctattctctgccccagatcgatcgaacaagtgaattagctagaa | |
| | gcattgaaggacgtagctgctccatggattacaaagatgacgatgataaa | |
| | gactacaaggacgacgacgataaaatggcccctaaaaagaagcgcaaggt | |
| | gggtattcatggggtgcctgcagctggaatggataagaagtactccatcg | |
| | gactggctatcggaactaactccgtgggatgggctgtgatcactgatgag | |
| | tacaaggtgccatccaagaagttcaaggtgctgggaaacactgatagaca | |
| | ctccatcaagaagaacctgatcggagctctgctgttcgattccggagaga | |
| | ctgctgaggctactagactgaagagaactgctagaagaagatacactaga | |
| | agaaagaacagaatctgctacctgcaagagatcttctccaacgagatggc | |
| | taaggtggatgattccttcttccacagactggaggagtccttcctggtgg | |
| | aggaggataagaagcacgagagacacccaatcttcggaaacatcgtggat | |
| | gaggtggcttaccacgagaagtacccaactatctaccacctgagaaagaa | |
| | gctggtggattccactgataaggctgatctgagactgatctacctggctc | |
| | tggctcacatgatcaagttcagaggacacttcctgatcgagggagatctg | |
| | aacccagataactccgatgtggataagctgttcatccaactggtgcaaac | |
| | ttacaaccaactgttcgaggagaacccaatcaacgcttccggagtggatg | |
| | ctaaggctatcctgtccgctagactgtccaagtccaggagactggagaac | |
| | ctgatcgctcaactgccaggagagaagaagaacggactgttcggaaacct | |
| | gatcgctctgtccctgggactgactccaaacttcaagtccaacttcgatc | |
| | tggctgaggatgctaagctgcaactgtccaaggatacttacgatgatgat | |
| | ctggataacctgctggctcaaatcggagatcaatacgctgatctgttcct | |
| | ggctgctaagaacctgtccgatgctatcctgctgtccgatatcctgagag | |
| | tgaacactgagatcactaaggctccactgtccgcttccatgatcaagaga | |
| | tacgatgagcaccaccaagatctgactctgctgaaggctctggtgagaca | |
| | acaactgccagagaagtacaaggagatcttcttcgatcaatccaagaacg | |
| | gatacgctggatacatcgatggaggagcttcccaagaggagttctacaag | |
| | ttcatcaagccaatcctggagaagatggatggaactgaggagctgctggt | |
| | gaagctgaacagagaggatctgctgagaaagcaaagaactttcgataacg | |
| | gatccatcccacaccaaatccacctgggagagctgcacgctatcctgagg | |
| | agacaagaggatttctacccattcctgaaggataacagagagaagatcga | |
| | gaagatcctgactttcagaatcccatactacgtgggaccactggctagag | |
| | gaaactccagattcgcttggatgactagaaagtccgaggagactatcact | |
| | ccatggaacttcgaggaggtaagtttctgcttctacctttgatatatata | |
| | taataattatcattaattagtagtaatataatatttcaaatatttttttc | |
| | aaaataaaagaatgtagtatatagcaattgcttttctgtagtttataagt | |
| | gtgtatattttaatttataacttttctaatatatgaccaaaatttgttga | |
| | tgtgcaggtggtggataagggagcttccgctcaatccttcatcgagagaa | |
| | tgactaacttcgataagaacctgccaaacgagaaggtgctgccaaagcac | |
| | tccctgctgtacgagtacttcactgtgtacaacgagctgactaaggtgaa | |
| | gtacgtgactgagggaatgagaaagccagctttcctgtccggagagcaaa | |
| | agaaggctatcgtggatctgctgttcaagactaacagaaaggtgactgtg | |
| | aagcaactgaaggaggattacttcaagaagatcgagtgcttcgattccgt | |
| | ggagatctccggagtggaggatagattcaacgcttccctgggaacttacc | |
| | acgatctgctgaagatcatcaaggataaggatttcctggataacgaggag | |
| | aacgaggatatcctggaggatatcgtgctgactctgactctgttcgagga | |
| | tagagagatgatcgaggagagacttaagacttacgctcacctgttcgatg | |
| | ataaggtgatgaagcaactgaagagaagaagatacactggatggggtaga | |
| | ctgtccagaaagctgatcaacggaatcagagataagcaatccggaaagac | |
| | tatcctggatttcctgaagtccgatggattcgctaacagaaacttcatgc | |
| | aactgatccacgatgattccctgactttcaaggaggatatccaaaaggct | |
| | caagtgtccggacaaggagattccctgcacgagcacatcgctaacctggc | |
| | tggatccccagctatcaagaagggaatcctgcaaactgtgaaggtggtgg | |
| | atgagctggtgaaggtgatgggtagacacaagccagagaacatcgtgatc | |
| | gagatggctagagagaaccaaactactcaaaagggacaaaagaactccag | |
| | agagagaatgaagagaatcgaggagggaatcaaggagctgggatcccaaa | |
| | tcctgaaggagcacccagtggagaacactcaactgcaaaacgagaagctg | |
| | tacctgtactacctgcaaaacggaagagatatgtacgtggatcaagagct | |
| | ggatatcaacagactgtccgattacgatgtggatgcaatcgtgccacaat | |
| | ccttcctgaaggatgattccatcgataacaaggtgctgactagatccgat | |
| | aagaacagaggaaagtccgataacgtgccatccgaggaggtggtgaagaa | |
| | gatgaagaactactggagacaactgctgaacgctaagctgatcactcaaa | |
| | gaaagttcgataacctgactaaggctgagagaggaggactgtccgagctg | |
| | gataaggctggattcatcaagagacaactggtggagactagacaaatcac | |
| | taagcacgtggctcaaatcctggattccagaatgaacactaagtacgatg | |
| | agaacgataagctgatcagagaggtgaaggtgatcactctgaagtccaag | |
| | ctggtgtccgatttcagaaaggatttccaattctacaaggtgagagagat | |
| | caacaactaccaccacgctcacgatgcttacctgaacgctgtggtgggaa | |
| | ctgctctgatcaagaagtacccaaagctggagtccgagttcgtgtacgga | |
| | gattacaaggtgtacgatgtgagaaagatgatcgctaagtccgagcaaga | |
| | gatcggaaaggctactgctaagtacttcttctactccaacatcatgaact | |
| | tcttcaagactgagatcactctggctaacggagagatcagaaaaagacca | |
| | ctgatcgagactaacggagagactggagagatcgtgtgggataagggaag | |
| | agatttcgctactgtgagaaaggtgctgtccatgccacaagtgaacatcg | |
| | tgaaaaagactgaggtgcaaactggaggattctccaaggagtccatcctg | |
| | ccaaagagaaactccgataagctgatcgctagaaagaaggattgggatcc | |
| | aaagaagtacggaggattcgattccccaactgtggcttactccgtgctgg | |
| | tggtggctaaggtggagaagggaaagtccaagaagctgaagtccgtgaag | |
| | gagctgctgggaatcactatcatggagagatcctccttcgagaagaaccc | |
| | aatcgatttcctggaggctaagggatacaaggaggtgaagaaggatctga | |
| | tcatcaagctgccaaagtactccctgttcgagctggagaacggaagaaag | |
| | agaatgctggcttccgctggagagctgcaaaagggaaacgagctggctct | |
| | gccatccaagtacgtgaacttcctgtacctggcttcccactacgagaagc | |
| | tgaagggatccccagaggataacgagcaaaagcaactgttcgtggagcaa | |
| | cacaagcactacctggatgagatcatcgagcaaatctccgagttctccaa | |
| | gagagtgatcctggctgatgctaacctggataaggtgctgtccgcttaca | |
| | acaagcacagagataagccaatcagagagcaagctgagaacatcatccac | |
| | ctgttcactctgactaacctgggagctccagctgctttcaagtacttcga | |
| | tactactatcgatagaaagagatacacttccactaaggaggtgctggatg | |
| | ctactctgatccaccaatccatcactggactgtacgagactagaatcgat | |
| | ctgtcccaactgggaggagatagttcgaagcgcccagccgctaccaaaaa | |
| | agctggacaggctaaaaaaaagaagggtgacggcagcgggtccggctccg | |
| | ggtccggttccgtcgatctcgatttggatctggaacttcgcctggggttc | |
| | gcactcgatctcgaccttgaactgcgtcttggctttgctctcgatcttga | |
| | tctggagttgaggcttggattcgct | |
| 65 | caaagcaccagtggtctagtggtagaatagtaccctgccacggtacagac | gRNAs |
| | ccgggttcgattcccggctggtgcaaaaaaatgtgaatgtggatggtttt | 1a-3a for |
| | agagctagaaatagcaagttaaaataaggctagtccgttatcaacttgaa | ANTa |
| | aaagtggcaccgagtcggtgcaacaaagcaccagtggtctagtggtagaa | Same as |
| | tagtaccctgccacggtacagacccgggttcgattcccggctggtgcaaa | SEQ ID |
| | atttataacatttacatcgttttagagctagaaatagcaagttaaaataa | NO: 75 |
| | ggctagtccgttatcaacttgaaaaagtggcaccgagtcggtgcaacaaa | which has |
| | gcaccagtggtctagtggtagaatagtaccctgccacggtacagacccgg | no |
| | gttcgattcccggctggtgcatgatgtctttagaccagacagttttagag | promoter |
| | ctagaaatagcaagttaaaataaggctagtccgttatcaacttgaaaaag | |
| | tggcaccgagtcggtgccaacaaagcaccagtggtctagtggtagaatag | |
| | taccctgccacggtacagacccgggttcgattcccggctggtgca | |
| 66 | ggattacaaagatgacgatgataaagactacaaggacgacgacgataaaa | DNA |
| | tggcccctaaaaagaagcgcaaggtgggtattcatggggtgcctgcagct | sequence |
| | ggaatggataagaagtactccatcggactggctatcggaactaactccgt | of |
| | gggatgggctgtgatcactgatgagtacaaggtgccatccaagaagttca | 2Xflag::dC |
| | aggtgctgggaaacactgatagacactccatcaagaagaacctgatcgga | as9-TV |
| | gctctgctgttcgattccggagagactgctgaggctactagactgaagag | |
| | aactgctagaagaagatacactagaagaaagaacagaatctgctacctgc | |
| | aagagatcttctccaacgagatggctaaggtggatgattccttcttccac | |
| | agactggaggagtccttcctggtggaggaggataagaagcacgagagaca | |
| | cccaatcttcggaaacatcgtggatgaggtggcttaccacgagaagtacc | |
| | caactatctaccacctgagaaagaagctggtggattccactgataaggct | |
| | gatctgagactgatctacctggctctggctcacatgatcaagttcagagg | |
| | acacttcctgatcgagggagatctgaacccagataactccgatgtggata | |
| | agctgttcatccaactggtgcaaacttacaaccaactgttcgaggagaac | |
| | ccaatcaacgcttccggagtggatgctaaggctatcctgtccgctagact | |
| | gtccaagtccaggagactggagaacctgatcgctcaactgccaggagaga | |
| | agaagaacggactgttcggaaacctgatcgctctgtccctgggactgact | |
| | ccaaacttcaagtccaacttcgatctggctgaggatgctaagctgcaact | |
| | gtccaaggatacttacgatgatgatctggataacctgctggctcaaatcg | |
| | gagatcaatacgctgatctgttcctggctgctaagaacctgtccgatgct | |
| | atcctgctgtccgatatcctgagagtgaacactgagatcactaaggctcc | |
| | actgtccgcttccatgatcaagagatacgatgagcaccaccaagatctga | |
| | ctctgctgaaggctctggtgagacaacaactgccagagaagtacaaggag | |
| | atcttcttcgatcaatccaagaacggatacgctggatacatcgatggagg | |
| | agcttcccaagaggagttctacaagttcatcaagccaatcctggagaaga | |
| | tggatggaactgaggagctgctggtgaagctgaacagagaggatctgctg | |
| | agaaagcaaagaactttcgataacggatccatcccacaccaaatccacct | |
| | gggagagctgcacgctatcctgaggagacaagaggatttctacccattcc | |
| | tgaaggataacagagagaagatcgagaagatcctgactttcagaatccca | |
| | tactacgtgggaccactggctagaggaaactccagattcgcttggatgac | |
| | tagaaagtccgaggagactatcactccatggaacttcgaggaggtaagtt | |
| | tctgcttctacctttgatatatatataataattatcattaattagtagta | |
| | atataatatttcaaatatttttttcaaaataaaagaatgtagtatatagc | |
| | aattgcttttctgtagtttataagtgtgtatattttaatttataactttt | |
| | ctaatatatgaccaaaatttgttgatgtgcaggtggtggataagggagct | |
| | tccgctcaatccttcatcgagagaatgactaacttcgataagaacctgcc | |
| | aaacgagaaggtgctgccaaagcactccctgctgtacgagtacttcactg | |
| | tgtacaacgagctgactaaggtgaagtacgtgactgagggaatgagaaag | |
| | ccagctttcctgtccggagagcaaaagaaggctatcgtggatctgctgtt | |
| | caagactaacagaaaggtgactgtgaagcaactgaaggaggattacttca | |
| | agaagatcgagtgcttcgattccgtggagatctccggagtggaggataga | |
| | ttcaacgcttccctgggaacttaccacgatctgctgaagatcatcaagga | |
| | taaggatttcctggataacgaggagaacgaggatatcctggaggatatcg | |
| | tgctgactctgactctgttcgaggatagagagatgatcgaggagagactt | |
| | aagacttacgctcacctgttcgatgataaggtgatgaagcaactgaagag | |
| | aagaagatacactggatggggtagactgtccagaaagctgatcaacggaa | |
| | tcagagataagcaatccggaaagactatcctggatttcctgaagtccgat | |
| | ggattcgctaacagaaacttcatgcaactgatccacgatgattccctgac | |
| | tttcaaggaggatatccaaaaggctcaagtgtccggacaaggagattccc | |
| | tgcacgagcacatcgctaacctggctggatccccagctatcaagaaggga | |
| | atcctgcaaactgtgaaggtggtggatgagctggtgaaggtgatgggtag | |
| | acacaagccagagaacatcgtgatcgagatggctagagagaaccaaacta | |
| | ctcaaaagggacaaaagaactccagagagagaatgaagagaatcgaggag | |
| | ggaatcaaggagctgggatcccaaatcctgaaggagcacccagtggagaa | |
| | cactcaactgcaaaacgagaagctgtacctgtactacctgcaaaacggaa | |
| | gagatatgtacgtggatcaagagctggatatcaacagactgtccgattac | |
| | gatgtggatgcaatcgtgccacaatccttcctgaaggatgattccatoga | |
| | taacaaggtgctgactagatccgataagaacagaggaaagtccgataacg | |
| | tgccatccgaggaggtggtgaagaagatgaagaactactggagacaactg | |
| | ctgaacgctaagctgatcactcaaagaaagttcgataacctgactaaggc | |
| | tgagagaggaggactgtccgagctggataaggctggattcatcaagagac | |
| | aactggtggagactagacaaatcactaagcacgtggctcaaatcctggat | |
| | tccagaatgaacactaagtacgatgagaacgataagctgatcagagaggt | |
| | gaaggtgatcactctgaagtccaagctggtgtccgatttcagaaaggatt | |
| | tccaattctacaaggtgagagagatcaacaactaccaccacgctcacgat | |
| | gcttacctgaacgctgtggtgggaactgctctgatcaagaagtacccaaa | |
| | gctggagtccgagttcgtgtacggagattacaaggtgtacgatgtgagaa | |
| | agatgatcgctaagtccgagcaagagatcggaaaggctactgctaagtac | |
| | ttcttctactccaacatcatgaacttcttcaagactgagatcactctggc | |
| | taacggagagatcagaaaaagaccactgatcgagactaacggagagactg | |
| | gagagatcgtgtgggataagggaagagatttcgctactgtgagaaaggtg | |
| | ctgtccatgccacaagtgaacatcgtgaaaaagactgaggtgcaaactgg | |
| | aggattctccaaggagtccatcctgccaaagagaaactccgataagctga | |
| | tcgctagaaagaaggattgggatccaaagaagtacggaggattcgattcc | |
| | ccaactgtggcttactccgtgctggtggtggctaaggtggagaagggaaa | |
| | gtccaagaagctgaagtccgtgaaggagctgctgggaatcactatcatgg | |
| | agagatcctccttcgagaagaacccaatcgatttcctggaggctaaggga | |
| | tacaaggaggtgaagaaggatctgatcatcaagctgccaaagtactccct | |
| | gttcgagctggagaacggaagaaagagaatgctggcttccgctggagagc | |
| | tgcaaaagggaaacgagctggctctgccatccaagtacgtgaacttcctg | |
| | tacctggcttcccactacgagaagctgaagggatccccagaggataacga | |
| | gcaaaagcaactgttcgtggagcaacacaagcactacctggatgagatca | |
| | tcgagcaaatctccgagttctccaagagagtgatcctggctgatgctaac | |
| | ctggataaggtgctgtccgcttacaacaagcacagagataagccaatcag | |
| | agagcaagctgagaacatcatccacctgttcactctgactaacctgggag | |
| | ctccagctgctttcaagtacttcgatactactatcgatagaaagagatac | |
| | acttccactaaggaggtgctggatgctactctgatccaccaatccatcac | |
| | tggactgtacgagactagaatcgatctgtcccaactgggaggagatagtt | |
| | cgaagaggcctgccgccacaaagaaagccggccaggcaaagaaaaaaaaa | |
| | cccaggggagggagtggtggactcctcgaccctggcactccaatggacgc | |
| | cgatctcgtagcatcatctaccgtcgtgtgggagcaggacgccgaccctt | |
| | ttgctggcacagctgatgatttcccagcttttaatgaggaagaattggct | |
| | tggctgatggagcttctcccacaaggtggtagegggggctcctcgatccc | |
| | ggtactcccatggacgcagatcttgttgctagctccactgttgtatggga | |
| | gcaggacgccgatcccttcgctggaaccgccgatgatttcccagcattca | |
| | acgaggaagaacttgcatggctcatggagctcttgccccaagctcgcggt | |
| | ggatctgggggtttgttggatcccggaaccccaatggacgcagacctcgt | |
| | ggcttctagtaccgtagtatgggaacaggacgctgaccctttcgccggca | |
| | ccgcagatgacttcccagcattcaacgaggaggaactggcctggctgatg | |
| | gaacttctgcctcagggtggatccggaggcctcttggaccccggtacccc | |
| | tatggacgctgacctggtcgcatcttctaccgtggtttgggagcaagatg | |
| | ccgacccattcgcaggaacagctgatgacttccccgcattcaatgaagaa | |
| | gagctcgcatggctgatggaattgcttccacaagccagaggcggctctgg | |
| | agggcttcttgatcccggaacacccatggatgccgacctcgtggcctcta | |
| | gcaccgtggtatgggaacaagacgcagatccattcgcagggaccgcagac | |
| | gatttccccgcttttaacgaggaggagcttgcttggctcatggagctgct | |
| | gcctcagggtggatccgggggacttctcgacccaggcacacccatggatg | |
| | ccgatctcgtcgccagttctaccgttgtctgggagcaagacgcagaccct | |
| | ttcgccggaactgcagacgacttccctgcctttaatgaagaagaactggc | |
| | ttggctcatggaactgctcccccaggctagagggggtagcggcgggggcg | |
| | gcagcggaggtgatgcacttgacgacttcgatcttgatatgcttggcagc | |
| | gacgcacttgatgacttcgatttggatatgctggggtccgatgctcttga | |
| | cgattttgatttggacatgttgggttcagacgcactcgatgatttcgatt | |
| | tggacatgctggcacgtggaagcgatgcccttgatgactttgacctggat | |
| | atgctcgggtccgatgctttggacgacttcgacctcgacatgttgggaag | |
| | tgatgctcttgatgatttcgatcttgatatgcttgggtccgacgctctcg | |
| | atgattttgacctcgatatgctctga | |
| 67 | gacgtcgttgtggttggtgctttccttacattctgagcctctttccttct | pU10::gR |
| | aatccactcatctgcatcttcttgtgtccttactaatacctcattggttc | NAs 1b-3b |
| | caaattccctccctttaagcaccagctcgtttctgttcttccacagcctc | for ANTb |
| | ccaagtatccaagggactaaagcctccacattcttcagatcaggatattc | Same as |
| | ttgtttaagatgttgaactctatggaggtttgtatgaactgatgatctag | SEQ ID |
| | gaccggataagttcccttcttcatagcgaacttattcaaagaatgttttg | NO: 76 |
| | tgtatcattcttgttacattgttattaatgaaaaaatattattggtcatt | which has |
| | ggactgaacacgagtgttaaatatggaccaggccccaaataagatccatt | no |
| | gatatatgaattaaataacaagaataaatcgagtcaccaaaccacttgcc | promoter |
| | ttttttaacgagacttgttcaccaacttgatacaaaagtcattatcctat | |
| | gcaaatcaataatcatacaaaaatatccaataacactaaaaaaattaaaa | |
| | gaaatggataatttcacaatatgttatacgataaagaagttacttttcca | |
| | agaaattcactgattttataagcccacttgcattagataaatggcaaaaa | |
| | aaaacaaaaaggaaaagaaataaagcacgaagaattctagaaaatacgaa | |
| | atacgcttcaatgcagtgggacccacggttcaattattgccaattttcag | |
| | ctccaccgtatatttaaaaaataaaacgataatgctaaaaaaatataaat | |
| | cgtaacgatcgttaaatctcaacggctggatcttatgacgaccgttaagg | |
| | aaattgtggttgtcggacgaagtccagtaataaacggcgtcaaagtggtt | |
| | gcagccggcacacacgagtcgtgtttatcaactcaaagcacaaatacttt | |
| | tcctcaacctaaaaataaggcaattagccaaaaacaactttgcgtgtaaa | |
| | caacgctcaatacacgtgtcattttattattagctattgcttcaccgcct | |
| | tagctttctcgtgacctagtcgtcctcgtcttttcttcttcttcttctat | |
| | aaaacaatacccaaagagctcttcttcttcacaattcagatttcaatttc | |
| | tcaaaatcttaaaaactttctctcaattctctctaccgtgatcaaggtaa | |
| | atttctgtgttccttattctctcaaaatcttcgattttgttttcgttcga | |
| | tcccaatttcgtatatgttctttggtttagattctgttaatcttagatcg | |
| | tagacgattttctgggtttgatcgttagatatcatcttaattctcgatta | |
| | gggtttcatagatatcatccgatttgttcaaataatttgagttttgtcga | |
| | ataattactcttcgatttgtgatttctatctagatctggtgttagtttct | |
| | agtttgtgcgatcgaatttgtcgattaatctgagtttttctgattaacag | |
| | atgcagatcttaatgacaaagcaccagtggtctagtggtagaatagtacc | |
| | ctgccacggtacagacccgggttcgattcccggctggtgcattttgtatc | |
| | tctctatatctgttttagagctagaaatagcaagttaaaataaggctagt | |
| | ccgttatcaacttgaaaaagtggcaccgagtcggtgcaacaaagcaccag | |
| | tggtctagtggtagaatagtaccctgccacggtacagacccgggttcgat | |
| | tcccggctggtgcatttctttgtgtctcagctcagttttagagctagaaa | |
| | tagcaagttaaaataaggctagtccgttatcaacttgaaaaagtggcacc | |
| | gagtcggtgcaacaaagcaccagtggtctagtggtagaatagtaccctgc | |
| | cacggtacagacccgggttcgattcccggctggtgcatgggaaaagattc | |
| | agaactcgttttagagctagaaatagcaagttaaaataaggctagtccgt | |
| | tatcaacttgaaaaagtggcaccgagtcggtgccaacaaagcaccagtgg | |
| | tctagtggtagaatagtaccctgccacggtacagacccgggttcgattcc | |
| | cggctggtgca | |
| 68 | ggattacaaagatgacgatgataaagactacaaggacgacgacgataaaa | 2Xflag::dC |
| | tggcccctaaaaagaagcgcaaggtgggtattcatggggtgcctgcagct | as9-SRDX |
| | ggaatggataagaagtactccatcggactggctatcggaactaactccgt | DNA |
| | gggatgggctgtgatcactgatgagtacaaggtgccatccaagaagttca | |
| | aggtgctgggaaacactgatagacactccatcaagaagaacctgatcgga | |
| | gctctgctgttcgattccggagagactgctgaggctactagactgaagag | |
| | aactgctagaagaagatacactagaagaaagaacagaatctgctacctgc | |
| | aagagatcttctccaacgagatggctaaggtggatgattccttcttccac | |
| | agactggaggagtccttcctggtggaggaggataagaagcacgagagaca | |
| | cccaatcttcggaaacatcgtggatgaggtggcttaccacgagaagtacc | |
| | caactatctaccacctgagaaagaagctggtggattccactgataaggct | |
| | gatctgagactgatctacctggctctggctcacatgatcaagttcagagg | |
| | acacttcctgatcgagggagatctgaacccagataactccgatgtggata | |
| | agctgttcatccaactggtgcaaacttacaaccaactgttcgaggagaac | |
| | ccaatcaacgcttccggagtggatgctaaggctatcctgtccgctagact | |
| | gtccaagtccaggagactggagaacctgatcgctcaactgccaggagaga | |
| | agaagaacggactgttcggaaacctgatcgctctgtccctgggactgact | |
| | ccaaacttcaagtccaacttcgatctggctgaggatgctaagctgcaact | |
| | gtccaaggatacttacgatgatgatctggataacctgctggctcaaatcg | |
| | gagatcaatacgctgatctgttcctggctgctaagaacctgtccgatgct | |
| | atcctgctgtccgatatcctgagagtgaacactgagatcactaaggctcc | |
| | actgtccgcttccatgatcaagagatacgatgagcaccaccaagatctga | |
| | ctctgctgaaggctctggtgagacaacaactgccagagaagtacaaggag | |
| | atcttcttcgatcaatccaagaacggatacgctggatacatcgatggagg | |
| | agcttcccaagaggagttctacaagttcatcaagccaatcctggagaaga | |
| | tggatggaactgaggagctgctggtgaagctgaacagagaggatctgctg | |
| | agaaagcaaagaactttcgataacggatccatcccacaccaaatccacct | |
| | gggagagctgcacgctatcctgaggagacaagaggatttctacccattcc | |
| | tgaaggataacagagagaagatcgagaagatcctgactttcagaatccca | |
| | tactacgtgggaccactggctagaggaaactccagattcgcttggatgac | |
| | tagaaagtccgaggagactatcactccatggaacttcgaggaggtaagtt | |
| | tctgcttctacctttgatatatatataataattatcattaattagtagta | |
| | atataatatttcaaatatttttttcaaaataaaagaatgtagtatatagc | |
| | aattgcttttctgtagtttataagtgtgtatattttaatttataactttt | |
| | ctaatatatgaccaaaatttgttgatgtgcaggtggtggataagggagct | |
| | tccgctcaatccttcatcgagagaatgactaacttcgataagaacctgcc | |
| | aaacgagaaggtgctgccaaagcactccctgctgtacgagtacttcactg | |
| | tgtacaacgagctgactaaggtgaagtacgtgactgagggaatgagaaag | |
| | ccagctttcctgtccggagagcaaaagaaggctatcgtggatctgctgtt | |
| | caagactaacagaaaggtgactgtgaagcaactgaaggaggattacttca | |
| | agaagatcgagtgcttcgattccgtggagatctccggagtggaggataga | |
| | ttcaacgcttccctgggaacttaccacgatctgctgaagatcatcaagga | |
| | taaggatttcctggataacgaggagaacgaggatatcctggaggatatcg | |
| | tgctgactctgactctgttcgaggatagagagatgatcgaggagagactt | |
| | aagacttacgctcacctgttcgatgataaggtgatgaagcaactgaagag | |
| | aagaagatacactggatggggtagactgtccagaaagctgatcaacggaa | |
| | tcagagataagcaatccggaaagactatcctggatttcctgaagtccgat | |
| | ggattcgctaacagaaacttcatgcaactgatccacgatgattccctgac | |
| | tttcaaggaggatatccaaaaggctcaagtgtccggacaaggagattccc | |
| | tgcacgagcacatcgctaacctggctggatccccagctatcaagaaggga | |
| | atcctgcaaactgtgaaggtggtggatgagctggtgaaggtgatgggtag | |
| | acacaagccagagaacatcgtgatcgagatggctagagagaaccaaacta | |
| | ctcaaaagggacaaaagaactccagagagagaatgaagagaatcgaggag | |
| | ggaatcaaggagctgggatcccaaatcctgaaggagcacccagtggagaa | |
| | cactcaactgcaaaacgagaagctgtacctgtactacctgcaaaacggaa | |
| | gagatatgtacgtggatcaagagctggatatcaacagactgtccgattac | |
| | gatgtggatgcaatcgtgccacaatccttcctgaaggatgattccatcga | |
| | taacaaggtgctgactagatccgataagaacagaggaaagtccgataacg | |
| | tgccatccgaggaggtggtgaagaagatgaagaactactggagacaactg | |
| | ctgaacgctaagctgatcactcaaagaaagttcgataacctgactaaggc | |
| | tgagagaggaggactgtccgagctggataaggctggattcatcaagagac | |
| | aactggtggagactagacaaatcactaagcacgtggctcaaatcctggat | |
| | tccagaatgaacactaagtacgatgagaacgataagctgatcagagaggt | |
| | gaaggtgatcactctgaagtccaagctggtgtccgatttcagaaaggatt | |
| | tccaattctacaaggtgagagagatcaacaactaccaccacgctcacgat | |
| | gcttacctgaacgctgtggtgggaactgctctgatcaagaagtacccaaa | |
| | gctggagtccgagttcgtgtacggagattacaaggtgtacgatgtgagaa | |
| | agatgatcgctaagtccgagcaagagatcggaaaggctactgctaagtac | |
| | ttcttctactccaacatcatgaacttcttcaagactgagatcactctggc | |
| | taacggagagatcagaaaaagaccactgatcgagactaacggagagactg | |
| | gagagatcgtgtgggataagggaagagatttcgctactgtgagaaaggtg | |
| | ctgtccatgccacaagtgaacatcgtgaaaaagactgaggtgcaaactgg | |
| | aggattctccaaggagtccatcctgccaaagagaaactccgataagctga | |
| | tcgctagaaagaaggattgggatccaaagaagtacggaggattcgattcc | |
| | ccaactgtggcttactccgtgctggtggtggctaaggtggagaagggaaa | |
| | gtccaagaagctgaagtccgtgaaggagctgctgggaatcactatcatgg | |
| | agagatcctccttcgagaagaacccaatcgatttcctggaggctaaggga | |
| | tacaaggaggtgaagaaggatctgatcatcaagctgccaaagtactccct | |
| | gttcgagctggagaacggaagaaagagaatgctggcttccgctggagagc | |
| | tgcaaaagggaaacgagctggctctgccatccaagtacgtgaacttcctg | |
| | tacctggcttcccactacgagaagctgaagggatccccagaggataacga | |
| | gcaaaagcaactgttcgtggagcaacacaagcactacctggatgagatca | |
| | tcgagcaaatctccgagttctccaagagagtgatcctggctgatgctaac | |
| | ctggataaggtgctgtccgcttacaacaagcacagagataagccaatcag | |
| | agagcaagctgagaacatcatccacctgttcactctgactaacctgggag | |
| | ctccagctgctttcaagtacttcgatactactatcgatagaaagagatac | |
| | acttccactaaggaggtgctggatgctactctgatccaccaatccatcac | |
| | tggactgtacgagactagaatcgatctgtcccaactgggaggagatagtt | |
| | cgaagcgcccagccgctaccaaaaaagctggacaggctaaaaaaaagaag | |
| | ggtgacggcagcgggtccggctccgggtccggttccgtcgatctcgattt | |
| | ggatctggaacttcgcctggggttcgcactcgatctcgaccttgaactgc | |
| | gtcttggctttgctctcgatcttgatctggagttgaggcttggattcgct | |
| | tag | |
| 69 | gtgccgaattcggatccagcgatctggattttagtactggattttggttt | RC4270 |
| | taggaattagaaattttattgatagaagtattttacaaatacaaatacat | M. Frank |
| | actaagggtttcttatatgctcaacacatgagcgaaaccctataggaacc | AGL1- |
| | ctaattcccttatctgggaactactcacacattattatggagaaactcga | P5U |
| | gcttgtcgatcgactctagctagagaagctcactttccaacaaccttagt | dCas9-TV |
| | aatttctcccttaacataatgaacaaattcttcaagttgatcagctctag | CONTROL |
| | aagcaagtctatcttcttcttgtccaagataagcttgtctagcttcaaga | Construct |
| | ataactggttgatattgagctggaagtctttccatagcccaatcagcagc | (RCGG30 |
| | aacatcctttggagcaatctttccagtaacagcagaataccaaattctag | 03) |
| | aaagagtaagaacaacatttctttcatctccagcccaatctggtggagaa | Plasmid |
| | ttccaaagagtaagagtttcattaagagcttcaaaaagatcttgttctgg | comprising |
| | aactggatcaaaaagttcttcagcagctggtccaacaagagcaacagaat | expression |
| | gttctctagccttagtaagaagaatagcaagatcaatatcaatagtagct | construct |
| | ggttcaaaaattccagcaagaatatcatttctttgccattctccaaattg | of SEQ ID |
| | aagttctctcttagctggatatctccatggaataatatcatcatgaacaa | NO: 63 |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagatatttgaa | |
| | tatacttaatgttgatgttaaatattatttcatccatcttaaaataagtg | |
| | tcattctatttattttatatagacaaaaaaaagttaataaattaataaaa | |
| | aaacagtaattttataaaactaaccttagaattaatatttcattaaaaaa | |
| | ctaaaattacacttattagaaatattagaaaaaaaatatcactactaaaa | |
| | atattattttttatgacgaacattctaaaatggttatatgaaatcatttt | |
| | agaatgtcatgcgatgacaattgtaaatacaagattgtttaacaaagacg | |
| | attttacaaaaattgtcttaaaaattattgttttatgacggtttgatgaa | |
| | aaccgtcttaaaatgattgtcattctaaaacgattttcgtaaaatcctct | |
| | taaaagttagtgcttttaatttttatgtctccctctcctctctcatttct | |
| | ctccctctcactctctactacttcttccgttccaaaataattgttgtatt | |
| | aggttattttacataggtgaagtaataaatagatgaaatataatgataat | |
| | tttagaaaattaatcttatatcatcatttaatacatttatatatagttca | |
| | tcattaataaataaaaaaataattaatgttacatttaaaaactaaaatga | |
| | taaaagtaattaatatacattaagaaactaaaatgacatatttttcttat | |
| | acgacaattataacacgatgagggagtaatgaatattagttaaaattagt | |
| | ttttgatcacctaatttataattttgattcaatttagtcctttagttatt | |
| | ttgggttcaatttaatcctctaatttttaaaattattttttttactttcc | |
| | attccatcacaagttccaaatgaatttgaatttaatcaacttatggtagt | |
| | atatatagaactttgaaagattattatctattgtaaaaaaattcaatctt | |
| | aagattttttttgcataacatgattcctaaactactcatcaggtgtctta | |
| | aagatcagttactagtgtaatggagctagctacttggctcacatttcagg | |
| | tattttacgtaaaagtccttacatgcactgtcgccctaacactttgttta | |
| | tgctaaccttgtcatcaaaatcaaaatgcagaaaagggaaaagaaagaaa | |
| | aaattcaaactggctttcccatccccataaagacttgctaaatataagga | |
| | gaagaagtgaagattatgatcagacatcacgtgggaacccttcttagtta | |
| | aaattgtaataattcaagagagagggtcaagagtcactggaaactctcag | |
| | gtttcaattttcaactgtacaaaataaaaattaaagagatgcaggggtca | |
| | gttaaaggaaataaatcttgtagatataaatatctatatatagattatgg | |
| | ttggataactttttatagaagaagaaaagaaaaacttgtcttataaattt | |
| | aaatcatcttacacatgcatgcacttgaattttatagaaactcttataag | |
| | ctaaatatataaattaattttaatttatgaaaatatatagtttgcttcat | |
| | tttactatttatttacttttgttataagtgtaaacaaggccatattcgaa | |
| | gtaaaaggccaaaaactcaaaatccaagaagcaaaatggtttctttattt | |
| | tttttatgctttaaaaatttgaagcttcggaattttgcgtgcaaactttt | |
| | atgtgcatgtccctctcattgaaattgaatcaaaccccacattaacaaat | |
| | taaagtcactttcgaaaagaccctttgattatctccctctcctttttccc | |
| | atacatgaagaaatggatggtttatggagcatgaaaggtgaacctcaagt | |
| | ctctctctctctcttctctctcatgcttaaattctccaacacgagagtaa | |
| | attttagagtgagtggtaattgtggagggtctatttttctgtcataaata | |
| | cctgtctgctaggccaacccacttccatattttgcatcctttgtacctca | |
| | gttccaggttaacaccaaacaacacatatagtgcagattcataaggtata | |
| | gactttgttaatttgcaccctctcttaccctaaatcttgtgtctattgta | |
| | ttttatctctttccccatggattacaaagatgacgatgataaagactaca | |
| | aggacgacgacgataaaatggcccctaaaaagaagcgcaaggtgggtatt | |
| | catggggtgcctgcagctggaatggataagaagtactccatcggactggc | |
| | tatcggaactaactccgtgggatgggctgtgatcactgatgagtacaagg | |
| | tgccatccaagaagttcaaggtgctgggaaacactgatagacactccatc | |
| | aagaagaacctgatcggagctctgctgttcgattccggagagactgctga | |
| | ggctactagactgaagagaactgctagaagaagatacactagaagaaaga | |
| | acagaatctgctacctgcaagagatcttctccaacgagatggctaaggtg | |
| | gatgattccttcttccacagactggaggagtccttcctggtggaggagga | |
| | taagaagcacgagagacacccaatcttcggaaacatcgtggatgaggtgg | |
| | cttaccacgagaagtacccaactatctaccacctgagaaagaagctggtg | |
| | gattccactgataaggctgatctgagactgatctacctggctctggctca | |
| | catgatcaagttcagaggacacttcctgatcgagggagatctgaacccag | |
| | ataactccgatgtggataagctgttcatccaactggtgcaaacttacaac | |
| | caactgttcgaggagaacccaatcaacgcttccggagtggatgctaaggc | |
| | tatcctgtccgctagactgtccaagtccaggagactggagaacctgatcg | |
| | ctcaactgccaggagagaagaagaacggactgttcggaaacctgatcgct | |
| | ctgtccctgggactgactccaaacttcaagtccaacttcgatctggctga | |
| | ggatgctaagctgcaactgtccaaggatacttacgatgatgatctggata | |
| | acctgctggctcaaatcggagatcaatacgctgatctgttcctggctgct | |
| | aagaacctgtccgatgctatcctgctgtccgatatcctgagagtgaacac | |
| | tgagatcactaaggctccactgtccgcttccatgatcaagagatacgatg | |
| | agcaccaccaagatctgactctgctgaaggctctggtgagacaacaactg | |
| | ccagagaagtacaaggagatcttcttcgatcaatccaagaacggatacgc | |
| | tggatacatcgatggaggagcttcccaagaggagttctacaagttcatca | |
| | agccaatcctggagaagatggatggaactgaggagctgctggtgaagctg | |
| | aacagagaggatctgctgagaaagcaaagaactttcgataacggatccat | |
| | cccacaccaaatccacctgggagagctgcacgctatcctgaggagacaag | |
| | aggatttctacccattcctgaaggataacagagagaagatcgagaagatc | |
| | ctgactttcagaatcccatactacgtgggaccactggctagaggaaactc | |
| | cagattcgcttggatgactagaaagtccgaggagactatcactccatgga | |
| | acttcgaggaggtaagtttctgcttctacctttgatatatatataataat | |
| | tatcattaattagtagtaatataatatttcaaatatttttttcaaaataa | |
| | aagaatgtagtatatagcaattgcttttctgtagtttataagtgtgtata | |
| | ttttaatttataacttttctaatatatgaccaaaatttgttgatgtgcag | |
| | gtggtggataagggagcttccgctcaatccttcatcgagagaatgactaa | |
| | cttcgataagaacctgccaaacgagaaggtgctgccaaagcactccctgc | |
| | tgtacgagtacttcactgtgtacaacgagctgactaaggtgaagtacgtg | |
| | actgagggaatgagaaagccagctttcctgtccggagagcaaaagaaggc | |
| | tatcgtggatctgctgttcaagactaacagaaaggtgactgtgaagcaac | |
| | tgaaggaggattacttcaagaagatcgagtgcttcgattccgtggagatc | |
| | tccggagtggaggatagattcaacgcttccctgggaacttaccacgatct | |
| | gctgaagatcatcaaggataaggatttcctggataacgaggagaacgagg | |
| | atatcctggaggatatcgtgctgactctgactctgttcgaggatagagag | |
| | atgatcgaggagagacttaagacttacgctcacctgttcgatgataaggt | |
| | gatgaagcaactgaagagaagaagatacactggatggggtagactgtcca | |
| | gaaagctgatcaacggaatcagagataagcaatccggaaagactatcctg | |
| | gatttcctgaagtccgatggattcgctaacagaaacttcatgcaactgat | |
| | ccacgatgattccctgactttcaaggaggatatccaaaaggctcaagtgt | |
| | ccggacaaggagattccctgcacgagcacatcgctaacctggctggatcc | |
| | ccagctatcaagaagggaatcctgcaaactgtgaaggtggtggatgagct | |
| | ggtgaaggtgatgggtagacacaagccagagaacatcgtgatcgagatgg | |
| | ctagagagaaccaaactactcaaaagggacaaaagaactccagagagaga | |
| | atgaagagaatcgaggagggaatcaaggagctgggatcccaaatcctgaa | |
| | ggagcacccagtggagaacactcaactgcaaaacgagaagctgtacctgt | |
| | actacctgcaaaacggaagagatatgtacgtggatcaagagctggatatc | |
| | aacagactgtccgattacgatgtggatgcaatcgtgccacaatccttcct | |
| | gaaggatgattccatcgataacaaggtgctgactagatccgataagaaca | |
| | gaggaaagtccgataacgtgccatccgaggaggtggtgaagaagatgaag | |
| | aactactggagacaactgctgaacgctaagctgatcactcaaagaaagtt | |
| | cgataacctgactaaggctgagagaggaggactgtccgagctggataagg | |
| | ctggattcatcaagagacaactggtggagactagacaaatcactaagcac | |
| | gtggctcaaatcctggattccagaatgaacactaagtacgatgagaacga | |
| | taagctgatcagagaggtgaaggtgatcactctgaagtccaagctggtgt | |
| | ccgatttcagaaaggatttccaattctacaaggtgagagagatcaacaac | |
| | taccaccacgctcacgatgcttacctgaacgctgtggtgggaactgctct | |
| | gatcaagaagtacccaaagctggagtccgagttcgtgtacggagattaca | |
| | aggtgtacgatgtgagaaagatgatcgctaagtccgagcaagagatcgga | |
| | aaggctactgctaagtacttcttctactccaacatcatgaacttcttcaa | |
| | gactgagatcactctggctaacggagagatcagaaaaagaccactgatcg | |
| | agactaacggagagactggagagatcgtgtgggataagggaagagatttc | |
| | gctactgtgagaaaggtgctgtccatgccacaagtgaacatcgtgaaaaa | |
| | gactgaggtgcaaactggaggattctccaaggagtccatcctgccaaaga | |
| | gaaactccgataagctgatcgctagaaagaaggattgggatccaaagaag | |
| | tacggaggattcgattccccaactgtggcttactccgtgctggtggtggc | |
| | taaggtggagaagggaaagtccaagaagctgaagtccgtgaaggagctgc | |
| | tgggaatcactatcatggagagatcctccttcgagaagaacccaatcgat | |
| | ttcctggaggctaagggatacaaggaggtgaagaaggatctgatcatcaa | |
| | gctgccaaagtactccctgttcgagctggagaacggaagaaagagaatgc | |
| | tggcttccgctggagagctgcaaaagggaaacgagctggctctgccatcc | |
| | aagtacgtgaacttcctgtacctggcttcccactacgagaagctgaaggg | |
| | atccccagaggataacgagcaaaagcaactgttcgtggagcaacacaagc | |
| | actacctggatgagatcatcgagcaaatctccgagttctccaagagagtg | |
| | atcctggctgatgctaacctggataaggtgctgtccgcttacaacaagca | |
| | cagagataagccaatcagagagcaagctgagaacatcatccacctgttca | |
| | ctctgactaacctgggagctccagctgctttcaagtacttcgatactact | |
| | atcgatagaaagagatacacttccactaaggaggtgctggatgctactct | |
| | gatccaccaatccatcactggactgtacgagactagaatcgatctgtccc | |
| | aactgggaggagatagttcgaagaggcctgccgccacaaagaaagccggc | |
| | caggcaaagaaaaaaaaacccaggggagggagtggtggactcctcgaccc | |
| | tggcactccaatggacgccgatctcgtagcatcatctaccgtcgtgtggg | |
| | agcaggacgccgacccttttgctggcacagctgatgatttcccagctttt | |
| | aatgaggaagaattggcttggctgatggagcttctcccacaaggtggtag | |
| | cggcgggctcctcgatcccggtactcccatggacgcagatcttgttgcta | |
| | gctccactgttgtatgggagcaggacgccgatcccttcgctggaaccgcc | |
| | gatgatttcccagcattcaacgaggaagaacttgcatggctcatggagct | |
| | cttgccccaagctcgcggtggatctgggggtttgttggatcccggaaccc | |
| | caatggacgcagacctcgtggcttctagtaccgtagtatgggaacaggac | |
| | gctgaccctttcgccggcaccgcagatgacttcccagcattcaacgagga | |
| | ggaactggcctggctgatggaacttctgcctcagggtggatccggaggcc | |
| | tcttggaccccggtacccctatggacgctgacctggtcgcatcttctacc | |
| | gtggtttgggagcaagatgccgacccattcgcaggaacagctgatgactt | |
| | ccccgcattcaatgaagaagagctcgcatggctgatggaattgcttccac | |
| | aagccagaggcggctctggagggcttcttgatcccggaacacccatggat | |
| | gccgacctcgtggcctctagcaccgtggtatgggaacaagacgcagatcc | |
| | attcgcagggaccgcagacgatttccccgcttttaacgaggaggagcttg | |
| | cttggctcatggagctgctgcctcagggtggatccgggggacttctcgac | |
| | ccaggcacacccatggatgccgatctcgtcgccagttctaccgttgtctg | |
| | ggagcaagacgcagaccctttcgccggaactgcagacgacttccctgcct | |
| | ttaatgaagaagaactggcttggctcatggaactgctcccccaggctaga | |
| | gggggtagcggcgggggcggcagcggaggtgatgcacttgacgacttcga | |
| | tcttgatatgcttggcagcgacgcacttgatgacttcgatttggatatgc | |
| | tggggtccgatgctcttgacgattttgatttggacatgttgggttcagac | |
| | gcactcgatgatttcgatttggacatgctggcacgtggaagcgatgccct | |
| | tgatgactttgacctggatatgctcgggtccgatgctttggacgacttcg | |
| | acctcgacatgttgggaagtgatgctcttgatgatttcgatcttgatatg | |
| | cttgggtccgacgctctcgatgattttgacctcgatatgctctgagcttg | |
| | tcaagcagatcgttcaaacatttggcaataaagtttcttaagattgaatc | |
| | ctgttgccggtcttgcgatgattatcatataatttctgttgaattacgtt | |
| | aagcatgtaataattaacatgtaatgcatgacgttatttatgagatgggt | |
| | ttttatgattagagtcccgcaattatacatttaatacgcgatagaaaaca | |
| | aaatatagcgcgcaaactaggataaattatcgcgcgcggtgtcatctatg | |
| | ttactagatcgacgctttacgaattcccatggggagatatttgaatatac | |
| | ttaatgttgatgttaaatattatttcatccatcttaaaataagtgtcatt | |
| | ctatttattttatatagacaaaaaaaagttaataaattaataaaaaaaca | |
| | gtaattttataaaactaaccttagaattaatatttcattaaaaaactaaa | |
| | attacacttattagaaatattagaaaaaaaatatcactactaaaaatatt | |
| | attttttatgacgaacattctaaaatggttatatgaaatcattttagaat | |
| | gtcatgcgatgacaattgtaaatacaagattgtttaacaaagacgatttt | |
| | acaaaaattgtcttaaaaattattgttttatgacggtttgatgaaaaccg | |
| | tcttaaaatgattgtcattctaaaacgattttcgtaaaatcctcttaaaa | |
| | gttagtgcttttaatttttatgtctccctctcctctctcatttctctccc | |
| | tctcactctctactacttcttccgttccaaaataattgttgtattaggtt | |
| | attttacataggtgaagtaataaatagatgaaatataatgataattttag | |
| | aaaattaatcttatatcatcatttaatacatttatatatagttcatcatt | |
| | aataaataaaaaaataattaatgttacatttaaaaactaaaatgataaaa | |
| | gtaattaatatacattaagaaactaaaatgacatatttttcttatacgac | |
| | aattataacacgatgagggagtaatgaatattagttaaaattagtttttg | |
| | atcacctaatttataattttgattcaatttagtcctttagttattttggg | |
| | ttcaatttaatcctctaatttttaaaattattttttttactttccattcc | |
| | atcacaagttccaaatgaatttgaatttaatcaacttatggtagtatata | |
| | tagaactttgaaagattattatctattgtaaaaaaattcaatcttaagat | |
| | tttttttgcataacatgattcctaaactactcatcaggtgtcttaaagat | |
| | cagttactagtgtaatggagctagctacttggctcacatttcaggtattt | |
| | tacgtaaaagtccttacatgcactgtcgccctaacactttgtttatgcta | |
| | accttgtcatcaaaatcaaaatgcagaaaagggaaaagaaagaaaaaatt | |
| | caaactggctttcccatccccataaagacttgctaaatataaggagaaga | |
| | agtgaagattatgatcagacatcacgtgggaacccttcttagttaaaatt | |
| | gtaataattcaagagagagggtcaagagtcactggaaactctcaggtttc | |
| | aattttcaactgtacaaaataaaaattaaagagatgcaggggtcagttaa | |
| | aggaaataaatcttgtagatataaatatctatatatagattatggttgga | |
| | taactttttatagaagaagaaaagaaaaacttgtcttataaatttaaatc | |
| | atcttacacatgcatgcacttgaattttatagaaactcttataagctaaa | |
| | tatataaattaattttaatttatgaaaatatatagtttgcttcattttac | |
| | tatttatttacttttgttataagtgtaaacaaggccatattcgaagtaaa | |
| | aggccaaaaactcaaaatccaagaagcaaaatggtttctttatttttttt | |
| | atgctttaaaaatttgaagcttcggaattttgcgtgcaaacttttatgtg | |
| | catgtccctctcattgaaattgaatcaaaccccacattaacaaattaaag | |
| | tcactttcgaaaagaccctttgattatctccctctcctttttcccataca | |
| | tgaagaaatggatggtttatggagcatgaaaggtgaacctcaagtctctc | |
| | tctctctcttctctctcatgcttaaattctccaacacgagagtaaatttt | |
| | agagtgagtggtaattgtggagggtctatttttctgtcataaatacctgt | |
| | ctgctaggccaacccacttccatattttgcatcctttgtacctcagttcc | |
| | aggttaacaccaaacaacacatatagtgcagattcataaggtatagactt | |
| | tgttaatttgcaccctctcttaccctaaatcttgtgtctattgtatttta | |
| | tctctttccaatgggtcagtcccttatgttacgtcctgtagaaaccccaa | |
| | cccgtgaaatcaaaaaactcgacggcctgtgggcattcagtctggatcgc | |
| | gaaaactgtggaattgatcagcgttggtgggaaagcgcgttacaagaaag | |
| | ccgggcaattgctgtgccaggcagttttaacgatcagttcgccgatgcag | |
| | atattcgtaattatgcgggcaacgtctggtatcagcgcgaagtctttata | |
| | ccgaaaggtaagtcttactctctcttttttggtctgtatttttaattttt | |
| | tgaagtatactatttgtactgacgctaataatcttttttcaggttgggca | |
| | ggccagcgtatcgtgctgcgtttcgatgcggtcactcattacggcaaagt | |
| | gtgggtcaataatcaggaagtgatggagcatcagggcggctatacgccat | |
| | ttgaagccgatgtcacgccgtatgttattgccgggaaaagtgtacgtatc | |
| | accgtttgtgtgaacaacgaactgaactggcagactatcccgccgggaat | |
| | ggtgattaccgacgaaaacggcaagaaaaagcagtcttacttccatgatt | |
| | tctttaactatgccggaatccatcgcagcgtaatgctctacaccacgccg | |
| | aacacctgggtggacgatatcaccgtggtgacgcatgtcgcgcaagactg | |
| | taaccacgcgtctgttgactggcaggtacttcatgcttcaacgtgtaact | |
| | taagagatactgtgtgaaattttatatttccatacatttgcttgaccttt | |
| | gctttttgtcaatttttttccccttacaggtggtggccaatggtgatgtc | |
| | agcgttgaactgcgtgatgcggatcaacaggtggttgcaactggacaagg | |
| | cactagcgggactttgcaagtggtgaatccgcacctctggcaaccgggtg | |
| | aaggttatctctatgaactgtgcgtcacagccaaaagccagacagagtgt | |
| | gatatctacccgcttcgcgtcggcatccggtcagtggcagtgaagggcga | |
| | acagttcctgattaaccacaaaccgttctactttactggctttggtcgtc | |
| | atgaagatgcggacttgcgtggcaaaggattcgataacgtgctgatggtg | |
| | cacgaccacgcattaatggactggattggggccaactcctaccgtacctc | |
| | gcattacccttacgctgaagagatgctcgactgggcagatgaacatggca | |
| | tcgtggtgattgatgaaactgctgctgtcggctttaacctctctttaggc | |
| | attggtttcgaagcgggcaacaagccgaaagaactgtacagcgaagaggc | |
| | agtcaacggggaaactcagcaagcgcacttacaggcgattaaagagctga | |
| | tagcgcgtgacaaaaaccacccaagcgtggtgatgtggagtattgccaac | |
| | gaaccggatacccgtccgcaaggtgcacgggaatatttcgcgccactggc | |
| | ggaagcaacgcgtaaactcgacccgacgcgtccgatcacctgcgtcaatg | |
| | taatgttctgcgacgctcacaccgataccatcagcgatctctttgatgtg | |
| | ctgtgcctgaaccgttattacggatggtatgtccaaagcggcgatttgga | |
| | aacggcagagaaggtactggaaaaagaacttctggcctggcaggagaaac | |
| | tgcatcagccgattatcatcaccgaatacggcgtggatacgttagccggg | |
| | ctgcactcaatgtacaccgacatgtggagtgaagagtatcagtgtgcatg | |
| | gctggatatgtatcaccgcgtctttgatcgcgtcagcgccgtcgtcggtg | |
| | aacaggtatggaatttcgccgattttgcgacctcgcaaggcatattgcgc | |
| | gttggcggtaacaagaaagggatcttcactcgcgaccgcaaaccgaagtc | |
| | ggcggcttttctgctgcaaaaacgctggactggcatgaacttcggtgaaa | |
| | aaccgcagcagggaggcaaacaaggttcggtgtccaagggcgaagaggtg | |
| | attaaagagttcatgaggttcaaagtgagaatggagggcagtatgaacgg | |
| | ccatgagttcgagatcgaaggtgaaggcgaaggtagaccatacgagggca | |
| | cccagacggcgaaactgaaagtgacgaagggcggcccactgcccttcgcc | |
| | tgggacattctgagtccacaatttatgtacggttccaaggcatacgttaa | |
| | gcaccccgccgacatccctgattacaaaaagctgtcgttcccggaggggt | |
| | ttaaatgggaaagagtgatgaattttgaggacggagggctcgtcactgtg | |
| | actcaagactcctcacttcaagacggcaccctaatttacaaggtgaagat | |
| | gaggggaacaaatttcccccctgatggccctgttatgcagaaaaaaacaa | |
| | tgggatgggaggcatccaccgagagactttacccgcgcgacggagtgctc | |
| | aaaggcgaaatccatcaggcactgaaactaaaggacggcggccactacct | |
| | ggttgagttcaaaactatatatatggccaagaaacccgtccagttgccgg | |
| | gatattactatgtggacacaaaactggatataacgtcccacaacgaagat | |
| | tacaccatagtggaacagtatgagcgcagtgagggtcgtcaccatctatt | |
| | cctcggacatggcacgggttccacgggctcgggttccagcggcacggcaa | |
| | gctcggaggacaacaacatggcggtgatcaaagagttcatgcggtttaaa | |
| | gtccgtatggagggcagcatgaatgggcacgaatttgaaattgagggaga | |
| | aggcgagggccgaccatatgaggggacacaaaccgcgaaactaaaagtca | |
| | ccaaaggtgggccgcttccttttgcctgggacattctctctcctcagttc | |
| | atgtatggctccaaggcctacgttaaacatccggctgatatccccgatta | |
| | taagaaattgtctttcccggaggggtttaaatgggagcgggttatgaatt | |
| | tcgaggatggtggactggttactgttacacaagattcgagtctgcaggat | |
| | ggcacattgatatacaaagtcaaaatgaggggtacgaattttcccccgga | |
| | cggcccggtgatgcagaaaaaaactatgggctgggaggccagcacggaga | |
| | ggctgtacccgagggacggggttttgaaaggggagatacaccaggcgctt | |
| | aaactgaaggacggggggcactatctggtggaatttaagacgatatatat | |
| | ggcgaaaaagcccgtgcagctacctggctactactacgtcgataccaaac | |
| | tcgatattacctctcataacgaggactacactatcgtggagcaatatgaa | |
| | agatcagaaggacgtcaccacctttttctatacggaatggacgagcttta | |
| | caaagcttgtgtgtcttgtcttatctggttcgtggtggtgagtttgttac | |
| | aaaaaaatctattttccctagttgagatgggaattgaactatctgttgtt | |
| | atgtggattttattttcttttttctctttagaaccttatggttgtgtcaa | |
| | gaagtcttgtgtactttagttttatatctctgttttatctcttctatttt | |
| | ctttaggatgcttgtgatgatgctgtttttttttgtccctaagcaaaaaa | |
| | atatcatattatatttggtccttggttcatttttttggtttttttttatc | |
| | ttcacatataaatattgtttgaatatcttcaatcttttatttgtatgaga | |
| | caattatttaagtatcgggtgacaatgcagctattatgtattgtcgattg | |
| | ttcgctcagagaggatgcacatgtgaccgagggacacgaagtgatccgtt | |
| | taaactatcagtgtttgacaggatatattgggggtaaacctaagagaaaa | |
| | gagcgtttattagaataatcggatatttaaaagggcgtgaaaaggtttat | |
| | ccgttcgtccatttgtatgtgccagccgcctttgcgacgctcaccgggct | |
| | ggttgccctcgccgctgggctggcggccgtctatggccctgcaaacgcgc | |
| | cagaaacgccgtcgaagccgtgtgcgagacaccgcggccgccggcgttgt | |
| | ggatacctcgcggaaaacttggccctcactgacagatgaggggcggacgt | |
| | tgacacttgaggggccgactcacccggcgcggcgttgacagatgaggggc | |
| | aggctcgatttcggccggcgacgtggagctggccagcctcgcaaatcggc | |
| | gaaaacgcctgattttacgcgagtttcccacagatgatgtggacaagcct | |
| | ggggataagtgccctgcggtattgacacttgaggggcgcgactactgaca | |
| | gatgaggggcgcgatccttgacacttgaggggcagagtgctgacagatga | |
| | ggggcgcacctattgacatttgaggggctgtccacaggcagaaaatccag | |
| | catttgcaagggtttccgcccgtttttcggccaccgctaacctgtctttt | |
| | aacctgcttttaaaccaatatttataaaccttgtttttaaccagggctgc | |
| | gccctgtgcgcgtgaccgcgcacgccgaaggggggtgcccccccttctcg | |
| | aaccctcccggcccgctaacgcgggcctcccatccccccaggggctgcgc | |
| | ccctcggccgcgaacggcctcaccccaaaaatggcagcgctggccaattc | |
| | ccgagtgcgcggaacccctatttgtttatttttctaaatacattcaaata | |
| | tgtatccgctcatgagacaataaccctgataaatgcttcaataatattga | |
| | aaaaggaagagtatggctaaaatgagaatatcaccggaattgaaaaaact | |
| | gatcgaaaaataccgctgcgtaaaagatacggaaggaatgtctcctgcta | |
| | aggtatataagctggtgggagaaaatgaaaacctatatttaaaaatgacg | |
| | gacagccggtataaagggaccacctatgatgtggaacgggaaaaggacat | |
| | gatgctatggctggaaggaaagctgcctgttccaaaggtcctgcactttg | |
| | aacggcatgatggctggagcaatctgctcatgagtgaggccgatggcgtc | |
| | ctttgctcggaagagtatgaagatgaacaaagccctgaaaagattatcga | |
| | gctgtatgcggagtgcatcaggctctttcactccatcgacatatcggatt | |
| | gtccctatacgaatagcttagacagccgcttagccgaattggattactta | |
| | ctgaataacgatctggccgatgtggattgcgaaaactgggaagaggacac | |
| | tccatttaaagatccgcgcgagctgtatgattttttaaagacggaaaagc | |
| | ccgaagaggaacttgtcttttcccacggcgacctgggagacagcaacatc | |
| | tttgtgaaagatggcaaagtaagtggctttattgatcttgggagaagcgg | |
| | cagggcggacaagtggtatgacattgccttctgcgtccggtcgctcaggg | |
| | aggatatcggggaagaacagtatgtcgagctattttttgacttactgggg | |
| | atcaagcctgattgggagaaaataaaatattatattttactggatgaatt | |
| | gttttagctgtcagaccaagtttactcatatatactttagattgatttaa | |
| | aacttcatttttaatttaaaaggatctaggtgaagatcctttttgataat | |
| | ctcatgaccaaaatcccttaacgtgagttttcgttccactgagcgtcaga | |
| | ccccgtagaaaagatcaaaggatcttcttgagatcctttttttctgcgcg | |
| | taatctgctgcttgcaaacaaaaaaaccaccgctaccagcggtggtttgt | |
| | ttgccggatcaagagctaccaactctttttccgaaggtaactggcttcag | |
| | cagagcgcagataccaaatactgtccttctagtgtagccgtagttaggcc | |
| | accacttcaagaactctgtagcaccgcctacatacctcgctctgctaatc | |
| | ctgttaccagtggctgctgccagtggcgataagtcgtgtcttaccgggtt | |
| | ggactcaagacgatagttaccggataaggcgcagcggtcgggctgaacgg | |
| | ggggttcgtgcacacagcccagcttggagcgaacgacctacaccgaactg | |
| | agatacctacagcgtgagctatgagaaagcgccacgcttcccgaagggag | |
| | aaaggcggacaggtatccggtaagcggcagggtcggaacaggagagcgca | |
| | cgagggagcttccagggggaaacgcctggtatctttatagtcctgtcggg | |
| | tttcgccacctctgacttgagcgtcgatttttgtgatgctcgtcaggggg | |
| | gcggagcctatggaaaaacgccagcaacgcggcctttttacggttcctgg | |
| | cagatcctagatgtggcgcaacgatgccggcgacaagcaggagcgcaccg | |
| | acttcttccgcatcaagtgttttggctctcaggccgaggcccacggcaag | |
| | tatttgggcaaggggtcgctggtattcgtgcagggcaagattcggaatac | |
| | caagtacgagaaggacggccagacggtctacgggaccgacttcattgccg | |
| | ataaggtggattatctggacaccaaggcaccaggcgggtcaaatcaggaa | |
| | taagggcacattgccccggcgtgagtcggggcaatcccgcaaggagggtg | |
| | aatgaatcggacgtttgaccggaaggcatacaggcaagaactgatcgacg | |
| | cggggttttccgccgaggatgccgaaaccatcgcaagccgcaccgtcatg | |
| | cgtgcgccccgcgaaaccttccagtccgtcggctcgatggtccagcaagc | |
| | tacggccaagatcgagcgcgacagcgtgcaactggctccccctgccctgc | |
| | ccgcgccatcggccgccgtggagcgttcgcgtcgtcttgaacaggaggcg | |
| | gcaggtttggcgaagtcgatgaccatcgacacgcgaggaactatgacgac | |
| | caagaagcgaaaaaccgccggcgaggacctggcaaaacaggtcagcgagg | |
| | ccaagcaggccgcgttgctgaaacacacgaagcagcagatcaaggaaatg | |
| | cagctttccttgttcgatattgcgccgtggccggacacgatgcgagcgat | |
| | gccaaacgacacggcccgctctgccctgttcaccacgcgcaacaagaaaa | |
| | tcccgcgcgaggcgctgcaaaacaaggtcattttccacgtcaacaaggac | |
| | gtgaagatcacctacaccggcgtcgagctgcgggccgacgatgacgaact | |
| | ggtgtggcagcaggtgttggagtacgcgaagcgcacccctatcggcgagc | |
| | cgatcaccttcacgttctacgagctttgccaggacctgggctggtcgatc | |
| | aatggccggtattacacgaaggccgaggaatgcctgtcgcgcctacaggc | |
| | gacggcgatgggcttcacgtccgaccgcgttgggcacctggaatcggtgt | |
| | cgctgctgcaccgcttccgcgtcctggaccgtggcaagaaaacgtcccgt | |
| | tgccaggtcctgatcgacgaggaaatcgtcgtgctgtttgctggcgacca | |
| | ctacacgaaattcatatgggagaagtaccgcaagctgtcgccgacggccc | |
| | gacggatgttcgactatttcagctcgcaccgggagccgtacccgctcaag | |
| | ctggaaaccttccgcctcatgtgcggatcggattccacccgcgtgaagaa | |
| | gtggcgcgagcaggtcggcgaagcctgcgaagagttgcgaggcagcggcc | |
| | tggtggaacacgcctgggtcaatgatgacctggtgcattgcaaacgctag | |
| | ggccttgtggggtcagttccggctgggggttcagcagcccctgctcggat | |
| | ctgttggaccggacagtagtcatggttgatgggctgcctgtatcgagtgg | |
| | tgattttgtgccgagctgccggtcggggagctgttggctggctggtggca | |
| | ggatatattgtggtgtaaacaaattgacgcttagacaacttaataacaca | |
| | ttgcggacgtttttaatgtactggggttgaacactct | |
| 70 | gacgtcgttgtggttggtgctttccttacattctgagcctctttccttct | pU10::gR |
| | aatccactcatctgcatcttcttgtgtccttactaatacctcattggttc | NAs of |
| | caaattccctccctttaagcaccagctcgtttctgttcttccacagcctc | BPEp_a |
| | ccaagtatccaagggactaaagcctccacattcttcagatcaggatattc | and |
| | ttgtttaagatgttgaactctatggaggtttgtatgaactgatgatctag | BPEp_b. |
| | gaccggataagttcccttcttcatagcgaacttattcaaagaatgttttg | Same as |
| | tgtatcattcttgttacattgttattaatgaaaaaatattattggtcatt | SEQ ID |
| | ggactgaacacgagtgttaaatatggaccaggccccaaataagatccatt | NO: 74 |
| | gatatatgaattaaataacaagaataaatcgagtcaccaaaccacttgcc | which has |
| | ttttttaacgagacttgttcaccaacttgatacaaaagtcattatcctat | no |
| | gcaaatcaataatcatacaaaaatatccaataacactaaaaaaattaaaa | promoter |
| | gaaatggataatttcacaatatgttatacgataaagaagttacttttcca | |
| | agaaattcactgattttataagcccacttgcattagataaatggcaaaaa | |
| | aaaacaaaaaggaaaagaaataaagcacgaagaattctagaaaatacgaa | |
| | atacgcttcaatgcagtgggacccacggttcaattattgccaattttcag | |
| | ctccaccgtatatttaaaaaataaaacgataatgctaaaaaaatataaat | |
| | cgtaacgatcgttaaatctcaacggctggatcttatgacgaccgttaagg | |
| | aaattgtggttgtcggacgaagtccagtaataaacggcgtcaaagtggtt | |
| | gcagccggcacacacgagtcgtgtttatcaactcaaagcacaaatacttt | |
| | tcctcaacctaaaaataaggcaattagccaaaaacaactttgcgtgtaaa | |
| | caacgctcaatacacgtgtcattttattattagctattgcttcaccgcct | |
| | tagctttctcgtgacctagtcgtcctcgtcttttcttcttcttcttctat | |
| | aaaacaatacccaaagagctcttcttcttcacaattcagatttcaatttc | |
| | tcaaaatcttaaaaactttctctcaattctctctaccgtgatcaaggtaa | |
| | atttctgtgttccttattctctcaaaatcttcgattttgttttcgttcga | |
| | tcccaatttcgtatatgttctttggtttagattctgttaatcttagatcg | |
| | tagacgattttctgggtttgatcgttagatatcatcttaattctcgatta | |
| | gggtttcatagatatcatccgatttgttcaaataatttgagttttgtcga | |
| | ataattactcttcgatttgtgatttctatctagatctggtgttagtttct | |
| | agtttgtgcgatcgaatttgtcgattaatctgagtttttctgattaacag | |
| | atgcagatcttaatgacaaagcaccagtggtctagtggtagaatagtacc | |
| | ctgccacggtacagacccgggttcgattcccggctggtgcatggagaatg | |
| | gtgcgcgcagtgttttagagctagaaatagcaagttaaaataaggctagt | |
| | ccgttatcaacttgaaaaagtggcaccgagtcggtgcaacaaagcaccag | |
| | tggtctagtggtagaatagtaccctgccacggtacagacccgggttcgat | |
| | tcccggctggtgcaaagaggagtgacttggttttgttttagagctagaaa | |
| | tagcaagttaaaataaggctagtccgttatcaacttgaaaaagtggcacc | |
| | gagtcggtgcaacaaagcaccagtggtctagtggtagaatagtaccctgc | |
| | cacggtacagacccgggttcgattcccggctggtgcaactctctctacaa | |
| | gtggtaagttttagagctagaaatagcaagttaaaataaggctagtccgt | |
| | tatcaacttgaaaaagtggcaccgagtcggtgcaacaaagcaccagtggt | |
| | ctagtggtagaatagtaccctgccacggtacagacccgggttcgattccc | |
| | ggctggtgcacattagatgagtgatataaagttttagagctagaaatagc | |
| | aagttaaaataaggctagtccgttatcaacttgaaaaagtggcaccgagt | |
| | cggtgcaacaaagcaccagtggtctagtggtagaatagtaccctgccacg | |
| | gtacagacccgggttcgattcccggctggtgcaagtttccgtgaatcaaa | |
| | aatgttttagagctagaaatagcaagttaaaataaggctagtccgttatc | |
| | aacttgaaaaagtggcaccgagtcggtgcaacaaagcaccagtggtctag | |
| | tggtagaatagtaccctgccacggtacagacccgggttcgattcccggct | |
| | ggtgcacacatgttctattgtaagatgttttagagctagaaatagcaagt | |
| | taaaataaggctagtccgttatcaacttgaaaaagtggcaccgagtcggt | |
| | gccaacaaagcaccagtggtctagtggtagaatagtaccctgccacggta | |
| | cagacccgggttcgattcccggctggtgca | |
| 71 | gacgtcgttgtggttggtgctttccttacattctgagcctctttccttct | pU10::gRNAs |
| | aatccactcatctgcatcttcttgtgtccttactaatacctcattggttc | 1a-3a |
| | caaattccctccctttaagcaccagctcgtttctgttcttccacagcctc | for SUC9a |
| | ccaagtatccaagggactaaagcctccacattcttcagatcaggatattc | Same as |
| | ttgtttaagatgttgaactctatggaggtttgtatgaactgatgatctag | SEQ ID |
| | gaccggataagttcccttcttcatagcgaacttattcaaagaatgttttg | NO: 77 |
| | tgtatcattcttgttacattgttattaatgaaaaaatattattggtcatt | which has |
| | ggactgaacacgagtgttaaatatggaccaggccccaaataagatccatt | no |
| | gatatatgaattaaataacaagaataaatcgagtcaccaaaccacttgcc | promoter |
| | ttttttaacgagacttgttcaccaacttgatacaaaagtcattatcctat | |
| | gcaaatcaataatcatacaaaaatatccaataacactaaaaaaattaaaa | |
| | gaaatggataatttcacaatatgttatacgataaagaagttacttttcca | |
| | agaaattcactgattttataagcccacttgcattagataaatggcaaaaa | |
| | aaaacaaaaaggaaaagaaataaagcacgaagaattctagaaaatacgaa | |
| | atacgcttcaatgcagtgggacccacggttcaattattgccaattttcag | |
| | ctccaccgtatatttaaaaaataaaacgataatgctaaaaaaatataaat | |
| | cgtaacgatcgttaaatctcaacggctggatcttatgacgaccgttaagg | |
| | aaattgtggttgtcggacgaagtccagtaataaacggcgtcaaagtggtt | |
| | gcagccggcacacacgagtcgtgtttatcaactcaaagcacaaatacttt | |
| | tcctcaacctaaaaataaggcaattagccaaaaacaactttgcgtgtaaa | |
| | caacgctcaatacacgtgtcattttattattagctattgcttcaccgcct | |
| | tagctttctcgtgacctagtcgtcctcgtcttttcttcttcttcttctat | |
| | aaaacaatacccaaagagctcttcttcttcacaattcagatttcaatttc | |
| | tcaaaatcttaaaaactttctctcaattctctctaccgtgatcaaggtaa | |
| | atttctgtgttccttattctctcaaaatcttcgattttgttttcgttcga | |
| | tcccaatttcgtatatgttctttggtttagattctgttaatcttagatcg | |
| | tagacgattttctgggtttgatcgttagatatcatcttaattctcgatta | |
| | gggtttcatagatatcatccgatttgttcaaataatttgagttttgtcga | |
| | ataattactcttcgatttgtgatttctatctagatctggtgttagtttct | |
| | agtttgtgcgatcgaatttgtcgattaatctgagtttttctgattaacag | |
| | atgcagatcttaatgacaaagcaccagtggtctagtggtagaatagtacc | |
| | ctgccacggtacagacccgggttcgattcccggctggtgcatatcgtata | |
| | gtaattagctggttttagagctagaaatagcaagttaaaataaggctagt | |
| | ccgttatcaacttgaaaaagtggcaccgagtcggtgcaacaaagcaccag | |
| | tggtctagtggtagaatagtaccctgccacggtacagacccgggttcgat | |
| | tcccggctggtgcacatcatttacgtaaacgtttgttttagagctagaaa | |
| | tagcaagttaaaataaggctagtccgttatcaacttgaaaaagtggcacc | |
| | gagtcggtgcaacaaagcaccagtggtctagtggtagaatagtaccctgc | |
| | cacggtacagacccgggttcgattcccggctggtgcaagggttgaagagt | |
| | ttatatagttttagagctagaaatagcaagttaaaataaggctagtccgt | |
| | tatcaacttgaaaaagtggcaccgagtcggtgccaacaaagcaccagtgg | |
| | tctagtggtagaatagtaccctgccacggtacagacccgggttcgattcc | |
| | cggctggtgca | |
| 72 | gacgtcgttgtggttggtgctttccttacattctgagcctctttccttct | pU10::gR |
| | aatccactcatctgcatcttcttgtgtccttactaatacctcattggttc | NAs 1b-3b |
| | caaattccctccctttaagcaccagctcgtttctgttcttccacagcctc | for SUC9b |
| | ccaagtatccaagggactaaagcctccacattcttcagatcaggatattc | Same as |
| | ttgtttaagatgttgaactctatggaggtttgtatgaactgatgatctag | SEQ ID |
| | gaccggataagttcccttcttcatagcgaacttattcaaagaatgttttg | NO: 78 |
| | tgtatcattcttgttacattgttattaatgaaaaaatattattggtcatt | which has |
| | ggactgaacacgagtgttaaatatggaccaggccccaaataagatccatt | no |
| | gatatatgaattaaataacaagaataaatcgagtcaccaaaccacttgcc | promoter |
| | ttttttaacgagacttgttcaccaacttgatacaaaagtcattatcctat | |
| | gcaaatcaataatcatacaaaaatatccaataacactaaaaaaattaaaa | |
| | gaaatggataatttcacaatatgttatacgataaagaagttacttttcca | |
| | agaaattcactgattttataagcccacttgcattagataaatggcaaaaa | |
| | aaaacaaaaaggaaaagaaataaagcacgaagaattctagaaaatacgaa | |
| | atacgcttcaatgcagtgggacccacggttcaattattgccaattttcag | |
| | ctccaccgtatatttaaaaaataaaacgataatgctaaaaaaatataaat | |
| | cgtaacgatcgttaaatctcaacggctggatcttatgacgaccgttaagg | |
| | aaattgtggttgtcggacgaagtccagtaataaacggcgtcaaagtggtt | |
| | gcagccggcacacacgagtcgtgtttatcaactcaaagcacaaatacttt | |
| | tcctcaacctaaaaataaggcaattagccaaaaacaactttgcgtgtaaa | |
| | caacgctcaatacacgtgtcattttattattagctattgcttcaccgcct | |
| | tagctttctcgtgacctagtcgtcctcgtcttttcttcttcttcttctat | |
| | aaaacaatacccaaagagctcttcttcttcacaattcagatttcaatttc | |
| | tcaaaatcttaaaaactttctctcaattctctctaccgtgatcaaggtaa | |
| | atttctgtgttccttattctctcaaaatcttcgattttgttttcgttcga | |
| | tcccaatttcgtatatgttctttggtttagattctgttaatcttagatcg | |
| | tagacgattttctgggtttgatcgttagatatcatcttaattctcgatta | |
| | gggtttcatagatatcatccgatttgttcaaataatttgagttttgtcga | |
| | ataattactcttcgatttgtgatttctatctagatctggtgttagtttct | |
| | agtttgtgcgatcgaatttgtcgattaatctgagtttttctgattaacag | |
| | atgcagatcttaatgacaaagcaccagtggtctagtggtagaatagtacc | |
| | ctgccacggtacagacccgggttcgattcccggctggtgcatacgttgca | |
| | tgtgttctttagttttagagctagaaatagcaagttaaaataaggctagt | |
| | ccgttatcaacttgaaaaagtggcaccgagtcggtgcaacaaagcaccag | |
| | tggtctagtggtagaatagtaccctgccacggtacagacccgggttcgat | |
| | tcccggctggtgcaggagtgtgtgtaaatgtgaggttttagagctagaaa | |
| | tagcaagttaaaataaggctagtccgttatcaacttgaaaaagtggcacc | |
| | gagtcggtgcaacaaagcaccagtggtctagtggtagaatagtaccctgc | |
| | cacggtacagacccgggttcgattcccggctggtgcacctaattctaacc | |
| | gatattggttttagagctagaaatagcaagttaaaataaggctagtccgt | |
| | tatcaacttgaaaaagtggcaccgagtcggtgccaacaaagcaccagtgg | |
| | tctagtggtagaatagtaccctgccacggtacagacccgggttcgattcc | |
| | cggctggtgca | |
| 73 | cacatgtcacactctggtgcacatggtgttggtagaaagaccaaatgtca | pW1::W1. |
| | cgcccataactatcaatcgttgtcagtattgctatattccttggggattt | expression |
| | acgactcattgacaaccagagtggttttaagacaactgtttttatctagc | construct |
| | tatggaagattctcacgatgctcaagaatttgaagtgatcaatggggttt | This |
| | gatgcaagaggtaatgtcctgagttggagatttgagttattacgactcgt | construct |
| | ataggtcattgaggattgaggagatcacaccgatatgatgcccgaggagg | in a |
| | aggaagtgcatcatgagcttgtacgagatttagagccttcatcttaggag | plasmid is |
| | gaggattgattaccttcctgtgtttataccttaggcgtaattcaatactt | SEQ ID |
| | ttagtcgggtagtgatctgctctgaccctatatccttttttatatttgat | NO: 103 |
| | gtatttgaggggtgagatcttcattgaaactgttttctttatgtaacaga | |
| | tgtgtatcgataatttaattacttttacactttgggttgtatccaaattt | |
| | cttctattgtgacactatttatttgatttatttacatataaaggtttcat | |
| | attataaatttgcgttcatttatttacatatttacttacgacgtgaaaac | |
| | ttgatgtttcttaagcaatgcatttattttattttatcttacttatacaa | |
| | aaaaaatgatgagatatttttttaacttaaaaatagaaattaaaagttaa | |
| | tgacacgtattccacaatatttattattcaaaccattttatgtaatattt | |
| | aaaaaaaaagtatatgagatgttacataaagtgaccataagttggaatac | |
| | ctcccactaacaaaaagtaacaaataaattactaggattggccttaaaaa | |
| | aatcgcgagttgttaattaatttactccttaatattataatagtcaatta | |
| | attaaattggtccctaaaattaattgttttcttaaaaaaaataagtaaaa | |
| | catgagaatccaaatcaattttagttggtggttggtttgagtattttttt | |
| | ttcatttttaaagaaaaataaaataaaagtgaaaatacattttcttaaaa | |
| | tttagaaaatatacataaaaacagatcaatagtaaagcaacgttttcaat | |
| | cttttcttaagaagtaaaaatacggtgacactttgacattttttctcaat | |
| | atattttttaaatcttaaaaatttaaaataaaaaatatttttaaaaaata | |
| | aaaataaaaataaaaaataaacactcaaactaaaataagtaaaagtttta | |
| | attttcacaatgtggcgcattaagactctcgaaatgctatttccaaaaag | |
| | aatggtagctgactagatgcaatctctcgtaggcgttaggttggcaatgg | |
| | aaggtttggttgcacgcttgcacgccccatgtcagctgataacctacttt | |
| | cttagagtaacccactttccatattcacaaatgattaatggtcaatcttc | |
| | taattcacgcactactcaccccaaccatccattaggatattccgagtcta | |
| | atggctccattattaaaaaaaaaaaatctttttatttaaattttaactit | |
| | tcatttttatatatttattcttaaacgaaaaatctaaactactatatttg | |
| | attcatattctttatttattttttaaaatttccccattgtttaaattttt | |
| | taaatcaaaatcaaaatcatttaagtttacttaattttaatattgtatga | |
| | aataattaaaattaaaaaaaaatgtttttttaacaaagtaaaaagaaatt | |
| | ataatggtgatagaaccacgggaaagtatttgtgcaaaataattgaaatt | |
| | acaagtattattttctattacaatttttttaattgtttctttgctttttt | |
| | ttttttttaacttctattttgcaagcatgtattttatatttaatgtgtgt | |
| | ggagggggacggagtacaaactcttttttgtctatattcagtaaaatgtt | |
| | ggtggtgacgtgccttgcgtgatgaactaccatttcaaacaccacacgac | |
| | accacacatccattttaaatataaccccatcatagatatcccgaatcatc | |
| | aaattattacttcatagcaactagcaaattaattagcttcaccaatggac | |
| | tcattgttacttctaaaagaaattgccacttccattttgatcttcttgat | |
| | cactcgcctctccattcaaacattcctcaaaagctatcgccagaaactcc | |
| | caccggggccaaaagggtggccagttgtgggtgcactccctctcatggga | |
| | agcatgcctcatgtcaccttagcaaagatggcaaaaaaatatggacctat | |
| | aatgtacctcaaaatgggcactaacaacatggttgtggcctctactccag | |
| | ctgctgctcgtgccttcctcaaaacccttgatcaaaacttttcaaaccgg | |
| | ccctccaatgctggtgcaacccatttggcttatgatgcacgggatatggt | |
| | gtttgctcattacggatcacggtggaagttgctaagaaaactaagtaact | |
| | tgcacatgcttggaggaaaggcacttgatgattgggcccaaattcgagat | |
| | gaagagatggggcacatgcttggtgcaatgtacgattgtaacaagaggga | |
| | tgaggctgtggtggtggcggagatgttgacatattcaatggccaacatga | |
| | ttggccaagttatattgagtcgtcgagtgtttgagacaaagggttcggag | |
| | tctaacgagttcaaggacatggtggttgagctcatgaccgttgctggtta | |
| | cttcaacattggtgacttcataccctttttggccaagttggacttgcaag | |
| | gcatagagcgtggcatgaagaagttgcacaagaagtttgatgcgttgtta | |
| | acgagcatgattgaggagcatgttgcttctagtcacaagagaaagggcaa | |
| | gcccgatttcttagacatggtaatggctcatcatagtgagaactccgatg | |
| | gggaggaactatcgctcaccaacatcaaggcactactcttgaacctattc | |
| | accgcaggcaccgatacatcttcaagtataatagagtggtccttagccga | |
| | gatgttgaagaagcccagcataatgaagaaggctcatgaagaaatggacc | |
| | aagtcataggaagggatcgccgcctcaaagaatctgacataccaaagctt | |
| | ccctacttccaagccatttgcaaagaaacctatagaaagcacccttcaac | |
| | acccctaaacctgcctcgaatctcatctgaaccgtgccaagtgaatggtt | |
| | actacattcccgagaacactaggctgaatgtgaacatttgggccatagga | |
| | agagatcctgatgtgtggaacaatcctttggagtttatgcccgagaggtt | |
| | tttgagtgggaagaatgccaaaattgacccacgtgggaatgattttgagc | |
| | ttattccatttggtgctgggaggaggatttgtgcagggactaggatgggg | |
| | attgtgttggttcactacattttgggcactttggtgcattcgtttgattg | |
| | gaagctacccaatggggtgagggagttagacatggaggagtcctttgggc | |
| | ttgccttgcaaaaaaaggttccacttgctgctttggttacccctaggttg | |
| | aacccaagtgcttacatttcttag | |
| 74 | caaagcaccagtggtctagtggtagaatagtaccctgccacggtacagac | gRNAs of |
| | ccgggttcgattcccggctggtgcatggagaatggtgcgcgcagtgtttt | BPEp |
| | agagctagaaatagcaagttaaaataaggctagtccgttatcaacttgaa | construct |
| | aaagtggcaccgagtcggtgcaacaaagcaccagtggtctagtggtagaa | (with |
| | tagtaccctgccacggtacagacccgggttcgattcccggctggtgcaaa | tRNA, no |
| | gaggagtgacttggttttgttttagagctagaaatagcaagttaaaataa | promoter) |
| | ggctagtccgttatcaacttgaaaaagtggcaccgagtcggtgcaacaaa | Same as |
| | gcaccagtggtctagtggtagaatagtaccctgccacggtacagacccgg | SEQ ID |
| | gttcgattcccggctggtgcaactctctctacaagtggtaagttttagag | NO: 70 |
| | ctagaaatagcaagttaaaataaggctagtccgttatcaacttgaaaaag | which has |
| | tggcaccgagtcggtgcaacaaagcaccagtggtctagtggtagaatagt | a |
| | accctgccacggtacagacccgggttcgattcccggctggtgcacattag | promoter |
| | atgagtgatataaagttttagagctagaaatagcaagttaaaataaggct | |
| | agtccgttatcaacttgaaaaagtggcaccgagtcggtgcaacaaagcac | |
| | cagtggtctagtggtagaatagtaccctgccacggtacagacccgggttc | |
| | gattcccggctggtgcaagtttccgtgaatcaaaaatgttttagagctag | |
| | aaatagcaagttaaaataaggctagtccgttatcaacttgaaaaagtggc | |
| | accgagtcggtgcaacaaagcaccagtggtctagtggtagaatagtaccc | |
| | tgccacggtacagacccgggttcgattcccggctggtgcacacatgttct | |
| | attgtaagatgttttagagctagaaatagcaagttaaaataaggctagtc | |
| | cgttatcaacttgaaaaagtggcaccgagtcggtgccaacaaagcaccag | |
| | tggtctagtggtagaatagtaccctgccacggtacagacccgggttcgat | |
| | tcccggctggtgca | |
| 75 | caaagcaccagtggtctagtggtagaatagtaccctgccacggtacagac | gRNAs |
| | ccgggttcgattcccggctggtgcaaaaaaatgtgaatgtggatggtttt | 1a-3a of |
| | agagctagaaatagcaagttaaaataaggctagtccgttatcaacttgaa | ANTa with |
| | aaagtggcaccgagtcggtgcaacaaagcaccagtggtctagtggtagaa | the tRNAs |
| | tagtaccctgccacggtacagacccgggttcgattcccggctggtgcaaa | (no |
| | atttataacatttacatcgttttagagctagaaatagcaagttaaaataa | promoter) |
| | ggctagtccgttatcaacttgaaaaagtggcaccgagtcggtgcaacaaa | Same as |
| | gcaccagtggtctagtggtagaatagtaccctgccacggtacagacccgg | SEQ ID |
| | gttcgattcccggctggtgcatgatgtctttagaccagacagttttagag | NO: 65 |
| | ctagaaatagcaagttaaaataaggctagtccgttatcaacttgaaaaag | which has |
| | tggcaccgagtcggtgccaacaaagcaccagtggtctagtggtagaatag | a |
| | taccctgccacggtacagacccgggttcgattcccggctggtgca | promoter |
| 76 | caaagcaccagtggtctagtggtagaatagtaccctgccacggtacagac | gRNAs |
| | ccgggttcgattcccggctggtgcattttgtatctctctatatctgtttt | 1b-3b; |
| | agagctagaaatagcaagttaaaataaggctagtccgttatcaacttgaa | (with |
| | aaagtggcaccgagtcggtgcaacaaagcaccagtggtctagtggtagaa | tRNAs) of |
| | tagtaccctgccacggtacagacccgggttcgattcccggctggtgcatt | ANTb in |
| | tctttgtgtctcagctcagttttagagctagaaatagcaagttaaaataa | the |
| | ggctagtccgttatcaacttgaaaaagtggcaccgagtcggtgcaacaaa | construct |
| | gcaccagtggtctagtggtagaatagtaccctgccacggtacagacccgg | (no |
| | gttcgattcccggctggtgcatgggaaaagattcagaactcgttttagag | promoter) |
| | ctagaaatagcaagttaaaataaggctagtccgttatcaacttgaaaaag | Same as |
| | tggcaccgagtcggtgccaacaaagcaccagtggtctagtggtagaatag | SEQ ID |
| | taccctgccacggtacagacccgggttcgattcccggctggtgca | NO: 67 |
| | | which has |
| | | a |
| | | promoter |
| 77 | caaagcaccagtggtctagtggtagaatagtaccctgccacggtacagac | gRNAs |
| | cgggttcgattcccggctggtgcatatcgtatagtaattagctggtttta | 1a-3a of |
| | gagctagaaatagcaagttaaaataaggctagtccgttatcaacttgaaa | SUC9a |
| | aagtggcaccgagtcggtgcaacaaagcaccagtggtctagtggtagaat | with the |
| | agtaccctgccacggtacagacccgggttcgattcccggctggtgcacat | tRNAs (no |
| | catttacgtaaacgtttgttttagagctagaaatagcaagttaaaataag | promoter) |
| | gctagtccgttatcaacttgaaaaagtggcaccgagtcggtgcaacaaag | Same as |
| | caccagtggtctagtggtagaatagtaccctgccacggtacagacccggg | SEQ ID |
| | ttcgattcccggctggtgcaagggttgaagagtttatatagttttagagc | NO: 71 |
| | tagaaatagcaagttaaaataaggctagtccgttatcaacttgaaaaagt | which has |
| | ggcaccgagtcggtgccaacaaagcaccagtggtctagtggtagaatagt | a |
| | accctgccacggtacagacccgggttcgattcccggctggtgca | promoter |
| 78 | caaagcaccagtggtctagtggtagaatagtaccctgccacggtacagac | gRNAs |
| | ccgggttcgattcccggctggtgcatacgttgcatgtgttctttagtttt | 1b-3b; |
| | agagctagaaatagcaagttaaaataaggctagtccgttatcaacttgaa | (with |
| | aaagtggcaccgagtcggtgcaacaaagcaccagtggtctagtggtagaa | tRNAs of |
| | tagtaccctgccacggtacagacccgggttcgattcccggctggtgcagg | SUC9b in |
| | agtgtgtgtaaatgtgaggttttagagctagaaatagcaagttaaaataa | the |
| | ggctagtccgttatcaacttgaaaaagtggcaccgagtcggtgcaacaaa | construct) |
| | gcaccagtggtctagtggtagaatagtaccctgccacggtacagacccgg | Same as |
| | gttcgattcccggctggtgcacctaattctaaccgatattggttttagag | SEQ ID |
| | ctagaaatagcaagttaaaataaggctagtccgttatcaacttgaaaaag | NO: 72 |
| | tggcaccgagtcggtgccaacaaagcaccagtggtctagtggtagaatag | which has |
| | taccctgccacggtacagacccgggttcgattcccggctggtgca | a |
| | | promoter |
| 79-88 | | Primers of |
| | | Table 1 |
| 89-98 | | Primers of |
| | | Table 2 |
| 99 | gtgccgaattcggatccagcgatctggattttagtactggattttggttt | RC4269 |
| | taggaattagaaattttattgatagaagtattttacaaatacaaatacat | M. Frank |
| | actaagggtttcttatatgctcaacacatgagcgaaaccctataggaacc | AP3-P5U |
| | ctaattcccttatctgggaactactcacacattattatggagaaactcga | dCas9-TV |
| | gcttgtcgatcgactctagctagagaagctcactttccaacaaccttagt | CONTROL |
| | aatttctcccttaacataatgaacaaattcttcaagttgatcagctctag | Construct |
| | aagcaagtctatcttcttcttgtccaagataagcttgtctagcttcaaga | (RCGG30 |
| | ataactggttgatattgagctggaagtctttccatagcccaatcagcagc | 02) |
| | aacatcctttggagcaatctttccagtaacagcagaataccaaattctag | Plasmid |
| | aaagagtaagaacaacatttctttcatctccagcccaatctggtggagaa | comprising |
| | ttccaaagagtaagagtttcattaagagcttcaaaaagatcttgttctgg | expression |
| | aactggatcaaaaagttcttcagcagctggtccaacaagagcaacagaat | construct |
| | gttctctagccttagtaagaagaatagcaagatcaatatcaatagtagct | of SEQ ID |
| | ggttcaaaaattccagcaagaatatcatttctttgccattctccaaattg | NO: 62 |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagtgttcaaaa | |
| | ataaaaagtgattttgagatataatatacatacgacaattaatacaatat | |
| | tatttttgtgtctatccctctcttagaccctattatttatcacatttgaa | |
| | attcagaattttctttttctctcgttgttgtacaaatatctaatcgttaa | |
| | ttattattaatgcattaaatatttaaaatataattaatgatattaatttt | |
| | cctagcgtaagactaataataaatatcgacattatgcgagattatgacat | |
| | gatgttatgttaatagtacagaataaagtaaaaaaatatatataacaata | |
| | ataatttaaaatatttaaaatgacatataaagtagttttctaaaacttca | |
| | gtgattagttcaatttattatttcgcaacaattatgatggttaattaagg | |
| | tgaattagaagaagaactgaaaattaagataattaaggagaacaaatgag | |
| | cacttcaaaatttgctattaagccgactctgctagccatgcatgttggat | |
| | gtaacttcacccgattgatgggtccttaattataatttttcttatgataa | |
| | aaaataaattgctactaaggcgtactgaactattcgaaatgacaacacta | |
| | caaggcctcgacaacgttaacttttttccaatttagtggggcgttacaag | |
| | ctaaagcttttttatttttgaactttaaattgctctttcactttcattta | |
| | ataacgcctaaacctagctacgagcaatataacctgggtgtatatagcta | |
| | tatatatcatatgcagagcaatctgtaaccccaaggataaaacgttgtgt | |
| | tgcttattatagaaatcacagttttaaatggaacgagtgacagagtttgt | |
| | ttgtttctaaataggataggaggcatttaatcggaaattggacaggagaa | |
| | agttgttttctttctgcaaattatgcctccagaacttgttttacggtaag | |
| | aaaatttacaccaaatttacaagtattggttctgaacgctacttgcggaa | |
| | attttatgccgccagtgacttcttggcactttaaataagttaattagtta | |
| | atcatttgaaaatatattaacaagaagaatttttttctagtgtaatcaag | |
| | aggtattttaagcttctatcttacactcaattaatcttctattctcctat | |
| | tgattggtctattttaaagtaaagtaatatattcaattacgacttcatgc | |
| | taaattcaaagattataacagtaaaaacaatttttattcacctctattca | |
| | aactgccagtttagcgcaagtgtttatttttctgataaacttgcggccgc | |
| | acaaaacccagcattgcttcaatgtgcataagcatgggtacggtggtggt | |
| | gtttctccaacctccatttcagattgtcttgatttgtaattcaaacacac | |
| | acttagtcttaaaatcatattttctcatatctcaattgtgatttttccaa | |
| | atgcgttgtgtgtaggcgaagctaagtttaggttattgctcaagtgccac | |
| | aagtttgagagggttttactgtcttttttatttaatctagaaatatgatt | |
| | tttggacacgtctacttgttacattccgtttacttatggtagtatttgac | |
| | taataaagacattaagattatagtagcatttgactaataaagacacgcgt | |
| | acgtaggtcactattttattggctaagtaaaaaaaaaaaaaaacacgaaa | |
| | aaactttcaaacaaaggggaagcggagaaatcagcctaacttgatattag | |
| | ctgtacaaaatacataaatagctgtcggaaataattatctcttgcagtat | |
| | atatatagaaagtagcagactctaaaatataacatgaagatgtgattaag | |
| | catttattccattatatggcccataaacatgcatgatgcaaatagaagga | |
| | tgtagggtaaagttgccctaagaaacgaattgaactgcggtcgttgcggt | |
| | atgagaaagagagagagaaaagtgagtgaaggaaggtgggagaatggttt | |
| | ccgtgacggagtaggacggaaagtaagggtccccacagtgcacatgggag | |
| | cggatttgacgatgagtcgagaaagagcacaagacgtttatgacaaagcc | |
| | tgcaatagctttcgttaatttggcaaaagacccctaccatttgcggcaat | |
| | gcacctccccgttctattatttctctttccctcattcattatatccccta | |
| | ttctctgccccagatcgatcgaacaagtgaattagctagaagcattgaag | |
| | gacgtagctgctccatggattacaaagatgacgatgataaagactacaag | |
| | gacgacgacgataaaatggcccctaaaaagaagcgcaaggtgggtattca | |
| | tggggtgcctgcagctggaatggataagaagtactccatoggactggcta | |
| | tcggaactaactccgtgggatgggctgtgatcactgatgagtacaaggtg | |
| | ccatccaagaagttcaaggtgctgggaaacactgatagacactccatcaa | |
| | gaagaacctgatcggagctctgctgttcgattccggagagactgctgagg | |
| | ctactagactgaagagaactgctagaagaagatacactagaagaaagaac | |
| | agaatctgctacctgcaagagatcttctccaacgagatggctaaggtgga | |
| | tgattccttcttccacagactggaggagtccttcctggtggaggaggata | |
| | agaagcacgagagacacccaatcttcggaaacatcgtggatgaggtggct | |
| | taccacgagaagtacccaactatctaccacctgagaaagaagctggtgga | |
| | ttccactgataaggctgatctgagactgatctacctggctctggctcaca | |
| | tgatcaagttcagaggacacttcctgatcgagggagatctgaacccagat | |
| | aactccgatgtggataagctgttcatccaactggtgcaaacttacaacca | |
| | actgttcgaggagaacccaatcaacgcttccggagtggatgctaaggcta | |
| | tcctgtccgctagactgtccaagtccaggagactggagaacctgatcgct | |
| | caactgccaggagagaagaagaacggactgttcggaaacctgatcgctct | |
| | gtccctgggactgactccaaacttcaagtccaacttcgatctggctgagg | |
| | atgctaagctgcaactgtccaaggatacttacgatgatgatctggataac | |
| | ctgctggctcaaatcggagatcaatacgctgatctgttcctggctgctaa | |
| | gaacctgtccgatgctatcctgctgtccgatatcctgagagtgaacactg | |
| | agatcactaaggctccactgtccgcttccatgatcaagagatacgatgag | |
| | caccaccaagatctgactctgctgaaggctctggtgagacaacaactgcc | |
| | agagaagtacaaggagatcttcttcgatcaatccaagaacggatacgctg | |
| | gatacatcgatggaggagcttcccaagaggagttctacaagttcatcaag | |
| | ccaatcctggagaagatggatggaactgaggagctgctggtgaagctgaa | |
| | cagagaggatctgctgagaaagcaaagaactttcgataacggatccatcc | |
| | cacaccaaatccacctgggagagctgcacgctatcctgaggagacaagag | |
| | gatttctacccattcctgaaggataacagagagaagatcgagaagatcct | |
| | gactttcagaatcccatactacgtgggaccactggctagaggaaactcca | |
| | gattcgcttggatgactagaaagtccgaggagactatcactccatggaac | |
| | ttcgaggaggtaagtttctgcttctacctttgatatatatataataatta | |
| | tcattaattagtagtaatataatatttcaaatatttttttcaaaataaaa | |
| | gaatgtagtatatagcaattgcttttctgtagtttataagtgtgtatatt | |
| | ttaatttataacttttctaatatatgaccaaaatttgttgatgtgcaggt | |
| | ggtggataagggagcttccgctcaatccttcatcgagagaatgactaact | |
| | tcgataagaacctgccaaacgagaaggtgctgccaaagcactccctgctg | |
| | tacgagtacttcactgtgtacaacgagctgactaaggtgaagtacgtgac | |
| | tgagggaatgagaaagccagctttcctgtccggagagcaaaagaaggcta | |
| | tcgtggatctgctgttcaagactaacagaaaggtgactgtgaagcaactg | |
| | aaggaggattacttcaagaagatcgagtgcttcgattccgtggagatctc | |
| | cggagtggaggatagattcaacgcttccctgggaacttaccacgatctgc | |
| | tgaagatcatcaaggataaggatttcctggataacgaggagaacgaggat | |
| | atcctggaggatatcgtgctgactctgactctgttcgaggatagagagat | |
| | gatcgaggagagacttaagacttacgctcacctgttcgatgataaggtga | |
| | tgaagcaactgaagagaagaagatacactggatggggtagactgtccaga | |
| | aagctgatcaacggaatcagagataagcaatccggaaagactatcctgga | |
| | tttcctgaagtccgatggattcgctaacagaaacttcatgcaactgatcc | |
| | acgatgattccctgactttcaaggaggatatccaaaaggctcaagtgtcc | |
| | ggacaaggagattccctgcacgagcacatcgctaacctggctggatcccc | |
| | agctatcaagaagggaatcctgcaaactgtgaaggtggtggatgagctgg | |
| | tgaaggtgatgggtagacacaagccagagaacatcgtgatcgagatggct | |
| | agagagaaccaaactactcaaaagggacaaaagaactccagagagagaat | |
| | gaagagaatcgaggagggaatcaaggagctgggatcccaaatcctgaagg | |
| | agcacccagtggagaacactcaactgcaaaacgagaagctgtacctgtac | |
| | tacctgcaaaacggaagagatatgtacgtggatcaagagctggatatcaa | |
| | cagactgtccgattacgatgtggatgcaatcgtgccacaatccttcctga | |
| | aggatgattccatcgataacaaggtgctgactagatccgataagaacaga | |
| | ggaaagtccgataacgtgccatccgaggaggtggtgaagaagatgaagaa | |
| | ctactggagacaactgctgaacgctaagctgatcactcaaagaaagttcg | |
| | ataacctgactaaggctgagagaggaggactgtccgagctggataaggct | |
| | ggattcatcaagagacaactggtggagactagacaaatcactaagcacgt | |
| | ggctcaaatcctggattccagaatgaacactaagtacgatgagaacgata | |
| | agctgatcagagaggtgaaggtgatcactctgaagtccaagctggtgtcc | |
| | gatttcagaaaggatttccaattctacaaggtgagagagatcaacaacta | |
| | ccaccacgctcacgatgcttacctgaacgctgtggtgggaactgctctga | |
| | tcaagaagtacccaaagctggagtccgagttcgtgtacggagattacaag | |
| | gtgtacgatgtgagaaagatgatcgctaagtccgagcaagagatcggaaa | |
| | ggctactgctaagtacttcttctactccaacatcatgaacttcttcaaga | |
| | ctgagatcactctggctaacggagagatcagaaaaagaccactgatcgag | |
| | actaacggagagactggagagatcgtgtgggataagggaagagatttcgc | |
| | tactgtgagaaaggtgctgtccatgccacaagtgaacatcgtgaaaaaga | |
| | ctgaggtgcaaactggaggattctccaaggagtccatcctgccaaagaga | |
| | aactccgataagctgatcgctagaaagaaggattgggatccaaagaagta | |
| | cggaggattcgattccccaactgtggcttactccgtgctggtggtggcta | |
| | aggtggagaagggaaagtccaagaagctgaagtccgtgaaggagctgctg | |
| | ggaatcactatcatggagagatcctccttcgagaagaacccaatcgattt | |
| | cctggaggctaagggatacaaggaggtgaagaaggatctgatcatcaagc | |
| | tgccaaagtactccctgttcgagctggagaacggaagaaagagaatgctg | |
| | gcttccgctggagagctgcaaaagggaaacgagctggctctgccatccaa | |
| | gtacgtgaacttcctgtacctggcttcccactacgagaagctgaagggat | |
| | ccccagaggataacgagcaaaagcaactgttcgtggagcaacacaagcac | |
| | tacctggatgagatcatcgagcaaatctccgagttctccaagagagtgat | |
| | cctggctgatgctaacctggataaggtgctgtccgcttacaacaagcaca | |
| | gagataagccaatcagagagcaagctgagaacatcatccacctgttcact | |
| | ctgactaacctgggagctccagctgctttcaagtacttcgatactactat | |
| | cgatagaaagagatacacttccactaaggaggtgctggatgctactctga | |
| | tccaccaatccatcactggactgtacgagactagaatcgatctgtcccaa | |
| | ctgggaggagatagttcgaagaggcctgccgccacaaagaaagccggcca | |
| | ggcaaagaaaaaaaaacccaggggagggagtggtggactcctcgaccctg | |
| | gcactccaatggacgccgatctcgtagcatcatctaccgtcgtgtgggag | |
| | caggacgccgacccttttgctggcacagctgatgatttoccagcttttaa | |
| | tgaggaagaattggcttggctgatggagcttctcccacaaggggtagcgg | |
| | cgggctcctcgatcccggtactcccatggacgcagatcttgttgctagct | |
| | ccactgttgtatgggagcaggacgccgatcccttcgctggaaccgccgat | |
| | gatttcccagcattcaacgaggaagaacttgcatggctcatggagctctt | |
| | gccccaagctcgcggtggatctgggggtttgttggatcccggaaccccaa | |
| | tggacgcagacctcgtggcttctagtaccgtagtatgggaacaggacgct | |
| | gaccctttcgccggcaccgcagatgacttcccagcattcaacgaggagga | |
| | actggcctggctgatggaacttctgcctcagggtggatccggaggcctct | |
| | tggaccccggtacccctatggacgctgacctggtcgcatcttctaccgtg | |
| | gtttgggagcaagatgccgacccattcgcaggaacagctgatgacttccc | |
| | cgcattcaatgaagaagagctcgcatggctgatggaattgcttccacaag | |
| | ccagaggcggctctggagggcttcttgatcccggaacacccatggatgcc | |
| | gacctcgtggcctctagcaccgtggtatgggaacaagacgcagatccatt | |
| | cgcagggaccgcagacgatttccccgcttttaacgaggaggagcttgctt | |
| | ggctcatggagctgctgcctcagggtggatccgggggacttctegaccca | |
| | ggcacacccatggatgccgatctcgtcgccagttctaccgttgtctggga | |
| | gcaagacgcagaccctttcgccggaactgcagacgacttccctgccttta | |
| | atgaagaagaactggcttggctcatggaactgctcccccaggctagaggg | |
| | ggtagcggcgggggcggcagcggaggtgatgcacttgacgacttcgatct | |
| | tgatatgcttggcagcgacgcacttgatgacttcgatttggatatgctgg | |
| | ggtccgatgctcttgacgattttgatttggacatgttgggttcagacgca | |
| | ctcgatgatttcgatttggacatgctggcacgtggaagcgatgcccttga | |
| | tgactttgacctggatatgctcgggtccgatgctttggacgacttcgacc | |
| | tcgacatgttgggaagtgatgctcttgatgatttcgatcttgatatgctt | |
| | gggtccgacgctctcgatgattttgacctcgatatgctctgagcttgtca | |
| | agcagatcgttcaaacatttggcaataaagtttcttaagattgaatcctg | |
| | ttgccggtcttgcgatgattatcatataatttctgttgaattacgttaag | |
| | catgtaataattaacatgtaatgcatgacgttatttatgagatgggtttt | |
| | tatgattagagtcccgcaattatacatttaatacgcgatagaaaacaaaa | |
| | tatagcgcgcaaactaggataaattatcgcgcgcggtgtcatctatgtta | |
| | ctagatcgacgctttacgaattcccatggggagtgttcaaaaataaaaag | |
| | tgattttgagatataatatacatacgacaattaatacaatattatttttg | |
| | tgtctatccctctcttagaccctattatttatcacatttgaaattcagaa | |
| | ttttctttttctctcgttgttgtacaaatatctaatcgttaattattatt | |
| | aatgcattaaatatttaaaatataattaatgatattaattttcctagcgt | |
| | aagactaataataaatatcgacattatgcgagattatgacatgatgttat | |
| | gttaatagtacagaataaagtaaaaaaatatatataacaataataattta | |
| | aaatatttaaaatgacatataaagtagttttctaaaacttcagtgattag | |
| | ttcaatttattatttcgcaacaattatgatggttaattaaggtgaattag | |
| | aagaagaactgaaaattaagataattaaggagaacaaatgagcacttcaa | |
| | aatttgctattaagccgactctgctagccatgcatgttggatgtaacttc | |
| | acccgattgatgggtccttaattataatttttcttatgataaaaaataaa | |
| | ttgctactaaggcgtactgaactattcgaaatgacaacactacaaggcct | |
| | cgacaacgttaacttttttccaatttagtggggcgttacaagctaaagct | |
| | tttttatttttgaactttaaattgctctttcactttcatttaataacgcc | |
| | taaacctagctacgagcaatataacctgggtgtatatagctatatatatc | |
| | atatgcagagcaatctgtaaccccaaggataaaacgttgtgttgcttatt | |
| | atagaaatcacagttttaaatggaacgagtgacagagtttgtttgtttct | |
| | aaataggataggaggcatttaatcggaaattggacaggagaaagttgttt | |
| | tctttctgcaaattatgcctccagaacttgttttacggtaagaaaattta | |
| | caccaaatttacaagtattggttctgaacgctacttgcggaaattttatg | |
| | ccgccagtgacttcttggcactttaaataagttaattagttaatcatttg | |
| | aaaatatattaacaagaagaatttttttctagtgtaatcaagaggtattt | |
| | taagcttctatcttacactcaattaatcttctattctcctattgattggt | |
| | ctattttaaagtaaagtaatatattcaattacgacttcatgctaaattca | |
| | aagattataacagtaaaaacaatttttattcacctctattcaaactgcca | |
| | gtttagcgcaagtgtttatttttctgataaacttgcggccgcacaaaacc | |
| | cagcattgcttcaatgtgcataagcatgggtacggtggtggtgtttctcc | |
| | aacctccatttcagattgtcttgatttgtaattcaaacacacacttagtc | |
| | ttaaaatcatattttctcatatctcaattgtgatttttccaaatgcgttg | |
| | tgtgtaggcgaagctaagtttaggttattgctcaagtgccacaagtttga | |
| | gagggttttactgtcttttttatttaatctagaaatatgatttttggaca | |
| | cgtctacttgttacattccgtttacttatggtagtatttgactaataaag | |
| | acattaagattatagtagcatttgactaataaagacacgcgtacgtaggt | |
| | cactattttattggctaagtaaaaaaaaaaaaaaacacgaaaaaactttc | |
| | aaacaaaggggaagcggagaaatcagcctaacttgatattagctgtacaa | |
| | aatacataaatagctgtcggaaataattatctcttgcagtatatatatag | |
| | aaagtagcagactctaaaatataacatgaagatgtgattaagcatttatt | |
| | ccattatatggcccataaacatgcatgatgcaaatagaaggatgtagggt | |
| | aaagttgccctaagaaacgaattgaactgcggtcgttgcggtatgagaaa | |
| | gagagagagaaaagtgagtgaaggaaggtgggagaatggtttccgtgacg | |
| | gagtaggacggaaagtaagggtccccacagtgcacatgggagcggatttg | |
| | acgatgagtcgagaaagagcacaagacgtttatgacaaagcctgcaatag | |
| | ctttcgttaatttggcaaaagacccctaccatttgcggcaatgcacctcc | |
| | ccgttctattatttctotttccctcattcattatatcccctattctctgc | |
| | cccagatcgatcgaacaagtgaattagctagaagcattgaaggacgtagc | |
| | tgctaatgggtcagtcccttatgttacgtcctgtagaaaccccaacccgt | |
| | gaaatcaaaaaactcgacggcctgtgggcattcagtctggatcgcgaaaa | |
| | ctgtggaattgatcagcgttggtgggaaagcgcgttacaagaaagccggg | |
| | caattgctgtgccaggcagttttaacgatcagttcgccgatgcagatatt | |
| | cgtaattatgcgggcaacgtctggtatcagcgcgaagtctttataccgaa | |
| | aggtaagtcttactctctcttttttggtctgtatttttaattttttgaag | |
| | tatactatttgtactgacgctaataatcttttttcaggttgggcaggcca | |
| | gcgtatcgtgctgcgtttcgatgcggtcactcattacggcaaagtgtggg | |
| | tcaataatcaggaagtgatggagcatcagggcggctatacgccatttgaa | |
| | gccgatgtcacgccgtatgttattgccgggaaaagtgtacgtatcaccgt | |
| | ttgtgtgaacaacgaactgaactggcagactatcccgccgggaatggtga | |
| | ttaccgacgaaaacggcaagaaaaagcagtcttacttccatgatttcttt | |
| | aactatgccggaatccatcgcagcgtaatgctctacaccacgccgaacac | |
| | ctgggtggacgatatcaccgtggtgacgcatgtcgcgcaagactgtaacc | |
| | acgcgtctgttgactggcaggtacttcatgcttcaacgtgtaacttaaga | |
| | gatactgtgtgaaattttatatttccatacatttgcttgacctttgcttt | |
| | ttgtcaatttttttccccttacaggtggtggccaatggtgatgtcagcgt | |
| | tgaactgcgtgatgcggatcaacaggtggttgcaactggacaaggcacta | |
| | gcgggactttgcaagtggtgaatccgcacctctggcaaccgggtgaaggt | |
| | tatctctatgaactgtgcgtcacagccaaaagccagacagagtgtgatat | |
| | ctacccgcttcgcgtcggcatccggtcagtggcagtgaagggcgaacagt | |
| | tcctgattaaccacaaaccgttctactttactggctttggtcgtcatgaa | |
| | gatgcggacttgcgtggcaaaggattcgataacgtgctgatggtgcacga | |
| | ccacgcattaatggactggattggggccaactcctaccgtacctcgcatt | |
| | acccttacgctgaagagatgctcgactgggcagatgaacatggcatcgtg | |
| | gtgattgatgaaactgctgctgtcggctttaacctctctttaggcattgg | |
| | tttcgaagcgggcaacaagccgaaagaactgtacagcgaagaggcagtca | |
| | acggggaaactcagcaagcgcacttacaggcgattaaagagctgatagcg | |
| | cgtgacaaaaaccacccaagcgtggtgatgtggagtattgccaacgaacc | |
| | ggatacccgtccgcaaggtgcacgggaatatttcgcgccactggcggaag | |
| | caacgcgtaaactcgacccgacgcgtccgatcacctgcgtcaatgtaatg | |
| | ttctgcgacgctcacaccgataccatcagcgatctctttgatgtgctgtg | |
| | cctgaaccgttattacggatggtatgtccaaagcggcgatttggaaacgg | |
| | cagagaaggtactggaaaaagaacttctggcctggcaggagaaactgcat | |
| | cagccgattatcatcaccgaatacggcgtggatacgttagccgggctgca | |
| | ctcaatgtacaccgacatgtggagtgaagagtatcagtgtgcatggctgg | |
| | atatgtatcaccgcgtctttgatcgcgtcagcgccgtcgtcggtgaacag | |
| | gtatggaatttcgccgattttgcgacctcgcaaggcatattgcgcgttgg | |
| | cggtaacaagaaagggatcttcactcgcgaccgcaaaccgaagtcggcgg | |
| | cttttctgctgcaaaaacgctggactggcatgaacttcggtgaaaaaccg | |
| | cagcagggaggcaaacaaggttcggtgtccaagggcgaagaggtgattaa | |
| | agagttcatgaggttcaaagtgagaatggagggcagtatgaacggccatg | |
| | agttcgagatcgaaggtgaaggcgaaggtagaccatacgagggcacccag | |
| | acggcgaaactgaaagtgacgaagggcggcccactgcccttcgcctggga | |
| | cattctgagtccacaatttatgtacggttccaaggcatacgttaagcacc | |
| | ccgccgacatccctgattacaaaaagctgtcgttcccggaggggtttaaa | |
| | tgggaaagagtgatgaattttgaggacggagggctcgtcactgtgactca | |
| | agactcctcacttcaagacggcaccctaatttacaaggtgaagatgaggg | |
| | gaacaaatttcccccctgatggccctgttatgcagaaaaaaacaatggga | |
| | tgggaggcatccaccgagagactttacccgcgcgacggagtgctcaaagg | |
| | cgaaatccatcaggcactgaaactaaaggacggcggccactacctggttg | |
| | agttcaaaactatatatatggccaagaaacccgtccagttgccgggatat | |
| | tactatgtggacacaaaactggatataacgtcccacaacgaagattacac | |
| | catagtggaacagtatgagcgcagtgagggtcgtcaccatctattcctcg | |
| | gacatggcacgggttccacgggctcgggttccagcggcacggcaagctcg | |
| | gaggacaacaacatggcggtgatcaaagagttcatgcggtttaaagtccg | |
| | tatggagggcagcatgaatgggcacgaatttgaaattgagggagaaggcg | |
| | agggccgaccatatgaggggacacaaaccgcgaaactaaaagtcaccaaa | |
| | ggtgggccgcttccttttgcctgggacattctctctcctcagttcatgta | |
| | tggctccaaggcctacgttaaacatccggctgatatccccgattataaga | |
| | aattgtctttcccggaggggtttaaatgggagcgggttatgaatttcgag | |
| | gatggtggactggttactgttacacaagattcgagtctgcaggatggcac | |
| | attgatatacaaagtcaaaatgaggggtacgaattttcccccggacggcc | |
| | cggtgatgcagaaaaaaactatgggctgggaggccagcacggagaggctg | |
| | tacccgagggacggggttttgaaaggggagatacaccaggcgcttaaact | |
| | gaaggacggggggcactatctggtggaatttaagacgatatatatggcga | |
| | aaaagcccgtgcagctacctggctactactacgtcgataccaaactcgat | |
| | attacctctcataacgaggactacactatcgtggagcaatatgaaagatc | |
| | agaaggacgtcaccacctttttctatacggaatggacgagctttacaaag | |
| | cttgtgtgtcttgtcttatctggttcgtggtggtgagtttgttacaaaaa | |
| | aatctattttccctagttgagatgggaattgaactatctgttgttatgtg | |
| | gattttattttcttttttctctttagaaccttatggttgtgtcaagaagt | |
| | cttgtgtactttagttttatatctctgttttatctcttctattttcttta | |
| | ggatgcttgtgatgatgctgtttttttttgtccctaagcaaaaaaatatc | |
| | atattatatttggtccttggttcatttttttggtttttttttatcttcac | |
| | atataaatattgtttgaatatcttcaatcttttatttgtatgagacaatt | |
| | atttaagtatcgggtgacaatgcagctattatgtattgtcgattgttcgc | |
| | tcagagaggatgcacatgtgaccgagggacacgaagtgatccgtttaaac | |
| | tatcagtgtttgacaggatatattggcgggtaaacctaagagaaaagagc | |
| | gtttattagaataatcggatatttaaaagggcgtgaaaaggtttatccgt | |
| | tcgtccatttgtatgtgccagccgcctttgcgacgctcaccgggctggtt | |
| | gccctcgccgctgggctggcggccgtctatggccctgcaaacgcgccaga | |
| | aacgccgtcgaagccgtgtgcgagacaccgcggccgccggcgttgtggat | |
| | acctcgcggaaaacttggccctcactgacagatgaggggcggacgttgac | |
| | acttgaggggccgactcacccggcgcggcgttgacagatgaggggcaggc | |
| | tcgatttcggccggcgacgtggagctggccagcctcgcaaatcggcgaaa | |
| | acgcctgattttacgcgagtttcccacagatgatgtggacaagcctgggg | |
| | ataagtgccctgcggtattgacacttgaggggcgcgactactgacagatg | |
| | aggggcgcgatccttgacacttgaggggcagagtgctgacagatgagggg | |
| | cgcacctattgacatttgaggggctgtccacaggcagaaaatccagcatt | |
| | tgcaagggtttccgcccgtttttcggccaccgctaacctgtcttttaacc | |
| | tgcttttaaaccaatatttataaaccttgtttttaaccagggctgcgccc | |
| | tgtgcgcgtgaccgcgcacgccgaaggggggtgcccccccttctcgaacc | |
| | ctcccggcccgctaacgcgggcctcccatccccccaggggctgcgcccct | |
| | eggccgcgaacggcctcaccccaaaaatggcagcgctggccaattcccga | |
| | gtgcgcggaacccctatttgtttatttttctaaatacattcaaatatgta | |
| | tccgctcatgagacaataaccctgataaatgcttcaataatattgaaaaa | |
| | ggaagagtatggctaaaatgagaatatcaccggaattgaaaaaactgatc | |
| | gaaaaataccgctgcgtaaaagatacggaaggaatgtctcctgctaaggt | |
| | atataagctggtgggagaaaatgaaaacctatatttaaaaatgacggaca | |
| | gccggtataaagggaccacctatgatgtggaacgggaaaaggacatgatg | |
| | ctatggctggaaggaaagctgcctgttccaaaggtcctgcactttgaacg | |
| | gcatgatggctggagcaatctgctcatgagtgaggccgatggcgtccttt | |
| | gctcggaagagtatgaagatgaacaaagccctgaaaagattatcgagctg | |
| | tatgcggagtgcatcaggctctttcactccatcgacatatcggattgtcc | |
| | ctatacgaatagcttagacagccgcttagccgaattggattacttactga | |
| | ataacgatctggccgatgtggattgcgaaaactgggaagaggacactcca | |
| | tttaaagatccgcgcgagctgtatgattttttaaagacggaaaagcccga | |
| | agaggaacttgtcttttcccacggcgacctgggagacagcaacatctttg | |
| | tgaaagatggcaaagtaagtggctttattgatcttgggagaagcggcagg | |
| | gcggacaagtggtatgacattgccttctgcgtccggtcgctcagggagga | |
| | tatcggggaagaacagtatgtcgagctattttttgacttactggggatca | |
| | agcctgattgggagaaaataaaatattatattttactggatgaattgttt | |
| | tagctgtcagaccaagtttactcatatatactttagattgatttaaaact | |
| | tcattittaatttaaaaggatctaggtgaagatcctttttgataatctca | |
| | tgaccaaaatcccttaacgtgagttttcgttccactgagcgtcagacccc | |
| | gtagaaaagatcaaaggatcttcttgagatcctttttttctgcgcgtaat | |
| | ctgctgcttgcaaacaaaaaaaccaccgctaccagcggtggtttgtttgc | |
| | cggatcaagagctaccaactctttttccgaaggtaactggcttcagcaga | |
| | gcgcagataccaaatactgtccttctagtgtagccgtagttaggccacca | |
| | cttcaagaactctgtagcaccgcctacatacctcgctctgctaatcctgt | |
| | taccagtggctgctgccagtggcgataagtcgtgtcttaccgggttggac | |
| | tcaagacgatagttaccggataaggcgcagcggtcgggctgaacgggggg | |
| | ttcgtgcacacagcccagcttggagcgaacgacctacaccgaactgagat | |
| | acctacagcgtgagctatgagaaagcgccacgcttcccgaagggagaaag | |
| | gcggacaggtatccggtaagcggcagggtcggaacaggagagcgcacgag | |
| | ggagcttccagggggaaacgcctggtatctttatagtcctgtcgggtttc | |
| | gccacctctgacttgagcgtcgatttttgtgatgctcgtcaggggggcgg | |
| | agcctatggaaaaacgccagcaacgcggcctttttacggttcctggcaga | |
| | tcctagatgtggcgcaacgatgccggcgacaagcaggagcgcaccgactt | |
| | cttccgcatcaagtgttttggctctcaggccgaggcccacggcaagtatt | |
| | tgggcaaggggtcgctggtattcgtgcagggcaagattcggaataccaag | |
| | tacgagaaggacggccagacggtctacgggaccgacttcattgccgataa | |
| | ggtggattatctggacaccaaggcaccagggggtcaaatcaggaataagg | |
| | gcacattgccccggcgtgagtcggggcaatcccgcaaggagggtgaatga | |
| | atcggacgtttgaccggaaggcatacaggcaagaactgatcgacgcgggg | |
| | ttttccgccgaggatgccgaaaccatcgcaagccgcaccgtcatgcgtgc | |
| | gccccgcgaaaccttccagtccgtcggctcgatggtccagcaagctacgg | |
| | ccaagatcgagcgcgacagcgtgcaactggctccccctgccctgcccgcg | |
| | ccatcggccgccgtggagcgttcgcgtcgtcttgaacaggaggcggcagg | |
| | tttggcgaagtcgatgaccatcgacacgcgaggaactatgacgaccaaga | |
| | agcgaaaaaccgccggcgaggacctggcaaaacaggtcagcgaggccaag | |
| | caggccgcgttgctgaaacacacgaagcagcagatcaaggaaatgcagct | |
| | ttccttgttcgatattgcgccgtggccggacacgatgcgagcgatgccaa | |
| | acgacacggcccgctctgccctgttcaccacgcgcaacaagaaaatcccg | |
| | cgcgaggcgctgcaaaacaaggtcattttccacgtcaacaaggacgtgaa | |
| | gatcacctacaccggcgtcgagctgcgggccgacgatgacgaactggtgt | |
| | ggcagcaggtgttggagtacgcgaagcgcacccctatcggcgagccgatc | |
| | accttcacgttctacgagctttgccaggacctgggctggtcgatcaatgg | |
| | ccggtattacacgaaggccgaggaatgcctgtcgcgcctacaggcgacgg | |
| | cgatgggcttcacgtccgaccgcgttgggcacctggaatcggtgtcgctg | |
| | ctgcaccgcttccgcgtcctggaccgtggcaagaaaacgtcccgttgcca | |
| | ggtcctgatcgacgaggaaatcgtcgtgctgtttgctggcgaccactaca | |
| | cgaaattcatatgggagaagtaccgcaagctgtcgccgacggcccgacgg | |
| | atgttcgactatttcagctcgcaccgggagccgtacccgctcaagctgga | |
| | aaccttccgcctcatgtgcggatcggattccacccgcgtgaagaagtggc | |
| | gcgagcaggtcggcgaagcctgcgaagagttgcgaggcagcggcctggtg | |
| | gaacacgcctgggtcaatgatgacctggtgcattgcaaacgctagggcct | |
| | tgtggggtcagttccggctgggggttcagcagcccctgctcggatctgtt | |
| | ggaccggacagtagtcatggttgatgggctgcctgtatcgagtggtgatt | |
| | ttgtgccgagctgccggtcggggagctgttggctggctggtggcaggata | |
| | tattgtggtgtaaacaaattgacgcttagacaacttaataacacattgcg | |
| | gacgtttttaatgtactggggttgaacactct | |
| 100 | gtgccgaattcggatccagcgatctggattttagtactggattttggttt | RC4268 |
| | taggaattagaaattttattgatagaagtattttacaaatacaaatacat | M. Frank |
| | actaagggtttcttatatgctcaacacatgagcgaaaccctataggaacc | TA29b- |
| | ctaattcccttatctgggaactactcacacattattatggagaaactcga | P5U_ |
| | gcttgtcgatcgactctagctagagaagctcactttccaacaaccttagt | BARSTAR |
| | aatttctcccttaacataatgaacaaattcttcaagttgatcagctctag | Construct |
| | aagcaagtctatcttcttcttgtccaagataagcttgtctagcttcaaga | (RCGG30 |
| | ataactggttgatattgagctggaagtctttccatagcccaatcagcagc | 01) |
| | aacatcctttggagcaatctttccagtaacagcagaataccaaattctag | Plasmid |
| | aaagagtaagaacaacatttctttcatctccagcccaatctggtggagaa | comprising |
| | ttccaaagagtaagagtttcattaagagcttcaaaaagatcttgttctgg | construct |
| | aactggatcaaaaagttcttcagcagctggtccaacaagagcaacagaat | of SEQ ID |
| | gttctctagccttagtaagaagaatagcaagatcaatatcaatagtagct | NO: 44. |
| | ggttcaaaaattccagcaagaatatcatttctttgccattctccaaattg | This one |
| | aagttctctcttagctggatatctccatggaataatatcatcatgaacaa | is likely |
| | caatagtaacttcaacagctctaagaatttcagattctcctggagaagca | same |
| | gaagtttcaagaagatcattaataagagctcttctagtagtttcatcaag | plasmid as |
| | tctaacagtaacagtaacaagaagatcaatatcagaatgtggcttaagtc | SEQ ID |
| | ctccatcaacagcagatccataaagatgaacagcaagaagagtaggttca | NO: 61 |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagtttgtaata | |
| | ttatgatttacgtaaaaatctatctaatgattttatttaaactatagttt | |
| | tgaacttttccaactgaaatttcaacacagtaattatggttgcctgtaac | |
| | tcatattttgcaagccccacttcaattatgcagcctaacccaaaagtggg | |
| | gatatgtgatttcaaaggatatatgcatcaatcatttctccaaattagga | |
| | aagtaatttatataaaattaacttgactttaagttagaactgcattttac | |
| | tgaataaaagttataattattaataaataaatggttaagattttaacgtt | |
| | ataatatattcatacagtacaaattcaagtttttattcttgaaataaaat | |
| | ttttattttaggtatttcattittatgcagtcatgtgtcttttttttatt | |
| | tttgaaacatattttatggtcatttatatcaattcaaattagaattttct | |
| | tcctctctaactaattattcaacaacaaaaaaaaactattttttcaagtt | |
| | tttattttactcagtgttttaatcaatattaagcataatgagtatttata | |
| | aatcattctcaaaataatattttacatgttaaataatttttaagtatttt | |
| | taagagtgtgtttaattgtgttttaatttcttagtctattttttaaaaac | |
| | tattttatgaaacatttttcataaactgatgtttagagagtaaaagtgga | |
| | aaagtaagatgtgaaaatacaaggaagaacatgaataatagaaaattaaa | |
| | agtaaaaaacccaaacatcgatcatagagatgttatagttttttggtttt | |
| | gaaaaaactaaaaaaagagatagtagttttaaaaacagttttcgaaacta | |
| | tttttatcaaacaacataatttttttagcttttaaataaaaacagcttta | |
| | aaaaatgtccaggtttttttccaaataaattcaattccatattttaataa | |
| | gtaatggttaatccagtggtaggtatatattgtttaacacttgaaatttt | |
| | tttccattcaatttttttaatatttttcataacacttaatttatttatta | |
| | catatatcatatgttatattattatttttctctattctcttcctttctct | |
| | ctagttatttgtaaaaaaaaaaacatataatttatttccattgcataaat | |
| | tttatccatgtctatccatgcctatgtttttattctgccacatataccct | |
| | aagtgttagcaaatatgtgcacacattaacctaattaaactatactagaa | |
| | ctatatatcggacttgatagaaagtggagataagccaaggtagacaagaa | |
| | aaaggattgaacacatgatcatcctcatgtcacatgcagttgccatggat | |
| | ccaacaccaaagttatgatcagaaaacctctcaaggcaggtgactcacga | |
| | aatgaacaagccaatttgcttccaaaattcaccataaatagggacactac | |
| | tcaatgcaaaccatgcatacacaatcatatacagtgggattagtaaacaa | |
| | attaattaataaccaaaagttatttaaattgaagtcaatggctcaagtta | |
| | ttaatactttcgacggtgttgcagactaccttcaaacttatcacaagctg | |
| | ccagataactatatcacaaaatctgaagctcaggccctcggggggtagct | |
| | tcaaagggtaacctcgccgacgttgcacctggaaagagtattgggggcga | |
| | catctttagtaaccggtaagtttctgcttctacctttgatatatatataa | |
| | taattatcattaattagtagtaatataatatttcaaatatttttttcaaa | |
| | ataaaagaatgtagtatatagcaattgcttttctgtagtttataagtgtg | |
| | tatattttaatttataacttttctaatatatgaccaaaatttgttgatgt | |
| | gcagtgaaggtaaattgcctggaaaatctggacgtacatggcgcgaggcc | |
| | gatataaattacacaagcggattccgtaactctgacagaatactttattc | |
| | ttcagactggctgatatataaaacaacagatcattatcaaacatttacta | |
| | aaatacgttaggcttgtcaagcagatcgttcaaacatttggcaataaagt | |
| | ttcttaagattgaatcctgttgccggtcttgcgatgattatcatataatt | |
| | tctgttgaattacgttaagcatgtaataattaacatgtaatgcatgacgt | |
| | tatttatgagatgggtttttatgattagagtcccgcaattatacatttaa | |
| | tacgcgatagaaaacaaaatatagcgcgcaaactaggataaattatcgcg | |
| | cgcggtgtcatctatgttactagatcgacgctttacgaggatgcacatgt | |
| | gaccgagggacacgaagtgatccgtttaaactatcagtgtttgacaggat | |
| | atattggcgggtaaacctaagagaaaagagcgtttattagaataatcgga | |
| | tatttaaaagggcgtgaaaaggtttatccgttcgtccatttgtatgtgcc | |
| | agccgcctttgcgacgctcaccgggctggttgccctcgccgctgggctgg | |
| | cggccgtctatggccctgcaaacgcgccagaaacgccgtcgaagccgtgt | |
| | gcgagacaccgcggccgccggcgttgtggatacctcgcggaaaacttggc | |
| | cctcactgacagatgaggggcggacgttgacacttgaggggccgactcac | |
| | ccggcgcggcgttgacagatgaggggcaggctcgatttcggccggcgacg | |
| | tggagctggccagcctcgcaaatcggcgaaaacgcctgattttacgcgag | |
| | tttcccacagatgatgtggacaagcctggggataagtgccctgcggtatt | |
| | gacacttgaggggcgcgactactgacagatgaggggcgcgatccttgaca | |
| | cttgaggggcagagtgctgacagatgaggggcgcacctattgacatttga | |
| | ggggctgtccacaggcagaaaatccagcatttgcaagggtttccgcccgt | |
| | ttttcggccaccgctaacctgtcttttaacctgcttttaaaccaatattt | |
| | ataaaccttgtttttaaccagggctgcgccctgtgcgcgtgaccgcgcac | |
| | gccgaaggggggtgcccccccttctcgaaccctcccggcccgctaacggg | |
| | gcctcccatccccccaggggctgcgcccctcggccgcgaacggcctcacc | |
| | ccaaaaatggcagcgctggccaattcccgagtgcgcggaacccctatttg | |
| | tttatttttctaaatacattcaaatatgtatccgctcatgagacaataac | |
| | cctgataaatgcttcaataatattgaaaaaggaagagtatggctaaaatg | |
| | agaatatcaccggaattgaaaaaactgatcgaaaaataccgctgcgtaaa | |
| | agatacggaaggaatgtctcctgctaaggtatataagctggtgggagaaa | |
| | atgaaaacctatatttaaaaatgacggacagccggtataaagggaccacc | |
| | tatgatgtggaacgggaaaaggacatgatgctatggctggaaggaaagct | |
| | gcctgttccaaaggtcctgcactttgaacggcatgatggctggagcaatc | |
| | tgctcatgagtgaggccgatggcgtcctttgctcggaagagtatgaagat | |
| | gaacaaagccctgaaaagattatcgagctgtatgcggagtgcatcaggct | |
| | ctttcactccatcgacatatcggattgtccctatacgaatagcttagaca | |
| | gccgcttagccgaattggattacttactgaataacgatctggccgatgtg | |
| | gattgcgaaaactgggaagaggacactccatttaaagatccgcgcgagct | |
| | gtatgattttttaaagacggaaaagcccgaagaggaacttgtcttttccc | |
| | acggcgacctgggagacagcaacatctttgtgaaagatggcaaagtaagt | |
| | ggctttattgatcttgggagaagcggcagggggacaagtggtatgacatt | |
| | gccttctgcgtccggtcgctcagggaggatatcggggaagaacagtatgt | |
| | cgagctattttttgacttactggggatcaagcctgattgggagaaaataa | |
| | aatattatattttactggatgaattgttttagctgtcagaccaagtttac | |
| | tcatatatactttagattgatttaaaacttcatttttaatttaaaaggat | |
| | ctaggtgaagatcctttttgataatctcatgaccaaaatcccttaacgtg | |
| | agttttcgttccactgagcgtcagaccccgtagaaaagatcaaaggatct | |
| | tcttgagatcctttttttctgcgcgtaatctgctgcttgcaaacaaaaaa | |
| | accaccgctaccagcggtggtttgtttgccggatcaagagctaccaactc | |
| | tttttccgaaggtaactggcttcagcagagcgcagataccaaatactgtc | |
| | cttctagtgtagccgtagttaggccaccacttcaagaactctgtagcacc | |
| | gcctacatacctcgctctgctaatcctgttaccagtggctgctgccagtg | |
| | gcgataagtcgtgtcttaccgggttggactcaagacgatagttaccggat | |
| | aaggcgcagcggtcgggctgaacggggggttcgtgcacacagc | |
| 101 | ccagcttggagcgaacgacctacaccgaactgagatacctacagcgtgag | RC4267 |
| | ctatgagaaagcgccacgcttcccgaagggagaaaggcggacaggtatcc | M. Frank |
| | ggtaagcggcagggtcggaacaggagagcgcacgagggagcttccagggg | TA29a- |
| | gaaacgcctggtatctttatagtcctgtcgggtttcgccacctctgactt | P5U_BAR |
| | gagcgtcgatttttgtgatgctcgtcaggggggcggagcctatggaaaaa | STAR |
| | cgccagcaacgcggcctttttacggttcctggcagatcctagatgtggcg | Construct |
| | caacgatgccggcgacaagcaggagcgcaccgacttcttccgcatcaagt | (RCGG30 |
| | gttttggctctcaggccgaggcccacggcaagtatttgggcaaggggtcg | 00) |
| | ctggtattcgtgcagggcaagattcggaataccaagtacgagaaggacgg | Plasmid |
| | ccagacggtctacgggaccgacttcattgccgataaggtggattatctgg | comprising |
| | acaccaaggcaccaggcgggtcaaatcaggaataagggcacattgccccg | construct |
| | gcgtgagtcggggcaatcccgcaaggagggtgaatgaatcggacgtttga | of SEQ ID |
| | ccggaaggcatacaggcaagaactgatcgacgcggggttttccgccgagg | NO: 43. |
| | atgccgaaaccatcgcaagccgcaccgtcatgcgtgcgccccgcgaaacc | This one |
| | ttccagtccgtcggctcgatggtccagcaagctacggccaagatcgagcg | is likely |
| | cgacagcgtgcaactggctccccctgccctgcccgcgccatcggccgccg | same |
| | tggagcgttcgcgtcgtcttgaacaggaggcggcaggtttggcgaagtcg | plasmid as |
| | atgaccatcgacacgcgaggaactatgacgaccaagaagcgaaaaaccgc | SEQ ID |
| | cggcgaggacctggcaaaacaggtcagcgaggccaagcaggccgcgttgc | NO: 60 |
| | tgaaacacacgaagcagcagatcaaggaaatgcagctttccttgttcgat | |
| | attgcgccgtggccggacacgatgcgagcgatgccaaacgacacggcccg | |
| | ctctgccctgttcaccacgcgcaacaagaaaatcccgcgcgaggcgctgc | |
| | aaaacaaggtcattttccacgtcaacaaggacgtgaagatcacctacacc | |
| | ggcgtcgagctgcgggccgacgatgacgaactggtgtggcagcaggtgtt | |
| | ggagtacgcgaagcgcacccctatcggcgagccgatcaccttcacgttct | |
| | acgagctttgccaggacctgggctggtcgatcaatggccggtattacacg | |
| | aaggccgaggaatgcctgtcgcgcctacaggcgacggcgatgggcttcac | |
| | gtccgaccgcgttgggcacctggaatcggtgtcgctgctgcaccgcttcc | |
| | gcgtcctggaccgtggcaagaaaacgtcccgttgccaggtcctgatcgac | |
| | gaggaaatcgtcgtgctgtttgctggcgaccactacacgaaattcatatg | |
| | ggagaagtaccgcaagctgtcgccgacggcccgacggatgttcgactatt | |
| | tcagctcgcaccgggagccgtacccgctcaagctggaaaccttccgcctc | |
| | atgtgcggatcggattccacccgcgtgaagaagtggcgcgagcaggtcgg | |
| | cgaagcctgcgaagagttgcgaggcagcggcctggtggaacacgcctggg | |
| | tcaatgatgacctggtgcattgcaaacgctagggccttgtggggtcagtt | |
| | ccggctgggggttcagcagcccctgctcggatctgttggaccggacagta | |
| | gtcatggttgatgggctgcctgtatcgagtggtgattttgtgccgagctg | |
| | ccggtcggggagctgttggctggctggtggcaggatatattgtggtgtaa | |
| | acaaattgacgcttagacaacttaataacacattgcggacgtttttaatg | |
| | tactggggttgaacactctgtgccgaattcggatccagcgatctggattt | |
| | tagtactggattttggttttaggaattagaaattttattgatagaagtat | |
| | tttacaaatacaaatacatactaagggtttcttatatgctcaacacatga | |
| | gcgaaaccctataggaaccctaattcccttatctgggaactactcacaca | |
| | ttattatggagaaactcgagcttgtcgatcgactctagctagagaagctc | |
| | actttccaacaaccttagtaatttctcccttaacataatgaacaaattct | |
| | tcaagttgatcagctctagaagcaagtctatcttcttcttgtccaagata | |
| | agcttgtctagcttcaagaataactggttgatattgagctggaagtcttt | |
| | ccatagcccaatcagcagcaacatcctttggagcaatctttccagtaaca | |
| | gcagaataccaaattctagaaagagtaagaacaacatttctttcatctcc | |
| | agcccaatctggtggagaattccaaagagtaagagtttcattaagagctt | |
| | caaaaagatcttgttctggaactggatcaaaaagttcttcagcagctggt | |
| | ccaacaagagcaacagaatgttctctagccttagtaagaagaatagcaag | |
| | atcaatatcaatagtagctggttcaaaaattccagcaagaatatcatttc | |
| | tttgccattctccaaattgaagttctctcttagctggatatctccatgga | |
| | ataatatcatcatgaacaacaatagtaacttcaacagctctaagaatttc | |
| | agattctcctggagaagcagaagtttcaagaagatcattaataagagctc | |
| | ttctagtagtttcatcaagtctaacagtaacagtaacaagaagatcaata | |
| | tcagaatgtggcttaagtcctccatcaacagcagatccataaagatgaac | |
| | agcaagaagagtaggttcaagatgtctttcaataactccaacaacttcag | |
| | aaagttgagtagaaacttcagcaataacagcttctctcatgcaagcagta | |
| | gcaacagaagcagaaattctgaaagaacagaacttttgcatgaaaataga | |
| | atccttcttaagaacaagcatagaattagcagagttcttaagcttcttag | |
| | atccgaaaacaagaaaagaagatgacttaggaacttgtggcttatggaaa | |
| | ttagagttaggattaagagtttgaattccttgagccatattgttaatttg | |
| | agccattgtatcgataattgtaaatgtaattgtaatgttgtttgttgttt | |
| | gttgttgttggtaattgttgtaaaaatgagctcttatactcgagcgtgtc | |
| | ctctccaaatgaaatgaacttccttatatagaggaagggtcttgcgaagg | |
| | atagtgggattgtgcgtcatcccttacgtcagtggagatgtcacatcaat | |
| | ccacttgctttgtagacgtggttggaacctcttctttttccacgatgctc | |
| | ctcgtggggggggtccatctttgggaccactgtcggcagagagatcttga | |
| | atgatagcctttcctttatcgcaatgatggcatttgtaggagccaccttc | |
| | cttttctactgtcctttcgatgaagtgacagatagctgggcaatggaatc | |
| | cgaggaggtttcccgaaattatcctttgttgaaaagtctcaatagccctt | |
| | tgatcttctgagactgtatctttgacatttttggagtagaccagagtgtc | |
| | gtgctccaccatgttatcacatcaatccacttgctttgtagacgtggttg | |
| | gaacctcttctttttccacgatgctcctcgtggggggggtccatctttgg | |
| | gaccactgtcggcagagagatcttgaatgatagcctttcctttatcgcaa | |
| | tgatggcatttgtaggagccaccttccttttctactgtcctttcgatgaa | |
| | gtgacagatagctgggcaatggaatccgaggaggtttcccgaaattatcc | |
| | tttgttgaaaagtctcaatagccctttgatcttctgagactgtatctttg | |
| | acatttttggagtagaccagagtgtcgtgctccaccatgttgacctccgc | |
| | aagaattcaagcttagcgttcctaacacctggagaaccttttatgtactt | |
| | cacaaccctcaatgctgcttccaggtgtgactgtttgggttgctgcatga | |
| | attgactcagcacctgcactgcaaagctgatatctggccttgtgattgtc | |
| | aagtagagaagctttcccaccagtctctgataggatcccacatcctctaa | |
| | ctctgcatcatcagtctttcctaagtgcttgtcatattctacagtagtca | |
| | gcctttggttctgttccattggggttgacactggtctgcagcctcccaga | |
| | ccaacacctgatatcagttccagtgcatacttcctctggttcagtaggat | |
| | tcccttttctgatctcagcacctcaatgcctaagaagtattttagttctc | |
| | ccaaatctttcatcttgaaatgctgatgcagggttgcctttgcttctgaa | |
| | atcaaaacattgctgctgcctgttattaacagatcatccacataaatcag | |
| | gattatgacaaggtcagtcccctctcttttggtaaacaaggagtgatcat | |
| | aagcactttgcataaaaccagcctgcataaggacagtggtaagcttgatg | |
| | ttccactgccttgatgcttgctttaaaccatagagggattcaacagcctg | |
| | cacactttggtttccccttggctgtgaaaaccctgaggcagagacatata | |
| | aacttcttccatgaggtcaccttgtagaaaagcattgttgacatccatct | |
| | ggaaaaggaaccagcccttggaagcagcaacagatatgacagcttttaca | |
| | gtgaccattttggccactggagaaaaagtttcatggtagtcaaggccttc | |
| | ttgctgagtgtatcccttggccactagccttgccttaaacctgtcaactt | |
| | caccattagctttgtatttaattttgtacacccatttggaccctataggc | |
| | tgtttaccagggggtaaagggacaatctcccaggtgttattatcctcaag | |
| | agcctgtatctcaagggacatggcctccatccatttctcatcttgagctg | |
| | cttctttgaaggatttaggttcagtataagtggagaaagcactcaaataa | |
| | gcttgatagtgaacaggcaagtgatcataggaaacatagttggctatagg | |
| | gtatggaacatccctagagcctttgttcagtgtcacaaagtccttgagcc | |
| | agatgggaggacctgcattgcgtttaggtctagtgtgcaggtttgctgga | |
| | actagagagggatcagctacagcagtatggtgctcaaattcagcatttgc | |
| | taggtcaggctcagctgactcaactgactctgcaggagcatgcaggtggc | |
| | ctgaaggtgcagcatcagctgaagtgatatgattttgggtagaatgagtc | |
| | cccaaagtgatatcctcattggcctctacaatgtcaggaataagaactgg | |
| | agtatcatcatcatcagtatgaaagctggaaggaaaaatatcattataca | |
| | ctagctgcaaagctgtatcttcagagctaagtgaacctgctcgagtgaac | |
| | atatcaggttcatgggagatggactctttgaaagggaactgaaactctct | |
| | gaaaactacatccctgctcactatgatcaccttattatccaagtcataca | |
| | acctgtaacccttttgagtctcagaataaccaatgaagatggttcgcttg | |
| | gctctaggtgcaagtttgtcacctttgggcagtgttgctgcaaaagcaag | |
| | acacccaaacactctcaagtgatcaagcttggcatgtttctgatagagta | |
| | gttcatatggacatttgccttgtaagattggagtagggagtctattgatc | |
| | aggtatacaacagtcttgacacagtctccccaaaacctggtaggtacacc | |
| | actctgaaacttaagtgcccttgccatctcaaggatgtgtctgtgctttc | |
| | tctccacaacaccattctgttgtggtgtgtagggacagctactttgatga | |
| | acaatcccaagagaggccagcaactcattacaacttctccactagaattc | |
| | gagctcggaggtttccatagcctaaggccacgagtgcacgtgttcatagg | |
| | ctagttgtgttaaccttggggagcagtcgacaatttggattgcatcgagg | |
| | tcttgccaaattttactttcaagacagtgatttcgacggcacaataaact | |
| | tccaattagttatcttctcttttgttctgtattgtgtttttttctatttt | |
| | tctaacaatggaaatagtatataccggccaacagtgttgcactgttgtct | |
| | ggaccttgtcaaaggttcgacagtgcatagtttggactcgatgtggcata | |
| | tgggggttctgataagtgatgagattatgaaaataatttaattttagcta | |
| | ttaaaaatacaatacaactttcaaaagcgggaaacttgtttgattatgat | |
| | ctaggattctaggtctttttttttaagttcgagacagaaaaccaaaaaca | |
| | aaacagctaataagaagaagaaaaacaatacagcaatattcccacaaaaa | |
| | taaatttgttcttttgcgtactagccataggatttaattatatatatggg | |
| | ccagtccagaagcattgtcactgatagatcatagactagtaaaaaagaat | |
| | gaaccacttcagaacactaactatatatataaagatagattcatatctat | |
| | ttagatcgtgattattaatacaagaaaatatttgtatttcttaatttttt | |
| | gtttttaaatttatgtaaatatattgacatcttttttaacgaaaattcaa | |
| | attttaattcattatattacaagaaattaacacattttactagaccaact | |
| | ctcatgttacaacactatatatgttgaacattacaagaatgccactttat | |
| | ctcacccctttaattaggaaaagcaaaaatctatttgcatataattttgt | |
| | accaaaaaattatacaataaaaaagaaaaagagaaaagtaaaagataaga | |
| | cgactgatgtaataaaaagagagaaaaaataatactaaaaaataatataa | |
| | ataacataactcttaaaaaaaaaaggtaacactgttgagtatttttttta | |
| | ttacttttctctaaaaaggtatcatggtttcactaatgtccaggtttttt | |
| | ttttccaaataaactectttccggtggtaggtatcgtttcacacttgaat | |
| | acttttttgcaatatttttcgtaacactttatttctctccattacataat | |
| | tttacccttgcctatgtctttattctgccacatataccctaagagtgagc | |
| | aaatgtgtgcacgcatataacctgattggactatatatcggacttgatac | |
| | aaatgtgtgctcacatgaagagaaaaaggattgaacccatgatcatcatc | |
| | atgtcacatgcagatgccatgcattcaacaccaaagttatgatcagaaaa | |
| | cctctcaaggtaggtgactcaccaaatgaacaagccaattttttgcttcc | |
| | caaatttcaccataaatagggacactactccatccaaaccatacacgacc | |
| | acatacagtgggattagtaagtaaacaaattaattaataaccaaaagtta | |
| | taatttcaattgaatttagtcaatgaagaaggccgtcataaatggagaac | |
| | agatcaggagtatcagcgatctccatcaaactctgaagaaagaactcgct | |
| | ctgccagagtattacggtgaaaaccttgatgctctttgggactgcctcac | |
| | tggttgggtcgagtatccactcgtcctggaatggagacagttcgaacaat | |
| | ccaaacagctcaccgagaatggagctgaaagtgttttgcaagtctttagg | |
| | gaagcaaaagccgaaggctgtgacatcaccattatactctcttaggcttg | |
| | tcaagcagatcgttcaaacatttggcaataaagtttcttaagattgaatc | |
| | ctgttgccggtcttgcgatgattatcatataatttctgttgaattacgtt | |
| | aagcatgtaataattaacatgtaatgcatgacgttatttatgagatgggt | |
| | ttttatgattagagtcccgcaattatacatttaatacgcgatagaaaaca | |
| | aaatatagcgcgcaaactaggataaattatcgcgcgcggtgtcatctatg | |
| | ttactagatcgacgctttacgaattcccatggagcgtccctcggtcacat | |
| | gtgcatcctctagtggagaagttgtaatgagttgctggcctctcttggga | |
| | ttgttcatcaaagtagctgtccctacacaccacaacagaatggtgttgtg | |
| | gagagaaagcacagacacatccttgagatggcaagggcacttaagtttca | |
| | gagtggtgtacctaccaggttttggggagactgtgtcaagactgttgtat | |
| | acctgatcaatagactccctactccaatcttacaaggcaaatgtccatat | |
| | gaactactctatcagaaacatgccaagcttgatcacttgagagtgtttgg | |
| | gtgtcttgcttttgcagcaacactgcccaaaggtgacaaacttgcaccta | |
| | gagccaagcgaaccatcttcattggttattctgagactcaaaagggttac | |
| | aggttgtatgacttggataataaggtgatcatagtgagcagggatgtagt | |
| | tttcagagagtttcagttccctttcaaagagtccatctcccatgaacctg | |
| | atatgttcactcgagcaggttcacttagctctgaagatacagctttgcag | |
| | ctagtgtataatgatatttttccttccagctttcatactgatgatgatga | |
| | tactccagttcttattcctgacattgtagaggccaatgaggatatcactt | |
| | tggggactcattctacccaaaatcatatcacttcagctgatgctgcacct | |
| | tcaggccacctgcatgctcctgcagagtcagttgagtcagctgagcctga | |
| | cctagcaaatgctgaatttgagcaccatactgctgtagctgatccctctc | |
| | tagttccagcaaacctgcacactagacctaaacgcaatgcaggtcctccc | |
| | atctggctcaaggactttgtgacactgaacaaaggctctagggatgttcc | |
| | ataccctatagccaactatgtttcctatgatcacttgcctgttcactatc | |
| | aagcttatttgagtgctttctccacttatactgaacctaaatccttcaaa | |
| | gaagcagctcaagatgagaaatggatggaggccatgtcccttgagataca | |
| | ggctcttgaggataataacacctgggagattgtccctttaccccctggta | |
| | aacagcctatagggtccaaatgggtgtacaaaattaaatacaaagctaat | |
| | ggtgaagttgacaggtttaaggcaaggctagtggccaagggatacactca | |
| | gcaagaaggccttgactaccatgaaactttttctccagtggccaaaatgg | |
| | tcactgtaaaagctgtcatatctgttgctgcttccaagggctggttcctt | |
| | ttccagatggatgtcaacaatgcttttctacaaggtgacctcatggaaga | |
| | agtttatatgtctctgcctcagggttttcacagccaaggggaaaccaaag | |
| | tgtgcaggctgttgaatccctctatggtttaaagcaagcatcaaggcagt | |
| | ggaacatcaagcttaccactgtccttatgcaggctggttttatgcaaagt | |
| | gcttatgatcactccttgtttaccaaaagagaggggactgaccttgtcat | |
| | aatcctgatttatgtggatgatctgttaataacaggcagcagcaatgttt | |
| | tgatttcagaagcaaaggcaaccctgcatcagcatttcaagatgaaagat | |
| | ttgggagaactaaaatacttcttaggcattgaggtgctgagatcagaaaa | |
| | gggaatcctactgaaccagaggaagtatgcactggaactgatatcaggtg | |
| | ttggtctgggaggctgcagaccagtgtcaaccccaatggaacagaaccaa | |
| | aggctgactactgtagaatatgacaagcacttaggaaagactgatgatgc | |
| | agagttagaggatgtgggatcctatcagagactggtgggaaagcttctct | |
| | acttgacaatcacaaggccagatatcagctttgcagtgcaggtgctgagt | |
| | caattcatgcagcaacccaaacagtcacacctggaagcagcattgagggt | |
| | tgtgaagtacataaaaggttctccaggtgttaggaacgctaagcttgaat | |
| | tcttgcggaggacgtcgttgtggttggtgctttccttacattctgagcct | |
| | ctttccttctaatccactcatctgcatcttcttgtgtccttactaatacc | |
| | tcattggttccaaattccctccctttaagcaccagctcgtttctgttctt | |
| | ccacagcctcccaagtatccaagggactaaagcctccacattcttcagat | |
| | caggatattcttgtttaagatgttgaactctatggaggtttgtatgaact | |
| | gatgatctaggaccggataagttcccttcttcatagcgaacttattcaaa | |
| | gaatgttttgtgtatcattcttgttacattgttattaatgaaaaaatatt | |
| | attggtcattggactgaacacgagtgttaaatatggaccaggccccaaat | |
| | aagatccattgatatatgaattaaataacaagaataaatcgagtcaccaa | |
| | accacttgccttttttaacgagacttgttcaccaacttgatacaaaagtc | |
| | attatcctatgcaaatcaataatcatacaaaaatatccaataacactaaa | |
| | aaaattaaaagaaatggataatttcacaatatgttatacgataaagaagt | |
| | tacttttccaagaaattcactgattttataagcccacttgcattagataa | |
| | atggcaaaaaaaaacaaaaaggaaaagaaataaagcacgaagaattctag | |
| | aaaatacgaaatacgcttcaatgcagtgggacccacggttcaattattgc | |
| | caattttcagctccaccgtatatttaaaaaataaaacgataatgctaaaa | |
| | aaatataaatcgtaacgatcgttaaatctcaacggctggatcttatgacg | |
| | accgttaaggaaattgtggttgtcggacgaagtccagtaataaacggcgt | |
| | caaagtggttgcagccggcacacacgagtcgtgtttatcaactcaaagca | |
| | caaatacttttcctcaacctaaaaataaggcaattagccaaaaacaactt | |
| | tgcgtgtaaacaacgctcaatacacgtgtcattttattattagctattgc | |
| | ttcaccgccttagctttctcgtgacctagtcgtcctcgtcttttcttctt | |
| | cttcttctataaaacaatacccaaagagctcttcttcttcacaattcaga | |
| | tttcaatttctcaaaatcttaaaaactttctctcaattctctctaccgtg | |
| | atcaaggtaaatttctgtgttccttattctctcaaaatcttcgattttgt | |
| | tttcgttcgatcccaatttcgtatatgttctttggtttagattctgttaa | |
| | tcttagatcgtagacgattttctgggtttgatcgttagatatcatcttaa | |
| | ttctcgattagggtttcatagatatcatccgatttgttcaaataatttga | |
| | gttttgtcgaataattactcttcgatttgtgatttctatctagatctggt | |
| | gttagtttctagtttgtgcgatcgaatttgtcgattaatctgagtttttc | |
| | tgattaacagatgcagatcttaatggtcaagactaatcttttccttttct | |
| | tcatcttctcgcttctcctctcactttccagcgcggacatggtgtccaag | |
| | ggcgaagaggtgattaaagagttcatgaggttcaaagtgagaatggaggg | |
| | cagtatgaacggccatgagttcgagatcgaaggtgaaggcgaaggtagac | |
| | catacgagggcacccagacggcgaaactgaaagtgacgaagggcggccca | |
| | ctgcccttcgcctgggacattctgagtccacaatttatgtacggttccaa | |
| | ggcatacgttaagcaccccgccgacatccctgattacaaaaagctgtcgt | |
| | tcccggaggggtttaaatgggaaagagtgatgaattttgaggacggaggg | |
| | ctcgtcactgtgactcaagactcctcacttcaagacggcaccctaattta | |
| | caaggtgaagatgaggggaacaaatttcccccctgatggccctgttatgc | |
| | agaaaaaaacaatgggatgggaggcatccaccgagagactttacccgcgc | |
| | gacggagtgctcaaaggcgaaatccatcaggcactgaaactaaaggacgg | |
| | cggccactacctggttgagttcaaaactatatatatggccaagaaacccg | |
| | tccagttgccgggatattactatgtggacacaaaactggatataacgtcc | |
| | cacaacgaagattacaccatagtggaacagtatgagcgcagtgagggtcg | |
| | tcaccatctattcctcggacatggcacgggttccacgggctcgggttcca | |
| | gcggcacggcaagctcggaggacaacaacatggcggtgatcaaagagttc | |
| | atgcggtttaaagtccgtatggagggcagcatgaatgggcacgaatttga | |
| | aattgagggagaaggcgagggccgaccatatgaggggacacaaaccgcga | |
| | aactaaaagtcaccaaaggtgggccgcttccttttgcctgggacattctc | |
| | tctcctcagttcatgtatggctccaaggcctacgttaaacatccggctga | |
| | tatccccgattataagaaattgtctttcccggaggggtttaaatgggagc | |
| | gggttatgaatttcgaggatggtggactggttactgttacacaagattcg | |
| | agtctgcaggatggcacattgatatacaaagtcaaaatgaggggtacgaa | |
| | ttttcccccggacggcccggtgatgcagaaaaaaactatgggctgggagg | |
| | ccagcacggagaggctgtacccgagggacggggttttgaaaggggagata | |
| | caccaggcgcttaaactgaaggacggggggcactatctggtggaatttaa | |
| | gacgatatatatggcgaaaaagcccgtgcagctacctggctactactacg | |
| | tcgataccaaactcgatattacctctcataacgaggactacactatcgtg | |
| | gagcaatatgaaagatcagaaggacgtcaccacctttttctatacggaat | |
| | ggacgagctttacaaaaaggacgagttgtaagcttgtgtgtcttgtctta | |
| | tctggttcgtggtggtgagtttgttacaaaaaaatctattttccctagtt | |
| | gagatgggaattgaactatctgttgttatgtggattttattttctttttt | |
| | ctctttagaaccttatggttgtgtcaagaagtcttgtgtactttagtttt | |
| | atatctctgttttatctcttctattttctttaggatgcttgtgatgatgc | |
| | tgtttttttttgtccctaagcaaaaaaatatcatattatatttggtcctt | |
| | ggttcatttttttggtttttttttatcttcacatataaatattgtttgaa | |
| | tatcttcaatcttttatttgtatgagacaattatttaagtatcgggtgac | |
| | aatgcagctattatgtattgtcgattgttcgctggatccgaattcggcac | |
| | tcccagagaggatgcacatgtgaccgagggacacgaagtgatccgtttaa | |
| | actatcagtgtttgacaggatatattggcgggtaaacctaagagaaaaga | |
| | gcgtttattagaataatcggatatttaaaagggcgtgaaaaggtttatcc | |
| | gttcgtccatttgtatgtgccagccgcctttgcgacgctcaccgggctgg | |
| | ttgccctcgccgctgggctggcggccgtctatggccctgcaaacgcgcca | |
| | gaaacgccgtcgaagccgtgtgcgagacaccgcggccgccggcgttgtgg | |
| | atacctcgcggaaaacttggccctcactgacagatgaggggcggacgttg | |
| | acacttgaggggccgactcacccggcgcggcgttgacagatgaggggcag | |
| | gctcgatttcggccggcgacgtggagctggccagcctcgcaaatcggcga | |
| | aaacgcctgattttacgcgagtttcccacagatgatgtggacaagcctgg | |
| | ggataagtgccctgcggtattgacacttgaggggcgcgactactgacaga | |
| | tgaggggcgcgatccttgacacttgaggggcagagtgctgacagatgagg | |
| | ggcgcacctattgacatttgaggggctgtccacaggcagaaaatccagca | |
| | tttgcaagggtttccgcccgttttteggccaccgctaacctgtcttttaa | |
| | cctgcttttaaaccaatatttataaaccttgtttttaaccagggctgcgc | |
| | cctgtgcgcgtgaccgcgcacgccgaaggggggtgcccccccttctcgaa | |
| | ccctcccggcccgctaacgcgggcctcccatccccccaggggctgcgccc | |
| | ctcggccgcgaacggcctcaccccaaaaatggcagcgctggccaattccc | |
| | gagtgcgcggaacccctatttgtttatttttctaaatacattcaaatatg | |
| | tatccgctcatgagacaataaccctgataaatgcttcaataatattgaaa | |
| | aaggaagagtatggctaaaatgagaatatcaccggaattgaaaaaactga | |
| | tcgaaaaataccgctgcgtaaaagatacggaaggaatgtctcctgctaag | |
| | gtatataagctggtgggagaaaatgaaaacctatatttaaaaatgacgga | |
| | cagccggtataaagggaccacctatgatgtggaacgggaaaaggacatga | |
| | tgctatggctggaaggaaagctgcctgttccaaaggtcctgcactttgaa | |
| | cggcatgatggctggagcaatctgctcatgagtgaggccgatggcgtcct | |
| | ttgctcggaagagtatgaagatgaacaaagccctgaaaagattatcgagc | |
| | tgtatgcggagtgcatcaggctctttcactccatcgacatatcggattgt | |
| | ccctatacgaatagcttagacagccgcttagccgaattggattacttact | |
| | gaataacgatctggccgatgtggattgcgaaaactgggaagaggacactc | |
| | catttaaagatccgcgcgagctgtatgattttttaaagacggaaaagccc | |
| | gaagaggaacttgtcttttcccacggcgacctgggagacagcaacatctt | |
| | tgtgaaagatggcaaagtaagtggctttattgatcttgggagaagcggca | |
| | gggcggacaagtggtatgacattgccttctgcgtccggtcgctcagggag | |
| | gatatcggggaagaacagtatgtcgagctattttttgacttactggggat | |
| | caagcctgattgggagaaaataaaatattatattttactggatgaattgt | |
| | tttagctgtcagaccaagtttactcatatatactttagattgatttaaaa | |
| | cttcatttttaatttaaaaggatctaggtgaagatcctttttgataatct | |
| | catgaccaaaatcccttaacgtgagttttcgttccactgagcgtcagacc | |
| | ccgtagaaaagatcaaaggatcttcttgagatcctttttttctgcgcgta | |
| | atctgctgcttgcaaacaaaaaaaccaccgctaccagcggtggtttgttt | |
| | gccggatcaagagctaccaactctttttccgaaggtaactggcttcagca | |
| | gagcgcagataccaaatactgtccttctagtgtagccgtagttaggccac | |
| | cacttcaagaactctgtagcaccgcctacatacctcgctctgctaatcct | |
| | gttaccagtggctgctgccagtggcgataagtcgtgtcttaccgggttgg | |
| | actcaagacgatagttaccggataaggcgcagcggtcgggctgaacgggg | |
| | ggttcgtgcacacagcccagcttggagcgaacgacctacaccgaactgag | |
| | atacctacagcgtgagctatgagaaagcgccacgcttcccgaagggagaa | |
| | aggcggacaggtatccggtaagcggcagggtcggaacaggagagcgcacg | |
| | agggagcttccagggggaaacgcctggtatctttatagtcctgtcgggtt | |
| | tcgccacctctgacttgagcgtcgatttttgtgatgctcgtcaggggggc | |
| | ggagcctatggaaaaacgccagcaacgcggcctttttacggttcctggca | |
| | gatcctagatgtggcgcaacgatgccggcgacaagcaggagcgcaccgac | |
| | ttcttccgcatcaagtgttttggctctcaggccgaggcccacggcaagta | |
| | tttgggcaaggggtcgctggtattcgtgcagggcaagattcggaatacca | |
| | agtacgagaaggacggccagacggtctacgggaccgacttcattgccgat | |
| | aaggtggattatctggacaccaaggcaccaggcgggtcaaatcaggaata | |
| | agggcacattgccccggcgtgagtcggggcaatcccgcaaggagggtgaa | |
| | tgaatcggacgtttgaccggaaggcatacaggcaagaactgatcgacgcg | |
| | gggttttccgccgaggatgccgaaaccatcgcaagccgcaccgtcatgeg | |
| | tgcgccccgcgaaaccttccagtccgtcggctcgatggtccagcaagcta | |
| | cggccaagatcgagcgcgacagcgtgcaactggctccccctgccctgccc | |
| | gcgccatcggccgccgtggagcgttcgcgtcgtcttgaacaggaggcggc | |
| | aggtttggcgaagtcgatgaccatcgacacgcgaggaactatgacgacca | |
| | agaagcgaaaaaccgccggcgaggacctggcaaaacaggtcagcgaggcc | |
| | aagcaggccgcgttgctgaaacacacgaagcagcagatcaaggaaatgca | |
| | gctttccttgttcgatattgcgccgtggccggacacgatgcgagcgatgc | |
| | caaacgacacggcccgctctgccctgttcaccacgcgcaacaagaaaatc | |
| | ccgcgcgaggcgctgcaaaacaaggtcattttccacgtcaacaaggacgt | |
| | gaagatcacctacaccggcgtcgagctgcgggccgacgatgacgaactgg | |
| | tgtggcagcaggtgttggagtacgcgaagcgcacccctatcggcgagccg | |
| | atcaccttcacgttctacgagctttgccaggacctgggctggtcgatcaa | |
| | tggccggtattacacgaaggccgaggaatgcctgtcgcgcctacaggcga | |
| | cggcgatgggcttcacgtccgaccgcgttgggcacctggaatcggtgtcg | |
| | ctgctgcaccgcttccgcgtcctggaccgtggcaagaaaacgtcccgttg | |
| | ccaggtcctgatcgacgaggaaatcgtcgtgctgtttgctggcgaccact | |
| | acacgaaattcatatgggagaagtaccgcaagctgtcgccgacggcccga | |
| | cggatgttcgactatttcagctcgcaccgggagccgtacccgctcaagct | |
| | ggaaaccttccgcctcatgtgcggatcggattccacccgcgtgaagaagt | |
| | ggcgcgagcaggtcggcgaagcctgcgaagagttgcgaggcagcggcctg | |
| | gtggaacacgcctgggtcaatgatgacctggtgcattgcaaacgctaggg | |
| | ccttgtggggtcagttccggctgggggttcagcagcccctgctcggatct | |
| | gttggaccggacagtagtcatggttgatgggctgcctgtatcgagtggtg | |
| | attttgtgccgagctgccggtcggggagctgttggctggctggtggcagg | |
| | atatattgtggtgtaaacaaattgacgcttagacaacttaataacacatt | |
| | gcggacgtttttaatgtactggggttgaacactct | |
| 102 | gtgccgaattcggatccagcgatctggattttagtactggattttggttt | RC4266 |
| | taggaattagaaattttattgatagaagtattttacaaatacaaatacat | M. Frank |
| | actaagggtttcttatatgctcaacacatgagcgaaaccctataggaacc | TA29b- |
| | ctaattcccttatctgggaactactcacacattattatggagaaactcga | P5U_BAR |
| | gcttgtcgatcgactctagctagagaagctcactttccaacaaccttagt | NASE |
| | aatttctcccttaacataatgaacaaattcttcaagttgatcagctctag | Construct |
| | aagcaagtctatcttcttcttgtccaagataagcttgtctagcttcaaga | incorporated |
| | ataactggttgatattgagctggaagtctttccatagcccaatcagcagc | into |
| | aacatcctttggagcaatctttccagtaacagcagaataccaaattctag | plasmid |
| | aaagagtaagaacaacatttctttcatctccagcccaatctggtggagaa | vector. |
| | ttccaaagagtaagagtttcattaagagcttcaaaaagatcttgttctgg | (RCGG29 |
| | aactggatcaaaaagttcttcagcagctggtccaacaagagcaacagaat | 99) |
| | gttctctagccttagtaagaagaatagcaagatcaatatcaatagtagct | Plasmid |
| | ggttcaaaaattccagcaagaatatcatttctttgccattctccaaattg | comprising |
| | aagttctctcttagctggatatctccatggaataatatcatcatgaacaa | construct |
| | caatagtaacttcaacagctctaagaatttcagattctcctggagaagca | of SEQ ID |
| | gaagtttcaagaagatcattaataagagctcttctagtagtttcatcaag | NO: 42. |
| | tctaacagtaacagtaacaagaagatcaatatcagaatgtggcttaagtc | This one |
| | ctccatcaacagcagatccataaagatgaacagcaagaagagtaggttca | is likely |
| | agatgtctttcaataactccaacaacttcagaaagttgagtagaaacttc | same |
| | agcaataacagcttctctcatgcaagcagtagcaacagaagcagaaattc | plasmid as |
| | tgaaagaacagaacttttgcatgaaaatagaatccttcttaagaacaagc | SEQ ID |
| | atagaattagcagagttcttaagcttcttagatccgaaaacaagaaaaga | NO: 59 |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtottt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagtttgtaata | |
| | ttatgatttacgtaaaaatctatctaatgattttatttaaactatagttt | |
| | tgaacttttccaactgaaatttcaacacagtaattatggttgcctgtaac | |
| | tcatattttgcaagccccacttcaattatgcagcctaacccaaaagtggg | |
| | gatatgtgatttcaaaggatatatgcatcaatcatttctccaaattagga | |
| | aagtaatttatataaaattaacttgactttaagttagaactgcattttac | |
| | tgaataaaagttataattattaataaataaatggttaagattttaacgtt | |
| | ataatatattcatacagtacaaattcaagtttttattcttgaaataaaat | |
| | ttttattttaggtatttcatttttatgcagtcatgtgtcttttttttatt | |
| | tttgaaacatattttatggtcatttatatcaattcaaattagaattttct | |
| | tcctctctaactaattattcaacaacaaaaaaaaactattttttcaagtt | |
| | tttattttactcagtgttttaatcaatattaagcataatgagtatttata | |
| | aatcattctcaaaataatattttacatgttaaataatttttaagtatttt | |
| | taagagtgtgtttaattgtgttttaatttcttagtctattttttaaaaac | |
| | tattttatgaaacatttttcataaactgatgtttagagagtaaaagtgga | |
| | aaagtaagatgtgaaaatacaaggaagaacatgaataatagaaaattaaa | |
| | agtaaaaaacccaaacatcgatcatagagatgttatagttttttggtttt | |
| | gaaaaaactaaaaaaagagatagtagttttaaaaacagttttcgaaacta | |
| | tttttatcaaacaacataatttttttagcttttaaataaaaacagcttta | |
| | aaaaatgtccaggtttttttccaaataaattcaattccatattttaataa | |
| | gtaatggttaatccagtggtaggtatatattgtttaacacttgaaatttt | |
| | tttccattcaatttttttaatatttttcataacacttaatttatttatta | |
| | catatatcatatgttatattattatttttctctattctcttcctttctct | |
| | ctagttatttgtaaaaaaaaaaacatataatttatttccattgcataaat | |
| | tttatccatgtctatccatgcctatgtttttattctgccacatataccct | |
| | aagtgttagcaaatatgtgcacacattaacctaattaaactatactagaa | |
| | ctatatatcggacttgatagaaagtggagataagccaaggtagacaagaa | |
| | aaaggattgaacacatgatcatcctcatgtcacatgcagttgccatggat | |
| | ccaacaccaaagttatgatcagaaaacctctcaaggcaggtgactcacga | |
| | aatgaacaagccaatttgcttccaaaattcaccataaatagggacactac | |
| | tcaatgcaaaccatgcatacacaatcatatacagtgggattagtaaacaa | |
| | attaattaataaccaaaagttatttaaattgaagtcaatggctcaagtta | |
| | ttaatactttcgacggtgttgcagactaccttcaaacttatcacaagctg | |
| | ccagataactatatcacaaaatctgaagctcaggccctcggggggtagct | |
| | tcaaagggtaacctcgccgacgttgcacctggaaagagtattgggggcga | |
| | catctttagtaaccggtaagtttctgcttctacctttgatatatatataa | |
| | taattatcattaattagtagtaatataatatttcaaatatttttttcaaa | |
| | ataaaagaatgtagtatatagcaattgcttttctgtagtttataagtgtg | |
| | tatattttaatttataacttttctaatatatgaccaaaatttgttgatgt | |
| | gcagtgaaggtaaattgcctggaaaatctggacgtacatggcgcgaggcc | |
| | gatataaattacacaagcggattccgtaactctgacagaatactttattc | |
| | ttcagactggctgatatataaaacaacagatcattatcaaacatttacta | |
| | aaatacgttaggcttgtcaagcagatcgttcaaacatttggcaataaagt | |
| | ttcttaagattgaatcctgttgccggtcttgcgatgattatcatataatt | |
| | tctgttgaattacgttaagcatgtaataattaacatgtaatgcatgacgt | |
| | tatttatgagatgggtttttatgattagagtcccgcaattatacatttaa | |
| | tacgcgatagaaaacaaaatatagcgcgcaaactaggataaattatcgcg | |
| | cgcggtgtcatctatgttactagatcgacgctttacgaggatgcacatgt | |
| | gaccgagggacacgaagtgatccgtttaaactatcagtgtttgacaggat | |
| | atattgggggtaaacctaagagaaaagagcgtttattagaataatcggat | |
| | atttaaaagggcgtgaaaaggtttatccgttcgtccatttgtatgtgcca | |
| | gccgcctttgcgacgctcaccgggctggttgccctcgccgctgggctggc | |
| | ggccgtctatggccctgcaaacgcgccagaaacgccgtcgaagccgtgtg | |
| | cgagacaccgcggccgccggcgttgtggatacctcgcggaaaacttggcc | |
| | ctcactgacagatgaggggcggacgttgacacttgaggggccgactcacc | |
| | cggcgcggcgttgacagatgaggggcaggctcgatttcggccggcgacgt | |
| | ggagctggccagcctcgcaaatcggcgaaaacgcctgattttacgcgagt | |
| | ttcccacagatgatgtggacaagcctggggataagtgccctgcggtattg | |
| | acacttgaggggcgcgactactgacagatgaggggcgcgatccttgacac | |
| | ttgaggggcagagtgctgacagatgaggggcgcacctattgacatttgag | |
| | gggctgtccacaggcagaaaatccagcatttgcaagggtttccgcccgtt | |
| | tttcggccaccgctaacctgtcttttaacctgcttttaaaccaatattta | |
| | taaaccttgtttttaaccagggctgcgccctgtgcgcgtgaccgcgcacg | |
| | ccgaaggggggtgcccccccttctcgaaccctcccggcccgctaacgegg | |
| | gcctcccatccccccaggggctgcgcccctcggccgcgaacggcctcacc | |
| | ccaaaaatggcagcgctggccaattcccgagtgcgcggaacccctatttg | |
| | tttatttttctaaatacattcaaatatgtatccgctcatgagacaataac | |
| | cctgataaatgcttcaataatattgaaaaaggaagagtatggctaaaatg | |
| | agaatatcaccggaattgaaaaaactgatcgaaaaataccgctgcgtaaa | |
| | agatacggaaggaatgtctcctgctaaggtatataagctggtgggagaaa | |
| | atgaaaacctatatttaaaaatgacggacagccggtataaagggaccacc | |
| | tatgatgtggaacgggaaaaggacatgatgctatggctggaaggaaagct | |
| | gcctgttccaaaggtcctgcactttgaacggcatgatggctggagcaatc | |
| | tgctcatgagtgaggccgatggcgtcctttgctcggaagagtatgaagat | |
| | gaacaaagccctgaaaagattatcgagctgtatgcggagtgcatcaggct | |
| | ctttcactccatcgacatatcggattgtccctatacgaatagcttagaca | |
| | gccgcttagccgaattggattacttactgaataacgatctggccgatgtg | |
| | gattgcgaaaactgggaagaggacactccatttaaagatccgcgcgagct | |
| | gtatgattttttaaagacggaaaagcccgaagaggaacttgtcttttccc | |
| | acggcgacctgggagacagcaacatctttgtgaaagatggcaaagtaagt | |
| | ggctttattgatcttgggagaagcggcagggcggacaagtggtatgacat | |
| | tgccttctgcgtccggtcgctcagggaggatatcggggaagaacagtatg | |
| | tcgagctattttttgacttactggggatcaagcctgattgggagaaaata | |
| | aaatattatattttactggatgaattgttttagctgtcagaccaagttta | |
| | ctcatatatactttagattgatttaaaacttcatttttaatttaaaagga | |
| | tctaggtgaagatcctttttgataatctcatgaccaaaatcccttaacgt | |
| | gagttttcgttccactgagcgtcagaccccgtagaaaagatcaaaggatc | |
| | ttcttgagatcctttttttctgcgcgtaatctgctgcttgcaaacaaaaa | |
| | aaccaccgctaccagcggtggtttgtttgccggatcaagagctaccaact | |
| | ctttttccgaaggtaactggcttcagcagagcgcagataccaaatactgt | |
| | ccttctagtgtagccgtagttaggccaccacttcaagaactctgtagcac | |
| | cgcctacatacctcgctctgctaatcctgttaccagtggctgctgccagt | |
| | ggcgataagtcgtgtcttaccgggttggactcaagacgatagttaccgga | |
| | taaggcgcagcggtcgggctgaacggggggttcgtgcacacagcccagct | |
| | tggagcgaacgacctacaccgaactgagatacctacagcgtgagctatga | |
| | gaaagcgccacgcttcccgaagggagaaaggcggacaggtatccggtaag | |
| | cggcagggtcggaacaggagagcgcacgagggagcttccagggggaaacg | |
| | cctggtatctttatagtcctgtcgggtttcgccacctctgacttgagcgt | |
| | cgatttttgtgatgctcgtcaggggggcggagcctatggaaaaacgccag | |
| | caacgcggcctttttacggttcctggcagatcctagatgtggcgcaacga | |
| | tgccggcgacaagcaggagcgcaccgacttcttccgcatcaagtgttttg | |
| | gctctcaggccgaggcccacggcaagtatttgggcaaggggtcgctggta | |
| | ttcgtgcagggcaagattcggaataccaagtacgagaaggacggccagac | |
| | ggtctacgggaccgacttcattgccgataaggtggattatctggacacca | |
| | aggcaccaggcgggtcaaatcaggaataagggcacattgccccggcgtga | |
| | gtcggggcaatcccgcaaggagggtgaatgaatcggacgtttgaccggaa | |
| | ggcatacaggcaagaactgatcgacgcggggttttccgccgaggatgccg | |
| | aaaccatcgcaagccgcaccgtcatgcgtgcgccccgcgaaaccttccag | |
| | tccgtcggctcgatggtccagcaagctacggccaagatcgagcgcgacag | |
| | cgtgcaactggctccccctgccctgcccgcgccateggccgccgtggagc | |
| | gttcgcgtcgtcttgaacaggaggcggcaggtttggcgaagtcgatgacc | |
| | atcgacacgcgaggaactatgacgaccaagaagcgaaaaaccgccggcga | |
| | ggacctggcaaaacaggtcagcgaggccaagcaggccgcgttgctgaaac | |
| | acacgaagcagcagatcaaggaaatgcagctttccttgttcgatattgcg | |
| | ccgtggccggacacgatgcgagcgatgccaaacgacacggcccgctctgc | |
| | cctgttcaccacgcgcaacaagaaaatcccgcgcgaggcgctgcaaaaca | |
| | aggtcattttccacgtcaacaaggacgtgaagatcacctacaccggegtc | |
| | gagctgcgggccgacgatgacgaactggtgtggcagcaggtgttggagta | |
| | cgcgaagcgcacccctatcggcgagccgatcaccttcacgttctacgagc | |
| | tttgccaggacctgggctggtcgatcaatggccggtattacacgaaggcc | |
| | gaggaatgcctgtcgcgcctacaggcgacggcgatgggcttcacgtccga | |
| | ccgcgttgggcacctggaatcggtgtcgctgctgcaccgcttccgcgtcc | |
| | tggaccgtggcaagaaaacgtcccgttgccaggtcctgatcgacgaggaa | |
| | atcgtcgtgctgtttgctggcgaccactacacgaaattcatatgggagaa | |
| | gtaccgcaagctgtcgccgacggcccgacggatgttcgactatttcagct | |
| | cgcaccgggagccgtacccgctcaagctggaaaccttccgcctcatgtgc | |
| | ggatcggattccacccgcgtgaagaagtggcgcgagcaggtcggcgaagc | |
| | ctgcgaagagttgcgaggcagcggcctggtggaacacgcctgggtcaatg | |
| | atgacctggtgcattgcaaacgctagggccttgtggggtcagttccggct | |
| | gggggttcagcagcccctgctcggatctgttggaccggacagtagtcatg | |
| | gttgatgggctgcctgtatcgagtggtgattttgtgccgagctgccggtc | |
| | ggggagctgttggctggctggtggcaggatatattgtggtgtaaacaaat | |
| | tgacgcttagacaacttaataacacattgcggacgtttttaatgtactgg | |
| | ggttgaacactct | |
| 103 | gtgccgaattcggatccagcgatctggattttagtactggattttggttt | RC4265 |
| | taggaattagaaattttattgatagaagtattttacaaatacaaatacat | M. Frank |
| | actaagggtttcttatatgctcaacacatgagcgaaaccctataggaacc | W1pro- |
| | ctaattcccttatctgggaactactcacacattattatggagaaactcga | W1CDS- |
| | gcttgtcgatcgactctagctagagaagctcactttccaacaaccttagt | W1_3U |
| | aatttctcccttaacataatgaacaaattcttcaagttgatcagctctag | Construct |
| | aagcaagtctatcttcttcttgtccaagataagcttgtctagcttcaaga | (RCGG29 |
| | ataactggttgatattgagctggaagtctttccatagcccaatcagcagc | 98) |
| | aacatcctttggagcaatctttccagtaacagcagaataccaaattctag | Plasmid |
| | aaagagtaagaacaacatttctttcatctccagcccaatctggtggagaa | comprising |
| | ttccaaagagtaagagtttcattaagagcttcaaaaagatcttgttctgg | expression |
| | aactggatcaaaaagttcttcagcagctggtccaacaagagcaacagaat | construct |
| | gttctctagccttagtaagaagaatagcaagatcaatatcaatagtagct | of SEQ ID |
| | ggttcaaaaattccagcaagaatatcattttttgccattctccaaattga | NO: 73 |
| | agttctctcttagctggatatctccatggaataatatcatcatgaacaac | |
| | aatagtaacttcaacagctctaagaatttcagattctcctggagaagcag | |
| | aagtttcaagaagatcattaataagagctcttctagtagtttcatcaagt | |
| | ctaacagtaacagtaacaagaagatcaatatcagaatgtggcttaagtcc | |
| | tccatcaacagcagatccataaagatgaacagcaagaagagtaggttcaa | |
| | gatgtctttcaataactccaacaacttcagaaagttgagtagaaacttca | |
| | gcaataacagcttctctcatgcaagcagtagcaacagaagcagaaattct | |
| | gaaagaacagaacttttgcatgaaaatagaatccttcttaagaacaagca | |
| | tagaattagcagagttcttaagcttcttagatccgaaaacaagaaaagaa | |
| | gatgacttaggaacttgtggcttatggaaattagagttaggattaagagt | |
| | ttgaattccttgagccatattgttaatttgagccattgtatcgataattg | |
| | taaatgtaattgtaatgttgtttgttgtttgttgttgttggtaattgttg | |
| | taaaaatgagctcttatactcgagcgtgtcctctccaaatgaaatgaact | |
| | tccttatatagaggaagggtcttgcgaaggatagtgggattgtgcgtcat | |
| | cccttacgtcagtggagatgtcacatcaatccacttgctttgtagacgtg | |
| | gttggaacctcttctttttccacgatgctcctcgtggggggggtccatct | |
| | ttgggaccactgtcggcagagagatcttgaatgatagcctttcctttatc | |
| | gcaatgatggcatttgtaggagccaccttccttttctactgtcctttcga | |
| | tgaagtgacagatagctgggcaatggaatccgaggaggtttcccgaaatt | |
| | atcctttgttgaaaagtctcaatagccctttgatcttctgagactgtatc | |
| | tttgacatttttggagtagaccagagtgtcgtgctccaccatgttatcac | |
| | atcaatccacttgctttgtagacgtggttggaacctcttctttttccacg | |
| | atgctcctcgtggggggggtccatctttgggaccactgtcggcagagaga | |
| | tcttgaatgatagcctttcctttatcgcaatgatggcatttgtaggagcc | |
| | accttccttttctactgtcctttcgatgaagtgacagatagctgggcaat | |
| | ggaatccgaggaggtttcccgaaattatcctttgttgaaaagtctcaata | |
| | gccctttgatcttctgagactgtatctttgacatttttggagtagaccag | |
| | agtgtcgtgctccaccatgttgacctccgcaagaattcaagcttagcgtt | |
| | cctaacacctggagaaccttttatgtacttcacaaccctcaatgctgctt | |
| | ccaggtgtgactgtttgggttgctgcatgaattgactcagcacctgcact | |
| | gcaaagctgatatctggccttgtgattgtcaagtagagaagctttcccac | |
| | cagtctctgataggatcccacatcctctaactctgcatcatcagtctttc | |
| | ctaagtgcttgtcatattctacagtagtcagcctttggttctgttccatt | |
| | ggggttgacactggtctgcagcctcccagaccaacacctgatatcagttc | |
| | cagtgcatacttcctctggttcagtaggattcccttttctgatctcagca | |
| | cctcaatgcctaagaagtattttagttctcccaaatctttcatcttgaaa | |
| | tgctgatgcagggttgcctttgcttctgaaatcaaaacattgctgctgcc | |
| | tgttattaacagatcatccacataaatcaggattatgacaaggtcagtcc | |
| | cctctcttttggtaaacaaggagtgatcataagcactttgcataaaacca | |
| | gcctgcataaggacagtggtaagcttgatgttccactgccttgatgcttg | |
| | ctttaaaccatagagggattcaacagcctgcacactttggtttccccttg | |
| | gctgtgaaaaccctgaggcagagacatataaacttcttccatgaggtcac | |
| | cttgtagaaaagcattgttgacatccatctggaaaaggaaccagcccttg | |
| | gaagcagcaacagatatgacagcttttacagtgaccattttggccactgg | |
| | agaaaaagtttcatggtagtcaaggccttcttgctgagtgtatcccttgg | |
| | ccactagccttgccttaaacctgtcaacttcaccattagctttgtattta | |
| | attttgtacacccatttggaccctataggctgtttaccagggggtaaagg | |
| | gacaatctcccaggtgttattatcctcaagagcctgtatctcaagggaca | |
| | tggcctccatccatttctcatcttgagctgcttctttgaaggatttaggt | |
| | tcagtataagtggagaaagcactcaaataagcttgatagtgaacaggcaa | |
| | gtgatcataggaaacatagttggctatagggtatggaacatccctagagc | |
| | ctttgttcagtgtcacaaagtccttgagccagatgggaggacctgcattg | |
| | cgtttaggtctagtgtgcaggtttgctggaactagagagggatcagctac | |
| | agcagtatggtgctcaaattcagcatttgctaggtcaggctcagctgact | |
| | caactgactctgcaggagcatgcaggtggcctgaaggtgcagcatcagct | |
| | gaagtgatatgattttgggtagaatgagtccccaaagtgatatcctcatt | |
| | ggcctctacaatgtcaggaataagaactggagtatcatcatcatcagtat | |
| | gaaagctggaaggaaaaatatcattatacactagctgcaaagctgtatct | |
| | tcagagctaagtgaacctgctcgagtgaacatatcaggttcatgggagat | |
| | ggactctttgaaagggaactgaaactctctgaaaactacatccctgctca | |
| | ctatgatcaccttattatccaagtcatacaacctgtaacccttttgagtc | |
| | tcagaataaccaatgaagatggttcgcttggctctaggtgcaagtttgtc | |
| | acctttgggcagtgttgctgcaaaagcaagacacccaaacactctcaagt | |
| | gatcaagcttggcatgtttctgatagagtagttcatatggacatttgcct | |
| | tgtaagattggagtagggagtctattgatcaggtatacaacagtcttgac | |
| | acagtctccccaaaacctggtaggtacaccactctgaaacttaagtgccc | |
| | ttgccatctcaaggatgtgtctgtgctttctctccacaacaccattctgt | |
| | tgtggtgtgtagggacagctactttgatgaacaatcccaagagaggccag | |
| | caactcattacaacttctccactagaattcgagctcggagcacatgtcac | |
| | actctggtgcacatggtgttggtagaaagaccaaatgtcacgcccataac | |
| | tatcaatcgttgtcagtattgctatattccttggggatttacgactcatt | |
| | gacaaccagagtggttttaagacaactgtttttatctagctatggaagat | |
| | tctcacgatgctcaagaatttgaagtgatcaatggggtttgatgcaagag | |
| | gtaatgtcctgagttggagatttgagttattacgactcgtataggtcatt | |
| | gaggattgaggagatcacaccgatatgatgcccgaggaggaggaagtgca | |
| | tcatgagcttgtacgagatttagagccttcatcttaggaggaggattgat | |
| | taccttcctgtgtttataccttaggcgtaattcaatacttttagtcgggt | |
| | agtgatctgctctgaccctatatccttttttatatttgatgtatttgagg | |
| | ggtgagatcttcattgaaactgttttctttatgtaacagatgtgtatcga | |
| | taatttaattacttttacactttgggttgtatccaaatttcttctattgt | |
| | gacactatttatttgatttatttacatataaaggtttcatattataaatt | |
| | tgcgttcatttatttacatatttacttacgacgtgaaaacttgatgtttc | |
| | ttaagcaatgcatttattttattttatcttacttatacaaaaaaaatgat | |
| | gagatatttttttaacttaaaaatagaaattaaaagttaatgacacgtat | |
| | tccacaatatttattattcaaaccattttatgtaatatttaaaaaaaaag | |
| | tatatgagatgttacataaagtgaccataagttggaatacctcccactaa | |
| | caaaaagtaacaaataaattactaggattggccttaaaaaaatcgcgagt | |
| | tgttaattaatttactccttaatattataatagtcaattaattaaattgg | |
| | tccctaaaattaattgttttcttaaaaaaaataagtaaaacatgagaatc | |
| | caaatcaattttagttggtggttggtttgagtatttttttttcattttta | |
| | aagaaaaataaaataaaagtgaaaatacattttcttaaaatttagaaaat | |
| | atacataaaaacagatcaatagtaaagcaacgttttcaatcttttcttaa | |
| | gaagtaaaaatacggtgacactttgacattttttctcaatatatttttta | |
| | aatcttaaaaatttaaaataaaaaatatttttaaaaaataaaaataaaaa | |
| | taaaaaataaacactcaaactaaaataagtaaaagttttaattttcacaa | |
| | tgtggcgcattaagactctcgaaatgctatttccaaaaagaatggtagct | |
| | gactagatgcaatctctcgtaggcgttaggttggcaatggaaggtttggt | |
| | tgcacgcttgcacgccccatgtcagctgataacctactttcttagagtaa | |
| | cccactttccatattcacaaatgattaatggtcaatcttctaattcacgc | |
| | actactcaccccaaccatccattaggatattccgagtctaatggctccat | |
| | tattaaaaaaaaaaaatctttttatttaaattttaacttttcatttttat | |
| | atatttattcttaaacgaaaaatctaaactactatatttgattcatattc | |
| | tttatttattttttaaaatttccccattgtttaaattttttaaatcaaaa | |
| | tcaaaatcatttaagtttacttaattttaatattgtatgaaataattaaa | |
| | attaaaaaaaaatgtttttttaacaaagtaaaaagaaattataatggtga | |
| | tagaaccacgggaaagtatttgtgcaaaataattgaaattacaagtatta | |
| | ttttctattacaatttttttaattgtttctttgcttttttttttttttaa | |
| | cttctattttgcaagcatgtattttatatttaatgtgtgtggagggggac | |
| | ggagtacaaactcttttttgtctatattcagtaaaatgttggtggtgacg | |
| | tgccttgcgtgatgaactaccatttcaaacaccacacgacaccacacatc | |
| | cattttaaatataaccccatcatagatatcccgaatcatcaaattattac | |
| | ttcatagcaactagcaaattaattagcttcaccaatggactcattgttac | |
| | ttctaaaagaaattgccacttccattttgatcttcttgatcactcgcctc | |
| | tccattcaaacattcctcaaaagctategccagaaactcccaccggggcc | |
| | aaaagggtggccagttgtgggtgcactccctctcatgggaagcatgcctc | |
| | atgtcaccttagcaaagatggcaaaaaaatatggacctataatgtacctc | |
| | aaaatgggcactaacaacatggttgtggcctctactccagctgctgctcg | |
| | tgccttcctcaaaacccttgatcaaaacttttcaaaccggccctccaatg | |
| | ctggtgcaacccatttggcttatgatgcacgggatatggtgtttgctcat | |
| | tacggatcacggtggaagttgctaagaaaactaagtaacttgcacatgct | |
| | tggaggaaaggcacttgatgattgggcccaaattcgagatgaagagatgg | |
| | ggcacatgcttggtgcaatgtacgattgtaacaagagggatgaggctgtg | |
| | gtggtggcggagatgttgacatattcaatggccaacatgattggccaagt | |
| | tatattgagtcgtcgagtgtttgagacaaagggttcggagtctaacgagt | |
| | tcaaggacatggtggttgagctcatgaccgttgctggttacttcaacatt | |
| | ggtgacttcataccctttttggccaagttggacttgcaaggcatagagcg | |
| | tggcatgaagaagttgcacaagaagtttgatgcgttgttaacgagcatga | |
| | ttgaggagcatgttgcttctagtcacaagagaaagggcaagcccgatttc | |
| | ttagacatggtaatggctcatcatagtgagaactccgatggggaggaact | |
| | atcgctcaccaacatcaaggcactactcttgaacctattcaccgcaggca | |
| | ccgatacatcttcaagtataatagagtggtccttagccgagatgttgaag | |
| | aagcccagcataatgaagaaggctcatgaagaaatggaccaagtcatagg | |
| | aagggatcgccgcctcaaagaatctgacataccaaagcttccctacttcc | |
| | aagccatttgcaaagaaacctatagaaagcacccttcaacacccctaaac | |
| | ctgcctcgaatctcatctgaaccgtgccaagtgaatggttactacattcc | |
| | cgagaacactaggctgaatgtgaacatttgggccataggaagagatcctg | |
| | atgtgtggaacaatcctttggagtttatgcccgagaggtttttgagtggg | |
| | aagaatgccaaaattgacccacgtgggaatgattttgagcttattccatt | |
| | tggtgctgggaggaggatttgtgcagggactaggatggggattgtgttgg | |
| | ttcactacattttgggcactttggtgcattcgtttgattggaagctaccc | |
| | aatggggtgagggagttagacatggaggagtcctttgggcttgccttgca | |
| | aaaaaaggttccacttgctgctttggttacccctaggttgaacccaagtg | |
| | cttacatttcttaggcttaattggttgggttcgaatattcaccagctatg | |
| | ttctctagccttattttgttgtccaatgattttgtggctgtggctacata | |
| | aataagtaatgtttgggttgcacaacctatttgtatttgtaaggttctat | |
| | gttacttggaaatccgttaccccaccacctgcaaggcttagatttttatt | |
| | cttcatggatctctaatttagctgtcttgttttggtttaattatattttt | |
| | aattctcactcaggtagtcaagcagatcgttcaaacatttggcaataaag | |
| | tttcttaagattgaatcctgttgccggtcttgcgatgattatcatataat | |
| | ttctgttgaattacgttaagcatgtaataattaacatgtaatgcatgacg | |
| | ttatttatgagatgggtttttatgattagagtcccgcaattatacattta | |
| | atacgcgatagaaaacaaaatatagcgcgcaaactaggataaattatcgc | |
| | gcgcggtgtcatctatgttactagatcgacgctttacgaggatgcacatg | |
| | tgaccgagggacacgaagtgatccgtttaaactatcagtgtttgacagga | |
| | tatattggcgggtaaacctaagagaaaagagcgtttattagaataatcgg | |
| | atatttaaaagggcgtgaaaaggtttatccgttcgtccatttgtatgtgc | |
| | cagccgcctttgcgacgctcaccgggctggttgccctcgccgctgggctg | |
| | gcggccgtctatggccctgcaaacgcgccagaaacgccgtcgaagccgtg | |
| | tgcgagacaccgcggccgccggcgttgtggatacctegcggaaaacttgg | |
| | ccctcactgacagatgaggggcggacgttgacacttgaggggccgactca | |
| | cccggcgcggcgttgacagatgaggggcaggctcgatttcggccggcgac | |
| | gtggagctggccagcctcgcaaatcggcgaaaacgcctgattttacgcga | |
| | gtttcccacagatgatgtggacaagcctggggataagtgccctgcggtat | |
| | tgacacttgaggggcgcgactactgacagatgaggggcgcgatccttgac | |
| | acttgaggggcagagtgctgacagatgaggggcgcacctattgacatttg | |
| | aggggctgtccacaggcagaaaatccagcatttgcaagggtttccgcccg | |
| | tttttcggccaccgctaacctgtcttttaacctgcttttaaaccaatatt | |
| | tataaaccttgtttttaaccagggctgcgccctgtgcgcgtgaccgcgca | |
| | cgccgaaggggggtgcccccccttctcgaaccctcccggcccgctaacgc | |
| | gggcctcccatccccccaggggctgcgcccctcggccgcgaacggcctca | |
| | ccccaaaaatggcagcgctggccaattcccgagtgcgcggaacccctatt | |
| | tgtttatttttctaaatacattcaaatatgtatccgctcatgagacaata | |
| | accctgataaatgcttcaataatattgaaaaaggaagagtatggctaaaa | |
| | tgagaatatcaccggaattgaaaaaactgatcgaaaaataccgctgcgta | |
| | aaagatacggaaggaatgtctcctgctaaggtatataagctggtgggaga | |
| | aaatgaaaacctatatttaaaaatgacggacagccggtataaagggacca | |
| | cctatgatgtggaacgggaaaaggacatgatgctatggctggaaggaaag | |
| | ctgcctgttccaaaggtcctgcactttgaacggcatgatggctggagcaa | |
| | tctgctcatgagtgaggccgatggcgtcctttgctcggaagagtatgaag | |
| | atgaacaaagccctgaaaagattatcgagctgtatgcggagtgcatcagg | |
| | ctctttcactccatcgacatatcggattgtccctatacgaatagcttaga | |
| | cagccgcttagccgaattggattacttactgaataacgatctggccgatg | |
| | tggattgcgaaaactgggaagaggacactccatttaaagatccgcgcgag | |
| | ctgtatgattttttaaagacggaaaagcccgaagaggaacttgtcttttc | |
| | ccacggcgacctgggagacagcaacatctttgtgaaagatggcaaagtaa | |
| | gtggctttattgatcttgggagaagcggcagggcggacaagtggtatgac | |
| | attgccttctgcgtccggtcgctcagggaggatatcggggaagaacagta | |
| | tgtcgagctattttttgacttactggggatcaagcctgattgggagaaaa | |
| | taaaatattatattttactggatgaattgttttagctgtcagaccaagtt | |
| | tactcatatatactttagattgatttaaaacttcatttttaatttaaaag | |
| | gatctaggtgaagatcctttttgataatctcatgaccaaaatcccttaac | |
| | gtgagttttcgttccactgagcgtcagaccccgtagaaaagatcaaagga | |
| | tcttcttgagatcctttttttctgcgcgtaatctgctgcttgcaaacaaa | |
| | aaaaccaccgctaccagcggtggtttgtttgccggatcaagagctaccaa | |
| | ctctttttccgaaggtaactggcttcagcagagcgcagataccaaatact | |
| | gtccttctagtgtagccgtagttaggccaccacttcaagaactctgtagc | |
| | accgcctacatacctcgctctgctaatcctgttaccagtggctgctgcca | |
| | gtggcgataagtcgtgtcttaccgggttggactcaagacgatagttaccg | |
| | gataaggcgcagcggtcgggctgaacggggggttcgtgcacacagcccag | |
| | cttggagcgaacgacctacaccgaactgagatacctacagcgtgagctat | |
| | gagaaagcgccacgcttcccgaagggagaaaggcggacaggtatccggta | |
| | agcggcagggtcggaacaggagagcgcacgagggagcttccagggggaaa | |
| | cgcctggtatctttatagtcctgtcgggtttcgccacctctgacttgagc | |
| | gtcgatttttgtgatgctcgtcaggggggcggagcctatggaaaaacgcc | |
| | agcaacgcggcctttttacggttcctggcagatcctagatgtggcgcaac | |
| | gatgccggcgacaagcaggagcgcaccgacttcttccgcatcaagtgttt | |
| | tggctctcaggccgaggcccacggcaagtatttgggcaaggggtcgctgg | |
| | tattcgtgcagggcaagattcggaataccaagtacgagaaggacggccag | |
| | acggtctacgggaccgacttcattgccgataaggtggattatctggacac | |
| | caaggcaccaggcgggtcaaatcaggaataagggcacattgccccggcgt | |
| | gagtcggggcaatcccgcaaggagggtgaatgaatcggacgtttgaccgg | |
| | aaggcatacaggcaagaactgatcgacgcggggttttccgccgaggatgc | |
| | cgaaaccatcgcaagccgcaccgtcatgcgtgcgccccgcgaaaccttcc | |
| | agtccgtcggctcgatggtccagcaagctacggccaagatcgagcgcgac | |
| | agcgtgcaactggctccccctgccctgcccgcgccatcggccgccgtgga | |
| | gcgttcgcgtcgtcttgaacaggaggcggcaggtttggcgaagtcgatga | |
| | ccatcgacacgcgaggaactatgacgaccaagaagcgaaaaaccgccggc | |
| | gaggacctggcaaaacaggtcagcgaggccaagcaggccgcgttgctgaa | |
| | acacacgaagcagcagatcaaggaaatgcagctttccttgttcgatattg | |
| | cgccgtggccggacacgatgcgagcgatgccaaacgacacggcccgctct | |
| | gccctgttcaccacgcgcaacaagaaaatcccgcgcgaggcgctgcaaaa | |
| | caaggtcattttccacgtcaacaaggacgtgaagatcacctacaccggcg | |
| | tcgagctgcgggccgacgatgacgaactggtgtggcagcaggtgttggag | |
| | tacgcgaagcgcacccctatcggcgagccgatcaccttcacgttctacga | |
| | gctttgccaggacctgggctggtcgatcaatggccggtattacacgaagg | |
| | ccgaggaatgcctgtcgcgcctacaggcgacggcgatgggcttcacgtcc | |
| | gaccgcgttgggcacctggaatcggtgtcgctgctgcaccgcttccgcgt | |
| | cctggaccgtggcaagaaaacgtcccgttgccaggtcctgatcgacgagg | |
| | aaatcgtcgtgctgtttgctggcgaccactacacgaaattcatatgggag | |
| | aagtaccgcaagctgtcgccgacggcccgacggatgttcgactatttcag | |
| | ctcgcaccgggagccgtacccgctcaagctggaaaccttccgcctcatgt | |
| | gcggatcggattccacccgcgtgaagaagtggcgcgagcaggtcggcgaa | |
| | gcctgcgaagagttgcgaggcagcggcctggtggaacacgcctgggtcaa | |
| | tgatgacctggtgcattgcaaacgctagggccttgtggggtcagttccgg | |
| | ctgggggttcagcagcccctgctcggatctgttggaccggacagtagtca | |
| | tggttgatgggctgcctgtatcgagtggtgattttgtgccgagctgccgg | |
| | tcggggagctgttggctggctggtggcaggatatattgtggtgtaaacaa | |
| | attgacgcttagacaacttaataacacattgcggacgtttttaatgtact | |
| | ggggttgaacactct | |
| 104 | gtgccgaattcggatccagcgatctggattttagtactggattttggttt | RC4264 |
| | taggaattagaaattttattgatagaagtattttacaaatacaaatacat | M. Frank |
| | actaagggtttcttatatgctcaacacatgagegaaaccctataggaacc | SUC9-B |
| | ctaattcccttatctgggaactactcacacattattatggagaaactcga | gRNAs |
| | gcttgtcgatcgactctagctagagaagctcactttccaacaaccttagt | and |
| | aatttctcccttaacataatgaacaaattcttcaagttgatcagctctag | dCas9-TV |
| | aagcaagtctatcttcttcttgtccaagataagcttgtctagcttcaaga | Construct |
| | ataactggttgatattgagctggaagtctttccatagcccaatcagcagc | RCGG29 |
| | aacatcctttggagcaatctttccagtaacagcagaataccaaattctag | 97) |
| | aaagagtaagaacaacatttctttcatctccagcccaatctggtggagaa | |
| | ttccaaagagtaagagtttcattaagagcttcaaaaagatcttgttctgg | |
| | aactggatcaaaaagttcttcagcagctggtccaacaagagcaacagaat | |
| | gttctctagccttagtaagaagaatagcaagatcaatatcaatagtagct | |
| | ggttcaaaaattccagcaagaatatcatttctttgccattctccaaattg | |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagatatttgaa | |
| | tatacttaatgttgatgttaaatattatttcatccatcttaaaataagtg | |
| | tcattctatttattttatatagacaaaaaaaagttaataaattaataaaa | |
| | aaacagtaattttataaaactaaccttagaattaatatttcattaaaaaa | |
| | ctaaaattacacttattagaaatattagaaaaaaaatatcactactaaaa | |
| | atattattttttatgacgaacattctaaaatggttatatgaaatcatttt | |
| | agaatgtcatgcgatgacaattgtaaatacaagattgtttaacaaagacg | |
| | attttacaaaaattgtcttaaaaattattgttttatgacggtttgatgaa | |
| | aaccgtcttaaaatgattgtcattctaaaacgattttcgtaaaatcctct | |
| | taaaagttagtgcttttaatttttatgtctccctctcctctctcatttct | |
| | ctccctctcactctctactacttcttccgttccaaaataattgttgtatt | |
| | aggttattttacataggtgaagtaataaatagatgaaatataatgataat | |
| | tttagaaaattaatcttatatcatcatttaatacatttatatatagttca | |
| | tcattaataaataaaaaaataattaatgttacatttaaaaactaaaatga | |
| | taaaagtaattaatatacattaagaaactaaaatgacatatttttcttat | |
| | acgacaattataacacgatgagggagtaatgaatattagttaaaattagt | |
| | ttttgatcacctaatttataattttgattcaatttagtcctttagttatt | |
| | ttgggttcaatttaatcctctaatttttaaaattattttttttactttcc | |
| | attccatcacaagttccaaatgaatttgaatttaatcaacttatggtagt | |
| | atatatagaactttgaaagattattatctattgtaaaaaaattcaatctt | |
| | aagattttttttgcataacatgattcctaaactactcatcaggtgtctta | |
| | aagatcagttactagtgtaatggagctagctacttggctcacatttcagg | |
| | tattttacgtaaaagtccttacatgcactgtcgccctaacactttgttta | |
| | tgctaaccttgtcatcaaaatcaaaatgcagaaaagggaaaagaaagaaa | |
| | aaattcaaactggctttcccatccccataaagacttgctaaatataagga | |
| | gaagaagtgaagattatgatcagacatcacgtgggaacccttcttagtta | |
| | aaattgtaataattcaagagagagggtcaagagtcactggaaactctcag | |
| | gtttcaattttcaactgtacaaaataaaaattaaagagatgcaggggtca | |
| | gttaaaggaaataaatcttgtagatataaatatctatatatagattatgg | |
| | ttggataactttttatagaagaagaaaagaaaaacttgtcttataaattt | |
| | aaatcatcttacacatgcatgcacttgaattttatagaaactcttataag | |
| | ctaaatatataaattaattttaatttatgaaaatatatagtttgcttcat | |
| | tttactatttatttacttttgttataagtgtaaacaaggccatattcgaa | |
| | gtaaaaggccaaaaactcaaaatccaagaagcaaaatggtttctttattt | |
| | tttttatgctttaaaaatttgaagcttcggaattttgcgtgcaaactttt | |
| | atgtgcatgtccctctcattgaaattgaatcaaaccccacattaacaaat | |
| | taaagtcactttogaaaagaccctttgattatctccctctcctttttccc | |
| | atacatgaagaaatggatggtttatggagcatgaaaggtgaacctcaagt | |
| | ctctctctctctcttctctctcatgcttaaattctccaacacgagagtaa | |
| | attttagagtgagtggtaattgtggagggtctatttttctgtcataaata | |
| | cctgtctgctaggccaacccacttccatattttgcatcctttgtacctca | |
| | gttccaggttaacaccaaacaacacatatagtgcagattcataaggtata | |
| | gactttgttaatttgcaccctctcttaccctaaatcttgtgtctattgta | |
| | ttttatctctttccccatggattacaaagatgacgatgataaagactaca | |
| | aggacgacgacgataaaatggcccctaaaaagaagcgcaaggtgggtatt | |
| | catggggtgcctgcagctggaatggataagaagtactccatcggactggc | |
| | tatcggaactaactccgtgggatgggctgtgatcactgatgagtacaagg | |
| | tgccatccaagaagttcaaggtgctgggaaacactgatagacactccatc | |
| | aagaagaacctgatcggagctctgctgttcgattccggagagactgctga | |
| | ggctactagactgaagagaactgctagaagaagatacactagaagaaaga | |
| | acagaatctgctacctgcaagagatcttctccaacgagatggctaaggtg | |
| | gatgattccttcttccacagactggaggagtccttcctggtggaggagga | |
| | taagaagcacgagagacacccaatcttcggaaacatcgtggatgaggtgg | |
| | cttaccacgagaagtacccaactatctaccacctgagaaagaagctggtg | |
| | gattccactgataaggctgatctgagactgatctacctggctctggctca | |
| | catgatcaagttcagaggacacttcctgatcgagggagatctgaacccag | |
| | ataactccgatgtggataagctgttcatccaactggtgcaaacttacaac | |
| | caactgttcgaggagaacccaatcaacgcttccggagtggatgctaaggc | |
| | tatcctgtccgctagactgtccaagtccaggagactggagaacctgatcg | |
| | ctcaactgccaggagagaagaagaacggactgttcggaaacctgatcgct | |
| | ctgtccctgggactgactccaaacttcaagtccaacttcgatctggctga | |
| | ggatgctaagctgcaactgtccaaggatacttacgatgatgatctggata | |
| | acctgctggctcaaatcggagatcaatacgctgatctgttcctggctgct | |
| | aagaacctgtccgatgctatcctgctgtccgatatcctgagagtgaacac | |
| | tgagatcactaaggctccactgtccgcttccatgatcaagagatacgatg | |
| | agcaccaccaagatctgactctgctgaaggctctggtgagacaacaactg | |
| | ccagagaagtacaaggagatcttcttcgatcaatccaagaacggatacgc | |
| | tggatacatcgatggaggagcttcccaagaggagttctacaagttcatca | |
| | agccaatcctggagaagatggatggaactgaggagctgctggtgaagctg | |
| | aacagagaggatctgctgagaaagcaaagaactttcgataacggatccat | |
| | cccacaccaaatccacctgggagagctgcacgctatcctgaggagacaag | |
| | aggatttctacccattcctgaaggataacagagagaagatcgagaagatc | |
| | ctgactttcagaatcccatactacgtgggaccactggctagaggaaactc | |
| | cagattcgcttggatgactagaaagtccgaggagactatcactccatgga | |
| | acttcgaggaggtaagtttctgcttctacctttgatatatatataataat | |
| | tatcattaattagtagtaatataatatttcaaatattittttcaaaataa | |
| | aagaatgtagtatatagcaattgcttttctgtagtttataagtgtgtata | |
| | ttttaatttataacttttctaatatatgaccaaaatttgttgatgtgcag | |
| | gtggtggataagggagcttccgctcaatccttcatcgagagaatgactaa | |
| | cttcgataagaacctgccaaacgagaaggtgctgccaaagcactccctgc | |
| | tgtacgagtacttcactgtgtacaacgagctgactaaggtgaagtacgtg | |
| | actgagggaatgagaaagccagctttcctgtccggagagcaaaagaaggc | |
| | tatcgtggatctgctgttcaagactaacagaaaggtgactgtgaagcaac | |
| | tgaaggaggattacttcaagaagatcgagtgcttcgattccgtggagatc | |
| | tccggagtggaggatagattcaacgcttccctgggaacttaccacgatct | |
| | gctgaagatcatcaaggataaggatttcctggataacgaggagaacgagg | |
| | atatcctggaggatatcgtgctgactctgactctgttcgaggatagagag | |
| | atgatcgaggagagacttaagacttacgctcacctgttcgatgataaggt | |
| | gatgaagcaactgaagagaagaagatacactggatggggtagactgtcca | |
| | gaaagctgatcaacggaatcagagataagcaatccggaaagactatcctg | |
| | gatttcctgaagtccgatggattcgctaacagaaacttcatgcaactgat | |
| | ccacgatgattccctgactttcaaggaggatatccaaaaggctcaagtgt | |
| | ccggacaaggagattccctgcacgagcacatcgctaacctggctggatcc | |
| | ccagctatcaagaagggaatcctgcaaactgtgaaggtggtggatgagct | |
| | ggtgaaggtgatgggtagacacaagccagagaacatcgtgatcgagatgg | |
| | ctagagagaaccaaactactcaaaagggacaaaagaactccagagagaga | |
| | atgaagagaatcgaggagggaatcaaggagctgggatcccaaatcctgaa | |
| | ggagcacccagtggagaacactcaactgcaaaacgagaagctgtacctgt | |
| | actacctgcaaaacggaagagatatgtacgtggatcaagagctggatatc | |
| | aacagactgtccgattacgatgtggatgcaatcgtgccacaatccttcct | |
| | gaaggatgattccatcgataacaaggtgctgactagatccgataagaaca | |
| | gaggaaagtccgataacgtgccatccgaggaggtggtgaagaagatgaag | |
| | aactactggagacaactgctgaacgctaagctgatcactcaaagaaagtt | |
| | cgataacctgactaaggctgagagaggaggactgtccgagctggataagg | |
| | ctggattcatcaagagacaactggtggagactagacaaatcactaagcac | |
| | gtggctcaaatcctggattccagaatgaacactaagtacgatgagaacga | |
| | taagctgatcagagaggtgaaggtgatcactctgaagtccaagctggtgt | |
| | ccgatttcagaaaggatttccaattctacaaggtgagagagatcaacaac | |
| | taccaccacgctcacgatgcttacctgaacgctgtggtgggaactgctct | |
| | gatcaagaagtacccaaagctggagtccgagttcgtgtacggagattaca | |
| | aggtgtacgatgtgagaaagatgatcgctaagtccgagcaagagatcgga | |
| | aaggctactgctaagtacttcttctactccaacatcatgaacttcttcaa | |
| | gactgagatcactctggctaacggagagatcagaaaaagaccactgatcg | |
| | agactaacggagagactggagagatcgtgtgggataagggaagagatttc | |
| | gctactgtgagaaaggtgctgtccatgccacaagtgaacatcgtgaaaaa | |
| | gactgaggtgcaaactggaggattctccaaggagtccatcctgccaaaga | |
| | gaaactccgataagctgatcgctagaaagaaggattgggatccaaagaag | |
| | tacggaggattcgattccccaactgtggcttactccgtgctggtggtggc | |
| | taaggtggagaagggaaagtccaagaagctgaagtccgtgaaggagctgc | |
| | tgggaatcactatcatggagagatcctccttcgagaagaacccaatcgat | |
| | ttcctggaggctaagggatacaaggaggtgaagaaggatctgatcatcaa | |
| | gctgccaaagtactccctgttcgagctggagaacggaagaaagagaatgc | |
| | tggcttccgctggagagctgcaaaagggaaacgagctggctctgccatcc | |
| | aagtacgtgaacttcctgtacctggcttcccactacgagaagctgaaggg | |
| | atccccagaggataacgagcaaaagcaactgttcgtggagcaacacaagc | |
| | actacctggatgagatcatcgagcaaatctccgagttctccaagagagtg | |
| | atcctggctgatgctaacctggataaggtgctgtccgcttacaacaagca | |
| | cagagataagccaatcagagagcaagctgagaacatcatccacctgttca | |
| | ctctgactaacctgggagctccagctgctttcaagtacttcgatactact | |
| | atcgatagaaagagatacacttccactaaggaggtgctggatgctactct | |
| | gatccaccaatccatcactggactgtacgagactagaatcgatctgtccc | |
| | aactgggaggagatagttcgaagaggcctgccgccacaaagaaagccggc | |
| | caggcaaagaaaaaaaaacccaggggagggagtggtggactcctcgaccc | |
| | tggcactccaatggacgccgatctcgtagcatcatctaccgtcgtgtggg | |
| | agcaggacgccgacccttttgctggcacagctgatgatttcccagctttt | |
| | aatgaggaagaattggcttggctgatggagcttctcccacaaggtggtag | |
| | cggcgggctcctcgatcccggtactcccatggacgcagatcttgttgcta | |
| | gctccactgttgtatgggagcaggacgccgatcccttcgctggaaccgcc | |
| | gatgatttcccagcattcaacgaggaagaacttgcatggctcatggagct | |
| | cttgccccaagctcgcggtggatctgggggtttgttggatcccggaaccc | |
| | caatggacgcagacctcgtggcttctagtaccgtagtatgggaacaggac | |
| | gctgaccctttcgccggcaccgcagatgacttcccagcattcaacgagga | |
| | ggaactggcctggctgatggaacttctgcctcagggtggatccggaggcc | |
| | tcttggaccccggtacccctatggacgctgacctggtcgcatcttctacc | |
| | gtggtttgggagcaagatgccgacccattcgcaggaacagctgatgactt | |
| | ccccgcattcaatgaagaagagctcgcatggctgatggaattgcttccac | |
| | aagccagaggcggctctggagggcttcttgatcccggaacacccatggat | |
| | gccgacctcgtggcctctagcaccgtggtatgggaacaagacgcagatcc | |
| | attcgcagggaccgcagacgatttccccgcttttaacgaggaggagcttg | |
| | cttggctcatggagctgctgcctcagggtggatccgggggacttctcgac | |
| | ccaggcacacccatggatgccgatctcgtcgccagttctaccgttgtctg | |
| | ggagcaagacgcagaccctttcgccggaactgcagacgacttccctgcct | |
| | ttaatgaagaagaactggcttggctcatggaactgctcccccaggctaga | |
| | gggggtagcggcgggggcggcagcggaggtgatgcacttgacgacttcga | |
| | tcttgatatgcttggcagcgacgcacttgatgacttcgatttggatatgc | |
| | tggggtccgatgctcttgacgattttgatttggacatgttgggttcagac | |
| | gcactcgatgatttcgatttggacatgctggcacgtggaagcgatgccct | |
| | tgatgactttgacctggatatgctcgggtccgatgctttggacgacttcg | |
| | acctcgacatgttgggaagtgatgctcttgatgatttcgatcttgatatg | |
| | cttgggtccgacgctctcgatgattttgacctcgatatgctctgagcttg | |
| | tcaagcagatcgttcaaacatttggcaataaagtttcttaagattgaatc | |
| | ctgttgccggtcttgcgatgattatcatataatttctgttgaattacgtt | |
| | aagcatgtaataattaacatgtaatgcatgacgttatttatgagatgggt | |
| | ttttatgattagagtcccgcaattatacatttaatacgcgatagaaaaaa | |
| | aatatagcgcgcaaactaggataaattatcgcgcgcggtgtcatctatgt | |
| | tactagatcgacgctttacgaattcccatggggaggacgtcgttgtggtt | |
| | ggtgctttccttacattctgagcctctttccttctaatccactcatctgc | |
| | atcttcttgtgtccttactaatacctcattggttccaaattccctccctt | |
| | taagcaccagctcgtttctgttcttccacagcctcccaagtatccaaggg | |
| | actaaagcctccacattcttcagatcaggatattcttgtttaagatgttg | |
| | aactctatggaggtttgtatgaactgatgatctaggaccggataagttcc | |
| | cttcttcatagcgaacttattcaaagaatgttttgtgtatcattcttgtt | |
| | acattgttattaatgaaaaaatattattggtcattggactgaacacgagt | |
| | gttaaatatggaccaggccccaaataagatccattgatatatgaattaaa | |
| | taacaagaataaatcgagtcaccaaaccacttgccttttttaacgagact | |
| | tgttcaccaacttgatacaaaagtcattatcctatgcaaatcaataatca | |
| | tacaaaaatatccaataacactaaaaaaattaaaagaaatggataatttc | |
| | acaatatgttatacgataaagaagttacttttccaagaaattcactgatt | |
| | ttataagcccacttgcattagataaatggcaaaaaaaaacaaaaaggaaa | |
| | agaaataaagcacgaagaattctagaaaatacgaaatacgcttcaatgca | |
| | gtgggacccacggttcaattattgccaattttcagctccaccgtatattt | |
| | aaaaaataaaacgataatgctaaaaaaatataaatcgtaacgatcgttaa | |
| | atctcaacggctggatcttatgacgaccgttaaggaaattgtggttgtcg | |
| | gacgaagtccagtaataaacggcgtcaaagtggttgcagccggcacacac | |
| | gagtcgtgtttatcaactcaaagcacaaatacttttcctcaacctaaaaa | |
| | taaggcaattagccaaaaacaactttgcgtgtaaacaacgctcaatacac | |
| | gtgtcattttattattagctattgcttcaccgccttagctttctcgtgac | |
| | ctagtcgtcctcgtcttttcttcttcttcttctataaaacaatacccaaa | |
| | gagctcttcttcttcacaattcagatttcaatttctcaaaatcttaaaaa | |
| | ctttctctcaattctctctaccgtgatcaaggtaaatttctgtgttcctt | |
| | attctctcaaaatcttcgattttgttttcgttcgatcccaatttcgtata | |
| | tgttctttggtttagattctgttaatcttagatcgtagacgattttctgg | |
| | gtttgatcgttagatatcatcttaattctcgattagggtttcatagatat | |
| | catccgatttgttcaaataatttgagttttgtcgaataattactcttcga | |
| | tttgtgatttctatctagatctggtgttagtttctagtttgtgcgatcga | |
| | atttgtcgattaatctgagtttttctgattaacagatgcagatcttaatg | |
| | acaaagcaccagtggtctagtggtagaatagtaccctgccacggtacaga | |
| | cccgggttcgattcccggctggtgcatacgttgcatgtgttctttagttt | |
| | tagagctagaaatagcaagttaaaataaggctagtccgttatcaacttga | |
| | aaaagtggcaccgagtcggtgcaacaaagcaccagtggtctagtggtaga | |
| | atagtaccctgccacggtacagacccgggttcgattcccggctggtgcag | |
| | gagtgtgtgtaaatgtgaggttttagagctagaaatagcaagttaaaata | |
| | aggctagtccgttatcaacttgaaaaagtggcaccgagtcggtgcaacaa | |
| | agcaccagtggtctagtggtagaatagtaccctgccacggtacagacccg | |
| | ggttcgattcccggctggtgcacctaattctaaccgatattggttttaga | |
| | gctagaaatagcaagttaaaataaggctagtccgttatcaacttgaaaaa | |
| | gtggcaccgagtcggtgccaacaaagcaccagtggtctagtggtagaata | |
| | gtaccctgccacggtacagacccgggttcgattcccggctggtgcagctt | |
| | ctctagctagagtcgatcgacaagctcgagtttctccataataatgtgtg | |
| | agtagttcccagataagggaattagggttcctatagggtttcgctcatgt | |
| | gttgagcatataagaaacccttagtatgtatttgtatttgtaaaatactt | |
| | ctatcaataaaatttctaattcctaaaaccaaaatccagtactaaaatcc | |
| | agatcgctcagagaggatgcacatgtgaccgagggacacgaagtgatccg | |
| | tttaaactatcagtgtttgacaggatatattggcgggtaaacctaagaga | |
| | aaagagcgtttattagaataatcggatatttaaaagggcgtgaaaaggtt | |
| | tatccgttcgtccatttgtatgtgccagccgcctttgcgacgctcaccgg | |
| | gctggttgccctcgccgctgggctggcggccgtctatggccctgcaaacg | |
| | cgccagaaacgccgtcgaagccgtgtgcgagacaccgcggccgccggcgt | |
| | tgtggatacctcgcggaaaacttggccctcactgacagatgaggggcgga | |
| | cgttgacacttgaggggccgactcacccggcgcggcgttgacagatgagg | |
| | ggcaggctcgatttcggccggcgacgtggagctggccagcctcgcaaatc | |
| | ggcgaaaacgcctgattttacgcgagtttcccacagatgatgtggacaag | |
| | cctggggataagtgccctgcggtattgacacttgaggggcgcgactactg | |
| | acagatgaggggcgcgatccttgacacttgaggggcagagtgctgacaga | |
| | tgaggggcgcacctattgacatttgaggggctgtccacaggcagaaaatc | |
| | cagcatttgcaagggtttccgcccgtttttcggccaccgctaacctgtct | |
| | tttaacctgcttttaaaccaatatttataaaccttgtttttaaccagggc | |
| | tgcgccctgtgcgcgtgaccgcgcacgccgaaggggggtgcccccccttc | |
| | tcgaaccctcccggcccgctaacgcgggcctcccatccccccaggggctg | |
| | cgcccctcggccgcgaacggcctcaccccaaaaatggcagcgctggccaa | |
| | ttcccgagtgcgcggaacccctatttgtttatttttctaaatacattcaa | |
| | atatgtatccgctcatgagacaataaccctgataaatgcttcaataatat | |
| | tgaaaaaggaagagtatggctaaaatgagaatatcaccggaattgaaaaa | |
| | actgatcgaaaaataccgctgcgtaaaagatacggaaggaatgtctcctg | |
| | ctaaggtatataagctggtgggagaaaatgaaaacctatatttaaaaatg | |
| | acggacagccggtataaagggaccacctatgatgtggaacgggaaaagga | |
| | catgatgctatggctggaaggaaagctgcctgttccaaaggtcctgcact | |
| | ttgaacggcatgatggctggagcaatctgctcatgagtgaggccgatggc | |
| | gtcctttgctcggaagagtatgaagatgaacaaagccctgaaaagattat | |
| | cgagctgtatgcggagtgcatcaggctctttcactccatcgacatatcgg | |
| | attgtccctatacgaatagcttagacagccgcttagccgaattggattac | |
| | ttactgaataacgatctggccgatgtggattgcgaaaactgggaagagga | |
| | cactccatttaaagatccgcgcgagctgtatgattttttaaagacggaaa | |
| | agcccgaagaggaacttgtcttttcccacggcgacctgggagacagcaac | |
| | atctttgtgaaagatggcaaagtaagtggctttattgatcttgggagaag | |
| | cggcagggcggacaagtggtatgacattgccttctgcgtccggtcgctca | |
| | gggaggatatcggggaagaacagtatgtcgagctattttttgacttactg | |
| | gggatcaagcctgattgggagaaaataaaatattatattttactggatga | |
| | attgttttagctgtcagaccaagtttactcatatatactttagattgatt | |
| | taaaacttcatttttaatttaaaaggatctaggtgaagatcctttttgat | |
| | aatctcatgaccaaaatcccttaacgtgagttttcgttccactgagcgtc | |
| | agaccccgtagaaaagatcaaaggatcttcttgagatcctttttttctgc | |
| | gcgtaatctgctgcttgcaaacaaaaaaaccaccgctaccagcggtggtt | |
| | tgtttgccggatcaagagctaccaactctttttccgaaggtaactggctt | |
| | cagcagagcgcagataccaaatactgtccttctagtgtagccgtagttag | |
| | gccaccacttcaagaactctgtagcaccgcctacatacctcgctctgcta | |
| | atcctgttaccagtggctgctgccagtggcgataagtcgtgtcttaccgg | |
| | gttggactcaagacgatagttaccggataaggcgcagcggtcgggctgaa | |
| | cggggggttcgtgcacacagcccagcttggagcgaacgacctacaccgaa | |
| | ctgagatacctacagcgtgagctatgagaaagcgccacgcttcccgaagg | |
| | gagaaaggcggacaggtatccggtaagcggcagggtcggaacaggagagc | |
| | gcacgagggagcttccagggggaaacgcctggtatctttatagtcctgtc | |
| | gggtttcgccacctctgacttgagcgtcgatttttgtgatgctcgtcagg | |
| | ggggcggagcctatggaaaaacgccagcaacgcggcctttttacggttcc | |
| | tggcagatcctagatgtggcgcaacgatgccggcgacaagcaggagcgca | |
| | ccgacttcttccgcatcaagtgttttggctctcaggccgaggcccacggc | |
| | aagtatttgggcaaggggtcgctggtattcgtgcagggcaagattcggaa | |
| | taccaagtacgagaaggacggccagacggtctacgggaccgacttcattg | |
| | ccgataaggtggattatctggacaccaaggcaccagggggtcaaatcagg | |
| | aataagggcacattgccccggcgtgagtcggggcaatcccgcaaggaggg | |
| | tgaatgaatcggacgtttgaccggaaggcatacaggcaagaactgatcga | |
| | cgcggggttttccgccgaggatgccgaaaccatcgcaagccgcaccgtca | |
| | tgcgtgcgccccgcgaaaccttccagtccgtcggctcgatggtccagcaa | |
| | gctacggccaagatcgagcgcgacagcgtgcaactggctccccctgccct | |
| | gcccgcgccatcggccgccgtggagcgttcgcgtcgtcttgaacaggagg | |
| | cggcaggtttggcgaagtcgatgaccatcgacacgcgaggaactatgacg | |
| | accaagaagcgaaaaaccgccggcgaggacctggcaaaacaggtcagcga | |
| | ggccaagcaggccgcgttgctgaaacacacgaagcagcagatcaaggaaa | |
| | tgcagctttccttgttcgatattgcgccgtggccggacacgatgcgagcg | |
| | atgccaaacgacacggcccgctctgccctgttcaccacgcgcaacaagaa | |
| | aatcccgcgcgaggcgctgcaaaacaaggtcattttccacgtcaacaagg | |
| | acgtgaagatcacctacaccggcgtcgagctgcgggccgacgatgacgaa | |
| | ctggtgtggcagcaggtgttggagtacgcgaagcgcacccctatcggcga | |
| | gccgatcaccttcacgttctacgagctttgccaggacctgggctggtcga | |
| | tcaatggccggtattacacgaaggccgaggaatgcctgtcgcgcctacag | |
| | gcgacggcgatgggcttcacgtccgaccgcgttgggcacctggaatcggt | |
| | gtcgctgctgcaccgcttccgcgtcctggaccgtggcaagaaaacgtccc | |
| | gttgccaggtcctgatcgacgaggaaatcgtcgtgctgtttgctggcgac | |
| | cactacacgaaattcatatgggagaagtaccgcaagctgtcgccgacggc | |
| | ccgacggatgttcgactatttcagctcgcaccgggagccgtacccgctca | |
| | agctggaaaccttccgcctcatgtgcggatcggattccacccgcgtgaag | |
| | aagtggcgcgagcaggtcggcgaagcctgcgaagagttgcgaggcagcgg | |
| | cctggtggaacacgcctgggtcaatgatgacctggtgcattgcaaacgct | |
| | agggccttgtggggtcagttccggctgggggttcagcagcccctgctcgg | |
| | atctgttggaccggacagtagtcatggttgatgggctgcctgtatcgagt | |
| | ggtgattttgtgccgagctgccggtcggggagctgttggctggctggtgg | |
| | caggatatattgtggtgtaaacaaattgacgcttagacaacttaataaca | |
| | cattgcggacgtttttaatgtactggggttgaacactct | |
| 105 | gtgccgaattcggatccagcgatctggattttagtactggattttggttt | RC4263 |
| | taggaattagaaattttattgatagaagtattttacaaatacaaatacat | M. Frank |
| | actaagggtttcttatatgctcaacacatgagcgaaaccctataggaacc | SUC9-A |
| | ctaattcccttatctgggaactactcacacattattatggagaaactcga | dCas9-TV |
| | gcttgtcgatcgactctagctagagaagctcactttccaacaaccttagt | Construct |
| | aatttctcccttaacataatgaacaaattcttcaagttgatcagctctag | (RCGG29 |
| | aagcaagtctatcttcttcttgtccaagataagcttgtctagcttcaaga | 96) |
| | ataactggttgatattgagctggaagtctttccatagcccaatcagcagc | |
| | aacatcctttggagcaatctttccagtaacagcagaataccaaattctag | |
| | aaagagtaagaacaacatttctttcatctccagcccaatctggtggagaa | |
| | ttccaaagagtaagagtttcattaagagcttcaaaaagatcttgttctgg | |
| | aactggatcaaaaagttcttcagcagctggtccaacaagagcaacagaat | |
| | gttctctagccttagtaagaagaatagcaagatcaatatcaatagtagct | |
| | ggttcaaaaattccagcaagaatatcatttctttgccattctccaaattg | |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtottt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagatatttgaa | |
| | tatacttaatgttgatgttaaatattatttcatccatcttaaaataagtg | |
| | tcattctatttattttatatagacaaaaaaaagttaataaattaataaaa | |
| | aaacagtaattttataaaactaaccttagaattaatatttcattaaaaaa | |
| | ctaaaattacacttattagaaatattagaaaaaaaatatcactactaaaa | |
| | atattattttttatgacgaacattctaaaatggttatatgaaatcatttt | |
| | agaatgtcatgcgatgacaattgtaaatacaagattgtttaacaaagacg | |
| | attttacaaaaattgtcttaaaaattattgttttatgacggtttgatgaa | |
| | aaccgtcttaaaatgattgtcattctaaaacgattttogtaaaatcctct | |
| | taaaagttagtgcttttaatttttatgtctccctctcctctctcatttct | |
| | ctccctctcactctctactacttcttccgttccaaaataattgttgtatt | |
| | aggttattttacataggtgaagtaataaatagatgaaatataatgataat | |
| | tttagaaaattaatcttatatcatcatttaatacatttatatatagttca | |
| | tcattaataaataaaaaaataattaatgttacatttaaaaactaaaatga | |
| | taaaagtaattaatatacattaagaaactaaaatgacatatttttcttat | |
| | acgacaattataacacgatgagggagtaatgaatattagttaaaattagt | |
| | ttttgatcacctaatttataattttgattcaatttagtcctttagttatt | |
| | ttgggttcaatttaatcctctaatttttaaaattattttttttactttcc | |
| | attccatcacaagttccaaatgaatttgaatttaatcaacttatggtagt | |
| | atatatagaactttgaaagattattatctattgtaaaaaaattcaatctt | |
| | aagattttttttgcataacatgattcctaaactactcatcaggtgtctta | |
| | aagatcagttactagtgtaatggagctagctacttggctcacatttcagg | |
| | tattttacgtaaaagtccttacatgcactgtcgccctaacactttgttta | |
| | tgctaaccttgtcatcaaaatcaaaatgcagaaaagggaaaagaaagaaa | |
| | aaattcaaactggctttcccatccccataaagacttgctaaatataagga | |
| | gaagaagtgaagattatgatcagacatcacgtgggaacccttcttagtta | |
| | aaattgtaataattcaagagagagggtcaagagtcactggaaactctcag | |
| | gtttcaattttcaactgtacaaaataaaaattaaagagatgcaggggtca | |
| | gttaaaggaaataaatcttgtagatataaatatctatatatagattatgg | |
| | ttggataactttttatagaagaagaaaagaaaaacttgtcttataaattt | |
| | aaatcatcttacacatgcatgcacttgaattttatagaaactcttataag | |
| | ctaaatatataaattaattttaatttatgaaaatatatagtttgcttcat | |
| | tttactatttatttacttttgttataagtgtaaacaaggccatattcgaa | |
| | gtaaaaggccaaaaactcaaaatccaagaagcaaaatggtttctttattt | |
| | tttttatgctttaaaaatttgaagcttcggaattttgcgtgcaaactttt | |
| | atgtgcatgtccctctcattgaaattgaatcaaaccccacattaacaaat | |
| | taaagtcactttcgaaaagaccctttgattatctccctctcctttttccc | |
| | atacatgaagaaatggatggtttatggagcatgaaaggtgaacctcaagt | |
| | ctctctctctctcttctctctcatgcttaaattctccaacacgagagtaa | |
| | attttagagtgagtggtaattgtggagggtctatttttctgtcataaata | |
| | cctgtctgctaggccaacccacttccatattttgcatcctttgtacctca | |
| | gttccaggttaacaccaaacaacacatatagtgcagattcataaggtata | |
| | gactttgttaatttgcaccctctcttaccctaaatcttgtgtctattgta | |
| | ttttatctctttccccatggattacaaagatgacgatgataaagactaca | |
| | aggacgacgacgataaaatggcccctaaaaagaagcgcaaggtgggtatt | |
| | catggggtgcctgcagctggaatggataagaagtactccatcggactggc | |
| | tatcggaactaactccgtgggatgggctgtgatcactgatgagtacaagg | |
| | tgccatccaagaagttcaaggtgctgggaaacactgatagacactccatc | |
| | aagaagaacctgatcggagctctgctgttcgattccggagagactgctga | |
| | ggctactagactgaagagaactgctagaagaagatacactagaagaaaga | |
| | acagaatctgctacctgcaagagatcttctccaacgagatggctaaggtg | |
| | gatgattccttcttccacagactggaggagtccttcctggtggaggagga | |
| | taagaagcacgagagacacccaatcttcggaaacatcgtggatgaggtgg | |
| | cttaccacgagaagtacccaactatctaccacctgagaaagaagctggtg | |
| | gattccactgataaggctgatctgagactgatctacctggctctggctca | |
| | catgatcaagttcagaggacacttcctgatcgagggagatctgaacccag | |
| | ataactccgatgtggataagctgttcatccaactggtgcaaacttacaac | |
| | caactgttcgaggagaacccaatcaacgcttccggagtggatgctaaggc | |
| | tatcctgtccgctagactgtccaagtccaggagactggagaacctgatcg | |
| | ctcaactgccaggagagaagaagaacggactgttcggaaacctgatcgct | |
| | ctgtccctgggactgactccaaacttcaagtccaacttcgatctggctga | |
| | ggatgctaagctgcaactgtccaaggatacttacgatgatgatctggata | |
| | acctgctggctcaaatcggagatcaatacgctgatctgttcctggctgct | |
| | aagaacctgtccgatgctatcctgctgtccgatatcctgagagtgaacac | |
| | tgagatcactaaggctccactgtccgcttccatgatcaagagatacgatg | |
| | agcaccaccaagatctgactctgctgaaggctctggtgagacaacaactg | |
| | ccagagaagtacaaggagatcttcttcgatcaatccaagaacggatacgc | |
| | tggatacatcgatggaggagcttcccaagaggagttctacaagttcatca | |
| | agccaatcctggagaagatggatggaactgaggagctgctggtgaagctg | |
| | aacagagaggatctgctgagaaagcaaagaactttcgataacggatccat | |
| | cccacaccaaatccacctgggagagctgcacgctatcctgaggagacaag | |
| | aggatttctacccattcctgaaggataacagagagaagatcgagaagatc | |
| | ctgactttcagaatcccatactacgtgggaccactggctagaggaaactc | |
| | cagattcgcttggatgactagaaagtccgaggagactatcactccatgga | |
| | acttcgaggaggtaagtttctgcttctacctttgatatatatataataat | |
| | tatcattaattagtagtaatataatatttcaaatatttttttcaaaataa | |
| | aagaatgtagtatatagcaattgcttttctgtagtttataagtgtgtata | |
| | ttttaatttataacttttctaatatatgaccaaaatttgttgatgtgcag | |
| | gtggtggataagggagcttccgctcaatccttcatcgagagaatgactaa | |
| | cttcgataagaacctgccaaacgagaaggtgctgccaaagcactccctgc | |
| | tgtacgagtacttcactgtgtacaacgagctgactaaggtgaagtacgtg | |
| | actgagggaatgagaaagccagctttcctgtccggagagcaaaagaaggc | |
| | tatcgtggatctgctgttcaagactaacagaaaggtgactgtgaagcaac | |
| | tgaaggaggattacttcaagaagatcgagtgcttcgattccgtggagatc | |
| | tccggagtggaggatagattcaacgcttccctgggaacttaccacgatct | |
| | gctgaagatcatcaaggataaggatttcctggataacgaggagaacgagg | |
| | atatcctggaggatatcgtgctgactctgactctgttcgaggatagagag | |
| | atgatcgaggagagacttaagacttacgctcacctgttcgatgataaggt | |
| | gatgaagcaactgaagagaagaagatacactggatggggtagactgtcca | |
| | gaaagctgatcaacggaatcagagataagcaatccggaaagactatcctg | |
| | gatttcctgaagtccgatggattcgctaacagaaacttcatgcaactgat | |
| | ccacgatgattccctgactttcaaggaggatatccaaaaggctcaagtgt | |
| | ccggacaaggagattccctgcacgagcacatcgctaacctggctggatcc | |
| | ccagctatcaagaagggaatcctgcaaactgtgaaggtggtggatgagct | |
| | ggtgaaggtgatgggtagacacaagccagagaacatcgtgatcgagatgg | |
| | ctagagagaaccaaactactcaaaagggacaaaagaactccagagagaga | |
| | atgaagagaatcgaggagggaatcaaggagctgggatcccaaatcctgaa | |
| | ggagcacccagtggagaacactcaactgcaaaacgagaagctgtacctgt | |
| | actacctgcaaaacggaagagatatgtacgtggatcaagagctggatatc | |
| | aacagactgtccgattacgatgtggatgcaatcgtgccacaatccttcct | |
| | gaaggatgattccatcgataacaaggtgctgactagatccgataagaaca | |
| | gaggaaagtccgataacgtgccatccgaggaggtggtgaagaagatgaag | |
| | aactactggagacaactgctgaacgctaagctgatcactcaaagaaagtt | |
| | cgataacctgactaaggctgagagaggaggactgtccgagctggataagg | |
| | ctggattcatcaagagacaactggtggagactagacaaatcactaagcac | |
| | gtggctcaaatcctggattccagaatgaacactaagtacgatgagaacga | |
| | taagctgatcagagaggtgaaggtgatcactctgaagtccaagctggtgt | |
| | ccgatttcagaaaggatttccaattctacaaggtgagagagatcaacaac | |
| | taccaccacgctcacgatgcttacctgaacgctgtggtgggaactgctct | |
| | gatcaagaagtacccaaagctggagtccgagttcgtgtacggagattaca | |
| | aggtgtacgatgtgagaaagatgatcgctaagtccgagcaagagatcgga | |
| | aaggctactgctaagtacttcttctactccaacatcatgaacttcttcaa | |
| | gactgagatcactctggctaacggagagatcagaaaaagaccactgatcg | |
| | agactaacggagagactggagagatcgtgtgggataagggaagagatttc | |
| | gctactgtgagaaaggtgctgtccatgccacaagtgaacatcgtgaaaaa | |
| | gactgaggtgcaaactggaggattctccaaggagtccatcctgccaaaga | |
| | gaaactccgataagctgatcgctagaaagaaggattgggatccaaagaag | |
| | tacggaggattcgattccccaactgtggcttactccgtgctggtggtggc | |
| | taaggtggagaagggaaagtccaagaagctgaagtccgtgaaggagctgc | |
| | tgggaatcactatcatggagagatcctccttcgagaagaacccaatcgat | |
| | ttcctggaggctaagggatacaaggaggtgaagaaggatctgatcatcaa | |
| | gctgccaaagtactccctgttcgagctggagaacggaagaaagagaatgc | |
| | tggcttccgctggagagctgcaaaagggaaacgagctggctctgccatcc | |
| | aagtacgtgaacttcctgtacctggcttcccactacgagaagctgaaggg | |
| | atccccagaggataacgagcaaaagcaactgttcgtggagcaacacaagc | |
| | actacctggatgagatcatcgagcaaatctccgagttctccaagagagtg | |
| | atcctggctgatgctaacctggataaggtgctgtccgcttacaacaagca | |
| | cagagataagccaatcagagagcaagctgagaacatcatccacctgttca | |
| | ctctgactaacctgggagctccagctgctttcaagtacttcgatactact | |
| | atcgatagaaagagatacacttccactaaggaggtgctggatgctactct | |
| | gatccaccaatccatcactggactgtacgagactagaatcgatctgtccc | |
| | aactgggaggagatagttcgaagaggcctgccgccacaaagaaagccggc | |
| | caggcaaagaaaaaaaaacccaggggagggagtggtggactcctcgaccc | |
| | tggcactccaatggacgccgatctcgtagcatcatctaccgtcgtgtggg | |
| | agcaggacgccgacccttttgctggcacagctgatgatttcccagctttt | |
| | aatgaggaagaattggcttggctgatggagcttctcccacaaggtggtag | |
| | cggcgggctcctcgatcccggtactcccatggacgcagatcttgttgcta | |
| | gctccactgttgtatgggagcaggacgccgatcccttcgctggaaccgcc | |
| | gatgatttcccagcattcaacgaggaagaacttgcatggctcatggagct | |
| | cttgccccaagctcgcggtggatctgggggtttgttggatcccggaaccc | |
| | caatggacgcagacctcgtggcttctagtaccgtagtatgggaacaggac | |
| | gctgaccctttcgccggcaccgcagatgacttcccagcattcaacgagga | |
| | ggaactggcctggctgatggaacttctgcctcagggtggatccggaggcc | |
| | tcttggaccccggtacccctatggacgctgacctggtcgcatcttctacc | |
| | gtggtttgggagcaagatgccgacccattcgcaggaacagctgatgactt | |
| | ccccgcattcaatgaagaagagctcgcatggctgatggaattgcttccac | |
| | aagccagaggcggctctggagggcttcttgatcccggaacacccatggat | |
| | gccgacctcgtggcctctagcaccgtggtatgggaacaagacgcagatcc | |
| | attcgcagggaccgcagacgatttccccgcttttaacgaggaggagcttg | |
| | cttggctcatggagctgctgcctcagggtggatccgggggacttctcgac | |
| | ccaggcacacccatggatgccgatctcgtcgccagttctaccgttgtctg | |
| | ggagcaagacgcagaccctttcgccggaactgcagacgacttccctgcct | |
| | ttaatgaagaagaactggcttggctcatggaactgctcccccaggctaga | |
| | gggggtagcggcgggggcggcagcggaggtgatgcacttgacgacttcga | |
| | tcttgatatgcttggcagcgacgcacttgatgacttcgatttggatatgc | |
| | tggggtccgatgctcttgacgattttgatttggacatgttgggttcagac | |
| | gcactcgatgatttcgatttggacatgctggcacgtggaagcgatgccct | |
| | tgatgactttgacctggatatgctcgggtccgatgctttggacgacttcg | |
| | acctcgacatgttgggaagtgatgctcttgatgatttcgatcttgatatg | |
| | cttgggtccgacgctctcgatgattttgacctcgatatgctctgagcttg | |
| | tcaagcagatcgttcaaacatttggcaataaagtttcttaagattgaatc | |
| | ctgttgccggtcttgcgatgattatcatataatttctgttgaattacgtt | |
| | aagcatgtaataattaacatgtaatgcatgacgttatttatgagatgggt | |
| | ttttatgattagagtcccgcaattatacatttaatacgcgatagaaaaca | |
| | aaatatagcgcgcaaactaggataaattatcgcgcgcggtgtcatctatg | |
| | ttactagatcgacgctttacgaattcccatggggaggacgtcgttgtggt | |
| | tggtgctttccttacattctgagcctctttccttctaatccactcatctg | |
| | catcttcttgtgtccttactaatacctcattggttccaaattccctccct | |
| | ttaagcaccagctcgtttctgttcttccacagcctcccaagtatccaagg | |
| | gactaaagcctccacattcttcagatcaggatattcttgtttaagatgtt | |
| | gaactctatggaggtttgtatgaactgatgatctaggaccggataagttc | |
| | ccttcttcatagcgaacttattcaaagaatgttttgtgtatcattcttgt | |
| | tacattgttattaatgaaaaaatattattggtcattggactgaacacgag | |
| | tgttaaatatggaccaggccccaaataagatccattgatatatgaattaa | |
| | ataacaagaataaatcgagtcaccaaaccacttgccttttttaacgagac | |
| | ttgttcaccaacttgatacaaaagtcattatcctatgcaaatcaataatc | |
| | atacaaaaatatccaataacactaaaaaaattaaaagaaatggataattt | |
| | cacaatatgttatacgataaagaagttacttttccaagaaattcactgat | |
| | tttataagcccacttgcattagataaatggcaaaaaaaaacaaaaaggaa | |
| | aagaaataaagcacgaagaattctagaaaatacgaaatacgcttcaatgc | |
| | agtgggacccacggttcaattattgccaattttcagctccaccgtatatt | |
| | taaaaaataaaacgataatgctaaaaaaatataaatcgtaacgatcgtta | |
| | aatctcaacggctggatcttatgacgaccgttaaggaaattgtggttgtc | |
| | ggacgaagtccagtaataaacggcgtcaaagtggttgcagccggcacaca | |
| | cgagtcgtgtttatcaactcaaagcacaaatacttttcctcaacctaaaa | |
| | ataaggcaattagccaaaaacaactttgcgtgtaaacaacgctcaataca | |
| | cgtgtcattttattattagctattgcttcaccgccttagctttctcgtga | |
| | cctagtcgtcctcgtcttttcttcttcttcttctataaaacaatacccaa | |
| | agagctcttcttcttcacaattcagatttcaatttctcaaaatcttaaaa | |
| | actttctctcaattctctctaccgtgatcaaggtaaatttctgtgttcct | |
| | tattctctcaaaatcttcgattttgttttcgttcgatcccaatttcgtat | |
| | atgttctttggtttagattctgttaatcttagatcgtagacgattttctg | |
| | ggtttgatcgttagatatcatcttaattctcgattagggtttcatagata | |
| | tcatccgatttgttcaaataatttgagttttgtcgaataattactcttcg | |
| | atttgtgatttctatctagatctggtgttagtttctagtttgtgcgatcg | |
| | aatttgtcgattaatctgagtttttctgattaacagatgcagatcttaat | |
| | gacaaagcaccagtggtctagtggtagaatagtaccctgccacggtacag | |
| | acccgggttcgattcccggctggtgcatatcgtatagtaattagctggtt | |
| | ttagagctagaaatagcaagttaaaataaggctagtccgttatcaacttg | |
| | aaaaagtggcaccgagtcggtgcaacaaagcaccagtggtctagtggtag | |
| | aatagtaccctgccacggtacagacccgggttcgattcccggctggtgca | |
| | catcatttacgtaaacgtttgttttagagctagaaatagcaagttaaaat | |
| | aaggctagtccgttatcaacttgaaaaagtggcaccgagtcggtgcaaca | |
| | aagcaccagtggtctagtggtagaatagtaccctgccacggtacagaccc | |
| | gggttcgattcccggctggtgcaagggttgaagagtttatatagttttag | |
| | agctagaaatagcaagttaaaataaggctagtccgttatcaacttgaaaa | |
| | agtggcaccgagtcggtgccaacaaagcaccagtggtctagtggtagaat | |
| | agtaccctgccacggtacagacccgggttcgattcccggctggtgcagct | |
| | tctctagctagagtcgatcgacaagctcgagtttctccataataatgtgt | |
| | gagtagttcccagataagggaattagggttcctatagggtttcgctcatg | |
| | tgttgagcatataagaaacccttagtatgtatttgtatttgtaaaatact | |
| | tctatcaataaaatttctaattcctaaaaccaaaatccagtactaaaatc | |
| | cagatcgctcagagaggatgcacatgtgaccgagggacacgaagtgatcc | |
| | gtttaaactatcagtgtttgacaggatatattggcgggtaaacctaagag | |
| | aaaagagcgtttattagaataatcggatatttaaaagggcgtgaaaaggt | |
| | ttatccgttcgtccatttgtatgtgccagccgcctttgcgacgctcaccg | |
| | ggctggttgccctcgccgctgggctggcggccgtctatggccctgcaaac | |
| | gcgccagaaacgccgtcgaagccgtgtgcgagacaccgeggccgccggcg | |
| | ttgtggatacctcgcggaaaacttggccctcactgacagatgaggggcgg | |
| | acgttgacacttgaggggccgactcacccggcgcggcgttgacagatgag | |
| | gggcaggctcgatttcggccggcgacgtggagctggccagcctcgcaaat | |
| | cggcgaaaacgcctgattttacgcgagtttcccacagatgatgtggacaa | |
| | gcctggggataagtgccctgcggtattgacacttgaggggcgcgactact | |
| | gacagatgaggggcgcgatccttgacacttgaggggcagagtgctgacag | |
| | atgaggggcgcacctattgacatttgaggggctgtccacaggcagaaaat | |
| | ccagcatttgcaagggtttccgcccgtttttcggccaccgctaacctgtc | |
| | ttttaacctgottttaaaccaatatttataaaccttgtttttaaccaggg | |
| | ctgcgccctgtgcgcgtgaccgcgcacgccgaaggggggtgccccccctt | |
| | ctegaaccctcccggcccgctaacgcgggcctcccatccccccaggggct | |
| | gcgcccctcggccgcgaacggcctcaccccaaaaatggcagcgctggcca | |
| | attcccgagtgcgcggaacccctatttgtttatttttctaaatacattca | |
| | aatatgtatccgctcatgagacaataaccctgataaatgcttcaataata | |
| | ttgaaaaaggaagagtatggctaaaatgagaatatcaccggaattgaaaa | |
| | aactgatcgaaaaataccgctgcgtaaaagatacggaaggaatgtctcct | |
| | gctaaggtatataagctggtgggagaaaatgaaaacctatatttaaaaat | |
| | gacggacagccggtataaagggaccacctatgatgtggaacgggaaaagg | |
| | acatgatgctatggctggaaggaaagctgcctgttccaaaggtcctgcac | |
| | tttgaacggcatgatggctggagcaatctgctcatgagtgaggccgatgg | |
| | cgtcctttgctcggaagagtatgaagatgaacaaagccctgaaaagatta | |
| | tcgagctgtatgcggagtgcatcaggctctttcactccatcgacatatcg | |
| | gattgtccctatacgaatagcttagacagccgcttagccgaattggatta | |
| | cttactgaataacgatctggccgatgtggattgcgaaaactgggaagagg | |
| | acactccatttaaagatccgcgcgagctgtatgattttttaaagacggaa | |
| | aagcccgaagaggaacttgtcttttcccacggcgacctgggagacagcaa | |
| | catctttgtgaaagatggcaaagtaagtggctttattgatcttgggagaa | |
| | gcggcagggggacaagtggtatgacattgccttctgcgtccggtcgctca | |
| | gggaggatatcggggaagaacagtatgtcgagctattttttgacttactg | |
| | gggatcaagcctgattgggagaaaataaaatattatattttactggatga | |
| | attgttttagctgtcagaccaagtttactcatatatactttagattgatt | |
| | taaaacttcatttttaatttaaaaggatctaggtgaagatcctttttgat | |
| | aatctcatgaccaaaatcccttaacgtgagttttcgttccactgagcgtc | |
| | agaccccgtagaaaagatcaaaggatcttcttgagatcctttttttctgc | |
| | gcgtaatctgctgcttgcaaacaaaaaaaccaccgctaccagcggtggtt | |
| | tgtttgccggatcaagagctaccaactctttttccgaaggtaactggctt | |
| | cagcagagcgcagataccaaatactgtccttctagtgtagccgtagttag | |
| | gccaccacttcaagaactctgtagcaccgcctacatacctcgctctgcta | |
| | atcctgttaccagtggctgctgccagtggcgataagtcgtgtcttaccgg | |
| | gttggactcaagacgatagttaccggataaggcgcagcggtcgggctgaa | |
| | cggggggttcgtgcacacagcccagcttggagcgaacgacctacaccgaa | |
| | ctgagatacctacagcgtgagctatgagaaagcgccacgcttcccgaagg | |
| | gagaaaggcggacaggtatccggtaagcggcagggtcggaacaggagagc | |
| | gcacgagggagcttccagggggaaacgcctggtatctttatagtcctgtc | |
| | gggtttcgccacctctgacttgagcgtcgatttttgtgatgctcgtcagg | |
| | ggggcggagcctatggaaaaacgccagcaacgcggcctttttacggttcc | |
| | tggcagatcctagatgtggcgcaacgatgccggcgacaagcaggagcgca | |
| | ccgacttcttccgcatcaagtgttttggctctcaggccgaggcccacggc | |
| | aagtatttgggcaaggggtcgctggtattcgtgcagggcaagattcggaa | |
| | taccaagtacgagaaggacggccagacggtctacgggaccgacttcattg | |
| | ccgataaggtggattatctggacaccaaggcaccaggcgggtcaaatcag | |
| | gaataagggcacattgccccggcgtgagtcggggcaatcccgcaaggagg | |
| | gtgaatgaatcggacgtttgaccggaaggcatacaggcaagaactgatcg | |
| | acgcggggttttccgccgaggatgccgaaaccategcaagccgcaccgtc | |
| | atgcgtgcgccccgcgaaaccttccagtccgtcggctcgatggtccagca | |
| | agctacggccaagatcgagcgcgacagcgtgcaactggctccccctgccc | |
| | tgcccgcgccatcggccgccgtggagcgttcgcgtcgtcttgaacaggag | |
| | gcggcaggtttggcgaagtcgatgaccatcgacacgcgaggaactatgac | |
| | gaccaagaagcgaaaaaccgccggcgaggacctggcaaaacaggtcagcg | |
| | aggccaagcaggccgcgttgctgaaacacacgaagcagcagatcaaggaa | |
| | atgcagctttccttgttcgatattgcgccgtggccggacacgatgcgagc | |
| | gatgccaaacgacacggcccgctctgccctgttcaccacgcgcaacaaga | |
| | aaatcccgcgcgaggcgctgcaaaacaaggtcattttccacgtcaacaag | |
| | gacgtgaagatcacctacaccggcgtcgagctgcgggccgacgatgacga | |
| | actggtgtggcagcaggtgttggagtacgcgaagcgcacccctatcggcg | |
| | agccgatcaccttcacgttctacgagctttgccaggacctgggctggtcg | |
| | atcaatggccggtattacacgaaggccgaggaatgcctgtcgcgcctaca | |
| | ggcgacggcgatgggcttcacgtccgaccgcgttgggcacctggaatcgg | |
| | tgtcgctgctgcaccgcttccgcgtcctggaccgtggcaagaaaacgtcc | |
| | cgttgccaggtcctgatcgacgaggaaatcgtcgtgctgtttgctggcga | |
| | ccactacacgaaattcatatgggagaagtaccgcaagctgtcgccgacgg | |
| | cccgacggatgttcgactatttcagctcgcaccgggagccgtacccgctc | |
| | aagctggaaaccttccgcctcatgtgcggatcggattccacccgcgtgaa | |
| | gaagtggcgcgagcaggtcggcgaagcctgcgaagagttgcgaggcagcg | |
| | gcctggtggaacacgcctgggtcaatgatgacctggtgcattgcaaacgc | |
| | tagggccttgtggggtcagttccggctgggggttcagcagcccctgctcg | |
| | gatctgttggaccggacagtagtcatggttgatgggctgcctgtatcgag | |
| | tggtgattttgtgccgagctgccggtcggggagctgttggctggctggtg | |
| | gcaggatatattgtggtgtaaacaaattgacgcttagacaacttaataac | |
| | acattgcggacgtttttaatgtactggggttgaacactct | |
| 106 | gtgccgaattcggatccagcgatctggattttagtactggattttggttt | RC4262 |
| | taggaattagaaattttattgatagaagtattttacaaatacaaatacat | M. Frank |
| | actaagggtttcttatatgctcaacacatgagcgaaaccctataggaacc | AINTEGU |
| | ctaattcccttatctgggaactactcacacattattatggagaaactcga | MENTA-B |
| | gcttgtcgatcgactctagctagagaagctcactttccaacaaccttagt | dCas9-TV |
| | aatttctcccttaacataatgaacaaattcttcaagttgatcagctctag | Construct |
| | aagcaagtctatcttcttcttgtccaagataagcttgtctagcttcaaga | (RCGG29 |
| | ataactggttgatattgagctggaagtctttccatagcccaatcagcagc | 95) |
| | aacatcctttggagcaatctttccagtaacagcagaataccaaattctag | |
| | aaagagtaagaacaacatttctttcatctccagcccaatctggtggagaa | |
| | ttccaaagagtaagagtttcattaagagcttcaaaaagatcttgttctgg | |
| | aactggatcaaaaagttcttcagcagctggtccaacaagagcaacagaat | |
| | gttctctagccttagtaagaagaatagcaagatcaatatcaatagtagct | |
| | ggttcaaaaattccagcaagaatatcattttttgccattctccaaattga | |
| | agttctctcttagctggatatctccatggaataatatcatcatgaacaac | |
| | aatagtaacttcaacagctctaagaatttcagattctcctggagaagcag | |
| | aagtttcaagaagatcattaataagagctcttctagtagtttcatcaagt | |
| | ctaacagtaacagtaacaagaagatcaatatcagaatgtggcttaagtcc | |
| | tccatcaacagcagatccataaagatgaacagcaagaagagtaggttcaa | |
| | gatgtctttcaataactccaacaacttcagaaagttgagtagaaacttca | |
| | gcaataacagcttctctcatgcaagcagtagcaacagaagcagaaattct | |
| | gaaagaacagaacttttgcatgaaaatagaatccttcttaagaacaagca | |
| | tagaattagcagagttcttaagcttcttagatccgaaaacaagaaaagaa | |
| | gatgacttaggaacttgtggcttatggaaattagagttaggattaagagt | |
| | ttgaattccttgagccatattgttaatttgagccattgtatcgataattg | |
| | taaatgtaattgtaatgttgtttgttgtttgttgttgttggtaattgttg | |
| | taaaaatgagctcttatactcgagcgtgtcctctccaaatgaaatgaact | |
| | tccttatatagaggaagggtcttgcgaaggatagtgggattgtgcgtcat | |
| | cccttacgtcagtggagatgtcacatcaatccacttgctttgtagacgtg | |
| | gttggaacctcttctttttccacgatgctcctcgtgggtgggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagtgttcaaaa | |
| | ataaaaagtgattttgagatataatatacatacgacaattaatacaatat | |
| | tatttttgtgtctatccctctcttagaccctattatttatcacatttgaa | |
| | attcagaattttctttttctctcgttgttgtacaaatatctaatcgttaa | |
| | ttattattaatgcattaaatatttaaaatataattaatgatattaatttt | |
| | cctagcgtaagactaataataaatatcgacattatgcgagattatgacat | |
| | gatgttatgttaatagtacagaataaagtaaaaaaatatatataacaata | |
| | ataatttaaaatatttaaaatgacatataaagtagttttctaaaacttca | |
| | gtgattagttcaatttattatttcgcaacaattatgatggttaattaagg | |
| | tgaattagaagaagaactgaaaattaagataattaaggagaacaaatgag | |
| | cacttcaaaatttgctattaagccgactctgctagccatgcatgttggat | |
| | gtaacttcacccgattgatgggtccttaattataatttttcttatgataa | |
| | aaaataaattgctactaaggcgtactgaactattcgaaatgacaacacta | |
| | caaggcctcgacaacgttaacttttttccaatttagtggggcgttacaag | |
| | ctaaagcttttttatttttgaactttaaattgctctttcactttcattta | |
| | ataacgcctaaacctagctacgagcaatataacctgggtgtatatagcta | |
| | tatatatcatatgcagagcaatctgtaaccccaaggataaaacgttgtgt | |
| | tgcttattatagaaatcacagttttaaatggaacgagtgacagagtttgt | |
| | ttgtttctaaataggataggaggcatttaatcggaaattggacaggagaa | |
| | agttgttttctttctgcaaattatgcctccagaacttgttttacggtaag | |
| | aaaatttacaccaaatttacaagtattggttctgaacgctacttgcggaa | |
| | attttatgccgccagtgacttcttggcactttaaataagttaattagtta | |
| | atcatttgaaaatatattaacaagaagaatttttttctagtgtaatcaag | |
| | aggtattttaagcttctatcttacactcaattaatcttctattctcctat | |
| | tgattggtctattttaaagtaaagtaatatattcaattacgacttcatgc | |
| | taaattcaaagattataacagtaaaaacaatttttattcacctctattca | |
| | aactgccagtttagcgcaagtgtttatttttctgataaacttgcggccgc | |
| | acaaaacccagcattgcttcaatgtgcataagcatgggtacggtggtggt | |
| | gtttctccaacctccatttcagattgtcttgatttgtaattcaaacacac | |
| | acttagtcttaaaatcatattttctcatatctcaattgtgatttttccaa | |
| | atgcgttgtgtgtaggcgaagctaagtttaggttattgctcaagtgccac | |
| | aagtttgagagggttttactgtcttttttatttaatctagaaatatgatt | |
| | tttggacacgtctacttgttacattccgtttacttatggtagtatttgac | |
| | taataaagacattaagattatagtagcatttgactaataaagacacgcgt | |
| | acgtaggtcactattttattggctaagtaaaaaaaaaaaaaaacacgaaa | |
| | aaactttcaaacaaaggggaagcggagaaatcagcctaacttgatattag | |
| | ctgtacaaaatacataaatagctgtcggaaataattatctcttgcagtat | |
| | atatatagaaagtagcagactctaaaatataacatgaagatgtgattaag | |
| | catttattccattatatggcccataaacatgcatgatgcaaatagaagga | |
| | tgtagggtaaagttgccctaagaaacgaattgaactgcggtcgttgcggt | |
| | atgagaaagagagagagaaaagtgagtgaaggaaggtgggagaatggttt | |
| | ccgtgacggagtaggacggaaagtaagggtccccacagtgcacatgggag | |
| | cggatttgacgatgagtcgagaaagagcacaagacgtttatgacaaagcc | |
| | tgcaatagctttcgttaatttggcaaaagacccctaccatttgcggcaat | |
| | gcacctccccgttctattatttctctttccctcattcattatatccccta | |
| | ttctctgccccagatcgatcgaacaagtgaattagctagaagcattgaag | |
| | gacgtagctgctccatggattacaaagatgacgatgataaagactacaag | |
| | gacgacgacgataaaatggcccctaaaaagaagcgcaaggtgggtattca | |
| | tggggtgcctgcagctggaatggataagaagtactccatcggactggcta | |
| | tcggaactaactccgtgggatgggctgtgatcactgatgagtacaaggtg | |
| | ccatccaagaagttcaaggtgctgggaaacactgatagacactccatcaa | |
| | gaagaacctgatcggagctctgctgttcgattccggagagactgctgagg | |
| | ctactagactgaagagaactgctagaagaagatacactagaagaaagaac | |
| | agaatctgctacctgcaagagatcttctccaacgagatggctaaggtgga | |
| | tgattccttcttccacagactggaggagtccttcctggtggaggaggata | |
| | agaagcacgagagacacccaatcttcggaaacatcgtggatgaggtggct | |
| | taccacgagaagtacccaactatctaccacctgagaaagaagctggtgga | |
| | ttccactgataaggctgatctgagactgatctacctggctctggctcaca | |
| | tgatcaagttcagaggacacttcctgatcgagggagatctgaacccagat | |
| | aactccgatgtggataagctgttcatccaactggtgcaaacttacaacca | |
| | actgttcgaggagaacccaatcaacgcttccggagtggatgctaaggcta | |
| | tcctgtccgctagactgtccaagtccaggagactggagaacctgatcgct | |
| | caactgccaggagagaagaagaacggactgttcggaaacctgatcgctct | |
| | gtccctgggactgactccaaacttcaagtccaacttcgatctggctgagg | |
| | atgctaagctgcaactgtccaaggatacttacgatgatgatctggataac | |
| | ctgctggctcaaatcggagatcaatacgctgatctgttcctggctgctaa | |
| | gaacctgtccgatgctatcctgctgtccgatatcctgagagtgaacactg | |
| | agatcactaaggctccactgtccgcttccatgatcaagagatacgatgag | |
| | caccaccaagatctgactctgctgaaggctctggtgagacaacaactgcc | |
| | agagaagtacaaggagatcttcttcgatcaatccaagaacggatacgctg | |
| | gatacatcgatggaggagcttcccaagaggagttctacaagttcatcaag | |
| | ccaatcctggagaagatggatggaactgaggagctgctggtgaagctgaa | |
| | cagagaggatctgctgagaaagcaaagaactttcgataacggatccatcc | |
| | cacaccaaatccacctgggagagctgcacgctatcctgaggagacaagag | |
| | gatttctacccattcctgaaggataacagagagaagatcgagaagatcct | |
| | gactttcagaatcccatactacgtgggaccactggctagaggaaactcca | |
| | gattcgcttggatgactagaaagtccgaggagactatcactccatggaac | |
| | ttcgaggaggtaagtttctgcttctacctttgatatatatataataatta | |
| | tcattaattagtagtaatataatatttcaaatatttttttcaaaataaaa | |
| | gaatgtagtatatagcaattgcttttctgtagtttataagtgtgtatatt | |
| | ttaatttataacttttctaatatatgaccaaaatttgttgatgtgcaggt | |
| | ggtggataagggagcttccgctcaatccttcatcgagagaatgactaact | |
| | tcgataagaacctgccaaacgagaaggtgctgccaaagcactccctgctg | |
| | tacgagtacttcactgtgtacaacgagctgactaaggtgaagtacgtgac | |
| | tgagggaatgagaaagccagctttcctgtccggagagcaaaagaaggcta | |
| | tcgtggatctgctgttcaagactaacagaaaggtgactgtgaagcaactg | |
| | aaggaggattacttcaagaagatcgagtgcttcgattccgtggagatctc | |
| | cggagtggaggatagattcaacgcttccctgggaacttaccacgatctgc | |
| | tgaagatcatcaaggataaggatttcctggataacgaggagaacgaggat | |
| | atcctggaggatatcgtgctgactctgactctgttcgaggatagagagat | |
| | gatcgaggagagacttaagacttacgctcacctgttcgatgataaggtga | |
| | tgaagcaactgaagagaagaagatacactggatggggtagactgtccaga | |
| | aagctgatcaacggaatcagagataagcaatccggaaagactatcctgga | |
| | tttcctgaagtccgatggattcgctaacagaaacttcatgcaactgatcc | |
| | acgatgattccctgactttcaaggaggatatccaaaaggctcaagtgtcc | |
| | ggacaaggagattccctgcacgagcacatcgctaacctggctggatcccc | |
| | agctatcaagaagggaatcctgcaaactgtgaaggtggtggatgagctgg | |
| | tgaaggtgatgggtagacacaagccagagaacatcgtgatcgagatggct | |
| | agagagaaccaaactactcaaaagggacaaaagaactccagagagagaat | |
| | gaagagaatcgaggagggaatcaaggagctgggatcccaaatcctgaagg | |
| | agcacccagtggagaacactcaactgcaaaacgagaagctgtacctgtac | |
| | tacctgcaaaacggaagagatatgtacgtggatcaagagctggatatcaa | |
| | cagactgtccgattacgatgtggatgcaatcgtgccacaatccttcctga | |
| | aggatgattccatcgataacaaggtgctgactagatccgataagaacaga | |
| | ggaaagtccgataacgtgccatccgaggaggtggtgaagaagatgaagaa | |
| | ctactggagacaactgctgaacgctaagctgatcactcaaagaaagttcg | |
| | ataacctgactaaggctgagagaggaggactgtccgagctggataaggct | |
| | ggattcatcaagagacaactggtggagactagacaaatcactaagcacgt | |
| | ggctcaaatcctggattccagaatgaacactaagtacgatgagaacgata | |
| | agctgatcagagaggtgaaggtgatcactctgaagtccaagctggtgtcc | |
| | gatttcagaaaggatttccaattctacaaggtgagagagatcaacaacta | |
| | ccaccacgctcacgatgcttacctgaacgctgtggtgggaactgctctga | |
| | tcaagaagtacccaaagctggagtccgagttcgtgtacggagattacaag | |
| | gtgtacgatgtgagaaagatgatcgctaagtccgagcaagagatcggaaa | |
| | ggctactgctaagtacttcttctactccaacatcatgaacttcttcaaga | |
| | ctgagatcactctggctaacggagagatcagaaaaagaccactgatcgag | |
| | actaacggagagactggagagatcgtgtgggataagggaagagatttcgc | |
| | tactgtgagaaaggtgctgtccatgccacaagtgaacatcgtgaaaaaga | |
| | ctgaggtgcaaactggaggattctccaaggagtccatcctgccaaagaga | |
| | aactccgataagctgatcgctagaaagaaggattgggatccaaagaagta | |
| | cggaggattcgattccccaactgtggcttactccgtgctggtggtggcta | |
| | aggtggagaagggaaagtccaagaagctgaagtccgtgaaggagctgctg | |
| | ggaatcactatcatggagagatcctccttcgagaagaacccaatcgattt | |
| | cctggaggctaagggatacaaggaggtgaagaaggatctgatcatcaagc | |
| | tgccaaagtactccctgttcgagctggagaacggaagaaagagaatgctg | |
| | gcttccgctggagagctgcaaaagggaaacgagctggctctgccatccaa | |
| | gtacgtgaacttcctgtacctggcttcccactacgagaagctgaagggat | |
| | ccccagaggataacgagcaaaagcaactgttcgtggagcaacacaagcac | |
| | tacctggatgagatcatcgagcaaatctccgagttctccaagagagtgat | |
| | cctggctgatgctaacctggataaggtgctgtccgcttacaacaagcaca | |
| | gagataagccaatcagagagcaagctgagaacatcatccacctgttcact | |
| | ctgactaacctgggagctccagctgctttcaagtacttcgatactactat | |
| | cgatagaaagagatacacttccactaaggaggtgctggatgctactctga | |
| | tccaccaatccatcactggactgtacgagactagaatcgatctgtcccaa | |
| | ctgggaggagatagttcgaagaggcctgccgccacaaagaaagccggcca | |
| | ggcaaagaaaaaaaaacccaggggagggagtggtggactcctcgaccctg | |
| | gcactccaatggacgccgatctcgtagcatcatctaccgtcgtgtgggag | |
| | caggacgccgacccttttgctggcacagctgatgatttcccagcttttaa | |
| | tgaggaagaattggcttggctgatggagcttctcccacaaggtggtageg | |
| | gcgggctcctcgatcccggtactcccatggacgcagatcttgttgctagc | |
| | tccactgttgtatgggagcaggacgccgatcccttcgctggaaccgccga | |
| | tgatttcccagcattcaacgaggaagaacttgcatggctcatggagctct | |
| | tgccccaagctcgcggtggatctgggggtttgttggatcccggaacccca | |
| | atggacgcagacctcgtggcttctagtaccgtagtatgggaacaggacgc | |
| | tgaccctttcgccggcaccgcagatgacttcccagcattcaacgaggagg | |
| | aactggcctggctgatggaacttctgcctcagggtggatccggaggcctc | |
| | ttggaccccggtacccctatggacgctgacctggtcgcatcttctaccgt | |
| | ggtttgggagcaagatgccgacccattcgcaggaacagctgatgacttcc | |
| | ccgcattcaatgaagaagagctcgcatggctgatggaattgcttccacaa | |
| | gccagaggcggctctggagggcttcttgatcccggaacacccatggatgc | |
| | cgacctcgtggcctctagcaccgtggtatgggaacaagacgcagatccat | |
| | tcgcagggaccgcagacgatttccccgcttttaacgaggaggagcttgct | |
| | tggctcatggagctgctgcctcagggtggatccgggggacttctcgaccc | |
| | aggcacacccatggatgccgatctcgtcgccagttctaccgttgtctggg | |
| | agcaagacgcagaccctttcgccggaactgcagacgacttccctgccttt | |
| | aatgaagaagaactggcttggctcatggaactgctcccccaggctagagg | |
| | gggtagcggcgggggcggcagcggaggtgatgcacttgacgacttcgatc | |
| | ttgatatgcttggcagcgacgcacttgatgacttcgatttggatatgctg | |
| | gggtccgatgctcttgacgattttgatttggacatgttgggttcagacgc | |
| | actcgatgatttcgatttggacatgctggcacgtggaagcgatgcccttg | |
| | atgactttgacctggatatgctcgggtccgatgctttggacgacttcgac | |
| | ctcgacatgttgggaagtgatgctcttgatgatttcgatcttgatatgct | |
| | tgggtccgacgctctcgatgattttgacctcgatatgctctgagcttgtc | |
| | aagcagatcgttcaaacatttggcaataaagtttcttaagattgaatcct | |
| | gttgccggtcttgcgatgattatcatataatttctgttgaattacgttaa | |
| | gcatgtaataattaacatgtaatgcatgacgttatttatgagatgggttt | |
| | ttatgattagagtcccgcaattatacatttaatacgcgatagaaaacaaa | |
| | atatagcgcgcaaactaggataaattatcgcgcgcggtgtcatctatgtt | |
| | actagatcgacgctttacgaattcccatggggaggacgtcgttgtggttg | |
| | gtgctttccttacattctgagcctctttccttctaatccactcatctgca | |
| | tcttcttgtgtccttactaatacctcattggttccaaattccctcccttt | |
| | aagcaccagctcgtttctgttcttccacagcctcccaagtatccaaggga | |
| | ctaaagcctccacattcttcagatcaggatattcttgtttaagatgttga | |
| | actctatggaggtttgtatgaactgatgatctaggaccggataagttccc | |
| | ttcttcatagcgaacttattcaaagaatgttttgtgtatcattcttgtta | |
| | cattgttattaatgaaaaaatattattggtcattggactgaacacgagtg | |
| | ttaaatatggaccaggccccaaataagatccattgatatatgaattaaat | |
| | aacaagaataaatcgagtcaccaaaccacttgccttttttaacgagactt | |
| | gttcaccaacttgatacaaaagtcattatcctatgcaaatcaataatcat | |
| | acaaaaatatccaataacactaaaaaaattaaaagaaatggataatttca | |
| | caatatgttatacgataaagaagttacttttccaagaaattcactgattt | |
| | tataagcccacttgcattagataaatggcaaaaaaaaacaaaaaggaaaa | |
| | gaaataaagcacgaagaattctagaaaatacgaaatacgcttcaatgcag | |
| | tgggacccacggttcaattattgccaattttcagctccaccgtatattta | |
| | aaaaataaaacgataatgctaaaaaaatataaatcgtaacgatcgttaaa | |
| | tctcaacggctggatcttatgacgaccgttaaggaaattgtggttgtcgg | |
| | acgaagtccagtaataaacggcgtcaaagtggttgcagccggcacacacg | |
| | agtcgtgtttatcaactcaaagcacaaatacttttcctcaacctaaaaat | |
| | aaggcaattagccaaaaacaactttgcgtgtaaacaacgctcaatacacg | |
| | tgtcattttattattagctattgcttcaccgccttagctttctcgtgacc | |
| | tagtcgtcctcgtcttttcttcttcttcttctataaaacaatacccaaag | |
| | agctcttcttcttcacaattcagatttcaatttctcaaaatcttaaaaac | |
| | tttctctcaattctctctaccgtgatcaaggtaaatttctgtgttcctta | |
| | ttctctcaaaatcttcgattttgttttcgttcgatcccaatttcgtatat | |
| | gttctttggtttagattctgttaatcttagatcgtagacgattttctggg | |
| | tttgatcgttagatatcatcttaattctcgattagggtttcatagatatc | |
| | atccgatttgttcaaataatttgagttttgtcgaataattactcttcgat | |
| | ttgtgatttctatctagatctggtgttagtttctagtttgtgcgatcgaa | |
| | tttgtcgattaatctgagtttttctgattaacagatgcagatcttaatga | |
| | caaagcaccagtggtctagtggtagaatagtaccctgccacggtacagac | |
| | ccgggttcgattcccggctggtgcattttgtatctctctatatctgtttt | |
| | agagctagaaatagcaagttaaaataaggctagtccgttatcaacttgaa | |
| | aaagtggcaccgagtcggtgcaacaaagcaccagtggtctagtggtagaa | |
| | tagtaccctgccacggtacagacccgggttcgattcccggctggtgcatt | |
| | tctttgtgtctcagctcagttttagagctagaaatagcaagttaaaataa | |
| | ggctagtccgttatcaacttgaaaaagtggcaccgagtcggtgcaacaaa | |
| | gcaccagtggtctagtggtagaatagtaccctgccacggtacagacccgg | |
| | gttcgattcccggctggtgcatgggaaaagattcagaactcgttttagag | |
| | ctagaaatagcaagttaaaataaggctagtccgttatcaacttgaaaaag | |
| | tggcaccgagtcggtgccaacaaagcaccagtggtctagtggtagaatag | |
| | taccctgccacggtacagacccgggttcgattcccggctggtgcagcttc | |
| | tctagctagagtcgatcgacaagctcgagtttctccataataatgtgtga | |
| | gtagttcccagataagggaattagggttcctatagggtttcgctcatgtg | |
| | ttgagcatataagaaacccttagtatgtatttgtatttgtaaaatacttc | |
| | tatcaataaaatttctaattcctaaaaccaaaatccagtactaaaatcca | |
| | gatcgctcagagaggatgcacatgtgaccgagggacacgaagtgatccgt | |
| | ttaaactatcagtgtttgacaggatatattggcgggtaaacctaagagaa | |
| | aagagcgtttattagaataatcggatatttaaaagggcgtgaaaaggttt | |
| | atccgttcgtccatttgtatgtgccagccgcctttgcgacgctcaccggg | |
| | ctggttgccctcgccgctgggctggcggccgtctatggccctgcaaacgc | |
| | gccagaaacgccgtcgaagccgtgtgcgagacaccgcggccgccggcgtt | |
| | gtggatacctcgcggaaaacttggccctcactgacagatgaggggcggac | |
| | gttgacacttgaggggccgactcacccggcgcggcgttgacagatgaggg | |
| | gcaggctcgatttcggccggcgacgtggagctggccagcctcgcaaatcg | |
| | gcgaaaacgcctgattttacgcgagtttcccacagatgatgtggacaagc | |
| | ctggggataagtgccctgcggtattgacacttgaggggcgcgactactga | |
| | cagatgaggggcgcgatccttgacacttgaggggcagagtgctgacagat | |
| | gaggggcgcacctattgacatttgaggggctgtccacaggcagaaaatcc | |
| | agcatttgcaagggtttccgcccgtttttcggccaccgctaacctgtctt | |
| | ttaacctgcttttaaaccaatatttataaaccttgtttttaaccagggct | |
| | gcgccctgtgcgcgtgaccgcgcacgccgaaggggggtgcccccccttct | |
| | cgaaccctcccggcccgctaacgcgggcctcccatccccccaggggctgc | |
| | gcccctcggccgcgaacggcctcaccccaaaaatggcagcgctggccaat | |
| | tcccgagtgcgcggaacccctatttgtttatttttctaaatacattcaaa | |
| | tatgtatccgctcatgagacaataaccctgataaatgcttcaataatatt | |
| | gaaaaaggaagagtatggctaaaatgagaatatcaccggaattgaaaaaa | |
| | ctgatcgaaaaataccgctgcgtaaaagatacggaaggaatgtctcctgc | |
| | taaggtatataagctggtgggagaaaatgaaaacctatatttaaaaatga | |
| | cggacagccggtataaagggaccacctatgatgtggaacgggaaaaggac | |
| | atgatgctatggctggaaggaaagctgcctgttccaaaggtcctgcactt | |
| | tgaacggcatgatggctggagcaatctgctcatgagtgaggccgatggcg | |
| | tcctttgctcggaagagtatgaagatgaacaaagccctgaaaagattatc | |
| | gagctgtatgcggagtgcatcaggctctttcactccatcgacatatcgga | |
| | ttgtccctatacgaatagcttagacagccgcttagccgaattggattact | |
| | tactgaataacgatctggccgatgtggattgcgaaaactgggaagaggac | |
| | actccatttaaagatccgcgcgagctgtatgattttttaaagacggaaaa | |
| | gcccgaagaggaacttgtcttttcccacggcgacctgggagacagcaaca | |
| | tctttgtgaaagatggcaaagtaagtggctttattgatcttgggagaagc | |
| | ggcagggcggacaagtggtatgacattgccttctgcgtccggtcgctcag | |
| | ggaggatatcggggaagaacagtatgtcgagctattttttgacttactgg | |
| | ggatcaagcctgattgggagaaaataaaatattatattttactggatgaa | |
| | ttgttttagctgtcagaccaagtttactcatatatactttagattgattt | |
| | aaaacttcatttttaatttaaaaggatctaggtgaagatcctttttgata | |
| | atctcatgaccaaaatcccttaacgtgagttttcgttccactgagcgtca | |
| | gaccccgtagaaaagatcaaaggatcttcttgagatcctttttttctgcg | |
| | cgtaatctgctgcttgcaaacaaaaaaaccaccgctaccagcggtggttt | |
| | gtttgccggatcaagagctaccaactctttttccgaaggtaactggcttc | |
| | agcagagcgcagataccaaatactgtccttctagtgtagccgtagttagg | |
| | ccaccacttcaagaactctgtagcaccgcctacatacctcgctctgctaa | |
| | tcctgttaccagtggctgctgccagtggcgataagtcgtgtcttaccggg | |
| | ttggactcaagacgatagttaccggataaggcgcagcggtcgggctgaac | |
| | ggggggttcgtgcacacagcccagcttggagcgaacgacctacaccgaac | |
| | tgagatacctacagcgtgagctatgagaaagcgccacgcttcccgaaggg | |
| | agaaaggcggacaggtatccggtaagcggcagggtcggaacaggagagcg | |
| | cacgagggagcttccagggggaaacgcctggtatctttatagtcctgtcg | |
| | ggtttcgccacctctgacttgagcgtcgatttttgtgatgctcgtcaggg | |
| | gggggagcctatggaaaaacgccagcaacgcggcctttttacggttcctg | |
| | gcagatcctagatgtggcgcaacgatgccggcgacaagcaggagcgcacc | |
| | gacttcttccgcatcaagtgttttggctctcaggccgaggcccacggcaa | |
| | gtatttgggcaaggggtcgctggtattcgtgcagggcaagattcggaata | |
| | ccaagtacgagaaggacggccagacggtctacgggaccgacttcattgcc | |
| | gataaggtggattatctggacaccaaggcaccagggggtcaaatcaggaa | |
| | taagggcacattgccccggcgtgagtcggggcaatcccgcaaggagggtg | |
| | aatgaatcggacgtttgaccggaaggcatacaggcaagaactgatcgacg | |
| | cggggttttccgccgaggatgccgaaaccatcgcaagccgcaccgtcatg | |
| | cgtgegccccgcgaaaccttccagtccgtcggctcgatggtccagcaagc | |
| | tacggccaagategagcgcgacagcgtgcaactggctccccctgccctgc | |
| | ccgcgccateggccgccgtggagcgttcgcgtcgtcttgaacaggaggcg | |
| | gcaggtttggcgaagtcgatgaccatcgacacgcgaggaactatgacgac | |
| | caagaagcgaaaaaccgccggcgaggacctggcaaaacaggtcagcgagg | |
| | ccaagcaggccgcgttgctgaaacacacgaagcagcagatcaaggaaatg | |
| | cagctttccttgttcgatattgcgccgtggccggacacgatgcgagcgat | |
| | gccaaacgacacggcccgctctgccctgttcaccacgcgcaacaagaaaa | |
| | tcccgcgcgaggcgctgcaaaacaaggtcattttccacgtcaacaaggac | |
| | gtgaagatcacctacaccggcgtcgagctgcgggccgacgatgacgaact | |
| | ggtgtggcagcaggtgttggagtacgcgaagcgcacccctatcggcgagc | |
| | cgatcaccttcacgttctacgagctttgccaggacctgggctggtcgatc | |
| | aatggccggtattacacgaaggccgaggaatgcctgtcgcgcctacaggc | |
| | gacggcgatgggcttcacgtccgaccgcgttgggcacctggaatcggtgt | |
| | cgctgctgcaccgcttccgcgtcctggaccgtggcaagaaaacgtcccgt | |
| | tgccaggtcctgatcgacgaggaaatcgtcgtgctgtttgctggcgacca | |
| | ctacacgaaattcatatgggagaagtaccgcaagctgtcgccgacggccc | |
| | gacggatgttcgactatttcagctcgcaccgggagccgtacccgctcaag | |
| | ctggaaaccttccgcctcatgtgcggatcggattccacccgcgtgaagaa | |
| | gtggcgcgagcaggtcggcgaagcctgcgaagagttgcgaggcagcggcc | |
| | tggtggaacacgcctgggtcaatgatgacctggtgcattgcaaacgctag | |
| | ggccttgtggggtcagttccggctgggggttcagcagcccctgctcggat | |
| | ctgttggaccggacagtagtcatggttgatgggctgcctgtatcgagtgg | |
| | tgattttgtgccgagctgccggtcggggagctgttggctggctggtggca | |
| | ggatatattgtggtgtaaacaaattgacgcttagacaacttaataacaca | |
| | ttgcggacgtttttaatgtactggggttgaacactct | |
| 107 | gtgccgaattcggatccagcgatctggattttagtactggattttggttt | RC4261 |
| | taggaattagaaattttattgatagaagtattttacaaatacaaatacat | M. Frank |
| | actaagggtttcttatatgctcaacacatgagcgaaaccctataggaacc | AINTEGU |
| | ctaattcccttatctgggaactactcacacattattatggagaaactcga | MENTA-A |
| | gcttgtcgatcgactctagctagagaagctcactttccaacaaccttagt | dCas9-TV |
| | aatttctcccttaacataatgaacaaattcttcaagttgatcagctctag | Construct |
| | aagcaagtctatcttcttcttgtccaagataagcttgtctagcttcaaga | (RCGG29 |
| | ataactggttgatattgagctggaagtctttccatagcccaatcagcagc | 94) |
| | aacatcctttggagcaatctttccagtaacagcagaataccaaattctag | |
| | aaagagtaagaacaacatttctttcatctccagcccaatctggtggagaa | |
| | ttccaaagagtaagagtttcattaagagcttcaaaaagatcttgttctgg | |
| | aactggatcaaaaagttcttcagcagctggtccaacaagagcaacagaat | |
| | gttctctagccttagtaagaagaatagcaagatcaatatcaatagtagct | |
| | ggttcaaaaattccagcaagaatatcatttctttgccattctccaaattg | |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtgtgctccaccatgttatcac | |
| | atcaatccacttgctttgtagacgtggttggaacctcttctttttccacg | |
| | atgctcctcgtggggggggtccatctttgggaccactgtcggcagagaga | |
| | tcttgaatgatagcctttcctttatcgcaatgatggcatttgtaggagcc | |
| | accttccttttctactgtcctttcgatgaagtgacagatagctgggcaat | |
| | ggaatccgaggaggtttcccgaaattatcctttgttgaaaagtctcaata | |
| | gccctttgatcttctgagactgtatctttgacatttttggagtagaccag | |
| | agtgtcgtgctccaccatgttgacctccgcaagaattcaagcttagcgtt | |
| | cctaacacctggagaaccttttatgtacttcacaaccctcaatgctgctt | |
| | ccaggtgtgactgtttgggttgctgcatgaattgactcagcacctgcact | |
| | gcaaagctgatatctggccttgtgattgtcaagtagagaagctttcccac | |
| | cagtctctgataggatcccacatcctctaactctgcatcatcagtctttc | |
| | ctaagtgcttgtcatattctacagtagtcagcctttggttctgttccatt | |
| | ggggttgacactggtctgcagcctcccagaccaacacctgatatcagttc | |
| | cagtgcatacttcctctggttcagtaggattcccttttctgatctcagca | |
| | cctcaatgcctaagaagtattttagttctcccaaatctttcatcttgaaa | |
| | tgctgatgcagggttgcctttgcttctgaaatcaaaacattgctgctgcc | |
| | tgttattaacagatcatccacataaatcaggattatgacaaggtcagtcc | |
| | cctctcttttggtaaacaaggagtgatcataagcactttgcataaaacca | |
| | gcctgcataaggacagtggtaagcttgatgttccactgccttgatgcttg | |
| | ctttaaaccatagagggattcaacagcctgcacactttggtttccccttg | |
| | gctgtgaaaaccctgaggcagagacatataaacttcttccatgaggtcac | |
| | cttgtagaaaagcattgttgacatccatctggaaaaggaaccagcccttg | |
| | gaagcagcaacagatatgacagcttttacagtgaccattttggccactgg | |
| | agaaaaagtttcatggtagtcaaggccttcttgctgagtgtatcccttgg | |
| | ccactagccttgccttaaacctgtcaacttcaccattagctttgtattta | |
| | attttgtacacccatttggaccctataggctgtttaccagggggtaaagg | |
| | gacaatctcccaggtgttattatcctcaagagcctgtatctcaagggaca | |
| | tggcctccatccatttctcatcttgagctgcttctttgaaggatttaggt | |
| | tcagtataagtggagaaagcactcaaataagcttgatagtgaacaggcaa | |
| | gtgatcataggaaacatagttggctatagggtatggaacatccctagagc | |
| | ctttgttcagtgtcacaaagtccttgagccagatgggaggacctgcattg | |
| | cgtttaggtctagtgtgcaggtttgctggaactagagagggatcagctac | |
| | agcagtatggtgctcaaattcagcatttgctaggtcaggctcagctgact | |
| | caactgactctgcaggagcatgcaggtggcctgaaggtgcagcatcagct | |
| | gaagtgatatgattttgggtagaatgagtccccaaagtgatatcctcatt | |
| | ggcctctacaatgtcaggaataagaactggagtatcatcatcatcagtat | |
| | gaaagctggaaggaaaaatatcattatacactagctgcaaagctgtatct | |
| | tcagagctaagtgaacctgctcgagtgaacatatcaggttcatgggagat | |
| | ggactctttgaaagggaactgaaactctctgaaaactacatccctgctca | |
| | ctatgatcaccttattatccaagtcatacaacctgtaacccttttgagtc | |
| | tcagaataaccaatgaagatggttcgcttggctctaggtgcaagtttgtc | |
| | acctttgggcagtgttgctgcaaaagcaagacacccaaacactctcaagt | |
| | gatcaagcttggcatgtttctgatagagtagttcatatggacatttgcct | |
| | tgtaagattggagtagggagtctattgatcaggtatacaacagtcttgac | |
| | acagtctccccaaaacctggtaggtacaccactctgaaacttaagtgccc | |
| | ttgccatctcaaggatgtgtctgtgctttctctccacaacaccattctgt | |
| | tgtggtgtgtagggacagctactttgatgaacaatcccaagagaggccag | |
| | caactcattacaacttctccactagaattcgagctcggagtgttcaaaaa | |
| | taaaaagtgattttgagatataatatacatacgacaattaatacaatatt | |
| | atttttgtgtctatccctctcttagaccctattatttatcacatttgaaa | |
| | ttcagaatttttttttctctcgttgttgtacaaatatctaatcgttaatt | |
| | attattaatgcattaaatatttaaaatataattaatgatattaattttcc | |
| | tagcgtaagactaataataaatatcgacattatgcgagattatgacatga | |
| | tgttatgttaatagtacagaataaagtaaaaaaatatatataacaataat | |
| | aatttaaaatatttaaaatgacatataaagtagttttctaaaacttcagt | |
| | gattagttcaatttattatttcgcaacaattatgatggttaattaaggtg | |
| | aattagaagaagaactgaaaattaagataattaaggagaacaaatgagca | |
| | cttcaaaatttgctattaagccgactctgctagccatgcatgttggatgt | |
| | aacttcacccgattgatgggtccttaattataatttttcttatgataaaa | |
| | aataaattgctactaaggcgtactgaactattcgaaatgacaacactaca | |
| | aggcctcgacaacgttaacttttttccaatttagtggggcgttacaagct | |
| | aaagcttttttatttttgaactttaaattgctctttcactttcatttaat | |
| | aacgcctaaacctagctacgagcaatataacctgggtgtatatagctata | |
| | tatatcatatgcagagcaatctgtaaccccaaggataaaacgttgtgttg | |
| | cttattatagaaatcacagttttaaatggaacgagtgacagagtttgttt | |
| | gtttctaaataggataggaggcatttaatcggaaattggacaggagaaag | |
| | ttgttttctttctgcaaattatgcctccagaacttgttttacggtaagaa | |
| | aatttacaccaaatttacaagtattggttctgaacgctacttgcggaaat | |
| | tttatgccgccagtgacttcttggcactttaaataagttaattagttaat | |
| | catttgaaaatatattaacaagaagaatttttttctagtgtaatcaagag | |
| | gtattttaagcttctatcttacactcaattaatcttctattctcctattg | |
| | attggtctattttaaagtaaagtaatatattcaattacgacttcatgcta | |
| | aattcaaagattataacagtaaaaacaatttttattcacctctattcaaa | |
| | ctgccagtttagcgcaagtgtttatttttctgataaacttgcggccgcac | |
| | aaaacccagcattgcttcaatgtgcataagcatgggtacggtggtggtgt | |
| | ttctccaacctccatttcagattgtcttgatttgtaattcaaacacacac | |
| | ttagtcttaaaatcatattttctcatatctcaattgtgatttttccaaat | |
| | gcgttgtgtgtaggcgaagctaagtttaggttattgctcaagtgccacaa | |
| | gtttgagagggttttactgtcttttttatttaatctagaaatatgatttt | |
| | tggacacgtctacttgttacattccgtttacttatggtagtatttgacta | |
| | ataaagacattaagattatagtagcatttgactaataaagacacgcgtac | |
| | gtaggtcactattttattggctaagtaaaaaaaaaaaaaaacacgaaaaa | |
| | actttcaaacaaaggggaagcggagaaatcagcctaacttgatattagct | |
| | gtacaaaatacataaatagctgtcggaaataattatctcttgcagtatat | |
| | atatagaaagtagcagactctaaaatataacatgaagatgtgattaagca | |
| | tttattccattatatggcccataaacatgcatgatgcaaatagaaggatg | |
| | tagggtaaagttgccctaagaaacgaattgaactgcggtcgttgcggtat | |
| | gagaaagagagagagaaaagtgagtgaaggaaggtgggagaatggtttcc | |
| | gtgacggagtaggacggaaagtaagggtccccacagtgcacatgggagcg | |
| | gatttgacgatgagtcgagaaagagcacaagacgtttatgacaaagcctg | |
| | caatagctttcgttaatttggcaaaagacccctaccatttgcggcaatgc | |
| | acctccccgttctattatttctctttccctcattcattatatcccctatt | |
| | ctctgccccagatcgatcgaacaagtgaattagctagaagcattgaagga | |
| | cgtagctgctccatggattacaaagatgacgatgataaagactacaagga | |
| | cgacgacgataaaatggcccctaaaaagaagcgcaaggtgggtattcatg | |
| | gggtgcctgcagctggaatggataagaagtactccatcggactggctatc | |
| | ggaactaactccgtgggatgggctgtgatcactgatgagtacaaggtgcc | |
| | atccaagaagttcaaggtgctgggaaacactgatagacactccatcaaga | |
| | agaacctgatcggagctctgctgttcgattccggagagactgctgaggct | |
| | actagactgaagagaactgctagaagaagatacactagaagaaagaacag | |
| | aatctgctacctgcaagagatcttctccaacgagatggctaaggtggatg | |
| | attccttcttccacagactggaggagtccttcctggtggaggaggataag | |
| | aagcacgagagacacccaatcttcggaaacatcgtggatgaggtggctta | |
| | ccacgagaagtacccaactatctaccacctgagaaagaagctggtggatt | |
| | ccactgataaggctgatctgagactgatctacctggctctggctcacatg | |
| | atcaagttcagaggacacttcctgatcgagggagatctgaacccagataa | |
| | ctccgatgtggataagctgttcatccaactggtgcaaacttacaaccaac | |
| | tgttcgaggagaacccaatcaacgcttccggagtggatgctaaggctatc | |
| | ctgtccgctagactgtccaagtccaggagactggagaacctgatcgctca | |
| | actgccaggagagaagaagaacggactgttcggaaacctgatcgctctgt | |
| | ccctgggactgactccaaacttcaagtccaacttcgatctggctgaggat | |
| | gctaagctgcaactgtccaaggatacttacgatgatgatctggataacct | |
| | gctggctcaaatcggagatcaatacgctgatctgttcctggctgctaaga | |
| | acctgtccgatgctatcctgctgtccgatatcctgagagtgaacactgag | |
| | atcactaaggctccactgtccgcttccatgatcaagagatacgatgagca | |
| | ccaccaagatctgactctgctgaaggctctggtgagacaacaactgccag | |
| | agaagtacaaggagatcttcttcgatcaatccaagaacggatacgctgga | |
| | tacatcgatggaggagcttcccaagaggagttctacaagttcatcaagcc | |
| | aatcctggagaagatggatggaactgaggagctgctggtgaagctgaaca | |
| | gagaggatctgctgagaaagcaaagaactttcgataacggatccatccca | |
| | caccaaatccacctgggagagctgcacgctatcctgaggagacaagagga | |
| | tttctacccattcctgaaggataacagagagaagatcgagaagatcctga | |
| | ctttcagaatcccatactacgtgggaccactggctagaggaaactccaga | |
| | ttcgcttggatgactagaaagtccgaggagactatcactccatggaactt | |
| | cgaggaggtaagtttctgcttctacctttgatatatatataataattatc | |
| | attaattagtagtaatataatatttcaaatatttttttcaaaataaaaga | |
| | atgtagtatatagcaattgcttttctgtagtttataagtgtgtatatttt | |
| | aatttataacttttctaatatatgaccaaaatttgttgatgtgcaggtgg | |
| | tggataagggagcttccgctcaatccttcatcgagagaatgactaacttc | |
| | gataagaacctgccaaacgagaaggtgctgccaaagcactccctgctgta | |
| | cgagtacttcactgtgtacaacgagctgactaaggtgaagtacgtgactg | |
| | agggaatgagaaagccagctttcctgtccggagagcaaaagaaggctatc | |
| | gtggatctgctgttcaagactaacagaaaggtgactgtgaagcaactgaa | |
| | ggaggattacttcaagaagatcgagtgcttcgattccgtggagatctccg | |
| | gagtggaggatagattcaacgcttccctgggaacttaccacgatctgctg | |
| | aagatcatcaaggataaggatttcctggataacgaggagaacgaggatat | |
| | cctggaggatatcgtgctgactctgactctgttcgaggatagagagatga | |
| | tcgaggagagacttaagacttacgctcacctgttcgatgataaggtgatg | |
| | aagcaactgaagagaagaagatacactggatggggtagactgtccagaaa | |
| | gctgatcaacggaatcagagataagcaatccggaaagactatcctggatt | |
| | tcctgaagtccgatggattcgctaacagaaacttcatgcaactgatccac | |
| | gatgattccctgactttcaaggaggatatccaaaaggctcaagtgtccgg | |
| | acaaggagattccctgcacgagcacatcgctaacctggctggatccccag | |
| | ctatcaagaagggaatcctgcaaactgtgaaggtggtggatgagctggtg | |
| | aaggtgatgggtagacacaagccagagaacatcgtgatcgagatggctag | |
| | agagaaccaaactactcaaaagggacaaaagaactccagagagagaatga | |
| | agagaatcgaggagggaatcaaggagctgggatcccaaatcctgaaggag | |
| | cacccagtggagaacactcaactgcaaaacgagaagctgtacctgtacta | |
| | cctgcaaaacggaagagatatgtacgtggatcaagagctggatatcaaca | |
| | gactgtccgattacgatgtggatgcaatcgtgccacaatccttcctgaag | |
| | gatgattccatcgataacaaggtgctgactagatccgataagaacagagg | |
| | aaagtccgataacgtgccatccgaggaggtggtgaagaagatgaagaact | |
| | actggagacaactgctgaacgctaagctgatcactcaaagaaagttcgat | |
| | aacctgactaaggctgagagaggaggactgtccgagctggataaggctgg | |
| | attcatcaagagacaactggtggagactagacaaatcactaagcacgtgg | |
| | ctcaaatcctggattccagaatgaacactaagtacgatgagaacgataag | |
| | ctgatcagagaggtgaaggtgatcactctgaagtccaagctggtgtccga | |
| | tttcagaaaggatttccaattctacaaggtgagagagatcaacaactacc | |
| | accacgctcacgatgcttacctgaacgctgtggtgggaactgctctgatc | |
| | aagaagtacccaaagctggagtccgagttcgtgtacggagattacaaggt | |
| | gtacgatgtgagaaagatgatcgctaagtccgagcaagagatcggaaagg | |
| | ctactgctaagtacttcttctactccaacatcatgaacttcttcaagact | |
| | gagatcactctggctaacggagagatcagaaaaagaccactgatcgagac | |
| | taacggagagactggagagatcgtgtgggataagggaagagatttcgcta | |
| | ctgtgagaaaggtgctgtccatgccacaagtgaacatcgtgaaaaagact | |
| | gaggtgcaaactggaggattctccaaggagtccatcctgccaaagagaaa | |
| | ctccgataagctgatcgctagaaagaaggattgggatccaaagaagtacg | |
| | gaggattcgattccccaactgtggcttactccgtgctggtggtggctaag | |
| | gtggagaagggaaagtccaagaagctgaagtccgtgaaggagctgctggg | |
| | aatcactatcatggagagatcctccttcgagaagaacccaatcgatttcc | |
| | tggaggctaagggatacaaggaggtgaagaaggatctgatcatcaagctg | |
| | ccaaagtactccctgttcgagctggagaacggaagaaagagaatgctggc | |
| | ttccgctggagagctgcaaaagggaaacgagctggctctgccatccaagt | |
| | acgtgaacttcctgtacctggcttcccactacgagaagctgaagggatcc | |
| | ccagaggataacgagcaaaagcaactgttcgtggagcaacacaagcacta | |
| | cctggatgagatcatcgagcaaatctccgagttctccaagagagtgatcc | |
| | tggctgatgctaacctggataaggtgctgtccgcttacaacaagcacaga | |
| | gataagccaatcagagagcaagctgagaacatcatccacctgttcactct | |
| | gactaacctgggagctccagctgctttcaagtacttcgatactactatcg | |
| | atagaaagagatacacttccactaaggaggtgctggatgctactctgatc | |
| | caccaatccatcactggactgtacgagactagaatcgatctgtcccaact | |
| | gggaggagatagttcgaagaggcctgccgccacaaagaaagccggccagg | |
| | caaagaaaaaaaaacccaggggagggagtggtggactectcgaccctggc | |
| | actccaatggacgccgatctcgtagcatcatctaccgtcgtgtgggagca | |
| | ggacgccgacccttttgctggcacagctgatgatttcccagcttttaatg | |
| | aggaagaattggcttggctgatggagcttctcccacaaggtggtagcggc | |
| | gggctcctcgatcccggtactcccatggacgcagatcttgttgctagctc | |
| | cactgttgtatgggagcaggacgccgatcccttcgctggaaccgccgatg | |
| | atttcccagcattcaacgaggaagaacttgcatggctcatggagctcttg | |
| | ccccaagctcgcggtggatctgggggtttgttggatcccggaaccccaat | |
| | ggacgcagacctcgtggcttctagtaccgtagtatgggaacaggacgctg | |
| | accctttcgccggcaccgcagatgacttcccagcattcaacgaggaggaa | |
| | ctggcctggctgatggaacttctgcctcagggtggatccggaggcctctt | |
| | ggaccccggtacccctatggacgctgacctggtcgcatcttctaccgtgg | |
| | tttgggagcaagatgccgacccattcgcaggaacagctgatgacttcccc | |
| | gcattcaatgaagaagagctcgcatggctgatggaattgcttccacaagc | |
| | cagaggcggctctggagggcttcttgatcccggaacacccatggatgccg | |
| | acctcgtggcctctagcaccgtggtatgggaacaagacgcagatccattc | |
| | gcagggaccgcagacgatttccccgcttttaacgaggaggagcttgcttg | |
| | gctcatggagctgctgcctcagggtggatccgggggacttctcgacccag | |
| | gcacacccatggatgccgatctcgtcgccagttctaccgttgtctgggag | |
| | caagacgcagaccctttcgccggaactgcagacgacttccctgcctttaa | |
| | tgaagaagaactggcttggctcatggaactgctcccccaggctagagggg | |
| | gtagcggcgggggcggcagcggaggtgatgcacttgacgacttcgatctt | |
| | gatatgcttggcagcgacgcacttgatgacttcgatttggatatgctggg | |
| | gtccgatgctcttgacgattttgatttggacatgttgggttcagacgcac | |
| | tcgatgatttcgatttggacatgctggcacgtggaagcgatgcccttgat | |
| | gactttgacctggatatgctcgggtccgatgctttggacgacttcgacct | |
| | cgacatgttgggaagtgatgctcttgatgatttcgatcttgatatgcttg | |
| | ggtccgacgctctcgatgattttgacctcgatatgctctgagcttgtcaa | |
| | gcagatcgttcaaacatttggcaataaagtttcttaagattgaatcctgt | |
| | tgccggtcttgcgatgattatcatataatttctgttgaattacgttaagc | |
| | atgtaataattaacatgtaatgcatgacgttatttatgagatgggttttt | |
| | atgattagagtcccgcaattatacatttaatacgcgatagaaaacaaaat | |
| | atagcgcgcaaactaggataaattatcgcgcgcggtgtcatctatgttac | |
| | tagatcgacgctttacgaattcccatggggaggacgtcgttgtggttggt | |
| | gctttccttacattctgagcctctttccttctaatccactcatctgcatc | |
| | ttcttgtgtccttactaatacctcattggttccaaattccctccctttaa | |
| | gcaccagctcgtttctgttcttccacagcctcccaagtatccaagggact | |
| | aaagcctccacattcttcagatcaggatattcttgtttaagatgttgaac | |
| | tctatggaggtttgtatgaactgatgatctaggaccggataagttccctt | |
| | cttcatagcgaacttattcaaagaatgttttgtgtatcattcttgttaca | |
| | ttgttattaatgaaaaaatattattggtcattggactgaacacgagtgtt | |
| | aaatatggaccaggccccaaataagatccattgatatatgaattaaataa | |
| | caagaataaatcgagtcaccaaaccacttgccttttttaacgagacttgt | |
| | tcaccaacttgatacaaaagtcattatcctatgcaaatcaataatcatac | |
| | aaaaatatccaataacactaaaaaaattaaaagaaatggataatttcaca | |
| | atatgttatacgataaagaagttacttttccaagaaattcactgatttta | |
| | taagcccacttgcattagataaatggcaaaaaaaaacaaaaaggaaaaga | |
| | aataaagcacgaagaattctagaaaatacgaaatacgcttcaatgcagtg | |
| | ggacccacggttcaattattgccaattttcagctccaccgtatatttaaa | |
| | aaataaaacgataatgctaaaaaaatataaatcgtaacgatcgttaaatc | |
| | tcaacggctggatcttatgacgaccgttaaggaaattgtggttgtcggac | |
| | gaagtccagtaataaacggcgtcaaagtggttgcagccggcacacacgag | |
| | tcgtgtttatcaactcaaagcacaaatacttttcctcaacctaaaaataa | |
| | ggcaattagccaaaaacaactttgcgtgtaaacaacgctcaatacacgtg | |
| | tcattttattattagctattgcttcaccgccttagctttctcgtgaccta | |
| | gtcgtcctcgtcttttcttcttcttcttctataaaacaatacccaaagag | |
| | ctcttcttcttcacaattcagatttcaatttctcaaaatcttaaaaactt | |
| | tctctcaattctctctaccgtgatcaaggtaaatttctgtgttccttatt | |
| | ctctcaaaatcttcgattttgttttcgttcgatcccaatttcgtatatgt | |
| | tctttggtttagattctgttaatcttagatcgtagacgattttctgggtt | |
| | tgatcgttagatatcatcttaattctcgattagggtttcatagatatcat | |
| | ccgatttgttcaaataatttgagttttgtcgaataattactcttcgattt | |
| | gtgatttctatctagatctggtgttagtttctagtttgtgcgatcgaatt | |
| | tgtcgattaatctgagtttttctgattaacagatgcagatcttaatgaca | |
| | aagcaccagtggtctagtggtagaatagtaccctgccacggtacagaccc | |
| | gggttcgattcccggctggtgcaaaaaaatgtgaatgtggatggttttag | |
| | agctagaaatagcaagttaaaataaggctagtccgttatcaacttgaaaa | |
| | agtggcaccgagtcggtgcaacaaagcaccagtggtctagtggtagaata | |
| | gtaccctgccacggtacagacccgggttcgattcccggctggtgcaaaat | |
| | ttataacatttacatcgttttagagctagaaatagcaagttaaaataagg | |
| | ctagtccgttatcaacttgaaaaagtggcaccgagtcggtgcaacaaagc | |
| | accagtggtctagtggtagaatagtaccctgccacggtacagacccgggt | |
| | tcgattcccggctggtgcatgatgtctttagaccagacagttttagagct | |
| | agaaatagcaagttaaaataaggctagtccgttatcaacttgaaaaagtg | |
| | gcaccgagtcggtgccaacaaagcaccagtggtctagtggtagaatagta | |
| | ccctgccacggtacagacccgggttcgattcccggctggtgcagcttctc | |
| | tagctagagtcgatcgacaagctcgagtttctccataataatgtgtgagt | |
| | agttcccagataagggaattagggttcctatagggtttcgctcatgtgtt | |
| | gagcatataagaaacccttagtatgtatttgtatttgtaaaatacttcta | |
| | tcaataaaatttctaattcctaaaaccaaaatccagtactaaaatccaga | |
| | tcgctcagagaggatgcacatgtgaccgagggacacgaagtgatccgttt | |
| | aaactatcagtgtttgacaggatatattggcgggtaaacctaagagaaaa | |
| | gagcgtttattagaataatcggatatttaaaagggcgtgaaaaggtttat | |
| | ccgttcgtccatttgtatgtgccagccgcctttgcgacgctcaccgggct | |
| | ggttgccctcgccgctgggctggcggccgtctatggccctgcaaacgcgc | |
| | cagaaacgccgtcgaagccgtgtgcgagacaccgcggccgccggcgttgt | |
| | ggatacctcgcggaaaacttggccctcactgacagatgaggggcggacgt | |
| | tgacacttgaggggccgactcacccggcgcggcgttgacagatgaggggc | |
| | aggctcgatttcggccggcgacgtggagctggccagcctcgcaaatcggc | |
| | gaaaacgcctgattttacgcgagtttcccacagatgatgtggacaagcct | |
| | ggggataagtgccctgcggtattgacacttgaggggcgcgactactgaca | |
| | gatgaggggcgcgatccttgacacttgaggggcagagtgctgacagatga | |
| | ggggcgcacctattgacatttgaggggctgtccacaggcagaaaatccag | |
| | catttgcaagggtttccgcccgtttttcggccaccgctaacctgtctttt | |
| | aacctgcttttaaaccaatatttataaaccttgtttttaaccagggctgc | |
| | gccctgtgcgcgtgaccgcgcacgccgaaggggggtgcccccccttctcg | |
| | aaccctcccggcccgctaacgcgggcctcccatccccccaggggctgcgc | |
| | ccctcggccgcgaacggcctcaccccaaaaatggcagcgctggccaattc | |
| | ccgagtgcgcggaacccctatttgtttatttttctaaatacattcaaata | |
| | tgtatccgctcatgagacaataaccctgataaatgcttcaataatattga | |
| | aaaaggaagagtatggctaaaatgagaatatcaccggaattgaaaaaact | |
| | gatcgaaaaataccgctgcgtaaaagatacggaaggaatgtctcctgcta | |
| | aggtatataagctggtgggagaaaatgaaaacctatatttaaaaatgacg | |
| | gacagccggtataaagggaccacctatgatgtggaacgggaaaaggacat | |
| | gatgctatggctggaaggaaagctgcctgttccaaaggtcctgcactttg | |
| | aacggcatgatggctggagcaatctgctcatgagtgaggccgatggcgtc | |
| | ctttgctcggaagagtatgaagatgaacaaagccctgaaaagattatcga | |
| | gctgtatgcggagtgcatcaggctctttcactccatcgacatatcggatt | |
| | gtccctatacgaatagcttagacagccgcttagccgaattggattactta | |
| | ctgaataacgatctggccgatgtggattgcgaaaactgggaagaggacac | |
| | tccatttaaagatccgcgcgagctgtatgattttttaaagacggaaaagc | |
| | ccgaagaggaacttgtcttttcccacggcgacctgggagacagcaacatc | |
| | tttgtgaaagatggcaaagtaagtggctttattgatcttgggagaagcgg | |
| | cagggcggacaagtggtatgacattgccttctgcgtccggtcgctcaggg | |
| | aggatatcggggaagaacagtatgtcgagctattttttgacttactgggg | |
| | atcaagcctgattgggagaaaataaaatattatattttactggatgaatt | |
| | gttttagctgtcagaccaagtttactcatatatactttagattgatttaa | |
| | aacttcatttttaatttaaaaggatctaggtgaagatcctttttgataat | |
| | ctcatgaccaaaatcccttaacgtgagttttcgttccactgagcgtcaga | |
| | ccccgtagaaaagatcaaaggatcttcttgagatcctttttttctgcgcg | |
| | taatctgctgcttgcaaacaaaaaaaccaccgctaccagcggtggtttgt | |
| | ttgccggatcaagagctaccaactctttttccgaaggtaactggcttcag | |
| | cagagcgcagataccaaatactgtccttctagtgtagccgtagttaggcc | |
| | accacttcaagaactctgtagcaccgcctacatacctcgctctgctaatc | |
| | ctgttaccagtggctgctgccagtggcgataagtcgtgtcttaccgggtt | |
| | ggactcaagacgatagttaccggataaggcgcagcggtcgggctgaacgg | |
| | ggggttcgtgcacacagcccagcttggagcgaacgacctacaccgaactg | |
| | agatacctacagcgtgagctatgagaaagcgccacgcttcccgaagggag | |
| | aaaggcggacaggtatccggtaagcggcagggtcggaacaggagagcgca | |
| | cgagggagcttccagggggaaacgcctggtatctttatagtcctgtcggg | |
| | tttcgccacctctgacttgagcgtcgatttttgtgatgctcgtcaggggg | |
| | gcggagcctatggaaaaacgccagcaacgcggcctttttacggttcctgg | |
| | cagatcctagatgtggcgcaacgatgccggcgacaagcaggagcgcaccg | |
| | acttcttccgcatcaagtgttttggctctcaggccgaggcccacggcaag | |
| | tatttgggcaaggggtcgctggtattcgtgcagggcaagattcggaatac | |
| | caagtacgagaaggacggccagacggtctacgggaccgacttcattgccg | |
| | ataaggtggattatctggacaccaaggcaccagggggtcaaatcaggaat | |
| | aagggcacattgccccggcgtgagtcggggcaatcccgcaaggagggtga | |
| | atgaatcggacgtttgaccggaaggcatacaggcaagaactgatcgacgc | |
| | ggggttttccgccgaggatgccgaaaccatcgcaagccgcaccgtcatgc | |
| | gtgcgccccgcgaaaccttccagtccgtcggctcgatggtccagcaagct | |
| | acggccaagatcgagcgcgacagcgtgcaactggctccccctgccctgcc | |
| | cgcgccatcggccgccgtggagcgttcgcgtcgtcttgaacaggaggcgg | |
| | caggtttggcgaagtcgatgaccatcgacacgcgaggaactatgacgacc | |
| | aagaagcgaaaaaccgccggcgaggacctggcaaaacaggtcagcgaggc | |
| | caagcaggccgcgttgctgaaacacacgaagcagcagatcaaggaaatgc | |
| | agctttccttgttcgatattgcgccgtggccggacacgatgcgagcgatg | |
| | ccaaacgacacggcccgctctgccctgttcaccacgcgcaacaagaaaat | |
| | cccgcgcgaggcgctgcaaaacaaggtcattttccacgtcaacaaggacg | |
| | tgaagatcacctacaccggcgtcgagctgcgggccgacgatgacgaactg | |
| | gtgtggcagcaggtgttggagtacgcgaagcgcacccctatcggcgagcc | |
| | gatcaccttcacgttctacgagctttgccaggacctgggctggtcgatca | |
| | atggccggtattacacgaaggccgaggaatgcctgtcgcgcctacaggcg | |
| | acggcgatgggcttcacgtccgaccgcgttgggcacctggaatcggtgtc | |
| | gctgctgcaccgcttccgcgtcctggaccgtggcaagaaaacgtcccgtt | |
| | gccaggtcctgatcgacgaggaaatcgtcgtgctgtttgctggcgaccac | |
| | tacacgaaattcatatgggagaagtaccgcaagctgtcgccgacggcccg | |
| | acggatgttcgactatttcagctcgcaccgggagccgtacccgctcaagc | |
| | tggaaaccttccgcctcatgtgcggatcggattccacccgcgtgaagaag | |
| | tggcgcgagcaggtcggcgaagcctgcgaagagttgcgaggcagcggcct | |
| | ggtggaacacgcctgggtcaatgatgacctggtgcattgcaaacgctagg | |
| | gccttgtggggtcagttccggctgggggttcagcagcccctgctcggatc | |
| | tgttggaccggacagtagtcatggttgatgggctgcctgtatcgagtggt | |
| | gattttgtgccgagctgccggtcggggagctgttggctggctggtggcag | |
| | gatatattgtggtgtaaacaaattgacgcttagacaacttaataacacat | |
| | tgcggacgtttttaatgtactggggttgaacactct | |
| 108 | gtgccgaattcggatccagcgatctggattttagtactggattttggttt | RC4260 |
| | taggaattagaaattttattgatagaagtattttacaaatacaaatacat | M. Frank |
| | actaagggtttcttatatgctcaacacatgagcgaaaccctataggaacc | BigPetalA |
| | ctaattcccttatctgggaactactcacacattattatggagaaactcga | +B dCas9- |
| | gcttgtcgatcgactctagctagagaagctcactttccaacaaccttagt | SRDX |
| | aatttctcccttaacataatgaacaaattcttcaagttgatcagctctag | Construct |
| | aagcaagtctatcttcttcttgtccaagataagcttgtctagcttcaaga | (RCGG29 |
| | ataactggttgatattgagctggaagtctttccatagcccaatcagcagc | 93) |
| | aacatcctttggagcaatctttccagtaacagcagaataccaaattctag | |
| | aaagagtaagaacaacatttctttcatctccagcccaatctggtggagaa | |
| | ttccaaagagtaagagtttcattaagagcttcaaaaagatcttgttctgg | |
| | aactggatcaaaaagttcttcagcagctggtccaacaagagcaacagaat | |
| | gttctctagccttagtaagaagaatagcaagatcaatatcaatagtagct | |
| | ggttcaaaaattccagcaagaatatcatttctttgccattctccaaattg | |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcggagtgttcaaaa | |
| | ataaaaagtgattttgagatataatatacatacgacaattaatacaatat | |
| | tatttttgtgtctatccctctcttagaccctattatttatcacatttgaa | |
| | attcagaatttttttttctctcgttgttgtacaaatatctaatcgttaat | |
| | tattattaatgcattaaatatttaaaatataattaatgatattaattttc | |
| | ctagcgtaagactaataataaatatcgacattatgcgagattatgacatg | |
| | atgttatgttaatagtacagaataaagtaaaaaaatatatataacaataa | |
| | taatttaaaatatttaaaatgacatataaagtagttttctaaaacttcag | |
| | tgattagttcaatttattatttcgcaacaattatgatggttaattaaggt | |
| | gaattagaagaagaactgaaaattaagataattaaggagaacaaatgagc | |
| | acttcaaaatttgctattaagccgactctgctagccatgcatgttggatg | |
| | taacttcacccgattgatgggtccttaattataatttttcttatgataaa | |
| | aaataaattgctactaaggcgtactgaactattcgaaatgacaacactac | |
| | aaggcctcgacaacgttaacttttttccaatttagtggggcgttacaagc | |
| | taaagcttttttatttttgaactttaaattgctctttcactttcatttaa | |
| | taacgcctaaacctagctacgagcaatataacctgggtgtatatagctat | |
| | atatatcatatgcagagcaatctgtaaccccaaggataaaacgttgtgtt | |
| | gcttattatagaaatcacagttttaaatggaacgagtgacagagtttgtt | |
| | tgtttctaaataggataggaggcatttaatcggaaattggacaggagaaa | |
| | gttgttttctttctgcaaattatgcctccagaacttgttttacggtaaga | |
| | aaatttacaccaaatttacaagtattggttctgaacgctacttgcggaaa | |
| | ttttatgccgccagtgacttcttggcactttaaataagttaattagttaa | |
| | tcatttgaaaatatattaacaagaagaatttttttctagtgtaatcaaga | |
| | ggtattttaagcttctatcttacactcaattaatcttctattctcctatt | |
| | gattggtctattttaaagtaaagtaatatattcaattacgacttcatgct | |
| | aaattcaaagattataacagtaaaaacaatttttattcacctctattcaa | |
| | actgccagtttagcgcaagtgtttatttttctgataaacttgcggccgca | |
| | caaaacccagcattgcttcaatgtgcataagcatgggtacggtggtggtg | |
| | tttctccaacctccatttcagattgtcttgatttgtaattcaaacacaca | |
| | cttagtcttaaaatcatattttctcatatctcaattgtgatttttccaaa | |
| | tgcgttgtgtgtaggcgaagctaagtttaggttattgctcaagtgccaca | |
| | agtttgagagggttttactgtcttttttatttaatctagaaatatgattt | |
| | ttggacacgtctacttgttacattccgtttacttatggtagtatttgact | |
| | aataaagacattaagattatagtagcatttgactaataaagacacgcgta | |
| | cgtaggtcactattttattggctaagtaaaaaaaaaaaaaaacacgaaaa | |
| | aactttcaaacaaaggggaagcggagaaatcagcctaacttgatattagc | |
| | tgtacaaaatacataaatagctgtcggaaataattatctcttgcagtata | |
| | tatatagaaagtagcagactctaaaatataacatgaagatgtgattaagc | |
| | atttattccattatatggcccataaacatgcatgatgcaaatagaaggat | |
| | gtagggtaaagttgccctaagaaacgaattgaactgcggtcgttgcggta | |
| | tgagaaagagagagagaaaagtgagtgaaggaaggtgggagaatggtttc | |
| | cgtgacggagtaggacggaaagtaagggtccccacagtgcacatgggagc | |
| | ggatttgacgatgagtcgagaaagagcacaagacgtttatgacaaagcct | |
| | gcaatagctttcgttaatttggcaaaagacccctaccatttgcggcaatg | |
| | cacctccccgttctattatttctctttccctcattcattatatcccctat | |
| | tctctgccccagatcgatcgaacaagtgaattagctagaagcattgaagg | |
| | acgtagctgctccatggattacaaagatgacgatgataaagactacaagg | |
| | acgacgacgataaaatggcccctaaaaagaagcgcaaggtgggtattcat | |
| | ggggtgcctgcagctggaatggataagaagtactccatcggactggctat | |
| | cggaactaactccgtgggatgggctgtgatcactgatgagtacaaggtgc | |
| | catccaagaagttcaaggtgctgggaaacactgatagacactccatcaag | |
| | aagaacctgatcggagctctgctgttcgattccggagagactgctgaggc | |
| | tactagactgaagagaactgctagaagaagatacactagaagaaagaaca | |
| | gaatctgctacctgcaagagatcttctccaacgagatggctaaggtggat | |
| | gattccttcttccacagactggaggagtccttcctggtggaggaggataa | |
| | gaagcacgagagacacccaatcttcggaaacatcgtggatgaggtggctt | |
| | accacgagaagtacccaactatctaccacctgagaaagaagctggtggat | |
| | tccactgataaggctgatctgagactgatctacctggctctggctcacat | |
| | gatcaagttcagaggacacttcctgatcgagggagatctgaacccagata | |
| | actccgatgtggataagctgttcatccaactggtgcaaacttacaaccaa | |
| | ctgttcgaggagaacccaatcaacgcttccggagtggatgctaaggctat | |
| | cctgtccgctagactgtccaagtccaggagactggagaacctgatcgctc | |
| | aactgccaggagagaagaagaacggactgttcggaaacctgatcgctctg | |
| | tccctgggactgactccaaacttcaagtccaacttcgatctggctgagga | |
| | tgctaagctgcaactgtccaaggatacttacgatgatgatctggataacc | |
| | tgctggctcaaatcggagatcaatacgctgatctgttcctggctgctaag | |
| | aacctgtccgatgctatcctgctgtccgatatcctgagagtgaacactga | |
| | gatcactaaggctccactgtccgcttccatgatcaagagatacgatgagc | |
| | accaccaagatctgactctgctgaaggctctggtgagacaacaactgcca | |
| | gagaagtacaaggagatcttcttcgatcaatccaagaacggatacgctgg | |
| | atacatcgatggaggagcttcccaagaggagttctacaagttcatcaagc | |
| | caatcctggagaagatggatggaactgaggagctgctggtgaagctgaac | |
| | agagaggatctgctgagaaagcaaagaactttcgataacggatccatccc | |
| | acaccaaatccacctgggagagctgcacgctatcctgaggagacaagagg | |
| | atttctacccattcctgaaggataacagagagaagatcgagaagatcctg | |
| | actttcagaatcccatactacgtgggaccactggctagaggaaactccag | |
| | attcgcttggatgactagaaagtccgaggagactatcactccatggaact | |
| | tcgaggaggtaagtttctgcttctacctttgatatatatataataattat | |
| | cattaattagtagtaatataatatttcaaatatttttttcaaaataaaag | |
| | aatgtagtatatagcaattgcttttctgtagtttataagtgtgtatattt | |
| | taatttataacttttctaatatatgaccaaaatttgttgatgtgcaggtg | |
| | gtggataagggagcttccgctcaatccttcatcgagagaatgactaactt | |
| | cgataagaacctgccaaacgagaaggtgctgccaaagcactccctgctgt | |
| | acgagtacttcactgtgtacaacgagctgactaaggtgaagtacgtgact | |
| | gagggaatgagaaagccagctttcctgtccggagagcaaaagaaggctat | |
| | cgtggatctgctgttcaagactaacagaaaggtgactgtgaagcaactga | |
| | aggaggattacttcaagaagatcgagtgcttcgattccgtggagatctcc | |
| | ggagtggaggatagattcaacgcttccctgggaacttaccacgatctgct | |
| | gaagatcatcaaggataaggatttcctggataacgaggagaacgaggata | |
| | tcctggaggatatcgtgctgactctgactctgttcgaggatagagagatg | |
| | atcgaggagagacttaagacttacgctcacctgttcgatgataaggtgat | |
| | gaagcaactgaagagaagaagatacactggatggggtagactgtccagaa | |
| | agctgatcaacggaatcagagataagcaatccggaaagactatcctggat | |
| | ttcctgaagtccgatggattcgctaacagaaacttcatgcaactgatcca | |
| | cgatgattccctgactttcaaggaggatatccaaaaggctcaagtgtccg | |
| | gacaaggagattccctgcacgagcacatcgctaacctggctggatcccca | |
| | gctatcaagaagggaatcctgcaaactgtgaaggtggtggatgagctggt | |
| | gaaggtgatgggtagacacaagccagagaacatcgtgatcgagatggcta | |
| | gagagaaccaaactactcaaaagggacaaaagaactccagagagagaatg | |
| | aagagaatcgaggagggaatcaaggagctgggatcccaaatcctgaagga | |
| | gcacccagtggagaacactcaactgcaaaacgagaagctgtacctgtact | |
| | acctgcaaaacggaagagatatgtacgtggatcaagagctggatatcaac | |
| | agactgtccgattacgatgtggatgcaatcgtgccacaatccttcctgaa | |
| | ggatgattccatcgataacaaggtgctgactagatccgataagaacagag | |
| | gaaagtccgataacgtgccatccgaggaggtggtgaagaagatgaagaac | |
| | tactggagacaactgctgaacgctaagctgatcactcaaagaaagttcga | |
| | taacctgactaaggctgagagaggaggactgtccgagctggataaggctg | |
| | gattcatcaagagacaactggtggagactagacaaatcactaagcacgtg | |
| | gctcaaatcctggattccagaatgaacactaagtacgatgagaacgataa | |
| | gctgatcagagaggtgaaggtgatcactctgaagtccaagctggtgtccg | |
| | atttcagaaaggatttccaattctacaaggtgagagagatcaacaactac | |
| | caccacgctcacgatgcttacctgaacgctgtggtgggaactgctctgat | |
| | caagaagtacccaaagctggagtccgagttcgtgtacggagattacaagg | |
| | tgtacgatgtgagaaagatgatcgctaagtccgagcaagagatcggaaag | |
| | gctactgctaagtacttcttctactccaacatcatgaacttcttcaagac | |
| | tgagatcactctggctaacggagagatcagaaaaagaccactgatcgaga | |
| | ctaacggagagactggagagatcgtgtgggataagggaagagatttcgct | |
| | actgtgagaaaggtgctgtccatgccacaagtgaacatcgtgaaaaagac | |
| | tgaggtgcaaactggaggattctccaaggagtccatcctgccaaagagaa | |
| | actccgataagctgatcgctagaaagaaggattgggatccaaagaagtac | |
| | ggaggattcgattccccaactgtggcttactccgtgctggtggtggctaa | |
| | ggtggagaagggaaagtccaagaagctgaagtccgtgaaggagctgctgg | |
| | gaatcactatcatggagagatcctccttcgagaagaacccaatcgatttc | |
| | ctggaggctaagggatacaaggaggtgaagaaggatctgatcatcaagct | |
| | gccaaagtactccctgttcgagctggagaacggaagaaagagaatgctgg | |
| | cttccgctggagagctgcaaaagggaaacgagctggctctgccatccaag | |
| | tacgtgaacttcctgtacctggcttcccactacgagaagctgaagggatc | |
| | cccagaggataacgagcaaaagcaactgttcgtggagcaacacaagcact | |
| | acctggatgagatcatcgagcaaatctccgagttctccaagagagtgatc | |
| | ctggctgatgctaacctggataaggtgctgtccgcttacaacaagcacag | |
| | agataagccaatcagagagcaagctgagaacatcatccacctgttcactc | |
| | tgactaacctgggagctccagctgctttcaagtacttcgatactactatc | |
| | gatagaaagagatacacttccactaaggaggtgctggatgctactctgat | |
| | ccaccaatccatcactggactgtacgagactagaatcgatctgtcccaac | |
| | tgggaggagatagttcgaagcgcccagccgctaccaaaaaagctggacag | |
| | gctaaaaaaaagaagggtgacggcagcgggtccggctccgggtccggttc | |
| | cgtcgatctcgatttggatctggaacttcgcctggggttcgcactcgatc | |
| | tcgaccttgaactgcgtcttggctttgctctcgatcttgatctggagttg | |
| | aggcttggattcgcttaggcttgtcaagcagatcgttcaaacatttggca | |
| | ataaagtttcttaagattgaatcctgttgccggtcttgcgatgattatca | |
| | tataatttctgttgaattacgttaagcatgtaataattaacatgtaatgc | |
| | atgacgttatttatgagatgggtttttatgattagagtcccgcaattata | |
| | catttaatacgcgatagaaaacaaaatatagcgcgcaaactaggataaat | |
| | tatcgcgcgcggtgtcatctatgttactagatcgacgctttacgaattcc | |
| | catggggaggacgtcgttgtggttggtgctttccttacattctgagcctc | |
| | tttccttctaatccactcatctgcatcttcttgtgtccttactaatacct | |
| | cattggttccaaattccctccctttaagcaccagctcgtttctgttcttc | |
| | cacagcctcccaagtatccaagggactaaagcctccacattcttcagatc | |
| | aggatattcttgtttaagatgttgaactctatggaggtttgtatgaactg | |
| | atgatctaggaccggataagttcccttcttcatagcgaacttattcaaag | |
| | aatgttttgtgtatcattcttgttacattgttattaatgaaaaaatatta | |
| | ttggtcattggactgaacacgagtgttaaatatggaccaggccccaaata | |
| | agatccattgatatatgaattaaataacaagaataaatcgagtcaccaaa | |
| | ccacttgccttttttaacgagacttgttcaccaacttgatacaaaagtca | |
| | ttatcctatgcaaatcaataatcatacaaaaatatccaataacactaaaa | |
| | aaattaaaagaaatggataatttcacaatatgttatacgataaagaagtt | |
| | acttttccaagaaattcactgattttataagcccacttgcattagataaa | |
| | tggcaaaaaaaaacaaaaaggaaaagaaataaagcacgaagaattctaga | |
| | aaatacgaaatacgcttcaatgcagtgggacccacggttcaattattgcc | |
| | aattttcagctccaccgtatatttaaaaaataaaacgataatgctaaaaa | |
| | aatataaatcgtaacgatcgttaaatctcaacggctggatcttatgacga | |
| | ccgttaaggaaattgtggttgtcggacgaagtccagtaataaacggcgtc | |
| | aaagtggttgcagccggcacacacgagtcgtgtttatcaactcaaagcac | |
| | aaatacttttcctcaacctaaaaataaggcaattagccaaaaacaacttt | |
| | gcgtgtaaacaacgctcaatacacgtgtcattttattattagctattgct | |
| | tcaccgccttagctttctcgtgacctagtcgtcctcgtcttttcttcttc | |
| | ttcttctataaaacaatacccaaagagctcttcttcttcacaattcagat | |
| | ttcaatttctcaaaatcttaaaaactttctctcaattctctctaccgtga | |
| | tcaaggtaaatttctgtgttccttattctctcaaaatcttcgattttgtt | |
| | ttcgttcgatcccaatttcgtatatgttctttggtttagattctgttaat | |
| | cttagatcgtagacgattttctgggtttgatcgttagatatcatcttaat | |
| | tctcgattagggtttcatagatatcatccgatttgttcaaataatttgag | |
| | ttttgtcgaataattactcttcgatttgtgatttctatctagatctggtg | |
| | ttagtttctagtttgtgcgatcgaatttgtcgattaatctgagtttttct | |
| | gattaacagatgcagatcttaatgacaaagcaccagtggtctagtggtag | |
| | aatagtaccctgccacggtacagacccgggttcgattcccggctggtgca | |
| | tggagaatggtgcgcgcagtgttttagagctagaaatagcaagttaaaat | |
| | aaggctagtccgttatcaacttgaaaaagtggcaccgagtcggtgcaaca | |
| | aagcaccagtggtctagtggtagaatagtaccctgccacggtacagaccc | |
| | gggttcgattcccggctggtgcaaagaggagtgacttggttttgttttag | |
| | agctagaaatagcaagttaaaataaggctagtccgttatcaacttgaaaa | |
| | agtggcaccgagtcggtgcaacaaagcaccagtggtctagtggtagaata | |
| | gtaccctgccacggtacagacccgggttcgattcccggctggtgcaactc | |
| | tctctacaagtggtaagttttagagctagaaatagcaagttaaaataagg | |
| | ctagtccgttatcaacttgaaaaagtggcaccgagtcggtgcaacaaagc | |
| | accagtggtctagtggtagaatagtaccctgccacggtacagacccgggt | |
| | tcgattcccggctggtgcacattagatgagtgatataaagttttagagct | |
| | agaaatagcaagttaaaataaggctagtccgttatcaacttgaaaaagtg | |
| | gcaccgagtcggtgcaacaaagcaccagtggtctagtggtagaatagtac | |
| | cctgccacggtacagacccgggttcgattcccggctggtgcaagtttccg | |
| | tgaatcaaaaatgttttagagctagaaatagcaagttaaaataaggctag | |
| | tccgttatcaacttgaaaaagtggcaccgagtcggtgcaacaaagcacca | |
| | gtggtctagtggtagaatagtaccctgccacggtacagacccgggttcga | |
| | ttcccggctggtgcacacatgttctattgtaagatgttttagagctagaa | |
| | atagcaagttaaaataaggctagtccgttatcaacttgaaaaagtggcac | |
| | cgagtcggtgccaacaaagcaccagtggtctagtggtagaatagtaccct | |
| | gccacggtacagacccgggttcgattcccggctggtgcagcttctctagc | |
| | tagagtcgatcgacaagctcgagtttctccataataatgtgtgagtagtt | |
| | cccagataagggaattagggttcctatagggtttcgctcatgtgttgagc | |
| | atataagaaacccttagtatgtatttgtatttgtaaaatacttctatcaa | |
| | taaaatttctaattcctaaaaccaaaatccagtactaaaatccagatcgc | |
| | tcagagaggatgcacatgtgaccgagggacacgaagtgatccgtttaaac | |
| | tatcagtgtttgacaggatatattggcgggtaaacctaagagaaaagagc | |
| | gtttattagaataatcggatatttaaaagggcgtgaaaaggtttatccgt | |
| | tcgtccatttgtatgtgccagccgcctttgcgacgctcaccgggctggtt | |
| | gccctcgccgctgggctggcggccgtctatggccctgcaaacgcgccaga | |
| | aacgccgtcgaagccgtgtgcgagacaccgcggccgccggcgttgtggat | |
| | acctcgcggaaaacttggccctcactgacagatgaggggcggacgttgac | |
| | acttgaggggccgactcacccggcgcggcgttgacagatgaggggcaggc | |
| | tcgatttcggccggcgacgtggagctggccagcctcgcaaatcggcgaaa | |
| | acgcctgattttacgcgagtttcccacagatgatgtggacaagcctgggg | |
| | ataagtgccctgcggtattgacacttgaggggcgcgactactgacagatg | |
| | aggggcgcgatccttgacacttgaggggcagagtgctgacagatgagggg | |
| | cgcacctattgacatttgaggggctgtccacaggcagaaaatccagcatt | |
| | tgcaagggtttccgcccgtttttcggccaccgctaacctgtcttttaacc | |
| | tgcttttaaaccaatatttataaaccttgtttttaaccagggctgcgccc | |
| | tgtgcgcgtgaccgcgcacgccgaaggggggtgcccccccttctegaacc | |
| | ctcccggcccgctaacgcgggcctcccatccccccaggggctgcgcccct | |
| | cggccgcgaacggcctcaccccaaaaatggcagcgctggccaattcccga | |
| | gtgcgcggaacccctatttgtttatttttctaaatacattcaaatatgta | |
| | tccgctcatgagacaataaccctgataaatgcttcaataatattgaaaaa | |
| | ggaagagtatggctaaaatgagaatatcaccggaattgaaaaaactgatc | |
| | gaaaaataccgctgcgtaaaagatacggaaggaatgtctcctgctaaggt | |
| | atataagctggtgggagaaaatgaaaacctatatttaaaaatgacggaca | |
| | gccggtataaagggaccacctatgatgtggaacgggaaaaggacatgatg | |
| | ctatggctggaaggaaagctgcctgttccaaaggtcctgcactttgaacg | |
| | gcatgatggctggagcaatctgctcatgagtgaggccgatggcgtccttt | |
| | gctcggaagagtatgaagatgaacaaagccctgaaaagattatcgagctg | |
| | tatgcggagtgcatcaggctctttcactccatcgacatatcggattgtcc | |
| | ctatacgaatagcttagacagccgcttagccgaattggattacttactga | |
| | ataacgatctggccgatgtggattgcgaaaactgggaagaggacactcca | |
| | tttaaagatccgcgcgagctgtatgattttttaaagacggaaaagcccga | |
| | agaggaacttgtcttttcccacggcgacctgggagacagcaacatctttg | |
| | tgaaagatggcaaagtaagtggctttattgatcttgggagaagcggcagg | |
| | gcggacaagtggtatgacattgccttctgcgtccggtcgctcagggagga | |
| | tatcggggaagaacagtatgtcgagctattttttgacttactggggatca | |
| | agcctgattgggagaaaataaaatattatattttactggatgaattgttt | |
| | tagctgtcagaccaagtttactcatatatactttagattgatttaaaact | |
| | tcatttttaatttaaaaggatctaggtgaagatcctttttgataatctca | |
| | tgaccaaaatcccttaacgtgagttttcgttccactgagcgtcagacccc | |
| | gtagaaaagatcaaaggatcttcttgagatcctttttttctgcgcgtaat | |
| | ctgctgcttgcaaacaaaaaaaccaccgctaccagcggtggtttgtttgc | |
| | cggatcaagagctaccaactctttttccgaaggtaactggcttcagcaga | |
| | gcgcagataccaaatactgtccttctagtgtagccgtagttaggccacca | |
| | cttcaagaactctgtagcaccgcctacatacctcgctctgctaatcctgt | |
| | taccagtggctgctgccagtggcgataagtcgtgtcttaccgggttggac | |
| | tcaagacgatagttaccggataaggcgcagcggtcgggctgaacgggggg | |
| | ttcgtgcacacagcccagcttggagcgaacgacctacaccgaactgagat | |
| | acctacagcgtgagctatgagaaagcgccacgcttcccgaagggagaaag | |
| | gcggacaggtatccggtaagcggcagggtcggaacaggagagcgcacgag | |
| | ggagcttccagggggaaacgcctggtatctttatagtcctgtcgggtttc | |
| | gccacctctgacttgagcgtcgatttttgtgatgctcgtcaggggggcgg | |
| | agcctatggaaaaacgccagcaacgcggcctttttacggttcctggcaga | |
| | tcctagatgtggcgcaacgatgccggcgacaagcaggagcgcaccgactt | |
| | cttccgcatcaagtgttttggctctcaggccgaggcccacggcaagtatt | |
| | tgggcaaggggtcgctggtattcgtgcagggcaagattcggaataccaag | |
| | tacgagaaggacggccagacggtctacgggaccgacttcattgccgataa | |
| | ggtggattatctggacaccaaggcaccagggggtcaaatcaggaataagg | |
| | gcacattgccccggcgtgagtcggggcaatcccgcaaggagggtgaatga | |
| | atcggacgtttgaccggaaggcatacaggcaagaactgatcgacgcgggg | |
| | ttttccgccgaggatgccgaaaccatcgcaagccgcaccgtcatgcgtgc | |
| | gccccgcgaaaccttccagtccgtcggctcgatggtccagcaagctacgg | |
| | ccaagatcgagcgcgacagcgtgcaactggctccccctgccctgcccgcg | |
| | ccateggccgccgtggagcgttcgcgtcgtcttgaacaggaggcggcagg | |
| | tttggcgaagtcgatgaccatcgacacgcgaggaactatgacgaccaaga | |
| | agcgaaaaaccgccggcgaggacctggcaaaacaggtcagcgaggccaag | |
| | caggccgcgttgctgaaacacacgaagcagcagatcaaggaaatgcagct | |
| | ttccttgttcgatattgcgccgtggccggacacgatgcgagcgatgccaa | |
| | acgacacggcccgctctgccctgttcaccacgcgcaacaagaaaatcccg | |
| | cgcgaggcgctgcaaaacaaggtcattttccacgtcaacaaggacgtgaa | |
| | gatcacctacaccggcgtcgagctgcgggccgacgatgacgaactggtgt | |
| | ggcagcaggtgttggagtacgcgaagcgcacccctatcggcgagccgatc | |
| | accttcacgttctacgagctttgccaggacctgggctggtcgatcaatgg | |
| | ccggtattacacgaaggccgaggaatgcctgtcgcgcctacaggcgacgg | |
| | cgatgggcttcacgtccgaccgcgttgggcacctggaatcggtgtcgctg | |
| | ctgcaccgcttccgcgtcctggaccgtggcaagaaaacgtcccgttgcca | |
| | ggtcctgatcgacgaggaaatcgtcgtgctgtttgctggcgaccactaca | |
| | cgaaattcatatgggagaagtaccgcaagctgtcgccgacggcccgacgg | |
| | atgttcgactatttcagctcgcaccgggagccgtacccgctcaagctgga | |
| | aaccttccgcctcatgtgoggatcggattccacccgcgtgaagaagtggc | |
| | gcgagcaggtcggcgaagcctgcgaagagttgcgaggcagcggcctggtg | |
| | gaacacgcctgggtcaatgatgacctggtgcattgcaaacgctagggcct | |
| | tgtggggtcagttccggctgggggttcagcagcccctgctcggatctgtt | |
| | ggaccggacagtagtcatggttgatgggctgcctgtatcgagtggtgatt | |
| | ttgtgccgagctgccggtcggggagctgttggctggctggtggcaggata | |
| | tattgtggtgtaaacaaattgacgcttagacaacttaataacacattgcg | |
| | gacgtttttaatgtactggggttgaacactct | |
| 109 | gtgccgaattcggatccagcgatctggattttagtactggattttggttt | RC4272 |
| | taggaattagaaattttattgatagaagtattttacaaatacaaatacat | M. Frank |
| | actaagggtttcttatatgctcaacacatgagcgaaaccctataggaacc | TA29ab |
| | ctaattcccttatctgggaactactcacacattattatggagaaactcga | CONTROL |
| | gcttgtcgatcgactctagctagagaagctcactttccaacaaccttagt | Construct |
| | aatttctcccttaacataatgaacaaattcttcaagttgatcagctctag | (RCGG30 |
| | aagcaagtctatcttcttcttgtccaagataagcttgtctagcttcaaga | 05) |
| | ataactggttgatattgagctggaagtctttccatagcccaatcagcagc | Likely |
| | aacatcctttggagcaatctttccagtaacagcagaataccaaattctag | same as |
| | aaagagtaagaacaacatttctttcatctccagcccaatctggtggagaa | SEQ ID |
| | ttccaaagagtaagagtttcattaagagcttcaaaaagatcttgttctgg | NO: 58 |
| | aactggatcaaaaagttcttcagcagctggtccaacaagagcaacagaat | |
| | gttctctagccttagtaagaagaatagcaagatcaatatcaatagtagct | |
| | ggttcaaaaattccagcaagaatatcatttctttgccattctccaaattg | |
| | aagttctctcttagctggatatctccatggaataatatcatcatgaacaa | |
| | caatagtaacttcaacagctctaagaatttcagattctcctggagaagca | |
| | gaagtttcaagaagatcattaataagagctcttctagtagtttcatcaag | |
| | tctaacagtaacagtaacaagaagatcaatatcagaatgtggcttaagtc | |
| | ctccatcaacagcagatccataaagatgaacagcaagaagagtaggttca | |
| | agatgtctttcaataactccaacaacttcagaaagttgagtagaaacttc | |
| | agcaataacagcttctctcatgcaagcagtagcaacagaagcagaaattc | |
| | tgaaagaacagaacttttgcatgaaaatagaatccttcttaagaacaagc | |
| | atagaattagcagagttcttaagcttcttagatccgaaaacaagaaaaga | |
| | agatgacttaggaacttgtggcttatggaaattagagttaggattaagag | |
| | tttgaattccttgagccatattgttaatttgagccattgtatcgataatt | |
| | gtaaatgtaattgtaatgttgtttgttgtttgttgttgttggtaattgtt | |
| | gtaaaaatgagctcttatactcgagcgtgtcctctccaaatgaaatgaac | |
| | ttccttatatagaggaagggtcttgcgaaggatagtgggattgtgcgtca | |
| | tcccttacgtcagtggagatgtcacatcaatccacttgctttgtagacgt | |
| | ggttggaacctcttctttttccacgatgctcctcgtggggggggtccatc | |
| | tttgggaccactgtcggcagagagatcttgaatgatagcctttcctttat | |
| | cgcaatgatggcatttgtaggagccaccttccttttctactgtcctttcg | |
| | atgaagtgacagatagctgggcaatggaatccgaggaggtttcccgaaat | |
| | tatcctttgttgaaaagtctcaatagccctttgatcttctgagactgtat | |
| | ctttgacatttttggagtagaccagagtgtcgtgctccaccatgttatca | |
| | catcaatccacttgctttgtagacgtggttggaacctcttctttttccac | |
| | gatgctcctcgtggggggggtccatctttgggaccactgtcggcagagag | |
| | atcttgaatgatagcctttcctttatcgcaatgatggcatttgtaggagc | |
| | caccttccttttctactgtcctttcgatgaagtgacagatagctgggcaa | |
| | tggaatccgaggaggtttcccgaaattatcctttgttgaaaagtctcaat | |
| | agccctttgatcttctgagactgtatctttgacatttttggagtagacca | |
| | gagtgtcgtgctccaccatgttgacctccgcaagaattcaagcttagcgt | |
| | tcctaacacctggagaaccttttatgtacttcacaaccctcaatgctgct | |
| | tccaggtgtgactgtttgggttgctgcatgaattgactcagcacctgcac | |
| | tgcaaagctgatatctggccttgtgattgtcaagtagagaagctttccca | |
| | ccagtctctgataggatcccacatcctctaactctgcatcatcagtcttt | |
| | cctaagtgcttgtcatattctacagtagtcagcctttggttctgttccat | |
| | tggggttgacactggtctgcagcctcccagaccaacacctgatatcagtt | |
| | ccagtgcatacttcctctggttcagtaggattcccttttctgatctcagc | |
| | acctcaatgcctaagaagtattttagttctcccaaatctttcatcttgaa | |
| | atgctgatgcagggttgcctttgcttctgaaatcaaaacattgctgctgc | |
| | ctgttattaacagatcatccacataaatcaggattatgacaaggtcagtc | |
| | ccctctcttttggtaaacaaggagtgatcataagcactttgcataaaacc | |
| | agcctgcataaggacagtggtaagcttgatgttccactgccttgatgctt | |
| | gctttaaaccatagagggattcaacagcctgcacactttggtttcccctt | |
| | ggctgtgaaaaccctgaggcagagacatataaacttcttccatgaggtca | |
| | ccttgtagaaaagcattgttgacatccatctggaaaaggaaccagccctt | |
| | ggaagcagcaacagatatgacagcttttacagtgaccattttggccactg | |
| | gagaaaaagtttcatggtagtcaaggccttcttgctgagtgtatcccttg | |
| | gccactagccttgccttaaacctgtcaacttcaccattagctttgtattt | |
| | aattttgtacacccatttggaccctataggctgtttaccagggggtaaag | |
| | ggacaatctcccaggtgttattatcctcaagagcctgtatctcaagggac | |
| | atggcctccatccatttctcatcttgagctgcttctttgaaggatttagg | |
| | ttcagtataagtggagaaagcactcaaataagcttgatagtgaacaggca | |
| | agtgatcataggaaacatagttggctatagggtatggaacatccctagag | |
| | cctttgttcagtgtcacaaagtccttgagccagatgggaggacctgcatt | |
| | gcgtttaggtctagtgtgcaggtttgctggaactagagagggatcagcta | |
| | cagcagtatggtgctcaaattcagcatttgctaggtcaggctcagctgac | |
| | tcaactgactctgcaggagcatgcaggtggcctgaaggtgcagcatcagc | |
| | tgaagtgatatgattttgggtagaatgagtccccaaagtgatatcctcat | |
| | tggcctctacaatgtcaggaataagaactggagtatcatcatcatcagta | |
| | tgaaagctggaaggaaaaatatcattatacactagctgcaaagctgtatc | |
| | ttcagagctaagtgaacctgctcgagtgaacatatcaggttcatgggaga | |
| | tggactctttgaaagggaactgaaactctctgaaaactacatccctgctc | |
| | actatgatcaccttattatccaagtcatacaacctgtaacccttttgagt | |
| | ctcagaataaccaatgaagatggttcgcttggctctaggtgcaagtttgt | |
| | cacctttgggcagtgttgctgcaaaagcaagacacccaaacactctcaag | |
| | tgatcaagcttggcatgtttctgatagagtagttcatatggacatttgcc | |
| | ttgtaagattggagtagggagtctattgatcaggtatacaacagtcttga | |
| | cacagtctccccaaaacctggtaggtacaccactctgaaacttaagtgcc | |
| | cttgccatctcaaggatgtgtctgtgctttctctccacaacaccattctg | |
| | ttgtggtgtgtagggacagctactttgatgaacaatcccaagagaggcca | |
| | gcaactcattacaacttctccactagaattcgagctcagcgtcgatctag | |
| | taacatagatgacaccgcgcgcgataatttatcctagtttgcgcgctata | |
| | ttttgttttctatcgcgtattaaatgtataattgcgggactctaatcata | |
| | aaaacccatctcataaataacgtcatgcattacatgttaattattacatg | |
| | cttaacgtaattcaacagaaattatatgataatcatcgcaagaccggcaa | |
| | caggattcaatcttaagaaactttattgccaaatgtttgaacgatctgct | |
| | tgacaagcttagaccttccgcttcttctttggcgaacttggaagggcgga | |
| | gccggaagcgatagcgtgctcggtaaggtgccacttctggttcttagcat | |
| | eggacctatcctccctggtaagcttgtgctggatgaagtgccaatctggc | |
| | atcttccttggcacggacttagccttgtacacggtatcgaactggcacct | |
| | aagcctgccgccatccttaagaagaaggtacatggacacatcgcccttaa | |
| | ggatgccctgctttggcactgggatgatcttctcgcaggatggctcccag | |
| | ttatcggtcatcttcttcatcactgggccatcagctgggaagttcacgcc | |
| | gtagaacttggactcgtggtacatgcagttctcctccacggacacggtga | |
| | tatcagcgttgcagatgcacacagcgccatcctcgaaaaggaaggaccta | |
| | tcccaggtgtagccagctgggcaggagttcttgaagtaatcagcgatatc | |
| | ctgtgggtactcggtgaacaccctgttgccgtacatgaaagcagcggaaa | |
| | ggatatcctcagcgaatggaagtgggccgccctccaccacgcaaaggttg | |
| | atagcctgtttgcccttgaatgggtagccgatgccctcgccggtgatcac | |
| | gaacttgtggccatccacgcagccctccatcctgtacttcatggtcatct | |
| | ccttggtaaggccgtgcttggactgagccattgacttcaatttaaataac | |
| | ttttggttattaattaatttgtttactaatcccactgtatatgattgtgt | |
| | atgcatggtttgcattgagtagtgtccctatttatggtgaattttggaag | |
| | caaattggcttgttcatttcgtgagtcacctgccttgagaggttttctga | |
| | tcataactttggtgttggatccatggcaactgcatgtgacatgaggatga | |
| | tcatgtgttcaatcctttttcttgtctaccttggcttatctccactttct | |
| | atcaagtccgatatatagttctagtatagtttaattaggttaatgtgtgc | |
| | acatatttgctaacacttagggtatatgtggcagaataaaaacataggca | |
| | tggatagacatggataaaatttatgcaatggaaataaattatatgttttt | |
| | ttttttacaaataactagagagaaaggaagagaatagagaaaaataataa | |
| | tataacatatgatatatgtaataaataaattaagtgttatgaaaaatatt | |
| | aaaaaaattgaatggaaaaaaatttcaagtgttaaacaatatatacctac | |
| | cactggattaaccattacttattaaaatatggaattgaatttatttggaa | |
| | aaaaacctggacattttttaaagctgtttttatttaaaagctaaaaaaat | |
| | tatgttgtttgataaaaatagtttcgaaaactgtttttaaaactactatc | |
| | tctttttttagttttttcaaaaccaaaaaactataacatctctatgatcg | |
| | atgtttgggttttttacttttaattttctattattcatgttcttccttgt | |
| | attttcacatcttacttttccacttttactctctaaacatcagtttatga | |
| | aaaatgtttcataaaatagtttttaaaaaatagactaagaaattaaaaca | |
| | caattaaacacactcttaaaaatacttaaaaattatttaacatgtaaaat | |
| | attattttgagaatgatttataaatactcattatgcttaatattgattaa | |
| | aacactgagtaaaataaaaacttgaaaaaatagtttttttttgttgttga | |
| | ataattagttagagaggaagaaaattctaatttgaattgatataaatgac | |
| | cataaaatatgtttcaaaaataaaaaaaagacacatgactgcataaaaat | |
| | gaaatacctaaaataaaaattttatttcaagaataaaaacttgaatttgt | |
| | actgtatgaatatattataacgttaaaatcttaaccatttatttattaat | |
| | aattataacttttattcagtaaaatgcagttctaacttaaagtcaagtta | |
| | attttatataaattactttcctaatttggagaaatgattgatgcatatat | |
| | cctttgaaatcacatatccccacttttgggttaggctgcataattgaagt | |
| | ggggcttgcaaaatatgagttacaggcaaccataattactgtgttgaaat | |
| | ttcagttggaaaagttcaaaactatagtttaaataaaatcattagataga | |
| | tttttacgtaaatcataatattacaaactccttacgaattcccatggagc | |
| | gtccctcggtcacatgtgcatcctctagtggagaagttgtaatgagttgc | |
| | tggcctctcttgggattgttcatcaaagtagctgtccctacacaccacaa | |
| | cagaatggtgttgtggagagaaagcacagacacatccttgagatggcaag | |
| | ggcacttaagtttcagagtggtgtacctaccaggttttggggagactgtg | |
| | tcaagactgttgtatacctgatcaatagactccctactccaatcttacaa | |
| | ggcaaatgtccatatgaactactctatcagaaacatgccaagcttgatca | |
| | cttgagagtgtttgggtgtcttgcttttgcagcaacactgcccaaaggtg | |
| | acaaacttgcacctagagccaagcgaaccatcttcattggttattctgag | |
| | actcaaaagggttacaggttgtatgacttggataataaggtgatcatagt | |
| | gagcagggatgtagttttcagagagtttcagttccctttcaaagagtcca | |
| | tctcccatgaacctgatatgttcactcgagcaggttcacttagctctgaa | |
| | gatacagctttgcagctagtgtataatgatatttttccttccagctttca | |
| | tactgatgatgatgatactccagttcttattcctgacattgtagaggcca | |
| | atgaggatatcactttggggactcattctacccaaaatcatatcacttca | |
| | gctgatgctgcaccttcaggccacctgcatgctcctgcagagtcagttga | |
| | gtcagctgagcctgacctagcaaatgctgaatttgagcaccatactgctg | |
| | tagctgatccctctctagttccagcaaacctgcacactagacctaaacgc | |
| | aatgcaggtcctcccatctggctcaaggactttgtgacactgaacaaagg | |
| | ctctagggatgttccataccctatagccaactatgtttcctatgatcact | |
| | tgcctgttcactatcaagcttatttgagtgctttctccacttatactgaa | |
| | cctaaatccttcaaagaagcagctcaagatgagaaatggatggaggccat | |
| | gtcccttgagatacaggctcttgaggataataacacctgggagattgtcc | |
| | ctttaccccctggtaaacagcctatagggtccaaatgggtgtacaaaatt | |
| | aaatacaaagctaatggtgaagttgacaggtttaaggcaaggctagtggc | |
| | caagggatacactcagcaagaaggccttgactaccatgaaactttttctc | |
| | cagtggccaaaatggtcactgtaaaagctgtcatatctgttgctgcttcc | |
| | aagggctggttccttttccagatggatgtcaacaatgcttttctacaagg | |
| | tgacctcatggaagaagtttatatgtctctgcctcagggttttcacagcc | |
| | aaggggaaaccaaagtgtgcaggctgttgaatccctctatggtttaaagc | |
| | aagcatcaaggcagtggaacatcaagcttaccactgtccttatgcaggct | |
| | ggttttatgcaaagtgcttatgatcactccttgtttaccaaaagagaggg | |
| | gactgaccttgtcataatcctgatttatgtggatgatctgttaataacag | |
| | gcagcagcaatgttttgatttcagaagcaaaggcaaccctgcatcagcat | |
| | ttcaagatgaaagatttgggagaactaaaatacttcttaggcattgaggt | |
| | gctgagatcagaaaagggaatcctactgaaccagaggaagtatgcactgg | |
| | aactgatatcaggtgttggtctgggaggctgcagaccagtgtcaacccca | |
| | atggaacagaaccaaaggctgactactgtagaatatgacaagcacttagg | |
| | aaagactgatgatgcagagttagaggatgtgggatcctatcagagactgg | |
| | tgggaaagcttctctacttgacaatcacaaggccagatatcagctttgca | |
| | gtgcaggtgctgagtcaattcatgcagcaacccaaacagtcacacctgga | |
| | agcagcattgagggttgtgaagtacataaaaggttctccaggtgttagga | |
| | acgctaagcttgaattcttgcggaggtttccatagcctaaggccacgagt | |
| | gcacgtgttcataggctagttgtgttaaccttggggagcagtcgacaatt | |
| | tggattgcatcgaggtcttgccaaattttactttcaagacagtgatttcg | |
| | acggcacaataaacttccaattagttatcttctcttttgttctgtattgt | |
| | gtttttttctatttttctaacaatggaaatagtatataccggccaacagt | |
| | gttgcactgttgtctggaccttgtcaaaggttcgacagtgcatagtttgg | |
| | actcgatgtggcatatgggggttctgataagtgatgagattatgaaaata | |
| | atttaattttagctattaaaaatacaatacaactttcaaaagcgggaaac | |
| | ttgtttgattatgatctaggattctaggtctttttttttaagttcgagac | |
| | agaaaaccaaaaacaaaacagctaataagaagaagaaaaacaatacagca | |
| | atattcccacaaaaataaatttgttcttttgcgtactagccataggattt | |
| | aattatatatatgggccagtccagaagcattgtcactgatagatcataga | |
| | ctagtaaaaaagaatgaaccacttcagaacactaactatatatataaaga | |
| | tagattcatatctatttagatcgtgattattaatacaagaaaatatttgt | |
| | atttcttaattttttgtttttaaatttatgtaaatatattgacatctttt | |
| | ttaacgaaaattcaaattttaattcattatattacaagaaattaacacat | |
| | tttactagaccaactctcatgttacaacactatatatgttgaacattaca | |
| | agaatgccactttatctcacccctttaattaggaaaagcaaaaatctatt | |
| | tgcatataattttgtaccaaaaaattatacaataaaaaagaaaaagagaa | |
| | aagtaaaagataagacgactgatgtaataaaaagagagaaaaaataatac | |
| | taaaaaataatataaataacataactcttaaaaaaaaaaggtaacactgt | |
| | tgagtattttttttattacttttctctaaaaaggtatcatggtttcacta | |
| | atgtccaggttttttttttccaaataaactectttccggtggtaggtatc | |
| | gtttcacacttgaatacttttttgcaatatttttcgtaacactttatttc | |
| | tctccattacataattttacccttgcctatgtctttattctgccacatat | |
| | accctaagagtgagcaaatgtgtgcacgcatataacctgattggactata | |
| | tatcggacttgatacaaatgtgtgctcacatgaagagaaaaaggattgaa | |
| | cccatgatcatcatcatgtcacatgcagatgccatgcattcaacaccaaa | |
| | gttatgatcagaaaacctctcaaggtaggtgactcaccaaatgaacaagc | |
| | caattttttgcttcccaaatttcaccataaatagggacactactccatcc | |
| | aaaccatacacgaccacatacagtgggattagtaagtaaacaaattaatt | |
| | aataaccaaaagttataatttcaattgaatttagtcaatggtgtccaagg | |
| | gcgaagaggtgattaaagagttcatgaggttcaaagtgagaatggagggc | |
| | agtatgaacggccatgagttcgagatcgaaggtgaaggcgaaggtagacc | |
| | atacgagggcacccagacggcgaaactgaaagtgacgaagggcggcccac | |
| | tgcccttcgcctgggacattctgagtccacaatttatgtacggttccaag | |
| | gcatacgttaagcaccccgccgacatccctgattacaaaaagctgtcgtt | |
| | cccggaggggtttaaatgggaaagagtgatgaattttgaggacggagggc | |
| | tcgtcactgtgactcaagactcctcacttcaagacggcaccctaatttac | |
| | aaggtgaagatgaggggaacaaatttcccccctgatggccctgttatgca | |
| | gaaaaaaacaatgggatgggaggcatccaccgagagactttacccgcgcg | |
| | acggagtgctcaaaggcgaaatccatcaggcactgaaactaaaggacggc | |
| | ggccactacctggttgagttcaaaactatatatatggccaagaaacccgt | |
| | ccagttgccgggatattactatgtggacacaaaactggatataacgtccc | |
| | acaacgaagattacaccatagtggaacagtatgagcgcagtgagggtcgt | |
| | caccatctattcctcggacatggcacgggttccacgggctcgggttccag | |
| | cggcacggcaagctcggaggacaacaacatggcggtgatcaaagagttca | |
| | tgcggtttaaagtccgtatggagggcagcatgaatgggcacgaatttgaa | |
| | attgagggagaaggcgagggccgaccatatgaggggacacaaaccgcgaa | |
| | actaaaagtcaccaaaggtgggccgcttccttttgcctgggacattctct | |
| | ctcctcagttcatgtatggctccaaggcctacgttaaacatccggctgat | |
| | atccccgattataagaaattgtctttcccggaggggtttaaatgggagcg | |
| | ggttatgaatttcgaggatggtggactggttactgttacacaagattcga | |
| | gtctgcaggatggcacattgatatacaaagtcaaaatgaggggtacgaat | |
| | tttcccccggacggcccggtgatgcagaaaaaaactatgggctgggaggc | |
| | cagcacggagaggctgtacccgagggacggggttttgaaaggggagatac | |
| | accaggcgcttaaactgaaggacggggggcactatctggtggaatttaag | |
| | acgatatatatggcgaaaaagcccgtgcagctacctggctactactacgt | |
| | cgataccaaactcgatattacctctcataacgaggactacactatcgtgg | |
| | agcaatatgaaagatcagaaggacgtcaccacctttttctatacggaatg | |
| | gacgagctttacaaaagttcgccaaagaagaagcggaaggtctaagcttg | |
| | tgtgtcttgtcttatctggttcgtggtggtgagtttgttacaaaaaaatc | |
| | tattttccctagttgagatgggaattgaactatctgttgttatgtggatt | |
| | ttattttcttttttctctttagaaccttatggttgtgtcaagaagtcttg | |
| | tgtactttagttttatatctctgttttatctcttctattttctttaggat | |
| | gcttgtgatgatgctgtttttttttgtccctaagcaaaaaaatatcatat | |
| | tatatttggtccttggttcatttttttggtttttttttatcttcacatat | |
| | aaatattgtttgaatatcttcaatcttttatttgtatgagacaattattt | |
| | aagtatcgggtgacaatgcagctattatgtattgtcgattgttcgctgga | |
| | tccgaattcggcactcccagagaggatgcacatgtgaccgagggacacga | |
| | agtgatccgtttaaactatcagtgtttgacaggatatattggcgggtaaa | |
| | cctaagagaaaagagcgtttattagaataatcggatatttaaaagggcgt | |
| | gaaaaggtttatccgttcgtccatttgtatgtgccagccgcctttgcgac | |
| | gctcaccgggctggttgccctcgccgctgggctggcggccgtctatggcc | |
| | ctgcaaacgcgccagaaacgccgtcgaagccgtgtgcgagacaccgcggc | |
| | cgccggcgttgtggatacctcgcggaaaacttggccctcactgacagatg | |
| | aggggcggacgttgacacttgaggggccgactcacccggcgcggcgttga | |
| | cagatgaggggcaggctcgatttcggccggcgacgtggagctggccagcc | |
| | tcgcaaatcggcgaaaacgcctgattttacgcgagtttcccacagatgat | |
| | gtggacaagcctggggataagtgccctgcggtattgacacttgaggggcg | |
| | cgactactgacagatgaggggcgcgatccttgacacttgaggggcagagt | |
| | gctgacagatgaggggcgcacctattgacatttgaggggctgtccacagg | |
| | cagaaaatccagcatttgcaagggtttccgcccgtttttcggccaccgct | |
| | aacctgtcttttaacctgottttaaaccaatatttataaaccttgttttt | |
| | aaccagggctgcgccctgtgcgcgtgaccgcgcacgccgaaggggggtgc | |
| | ccccccttctcgaaccctcccggcccgctaacggggcctcccatcccccc | |
| | aggggctgcgcccctcggccgegaacggcctcaccccaaaaatggcagcg | |
| | ctggccaattcccgagtgcgcggaacccctatttgtttatttttctaaat | |
| | acattcaaatatgtatccgctcatgagacaataaccctgataaatgcttc | |
| | aataatattgaaaaaggaagagtatggctaaaatgagaatatcaccggaa | |
| | ttgaaaaaactgatcgaaaaataccgctgcgtaaaagatacggaaggaat | |
| | gtctcctgctaaggtatataagctggtgggagaaaatgaaaacctatatt | |
| | taaaaatgacggacagccggtataaagggaccacctatgatgtggaacgg | |
| | gaaaaggacatgatgctatggctggaaggaaagctgcctgttccaaaggt | |
| | cctgcactttgaacggcatgatggctggagcaatctgctcatgagtgagg | |
| | ccgatggcgtcctttgctcggaagagtatgaagatgaacaaagccctgaa | |
| | aagattatcgagctgtatgcggagtgcatcaggctctttcactccatcga | |
| | catatcggattgtccctatacgaatagcttagacagccgcttagccgaat | |
| | tggattacttactgaataacgatctggccgatgtggattgcgaaaactgg | |
| | gaagaggacactccatttaaagatccgcgcgagctgtatgattttttaaa | |
| | gacggaaaagcccgaagaggaacttgtcttttcccacggcgacctgggag | |
| | acagcaacatctttgtgaaagatggcaaagtaagtggctttattgatott | |
| | gggagaagcggcagggcggacaagtggtatgacattgccttctgcgtccg | |
| | gtcgctcagggaggatatcggggaagaacagtatgtcgagctattttttg | |
| | acttactggggatcaagcctgattgggagaaaataaaatattatatttta | |
| | ctggatgaattgttttagctgtcagaccaagtttactcatatatacttta | |
| | gattgatttaaaacttcatttttaatttaaaaggatctaggtgaagatcc | |
| | tttttgataatctcatgaccaaaatcccttaacgtgagttttcgttccac | |
| | tgagcgtcagaccccgtagaaaagatcaaaggatcttcttgagatccttt | |
| | ttttctgcgcgtaatctgctgcttgcaaacaaaaaaaccaccgctaccag | |
| | cggtggtttgtttgccggatcaagagctaccaactctttttccgaaggta | |
| | actggcttcagcagagcgcagataccaaatactgtccttctagtgtagcc | |
| | gtagttaggccaccacttcaagaactctgtagcaccgcctacatacctcg | |
| | ctctgctaatcctgttaccagtggctgctgccagtggcgataagtcgtgt | |
| | cttaccgggttggactcaagacgatagttaccggataaggcgcagcggtc | |
| | gggctgaacggggggttcgtgcacacagcccagcttggagcgaacgacct | |
| | acaccgaactgagatacctacagcgtgagctatgagaaagcgccacgctt | |
| | cccgaagggagaaaggcggacaggtatccggtaagcggcagggtcggaac | |
| | aggagagcgcacgagggagcttccagggggaaacgcctggtatctttata | |
| | gtcctgtcgggtttcgccacctctgacttgagcgtcgatttttgtgatgc | |
| | tcgtcaggggggggagcctatggaaaaacgccagcaacgcggccttttta | |
| | cggttcctggcagatcctagatgtggcgcaacgatgccggcgacaagcag | |
| | gagcgcaccgacttcttccgcatcaagtgttttggctctcaggccgaggc | |
| | ccacggcaagtatttgggcaaggggtcgctggtattcgtgcagggcaaga | |
| | ttcggaataccaagtacgagaaggacggccagacggtctacgggaccgac | |
| | ttcattgccgataaggtggattatctggacaccaaggcaccaggcgggtc | |
| | aaatcaggaataagggcacattgccccggcgtgagtcggggcaatcccgc | |
| | aaggagggtgaatgaatcggacgtttgaccggaaggcatacaggcaagaa | |
| | ctgatcgacgcggggttttccgccgaggatgccgaaaccatcgcaagccg | |
| | caccgtcatgcgtgcgccccgcgaaaccttccagtccgtcggctcgatgg | |
| | tccagcaagctacggccaagatcgagcgcgacagcgtgcaactggctccc | |
| | cctgccctgcccgcgccatcggccgccgtggagcgttcgcgtcgtcttga | |
| | acaggaggcggcaggtttggcgaagtcgatgaccatcgacacgcgaggaa | |
| | ctatgacgaccaagaagcgaaaaaccgccggcgaggacctggcaaaacag | |
| | gtcagcgaggccaagcaggccgcgttgctgaaacacacgaagcagcagat | |
| | caaggaaatgcagctttccttgttcgatattgcgccgtggccggacacga | |
| | tgcgagcgatgccaaacgacacggcccgctctgccctgttcaccacgcgc | |
| | aacaagaaaatcccgcgcgaggcgctgcaaaacaaggtcattttccacgt | |
| | caacaaggacgtgaagatcacctacaccggcgtcgagctgcgggccgacg | |
| | atgacgaactggtgtggcagcaggtgttggagtacgcgaagcgcacccct | |
| | atcggcgagccgatcaccttcacgttctacgagctttgccaggacctggg | |
| | ctggtcgatcaatggccggtattacacgaaggccgaggaatgcctgtcgc | |
| | gcctacaggcgacggcgatgggcttcacgtccgaccgcgttgggcacctg | |
| | gaatcggtgtcgctgctgcaccgcttccgcgtcctggaccgtggcaagaa | |
| | aacgtcccgttgccaggtcctgatcgacgaggaaatcgtcgtgctgtttg | |
| | ctggcgaccactacacgaaattcatatgggagaagtaccgcaagctgtcg | |
| | ccgacggcccgacggatgttcgactatttcagctcgcaccgggagccgta | |
| | cccgctcaagctggaaaccttccgcctcatgtgcggatcggattccaccc | |
| | gcgtgaagaagtggcgcgagcaggtcggcgaagcctgcgaagagttgcga | |
| | ggcagcggcctggtggaacacgcctgggtcaatgatgacctggtgcattg | |
| | caaacgctagggccttgtggggtcagttccggctgggggttcagcagccc | |
| | ctgctcggatctgttggaccggacagtagtcatggttgatgggctgcctg | |
| | tatcgagtggtgattttgtgccgagctgccggtcggggagctgttggctg | |
| | gctggtggcaggatatattgtggtgtaaacaaattgacgcttagacaact | |
| | taataacacattgcggacgtttttaatgtactggggttgaacactct | |

What is claimed is:

1. A genetically modified legume plant (Fabaceae) cell, legume plant, part thereof, or legume plant seed thereof comprising a reversible sterility phenotype, the genetically modified plant comprising:
a. a sterility expression construct comprising a promoter operably linked to a sterility polynucleotide encoding a sterility factor;
b. a fertility restorer expression construct comprising a promoter operably linked to a fertility restorer polynucleotide encoding a fertility restorer; or
c. (a) and (b);
wherein expression of the sterility factor in the plant causes the plant to be sterile and wherein expression of the fertility restorer in the plant of (b) neutralizes, blocks, offsets, prevents, or inhibits the sterility factor causing the plant to be fertile.

2. The genetically modified plant of claim 1, wherein crossing a sterile plant to a fertility restorer plant produces fertile progeny plants.

3. The genetically modified plant of claim 1 or claim 2, wherein the sterility factor is barnase protein and wherein the fertility restorer is barstar protein.

4. The genetically modified plant of any of the preceding claims, wherein the sterile plant is male-sterile.

5. The genetically modified plant of any one of claims 2 to 4, wherein the sterility expression construct comprises a tapetum-specific promoter operably linked to a polynucleotide encoding barnase, wherein expression of the barnase in a stamen cell of the plant, kills or disables the stamen cell; wherein the fertility restorer expression construct comprises a tapetum-specific promoter operably linked to a polynucleotide encoding barstar; wherein crossing the sterile genetically modified plant to the fertility restorer genetically modified plant produces progeny comprising the sterility expression construct and the fertility restorer expression construct; and wherein said progeny are fertile due to neutralization, blocking, prevention, or inhibition of barnase activity by barstar.

6. The genetically modified plant of claim 5, wherein the tapetum-specific promoter comprises a nucleic acid sequence comprising at least about 75% or more, at least about 85% or more, at least about 95% or more, or 100% sequence identity with a nucleic acid sequence of SEQ ID NO: 38 or SEQ ID NO: 39.

7. The genetically modified plant of claim 5, wherein the barnase comprises an amino acid sequence comprising at least about 75% or more, at least about 85% or more, at least about 95% or more, or 100% sequence identity with an amino acid sequence of SEQ ID NO: 40.

8. The genetically modified plant of claim 5, wherein barstar comprises an amino acid sequence comprising at least about 75% or more, at least about 85% or more, at least about 95% or more, or 100% sequence identity with an amino acid sequence of SEQ ID NO: 41.

9. One or more expression constructs for generating a legume plant, plant part, or plant cell thereof, comprising a reversible sterility phenotype, the one or more expression constructs comprising:
a. a sterility expression construct comprising a promoter operably linked to a sterility polynucleotide encoding a sterility factor; and
b. a fertility restorer expression construct comprising a promoter operably linked to a fertility restorer polynucleotide encoding a fertility restorer.

10. The one or more expression constructs of claim 9, wherein the sterility factor is barnase protein and wherein the fertility restorer is barstar.

11. The one or more expression constructs of claim 10, wherein the sterility expression construct comprises a tapetum-specific promoter operably linked to a polynucleotide encoding barnase and wherein the fertility restorer expression construct comprises a tapetum-specific promoter operably linked to a polynucleotide encoding barstar.

12. The one or more expression constructs of claim 11, wherein the sterility expression construct comprises a nucleic acid sequence comprising at least about 75% or more, at least about 85% or more, at least about 95% or more, or 100% sequence identity with a nucleic acid sequence of SEQ ID NO: 42.

13. The one or more expression constructs of claim 12, wherein the fertility restorer expression construct comprises a nucleic acid sequence comprising at least about 75% or more, at least about 85% or more, at least about 95% or more, or 100% sequence identity with a nucleic acid sequence of SEQ ID NO: 43.

14. One or more genetically modified plants, plant cells, or parts thereof comprising one or more nucleic acid constructs of claims 9-13.

15. A genetically modified legume plant (Fabaceae), part thereof, or plant cell thereof, comprising an improved plant-pollinator floral phenotype, the genetically modified plant comprising a genetic modification of at least one target nucleic acid sequence in a polynucleotide encoding a floral trait, wherein the genetic modification modifies expression of the floral trait, wherein the modified expression of the floral trait improves a plant-pollinator floral phenotype when compared to a plant that does not comprise the genetic modification, wherein the improved plant-pollinator floral phenotype increases rate of cross-pollination of the genetically modified plant with a second plant when compared to the rate of cross-pollination between wild type plants.

16. The genetically modified plant of claim 15, wherein the floral trait is flower shape, flower size, flower color, flower odor, nectar composition, nectar volume, or any combination thereof.

17. The genetically modified plant of claim 15 or claim 16, wherein the floral trait is purple petal color, large petals, sweet nectar, or any combination thereof.

18. The genetically modified plant of any of the preceding claims, wherein the genetic modification modifies expression of a polynucleotide encoding a large petal phenotype.

19. The genetically modified plant of claim 18, wherein the polynucleotide encoding a large petal phenotype encodes an AP2-like ethylene-responsive transcription factor (ANT).

20. The genetically modified plant of claim 19, wherein the genetic modification increases expression of ANT.

21. The genetically modified plant of any one of claims 19 to 20, wherein the genetic modification comprises a polynucleotide encoding a programmable transcription regulation system comprising a programmable targeting domain and a transcription factor domain for transcriptional regulation of the polynucleotide encoding ANT.

22. The genetically modified plant of claim 21, wherein the programmable transcription regulation system comprises a CRISPR/Cas nuclease system modified for transcriptional regulation of a locus, wherein the CRISPR/Cas nuclease system comprises an engineered protein comprising a nuclease-deficient Cas9 protein (dCAS9 protein) fused to a transcriptional activator, and a guide RNA (gRNA).

23. The genetically modified plant of claim 22, wherein the engineered protein comprises dCas9 fused at its C terminus to a polypeptide comprising six copies of TALE TAD (Tail-TV CT engineered protein) fused at its C terminus to VP128 (dCas9-TV) and the gRNA comprises a sequence which binds to a polynucleotide sequence in a promoter region of the polynucleotide encoding ANT, to thereby target the engineered protein to the polynucleotide encoding ANT.

24. The genetically modified plant of claim 22 or claim 23, wherein the dCas9 protein comprises an amino acid sequence comprising at least about 75% or more, at least about 85% or more, at least about 95% or more, or 100% sequence identity with an amino acid sequence of SEQ ID NO: 24 and the polypeptide comprising six copies of TALE TAD (TAL) fused at its C terminus to VP128 (dCas9-TV) protein comprises at least about 75% or more, at least about 85% or more, at least about 95% or more, or 100% sequence identity with an amino acid sequence of SEQ ID NO: 48.

25. The genetically modified plant of any one of claims 21-24, wherein the programmable transcription regulation system is expressed in flower cells.

26. The genetically modified plant of any one of claims 23 to 25, wherein the polynucleotide encoding the programmable transcription regulation system comprises:
a. an expression construct for expressing dCas9-TV, wherein the expression construct comprises an AP3 promoter (pAP3; SEQ ID NO: 36) comprising a nucleic acid sequence comprising at least about 75% or more, at least about 85% or more, at least about 95% or more, or 100% sequence identity with a nucleic acid sequence of SEQ ID NO: 36, operably linked to a polynucleotide encoding dCas9-TV, wherein the polynucleotide encoding dCas9-TV comprises at least about 75% or more, at least about 85% or more, at least about 95% or more, or 100% sequence identity with a nucleic acid sequence of SEQ ID NO: 66; and
b. one or more expression constructs for expressing the gRNA wherein the one or more expression constructs comprise a pU10 promoter comprising a nucleic acid sequence comprising at least about 75% or more, at least about 85% or more, at least about 95% or more, or 100% sequence identity with a nucleic acid sequence of SEQ ID NO: 30, and a polynucleotide encoding a gRNA which binds to a polynucleotide sequence in a promoter region of the polynucleotide encoding ANT, wherein the polynucleotide encoding the gRNA comprises:
i. a nucleic acid sequence comprising at least about 75% or more, at least about 85% or more, at least about 95% or more, or 100% sequence identity with a nucleic acid sequence of SEQ ID NOs: 83;
ii. a nucleic acid sequence comprising at least about 75% or more, at least about 85% or more, at least about 95% or more, or 100% sequence identity with a nucleic acid sequence of SEQ ID NO: 76; or
iii. (i) and (ii).

27. The genetically modified plant of any one of claims 22 to 26, wherein
a. the gRNA comprises:
i. a nucleic acid sequence of SEQ ID NO: 1, SEQ ID NO: 2, SEQ ID NO: 3; or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 4;
ii. a nucleic acid sequence of SEQ ID NO: 5, SEQ ID NO: 6, or SEQ ID NO: 7, or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 8; or
iii. a nucleic acid sequence of any combination of SEQ ID NO: 1, SEQ ID NO: 2, SEQ ID NO: 3, SEQ ID NO: 5, SEQ ID NO: 6, and SEQ ID NO: 7, or any combination thereof, wherein the target is a nucleic acid sequence in a polynucleotide comprising at least about 75% or more, at least about 85% or more, at least about 95% or more, or 100% sequence identity with a nucleic acid sequence of SEQ ID NO: 4 or SEQ ID NO: 8.

28. The genetically modified plant of any one of claims 22 to 27, wherein the polynucleotide encoding the gRNA comprises:
a. an expression construct for expressing gRNA, wherein the expression construct comprises a pU10 promoter comprising a nucleic acid sequence comprising at least about 75% or more, at least about 85% or more, at least about 95% or more, or 100% sequence identity with a nucleic acid sequence of SEQ ID NO: 30, operably linked to a polynucleotide encoding the gRNA comprising SEQ ID NO: 1, SEQ ID NO: 2, SEQ ID NO: 3, wherein the expression construct encoding the gRNA comprises at least about 75% or more, at least about 85% or more, at least about 95% or more, or 100% sequence identity with a nucleic acid sequence of SEQ ID NO: 65;
b. an expression construct for expressing gRNA, wherein the expression construct comprises a pU10 promoter comprising a nucleic acid sequence comprising at least about 75% or more, at least about 85% or more, at least about 95% or more, or 100% sequence identity with a nucleic acid sequence of SEQ ID NO: 30, operably linked to a polynucleotide encoding the gRNA comprising SEQ ID NO: 5, SEQ ID NO: 6, or SEQ ID NO: 7, wherein the expression construct encoding the gRNA comprises at least about 75% or more, at least about 85% or more, at least about 95% or more, or 100% sequence identity with a nucleic acid sequence of SEQ ID NO: 67; or
c. (a) and (b).

29. The genetically modified plant of claim 17, wherein the polynucleotide encoding a large petal phenotype encodes a polynucleotide encoding a BIGPETALp (BPEp) protein.

30. The genetically modified plant of claim 29, wherein the genetic modification reduces expression of BPEp.

31. The genetically modified plant of claim 29 or claim 30, wherein the genetic modification comprises a polynucleotide encoding a programmable transcription regulation system comprising programmable transcription regulation system comprising a programmable targeting domain and a transcription repressor domain for reducing expression of the polynucleotide encoding BPEp.

32. The genetically modified plant of claim 31, wherein the programmable transcription regulation system comprises a CRISPR/Cas nuclease system modified for transcriptional regulation of a locus, wherein the CRISPR/Cas nuclease system comprises an engineered protein comprising a nuclease-deficient CAS9 protein (dCAS9) fused to a transcriptional repressor and a guide RNA (gRNA).

33. The genetically modified plant of claim 32, wherein the engineered protein comprises a dCAS9 protein fused at its C terminus to a polypeptide comprising an EAR-repression domain (SRDX; dCas9-SRDX) and the gRNA comprises a sequence which binds to a polynucleotide sequence in a promoter region of the polynucleotide encoding BPEp, to thereby target the engineered protein to the polynucleotide encoding BPEp.

34. The genetically modified plant of claim 32 or claim 33, wherein the dCas9 protein comprises an amino acid sequence comprising at least about 75% or more, at least about 85% or more, at least about 95% or more, or 100% sequence identity with an amino acid sequence of SEQ ID NO: 24 and the polypeptide comprising an EAR-repression domain comprises at least about 75% or more, at least about 85% or more, at least about 95% or more, or 100% sequence identity with an amino acid sequence of SEQ ID NO: 46.

35. The genetically modified plant of any one of claims 32 to 34, wherein the gRNA comprises a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 17, SEQ ID NO: 18, or both.

36. The genetically modified plant of any one of claims 32-35, wherein the programmable transcription regulation system is expressed in flower cells.

37. The genetically modified plant of any one of claims 32 to 36, wherein an expression construct encoding the programmable transcription regulation system comprises:
a. an expression construct for expressing dCas9-SRDX, wherein the expression construct comprises an AP3 promoter (pAP3) comprising a nucleic acid sequence comprising at least about 75% or more, at least about 85% or more, at least about 95% or more, or 100% sequence identity with a nucleic acid sequence of SEQ ID NO: 36, operably linked to a polynucleotide encoding dCas9-SRDX, wherein the polynucleotide encoding dCas9-SRDX comprises at least about 75% or more, at least about 85% or more, at least about 95% or more, or 100% sequence identity with a nucleic acid sequence of SEQ ID NO: 68; and
b. one or more expression constructs for expressing the gRNA wherein the one or more expression constructs comprise a pU10 promoter comprising a nucleic acid sequence comprising at least about 75% or more, at least about 85% or more, at least about 95% or more, or 100% sequence identity with a nucleic acid sequence of SEQ ID NO: 30, and a polynucleotide encoding a gRNA which binds to a polynucleotide sequence in a promoter region of the polynucleotide encoding BPEp, wherein the polynucleotide encoding the gRNA comprises a nucleic acid sequence comprising at least about 75% or more, at least about 85% or more, at least about 95% or more, or 100% sequence identity with a nucleic acid sequence of SEQ ID NO: 74.

38. The genetically modified plant of any of the preceding claims, wherein the genetic modification is in a polynucleotide encoding a sweet nectar phenotype.

39. The genetically modified plant of claim 38, wherein the polynucleotide encoding a sweet nectar phenotype encodes a sucrose-proton symporter 9 (SUC9) sucrose transporter protein.

40. The genetically modified plant of claim 39, wherein the genetic modification increases expression of SUC9.

41. The genetically modified plant of claim 40, wherein the genetic modification comprises a polynucleotide encoding a programmable transcription regulation system comprising a programmable transcription regulation system comprising a programmable targeting domain and a transcription factor domain for transcriptional regulation of the polynucleotide encoding SUC9.

42. The genetically modified plant of claim 41, wherein the programmable transcription regulation system comprises a CRISPR/Cas nuclease system modified for transcriptional regulation of a locus, wherein the CRISPR/Cas nuclease system comprises an engineered protein comprising a nuclease-deficient CAS9 protein (dCAS9) fused to a transcriptional activator and a guide RNA (gRNA).

43. The genetically modified plant of claim 42, wherein the engineered protein comprises dCAS9-TV and the gRNA comprises a sequence which binds to a polynucleotide sequence in a promoter region of a polynucleotide encoding SUC9, to thereby target the engineered protein to the polynucleotide encoding SUC9.

44. The genetically modified plant of any one of claims 41-43, wherein the programmable transcription regulation system is expressed in flower cells.

45. The genetically modified plant of any one of claims 41 to 44, wherein the gRNA comprises:
a. a nucleic acid sequence of SEQ ID NO: 20, SEQ ID NO: 21, SEQ ID NO: 22; or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 26;
b. a nucleic acid sequence of SEQ ID NO: 27, SEQ ID NO: 28, or SEQ ID NO: 29, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 33; or
c. any combination of SEQ ID NO: 20, SEQ ID NO: 21, SEQ ID NO: 22; SEQ ID NO: 27, SEQ ID NO: 28, or SEQ ID NO: 29.

46. The genetically modified plant of any one of claims 41 to 44, wherein an expression construct encoding the programmable transcription regulation system comprises:
a. an expression construct for expressing dCas9-TV, wherein the expression construct comprises an AGL1 promoter (pAGL1) comprising a nucleic acid sequence comprising at least about 75% or more, at least about 85% or more, at least about 95% or more, or 100% sequence identity with a nucleic acid sequence of SEQ ID NO: 19, operably linked to a polynucleotide encoding dCas9-TV, wherein the polynucleotide encoding dCas9-TV comprises at least about 75% or more, at least about 85% or more, at least about 95% or more, or 100% sequence identity with a nucleic acid sequence of SEQ ID NO: 66; and
b. one or more expression constructs for expressing the gRNA wherein the one or more expression constructs comprise a pU10 promoter comprising a nucleic acid sequence comprising at least about 75% or more, at least about 85% or more, at least about 95% or more, or 100% sequence identity with a nucleic acid sequence of SEQ ID NO: 30, and a polynucleotide encoding a gRNA which binds to a polynucleotide sequence in a promoter region of the polynucleotide encoding SUC9, wherein the polynucleotide encoding the gRNA comprises:
i. a nucleic acid sequence comprising at least about 75% or more, at least about 85% or more, at least about 95% or more, or 100% sequence identity with a nucleic acid sequence of SEQ ID NO: 77
ii. a nucleic acid sequence comprising at least about 75% or more, at least about 85% or more, at least about 95% or more, or 100% sequence identity with a nucleic acid sequence of SEQ ID NO: 78; or
iii. (i) and (ii).

47. The genetically modified plant of any of the preceding claims, wherein the genetic modification modifies expression of a polynucleotide encoding a purple petal phenotype.

48. The genetically modified plant of claim 47, wherein the polynucleotide encoding a purple petal phenotype encodes an allele of flower color gene flavonoid 3′5′-hydroxylase (W1) encoding a purple flower (w1) protein.

49. The genetically modified plant of claim 48, wherein the genetic modification comprises a nucleic acid expression construct comprising a white flower W1 promoter operably linked to a polynucleotide encoding the purple flower w1 protein.

50. The genetically modified plant of claim 49, wherein the white flower W1 promoter comprises a nucleic acid sequence comprising at least about 75% or more, at least about 85% or more, at least about 95% or more, or 100% sequence identity with a nucleic acid sequence of SEQ ID NO: 34, and wherein the polynucleotide encoding the w1 protein comprises at least about 75% or more, at least about 85% or more, at least about 95% or more, or 100% sequence identity with the nucleic acid sequence of SEQ ID NO: 35.

51. An engineered nucleic acid modification system for introducing in a plant a genetic modification in a polynucleotide encoding a plant-pollinator floral phenotype, the system comprising a nucleic acid expression construct comprising:
a. a programmable transcription regulation system targeted to a nucleic acid sequence in a polynucleotide encoding the plant-pollinator floral phenotype;
b. an expression construct comprising a promoter operably linked to a polynucleotide encoding a plant-pollinator floral phenotype operably linked to a promoter; or
c. (a) and (b),
wherein the programmable transcription regulation system, the expression construct, or both modify expression of a polynucleotide encoding a plant-pollinator floral phenotype, thereby increasing attraction of a pollinator to a plant, thereby resulting in an increased rate of cross-pollination.

52. The engineered nucleic acid modification system of claim 51, wherein the programmable transcription regulation system comprises a programmable targeting domain and a transcription factor domain transcriptional regulation of a locus.

53. The engineered nucleic acid modification system of claim 52, wherein the programmable transcription regulation system comprises a CRISPR/Cas nuclease system modified for transcriptional regulation of a locus, wherein the CRISPR/Cas nuclease system comprises an engineered protein comprising a nuclease-deficient CAS9 protein (dCAS9) fused to a transcriptional activator and a guide RNA (gRNA).

54. The engineered nucleic acid modification system of claim 53, wherein the engineered protein comprises dCas9 fused at its C terminus to a polypeptide comprising six copies of TALE TAD (TAL) fused at its C terminus to VP128 (dCas9-TV) and the gRNA comprises a sequence which binds to a polynucleotide sequence in a promoter region of a polynucleotide encoding ANT, to thereby target the engineered protein to the polynucleotide encoding ANT.

55. The engineered nucleic acid modification system of claim 54, wherein:
a. the dCas9-TV protein comprises an amino acid sequence comprising at least about 75% or more, at least about 85% or more, at least about 95% or more, or 100% sequence identity with an amino acid sequence of SEQ ID NO: 9; and
b. the gRNA comprises:
i. a nucleic acid sequence of SEQ ID NO: 1, SEQ ID NO: 2, SEQ ID NO: 3; or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 4;
ii. a nucleic acid sequence of SEQ ID NO: 5, SEQ ID NO: 6, SEQ ID NO: 7, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 8; or
iii. any combination of SEQ ID NO: 1, SEQ ID NO: 2, SEQ ID NO: 3, SEQ ID NO: 5, SEQ ID NO: 6, and SEQ ID NO: 7.

56. The engineered nucleic acid modification system of claim 53, wherein the engineered protein comprises dCAS9 fused at its C terminus to a polypeptide comprising an EAR-repression domain (SRDX; dCas9-SRDX) and the gRNA comprises a sequence which binds to a polynucleotide sequence in a promoter region of a polynucleotide encoding BPEp, to thereby target the engineered protein to the polynucleotide encoding BPEp.

57. The engineered nucleic acid modification system of claim 56, wherein:
c, wherein the dCas9-SRDX protein comprises an amino acid sequence comprising at least about 75% or more, at least about 85% or more, at least about 95% or more, or 100% sequence identity with an amino acid sequence of SEQ ID NO: 10; and
d. the gRNA comprises a nucleic acid sequence of SEQ ID NO: 11, SEQ ID NO: 12, SEQ ID NO: 13, SEQ ID NO: 14, SEQ ID NO: 15, SEQ ID NO: 16, or any combination thereof, and wherein the target is a nucleic acid sequence in a polynucleotide comprising at least about 75% or more, at least about 85% or more, at least about 95% or more, or 100% sequence identity with a nucleic acid sequence of SEQ ID NO: 17, SEQ ID NO: 18, or both.

58. The engineered nucleic acid modification system of claim 53, wherein the engineered protein comprises dCas9 fused at its C terminus to a polypeptide comprising six copies of TALE TAD (TAL) fused at its C terminus to VP128 (dCas9-TV) and the gRNA comprises a sequence which binds to a polynucleotide sequence in a promoter region of the polynucleotide encoding a sucrose-proton symporter 9 (SUC9) sucrose transporter, to thereby target the engineered protein to the polynucleotide encoding the SUC9 sucrose transporter.

59. The engineered nucleic acid modification system of claim 58, wherein:
a. the dCas9-TV protein comprises an amino acid sequence comprising at least about 75% or more, at least about 85% or more, at least about 95% or more, or 100% sequence identity with an amino acid sequence of SEQ ID NO: 9; and
b. the gRNA comprises:
iv. a nucleic acid sequence of SEQ ID NO: 20, SEQ ID NO: 21, SEQ ID NO: 22, or any combination thereof, and wherein the target is a nucleic acid sequence in a polynucleotide in a polynucleotide comprising at least about 75% or more, at least about 85% or more, at least about 95% or more, or 100% sequence identity with a nucleic acid sequence of SEQ ID NO: 26;
v. a nucleic acid sequence of SEQ ID NO: 27, SEQ ID NO: 28, or SEQ ID NO: 29, or any combination thereof and wherein the target is a nucleic acid sequence in a polynucleotide in a polynucleotide comprising at least about 75% or more, at least about 85% or more, at least about 95% or more, or 100% sequence identity with a nucleic acid sequence of SEQ ID NO: 33; or
vi. any combination of SEQ ID NO: 20, SEQ ID NO: 21, SEQ ID NO: 22; SEQ ID NO: 27, SEQ ID NO: 28, or SEQ ID NO: 29, or any combination thereof.

60. The engineered nucleic acid modification system of claim 51, wherein the genetic modification comprises a nucleic acid expression construct comprising a white flower W1 promoter operably linked to a polynucleotide encoding a purple flower W1 protein.

61. The engineered nucleic acid modification system of claim 60, wherein the W1 promoter comprises a nucleic acid sequence comprising at least about 75% or more, at least about 85% or more, at least about 95% or more, or 100% sequence identity with a nucleic acid sequence of SEQ ID NO: 34, and wherein the polynucleotide encoding the W1 protein comprises at least about 75% or more, at least about 85% or more, at least about 95% or more, or 100% sequence identity with the nucleic acid sequence of SEQ ID NO: 35.

62. One or more expression constructs for introducing a genetic modification into a legume plant (Fabaceae) of at least one target nucleic acid sequence in a polynucleotide encoding a floral trait, wherein the genetic modification modifies expression of a polynucleotide encoding a plant-pollinator floral phenotype, wherein the modified expression of the floral trait improves a plant-pollinator floral phenotype when compared to a plant that does not comprise the genetic modification, wherein the improved plant-pollinator floral phenotype increases rate of cross-pollination of the genetically modified plant with a second plant when compared to the rate of cross-pollination between wild type plants.

63. The one or more constructs of claim 62, wherein the genetic modification comprises a polynucleotide encoding a programmable transcription regulation system comprising a programmable targeting protein modified for transcriptional regulation of a locus.

64. The one or more constructs of claim 63, wherein the programmable transcription regulation system comprises a programmable targeting protein modified for transcriptional regulation of a polynucleotide encoding ANT.

65. The one or more constructs of claim 64, wherein the programmable transcription regulation system comprises a CRISPR/Cas nuclease system modified for transcriptional regulation of the polynucleotide encoding ANT, wherein the CRISPR/Cas nuclease system comprises an engineered protein comprising a nuclease-deficient CAS9 protein (dCAS9) fused at its C terminus to a polypeptide comprising six copies of TALE TAD (Tail-TV CT engineered protein) fused at its C terminus to VP128 (dCas9-TV), and a guide RNA (gRNA) comprising a sequence which binds to a polynucleotide sequence in a promoter region of the polynucleotide encoding ANT, to thereby target the engineered protein to the polynucleotide encoding ANT.

66. The one or more constructs of claim 65, wherein the expression construct for expressing dCas9-TV (pAP3::dCas9-TV) comprises an AP3 promoter operably linked to a polynucleotide encoding dCas9-TV, wherein the expression construct comprises a nucleic acid sequence comprising at least about 75% or more, at least about 85% or more, at least about 95% or more, or 100% sequence identity with a nucleic acid sequence of SEQ ID NO: 62.

67. The one or more constructs of claim 62 or claim 63, wherein the one or more expression constructs for expressing the gRNA comprises an pU10 promoter operably linked to a polynucleotide encoding the gRNA, and wherein the one or more constructs comprise one or more expression constructs for expressing the gRNA and wherein the one or more constructs comprise:
a. a nucleic acid sequence comprising at least about 75% or more, at least about 85% or more, at least about 95% or more, or 100% sequence identity with a nucleic acid sequence of SEQ ID NO: 65;
b. a nucleic acid sequence comprising at least about 75% or more, at least about 85% or more, at least about 95% or more, or 100% sequence identity with a nucleic acid sequence of SEQ ID NO: 67; or
c. (a) and (b).

68. The one or more constructs of claim 63, wherein the programmable transcription regulation system comprises a programmable targeting protein modified for transcriptional regulation of a polynucleotide encoding BPEp.

69. The one or more constructs of claim 68, wherein the programmable transcription regulation system comprises a CRISPR/Cas nuclease system modified for transcriptional regulation of the polynucleotide encoding BPEp, wherein the CRISPR/Cas nuclease system comprises an engineered protein comprising a nuclease-deficient CAS9 protein (dCAS9) fused at its C terminus to a polypeptide comprising an EAR-repression domain (SRDX; dCas9-SRDX), and a guide RNA (gRNA) comprising a sequence which binds to a polynucleotide sequence in a promoter region of the polynucleotide encoding BPEp, to thereby target the engineered protein to the polynucleotide encoding BPEp.

70. The one or more constructs of claim 69, wherein the expression construct for expressing dCas9-SRDX (pAP3::dCas9-SRDX) comprises an AP3 promoter operably linked to a polynucleotide encoding dCas9-SRDX, wherein the expression construct comprises a nucleic acid sequence comprising at least about 75% or more, at least about 85% or more, at least about 95% or more, or 100% sequence identity with a nucleic acid sequence of SEQ ID NO: 64.

71. The one or more constructs of claim 69, wherein the expression construct for expressing the gRNA comprises an pU10 promoter operably linked to a polynucleotide encoding the gRNA, and wherein the expression construct for expressing the gRNA comprises a nucleic acid sequence comprising at least about 75% or more, at least about 85% or more, at least about 95% or more, or 100% sequence identity with a nucleic acid sequence of SEQ ID NO: 70.

72. The one or more constructs of claim 63, wherein the programmable transcription regulation system comprises a programmable targeting protein modified for transcriptional regulation of a polynucleotide encoding SUC9.

73. The one or more constructs of claim 72, wherein the programmable transcription regulation system comprises a CRISPR/Cas nuclease system modified for transcriptional regulation of the polynucleotide encoding SUC9, wherein the CRISPR/Cas nuclease system comprises an engineered protein comprising a nuclease-deficient CAS9 protein (dCAS9) fused at its C terminus to a polypeptide comprising six copies of TALE TAD (Tail-TV CT engineered protein) fused at its C terminus to VP128 (dCas9-TV), and a guide RNA (gRNA) comprising a sequence which binds to a polynucleotide sequence in a promoter region of the polynucleotide encoding SUC9, to thereby target the engineered protein to the polynucleotide encoding SUC9.

74. The one or more constructs of claim 73, wherein the expression construct for expressing dCas9-TV (pAGL1::dCas9-TV) is under the control of a pAGL1 promoter comprises a nucleic acid sequence comprising at least about 75% or more, at least about 85% or more, at least about 95% or more, or 100% sequence identity with a nucleic acid sequence of SEQ ID NO: 63.

75. The one or more constructs of claim 73, wherein the one or more expression constructs for expressing the gRNA comprise a pU10 promoter operably linked to a polynucleotide encoding the gRNA, and wherein the comprise one or more constructs comprise:
a. a nucleic acid sequence comprising at least about 75% or more, at least about 85% or more, at least about 95% or more, or 100% sequence identity with a nucleic acid sequence of SEQ ID NO: 71;
b. a nucleic acid sequence comprising at least about 75% or more, at least about 85% or more, at least about 95% or more, or 100% sequence identity with a nucleic acid sequence of SEQ ID NO: 72; or
c. (a) and (b).

76. The one or more constructs of claim 62, wherein the introduced genetic modification comprises a nucleic acid expression construct comprising a white flower W1 promoter operably linked to a polynucleotide encoding a purple flower W1 protein

77. The one or more constructs of claim 76, wherein nucleic acid expression construct comprising a white flower W1 promoter operably linked to a polynucleotide encoding the purple flower W1 protein comprises a nucleic acid sequence comprising at least about 75% or more, at least about 85% or more, at least about 95% or more, or 100% sequence identity with a nucleic acid sequence of SEQ ID NO: 73.

78. One or more genetically modified plant, plant part, or cell thereof comprising one or more nucleic acid constructs of claims 62-77.

79. A genetically modified legume plant (Fabaceae), part thereof, or plant cell thereof, the genetically modified plant comprising a genetic modification in a polynucleotide encoding a floral trait selected from the group consisting of a purple petal phenotype, a large petal phenotype, a sweet nectar phenotype, and any combination thereof; wherein the genetic modification modifies expression of a floral trait, wherein the modified expression of the floral trait improves a plant-pollinator floral phenotype when compared to a plant that does not comprise the genetic modification, wherein the improved plant-pollinator floral phenotype increases rate of cross-pollination of the genetically modified plant with a second plant when compared to the rate of cross-pollination between wild type plants.

80. A genetically modified legume plant (Fabaceae), part thereof, or plant cell thereof, comprising an improved plant-pollinator floral phenotype and a reversible sterility phenotype, the genetically modified plant comprising:
a. an introduced genetic modification in a polynucleotide encoding a plant-pollinator floral phenotype selected from the group consisting of a purple petal phenotype, a large petal phenotype, a sweet nectar phenotype, and any combination thereof and a reversible sterility phenotype, wherein the plant further comprises:
i. a sterility expression construct comprising a promoter operably linked to a sterility polynucleotide encoding a sterility factor;
ii. a fertility restorer expression construct comprising a promoter operably linked to a fertility restorer polynucleotide encoding a fertility restorer; or
iii. (i) and (ii);
wherein the genetic modification of (a) increases rate of cross-pollination of the genetically modified plant with a second plant when compared to the rate of cross-pollination between wild type plants; wherein expression of the sterility factor in the plant causes the plant to be sterile and wherein expression of the fertility restorer in the plant of (i) or (iii) neutralizes, blocks, offsets, prevents, or inhibits the sterility factor causing the plant to be fertile; and wherein the combination of the improved plant-pollinator floral phenotype and the reversible sterility phenotype create a synergistic cross pollination phenotype.

81. A method of producing hybrid seed of a legume plant, the method comprising:
a. planting seeds of a sterile genetically modified plant of any one of claims 1-8 and seeds of a fertility restorer genetically modified plant of any one of claims 1-8;
b. allowing the seeds to germinate and grow into plants; and
c. allowing the fertility restorer plants to pollinate the sterile plants to thereby produce the hybrid seed on the sterile plant.

82. A method of producing hybrid seed of a legume plant, the method comprising:
d. planting seeds of genetically modified plants comprising an improved plant-pollinator floral phenotype; and
e. allowing the plants to pollinate to thereby produce the hybrid seed.

83. A method of synergistically producing hybrid seed of a legume plant, the method comprising:
a. planting seeds of a sterile genetically modified plant comprising reversible sterility phenotype and an improved plant-pollinator floral phenotype and seeds of a fertility restorer genetically modified plant comprising an improved plant-pollinator floral phenotype;
b. allowing the seeds to germinate and grow into plants; and
c. allowing the fertility restorer plants to pollinate the sterile plants to thereby produce the hybrid seed on the sterile plant;
wherein a combination of the improved plant-pollinator floral phenotype and the reversible sterility phenotype create a synergistic cross pollination phenotype.

84. A hybrid seed of a legume plant produced using a method of any one of claims 81 to 83.

85. A kit for generating a genetically modified legume plant comprising a reversible sterility phenotype, a genetically modified legume plant comprising an improved plant-pollinator phenotype, a genetically modified plant comprising a reversible sterility phenotype and an improved plant-pollinator phenotype, or for producing hybrid seed of the legume plant, the kit comprising:
a. one or more genetically modified plant of any one of claims 1-8 or any one of claims 15-50;
b. one or more expression constructs of any one of claims 9-13 or any one of claims 62-78:
c. one or more genetically modified plants, plant cells, or parts thereof claim 14 or claim 78;
d. hybrid seed of a legume plant of claim 79 or claim 80; or
e. any combination of (a) to (d).


SEQUENCE LISTING (109 sequences)

SEQ ID NO: 1 - DNA, 23 bp, Synthetic construct
  source 1..23: mol_type = other DNA
aaaaaatgtgaatgtggatgagg

SEQ ID NO: 2 - DNA, 23 bp, synthetic construct
  source 1..23: mol_type = other DNA
aaatttataacatttacatctgg

SEQ ID NO: 3 - DNA, 23 bp, synthetic construct
  source 1..23: mol_type = other DNA
tgatgtctttagaccagacaagg

SEQ ID NO: 4 - DNA, 1300 bp, Glycine max
  source 1..1300: mol_type = genomic DNA
atattataaaaaaaatattgttaataattaaagattaagatagatataatttaatataattgcaaattagatactattttatgtctatacttacaaggagatgaaaacagattaacacttgaattaattaatgtgatgtttcaatttaattaaatgtctcaaatttaaatttcgactccttaactacattaaatagttaagaaaacttttgtagaagatcttactcaatcaatacttgtgatgtctttagaccagacaaggagataaaaaaaaaagaaaaataagagattgatattttgataacgtaacatatataaaaaaaagagagaaatctaaagtgttgataaattgtttgtaattaataagcatccaaaaattattattatattttaaaagtcaattaatatgtgtatatgtatatttcaatttggaaataaaagtgatataatttagtttattgatccagatgtaaatgttataaatttttgatatcttattaaatttatatattttaaaaaaatgtgaatgtggatgaggtaaaagtataaaatataataacccaaaaatatatataaaatattaaataaacttttttggctgatttctgaatcttttcccaaatcccctcaccgtctatatcttggtcatttgcgctcactagattcacctactagcattcaagttttcagaaactgaccaaaaaaagttcatctcaccattaccattttgggctctttcttcttatccactttgaaaaattagattctgggtttcacatagggtttaagaaaaagctaagtctaaagaggggtggcagagttttctctctttttttttttataaaaaaataagagaaaaaaaaaaagcaaaagactttgtgtgtatatatacacataaatacacagatccctatggaatgaagtgggtgctcccaatatcacttgctagtaacacttattccttctgcttagtttaaagagaccagttttattatgctcgagtgaagaaagagagagagaaaagagagagtttggtttgcttgttttgttggtgtgagaaagaaaaaccttgctgtagctaaacaacaaaaaccaagtcttcattggtaacaagaagattattatttttatatgatttgtttatttatcacccaatgattgactttgcctagctgctgctgctacgagagaagatactgctggtggtggtgctagcaatagcaagtttaaagttcaaacctttttcaagtaatttataagttgagaaagaaaagaaaaaaccaagaaaaaaagaagcaaag

SEQ ID NO: 5 - DNA, 23 bp, synthetic construct
  source 1..23: mol_type = other DNA
ttttgtatctctctatatcttgg

SEQ ID NO: 6 - DNA, 23 bp, synthetic construct
  source 1..23: mol_type = other DNA
tttctttgtgtctcagctcaagg

SEQ ID NO: 7 - DNA, 23 bp, synthetic construct
  source 1..23: mol_type = other DNA
tgggaaaagattcagaactcagg

SEQ ID NO: 8 - DNA, 1346 bp, Glycine max
  source 1..1346: mol_type = genomic DNA
cttgcccgtcaaatctatggcccccttccactgcactgcttgcaaccttgtcaccgtaaaaacaaaaattaaattaataaaccccatttctgtggggtatatttctttttcaaaaacatttcatgtacaatcgttatactccaagatatagagagatacaaaaaaatataagatttaataacatttaaaatatgataagaaaagagaaacgagagatgttggtcagatatttaaaataagtatttttattttgatgatcaaaagttttaataatttgattattattattattattatttatttcttttcttatcatcctatattattaatgaaatcaattatttatttcttatttttttttctttgtgtctcagctcaaggattattaaaaaaaatatttttgtagtgtaaatataatatcatgaaaatataaaaagaatattaaataaacttttttggctacctgagttctgaatcttttcccaaatcccctcaccgtctatatcttggtcatttgctcactagattcacctactagcattcaagttttcagaaaatgaccaaaaaaaagttgatctcaccgttcccattttgggctctttcttcttatccactttgaaattagattctgggtttagcatagggtttaagaaaaagctaagtctagctggtgggggtggtttctcttttcttattttctttataaaaaaagaaaagaaagaaggcgaaagacttgtattgtgagtatatatgcacacacatacacagatccctatgaaatgaagtgggtgctcccagcatcacttgctagtaacacttattccttctgcttagtttaaagagaccagttttattatgctcgagtgaagaaagagagagaaagagaagaaagagttttggttggttgtttttcttgtgtgagaaagaaaaaagaaacaaaaaaaccttcctgtagctaaacaacaaaaaccaagtcttcattggcaacaagaagattattattttgatatgatttgtttatttgccacccaatgattgactttgcctagctgctgctacgagagacgatattctgctggtggtggtgctagcaatatgcatcggagatatttgtttttttttgcgttgttgttcaattagaattggagttttacttcgataatttataatacatatttaatatattgttgtaatgatacttcgattctgattgaaaaacaacaagaaaaaaagtacatgctaaactgcaattttttgttttagtgctttttttttcttgttttgcaatttgcaatattatggatctttaagagtgttgttcttgttgtggccttattgttgaagtg

SEQ ID NO: 9 - protein, 1842 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKPRGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGGGSGGDALDDFDLDMLGSDALDDFDLDMLGSDALDDFDLDMLGSDALDDFDLDMLARGSDALDDFDLDMLGSDALDDFDLDMLGSDALDDFDLDMLGSDALDDFDLDML

SEQ ID NO: 10 - protein, 1435 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGDGSGSGSGSGSVDLDLDLEELRLGFALDLDLELRLGFALDLDLELRLGFA

SEQ ID NO: 11 - DNA, 21 bp, synthetic construct
  source 1..21: mol_type = other DNA
tggagaatggtgcgcagtggg

SEQ ID NO: 12 - DNA, 23 bp, synthetic construct
  source 1..23: mol_type = other DNA
aagaggagtgacttggtttttgg

SEQ ID NO: 13 - DNA, 23 bp, synthetic construct
  source 1..23: mol_type = other DNA
actctctctacaagtggtaaagg

SEQ ID NO: 14 - DNA, 23 bp, synthetic construct
  source 1..23: mol_type = other DNA
cattagatgagtgatataaaagg

SEQ ID NO: 15 - DNA, 23 bp, synthetic construct
  source 1..23: mol_type = other DNA
agtttccgtgaatcaaaaatagg

SEQ ID NO: 16 - DNA, 23 bp, synthetic construct
  source 1..23: mol_type = other DNA
cacatgttctattgtaagattgg

SEQ ID NO: 17 - DNA, 934 bp, Glycine max
  source 1..934: mol_type = genomic DNA
cttttcgtaacccaaccgcacttgctccgagtctcagattccgaacacattaaaacccttcttacgtggaaccatcccattggccaaaccaaatcaacggtcaagatctcgtagagtatgagacagggtaatgcaatcgtacagcaacgaagaggggaccaccaaaacctcagtggaaacgacttcctacaatgccacctaggttcgtagaacaaaagtatatatatatatatatatatatatatatttttttccttctaaatcatggcattagaattactatgtaaaaattaaactctttccaaatatttaagataatttcacttacgtccatataacagtaatataagcgaagaaaaatataatattttttttttaaaaaatactcatacattttcttgtaatacttttaaaaatttgttaaaaattatcaatattatgaatctcactcttcttaatttaaatataagatccatcaaaataaataattttgatgaatcataaaaattaaaaagaacaaaaaaaaaaggttagaaatagtatgactagaattcttattttactttattaccgtattattattattattagtatgtaacattcatcaaacttgtcaaaagctaatttcttcatcagtagaaaaacacaactaatctctgattctttcagtcacctatttttgattcacggaaacttgaactcgaaaaattattaaataagaactcctttaccacttgtagagagagttatactataaagtataaacccactgcgcgcaccattctccactgtccaaggagatcaagcaccaaacccaacacacacctctctctctctctctctctctcttgtgctgttgttctatttctcattctttcgcaagtgaatgttgaactgactctgcggttcggttcggttttttttttttctgttt

SEQ ID NO: 18 - DNA, 689 bp, Glycine max
  source 1..689: mol_type = genomic DNA
ataattacaataatgatatcattattgtcatgaatctcttaaaataaattgatgataatttaatgatttagttatacaagtacataatattaaaattatttttgttttatcttaaaatcttagttataaataattttatttcacttaaacacattttattttaaaatttgtattaaaaaattaaacactaaaaaggatacaaccacctcaagtagactcttagaattctcatgttaaagtacttgtttgaagtaaagaagaattaaatttgataaaattaatttcataatgctaatatttaataaaaaatgcatataaattaatagtagtatcaattaccaatcttacaatagaacatgtgtacatcaattatccaataaaaaaagacttatagattttaaatgggttaaaaacttaaaatacattagatgagtgatataaaaggaagaaaagaaaaaagaggagtgacttggtttttgggagtcccgaggtgggtgtctctaaaacagtgagaaattgacagtaatggtggacagacaaatacagatactactattcttattctactactagtttccaacgaaaggagatcactatccaaaccattctcactttctcccttcaatgccactcttcttcctttctctttgagagactgcacacaaatttctactgtttccagagcgaca

SEQ ID NO: 19 - DNA, 2024 bp, synthetic construct
  source 1..2024: mol_type = other DNA
atatttgaatatacttaatgttgatgttaaatattatttcatccatcttaaaataagtgtcattctatttattttatatagacaaaaaaaagttaataaattaataaaaaaacagtaattttataaaactaaccttagaattaatatttcattaaaaaactaaaattacacttattagaaatattagaaaaaaaatatcactactaaaaatattattttttatgacgaacattctaaaatggttatatgaaatcattttagaatgtcatgcgatgacaattgtaaatacaagattgtttaacaaagacgattttacaaaaattgtcttaaaaattattgttttatgacggtttgatgaaaaccgtcttaaaatgattgtcattctaaaacgattttcgtaaaatcctcttaaaagttagtgcttttaatttttatgtctccctctcctctctcatttctctccctctcactctctactacttcttccgttccaaaataattgttgtattaggttattttacataggtgaagtaataaatagatgaaatataatgataattttagaaaattaatcttatatcatcatttaatacatttatatatagttcatcattaataaataaaaaaataattaatgttacatttaaaaactaaaatgataaaagtaattaatatacattaagaaactaaaatgacatatttttcttatacgacaattataacacgatgagggagtaatgaatattagttaaaattagtttttgatcacctaatttataattttgattcaatttagtcctttagttattttgggttcaatttaatcctctaatttttaaaattattttttttactttccattccatcacaagttccaaatgaatttgaatttaatcaacttatggtagtatatatagaactttgaaagattattatctattgtaaaaaaattcaatcttaagattttttttgcataacatgattcctaaactactcatcaggtgtcttaaagatcagttactagtgtaatggagctagctacttggctcacatttcaggtattttacgtaaaagtccttacatgcactgtcgccctaacactttgtttatgctaaccttgtcatcaaaatcaaaatgcagaaaagggaaaagaaagaaaaaattcaaactggctttcccatccccataaagacttgctaaatataaggagaagaagtgaagattatgatcagacatcacgtgggaacccttcttagttaaaattgtaataattcaagagagagggtcaagagtcactggaaactctcaggtttcaattttcaactgtacaaaataaaaattaaagagatgcaggggtcagttaaaggaaataaatcttgtagatataaatatctatatatagattatggttggataactttttatagaagaagaaaagaaaaacttgtcttataaatttaaatcatcttacacatgcatgcacttgaattttatagaaactcttataagctaaatatataaattaattttaatttatgaaaatatatagtttgcttcattttactatttatttacttttgttataagtgtaaacaaggccatattcgaagtaaaaggccaaaaactcaaaatccaagaagcaaaatggtttctttattttttttatgctttaaaaatttgaagcttcggaattttgcgtgcaaacttttatgtgcatgtccctctcattgaaattgaatcaaaccccacattaacaaattaaagtcactttcgaaaagaccctttgattatctccctctcctttttcccatacatgaagaaatggatggtttatggagcatgaaaggtgaacctcaagtctctctctctctcttctctctcatgcttaaattctccaacacgagagtaaattttagagtgagtggtaattgtggagggtctatttttctgtcataaatacctgtctgctaggccaacccacttccatattttgcatcctttgtacctcagttccaggttaacac

SEQ ID NO: 20 - DNA, 23 bp, synthetic construct
  source 1..23: mol_type = other DNA
tatcgtatagtaattagctgtgg

SEQ ID NO: 21 - DNA, 23 bp, synthetic construct
  source 1..23: mol_type = other DNA
catcatttacgtaaacgttttgg

SEQ ID NO: 22 - DNA, 23 bp, synthetic construct
  source 1..23: mol_type = other DNA
agggttgaagagtttatatatgg

SEQ ID NO: 23 - DNA, 4293 bp, synthetic construct
  source 1..4293: mol_type = other DNA
atggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagat

SEQ ID NO: 24 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 25 - DNA, 189 bp, synthetic construct
  source 1..189: mol_type = other DNA
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcag

SEQ ID NO: 26 - DNA, 786 bp, Glycine max
  source 1..786: mol_type = genomic DNA
ctctccctctgctgcgagtcaaataaagtgagttttcttgattaaatttgacaccataggccccaatcccatggttgccaattgctgtgcataaacgaacgaaggaaaaagaaacggagaaaaaggcaccttttcaccaaagtagtcgacacaaaatatacttcctctcttcttctcctaatacactccaaaattttgcctccctcaatcgtcattgcttctagaacatatacttcctatctctttatcccctgaaacaatcccaaattttctccatataatttagcctaggtttctaattcaagataaatcaaatccaactcaactattagaatcactatatatatatatatatatatattatccaaaacgtttacgtaaatgatgacatccacaaagattaaatttagtaaaatgaccacagtatgggttagaattaggatacaaaaaatgcagaaagtaaacccaacttcgccaatggataaacatagccattcataattatcaattagttatccttaaagaacacatgcaacgtaaaaaatgggtagtttttgccacagctaattactatacgatacaaatcctattattatcttaaattttgaaccactcatcccaatgttaaaagccatatataaactcttcaaccctctcacatttacacacactcctcctcctcctcctcctcgaggccttcacagagagaaagagaaaacatttcaaaaaaaataaaaaaaaataagcctctttctctctctgaatttctaagcctctctcacaaca

SEQ ID NO: 27 - DNA, 23 bp, synthetic construct
  source 1..23: mol_type = other DNA
tacgttgcatgtgttctttaagg

SEQ ID NO: 28 - DNA, 23 bp, synthetic construct
  source 1..23: mol_type = other DNA
ggagtgtgtgtaaatgtgagagg

SEQ ID NO: 29 - DNA, 23 bp, synthetic construct
  source 1..23: mol_type = other DNA
cctaattctaaccgatattgtgg

SEQ ID NO: 30 - DNA, 1111 bp, synthetic construct
  source 1..1111: mol_type = other DNA
gacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacc

SEQ ID NO: 31 - DNA, 2018 bp, synthetic construct
  source 1..2018: mol_type = other DNA
ttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaactt

SEQ ID NO: 32 - DNA, 90 bp, synthetic construct
  source 1..90: mol_type = other DNA
catctccactgacgtaagggatgacgcacaatcccactatccttcgcaagacccttcctctatataaggaagttcatttcatttggagag

SEQ ID NO: 33 - DNA, 634 bp, Glycine max
  source 1..634: mol_type = genomic DNA
tagtcgacacaaaatatacttcctctcttcttctcctaatacactccaaaattttgcctccctcaatcgtcattgcttctagaacatatacttcctatctctttatcccctgaaacaatcccaaattttctccatataatttagcctaggtttctaattcaagataaatcaaatccaactcaactattagaatcactatatatatatatattatccaaaacttttacgtaaatgatgacatccacaaagattaaatttagtaaaatgaccacaatatcggttagaattaggatacaaaaaatgcagaaagtaaacccaacttcgccaatggataaacatagccattcataattatcaattagttatccttaaagaacacatgcaacgtaaaaaatgggtagtttttgccaccattagctaattactatacgatacaaatcctattattatcttaaattttgaaccactcatcccaatgttaaaagtcatatataaactcttcaaccctctcacatttacacacactcctcctcctccttgaggctttcacagagagaaagagaaaacatttcaaaaaaaataaaaaaaaataagcctctttctctctctgaatttctaagcctctctcacaaca

SEQ ID NO: 34 - DNA, 2143 bp, Glycine max
  source 1..2143: mol_type = genomic DNA
cacatgtcacactctggtgcacatggtgttggtagaaagaccaaatgtcacgcccataactatcaatcgttgtcagtattgctatattccttggggatttacgtctcattgacaaccagagtggttttaagacaactgtttttatctagctatggaagattctcacgatgctcaagaatttgaagtgatcaatggggtttgatgcaagaggtaatgtcctgagttggagatttgagttattacgtctcgtataggtcattgaggattgaggagatcacaccgatatgatgcccgaggaggaggaagtgcatcatgagcttgtacgagatttagagccttcatcttaggaggaggattgattaccttcctgtgtttataccttaggcgtaattcaatacttttagtcgggtagtgatctgctctgaccctatatccttttttatatttgatgtatttgaggggtgagatcttcattgaaactgttttctttatgtaacagatgtgtatcgataatttaattacttttacactttgggttgtatccaaatttcttctattgtgacactatttatttgatttatttacatataaaggtttcatattataaatttgcgttcatttatttacatatttacttacgacgtgaaaacttgatgtttcttaagcaatgcatttattttattttatcttacttatacaaaaaaaatgatgagatatttttttaacttaaaaatagaaattaaaagttaatgacacgtattccacaatatttattattcaaaccattttatgtaatatttaaaaaaaaagtatatgagatgttacataaagagaccataagttggaatacctcccactaacaaaaagtaacaaataaattactaggattggccttaaaaaaatcgcgagttgttaattaatttactccttaatattataatagtcaattaattaaattggtccctaaaattaattgttttcttaaaaaaaataagtaaaacatgagaatccaaatcaattttagttggtggttggtttgagtatttttttttcatttttaaagaaaaataaaataaaagtgaaaatacattttcttaaaatttagaaaatatacataaaaacagatcaatagtaaagcaacgttttcaatcttttcttaagaagtaaaaatacggtgacactttgacattttttctcaatatattttttaaatcttaaaaatttaaaataaaaaatatttttaaaaaataaaaataaaaataaaaaataaacactcaaactaaaataagtaaaagttttaattttcacaatgtggcgcattaagactctcgaaatgctatttccaaaaagaatggtagctgactagatgcaatctctcgtaggcgttaggttggcaatggaaggtttggttgcacgcttgcacgccccatgtcagctgataacctactttcttagagtaacccactttccatattcacaaatgattaatggtcaatcttctaattcacgcactactcaccccaaccatccattaggatattccgagtctaatggctccattattaaaaaaaaaaaatctttttatttaaattttaacttttcatttttatatatttattcttaaacgaaaaatctaaactactatatttgattcatattctttatttattttttaaaatttccccattgtttaaattttttaaatcaaaatcaaaatcatttaagtttacttaattttaatattgtatgaaataattaaaattaaaaaaaaatgtttttttaacaaagtaaaaagaaattataatggtgatagaaccacgggaaagtatttgtgcaaaataattgaaattacaagtattattttctattacaatttttttaattgtttctttgcttttttttttttttaacttctattttgcaagcatgtattttatatttaatgtgtgtggagggggacggagtacaaactcttttttgtctatattcagtaaaatgttggtggagacgtgccttgcgtgatgaactaccatttcaaacaccacacgacaccacacatccattttaaatataaccccatcatagatatcccgaatcatcaaattattacttcatagcaactagcaaattaattagcttcacc

SEQ ID NO: 35 - DNA, 1774 bp, Glycine max
  source 1..1774: mol_type = other DNA
atggactcattgttacttctaaaagaaattgccacttccattttgatcttcttgatcactcgtctctccattcaaacattcctcaaaagctatcgccagaaactcccaccggggccaaaagggtggccagttgtgggtgcactccctctcatgggaagcatgcctcatgtcaccttagcaaagatggcaaaaaaatatggacctataatgtacctcaaaatgggcactaacaacatggttgtggcctctactccagctgctgctcgtgccttcctcaaaacccttgatcaaaacttttcaaaccggccctccaatgctggtgcaacccatttggcttatgatgcacgggatatggtgtttgctcattacggatcacggtggaagttgctaagaaaactaagtaacttgcacatgcttggaggaaaggcacttgatgattgggcccaaattcgagatgaagagatggggcacatgcttggtgcaatgtacgattgtaacaagagggatgaggctgtggtggtggcggagatgttgacatattcaatggccaacatgattggccaagttatattgagtcgtcgagtgtttgagacaaagggttcggagtctaacgagttcaaggacatggtggttgagctcatgaccgttgctggttacttcaacattggtgacttcataccctttttggccaagttggacttgcaaggcatagagcgtggcatgaagaagttgcacaagaagtttgatgcgttgttaacgagcatgattgaggagcatgttgcttctagtcacaagagaaagggcaagcccgatttcttagacatggtaatggctcatcatagtgagaactccgatggggaggaactatcgctcaccaacatcaaggcactactcttgaacctattcaccgcaggcaccgatacatcttcaagtataatagagtggtccttagccgagatgttgaagaagcccagcataatgaagaaggctcatgaagaaatggaccaagtcataggaagggatcgccgtctcaaagaatctgacataccaaagcttccctacttccaagccatttgcaaagagacctatagaaagcacccttcaacacccctaaacctgcctcgaatctcatctgaaccgtgccaagtgaatggttactacattcccgagaacactaggctgaatgtgaacatttgggccataggaagagaccctgatgtgtggaacaatcctttggagtttatgcccgagaggtttttgagtgggaagaatgccaaaattgacccacgtgggaatgattttgagcttattccatttggtgctgggaggaggatttgtgcagggactaggatggggattgtgttggttcactacattttgggcactttggtgcattcgtttgattggaagctacccaatggggtgagggagttagacatggaggagtcctttgggcttgccttgcaaaaaaaggttccacttgctgctttggttacccctaggttgaacccaagtgcttacatttcttagaattggttgggttcgaatattcaccagctatgttctctagccttattttgttgtccaatgattttgtggctgtggctacataaataagtaatgtttgggttgcacaacctatttgtatttgtaaggttctatgttacttggaaatccgttaccccaccacctgcaaggcttagatttttattcttcatggatctctaatttagctgtcttgttttggtttaattatatttttaattctcactca

SEQ ID NO: 36 - DNA, 2271 bp, Glycine max
  source 1..2271: mol_type = genomic DNA
tgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgtctctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagacgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgct

SEQ ID NO: 37 - DNA, 2123 bp, synthetic construct
  source 1..2123: mol_type = other DNA
caaacaacacatatagtgcagattcataaggtatagactttgttaatttgcaccctctcttaccctaaatcttgtgtctattgtattttatctctttccatatttgaatatacttaatgttgatgttaaatattatttcatccatcttaaaataagtgtcattctatttattttatatagacaaaaaaaagttaataaattaataaaaaaacagtaattttataaaactaaccttagaattaatatttcattaaaaaactaaaattacacttattagaaatattagaaaaaaaatatcactactaaaaatattattttttatgacgaacattctaaaatggttatatgaaatcattttagaatgtcatgcgatgacaattgtaaatacaagattgtttaacaaagacgattttacaaaaattgtcttaaaaattattgttttatgacggtttgatgaaaaccgtcttaaaatgattgtcattctaaaacgattttcgtaaaatcctcttaaaagttagtgcttttaatttttatgtctccctctcctctctcatttctctccctctcactctctactacttcttccgttccaaaataattgttgtattaggttattttacataggtgaagtaataaatagatgaaatataatgataattttagaaaattaatcttatatcatcatttaatacatttatatatagttcatcattaataaataaaaaaataattaatgttacatttaaaaactaaaatgataaaagtaattaatatacattaagaaactaaaatgacatatttttcttatacgacaattataacacgatgagggagtaatgaatattagttaaaattagtttttgatcacctaatttataattttgattcaatttagtcctttagttattttgggttcaatttaatcctctaatttttaaaattattttttttactttccattccatcacaagttccaaatgaatttgaatttaatcaacttatggtagtatatatagaactttgaaagattattatctattgtaaaaaaattcaatcttaagattttttttgcataacatgattcctaaactactcatcaggtgtcttaaagatcagttactagtgtaatggagctagctacttggctcacatttcaggtattttacgtaaaagtccttacatgcactgtcgccctaacactttgtttatgctaaccttgtcatcaaaatcaaaatgcagaaaagggaaaagaaagaaaaaattcaaactggctttcccatccccataaagacttgctaaatataaggagaagaagtgaagattatgatcagacatcacgtgggaacccttcttagttaaaattgtaataattcaagagagagggtcaagagtcactggaaactctcaggtttcaattttcaactgtacaaaataaaaattaaagagatgcaggggtcagttaaaggaaataaatcttgtagatataaatatctatatatagattatggttggataactttttatagaagaagaaaagaaaaacttgtcttataaatttaaatcatcttacacatgcatgcacttgaattttatagaaactcttataagctaaatatataaattaattttaatttatgaaaatatatagtttgcttcattttactatttatttacttttgttataagtgtaaacaaggccatattcgaagtaaaaggccaaaaactcaaaatccaagaagcaaaatggtttctttattttttttatgctttaaaaatttgaagcttcggaattttgcgtgcaaacttttatgtgcatgtccctctcattgaaattgaatcaaaccccacattaacaaattaaagtcactttcgaaaagaccctttgattatctccctctcctttttcccatacatgaagaaatggatggtttatggagcatgaaaggtgaacctcaagtctctctctctctcttctctctcatgcttaaattctccaacacgagagtaaattttagagtgagtggtaattgtggagggtctatttttctgtcataaatacctgtctgctaggccaacccacttccatattttgcatcctttgtacctcagttccaggttaacac

SEQ ID NO: 38 - DNA, 1567 bp, synthetic construct
  source 1..1567: mol_type = other DNA
gaattcgtttccatagcctaaggccacgagtgcacgtgttcataggctagttgtgttaaccttggggagcagtcgacaatttggattgcatcgaggtcttgccaaattttactttcaagacagtgatttcgacggcacaataaacttccaattagttatcttctcttttgttctgtattgtgtttttttctatttttctaacaatggaaatagtatataccggccaacagtgttgcactgttgtctggaccttgtcaaaggttcgacagtgcatagtttggtctcgatgtggcatatgggggttctgataagtgatgagattatgaaaataatttaattttagctattaaaaatacaatacaactttcaaaagcgggaaacttgtttgattatgatctaggattctaggtctttttttttaagttcgagacagaaaaccaaaaacaaaacagctaataagaagaagaaaaacaatacagcaatattcccacaaaaataaatttgttcttttgcgtactagccataggatttaattatatatatgggccagtccagaagcattgtcactgatagatcatagactagtaaaaaagaatgaaccacttcagaacactaactatatatataaagatagattcatatctatttagatcgtgattattaatacaagaaaatatttgtatttcttaattttttgtttttaaatttatgtaaatatattgacatcttttttaacgaaaattcaaattttaattcattatattacaagaaattaacacattttactagaccaactctcatgttacaacactatatatgttgaacattacaagaatgccactttatctcacccctttaattaggaaaagcaaaaatctatttgcatataattttgtaccaaaaaattatacaataaaaaagaaaaagagaaaagtaaaagataagacgactgatgtaataaaaagagagaaaaaataatactaaaaaataatataaataacataactcttaaaaaaaaaaggtaacactgttgagtattttttttattacttttctctaaaaaggtatcatggtttcactaatgtccaggttttttttttccaaataaactcctttccggtggtaggtatcgtttcacacttgaatacttttttgcaatatttttcgtaacactttatttctctccattacataattttacccttgcctatgtctttattctgccacatataccctaagagtgagcaaatgtgtgcacgcatataacctgattggactatatatcggacttgatacaaatgtgtgctcacatgaagagaaaaaggattgaacccatgatcatcatcatgtcacatgcagatgccatgcattcaacaccaaagttatgatcagaaaacctctcaaggtaggtgactcaccaaatgaacaagccaattttttgcttcccaaatttcaccataaatagggacactactccatccaaaccatacacgaccacatacagtgggattagtaagtaaacaaattaattaataaccaaaagttataatttcaattgaatttagtc

SEQ ID NO: 39 - DNA, 1545 bp, synthetic construct
  source 1..1545: mol_type = other DNA
tttgtaatattatgatttacgtaaaaatctatctaatgattttatttaaactatagttttgaacttttccaactgaaatttcaacacagtaattatggttgcctgtaactcatattttgcaagccccacttcaattatgcagcctaacccaaaagtggggatatgtgatttcaaaggatatatgcatcaatcatttctccaaattaggaaagtaatttatataaaattaacttgactttaagttagaactgcattttactgaataaaagttataattattaataaataaatggttaagattttaacgttataatatattcatacagtacaaattcaagtttttattcttgaaataaaatttttattttaggtatttcatttttatgcagtcatgtgtcttttttttatttttgaaacatattttatggtcatttatatcaattcaaattagaattttcttcctctctaactaattattcaacaacaaaaaaaaactattttttcaagtttttattttactcagtgttttaatcaatattaagcataatgagtatttataaatcattctcaaaataatattttacatgttaaataatttttaagtatttttaagagtgtgtttaattgtgttttaatttcttagtctattttttaaaaactattttatgaaacatttttcataaactgatgtttagagagtaaaagtggaaaagtaagatgtgaaaatacaaggaagaacatgaataatagaaaattaaaagtaaaaaacccaaacatcgatcatagagatgttatagttttttggttttgaaaaaactaaaaaaagagatagtagttttaaaaacagttttcgaaactatttttatcaaacaacataatttttttagcttttaaataaaaacagctttaaaaaatgtccaggtttttttccaaataaattcaattccatattttaataagtaatggttaatccagtggtaggtatatattgtttaacacttgaaatttttttccattcaatttttttaatatttttcataacacttaatttatttattacatatatcatatgttatattattatttttctctattctcttcctttctctctagttatttgtaaaaaaaaaaacatataatttatttccattgcataaattttatccatgtctatccatgcctatgtttttattctgccacatataccctaagtgttagcaaatatgtgcacacattaacctaattaaactatactagaactatatatcggacttgatagaaagtggagataagccaaggaagacaagaaaaaggattgaacacatgatcatcctcatgtcacatgcagttgccatggatccaacaccaaagttatgatcagaaaacctctcaaggcaggtgactcacgaaatgaacaagccaatttgcttccaaaattcaccataaatagggacactactcaatgcaaaccatgcatacacaatcatatacagtgggattagtaaacaaattaattaataaccaaaagttatttaaattgaagtc

SEQ ID NO: 40 - protein, 157 aa, synthetic construct
MMKMEGIALKKRLSWISVCLLVLVSAAGMLFSTAAKTETSSHKAHTEAQVINTFDGVADYLQTYHKLPDNYITKSEAQALGWVASKGNLADVAPGKSIGGDIFSNREGKLPGKSGRTWREADINYTSGFRNSDRILYSSDWLIYKTTDHYQTFTKIR

SEQ ID NO: 41 - protein, 90 aa, synthetic construct
MKKAVINGEQIRSISDLHQTLKKELALPEYYGENLDALWDCLTGWVEYPLVLEWRQFEQSKQLTENGAESVLQVFREAKAEGCDITIILS

SEQ ID NO: 42 - DNA, 2037 bp, synthetic construct
  source 1..2037: mol_type = other DNA
tttgtaatattatgatttacgtaaaaatctatctaatgattttatttaaactatagttttgaacttttccaactgaaatttcaacacagtaattatggttgcctgtaactcatattttgcaagccccacttcaattatgcagcctaacccaaaagtggggatatgtgatttcaaaggatatatgcatcaatcatttctccaaattaggaaagtaatttatataaaattaacttgactttaagttagaactgcattttactgaataaaagttataattattaataaataaatggttaagattttaacgttataatatattcatacagtacaaattcaagtttttattcttgaaataaaatttttattttaggtatttcatttttatgcagtcatgtgtcttttttttatttttgaaacatattttatggtcatttatatcaattcaaattagaattttcttcctctctaactaattattcaacaacaaaaaaaaactattttttcaagtttttattttactcagtgttttaatcaatattaagcataatgagtatttataaatcattctcaaaataatattttacatgttaaataatttttaagtatttttaagagtgtgtttaattgtgttttaatttcttagtctattttttaaaaactattttatgaaacatttttcataaactgatgtttagagagtaaaagtggaaaagtaagatgtgaaaatacaaggaagaacatgaataatagaaaattaaaagtaaaaaacccaaacatcgatcatagagatgttatagttttttggttttgaaaaaactaaaaaaagagatagtagttttaaaaacagttttcgaaactatttttatcaaacaacataatttttttagcttttaaataaaaacagctttaaaaaatgtccaggtttttttccaaataaattcaattccatattttaataagtaatggttaatccagtggtaggtatatattgtttaacacttgaaatttttttccattcaatttttttaatatttttcataacacttaatttatttattacatatatcatatgttatattattatttttctctattctcttcctttctctctagttatttgtaaaaaaaaaaacatataatttatttccattgcataaattttatccatgtctatccatgcctatgtttttattctgccacatataccctaagtgttagcaaatatgtgcacacattaacctaattaaactatactagaactatatatcggacttgatagaaagtggagataagccaaggaagacaagaaaaaggattgaacacatgatcatcctcatgtcacatgcagttgccatggatccaacaccaaagttatgatcagaaaacctctcaaggcaggtgactcacgaaatgaacaagccaatttgcttccaaaattcaccataaatagggacactactcaatgcaaaccatgcatacacaatcatatacagtgggattagtaaacaaattaattaataaccaaaagttatttaaattgaagtctaatttcaattgaatttagtcatgatgaaaatggagggcatagctttgaagaagcgtctgtcttggattagcgtatgtttgctcgtattggtgtctgccgcaggaatgctgttcagcactgctgctaaaacagagacctcatcccataaagctcatactgaggctcaagtgataaataccttcgatggggtcgctgactaccttcagacctaccacaaattgcccgacaattacattaccaagagcgaggcccaagctctggggtgggttgcctctaagggaaatctcgcagatgtggcccctggaaaatcaattggcggcgacatattttctaacagggaaggcaaactgcccggtaagagtgggcgtacatggcgtgaagcagatattaactatacatcaggctttagaaatagtgaccgtattctttattcttcagactggttgatttacaagaccactgatcattatcaaacattcacaaagatccgt

SEQ ID NO: 43 - DNA, 1837 bp, synthetic construct
  source 1..1837: mol_type = other DNA
gaattcgtttccatagcctaaggccacgagtgcacgtgttcataggctagttgtgttaaccttggggagcagtcgacaatttggattgcatcgaggtcttgccaaattttactttcaagacagtgatttcgacggcacaataaacttccaattagttatcttctcttttgttctgtattgtgtttttttctatttttctaacaatggaaatagtatataccggccaacagtgttgcactgttgtctggaccttgtcaaaggttcgacagtgcatagtttggtctcgatgtggcatatgggggttctgataagtgatgagattatgaaaataatttaattttagctattaaaaatacaatacaactttcaaaagcgggaaacttgtttgattatgatctaggattctaggtctttttttttaagttcgagacagaaaaccaaaaacaaaacagctaataagaagaagaaaaacaatacagcaatattcccacaaaaataaatttgttcttttgcgtactagccataggatttaattatatatatgggccagtccagaagcattgtcactgatagatcatagactagtaaaaaagaatgaaccacttcagaacactaactatatatataaagatagattcatatctatttagatcgtgattattaatacaagaaaatatttgtatttcttaattttttgtttttaaatttatgtaaatatattgacatcttttttaacgaaaattcaaattttaattcattatattacaagaaattaacacattttactagaccaactctcatgttacaacactatatatgttgaacattacaagaatgccactttatctcacccctttaattaggaaaagcaaaaatctatttgcatataattttgtaccaaaaaattatacaataaaaaagaaaaagagaaaagtaaaagataagacgactgatgtaataaaaagagagaaaaaataatactaaaaaataatataaataacataactcttaaaaaaaaaaggtaacactgttgagtattttttttattacttttctctaaaaaggtatcatggtttcactaatgtccaggttttttttttccaaataaactcctttccggtggtaggtatcgtttcacacttgaatacttttttgcaatatttttcgtaacactttatttctctccattacataattttacccttgcctatgtctttattctgccacatataccctaagagtgagcaaatgtgtgcacgcatataacctgattggactatatatcggacttgatacaaatgtgtgctcacatgaagagaaaaaggattgaacccatgatcatcatcatgtcacatgcagatgccatgcattcaacaccaaagttatgatcagaaaacctctcaaggtaggtgactcaccaaatgaacaagccaattttttgcttcccaaatttcaccataaatagggacactactccatccaaaccatacacgaccacatacagtgggattagtaagtaaacaaattaattaataaccaaaagttataatttcaattgaatttagtcatgaaaaaagctgtaataaacggtgaacaaatacgcagcatatctgacctccaccagactctcaagaaagagcttgctttgcccgagtattatggtgagaatctggacgcattgtgggactgcctcacaggatgggtggagtacccattggtcttggagtggcgtcagtttgaacagagcaaacagttgactgagaacggtgccgagtccgttcttcaagtatttcgcgaggccaaggccgaaggctgcgacataaccatcatcctctct

SEQ ID NO: 44 - DNA, 1815 bp, synthetic construct
  source 1..1815: mol_type = other DNA
tttgtaatattatgatttacgtaaaaatctatctaatgattttatttaaactatagttttgaacttttccaactgaaatttcaacacagtaattatggttgcctgtaactcatattttgcaagccccacttcaattatgcagcctaacccaaaagtggggatatgtgatttcaaaggatatatgcatcaatcatttctccaaattaggaaagtaatttatataaaattaacttgactttaagttagaactgcattttactgaataaaagttataattattaataaataaatggttaagattttaacgttataatatattcatacagtacaaattcaagtttttattcttgaaataaaatttttattttaggtatttcatttttatgcagtcatgtgtcttttttttatttttgaaacatattttatggtcatttatatcaattcaaattagaattttcttcctctctaactaattattcaacaacaaaaaaaaactattttttcaagtttttattttactcagtgttttaatcaatattaagcataatgagtatttataaatcattctcaaaataatattttacatgttaaataatttttaagtatttttaagagtgtgtttaattgtgttttaatttcttagtctattttttaaaaactattttatgaaacatttttcataaactgatgtttagagagtaaaagtggaaaagtaagatgtgaaaatacaaggaagaacatgaataatagaaaattaaaagtaaaaaacccaaacatcgatcatagagatgttatagttttttggttttgaaaaaactaaaaaaagagatagtagttttaaaaacagttttcgaaactatttttatcaaacaacataatttttttagcttttaaataaaaacagctttaaaaaatgtccaggtttttttccaaataaattcaattccatattttaataagtaatggttaatccagtggtaggtatatattgtttaacacttgaaatttttttccattcaatttttttaatatttttcataacacttaatttatttattacatatatcatatgttatattattatttttctctattctcttcctttctctctagttatttgtaaaaaaaaaaacatataatttatttccattgcataaattttatccatgtctatccatgcctatgtttttattctgccacatataccctaagtgttagcaaatatgtgcacacattaacctaattaaactatactagaactatatatcggacttgatagaaagtggagataagccaaggaagacaagaaaaaggattgaacacatgatcatcctcatgtcacatgcagttgccatggatccaacaccaaagttatgatcagaaaacctctcaaggcaggtgactcacgaaatgaacaagccaatttgcttccaaaattcaccataaatagggacactactcaatgcaaaccatgcatacacaatcatatacagtgggattagtaaacaaattaattaataaccaaaagttatttaaattgaagtcatgaaaaaagctgtaataaacggtgaacaaatacgcagcatatctgacctccaccagactctcaagaaagagcttgctttgcccgagtattatggtgagaatctggacgcattgtgggactgcctcacaggatgggtggagtacccattggtcttggagtggcgtcagtttgaacagagcaaacagttgactgagaacggtgccgagtccgttcttcaagtatttcgcgaggccaaggccgaaggctgcgacataaccatcatcctctct

SEQ ID NO: 45 - DNA, 201 bp, synthetic construct
  source 1..201: mol_type = other DNA
aagcgcccagccgctaccaaaaaagctggacaggctaaaaaaaagaagggtgacggcagcgggtccggctccgggtccggttccgtcgatctcgatttggatctggaacttcgcctggggttcgcactcgatctcgaccttgaactgcgtcttggctttgctctcgatcttgatctggagttgaggcttggattcgcttag

SEQ ID NO: 46 - protein, 67 aa, synthetic construct
KRPAATKKAGQAKKKKGDGSGSGSGSGSVDLDLDLEELRLGFALDLDLELRLGFALDLDLELRLGFA

SEQ ID NO: 47 - DNA, 1425 bp, synthetic construct
  source 1..1425: mol_type = other DNA
aagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctga

SEQ ID NO: 48 - protein, 474 aa, synthetic construct
KRPAATKKAGQAKKKKPRGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGGGSGGDALDDFDLDMLGSDALDDFDLDMLGSDALDDFDLDMLGSDALDDFDLDMLARGSDALDDFDLDMLGSDALDDFDLDMLGSDALDDFDLDMLGSDALDDFDLDML

SEQ ID NO: 49 - DNA, 1428 bp, synthetic construct
  source 1..1428: mol_type = other DNA
atggtgtccaagggcgaagaggtgattaaagagttcatgaggttcaaagtgagaatggagggcagtatgaacggccatgagttcgagatcgaaggtgaaggcgaaggtagaccatacgagggcacccagacggcgaaactgaaagtgacgaagggcggcccactgcccttcgcctgggacattctgagtccacaatttatgtacggttccaaggcatacgttaagcaccccgccgacatccctgattacaaaaagctgtcgttcccggaggggtttaaatgggaaagagtgatgaattttgaggacggagggctcgtcactgtgactcaagactcctcacttcaagacggcaccctaatttacaaggtgaagatgaggggaacaaatttcccccctgatggccctgttatgcagaaaaaaacaatgggatgggaggcatccaccgagagactttacccgcgcgacggagtgctcaaaggcgaaatccatcaggcactgaaactaaaggacggcggccactacctggttgagttcaaaactatatatatggccaagaaacccgtccagttgccgggatattactatgtggacacaaaactggatataacgtcccacaacgaagattacaccatagtggaacagtatgagcgcagtgagggtcgtcaccatctattcctcggacatggcacgggttccacgggctcgggttccagcggcacggcaagctcggaggacaacaacatggcggtgatcaaagagttcatgcggtttaaagtccgtatggagggcagcatgaatgggcacgaatttgaaattgagggagaaggcgagggccgaccatatgaggggacacaaaccgcgaaactaaaagtcaccaaaggtgggccgcttccttttgcctgggacattctctctcctcagttcatgtatggctccaaggcctacgttaaacatccggctgatatccccgattataagaaattgtctttcccggaggggtttaaatgggagcgggttatgaatttcgaggatggtggactggttactgttacacaagattcgagtctgcaggatggcacattgatatacaaagtcaaaatgaggggtacgaattttcccccggacggcccggtgatgcagaaaaaaactatgggctgggaggccagcacggagaggctgtacccgagggacggggttttgaaaggggagatacaccaggcgcttaaactgaaggacggggggcactatctggtggaatttaagacgatatatatggcgaaaaagcccgtgcagctacctggctactactacgtcgataccaaactcgatattacctctcataacgaggactacactatcgtggagcaatatgaaagatcagaaggacgtcaccacctttttctatacggaatggacgagctttacaaa

SEQ ID NO: 50 - protein, 476 aa, synthetic construct
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 51 - DNA, 693 bp, synthetic construct
  source 1..693: mol_type = other DNA
atggctcagtccaagcacggccttaccaaggagatgaccatgaagtacaggatggagggctgcgtggatggccacaagttcgtgatcaccggcgagggcatcggctacccattcaagggcaaacaggctatcaacctttgcgtggtggagggcggcccacttccattcgctgaggatatcctttccgctgctttcatgtacggcaacagggtgttcaccgagtacccacaggatatcgctgattacttcaagaactcctgcccagctggctacacctgggataggtccttccttttcgaggatggcgctgtgtgcatctgcaacgctgatatcaccgtgtccgtggaggagaactgcatgtaccacgagtccaagttctacggcgtgaacttcccagctgatggcccagtgatgaagaagatgaccgataactgggagccatcctgcgagaagatcatcccagtgccaaagcagggcatccttaagggcgatgtgtccatgtaccttcttcttaaggatggcggcaggcttaggtgccagttcgataccgtgtacaaggctaagtccgtgccaaggaagatgccagattggcacttcatccagcacaagcttaccagggaggataggtccgatgctaagaaccagaagtggcaccttaccgagcacgctatcgcttccggctccgcccttcca

SEQ ID NO: 52 - protein, 231 aa, synthetic construct
MAQSKHGLTKEMTMKYRMEGCVDGHKFVITGEGIGYPFKGKQAINLCVVEGGPLPFAEDILSAAFMYGNRVFTEYPQDIADYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 53 - DNA, 2015 bp, synthetic construct
  source 1..2015: mol_type = other DNA
atgggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggtaagtcttactctctcttttttggtctgtatttttaattttttgaagtatactatttgtactgacgctaataatcttttttcaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtacttcatgcttcaacgtgtaacttaagagatactgtgtgaaattttatatttccatacatttgcttgacctttgctttttgtcaatttttttccccttacaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagg

SEQ ID NO: 54 - DNA, 1826 bp, synthetic construct
  source 1..1826: mol_type = other DNA
atgggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagg

SEQ ID NO: 55 - protein, 608 aa, synthetic construct
MGQSLMLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 56 - DNA, 24 bp, synthetic construct
  source 1..24: mol_type = other DNA
ccaaagaagaagcggaaggtctaa

SEQ ID NO: 57 - protein, 7 aa, synthetic construct
PKKKRKV

SEQ ID NO: 58 - DNA, 16501 bp, synthetic construct
  source 1..16501: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgacaagcttagaccttccgcttcttctttggcgaacttggaagggcggagccggaagcgatagcgtgctcggtaaggtgccacttctggttcttagcatcggacctatcctccctggtaagcttgtgctggatgaagtgccaatctggcatcttccttggcacggacttagccttgtacacggtatcgaactggcacctaagcctgccgccatccttaagaagaaggtacatggacacatcgcccttaaggatgccctgctttggcactgggatgatcttctcgcaggatggctcccagttatcggtcatcttcttcatcactgggccatcagctgggaagttcacgccgtagaacttggactcgtggtacatgcagttctcctccacggacacggtgatatcagcgttgcagatgcacacagcgccatcctcgaaaaggaaggacctatcccaggtgtagccagctgggcaggagttcttgaagtaatcagcgatatcctgtgggtactcggtgaacaccctgttgccgtacatgaaagcagcggaaaggatatcctcagcgaatggaagtgggccgccctccaccacgcaaaggttgatagcctgtttgcccttgaatgggtagccgatgccctcgccggtgatcacgaacttgtggccatccacgcagccctccatcctgtacttcatggtcatctccttggtaaggccgtgcttggactgagccattgacttcaatttaaataacttttggttattaattaatttgtttactaatcccactgtatatgattgtgtatgcatggtttgcattgagtagtgtccctatttatggtgaattttggaagcaaattggcttgttcatttcgtgagtcacctgccttgagaggttttctgatcataactttggtgttggatccatggcaactgcatgtgacatgaggatgatcatgtgttcaatcctttttcttgtctaccttggcttatctccactttctatcaagtccgatatatagttctagtatagtttaattaggttaatgtgtgcacatatttgctaacacttagggtatatgtggcagaataaaaacataggcatggatagacatggataaaatttatgcaatggaaataaattatatgtttttttttttacaaataactagagagaaaggaagagaatagagaaaaataataatataacatatgatatatgtaataaataaattaagtgttatgaaaaatattaaaaaaattgaatggaaaaaaatttcaagtgttaaacaatatatacctaccactggattaaccattacttattaaaatatggaattgaatttatttggaaaaaaacctggacattttttaaagctgtttttatttaaaagctaaaaaaattatgttgtttgataaaaatagtttcgaaaactgtttttaaaactactatctctttttttagttttttcaaaaccaaaaaactataacatctctatgatcgatgtttgggttttttacttttaattttctattattcatgttcttccttgtattttcacatcttacttttccacttttactctctaaacatcagtttatgaaaaatgtttcataaaatagtttttaaaaaatagactaagaaattaaaacacaattaaacacactcttaaaaatacttaaaaattatttaacatgtaaaatattattttgagaatgatttataaatactcattatgcttaatattgattaaaacactgagtaaaataaaaacttgaaaaaatagtttttttttgttgttgaataattagttagagaggaagaaaattctaatttgaattgatataaatgaccataaaatatgtttcaaaaataaaaaaaagacacatgactgcataaaaatgaaatacctaaaataaaaattttatttcaagaataaaaacttgaatttgtactgtatgaatatattataacgttaaaatcttaaccatttatttattaataattataacttttattcagtaaaatgcagttctaacttaaagtcaagttaattttatataaattactttcctaatttggagaaatgattgatgcatatatcctttgaaatcacatatccccacttttgggttaggctgcataattgaagtggggcttgcaaaatatgagttacaggcaaccataattactgtgttgaaatttcagttggaaaagttcaaaactatagtttaaataaaatcattagatagatttttacgtaaatcataatattacaaactccttacgaattcccatggagcgtccctcggtcacatgtgcatcctctagtggagaagttgtaatgagttgctggcctctcttgggattgttcatcaaagtagctgtccctacacaccacaacagaatggtgttgtggagagaaagcacagacacatccttgagatggcaagggcacttaagtttcagagtggtgtacctaccaggttttggggagactgtgtcaagactgttgtatacctgatcaatagactccctactccaatcttacaaggcaaatgtccatatgaactactctatcagaaacatgccaagcttgatcacttgagagtgtttgggtgtcttgcttttgcagcaacactgcccaaaggtgacaaacttgcacctagagccaagcgaaccatcttcattggttattctgagactcaaaagggttacaggttgtatgacttggataataaggtgatcatagtgagcagggatgtagttttcagagagtttcagttccctttcaaagagtccatctcccatgaacctgatatgttcactcgagcaggttcacttagctctgaagatacagctttgcagctagtgtataatgatatttttccttccagctttcatactgatgatgatgatactccagttcttattcctgacattgtagaggccaatgaggatatcactttggggactcattctacccaaaatcatatcacttcagctgatgctgcaccttcaggccacctgcatgctcctgcagagtcagttgagtcagctgagcctgacctagcaaatgctgaatttgagcaccatactgctgtagctgatccctctctagttccagcaaacctgcacactagacctaaacgcaatgcaggtcctcccatctggctcaaggactttgtgacactgaacaaaggctctagggatgttccataccctatagccaactatgtttcctatgatcacttgcctgttcactatcaagcttatttgagtgctttctccacttatactgaacctaaatccttcaaagaagcagctcaagatgagaaatggatggaggccatgtcccttgagatacaggctcttgaggataataacacctgggagattgtccctttaccccctggtaaacagcctatagggtccaaatgggtgtacaaaattaaatacaaagctaatggtgaagttgacaggtttaaggcaaggctagtggccaagggatacactcagcaagaaggccttgactaccatgaaactttttctccagtggccaaaatggtcactgtaaaagctgtcatatctgttgctgcttccaagggctggttccttttccagatggatgtcaacaatgcttttctacaaggtgacctcatggaagaagtttatatgtctctgcctcagggttttcacagccaaggggaaaccaaagtgtgcaggctgttgaatccctctatggtttaaagcaagcatcaaggcagtggaacatcaagcttaccactgtccttatgcaggctggttttatgcaaagtgcttatgatcactccttgtttaccaaaagagaggggactgaccttgtcataatcctgatttatgtggatgatctgttaataacaggcagcagcaatgttttgatttcagaagcaaaggcaaccctgcatcagcatttcaagatgaaagatttgggagaactaaaatacttcttaggcattgaggtgctgagatcagaaaagggaatcctactgaaccagaggaagtatgcactggaactgatatcaggtgttggtctgggaggctgcagaccagtgtcaaccccaatggaacagaaccaaaggctgactactgtagaatatgacaagcacttaggaaagactgatgatgcagagttagaggatgtgggatcctatcagagactggtgggaaagcttctctacttgacaatcacaaggccagatatcagctttgcagtgcaggtgctgagtcaattcatgcagcaacccaaacagtcacacctggaagcagcattgagggttgtgaagtacataaaaggttctccaggtgttaggaacgctaagcttgaattcttgcggaggtttccatagcctaaggccacgagtgcacgtgttcataggctagttgtgttaaccttggggagcagtcgacaatttggattgcatcgaggtcttgccaaattttactttcaagacagtgatttcgacggcacaataaacttccaattagttatcttctcttttgttctgtattgtgtttttttctatttttctaacaatggaaatagtatataccggccaacagtgttgcactgttgtctggaccttgtcaaaggttcgacagtgcatagtttggactcgatgtggcatatgggggttctgataagtgatgagattatgaaaataatttaattttagctattaaaaatacaatacaactttcaaaagcgggaaacttgtttgattatgatctaggattctaggtctttttttttaagttcgagacagaaaaccaaaaacaaaacagctaataagaagaagaaaaacaatacagcaatattcccacaaaaataaatttgttcttttgcgtactagccataggatttaattatatatatgggccagtccagaagcattgtcactgatagatcatagactagtaaaaaagaatgaaccacttcagaacactaactatatatataaagatagattcatatctatttagatcgtgattattaatacaagaaaatatttgtatttcttaattttttgtttttaaatttatgtaaatatattgacatcttttttaacgaaaattcaaattttaattcattatattacaagaaattaacacattttactagaccaactctcatgttacaacactatatatgttgaacattacaagaatgccactttatctcacccctttaattaggaaaagcaaaaatctatttgcatataattttgtaccaaaaaattatacaataaaaaagaaaaagagaaaagtaaaagataagacgactgatgtaataaaaagagagaaaaaataatactaaaaaataatataaataacataactcttaaaaaaaaaaggtaacactgttgagtattttttttattacttttctctaaaaaggtatcatggtttcactaatgtccaggttttttttttccaaataaactcctttccggtggtaggtatcgtttcacacttgaatacttttttgcaatatttttcgtaacactttatttctctccattacataattttacccttgcctatgtctttattctgccacatataccctaagagtgagcaaatgtgtgcacgcatataacctgattggactatatatcggacttgatacaaatgtgtgctcacatgaagagaaaaaggattgaacccatgatcatcatcatgtcacatgcagatgccatgcattcaacaccaaagttatgatcagaaaacctctcaaggtaggtgactcaccaaatgaacaagccaattttttgcttcccaaatttcaccataaatagggacactactccatccaaaccatacacgaccacatacagtgggattagtaagtaaacaaattaattaataaccaaaagttataatttcaattgaatttagtcaatggtgtccaagggcgaagaggtgattaaagagttcatgaggttcaaagtgagaatggagggcagtatgaacggccatgagttcgagatcgaaggtgaaggcgaaggtagaccatacgagggcacccagacggcgaaactgaaagtgacgaagggcggcccactgcccttcgcctgggacattctgagtccacaatttatgtacggttccaaggcatacgttaagcaccccgccgacatccctgattacaaaaagctgtcgttcccggaggggtttaaatgggaaagagtgatgaattttgaggacggagggctcgtcactgtgactcaagactcctcacttcaagacggcaccctaatttacaaggtgaagatgaggggaacaaatttcccccctgatggccctgttatgcagaaaaaaacaatgggatgggaggcatccaccgagagactttacccgcgcgacggagtgctcaaaggcgaaatccatcaggcactgaaactaaaggacggcggccactacctggttgagttcaaaactatatatatggccaagaaacccgtccagttgccgggatattactatgtggacacaaaactggatataacgtcccacaacgaagattacaccatagtggaacagtatgagcgcagtgagggtcgtcaccatctattcctcggacatggcacgggttccacgggctcgggttccagcggcacggcaagctcggaggacaacaacatggcggtgatcaaagagttcatgcggtttaaagtccgtatggagggcagcatgaatgggcacgaatttgaaattgagggagaaggcgagggccgaccatatgaggggacacaaaccgcgaaactaaaagtcaccaaaggtgggccgcttccttttgcctgggacattctctctcctcagttcatgtatggctccaaggcctacgttaaacatccggctgatatccccgattataagaaattgtctttcccggaggggtttaaatgggagcgggttatgaatttcgaggatggtggactggttactgttacacaagattcgagtctgcaggatggcacattgatatacaaagtcaaaatgaggggtacgaattttcccccggacggcccggtgatgcagaaaaaaactatgggctgggaggccagcacggagaggctgtacccgagggacggggttttgaaaggggagatacaccaggcgcttaaactgaaggacggggggcactatctggtggaatttaagacgatatatatggcgaaaaagcccgtgcagctacctggctactactacgtcgataccaaactcgatattacctctcataacgaggactacactatcgtggagcaatatgaaagatcagaaggacgtcaccacctttttctatacggaatggacgagctttacaaaagttcgccaaagaagaagcggaaggtctaagcttgtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttatcttcacatataaatattgtttgaatatcttcaatcttttatttgtatgagacaattatttaagtatcgggtgacaatgcagctattatgtattgtcgattgttcgctggatccgaattcggcactcccagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 59 - DNA, 10767 bp, synthetic construct
  source 1..10767: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagtttgtaatattatgatttacgtaaaaatctatctaatgattttatttaaactatagttttgaacttttccaactgaaatttcaacacagtaattatggttgcctgtaactcatattttgcaagccccacttcaattatgcagcctaacccaaaagtggggatatgtgatttcaaaggatatatgcatcaatcatttctccaaattaggaaagtaatttatataaaattaacttgactttaagttagaactgcattttactgaataaaagttataattattaataaataaatggttaagattttaacgttataatatattcatacagtacaaattcaagtttttattcttgaaataaaatttttattttaggtatttcatttttatgcagtcatgtgtcttttttttatttttgaaacatattttatggtcatttatatcaattcaaattagaattttcttcctctctaactaattattcaacaacaaaaaaaaactattttttcaagtttttattttactcagtgttttaatcaatattaagcataatgagtatttataaatcattctcaaaataatattttacatgttaaataatttttaagtatttttaagagtgtgtttaattgtgttttaatttcttagtctattttttaaaaactattttatgaaacatttttcataaactgatgtttagagagtaaaagtggaaaagtaagatgtgaaaatacaaggaagaacatgaataatagaaaattaaaagtaaaaaacccaaacatcgatcatagagatgttatagttttttggttttgaaaaaactaaaaaaagagatagtagttttaaaaacagttttcgaaactatttttatcaaacaacataatttttttagcttttaaataaaaacagctttaaaaaatgtccaggtttttttccaaataaattcaattccatattttaataagtaatggttaatccagtggtaggtatatattgtttaacacttgaaatttttttccattcaatttttttaatatttttcataacacttaatttatttattacatatatcatatgttatattattatttttctctattctcttcctttctctctagttatttgtaaaaaaaaaaacatataatttatttccattgcataaattttatccatgtctatccatgcctatgtttttattctgccacatataccctaagtgttagcaaatatgtgcacacattaacctaattaaactatactagaactatatatcggacttgatagaaagtggagataagccaaggtagacaagaaaaaggattgaacacatgatcatcctcatgtcacatgcagttgccatggatccaacaccaaagttatgatcagaaaacctctcaaggcaggtgactcacgaaatgaacaagccaatttgcttccaaaattcaccataaatagggacactactcaatgcaaaccatgcatacacaatcatatacagtgggattagtaaacaaattaattaataaccaaaagttatttaaattgaagtcaatggctcaagttattaatactttcgacggtgttgcagactaccttcaaacttatcacaagctgccagataactatatcacaaaatctgaagctcaggccctcgggtgggtagcttcaaagggtaacctcgccgacgttgcacctggaaagagtattgggggcgacatctttagtaaccggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagtgaaggtaaattgcctggaaaatctggacgtacatggcgcgaggccgatataaattacacaagcggattccgtaactctgacagaatactttattcttcagactggctgatatataaaacaacagatcattatcaaacatttactaaaatacgttaggcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 60 - DNA, 16068 bp, synthetic construct
  source 1..16068: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggaggtttccatagcctaaggccacgagtgcacgtgttcataggctagttgtgttaaccttggggagcagtcgacaatttggattgcatcgaggtcttgccaaattttactttcaagacagtgatttcgacggcacaataaacttccaattagttatcttctcttttgttctgtattgtgtttttttctatttttctaacaatggaaatagtatataccggccaacagtgttgcactgttgtctggaccttgtcaaaggttcgacagtgcatagtttggactcgatgtggcatatgggggttctgataagtgatgagattatgaaaataatttaattttagctattaaaaatacaatacaactttcaaaagcgggaaacttgtttgattatgatctaggattctaggtctttttttttaagttcgagacagaaaaccaaaaacaaaacagctaataagaagaagaaaaacaatacagcaatattcccacaaaaataaatttgttcttttgcgtactagccataggatttaattatatatatgggccagtccagaagcattgtcactgatagatcatagactagtaaaaaagaatgaaccacttcagaacactaactatatatataaagatagattcatatctatttagatcgtgattattaatacaagaaaatatttgtatttcttaattttttgtttttaaatttatgtaaatatattgacatcttttttaacgaaaattcaaattttaattcattatattacaagaaattaacacattttactagaccaactctcatgttacaacactatatatgttgaacattacaagaatgccactttatctcacccctttaattaggaaaagcaaaaatctatttgcatataattttgtaccaaaaaattatacaataaaaaagaaaaagagaaaagtaaaagataagacgactgatgtaataaaaagagagaaaaaataatactaaaaaataatataaataacataactcttaaaaaaaaaaggtaacactgttgagtattttttttattacttttctctaaaaaggtatcatggtttcactaatgtccaggttttttttttccaaataaactcctttccggtggtaggtatcgtttcacacttgaatacttttttgcaatatttttcgtaacactttatttctctccattacataattttacccttgcctatgtctttattctgccacatataccctaagagtgagcaaatgtgtgcacgcatataacctgattggactatatatcggacttgatacaaatgtgtgctcacatgaagagaaaaaggattgaacccatgatcatcatcatgtcacatgcagatgccatgcattcaacaccaaagttatgatcagaaaacctctcaaggtaggtgactcaccaaatgaacaagccaattttttgcttcccaaatttcaccataaatagggacactactccatccaaaccatacacgaccacatacagtgggattagtaagtaaacaaattaattaataaccaaaagttataatttcaattgaatttagtcaatgaagaaggccgtcataaatggagaacagatcaggagtatcagcgatctccatcaaactctgaagaaagaactcgctctgccagagtattacggtgaaaaccttgatgctctttgggactgcctcactggttgggtcgagtatccactcgtcctggaatggagacagttcgaacaatccaaacagctcaccgagaatggagctgaaagtgttttgcaagtctttagggaagcaaaagccgaaggctgtgacatcaccattatactctcttaggcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaattcccatggagcgtccctcggtcacatgtgcatcctctagtggagaagttgtaatgagttgctggcctctcttgggattgttcatcaaagtagctgtccctacacaccacaacagaatggtgttgtggagagaaagcacagacacatccttgagatggcaagggcacttaagtttcagagtggtgtacctaccaggttttggggagactgtgtcaagactgttgtatacctgatcaatagactccctactccaatcttacaaggcaaatgtccatatgaactactctatcagaaacatgccaagcttgatcacttgagagtgtttgggtgtcttgcttttgcagcaacactgcccaaaggtgacaaacttgcacctagagccaagcgaaccatcttcattggttattctgagactcaaaagggttacaggttgtatgacttggataataaggtgatcatagtgagcagggatgtagttttcagagagtttcagttccctttcaaagagtccatctcccatgaacctgatatgttcactcgagcaggttcacttagctctgaagatacagctttgcagctagtgtataatgatatttttccttccagctttcatactgatgatgatgatactccagttcttattcctgacattgtagaggccaatgaggatatcactttggggactcattctacccaaaatcatatcacttcagctgatgctgcaccttcaggccacctgcatgctcctgcagagtcagttgagtcagctgagcctgacctagcaaatgctgaatttgagcaccatactgctgtagctgatccctctctagttccagcaaacctgcacactagacctaaacgcaatgcaggtcctcccatctggctcaaggactttgtgacactgaacaaaggctctagggatgttccataccctatagccaactatgtttcctatgatcacttgcctgttcactatcaagcttatttgagtgctttctccacttatactgaacctaaatccttcaaagaagcagctcaagatgagaaatggatggaggccatgtcccttgagatacaggctcttgaggataataacacctgggagattgtccctttaccccctggtaaacagcctatagggtccaaatgggtgtacaaaattaaatacaaagctaatggtgaagttgacaggtttaaggcaaggctagtggccaagggatacactcagcaagaaggccttgactaccatgaaactttttctccagtggccaaaatggtcactgtaaaagctgtcatatctgttgctgcttccaagggctggttccttttccagatggatgtcaacaatgcttttctacaaggtgacctcatggaagaagtttatatgtctctgcctcagggttttcacagccaaggggaaaccaaagtgtgcaggctgttgaatccctctatggtttaaagcaagcatcaaggcagtggaacatcaagcttaccactgtccttatgcaggctggttttatgcaaagtgcttatgatcactccttgtttaccaaaagagaggggactgaccttgtcataatcctgatttatgtggatgatctgttaataacaggcagcagcaatgttttgatttcagaagcaaaggcaaccctgcatcagcatttcaagatgaaagatttgggagaactaaaatacttcttaggcattgaggtgctgagatcagaaaagggaatcctactgaaccagaggaagtatgcactggaactgatatcaggtgttggtctgggaggctgcagaccagtgtcaaccccaatggaacagaaccaaaggctgactactgtagaatatgacaagcacttaggaaagactgatgatgcagagttagaggatgtgggatcctatcagagactggtgggaaagcttctctacttgacaatcacaaggccagatatcagctttgcagtgcaggtgctgagtcaattcatgcagcaacccaaacagtcacacctggaagcagcattgagggttgtgaagtacataaaaggttctccaggtgttaggaacgctaagcttgaattcttgcggaggacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatggtcaagactaatcttttccttttcttcatcttctcgcttctcctctcactttccagcgcggacatggtgtccaagggcgaagaggtgattaaagagttcatgaggttcaaagtgagaatggagggcagtatgaacggccatgagttcgagatcgaaggtgaaggcgaaggtagaccatacgagggcacccagacggcgaaactgaaagtgacgaagggcggcccactgcccttcgcctgggacattctgagtccacaatttatgtacggttccaaggcatacgttaagcaccccgccgacatccctgattacaaaaagctgtcgttcccggaggggtttaaatgggaaagagtgatgaattttgaggacggagggctcgtcactgtgactcaagactcctcacttcaagacggcaccctaatttacaaggtgaagatgaggggaacaaatttcccccctgatggccctgttatgcagaaaaaaacaatgggatgggaggcatccaccgagagactttacccgcgcgacggagtgctcaaaggcgaaatccatcaggcactgaaactaaaggacggcggccactacctggttgagttcaaaactatatatatggccaagaaacccgtccagttgccgggatattactatgtggacacaaaactggatataacgtcccacaacgaagattacaccatagtggaacagtatgagcgcagtgagggtcgtcaccatctattcctcggacatggcacgggttccacgggctcgggttccagcggcacggcaagctcggaggacaacaacatggcggtgatcaaagagttcatgcggtttaaagtccgtatggagggcagcatgaatgggcacgaatttgaaattgagggagaaggcgagggccgaccatatgaggggacacaaaccgcgaaactaaaagtcaccaaaggtgggccgcttccttttgcctgggacattctctctcctcagttcatgtatggctccaaggcctacgttaaacatccggctgatatccccgattataagaaattgtctttcccggaggggtttaaatgggagcgggttatgaatttcgaggatggtggactggttactgttacacaagattcgagtctgcaggatggcacattgatatacaaagtcaaaatgaggggtacgaattttcccccggacggcccggtgatgcagaaaaaaactatgggctgggaggccagcacggagaggctgtacccgagggacggggttttgaaaggggagatacaccaggcgcttaaactgaaggacggggggcactatctggtggaatttaagacgatatatatggcgaaaaagcccgtgcagctacctggctactactacgtcgataccaaactcgatattacctctcataacgaggactacactatcgtggagcaatatgaaagatcagaaggacgtcaccacctttttctatacggaatggacgagctttacaaaaaggacgagttgtaagcttgtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttatcttcacatataaatattgtttgaatatcttcaatcttttatttgtatgagacaattatttaagtatcgggtgacaatgcagctattatgtattgtcgattgttcgctggatccgaattcggcactcccagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 61 - DNA, 10515 bp, synthetic construct
  source 1..10515: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagtttgtaatattatgatttacgtaaaaatctatctaatgattttatttaaactatagttttgaacttttccaactgaaatttcaacacagtaattatggttgcctgtaactcatattttgcaagccccacttcaattatgcagcctaacccaaaagtggggatatgtgatttcaaaggatatatgcatcaatcatttctccaaattaggaaagtaatttatataaaattaacttgactttaagttagaactgcattttactgaataaaagttataattattaataaataaatggttaagattttaacgttataatatattcatacagtacaaattcaagtttttattcttgaaataaaatttttattttaggtatttcatttttatgcagtcatgtgtcttttttttatttttgaaacatattttatggtcatttatatcaattcaaattagaattttcttcctctctaactaattattcaacaacaaaaaaaaactattttttcaagtttttattttactcagtgttttaatcaatattaagcataatgagtatttataaatcattctcaaaataatattttacatgttaaataatttttaagtatttttaagagtgtgtttaattgtgttttaatttcttagtctattttttaaaaactattttatgaaacatttttcataaactgatgtttagagagtaaaagtggaaaagtaagatgtgaaaatacaaggaagaacatgaataatagaaaattaaaagtaaaaaacccaaacatcgatcatagagatgttatagttttttggttttgaaaaaactaaaaaaagagatagtagttttaaaaacagttttcgaaactatttttatcaaacaacataatttttttagcttttaaataaaaacagctttaaaaaatgtccaggtttttttccaaataaattcaattccatattttaataagtaatggttaatccagtggtaggtatatattgtttaacacttgaaatttttttccattcaatttttttaatatttttcataacacttaatttatttattacatatatcatatgttatattattatttttctctattctcttcctttctctctagttatttgtaaaaaaaaaaacatataatttatttccattgcataaattttatccatgtctatccatgcctatgtttttattctgccacatataccctaagtgttagcaaatatgtgcacacattaacctaattaaactatactagaactatatatcggacttgatagaaagtggagataagccaaggtagacaagaaaaaggattgaacacatgatcatcctcatgtcacatgcagttgccatggatccaacaccaaagttatgatcagaaaacctctcaaggcaggtgactcacgaaatgaacaagccaatttgcttccaaaattcaccataaatagggacactactcaatgcaaaccatgcatacacaatcatatacagtgggattagtaaacaaattaattaataaccaaaagttatttaaattgaagtcaatgaagaaggccgtcataaatggagaacagatcaggagtatcagcgatctccatcaaactctgaagaaagaactcgctctgccagagtattacggtgaaaaccttgatgctctttgggactgcctcactggttgggtcgagtatccactcgtcctggaatggagacagttcgaacaatccaaacagctcaccgagaatggagctgaaagtgttttgcaagtctttagggaagcaaaagccgaaggctgtgacatcaccattatactctcttaggcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 62 - DNA, 8102 bp, synthetic construct
  source 1..8102: mol_type = other DNA
tgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgactctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagtcgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgctccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctga

SEQ ID NO: 63 - DNA, 7954 bp, synthetic construct
  source 1..7954: mol_type = other DNA
atatttgaatatacttaatgttgatgttaaatattatttcatccatcttaaaataagtgtcattctatttattttatatagacaaaaaaaagttaataaattaataaaaaaacagtaattttataaaactaaccttagaattaatatttcattaaaaaactaaaattacacttattagaaatattagaaaaaaaatatcactactaaaaatattattttttatgacgaacattctaaaatggttatatgaaatcattttagaatgtcatgcgatgacaattgtaaatacaagattgtttaacaaagacgattttacaaaaattgtcttaaaaattattgttttatgacggtttgatgaaaaccgtcttaaaatgattgtcattctaaaacgattttcgtaaaatcctcttaaaagttagtgcttttaatttttatgtctccctctcctctctcatttctctccctctcactctctactacttcttccgttccaaaataattgttgtattaggttattttacataggtgaagtaataaatagatgaaatataatgataattttagaaaattaatcttatatcatcatttaatacatttatatatagttcatcattaataaataaaaaaataattaatgttacatttaaaaactaaaatgataaaagtaattaatatacattaagaaactaaaatgacatatttttcttatacgacaattataacacgatgagggagtaatgaatattagttaaaattagtttttgatcacctaatttataattttgattcaatttagtcctttagttattttgggttcaatttaatcctctaatttttaaaattattttttttactttccattccatcacaagttccaaatgaatttgaatttaatcaacttatggtagtatatatagaactttgaaagattattatctattgtaaaaaaattcaatcttaagattttttttgcataacatgattcctaaactactcatcaggtgtcttaaagatcagttactagtgtaatggagctagctacttggctcacatttcaggtattttacgtaaaagtccttacatgcactgtcgccctaacactttgtttatgctaaccttgtcatcaaaatcaaaatgcagaaaagggaaaagaaagaaaaaattcaaactggctttcccatccccataaagacttgctaaatataaggagaagaagtgaagattatgatcagacatcacgtgggaacccttcttagttaaaattgtaataattcaagagagagggtcaagagtcactggaaactctcaggtttcaattttcaactgtacaaaataaaaattaaagagatgcaggggtcagttaaaggaaataaatcttgtagatataaatatctatatatagattatggttggataactttttatagaagaagaaaagaaaaacttgtcttataaatttaaatcatcttacacatgcatgcacttgaattttatagaaactcttataagctaaatatataaattaattttaatttatgaaaatatatagtttgcttcattttactatttatttacttttgttataagtgtaaacaaggccatattcgaagtaaaaggccaaaaactcaaaatccaagaagcaaaatggtttctttattttttttatgctttaaaaatttgaagcttcggaattttgcgtgcaaacttttatgtgcatgtccctctcattgaaattgaatcaaaccccacattaacaaattaaagtcactttcgaaaagaccctttgattatctccctctcctttttcccatacatgaagaaatggatggtttatggagcatgaaaggtgaacctcaagtctctctctctctcttctctctcatgcttaaattctccaacacgagagtaaattttagagtgagtggtaattgtggagggtctatttttctgtcataaatacctgtctgctaggccaacccacttccatattttgcatcctttgtacctcagttccaggttaacaccaaacaacacatatagtgcagattcataaggtatagactttgttaatttgcaccctctcttaccctaaatcttgtgtctattgtattttatctctttccccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctga

SEQ ID NO: 64 - DNA, 6875 bp, synthetic construct
  source 1..6875: mol_type = other DNA
tgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgactctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagtcgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgctccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagcgcccagccgctaccaaaaaagctggacaggctaaaaaaaagaagggtgacggcagcgggtccggctccgggtccggttccgtcgatctcgatttggatctggaacttcgcctggggttcgcactcgatctcgaccttgaactgcgtcttggctttgctctcgatcttgatctggagttgaggcttggattcgct

SEQ ID NO: 65 - DNA, 595 bp, synthetic construct
  source 1..595: mol_type = other DNA
caaagcaccagtggtctagtggtagaatagtaccctgccacggtacagacccgggttcgattcccggctggtgcaaaaaaatgtgaatgtggatggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaaaatttataacatttacatcgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcatgatgtctttagaccagacagttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 66 - DNA, 5827 bp, synthetic construct
  source 1..5827: mol_type = other DNA
ggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctga

SEQ ID NO: 67 - DNA, 2111 bp, synthetic construct
  source 1..2111: mol_type = other DNA
gacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcattttgtatctctctatatctgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcatttctttgtgtctcagctcagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcatgggaaaagattcagaactcgttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 68 - DNA, 4603 bp, synthetic construct
  source 1..4603: mol_type = other DNA
ggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagcgcccagccgctaccaaaaaagctggacaggctaaaaaaaagaagggtgacggcagcgggtccggctccgggtccggttccgtcgatctcgatttggatctggaacttcgcctggggttcgcactcgatctcgaccttgaactgcgtcttggctttgctctcgatcttgatctggagttgaggcttggattcgcttag

SEQ ID NO: 69 - DNA, 22640 bp, synthetic construct
  source 1..22640: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagatatttgaatatacttaatgttgatgttaaatattatttcatccatcttaaaataagtgtcattctatttattttatatagacaaaaaaaagttaataaattaataaaaaaacagtaattttataaaactaaccttagaattaatatttcattaaaaaactaaaattacacttattagaaatattagaaaaaaaatatcactactaaaaatattattttttatgacgaacattctaaaatggttatatgaaatcattttagaatgtcatgcgatgacaattgtaaatacaagattgtttaacaaagacgattttacaaaaattgtcttaaaaattattgttttatgacggtttgatgaaaaccgtcttaaaatgattgtcattctaaaacgattttcgtaaaatcctcttaaaagttagtgcttttaatttttatgtctccctctcctctctcatttctctccctctcactctctactacttcttccgttccaaaataattgttgtattaggttattttacataggtgaagtaataaatagatgaaatataatgataattttagaaaattaatcttatatcatcatttaatacatttatatatagttcatcattaataaataaaaaaataattaatgttacatttaaaaactaaaatgataaaagtaattaatatacattaagaaactaaaatgacatatttttcttatacgacaattataacacgatgagggagtaatgaatattagttaaaattagtttttgatcacctaatttataattttgattcaatttagtcctttagttattttgggttcaatttaatcctctaatttttaaaattattttttttactttccattccatcacaagttccaaatgaatttgaatttaatcaacttatggtagtatatatagaactttgaaagattattatctattgtaaaaaaattcaatcttaagattttttttgcataacatgattcctaaactactcatcaggtgtcttaaagatcagttactagtgtaatggagctagctacttggctcacatttcaggtattttacgtaaaagtccttacatgcactgtcgccctaacactttgtttatgctaaccttgtcatcaaaatcaaaatgcagaaaagggaaaagaaagaaaaaattcaaactggctttcccatccccataaagacttgctaaatataaggagaagaagtgaagattatgatcagacatcacgtgggaacccttcttagttaaaattgtaataattcaagagagagggtcaagagtcactggaaactctcaggtttcaattttcaactgtacaaaataaaaattaaagagatgcaggggtcagttaaaggaaataaatcttgtagatataaatatctatatatagattatggttggataactttttatagaagaagaaaagaaaaacttgtcttataaatttaaatcatcttacacatgcatgcacttgaattttatagaaactcttataagctaaatatataaattaattttaatttatgaaaatatatagtttgcttcattttactatttatttacttttgttataagtgtaaacaaggccatattcgaagtaaaaggccaaaaactcaaaatccaagaagcaaaatggtttctttattttttttatgctttaaaaatttgaagcttcggaattttgcgtgcaaacttttatgtgcatgtccctctcattgaaattgaatcaaaccccacattaacaaattaaagtcactttcgaaaagaccctttgattatctccctctcctttttcccatacatgaagaaatggatggtttatggagcatgaaaggtgaacctcaagtctctctctctctcttctctctcatgcttaaattctccaacacgagagtaaattttagagtgagtggtaattgtggagggtctatttttctgtcataaatacctgtctgctaggccaacccacttccatattttgcatcctttgtacctcagttccaggttaacaccaaacaacacatatagtgcagattcataaggtatagactttgttaatttgcaccctctcttaccctaaatcttgtgtctattgtattttatctctttccccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaattcccatggggagatatttgaatatacttaatgttgatgttaaatattatttcatccatcttaaaataagtgtcattctatttattttatatagacaaaaaaaagttaataaattaataaaaaaacagtaattttataaaactaaccttagaattaatatttcattaaaaaactaaaattacacttattagaaatattagaaaaaaaatatcactactaaaaatattattttttatgacgaacattctaaaatggttatatgaaatcattttagaatgtcatgcgatgacaattgtaaatacaagattgtttaacaaagacgattttacaaaaattgtcttaaaaattattgttttatgacggtttgatgaaaaccgtcttaaaatgattgtcattctaaaacgattttcgtaaaatcctcttaaaagttagtgcttttaatttttatgtctccctctcctctctcatttctctccctctcactctctactacttcttccgttccaaaataattgttgtattaggttattttacataggtgaagtaataaatagatgaaatataatgataattttagaaaattaatcttatatcatcatttaatacatttatatatagttcatcattaataaataaaaaaataattaatgttacatttaaaaactaaaatgataaaagtaattaatatacattaagaaactaaaatgacatatttttcttatacgacaattataacacgatgagggagtaatgaatattagttaaaattagtttttgatcacctaatttataattttgattcaatttagtcctttagttattttgggttcaatttaatcctctaatttttaaaattattttttttactttccattccatcacaagttccaaatgaatttgaatttaatcaacttatggtagtatatatagaactttgaaagattattatctattgtaaaaaaattcaatcttaagattttttttgcataacatgattcctaaactactcatcaggtgtcttaaagatcagttactagtgtaatggagctagctacttggctcacatttcaggtattttacgtaaaagtccttacatgcactgtcgccctaacactttgtttatgctaaccttgtcatcaaaatcaaaatgcagaaaagggaaaagaaagaaaaaattcaaactggctttcccatccccataaagacttgctaaatataaggagaagaagtgaagattatgatcagacatcacgtgggaacccttcttagttaaaattgtaataattcaagagagagggtcaagagtcactggaaactctcaggtttcaattttcaactgtacaaaataaaaattaaagagatgcaggggtcagttaaaggaaataaatcttgtagatataaatatctatatatagattatggttggataactttttatagaagaagaaaagaaaaacttgtcttataaatttaaatcatcttacacatgcatgcacttgaattttatagaaactcttataagctaaatatataaattaattttaatttatgaaaatatatagtttgcttcattttactatttatttacttttgttataagtgtaaacaaggccatattcgaagtaaaaggccaaaaactcaaaatccaagaagcaaaatggtttctttattttttttatgctttaaaaatttgaagcttcggaattttgcgtgcaaacttttatgtgcatgtccctctcattgaaattgaatcaaaccccacattaacaaattaaagtcactttcgaaaagaccctttgattatctccctctcctttttcccatacatgaagaaatggatggtttatggagcatgaaaggtgaacctcaagtctctctctctctcttctctctcatgcttaaattctccaacacgagagtaaattttagagtgagtggtaattgtggagggtctatttttctgtcataaatacctgtctgctaggccaacccacttccatattttgcatcctttgtacctcagttccaggttaacaccaaacaacacatatagtgcagattcataaggtatagactttgttaatttgcaccctctcttaccctaaatcttgtgtctattgtattttatctctttccaatgggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggtaagtcttactctctcttttttggtctgtatttttaattttttgaagtatactatttgtactgacgctaataatcttttttcaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtacttcatgcttcaacgtgtaacttaagagatactgtgtgaaattttatatttccatacatttgcttgacctttgctttttgtcaatttttttccccttacaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaaggttcggtgtccaagggcgaagaggtgattaaagagttcatgaggttcaaagtgagaatggagggcagtatgaacggccatgagttcgagatcgaaggtgaaggcgaaggtagaccatacgagggcacccagacggcgaaactgaaagtgacgaagggcggcccactgcccttcgcctgggacattctgagtccacaatttatgtacggttccaaggcatacgttaagcaccccgccgacatccctgattacaaaaagctgtcgttcccggaggggtttaaatgggaaagagtgatgaattttgaggacggagggctcgtcactgtgactcaagactcctcacttcaagacggcaccctaatttacaaggtgaagatgaggggaacaaatttcccccctgatggccctgttatgcagaaaaaaacaatgggatgggaggcatccaccgagagactttacccgcgcgacggagtgctcaaaggcgaaatccatcaggcactgaaactaaaggacggcggccactacctggttgagttcaaaactatatatatggccaagaaacccgtccagttgccgggatattactatgtggacacaaaactggatataacgtcccacaacgaagattacaccatagtggaacagtatgagcgcagtgagggtcgtcaccatctattcctcggacatggcacgggttccacgggctcgggttccagcggcacggcaagctcggaggacaacaacatggcggtgatcaaagagttcatgcggtttaaagtccgtatggagggcagcatgaatgggcacgaatttgaaattgagggagaaggcgagggccgaccatatgaggggacacaaaccgcgaaactaaaagtcaccaaaggtgggccgcttccttttgcctgggacattctctctcctcagttcatgtatggctccaaggcctacgttaaacatccggctgatatccccgattataagaaattgtctttcccggaggggtttaaatgggagcgggttatgaatttcgaggatggtggactggttactgttacacaagattcgagtctgcaggatggcacattgatatacaaagtcaaaatgaggggtacgaattttcccccggacggcccggtgatgcagaaaaaaactatgggctgggaggccagcacggagaggctgtacccgagggacggggttttgaaaggggagatacaccaggcgcttaaactgaaggacggggggcactatctggtggaatttaagacgatatatatggcgaaaaagcccgtgcagctacctggctactactacgtcgataccaaactcgatattacctctcataacgaggactacactatcgtggagcaatatgaaagatcagaaggacgtcaccacctttttctatacggaatggacgagctttacaaagcttgtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttatcttcacatataaatattgtttgaatatcttcaatcttttatttgtatgagacaattatttaagtatcgggtgacaatgcagctattatgtattgtcgattgttcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 70 - DNA, 2630 bp, synthetic construct
  source 1..2630: mol_type = other DNA
gacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcatggagaatggtgcgcgcagtgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaaagaggagtgacttggttttgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaactctctctacaagtggtaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacattagatgagtgatataaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaagtttccgtgaatcaaaaatgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacacatgttctattgtaagatgttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 71 - DNA, 2111 bp, synthetic construct
  source 1..2111: mol_type = other DNA
gacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcatatcgtatagtaattagctggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacatcatttacgtaaacgtttgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaagggttgaagagtttatatagttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 72 - DNA, 2111 bp, synthetic construct
  source 1..2111: mol_type = other DNA
gacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcatacgttgcatgtgttctttagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggagtgtgtgtaaatgtgaggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacctaattctaaccgatattggttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 73 - DNA, 3674 bp, synthetic construct
  source 1..3674: mol_type = other DNA
cacatgtcacactctggtgcacatggtgttggtagaaagaccaaatgtcacgcccataactatcaatcgttgtcagtattgctatattccttggggatttacgactcattgacaaccagagtggttttaagacaactgtttttatctagctatggaagattctcacgatgctcaagaatttgaagtgatcaatggggtttgatgcaagaggtaatgtcctgagttggagatttgagttattacgactcgtataggtcattgaggattgaggagatcacaccgatatgatgcccgaggaggaggaagtgcatcatgagcttgtacgagatttagagccttcatcttaggaggaggattgattaccttcctgtgtttataccttaggcgtaattcaatacttttagtcgggtagtgatctgctctgaccctatatccttttttatatttgatgtatttgaggggtgagatcttcattgaaactgttttctttatgtaacagatgtgtatcgataatttaattacttttacactttgggttgtatccaaatttcttctattgtgacactatttatttgatttatttacatataaaggtttcatattataaatttgcgttcatttatttacatatttacttacgacgtgaaaacttgatgtttcttaagcaatgcatttattttattttatcttacttatacaaaaaaaatgatgagatatttttttaacttaaaaatagaaattaaaagttaatgacacgtattccacaatatttattattcaaaccattttatgtaatatttaaaaaaaaagtatatgagatgttacataaagtgaccataagttggaatacctcccactaacaaaaagtaacaaataaattactaggattggccttaaaaaaatcgcgagttgttaattaatttactccttaatattataatagtcaattaattaaattggtccctaaaattaattgttttcttaaaaaaaataagtaaaacatgagaatccaaatcaattttagttggtggttggtttgagtatttttttttcatttttaaagaaaaataaaataaaagtgaaaatacattttcttaaaatttagaaaatatacataaaaacagatcaatagtaaagcaacgttttcaatcttttcttaagaagtaaaaatacggtgacactttgacattttttctcaatatattttttaaatcttaaaaatttaaaataaaaaatatttttaaaaaataaaaataaaaataaaaaataaacactcaaactaaaataagtaaaagttttaattttcacaatgtggcgcattaagactctcgaaatgctatttccaaaaagaatggtagctgactagatgcaatctctcgtaggcgttaggttggcaatggaaggtttggttgcacgcttgcacgccccatgtcagctgataacctactttcttagagtaacccactttccatattcacaaatgattaatggtcaatcttctaattcacgcactactcaccccaaccatccattaggatattccgagtctaatggctccattattaaaaaaaaaaaatctttttatttaaattttaacttttcatttttatatatttattcttaaacgaaaaatctaaactactatatttgattcatattctttatttattttttaaaatttccccattgtttaaattttttaaatcaaaatcaaaatcatttaagtttacttaattttaatattgtatgaaataattaaaattaaaaaaaaatgtttttttaacaaagtaaaaagaaattataatggtgatagaaccacgggaaagtatttgtgcaaaataattgaaattacaagtattattttctattacaatttttttaattgtttctttgcttttttttttttttaacttctattttgcaagcatgtattttatatttaatgtgtgtggagggggacggagtacaaactcttttttgtctatattcagtaaaatgttggtggtgacgtgccttgcgtgatgaactaccatttcaaacaccacacgacaccacacatccattttaaatataaccccatcatagatatcccgaatcatcaaattattacttcatagcaactagcaaattaattagcttcaccaatggactcattgttacttctaaaagaaattgccacttccattttgatcttcttgatcactcgcctctccattcaaacattcctcaaaagctatcgccagaaactcccaccggggccaaaagggtggccagttgtgggtgcactccctctcatgggaagcatgcctcatgtcaccttagcaaagatggcaaaaaaatatggacctataatgtacctcaaaatgggcactaacaacatggttgtggcctctactccagctgctgctcgtgccttcctcaaaacccttgatcaaaacttttcaaaccggccctccaatgctggtgcaacccatttggcttatgatgcacgggatatggtgtttgctcattacggatcacggtggaagttgctaagaaaactaagtaacttgcacatgcttggaggaaaggcacttgatgattgggcccaaattcgagatgaagagatggggcacatgcttggtgcaatgtacgattgtaacaagagggatgaggctgtggtggtggcggagatgttgacatattcaatggccaacatgattggccaagttatattgagtcgtcgagtgtttgagacaaagggttcggagtctaacgagttcaaggacatggtggttgagctcatgaccgttgctggttacttcaacattggtgacttcataccctttttggccaagttggacttgcaaggcatagagcgtggcatgaagaagttgcacaagaagtttgatgcgttgttaacgagcatgattgaggagcatgttgcttctagtcacaagagaaagggcaagcccgatttcttagacatggtaatggctcatcatagtgagaactccgatggggaggaactatcgctcaccaacatcaaggcactactcttgaacctattcaccgcaggcaccgatacatcttcaagtataatagagtggtccttagccgagatgttgaagaagcccagcataatgaagaaggctcatgaagaaatggaccaagtcataggaagggatcgccgcctcaaagaatctgacataccaaagcttccctacttccaagccatttgcaaagaaacctatagaaagcacccttcaacacccctaaacctgcctcgaatctcatctgaaccgtgccaagtgaatggttactacattcccgagaacactaggctgaatgtgaacatttgggccataggaagagatcctgatgtgtggaacaatcctttggagtttatgcccgagaggtttttgagtgggaagaatgccaaaattgacccacgtgggaatgattttgagcttattccatttggtgctgggaggaggatttgtgcagggactaggatggggattgtgttggttcactacattttgggcactttggtgcattcgtttgattggaagctacccaatggggtgagggagttagacatggaggagtcctttgggcttgccttgcaaaaaaaggttccacttgctgctttggttacccctaggttgaacccaagtgcttacatttcttag

SEQ ID NO: 74 - DNA, 1114 bp, synthetic construct
  source 1..1114: mol_type = other DNA
caaagcaccagtggtctagtggtagaatagtaccctgccacggtacagacccgggttcgattcccggctggtgcatggagaatggtgcgcgcagtgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaaagaggagtgacttggttttgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaactctctctacaagtggtaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacattagatgagtgatataaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaagtttccgtgaatcaaaaatgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacacatgttctattgtaagatgttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 75 - DNA, 595 bp, synthetic construct; identical to SEQ ID NO: 65
  source 1..595: mol_type = other DNA

SEQ ID NO: 76 - DNA, 595 bp, synthetic construct
  source 1..595: mol_type = other DNA
caaagcaccagtggtctagtggtagaatagtaccctgccacggtacagacccgggttcgattcccggctggtgcattttgtatctctctatatctgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcatttctttgtgtctcagctcagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcatgggaaaagattcagaactcgttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 77 - DNA, 595 bp, synthetic construct
  source 1..595: mol_type = other DNA
caaagcaccagtggtctagtggtagaatagtaccctgccacggtacagacccgggttcgattcccggctggtgcatatcgtatagtaattagctggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacatcatttacgtaaacgtttgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaagggttgaagagtttatatagttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 78 - DNA, 595 bp, synthetic construct
  source 1..595: mol_type = other DNA
caaagcaccagtggtctagtggtagaatagtaccctgccacggtacagacccgggttcgattcccggctggtgcatacgttgcatgtgttctttagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggagtgtgtgtaaatgtgaggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacctaattctaaccgatattggttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgca

SEQ ID NO: 79 - DNA, 20 bp, synthetic construct
  source 1..20: mol_type = other DNA
ctccatcaacagcagatcca

SEQ ID NO: 80 - DNA, 20 bp, synthetic construct
  source 1..20: mol_type = other DNA
cgcacaatcccactatcctt

SEQ ID NO: 81 - DNA, 20 bp, synthetic construct
  source 1..20: mol_type = other DNA
gaaagaactcgctctgccag

SEQ ID NO: 82 - DNA, 21 bp, synthetic construct
  source 1..21: mol_type = other DNA
tgcaaaacactttcagctcca

SEQ ID NO: 83 - DNA, 20 bp, synthetic construct
  source 1..20: mol_type = other DNA
ttcgacggtgttgcagacta

SEQ ID NO: 84 - DNA, 21 bp, synthetic construct
  source 1..21: mol_type = other DNA
cggttactaaagatgtcgccc

SEQ ID NO: 85 - DNA, 22 bp, synthetic construct
  source 1..22: mol_type = other DNA
ccagttatcggtcatcttcttc

SEQ ID NO: 86 - DNA, 21 bp, synthetic construct
  source 1..21: mol_type = other DNA
taccacgagtccaagttctac

SEQ ID NO: 87 - DNA, 21 bp, synthetic construct
  source 1..21: mol_type = other DNA
tggctactactacgtcgatac

SEQ ID NO: 88 - DNA, 21 bp, synthetic construct
  source 1..21: mol_type = other DNA
gtgacgtccttctgatctttc

SEQ ID NO: 89 - DNA, 20 bp, synthetic construct
  source 1..20: mol_type = other DNA
gggtagcttcaaagggtaac

SEQ ID NO: 90 - DNA, 20 bp, synthetic construct
  source 1..20: mol_type = other DNA
tccaggcaatttaccttcac

SEQ ID NO: 91 - DNA, 22 bp, synthetic construct
  source 1..22: mol_type = other DNA
tggagaacagatcaggagtatc

SEQ ID NO: 92 - DNA, 20 bp, synthetic construct
  source 1..20: mol_type = other DNA
cagtcccaaagagcatcaag

SEQ ID NO: 93 - DNA, 22 bp, synthetic construct
  source 1..22: mol_type = other DNA
ccagttatcggtcatcttcttc

SEQ ID NO: 94 - DNA, 21 bp, synthetic construct
  source 1..21: mol_type = other DNA
taccacgagtccaagttctac

SEQ ID NO: 95 - DNA, 21 bp, synthetic construct
  source 1..21: mol_type = other DNA
tggctactactacgtcgatac

SEQ ID NO: 96 - DNA, 21 bp, synthetic construct
  source 1..21: mol_type = other DNA
gtgacgtccttctgatctttc

SEQ ID NO: 97 - DNA, 21 bp, synthetic construct
  source 1..21: mol_type = other DNA
ctggatctggcactcttattc

SEQ ID NO: 98 - DNA, 20 bp, synthetic construct
  source 1..20: mol_type = other DNA
gagctgagtttgcatgtttg

SEQ ID NO: 99 - DNA, 22936 bp, synthetic construct
  source 1..22936: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagtgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgactctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagtcgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgctccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaattcccatggggagtgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgactctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagtcgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgctaatgggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggtaagtcttactctctcttttttggtctgtatttttaattttttgaagtatactatttgtactgacgctaataatcttttttcaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtacttcatgcttcaacgtgtaacttaagagatactgtgtgaaattttatatttccatacatttgcttgacctttgctttttgtcaatttttttccccttacaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaaggttcggtgtccaagggcgaagaggtgattaaagagttcatgaggttcaaagtgagaatggagggcagtatgaacggccatgagttcgagatcgaaggtgaaggcgaaggtagaccatacgagggcacccagacggcgaaactgaaagtgacgaagggcggcccactgcccttcgcctgggacattctgagtccacaatttatgtacggttccaaggcatacgttaagcaccccgccgacatccctgattacaaaaagctgtcgttcccggaggggtttaaatgggaaagagtgatgaattttgaggacggagggctcgtcactgtgactcaagactcctcacttcaagacggcaccctaatttacaaggtgaagatgaggggaacaaatttcccccctgatggccctgttatgcagaaaaaaacaatgggatgggaggcatccaccgagagactttacccgcgcgacggagtgctcaaaggcgaaatccatcaggcactgaaactaaaggacggcggccactacctggttgagttcaaaactatatatatggccaagaaacccgtccagttgccgggatattactatgtggacacaaaactggatataacgtcccacaacgaagattacaccatagtggaacagtatgagcgcagtgagggtcgtcaccatctattcctcggacatggcacgggttccacgggctcgggttccagcggcacggcaagctcggaggacaacaacatggcggtgatcaaagagttcatgcggtttaaagtccgtatggagggcagcatgaatgggcacgaatttgaaattgagggagaaggcgagggccgaccatatgaggggacacaaaccgcgaaactaaaagtcaccaaaggtgggccgcttccttttgcctgggacattctctctcctcagttcatgtatggctccaaggcctacgttaaacatccggctgatatccccgattataagaaattgtctttcccggaggggtttaaatgggagcgggttatgaatttcgaggatggtggactggttactgttacacaagattcgagtctgcaggatggcacattgatatacaaagtcaaaatgaggggtacgaattttcccccggacggcccggtgatgcagaaaaaaactatgggctgggaggccagcacggagaggctgtacccgagggacggggttttgaaaggggagatacaccaggcgcttaaactgaaggacggggggcactatctggtggaatttaagacgatatatatggcgaaaaagcccgtgcagctacctggctactactacgtcgataccaaactcgatattacctctcataacgaggactacactatcgtggagcaatatgaaagatcagaaggacgtcaccacctttttctatacggaatggacgagctttacaaagcttgtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttatcttcacatataaatattgtttgaatatcttcaatcttttatttgtatgagacaattatttaagtatcgggtgacaatgcagctattatgtattgtcgattgttcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 100 - DNA, 10767 bp, synthetic construct; identical to SEQ ID NO: 59
  source 1..10767: mol_type = other DNA

SEQ ID NO: 101 - DNA, 16068 bp, synthetic construct; identical to SEQ ID NO: 60
  source 1..16068: mol_type = other DNA

SEQ ID NO: 102 - DNA, 10767 bp, synthetic construct; identical to SEQ ID NO: 59
  source 1..10767: mol_type = other DNA

SEQ ID NO: 103 - DNA, 12618 bp, synthetic construct
  source 1..12618: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagcacatgtcacactctggtgcacatggtgttggtagaaagaccaaatgtcacgcccataactatcaatcgttgtcagtattgctatattccttggggatttacgactcattgacaaccagagtggttttaagacaactgtttttatctagctatggaagattctcacgatgctcaagaatttgaagtgatcaatggggtttgatgcaagaggtaatgtcctgagttggagatttgagttattacgactcgtataggtcattgaggattgaggagatcacaccgatatgatgcccgaggaggaggaagtgcatcatgagcttgtacgagatttagagccttcatcttaggaggaggattgattaccttcctgtgtttataccttaggcgtaattcaatacttttagtcgggtagtgatctgctctgaccctatatccttttttatatttgatgtatttgaggggtgagatcttcattgaaactgttttctttatgtaacagatgtgtatcgataatttaattacttttacactttgggttgtatccaaatttcttctattgtgacactatttatttgatttatttacatataaaggtttcatattataaatttgcgttcatttatttacatatttacttacgacgtgaaaacttgatgtttcttaagcaatgcatttattttattttatcttacttatacaaaaaaaatgatgagatatttttttaacttaaaaatagaaattaaaagttaatgacacgtattccacaatatttattattcaaaccattttatgtaatatttaaaaaaaaagtatatgagatgttacataaagtgaccataagttggaatacctcccactaacaaaaagtaacaaataaattactaggattggccttaaaaaaatcgcgagttgttaattaatttactccttaatattataatagtcaattaattaaattggtccctaaaattaattgttttcttaaaaaaaataagtaaaacatgagaatccaaatcaattttagttggtggttggtttgagtatttttttttcatttttaaagaaaaataaaataaaagtgaaaatacattttcttaaaatttagaaaatatacataaaaacagatcaatagtaaagcaacgttttcaatcttttcttaagaagtaaaaatacggtgacactttgacattttttctcaatatattttttaaatcttaaaaatttaaaataaaaaatatttttaaaaaataaaaataaaaataaaaaataaacactcaaactaaaataagtaaaagttttaattttcacaatgtggcgcattaagactctcgaaatgctatttccaaaaagaatggtagctgactagatgcaatctctcgtaggcgttaggttggcaatggaaggtttggttgcacgcttgcacgccccatgtcagctgataacctactttcttagagtaacccactttccatattcacaaatgattaatggtcaatcttctaattcacgcactactcaccccaaccatccattaggatattccgagtctaatggctccattattaaaaaaaaaaaatctttttatttaaattttaacttttcatttttatatatttattcttaaacgaaaaatctaaactactatatttgattcatattctttatttattttttaaaatttccccattgtttaaattttttaaatcaaaatcaaaatcatttaagtttacttaattttaatattgtatgaaataattaaaattaaaaaaaaatgtttttttaacaaagtaaaaagaaattataatggtgatagaaccacgggaaagtatttgtgcaaaataattgaaattacaagtattattttctattacaatttttttaattgtttctttgcttttttttttttttaacttctattttgcaagcatgtattttatatttaatgtgtgtggagggggacggagtacaaactcttttttgtctatattcagtaaaatgttggtggtgacgtgccttgcgtgatgaactaccatttcaaacaccacacgacaccacacatccattttaaatataaccccatcatagatatcccgaatcatcaaattattacttcatagcaactagcaaattaattagcttcaccaatggactcattgttacttctaaaagaaattgccacttccattttgatcttcttgatcactcgcctctccattcaaacattcctcaaaagctatcgccagaaactcccaccggggccaaaagggtggccagttgtgggtgcactccctctcatgggaagcatgcctcatgtcaccttagcaaagatggcaaaaaaatatggacctataatgtacctcaaaatgggcactaacaacatggttgtggcctctactccagctgctgctcgtgccttcctcaaaacccttgatcaaaacttttcaaaccggccctccaatgctggtgcaacccatttggcttatgatgcacgggatatggtgtttgctcattacggatcacggtggaagttgctaagaaaactaagtaacttgcacatgcttggaggaaaggcacttgatgattgggcccaaattcgagatgaagagatggggcacatgcttggtgcaatgtacgattgtaacaagagggatgaggctgtggtggtggcggagatgttgacatattcaatggccaacatgattggccaagttatattgagtcgtcgagtgtttgagacaaagggttcggagtctaacgagttcaaggacatggtggttgagctcatgaccgttgctggttacttcaacattggtgacttcataccctttttggccaagttggacttgcaaggcatagagcgtggcatgaagaagttgcacaagaagtttgatgcgttgttaacgagcatgattgaggagcatgttgcttctagtcacaagagaaagggcaagcccgatttcttagacatggtaatggctcatcatagtgagaactccgatggggaggaactatcgctcaccaacatcaaggcactactcttgaacctattcaccgcaggcaccgatacatcttcaagtataatagagtggtccttagccgagatgttgaagaagcccagcataatgaagaaggctcatgaagaaatggaccaagtcataggaagggatcgccgcctcaaagaatctgacataccaaagcttccctacttccaagccatttgcaaagaaacctatagaaagcacccttcaacacccctaaacctgcctcgaatctcatctgaaccgtgccaagtgaatggttactacattcccgagaacactaggctgaatgtgaacatttgggccataggaagagatcctgatgtgtggaacaatcctttggagtttatgcccgagaggtttttgagtgggaagaatgccaaaattgacccacgtgggaatgattttgagcttattccatttggtgctgggaggaggatttgtgcagggactaggatggggattgtgttggttcactacattttgggcactttggtgcattcgtttgattggaagctacccaatggggtgagggagttagacatggaggagtcctttgggcttgccttgcaaaaaaaggttccacttgctgctttggttacccctaggttgaacccaagtgcttacatttcttaggcttaattggttgggttcgaatattcaccagctatgttctctagccttattttgttgtccaatgattttgtggctgtggctacataaataagtaatgtttgggttgcacaacctatttgtatttgtaaggttctatgttacttggaaatccgttaccccaccacctgcaaggcttagatttttattcttcatggatctctaatttagctgtcttgttttggtttaattatatttttaattctcactcaggtagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 104 - DNA, 18993 bp, synthetic construct
  source 1..18993: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagatatttgaatatacttaatgttgatgttaaatattatttcatccatcttaaaataagtgtcattctatttattttatatagacaaaaaaaagttaataaattaataaaaaaacagtaattttataaaactaaccttagaattaatatttcattaaaaaactaaaattacacttattagaaatattagaaaaaaaatatcactactaaaaatattattttttatgacgaacattctaaaatggttatatgaaatcattttagaatgtcatgcgatgacaattgtaaatacaagattgtttaacaaagacgattttacaaaaattgtcttaaaaattattgttttatgacggtttgatgaaaaccgtcttaaaatgattgtcattctaaaacgattttcgtaaaatcctcttaaaagttagtgcttttaatttttatgtctccctctcctctctcatttctctccctctcactctctactacttcttccgttccaaaataattgttgtattaggttattttacataggtgaagtaataaatagatgaaatataatgataattttagaaaattaatcttatatcatcatttaatacatttatatatagttcatcattaataaataaaaaaataattaatgttacatttaaaaactaaaatgataaaagtaattaatatacattaagaaactaaaatgacatatttttcttatacgacaattataacacgatgagggagtaatgaatattagttaaaattagtttttgatcacctaatttataattttgattcaatttagtcctttagttattttgggttcaatttaatcctctaatttttaaaattattttttttactttccattccatcacaagttccaaatgaatttgaatttaatcaacttatggtagtatatatagaactttgaaagattattatctattgtaaaaaaattcaatcttaagattttttttgcataacatgattcctaaactactcatcaggtgtcttaaagatcagttactagtgtaatggagctagctacttggctcacatttcaggtattttacgtaaaagtccttacatgcactgtcgccctaacactttgtttatgctaaccttgtcatcaaaatcaaaatgcagaaaagggaaaagaaagaaaaaattcaaactggctttcccatccccataaagacttgctaaatataaggagaagaagtgaagattatgatcagacatcacgtgggaacccttcttagttaaaattgtaataattcaagagagagggtcaagagtcactggaaactctcaggtttcaattttcaactgtacaaaataaaaattaaagagatgcaggggtcagttaaaggaaataaatcttgtagatataaatatctatatatagattatggttggataactttttatagaagaagaaaagaaaaacttgtcttataaatttaaatcatcttacacatgcatgcacttgaattttatagaaactcttataagctaaatatataaattaattttaatttatgaaaatatatagtttgcttcattttactatttatttacttttgttataagtgtaaacaaggccatattcgaagtaaaaggccaaaaactcaaaatccaagaagcaaaatggtttctttattttttttatgctttaaaaatttgaagcttcggaattttgcgtgcaaacttttatgtgcatgtccctctcattgaaattgaatcaaaccccacattaacaaattaaagtcactttcgaaaagaccctttgattatctccctctcctttttcccatacatgaagaaatggatggtttatggagcatgaaaggtgaacctcaagtctctctctctctcttctctctcatgcttaaattctccaacacgagagtaaattttagagtgagtggtaattgtggagggtctatttttctgtcataaatacctgtctgctaggccaacccacttccatattttgcatcctttgtacctcagttccaggttaacaccaaacaacacatatagtgcagattcataaggtatagactttgttaatttgcaccctctcttaccctaaatcttgtgtctattgtattttatctctttccccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaattcccatggggaggacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcatacgttgcatgtgttctttagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggagtgtgtgtaaatgtgaggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacctaattctaaccgatattggttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgcagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 105 - DNA, 18993 bp, synthetic construct
  source 1..18993: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagatatttgaatatacttaatgttgatgttaaatattatttcatccatcttaaaataagtgtcattctatttattttatatagacaaaaaaaagttaataaattaataaaaaaacagtaattttataaaactaaccttagaattaatatttcattaaaaaactaaaattacacttattagaaatattagaaaaaaaatatcactactaaaaatattattttttatgacgaacattctaaaatggttatatgaaatcattttagaatgtcatgcgatgacaattgtaaatacaagattgtttaacaaagacgattttacaaaaattgtcttaaaaattattgttttatgacggtttgatgaaaaccgtcttaaaatgattgtcattctaaaacgattttcgtaaaatcctcttaaaagttagtgcttttaatttttatgtctccctctcctctctcatttctctccctctcactctctactacttcttccgttccaaaataattgttgtattaggttattttacataggtgaagtaataaatagatgaaatataatgataattttagaaaattaatcttatatcatcatttaatacatttatatatagttcatcattaataaataaaaaaataattaatgttacatttaaaaactaaaatgataaaagtaattaatatacattaagaaactaaaatgacatatttttcttatacgacaattataacacgatgagggagtaatgaatattagttaaaattagtttttgatcacctaatttataattttgattcaatttagtcctttagttattttgggttcaatttaatcctctaatttttaaaattattttttttactttccattccatcacaagttccaaatgaatttgaatttaatcaacttatggtagtatatatagaactttgaaagattattatctattgtaaaaaaattcaatcttaagattttttttgcataacatgattcctaaactactcatcaggtgtcttaaagatcagttactagtgtaatggagctagctacttggctcacatttcaggtattttacgtaaaagtccttacatgcactgtcgccctaacactttgtttatgctaaccttgtcatcaaaatcaaaatgcagaaaagggaaaagaaagaaaaaattcaaactggctttcccatccccataaagacttgctaaatataaggagaagaagtgaagattatgatcagacatcacgtgggaacccttcttagttaaaattgtaataattcaagagagagggtcaagagtcactggaaactctcaggtttcaattttcaactgtacaaaataaaaattaaagagatgcaggggtcagttaaaggaaataaatcttgtagatataaatatctatatatagattatggttggataactttttatagaagaagaaaagaaaaacttgtcttataaatttaaatcatcttacacatgcatgcacttgaattttatagaaactcttataagctaaatatataaattaattttaatttatgaaaatatatagtttgcttcattttactatttatttacttttgttataagtgtaaacaaggccatattcgaagtaaaaggccaaaaactcaaaatccaagaagcaaaatggtttctttattttttttatgctttaaaaatttgaagcttcggaattttgcgtgcaaacttttatgtgcatgtccctctcattgaaattgaatcaaaccccacattaacaaattaaagtcactttcgaaaagaccctttgattatctccctctcctttttcccatacatgaagaaatggatggtttatggagcatgaaaggtgaacctcaagtctctctctctctcttctctctcatgcttaaattctccaacacgagagtaaattttagagtgagtggtaattgtggagggtctatttttctgtcataaatacctgtctgctaggccaacccacttccatattttgcatcctttgtacctcagttccaggttaacaccaaacaacacatatagtgcagattcataaggtatagactttgttaatttgcaccctctcttaccctaaatcttgtgtctattgtattttatctctttccccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaattcccatggggaggacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcatatcgtatagtaattagctggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacatcatttacgtaaacgtttgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaagggttgaagagtttatatagttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgcagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 106 - DNA, 19141 bp, synthetic construct
  source 1..19141: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagtgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgactctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagtcgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgctccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaattcccatggggaggacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcattttgtatctctctatatctgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcatttctttgtgtctcagctcagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcatgggaaaagattcagaactcgttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgcagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 107 - DNA, 19141 bp, synthetic construct
  source 1..19141: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagtgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgactctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagtcgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgctccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagaggcctgccgccacaaagaaagccggccaggcaaagaaaaaaaaacccaggggagggagtggtggactcctcgaccctggcactccaatggacgccgatctcgtagcatcatctaccgtcgtgtgggagcaggacgccgacccttttgctggcacagctgatgatttcccagcttttaatgaggaagaattggcttggctgatggagcttctcccacaaggtggtagcggcgggctcctcgatcccggtactcccatggacgcagatcttgttgctagctccactgttgtatgggagcaggacgccgatcccttcgctggaaccgccgatgatttcccagcattcaacgaggaagaacttgcatggctcatggagctcttgccccaagctcgcggtggatctgggggtttgttggatcccggaaccccaatggacgcagacctcgtggcttctagtaccgtagtatgggaacaggacgctgaccctttcgccggcaccgcagatgacttcccagcattcaacgaggaggaactggcctggctgatggaacttctgcctcagggtggatccggaggcctcttggaccccggtacccctatggacgctgacctggtcgcatcttctaccgtggtttgggagcaagatgccgacccattcgcaggaacagctgatgacttccccgcattcaatgaagaagagctcgcatggctgatggaattgcttccacaagccagaggcggctctggagggcttcttgatcccggaacacccatggatgccgacctcgtggcctctagcaccgtggtatgggaacaagacgcagatccattcgcagggaccgcagacgatttccccgcttttaacgaggaggagcttgcttggctcatggagctgctgcctcagggtggatccgggggacttctcgacccaggcacacccatggatgccgatctcgtcgccagttctaccgttgtctgggagcaagacgcagaccctttcgccggaactgcagacgacttccctgcctttaatgaagaagaactggcttggctcatggaactgctcccccaggctagagggggtagcggcgggggcggcagcggaggtgatgcacttgacgacttcgatcttgatatgcttggcagcgacgcacttgatgacttcgatttggatatgctggggtccgatgctcttgacgattttgatttggacatgttgggttcagacgcactcgatgatttcgatttggacatgctggcacgtggaagcgatgcccttgatgactttgacctggatatgctcgggtccgatgctttggacgacttcgacctcgacatgttgggaagtgatgctcttgatgatttcgatcttgatatgcttgggtccgacgctctcgatgattttgacctcgatatgctctgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaattcccatggggaggacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcaaaaaaatgtgaatgtggatggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaaaatttataacatttacatcgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcatgatgtctttagaccagacagttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgcagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 108 - DNA, 18436 bp, synthetic construct
  source 1..18436: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagtgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgactctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagtcgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgctccatggattacaaagatgacgatgataaagactacaaggacgacgacgataaaatggcccctaaaaagaagcgcaaggtgggtattcatggggtgcctgcagctggaatggataagaagtactccatcggactggctatcggaactaactccgtgggatgggctgtgatcactgatgagtacaaggtgccatccaagaagttcaaggtgctgggaaacactgatagacactccatcaagaagaacctgatcggagctctgctgttcgattccggagagactgctgaggctactagactgaagagaactgctagaagaagatacactagaagaaagaacagaatctgctacctgcaagagatcttctccaacgagatggctaaggtggatgattccttcttccacagactggaggagtccttcctggtggaggaggataagaagcacgagagacacccaatcttcggaaacatcgtggatgaggtggcttaccacgagaagtacccaactatctaccacctgagaaagaagctggtggattccactgataaggctgatctgagactgatctacctggctctggctcacatgatcaagttcagaggacacttcctgatcgagggagatctgaacccagataactccgatgtggataagctgttcatccaactggtgcaaacttacaaccaactgttcgaggagaacccaatcaacgcttccggagtggatgctaaggctatcctgtccgctagactgtccaagtccaggagactggagaacctgatcgctcaactgccaggagagaagaagaacggactgttcggaaacctgatcgctctgtccctgggactgactccaaacttcaagtccaacttcgatctggctgaggatgctaagctgcaactgtccaaggatacttacgatgatgatctggataacctgctggctcaaatcggagatcaatacgctgatctgttcctggctgctaagaacctgtccgatgctatcctgctgtccgatatcctgagagtgaacactgagatcactaaggctccactgtccgcttccatgatcaagagatacgatgagcaccaccaagatctgactctgctgaaggctctggtgagacaacaactgccagagaagtacaaggagatcttcttcgatcaatccaagaacggatacgctggatacatcgatggaggagcttcccaagaggagttctacaagttcatcaagccaatcctggagaagatggatggaactgaggagctgctggtgaagctgaacagagaggatctgctgagaaagcaaagaactttcgataacggatccatcccacaccaaatccacctgggagagctgcacgctatcctgaggagacaagaggatttctacccattcctgaaggataacagagagaagatcgagaagatcctgactttcagaatcccatactacgtgggaccactggctagaggaaactccagattcgcttggatgactagaaagtccgaggagactatcactccatggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggtggataagggagcttccgctcaatccttcatcgagagaatgactaacttcgataagaacctgccaaacgagaaggtgctgccaaagcactccctgctgtacgagtacttcactgtgtacaacgagctgactaaggtgaagtacgtgactgagggaatgagaaagccagctttcctgtccggagagcaaaagaaggctatcgtggatctgctgttcaagactaacagaaaggtgactgtgaagcaactgaaggaggattacttcaagaagatcgagtgcttcgattccgtggagatctccggagtggaggatagattcaacgcttccctgggaacttaccacgatctgctgaagatcatcaaggataaggatttcctggataacgaggagaacgaggatatcctggaggatatcgtgctgactctgactctgttcgaggatagagagatgatcgaggagagacttaagacttacgctcacctgttcgatgataaggtgatgaagcaactgaagagaagaagatacactggatggggtagactgtccagaaagctgatcaacggaatcagagataagcaatccggaaagactatcctggatttcctgaagtccgatggattcgctaacagaaacttcatgcaactgatccacgatgattccctgactttcaaggaggatatccaaaaggctcaagtgtccggacaaggagattccctgcacgagcacatcgctaacctggctggatccccagctatcaagaagggaatcctgcaaactgtgaaggtggtggatgagctggtgaaggtgatgggtagacacaagccagagaacatcgtgatcgagatggctagagagaaccaaactactcaaaagggacaaaagaactccagagagagaatgaagagaatcgaggagggaatcaaggagctgggatcccaaatcctgaaggagcacccagtggagaacactcaactgcaaaacgagaagctgtacctgtactacctgcaaaacggaagagatatgtacgtggatcaagagctggatatcaacagactgtccgattacgatgtggatgcaatcgtgccacaatccttcctgaaggatgattccatcgataacaaggtgctgactagatccgataagaacagaggaaagtccgataacgtgccatccgaggaggtggtgaagaagatgaagaactactggagacaactgctgaacgctaagctgatcactcaaagaaagttcgataacctgactaaggctgagagaggaggactgtccgagctggataaggctggattcatcaagagacaactggtggagactagacaaatcactaagcacgtggctcaaatcctggattccagaatgaacactaagtacgatgagaacgataagctgatcagagaggtgaaggtgatcactctgaagtccaagctggtgtccgatttcagaaaggatttccaattctacaaggtgagagagatcaacaactaccaccacgctcacgatgcttacctgaacgctgtggtgggaactgctctgatcaagaagtacccaaagctggagtccgagttcgtgtacggagattacaaggtgtacgatgtgagaaagatgatcgctaagtccgagcaagagatcggaaaggctactgctaagtacttcttctactccaacatcatgaacttcttcaagactgagatcactctggctaacggagagatcagaaaaagaccactgatcgagactaacggagagactggagagatcgtgtgggataagggaagagatttcgctactgtgagaaaggtgctgtccatgccacaagtgaacatcgtgaaaaagactgaggtgcaaactggaggattctccaaggagtccatcctgccaaagagaaactccgataagctgatcgctagaaagaaggattgggatccaaagaagtacggaggattcgattccccaactgtggcttactccgtgctggtggtggctaaggtggagaagggaaagtccaagaagctgaagtccgtgaaggagctgctgggaatcactatcatggagagatcctccttcgagaagaacccaatcgatttcctggaggctaagggatacaaggaggtgaagaaggatctgatcatcaagctgccaaagtactccctgttcgagctggagaacggaagaaagagaatgctggcttccgctggagagctgcaaaagggaaacgagctggctctgccatccaagtacgtgaacttcctgtacctggcttcccactacgagaagctgaagggatccccagaggataacgagcaaaagcaactgttcgtggagcaacacaagcactacctggatgagatcatcgagcaaatctccgagttctccaagagagtgatcctggctgatgctaacctggataaggtgctgtccgcttacaacaagcacagagataagccaatcagagagcaagctgagaacatcatccacctgttcactctgactaacctgggagctccagctgctttcaagtacttcgatactactatcgatagaaagagatacacttccactaaggaggtgctggatgctactctgatccaccaatccatcactggactgtacgagactagaatcgatctgtcccaactgggaggagatagttcgaagcgcccagccgctaccaaaaaagctggacaggctaaaaaaaagaagggtgacggcagcgggtccggctccgggtccggttccgtcgatctcgatttggatctggaacttcgcctggggttcgcactcgatctcgaccttgaactgcgtcttggctttgctctcgatcttgatctggagttgaggcttggattcgcttaggcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaattcccatggggaggacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcatggagaatggtgcgcgcagtgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaaagaggagtgacttggttttgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaactctctctacaagtggtaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacattagatgagtgatataaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaagtttccgtgaatcaaaaatgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacacatgttctattgtaagatgttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgcagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 109 - DNA, 16501 bp, synthetic construct; identical to SEQ ID NO: 58
  source 1..16501: mol_type = other DNA